SEQUENCE LISTING (1 sequence)

SEQ ID NO: 1 - protein, 12 aa, Artificial Sequence
  Synthetic Construct
VLLPKKSFAEPG

(19) United States
(12) Patent Application Publication
(10) Pub. No.: US 2017/0275270 A1
(43) Pub. Date: Sep. 28, 2017

(54) BENZYL SUBSTITUTED INDAZOLES

(71) Applicant: Bayer Pharma Aktiengesellschaft, Berlin (DE)

(72) Inventors: Lars BÄRFACKER, Düsseldorf (DE); Thomas MÜLLER, Frankfurt (DE); Anne MENGEL, Berlin (DE); Marion HITCHCOCK, Brookline, MA (US); Arwed CLEVE, Berlin (DE); Hans BRIEM, Berlin (DE); Gerhard SIEMEISTER, Berlin (DE); Wilhelm BONE, Berlin (DE); Amaury Ernesto FERNANDEZ-MONTALVAN, Berlin (DE); Jens SCHRÖDER, Berlin (DE); Ursula MÖNNING, Woltersdorf (DE)

(73) Assignee: Bayer Pharma Aktiengesellschaft, Berlin (DE)

(21) Appl. No.: 15/512,507

(22) PCT Filed: Sep. 15, 2015
(86) PCT No.: PCT/EP2015/071021

§ 371 Date: Mar. 17, 2017

(30) Foreign Application Priority Data
Sep. 19, 2014 (EP) 14185599.9
Sep. 22, 2014 (EP) 14185839.9

(51) Int. Cl. C07D 401/14; A61K 31/5377; C07D 405/14; C07F 7/18; A61K 31/695; A61K 31/517; C07D 403/14; C07D 487/04; A61K 31/519; A61K 31/53; C07D 417/14; C07D 491/052; C07D 491/048; C07D 471/04; C07D 495/04; A61K 45/06; A61K 31/506

(52) U.S. Cl. CPC C07D 401/14; A61K 31/506; A61K 31/5377; C07D 405/14; C07F 7/1804; A61K 31/695; A61K 31/517; C07D 403/14; C07D 487/04; A61K 31/519; A61K 31/53; C07D 417/14; C07D 491/052; C07D 491/048; C07D 471/04; C07D 495/04; A61K 45/06

(57) ABSTRACT

Compounds of formula (I) and their use as pharmaceuticals.

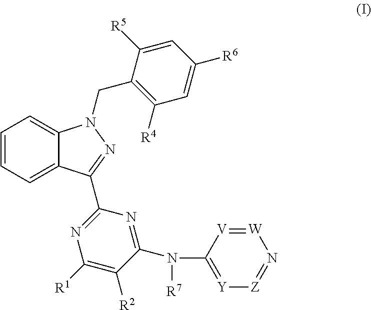

### FIELD OF APPLICATION OF THE INVENTION

The invention relates to substituted Benzyl Substituted Indazoles compounds, a process for their production and the use thereof.

### BACKGROUND OF THE INVENTION

One of the most fundamental characteristics of cancer cells is their ability to sustain chronic proliferation whereas in normal tissues the entry into and progression through the cell division cycle is tightly controlled to ensure a homeostasis of cell number and maintenance of normal tissue function. Loss of proliferation control was emphasized as one of the six hallmarks of cancer [Hanahan D and Weinberg R A, Cell 100, 57, 2000; Hanahan D and Weinberg R A, Cell 144, 646, 2011].

The eukaryotic cell division cycle (or cell cycle) ensures the duplication of the genome and its distribution to the daughter cells by passing through a coordinated and regulated sequence of events. The cell cycle is divided into four successive phases:

1. The G1 phase represents the time before the DNA replication, in which the cell grows and is sensitive to external stimuli.

2. In the S phase the cell replicates its DNA, and

3. in the G2 phase preparations are made for entry into mitosis.

4. In mitosis (M phase), the duplicated chromosomes get separated supported by a spindle device built from microtubules, and cell division into two daughter cells is completed.

To ensure the extraordinary high fidelity required for an accurate distribution of the chromosomes to the daughter cells, the passage through the cell cycle is strictly regulated and controlled. The enzymes that are necessary for the progression through the cycle must be activated at the correct time and are also turned off again as soon as the corresponding phase is passed. Corresponding control points (“checkpoints”) stop or delay the progression through the cell cycle if DNA damage is detected, or the DNA replication or the creation of the spindle device is not yet completed. The mitotic checkpoint (also known as spindle checkpoint or spindle assembly checkpoint) controls the accurate attachment of mircrotubules of the spindle device to the kinetochors (the attachment site for microtubules) of the duplicated chromosomes. The mitotic checkpoint is active as long as unattached kinetochores are present and generates a wait-signal to give the dividing cell the time to ensure that each kinetochore is attached to a spindle pole, and to correct attachment errors. Thus the mitotic checkpoint prevents a mitotic cell from completing cell division with unattached or erroneously attached chromosomes [Suijkerbuijk S J and Kops G J, Biochem. Biophys. Acta 1786, 24, 2008; Musacchio A and Salmon E D, Nat. Rev. Mol. Cell. Biol. 8, 379, 2007]. Once all kinetochores are attached with the mitotic spindle poles in a correct bipolar (amphitelic) fashion, the checkpoint is satisfied and the cell enters anaphase and proceeds through mitosis.

The mitotic checkpoint is established by a complex network of a number of essential proteins, including members of the MAD (mitotic arrest deficient, MAD 1-3) and Bub (Budding uninhibited by benzimidazole, Bub 1-3) families, Mps1 kinase, cdc20, as well as other components [reviewed in Bolanos-Garcia V M and Blundell T L, Trends Biochem. Sci. 36, 141, 2010], many of these being over-expressed in proliferating cells (e.g. cancer cells) and tissues [Yuan B et al., Clin. Cancer Res. 12, 405, 2006]. The major function of an unsatisfied mitotic checkpoint is to keep the anaphase-promoting complex/cyclosome (APC/C) in an inactive state. As soon as the checkpoint gets satisfied the APC/C ubiquitin-ligase targets cyclin B and securin for proteolytic degradation leading to separation of the paired chromosomes and exit from mitosis.

Inactive mutations of the Ser/Thr kinase Bub1 prevented the delay in progression through mitosis upon treatment of cells of the yeast S. cerevisiae with microtubule-destabilizing drugs, which led to the identification of Bub1 as a mitotic checkpoint protein [Roberts B T et al., Mol. Cell Biol., 14, 8282, 1994]. A number of recent publications provide evidence that Bub1 plays multiple roles during mitosis which, have been reviewed by Elowe [Elowe S, Mol. Cell. Biol. 31, 3085, 2011]. In particular, Bub1 is one of the first mitotic checkpoint proteins that binds to the kinetochores of duplicated chromosomes and probably acts as a scaffolding protein to constitute the mitotic checkpoint complex. Furthermore, via phosphorylation of histone H2A, Bub1 localizes the protein shugoshin to the centromeric region of the chromosomes to prevent premature segregation of the paired chromosomes [Kawashima et al. Science 327, 172, 2010]. In addition, together with a Thr-3 phosphorylated Histone H3 the shugoshin protein functions as a binding site for the chromosomal passenger complex which includes the proteins survivin, borealin, INCENP and Aurora B. The chromosomal passenger complex is seen as a tension sensor within the mitotic checkpoint mechanism, which dissolves erroneously formed microtubule-kinetochor attachments such as syntelic (both sister kinetochors are attached to one spindle pole) or merotelic (one kinetochor is attached to two spindle poles) attachments [Watanabe Y, Cold Spring Harb. Symp. Quant. Biol. 75, 419, 2010]. Recent data suggest that the phosphorylation of histone H2A at Thr 121 by Bub1 kinase is sufficient to localize AuroraB kinase to fulfill the attachment error correction checkpoint [Ricke et al. J. Cell Biol. 199, 931-949, 2012].

Incomplete mitotic checkpoint function has been linked with aneuploidy and tumourigenesis [Weaver B A and Cleveland D W, Cancer Res. 67, 10103, 2007; King R W, Biochim Biophys Acta 1786, 4, 2008]. In contrast, complete inhibition of the mitotic checkpoint has been recognised to result in severe chromosome missegregation and induction of apoptosis in tumour cells [Kops G J et al., Nature Rev. Cancer 5, 773, 2005; Schmidt M and Medema R H, Cell Cycle 5, 159, 2006; Schmidt M and Bastians H, Drug Res. Updates 10, 162, 2007]. Thus, mitotic checkpoint abrogation through pharmacological inhibition of components of the mitotic checkpoint, such as Bub1 kinase, represents a new approach for the treatment of proliferative disorders, including solid tumours such as carcinomas, sarcomas, leukaemias and lymphoid malignancies or other disorders, associated with uncontrolled cellular proliferation.

The present invention relates to chemical compounds that inhibit Bub1 kinase.

Established anti-mitotic drugs such as vinca alkaloids, taxanes or epothilones activate the mitotic checkpoint, inducing a mitotic arrest either by stabilising or destabilising microtubule dynamics. This arrest prevents separation of the duplicated chromosomes to form the two daughter cells. Prolonged arrest in mitosis forces a cell either into mitotic exit without cytokinesis (mitotic slippage or adaption) or into mitotic catastrophe leading to cell death [Rieder C L and Maiato H, Dev. Cell 7, 637, 2004]. In contrast, inhibitors of Bub1 prevent the establishment and/or functionality of the mitotic checkpoint and/or microtubule-kinetochor attachment error correction mechanisms, which finally results in severe chromosomal missegregation, induction of apoptosis and cell death.

These findings suggest that Bub1 inhibitors should be of therapeutic value for the treatment of proliferative disorders associated with enhanced uncontrolled proliferative cellular processes such as, for example, cancer, inflammation, arthritis, viral diseases, cardiovascular diseases, or fungal diseases in a warm-blooded animal such as man. WO 2013/050438, WO 2013/092512, WO 2013/167698 disclose substituted benzylindazoles, substituted benzylpyrazoles and substituted benzylcycloalkylpyrazoles, respectively, which are Bub1 kinase inhibitors.

Furthermore, WO 2014/147203, WO 2014/147204, WO2014202590, WO2014202588, WO2014202584, WO2014202583, and WO2015/063003, disclose substituted indazoles, substituted pyrazoles, and substituted cycloalkylpyrazoles, which are Bub1 kinase inhibitors.

Due to the fact that especially cancer disease as being expressed by uncontrolled proliferative cellular processes in tissues of different organs of the human- or animal body still is not considered to be a controlled disease in that sufficient drug therapies already exist, there is a strong need to provide further new therapeutically useful drugs, preferably inhibiting new targets and providing new therapeutic options (e.g. drugs with improved pharmacological properties, such as improved target Bub1 inhibition potency).

### DESCRIPTION OF THE INVENTION

Therefore, inhibitors of Bub1 represent valuable compounds that should complement therapeutic options either as single agents or in combination with other drugs.

In accordance with a first aspect, the invention relates to compounds of formula (I),

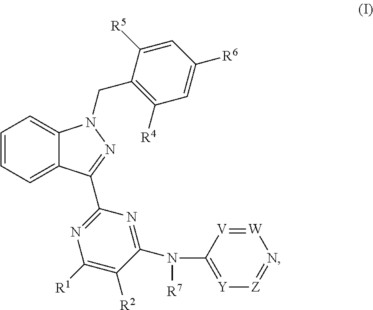

in which
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ and R² represent, independently of each other, hydrogen, halogen, or a group selected from:
   C₁-C₆-alkyl, C₃-C₆-cycloalkyl, C₁-C₆-haloalkyl, C₁-C₆-hydroxyalkyl,
   (C₁-C₃-alkoxy)-(C₁-C₃-alkyl)-, —(C₁-C₆-alkyl)-S—R¹⁴, —(C₁-C₆-alkyl)-S(═O)—R¹⁴,
   —(C₁-C₆-alkyl)-S(═O)₂—R¹⁴, —(C₁-C₆-alkyl)-S(═O)(═NR¹⁵)R¹⁴,
   —(C₁-C₆-alkyl)-N(R¹¹)R¹², —(C₁-C₆-alkyl)-N(R¹⁷)R¹⁸, 5- or 6-membered heteroaryl,
   a 5- to 7-membered oxygen containing heterocycloalkyl group,
   (1,3-dioxolan-2-yl)-(C₁-C₆-alkyl)-, (1,3-dioxan-2-yl)-(C₁-C₆-alkyl)-, an azetidinyl group, azetidinyl-(C₁-C₆-alkyl)-, a 5- to 7-membered heterocycloalkyl group, and (5- to 7-membered heterocycloalkyl)-(C₁-C₆-alkyl)-,
   in which 5- or 6-membered heteroaryl is connected to rest of the molecule via a carbon atom of the heteroaryl group,
   in which 5- to 7-membered oxygen containing heterocycloalkyl is connected to rest of the molecule via a carbon atom of the 5- to 7-membered oxygen containing heterocycloalkyl group,
   in which azetidinyl and 5- to 7-membered heterocycloalkyl are connected to the rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a substituent selected from: a hydroxy, a halogen atom, cyano, C₁-C₄-alkyl, C₁-C₄-haloalkyl, C₁-C₄-alkoxy, and C₁-C₄-haloalkoxy group,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₆-cycloalkyl, C₃-C₆-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₆-cycloalkyl,
      (C₃-C₄-cycloalkyl)methyl-C₃-C₆-cycloalkyloxy, —N(R¹¹)R¹², C(═O)OH and
      —N(H)C(═O)—(C₁-C₃-alkyl),
   wherein at least one of R¹ and R² represents a group which connects via a carbon atom of said group to the rest of the molecule,
   or,
   R¹ and R² together represent a group, which is selected from:

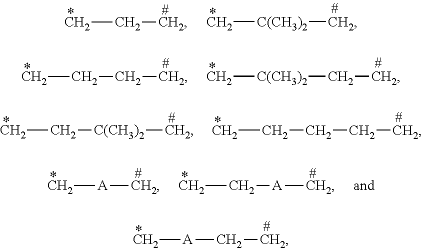

wherein A represents a group, which is selected from:
NH, N—(C₁-C₃-alkyl), N—(C₂-C₃-haloalkyl), N-benzyl, O, S, S(═O), S(═O)₂, and S(═O)(═NR¹⁵)R¹⁴,
and,
wherein * indicates the point of attachment of R¹ to the rest of the molecule,
and # indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₁-C₃-alkyl)-(C₁-C₃-alkoxy), —N(H)C(═O)-phenyl,
   —N(H)C(═O)—(C₃-C₄-cycloalkyl), —N(H)C(═O)—(C₁-C₃-hydroxyalkyl),
   —C(═O)OH, —C(═O)NH₂, —S(═O)₂—(C₁-C₃-alkyl),
   —N(H)C(═O)—(C₁-C₃-alkyl)-(C₃-C₄-cycloalkyl), and —N(H)C(═O)N(H)R¹⁹,
      said —N(H)C(═O)-phenyl being optionally substituted at the phenyl ring, one, two or three times, identically or differently, with a substituent selected from:
      halogen, hydroxy, cyano, C₁-C₄-alkyl, C₁-C₄-haloalkyl, C₁-C₄-alkoxy, C₁-C₄-haloalkoxy, C₃-C₆-cycloalkyl, and C₃-C₆-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R⁴ and R⁵ represent, independently of each other, hydrogen or fluorine,
R⁶ represents a group selected from:
   hydroxy, C₁-C₆-alkyl, C₁-C₆-haloalkyl, C₁-C₆-hydroxyalkyl,
   (C₁-C₃-alkoxy)-(C₁-C₆-alkyl)-, C₃-C₆-cycloalkyl, (C₃-C₆-cycloalkyl)-(C₁-C₃-alkyl) C₁-C₆-alkoxy, C₁-C₆-haloalkoxy, (C₂-C₆-hydroxyalkyl)-O—,
   (C₁-C₃-alkoxy)-(C₂-C₆-alkoxy)-, C₃-C₆-cycloalkyloxy,
   (C₃-C₆-cycloalkyl)-(C₁-C₃-alkoxy)-, and R⁸,
      wherein said hydroxyalkyl groups are optionally substituted with one, two or three halogen atoms selected from:
      fluorine, and chlorine,
R⁷ represents hydrogen, or a 2-hydroxyethyl group,
   or,
R² and R⁷ together represent a

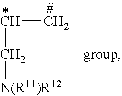

wherein * indicates the point of attachment of R² to the rest of the molecule,
and ^{#} indicates the point of attachment of R⁷ to the rest of the molecule,
R⁸ represents a group selected from:
   —(C₂-C₆-alkyl)-OC(═O)—C(H)(R⁹)—N(H)C(═O)—C(H)(R¹⁰)—NH₂, and
   —O—(C₂-C₆-alkyl)-OC(═O)—C(H)(R⁹)—N(H)C(═O)—C(H)(R¹⁰)—NH₂,
      wherein said C₂-C₆-alkyl groups are optionally substituted with one, two or three halogen atoms selected from:
      fluorine, and chlorine,
R⁹ and R¹⁰ independently of each other represent hydrogen (glycine) or a group selected from:
   CH₃ (alanine), C(H)(CH₃)₂ (valine), (CH₂)₂CH₃ (norvaline), CH₂C(H)(CH₃)₂ (leucine), C(H)(CH₃)CH₂CH₃ (isoleucine), (CH₂)₃CH₃ (norleucine), C(CH₃)₃ (2-tert-butylglycine), benzyl (phenylalanine), 4-hydroxybenzyl (tyrosine), (CH₂)₃NH₂ (ornithine), (CH₂)₄NH₂ (lysine), (CH₂)₂C(H)(OH)CH₂NH₂ (hydroxylysine), CH₂OH (serine), (CH₂)₂OH (homoserine), C(H)(OH)CH₃ (threonine),
   (CH₂)₃N(H)C(═NH)NH₂ (arginine), (CH₂)₃N(H)C(═O)NH₂ (citrulline), CH₂C(═O)NH₂ (asparagine), CH₂C(═O)OH (aspartic acid), (CH₂)₂C(═O)OH (glutamic acid),
   (CH₂)₂C(═O)NH₂ (glutamine), CH₂SH (cysteine), (CH₂)₂SH (homocysteine),
   (CH₂)₂SCH₃ (methionine), CH₂SCH₃ (S-methylcysteine), (1H-imidazol-4-yl)methyl-(histidine), (1H-indol-3-yl)methyl-(thryptophan), CH₂NH₂ (2,3-diaminopropanoic acid), and (CH₂)₂NH₂ (2,4-diaminobutanoic acid),
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl),
R¹⁴ represents, independently of each other, a group selected from:
   C₁-C₆-alkyl, C₃-C₆-cycloalkyl, phenyl, and benzyl,
      wherein said group is optionally substituted with one, two or three substituents, identical or different, selected from the group of hydroxy, halogen, and —NR¹¹R¹²,
R¹⁵ represents hydrogen or a group selected from:
   cyano, and —C(═O)R¹⁶
R¹⁶ represents a group selected from:
   C₁-C₆-alkyl, and C₁-C₆-haloalkyl,
R¹⁷ and R¹⁸ together with the nitrogen to which they are attached form
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₆-cycloalkyl, C₃-C₆-cycloalkyloxy,
      hydroxy, halogen, cyano, —C(═O)H, (C₁-C₄-alkyl)-C(═O)—, —N(R¹¹)R¹²,
      —(C₁-C₄-alkyl)-N(R¹¹)R¹², —C(═O)N(R¹¹)R¹², —(C₁-C₄-alkyl)-C(═O)N(R¹¹)R¹²,
      and —C(═O)OH,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₆-cycloalkyl, C₃-C₆-cycloalkyloxy,
      hydroxy, a halogen atom, cyano, —C(═O)H, (C₁-C₄-alkyl)-C(═O)—,
      —N(R¹¹)R¹², —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH,
R¹⁹ represents hydrogen or a group selected from:
   C₁-C₃-alkyl, C₁-C₃-haloalkyl, C₂-C₃-hydroxyalkyl, C₃-C₄-cycloalkyl,
   (C₃-C₄-cycloalkyl)-(C₁-C₃-alkyl) and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

In accordance with a variant of the first aspect of the invention, the invention relates to compounds of formula (I),

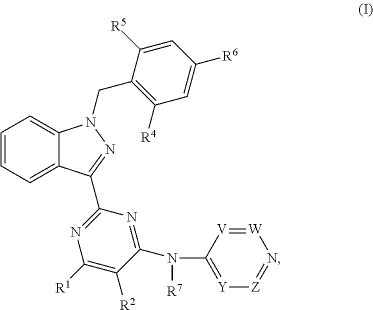

in which
V, W, Y and Z independently of each other represent CH or CR³,
or,
   V represents N, and W, Y and Z independently of each other represent CH or CR³,
or,
   W represents N, and V, Y and Z independently of each other represent CH or CR³,
or,
   V and Y represent N, and W and Z independently of each other represent CH or CR³,
   R¹ and R² represent, independently of each other, hydrogen, halogen, or a group selected from:
C₁-C₆-alkyl, C₁-C₆-haloalkyl, C₁-C₆-hydroxyalkyl, (C₁-C₃-alkoxy)-(C₁-C₃-alkyl)
—(C₁-C₆-alkyl)-S—R¹⁴, —(C₁-C₆-alkyl)-S(═O)—R¹⁴, —(C₁-C₆-alkyl)-S(═O)₂—R¹⁴,
—(C₁-C₆-alkyl)-S(═O)(═NR¹⁵)R¹⁴, —(C₁-C₆-alkyl)-N(R¹¹)R¹²,
—(C₁-C₆-alkyl)-N(R¹⁷)R¹⁸, (1,3-dioxolan-2-yl)-(C₁-C₆-alkyl)
(1,3-dioxan-2-yl)-(C₁-C₆-alkyl) an azetidinyl group, azetidinyl-(C₁-C₆-alkyl)
a 5- to 7-membered heterocycloalkyl group, and
(5- to 7-membered heterocycloalkyl)-(C₁-C₆-alkyl)-,
in which azetidinyl and 5- to 7-membered heterocycloalkyl are connected to the rest of the molecule via a carbon atom of the azetidinyl ring or via a carbon atom of the 5- to 7-membered heterocycloalkyl ring,
wherein azetidinyl is optionally substituted with a substituent selected from:
   hydroxy, a halogen atom, cyano, C₁-C₄-alkyl, C₁-C₄-haloalkyl,
   C₁-C₄-alkoxy, C₁-C₄-haloalkoxy, (C₁-C₃-alkoxy)-(C₁-C₄-alkyl)-,
   C₃-C₆-cycloalkyl, C₃-C₆-cycloalkyloxy, —N(R¹¹)R¹², and
   —N(H)C(═O)—(C₁-C₃-alkyl),
   or being substituted with two halogen atoms,
wherein 5- to 7-membered heterocycloalkyl is optionally substituted, one, two, three, four or five times, identically or differently, with a substituent selected from:
   hydroxy, halogen, cyano, C₁-C₄-alkyl, C₁-C₄-haloalkyl, C₁-C₄-alkoxy,
   C₁-C₄-haloalkoxy, (C₁-C₃-alkoxy)-(C₁-C₄-alkyl)-, C₃-C₆-cycloalkyl,
   C₃-C₆-cycloalkyloxy, —N(R¹¹)R¹², C(═O)OH and —N(H)C(═O)—(C₁-C₃-alkyl),
wherein at least one of R¹ and R² represents a group which connects via a carbon atom of said group to the rest of the molecule,
or,
R¹ and R² together represent a group, which is selected from:

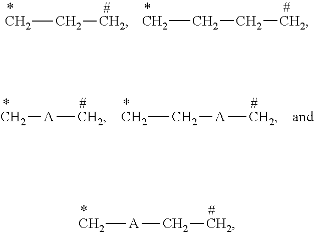

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and ^{#} indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₁-C₃-alkyl)-(C₁-C₃-alkoxy), —N(H)C(═O)-phenyl,
   —N(H)C(═O)—(C₃-C₄-cycloalkyl), —N(H)C(═O)—(C₁-C₃-hydroxyalkyl), —C(═O)OH,
   —C(═O)NH₂, —S(═O)₂—(C₁-C₃-alkyl), —N(H)C(═O)—(C₁-C₃-alkyl)-(C₃-C₄-cycloalkyl), and
   —N(H)C(═O)N(H)R¹⁹,
      said —N(H)C(═O)-phenyl being optionally substituted at the phenyl ring, one, two or three times, identically or differently, with a substituent selected from:
      halogen, hydroxy, cyano, C₁-C₄-alkyl, C₁-C₄-haloalkyl, C₁-C₄-alkoxy,
      C₁-C₄-haloalkoxy, C₃-C₆-cycloalkyl, and C₃-C₆-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R⁴ and R⁵ represent, independently of each other, hydrogen or fluorine,
R⁶ represents a group selected from:
   hydroxy, C₁-C₆-alkyl, C₁-C₆-haloalkyl, C₁-C₆-hydroxyalkyl,
   (C₁-C₃-alkoxy)-(C₁-C₆-alkyl)-, C₃-C₆-cycloalkyl, (C₃-C₆-cycloalkyl)-(C₁-C₃-alkyl) C₁-C₆-alkoxy, C₁-C₆-haloalkoxy, (C₂-C₆-hydroxyalkyl)-O—,
   (C₁-C₃-alkoxy)-(C₂-C₆-alkoxy)-, C₃-C₆-cycloalkyloxy,
   (C₃-C₆-cycloalkyl)-(C₁-C₃-alkoxy)-, and R⁸,
      wherein said hydroxyalkyl groups are optionally substituted with one, two or three halogen atoms selected from:
      fluorine, and chlorine,
R⁷ represents hydrogen, or a 2-hydroxyethyl group,
   or,
R² and R⁷ together represent a

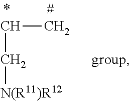

wherein * indicates the point of attachment of R² to the rest of the molecule,
and ^{#} indicates the point of attachment of R⁷ to the rest of the molecule,
R⁸ represents a group selected from:
   —(C₂-C₆-alkyl)-OC(═O)—C(H)(R⁹)—N(H)C(═O)—C(H)(R¹⁰)—NH₂, and
   —O—(C₂-C₆-alkyl)-OC(═O)—C(H)(R⁹)—N(H)C(═O)—C(H)(R¹⁰)—NH₂,
      wherein said C₂-C₆-alkyl groups are optionally substituted with one, two or three halogen atoms selected from:
      fluorine, and chlorine,
R⁹ and R¹⁰ independently of each other represent hydrogen (glycine) or a group selected from:
   CH₃ (alanine), C(H)(CH₃)₂ (valine), (CH₂)₂CH₃ (norvaline), CH₂C(H)(CH₃)₂ (leucine), C(H)(CH₃)CH₂CH₃ (isoleucine), (CH₂)₃CH₃ (norleucine), C(CH₃)₃ (2-tert-butylglycine), benzyl (phenylalanine), 4-hydroxybenzyl (tyrosine), (CH₂)₃NH₂ (ornithine), (CH₂)₄NH₂ (lysine), (CH₂)₂C(H)(OH)CH₂NH₂ (hydroxylysine), CH₂OH (serine), (CH₂)₂OH (homoserine), C(H)(OH)CH₃ (threonine),
   (CH₂)₃N(H)C(═NH)NH₂ (arginine), (CH₂)₃N(H)C(═O)NH₂ (citrulline), CH₂C(═O)NH₂ (asparagine), CH₂C(═O)OH (aspartic acid), (CH₂)₂C(═O)OH (glutamic acid),
   (CH₂)₂C(═O)NH₂ (glutamine), CH₂SH (cysteine), (CH₂)₂SH (homocysteine),
   (CH₂)₂SCH₃ (methionine), CH₂SCH₃ (S-methylcysteine), (1H-imidazol-4-yl)methyl-(histidine), (1H-indol-3-yl)methyl-(thryptophan), CH₂NH₂ (2,3-diaminopropanoic acid), and (CH₂)₂NH₂ (2,4-diaminobutanoic acid),
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl),
R¹⁴ represents, independently of each other, a group selected from:
   C₁-C₆-alkyl, C₃-C₆-cycloalkyl, phenyl, and benzyl,
      wherein said group is optionally substituted with one, two or three substituents, identical or different, selected from the group of hydroxy, halogen, and —NR¹¹R¹²,
R¹⁵ represents hydrogen or a group selected from:
   cyano, and —C(═O)R¹⁶
R¹⁶ represents a group selected from:
   C₁-C₆-alkyl, and C₁-C₆-haloalkyl,
R¹⁷ and R¹⁸ together with the nitrogen to which they are attached form
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₆-cycloalkyl, C₃-C₆-cycloalkyloxy,
      hydroxy, halogen, cyano, —C(═O)H, (C₁-C₄-alkyl)-C(═O)—, —N(R¹¹)R¹²,
      —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH, or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₆-cycloalkyl, C₃-C₆-cycloalkyloxy,
      hydroxy, a halogen atom, cyano, —C(═O)H, (C₁-C₄-alkyl)-C(═O)—,
      —N(R¹¹)R¹², —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH,
R¹⁹ represents hydrogen or a group selected from:
   C₁-C₃-alkyl, C₁-C₃-haloalkyl, C₂-C₃-hydroxyalkyl, C₃-C₄-cycloalkyl,
   (C₃-C₄-cycloalkyl)-(C₁-C₃-alkyl) and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

A second aspect of the invention are compounds of formula (I) as defined herein, wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ and R² represent, independently of each other hydrogen, halogen, or a group selected from:
   C₁-C₅-alkyl, C₃-C₆-cycloalkyl, C₁-C₅-haloalkyl, C₁-C₅-hydroxyalkyl,
   (C₁-C₃-alkoxy)-(C₁-C₃-alkyl)-, —(C₁-C₅-alkyl)-S—R¹⁴, —(C₁-C₅-alkyl)-S(═O)—R¹⁴,
   —(C₁-C₅-alkyl)-S(═O)₂—R¹⁴, —(C₁-C₅-alkyl)-S(═O)(═NR¹⁵)R¹⁴,
   —(C₁-C₅-alkyl)-N(R¹¹)R¹², —(C₁-C₅-alkyl)-N(R¹⁷)R¹⁸, 5- or 6-membered heteroaryl,
   a 5- to 7-membered oxygen containing heterocycloalkyl group,
   (1,3-dioxolan-2-yl)-(C₁-C₅-alkyl)-, (1,3-dioxan-2-yl)-(C₁-C₅-alkyl)-, an azetidinyl group, azetidinyl-(C₁-C₅-alkyl)-, a 5- to 7-membered heterocycloalkyl group, and
   (5- to 7-membered heterocycloalkyl)-(C₁-C₅-alkyl)-,
   in which 5- or 6-membered heteroaryl is connected to rest of the molecule via a carbon atom of the heteroaryl group,
   in which 5- to 7-membered oxygen containing heterocycloalkyl is connected to rest of the molecule via a carbon atom of the 5- to 7-membered oxygen containing heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a methyl group,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₄-cycloalkyl, C₃-C₄-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₄-cycloalkyl,
      (C₃-C₄-cycloalkyl)methyl-C₃-C₄-cycloalkyloxy, —N(R¹¹)R¹², —C(═O)OH and —N(H)C(═O)—(C₁-C₃-alkyl),
   wherein at least one of R¹ and R² represents a group which connects via a carbon atom of said group to the rest of the molecule,
   or,
   R¹ and R² together represent a group, which is selected from:

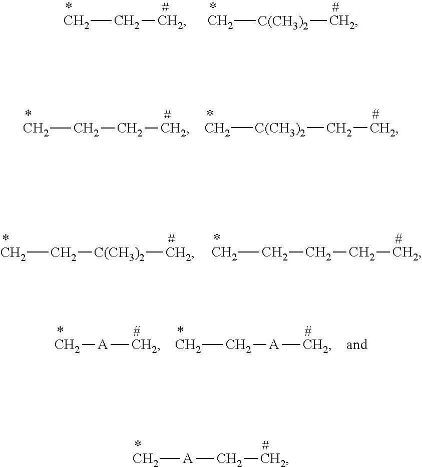

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule, and # indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₁-C₃-alkyl)-(C₁-C₃-alkoxy), —N(H)C(═O)-phenyl,
   —N(H)C(═O)—(C₃-C₄-cycloalkyl), —N(H)C(═O)—(C₁-C₃-hydroxyalkyl),
   —C(═O)OH, —C(═O)NH₂, —S(═O)₂—(C₁-C₃-alkyl),
   —N(H)C(═O)—(C₁-C₃-alkyl)-(C₃-C₄-cycloalkyl), and —N(H)C(═O)N(H)R¹⁹,
      said —N(H)C(═O)-phenyl being optionally substituted at the phenyl ring, one, two or three times, identically or differently, with a substituent selected from: a halogen atom, hydroxy, cyano, C₁-C₄-alkyl, C₁-C₄-haloalkyl,
      C₁-C₄-alkoxy, C₁-C₄-haloalkoxy, C₃-C₄-cycloalkyl, and
      C₃-C₄-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R⁴ and R⁵ represent, independently of each other, hydrogen or fluorine,
R⁶ represents a group selected from:
   hydroxy, C₁-C₄-alkyl, C₁-C₄-haloalkyl, C₁-C₄-hydroxyalkyl,
   (C₁-C₃-alkoxy)-(C₁-C₃-alkyl)-, C₃-C₄-cycloalkyl, (C₃-C₄-cycloalkyl)-(C₁-C₃-alkyl) C₁-C₄-alkoxy, C₁-C₄-haloalkoxy, (C₂-C₄-hydroxyalkyl)-O—,
   (C₁-C₃-alkoxy)-(C₂-C₃-alkoxy)-, C₃-C₄-cycloalkyloxy,
   (C₃-C₄-cycloalkyl)-(C₁-C₃-alkoxy)-, and R⁸,
      wherein said hydroxyalkyl groups are optionally substituted with one, two or three halogen atoms selected from:
      fluorine, and chlorine,
R⁷ represents hydrogen, or a 2-hydroxyethyl group,
   or,
R² and R⁷ together represent a

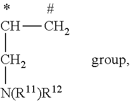

wherein * indicates the point of attachment of R² to the rest of the molecule,
and ^{#} indicates the point of attachment of R⁷ to the rest of the molecule,
R⁸ represents a group selected from:
   —(C₂-C₄-alkyl)-OC(═O)—C(H)(R⁹)—N(H)C(═O)—C(H)(R¹⁰)—NH₂, and
   —O—(C₂-C₄-alkyl)-OC(═O)—C(H)(R⁹)—N(H)C(═O)—C(H)(R¹⁰)—NH₂,
      wherein said C₂-C₄-alkyl groups are optionally substituted with one, two or three halogen atoms selected from:
      fluorine, and chlorine,
R⁹ and R¹⁰ independently of each other represent hydrogen (glycine) or a group selected from:
   CH₃ (alanine), C(H)(CH₃)₂ (valine), (CH₂)₂CH₃ (norvaline), CH₂C(H)(CH₃)₂ (leucine), C(H)(CH₃)CH₂CH₃ (isoleucine), (CH₂)₃CH₃ (norleucine), C(CH₃)₃ (2-tert-butylglycine), benzyl (phenylalanine), 4-hydroxybenzyl (tyrosine),
   (CH₂)₃NH₂ (ornithine), (CH₂)₄NH₂ (lysine), (CH₂)₂C(H)(OH)CH₂NH₂ (hydroxylysine),
   CH₂OH (serine), (CH₂)₂OH (homoserine), C(H)(OH)CH₃ (threonine),
   (CH₂)₃N(H)C(═NH)NH₂ (arginine), (CH₂)₃N(H)C(═O)NH₂ (citrulline), CH₂C(═O)NH₂ (asparagine), CH₂C(═O)OH (aspartic acid), (CH₂)₂C(═O)OH (glutamic acid),
   (CH₂)₂C(═O)NH₂ (glutamine), CH₂SH (cysteine), (CH₂)₂SH (homocysteine),
   (CH₂)₂SCH₃ (methionine), CH₂SCH₃ (S-methylcysteine), (1H-imidazol-4-yl)methyl-(histidine),
   (1H-indol-3-yl)methyl-(thryptophan), CH₂NH₂ (2,3-diaminopropanoic acid), and
   (CH₂)₂NH₂ (2,4-diaminobutanoic acid),
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl),
R¹⁴ represents, independently of each other, a group selected from:
   C₁-C₄-alkyl, C₃-C₄-cycloalkyl, phenyl, and benzyl,
      wherein said group is optionally substituted with one, two or three substituents, identical or different, selected from the group of hydroxy, halogen, and —NR¹¹R¹²,
R¹⁵ represents hydrogen or a group selected from:
   cyano, and —C(═O)R¹⁶
R¹⁶ represents a group selected from:
   C₁-C₃-alkyl, and C₁-C₃-haloalkyl,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₄-cycloalkyl, C₃-C₄-cycloalkyloxy,
      hydroxy, halogen, cyano, —C(═O)H, (C₁-C₄-alkyl)-C(═O)—, —N(R¹¹)R¹²,
      —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₄-cycloalkyl, C₃-C₄-cycloalkyloxy,
      hydroxy, a halogen atom, cyano, —C(═O)H, (C₁-C₄-alkyl)-C(═O)—,
      —N(R¹¹)R¹², —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH,
R¹⁹ represents hydrogen or a group selected from:
   C₁-C₃-alkyl, C₁-C₃-haloalkyl, C₂-C₃-hydroxyalkyl, C₃-C₄-cycloalkyl,
   (C₃-C₄-cycloalkyl)-(C₁-C₃-alkyl)-, and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

In accordance with a variant of the second aspect of the invention, the invention relates to compounds of formula (I) as defined herein,

wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ and R² represent, independently of each other hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₆-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, (1,3-dioxolan-2-yl)-(C₁-C₅-alkyl)-,
   (1,3-dioxan-2-yl)-(C₁-C₅-alkyl)-, an azetidinyl group, azetidinyl-(C₁-C₅-alkyl)-,
   a 5- to 7-membered heterocycloalkyl group, and
   (5- to 7-membered heterocycloalkyl)-(C₁-C₅-alkyl)-,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₄-cycloalkyl, C₃-C₄-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₄-cycloalkyl,
      C₃-C₄-cycloalkyloxy, —N(R¹¹)R¹², —C(═O)OH and —N(H)C(═O)—(C₁-C₃-alkyl),
   wherein at least one of R¹ and R² represents a group which connects via a carbon atom of said group to the rest of the molecule,
   or,

R¹ and R² together represent a group, which is selected from:

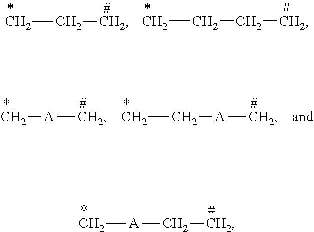

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and ^{#} indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₁-C₃-alkyl)-(C₁-C₃-alkoxy), —N(H)C(═O)-phenyl,
   —N(H)C(═O)—(C₃-C₄-cycloalkyl), —N(H)C(═O)—(C₁-C₃-hydroxyalkyl), —C(═O)OH,
   —C(═O)NH₂, —S(═O)₂—(C₁-C₃-alkyl), —N(H)C(═O)—(C₁-C₃-alkyl)-(C₃-C₄-cycloalkyl), and
   —N(H)C(═O)N(H)R¹⁹,
      said —N(H)C(═O)-phenyl being optionally substituted at the phenyl ring, one, two or three times, identically or differently, with a substituent selected from: a halogen atom, hydroxy, cyano, C₁-C₄-alkyl, C₁-C₄-haloalkyl,
      C₁-C₄-alkoxy, C₁-C₄-haloalkoxy, C₃-C₄-cycloalkyl, and
      C₃-C₄-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R⁴ and R⁵ represent, independently of each other, hydrogen or fluorine,
R⁶ represents a group selected from:
   hydroxy, C₁-C₄-alkyl, C₁-C₄-haloalkyl, C₁-C₄-hydroxyalkyl,
   (C₁-C₃-alkoxy)-(C₁-C₃-alkyl)-, C₃-C₄-cycloalkyl, (C₃-C₄-cycloalkyl)-(C₁-C₃-alkyl) C₁-C₄-alkoxy, C₁-C₄-haloalkoxy, (C₂-C₄-hydroxyalkyl)-O—,
   (C₁-C₃-alkoxy)-(C₂-C₃-alkoxy)-, C₃-C₄-cycloalkyloxy,
   (C₃-C₄-cycloalkyl)-(C₁-C₃-alkoxy)-, and R⁸,
      wherein said hydroxyalkyl groups are optionally substituted with one, two or three halogen atoms selected from:
      fluorine, and chlorine,
R⁷ represents hydrogen, or a 2-hydroxyethyl group,
   or,
R² and R⁷ together represent a

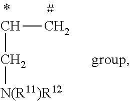

wherein * indicates the point of attachment of R² to the rest of the molecule,
and ^{#} indicates the point of attachment of R⁷ to the rest of the molecule,
R⁸ represents a group selected from:
   —(C₂-C₄-alkyl)-OC(═O)—C(H)(R⁹)—N(H)C(═O)—C(H)(R¹⁰)—NH₂, and
   —O—(C₂-C₄-alkyl)-OC(═O)—C(H)(R⁹)—N(H)C(═O)—C(H)(R¹⁰)—NH₂,
      wherein said C₂-C₄-alkyl groups are optionally substituted with one, two or three halogen atoms selected from:
      fluorine, and chlorine,
R⁹ and R¹⁰ independently of each other represent hydrogen (glycine) or a group selected from:
   CH₃ (alanine), C(H)(CH₃)₂ (valine), (CH₂)₂CH₃ (norvaline), CH₂C(H)(CH₃)₂ (leucine), C(H)(CH₃)CH₂CH₃ (isoleucine), (CH₂)₃CH₃ (norleucine), C(CH₃)₃ (2-tert-butylglycine), benzyl (phenylalanine), 4-hydroxybenzyl (tyrosine),
   (CH₂)₃NH₂ (ornithine), (CH₂)₄NH₂ (lysine), (CH₂)₂C(H)(OH)CH₂NH₂ (hydroxylysine),
   CH₂OH (serine), (CH₂)₂OH (homoserine), C(H)(OH)CH₃ (threonine),
   (CH₂)₃N(H)C(═NH)NH₂ (arginine), (CH₂)₃N(H)C(═O)NH₂ (citrulline), CH₂C(═O)NH₂ (asparagine), CH₂C(═O)OH (aspartic acid), (CH₂)₂C(═O)OH (glutamic acid),
   (CH₂)₂C(═O)NH₂ (glutamine), CH₂SH (cysteine), (CH₂)₂SH (homocysteine),
   (CH₂)₂SCH₃ (methionine), CH₂SCH₃ (S-methylcysteine), (1H-imidazol-4-yl)methyl-(histidine),
   (1H-indol-3-yl)methyl-(thryptophan), CH₂NH₂ (2,3-diaminopropanoic acid), and
   (CH₂)₂NH₂ (2,4-diaminobutanoic acid),
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl),
R¹⁴ represents, independently of each other, a group selected from:
   C₁-C₄-alkyl, C₃-C₄-cycloalkyl, phenyl, and benzyl,
      wherein said group is optionally substituted with one, two or three substituents, identical or different, selected from the group of hydroxy, halogen, and —NR¹¹R¹²,
R¹⁵ represents hydrogen or a group selected from:
   cyano, and —C(═O)R¹⁶
R¹⁶ represents a group selected from:
   C₁-C₃-alkyl, and C₁-C₃-haloalkyl,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₄-cycloalkyl, C₃-C₄-cycloalkyloxy,
      hydroxy, halogen, cyano, —C(═O)H, (C₁-C₄-alkyl)-C(═O)—, —N(R¹¹)R¹²,
      —(C₁-C₄-alkyl)-N(R¹¹)R¹², —C(═O)N(R¹¹)R¹², —(C₁-C₄-alkyl)-C(═O)N(R¹¹)R¹², and —C(═O)OH,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₄-cycloalkyl, C₃-C₄-cycloalkyloxy,
      hydroxy, a halogen atom, cyano, —C(═O)H, (C₁-C₄-alkyl)-C(═O)—,
      —N(R¹¹)R¹², —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH,
R¹⁹ represents hydrogen or a group selected from:
   C₁-C₃-alkyl, C₁-C₃-haloalkyl, C₂-C₃-hydroxyalkyl, C₃-C₄-cycloalkyl,
   (C₃-C₄-cycloalkyl)-(C₁-C₃-alkyl) and (C₁-C₃-alkoxy)-(C₂-C₃-alkyl)-,
      or an N-oxide, a salt, a tautomer or a stereoisomer of said compound, or a salt of said N-oxide, tautomer or stereoisomer.

A third aspect of the invention are compounds of formula (I) as defined herein, wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ represents hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, 5-membered heteroaryl, and a 5- or 6-membered oxygen containing heterocycloalkyl group,
   in which 5-membered heteroaryl is connected to rest of the molecule via a carbon atom of the heteroaryl group,
   wherein 5-membered heteroaryl is optionally substituted, one or two times, with a methyl group,
R² represents hydrogen, halogen, or a group selected from:
   C₁-C₅-alkyl, C₃-C₆-cycloalkyl, C₁-C₅-haloalkyl, C₁-C₅-hydroxyalkyl,
   (C₁-C₃-alkoxy)-(C₁-C₃-alkyl)-, —(C₁-C₅-alkyl)-S—R¹⁴, —(C₁-C₅-alkyl)-S(═O)—R¹⁴,
   —(C₁-C₅-alkyl)-S(═O)₂—R¹⁴, —(C₁-C₅-alkyl)-S(═O)(═NR¹⁵)R¹⁴,
   —(C₁-C₅-alkyl)-N(R¹¹)R¹², —(C₁-C₅-alkyl)-N(R¹⁷)R¹⁸,
   (1,3-dioxolan-2-yl)-(C₁-C₅-alkyl)-, (1,3-dioxan-2-yl)-(C₁-C₅-alkyl)-, an azetidinyl group, azetidinyl-(C₁-C₅-alkyl)-, a 5- to 7-membered heterocycloalkyl group, and
   (5- to 7-membered heterocycloalkyl)-(C₁-C₅-alkyl)-,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      a halogen atom, C₁-C₄-alkyl, and C₁-C₄-haloalkyl,
      or being substituted with two halogen atoms,
   wherein 5- to 7-membered heterocycloalkyl is optionally substituted, one, two, three, four or five times, identically or differently, with a substituent selected from:
      halogen, C₁-C₄-alkyl, C₁-C₄-haloalkyl and cyclopropylmethyl-,
   wherein at least one of R¹ and R² represents a group which connects via a carbon atom of said group to the rest of the molecule,
   or,
   R¹ and R² together represent a group, which is selected from:

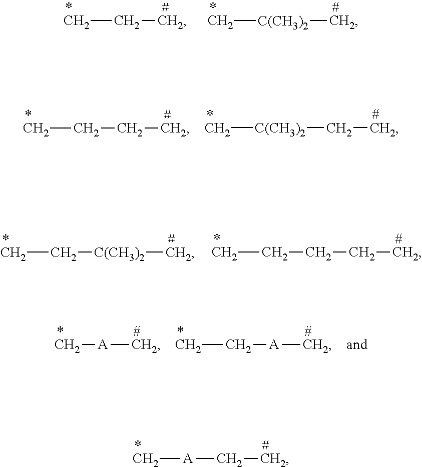

wherein A represents a group, which is selected from:
NH, N—(C₁-C₃-alkyl), N—(C₂-C₃-haloalkyl), N-benzyl, O, S, S(═O), and S(═O)₂,
and,
wherein * indicates the point of attachment of R¹ to the rest of the molecule,
and ^{#} indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy, C₁-C₃-haloalkoxy, —N(H)C(═O)H, —N(H)C(═O)—(C₁-C₃-alkyl), —N(H)C(═O)—(C₃-C₄-cycloalkyl), —N(H)C(═O)—(C₁-C₃-hydroxyalkyl), —C(═O)OH, —C(═O)NH₂, and —S(═O)₂—(C₁-C₃-alkyl),
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R⁴ and R⁵ represent, independently of each other, hydrogen or fluorine,
R⁶ represents a group selected from:
   hydroxy, C₁-C₄-alkoxy, C₁-C₄-haloalkoxy, (C₂-C₄-hydroxyalkyl)-O—,
   (C₁-C₃-alkoxy)-(C₂-C₃-alkoxy)-, and R⁸,
R⁷ represents hydrogen, or a 2-hydroxyethyl group,
   or,
R² and R⁷ together represent a

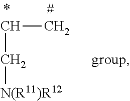

wherein * indicates the point of attachment of R² to the rest of the molecule,
and # indicates the point of attachment of R⁷ to the rest of the molecule,
R⁸ represents a group selected from:
   —(C₂-C₄-alkyl)-OC(═O)—C(H)(R⁹)—N(H)C(═O)—C(H)(R¹⁰)—NH₂, and
   —O—(C₂-C₄-alkyl)-OC(═O)—C(H)(R⁹)—N(H)C(═O)—C(H)(R¹⁰)—NH₂,
R⁹ and R¹⁰ independently of each other represent a group selected from:
   CH₃ (alanine), C(H)(CH₃)₂ (valine), (CH₂)₂CH₃ (norvaline), (CH₂)₃NH₂ (ornithine),
   (CH₂)₄NH₂ (lysine), and (CH₂)₃N(H)C(═NH)NH₂ (arginine),
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl),
R¹⁴ represents, independently of each other, a group selected from:
   C₁-C₄-alkyl, C₃-C₄-cycloalkyl,
R¹⁵ represents hydrogen or a group selected from:
   cyano, and —C(═O)R¹⁶
R¹⁶ represents a group selected from:
   C₁-C₃-alkyl, and C₁-C₃-haloalkyl,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, halogen,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, and a halogen atom,
         or an N-oxide, a salt, a tautomer or a stereoisomer of said compound, or a salt of said N-oxide, tautomer or stereoisomer.

In accordance with a variant of the third aspect of the invention, the invention relates to compounds of formula (I) as defined herein, wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ represents hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹², and
   —(C₁-C₅-alkyl)-N(R¹⁷)R¹⁸,
R² represents hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₃-alkyl)-N(R¹¹)R¹²,
   —(C₁-C₅-alkyl)-N(R¹⁷)R¹⁸, (1,3-dioxolan-2-yl)-(C₁-C₃-alkyl)-,
   (1,3-dioxan-2-yl)-(C₁-C₃-alkyl)-, an azetidinyl group, azetidinyl-(C₁-C₃-alkyl)-,
   a 5- to 7-membered heterocycloalkyl group, and
   (5- to 7-membered heterocycloalkyl)-(C₁-C₃-alkyl)-,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      a halogen atom, C₁-C₄-alkyl, and C₁-C₄-haloalkyl,
      or being substituted with two halogen atoms,
   wherein 5- to 7-membered heterocycloalkyl is optionally substituted, one, two, three, four or five times, identically or differently, with a substituent selected from:
      halogen, C₁-C₄-alkyl, and C₁-C₄-haloalkyl,
   wherein at least one of R¹ and R² represents a group which connects via a carbon atom of said group to the rest of the molecule,
   or,

R¹ and R² together represent a group, which is selected from:

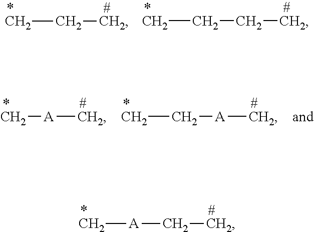

wherein A represents a group, which is selected from:
NH, N—(C₁-C₃-alkyl), N—(C₂-C₃-haloalkyl), N-benzyl, O, S, S(═O), and S(═O)₂, and,
wherein * indicates the point of attachment of R¹ to the rest of the molecule,
and ^{#} indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₃-C₄-cycloalkyl), —N(H)C(═O)—(C₁-C₃-hydroxyalkyl), —C(═O)OH,
   —C(═O)NH₂, and —S(═O)₂—(C₁-C₃-alkyl),
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy,
R⁴ and R⁵ represent, independently of each other, hydrogen or fluorine,
R⁶ represents a group selected from:
   hydroxy, C₁-C₄-alkoxy, C₁-C₄-haloalkoxy, (C₂-C₄-hydroxyalkyl)-O—,
   (C₁-C₃-alkoxy)-(C₂-C₃-alkoxy)-, and R⁸,
R⁷ represents hydrogen, or a 2-hydroxyethyl group,
   or,
R² and R⁷ together represent a

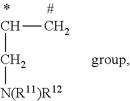

wherein * indicates the point of attachment of R² to the rest of the molecule,
and ^{#} indicates the point of attachment of R⁷ to the rest of the molecule,
R⁸ represents a group selected from:
   —(C₂-C₄-alkyl)-OC(═O)—C(H)(R⁹)—N(H)C(═O)—C(H)(R¹⁰)—NH₂, and
   —O—(C₂-C₄-alkyl)-OC(═O)—C(H)(R⁹)—N(H)C(═O)—C(H)(R¹⁰)—NH₂,
R⁹ and R¹⁰ independently of each other represent a group selected from:
   CH₃ (alanine), C(H)(CH₃)₂ (valine), (CH₂)₂CH₃ (norvaline), (CH₂)₃NH₂ (ornithine),
   (CH₂)₄NH₂ (lysine), and (CH₂)₃N(H)C(═NH)NH₂ (arginine),
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl),
R¹⁴ represents, independently of each other, a group selected from:
   C₁-C₄-alkyl, C₃-C₄-cycloalkyl,
R¹⁵ represents hydrogen or a group selected from:
   cyano, and —C(═O)R¹⁶
R¹⁶ represents a group selected from:
   C₁-C₃-alkyl, and C₁-C₃-haloalkyl,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, halogen,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, and a halogen atom,
         or an N-oxide, a salt, a tautomer or a stereoisomer of said compound, or a salt of said N-oxide, tautomer or stereoisomer.

A fourth aspect of the invention are compounds of formula (I) as defined herein, wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ represents hydrogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)₂—R¹⁴, —(C₁-C₅-alkyl)-N(R¹⁷)R¹⁸,
   5-membered heteroaryl, and a 5- or 6-membered oxygen containing heterocycloalkyl group,
   in which 5-membered heteroaryl is connected to rest of the molecule via a carbon atom of the heteroaryl group,
   wherein 5-membered heteroaryl is optionally substituted, one or two times, with a methyl group,
R² represents hydrogen, halogen, or a group selected from:
   C₁-C₅-alkyl, cyclopropyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, (1,3-dioxolan-2-yl)-(C₁-C₅-alkyl)-, an azetidinyl group,
   azetidinyl-(C₁-C₅-alkyl)-, and a 5- to 7-membered heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      C₁-C₄-haloalkyl,
   wherein 5- to 7-membered heterocycloalkyl is optionally substituted with a substituent selected from:
      C₁-C₄-haloalkyl and cyclopropylmethyl-,
   wherein at least one of R¹ and R² represents a group which connects via a carbon atom of said group to the rest of the molecule,
   or,
   R¹ and R² together represent a group, which is selected from:

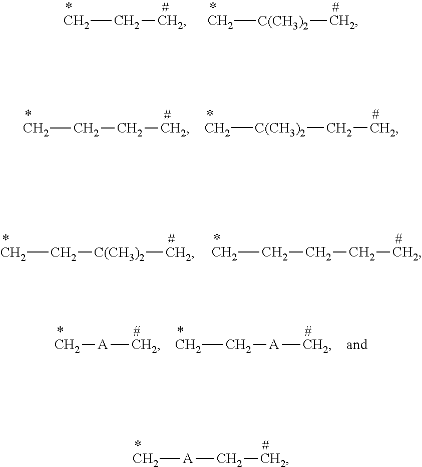

wherein A represents a group, which is selected from:
NH, N—(C₁-C₃-alkyl), N-(2,2,2-trifluoroethyl), N-benzyl, O, S, and S(═O)₂,
and,
wherein * indicates the point of attachment of R¹ to the rest of the molecule,
and ^{#} indicates the point of attachment of R² to the rest of the molecule,
R³ represents, independently of each other, halogen or a group selected from: hydroxy, C₁-C₃-alkyl, C₃-C₄-cycloalkyl, C₁-C₃-haloalkyl, C₁-C₃-alkoxy, C₁-C₃-haloalkoxy, —N(H)C(═O)—(C₁-C₃-alkyl), —N(H)C(═O)—(C₃-C₄-cycloalkyl), —C(═O)OH, —C(═O)NH₂, and —S(═O)₂—(C₁-C₃-alkyl),
R⁴ and R⁵ represent, independently of each other, hydrogen or fluorine,
R⁶ represents a group selected from:
   hydroxy, C₁-C₄-alkoxy, (C₂-C₄-hydroxyalkyl)-O—, and
   (C₁-C₃-alkoxy)-(C₂-C₃-alkoxy)-,
R⁷ represents hydrogen, or a 2-hydroxyethyl group,
   or,
R² and R⁷ together represent a

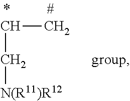

wherein * indicates the point of attachment of R² to the rest of the molecule,
and ^{#} indicates the point of attachment of R⁷ to the rest of the molecule,
R¹¹ and R¹² independently of each other represents hydrogen or a group selected from:
   C₂-C₃-haloalkyl, and —C(═O)H,
R¹⁴ represents, independently of each other, a group selected from:
   C₁-C₄-alkyl,
R¹⁵ represents hydrogen or a group selected from:
   —C(═O)R¹⁶
R¹⁶ represents a group selected from:
   C₁-C₃-haloalkyl,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom selected from 0,
   said azetidinyl group being substituted with two halogen atoms,
   said 5- to 7-membered heterocycloalkyl group being optionally substituted, two times, identically or differently, with a substituent selected from:
      halogen atom,
         or an N-oxide, a salt, a tautomer or a stereoisomer of said compound, or a salt of said N-oxide, tautomer or stereoisomer.

In accordance with a variant of the fourth aspect of the invention, the invention relates to compounds of formula (I) as defined herein,

wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z independently of each other represent CH or CR³,
R¹ represents hydrogen, or a group selected from:
   C₁-C₅-alkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)₂—R¹⁴, and —(C₁-C₅-alkyl)-N(R¹⁷)R¹⁸,
R² represents hydrogen, halogen, or a group selected from:
   C₁-C₅-alkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, (1,3-dioxolan-2-yl)-(C₁-C₅-alkyl)-, an azetidinyl group,
   azetidinyl-(C₁-C₅-alkyl)-, and a 5- to 7-membered heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      C₁-C₄-haloalkyl,
   wherein 5- to 7-membered heterocycloalkyl is optionally substituted with a substituent selected from:
      C₁-C₄-haloalkyl,
   wherein at least one of R¹ and R² represents a group which connects via a carbon atom of said group to the rest of the molecule,
   or,
   R¹ and R² together represent a group, which is selected from:

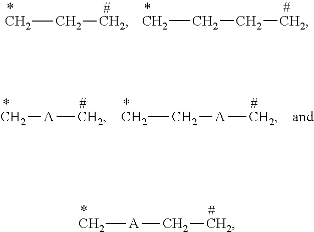

wherein A represents a group, which is selected from:
NH, N—(C₁-C₃-alkyl), N-benzyl, O, S, and S(═O)₂,
and,
wherein * indicates the point of attachment of R¹ to the rest of the molecule,
and ^{#} indicates the point of attachment of R² to the rest of the molecule,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   —N(H)C(═O)—(C₁-C₃-alkyl), —N(H)C(═O)—(C₃-C₄-cycloalkyl), —C(═O)OH,
   —C(═O)NH₂, and —S(═O)₂—(C₁-C₃-alkyl),
R⁴ and R⁵ represent, independently of each other, hydrogen or fluorine,
R⁶ represents a group selected from:
   hydroxy, C₁-C₄-alkoxy, (C₂-C₄-hydroxyalkyl)-O—, and
   (C₁-C₃-alkoxy)-(C₂-C₃-alkoxy)-,
R⁷ represents hydrogen, or a 2-hydroxyethyl group,
   or,
R² and R⁷ together represent a

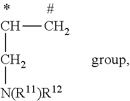

wherein * indicates the point of attachment of R² to the rest of the molecule,
and # indicates the point of attachment of R⁷ to the rest of the molecule,
R¹¹ and R¹² independently of each other represents hydrogen or a group selected from:
   C₂-C₃-haloalkyl, and —C(═O)H,
R¹⁴ represents, independently of each other, a group selected from:
   C₁-C₄-alkyl,
R¹⁵ represents hydrogen or a group selected from:
   —C(═O)R¹⁶
R¹⁶ represents a group selected from:

C₁-C₃-haloalkyl,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom selected from 0,
   said azetidinyl group being substituted with two halogen atoms,
   said 5- to 7-membered heterocycloalkyl group being optionally substituted, two times, identically or differently, with a substituent selected from:
      halogen atom,
         or an N-oxide, a salt, a tautomer or a stereoisomer of said compound, or a salt of said N-oxide, tautomer or stereoisomer.

A fifth aspect of the invention are compounds of formula (I) as defined herein, wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z represent CH,
R¹ represents hydrogen, or a group selected from:
   C₁-C₃-alkyl, trifluoromethyl, C₁-C₂-hydroxyalkyl, methoxymethyl-, —(CH₂)—S—R¹⁴,
   —(CH₂)—S(═O)₂—R¹⁴, —(CH₂)—N(R¹⁷)R¹⁸, 5-membered heteroaryl, and a 5- or 6-membered oxygen containing heterocycloalkyl group,
   in which 5-membered heteroaryl is connected to rest of the molecule via a carbon atom of the heteroaryl group,
   which 5-membered heteroaryl group is selected from:
   1,3-thiazolyl and pyrazolyl, which groups are optionally substituted, one or two times with a methyl group,
   which 5- or 6-membered oxygen containing heterocycloalkyl group is selected from:
   tetrahydrofuranyl and tetrahydro-2H-pyranyl,
R² represents hydrogen, fluorine, chlorine, or a group selected from:
   C₁-C₂-alkyl, cyclopropyl, —(CH₂)₃OH, methoxy-(C₂-C₃-alkyl)-, —(C₁-C₃-alkyl)-S—R¹⁴,
   —(C₁-C₃-alkyl)-S(═O)—R¹⁴, —(C₁-C₃-alkyl)-S(═O)₂—R¹⁴, —(CH₂)₂—S(═O)(═NR¹⁵)R¹⁴,
   —(CH₂)₃—S(═O)(═NR¹⁵)R¹⁴, —(C₁-C₄-alkyl)-N(R¹¹)R¹², —(C₁-C₄-alkyl)-N(R¹⁷)R¹⁸,
   (1,3-dioxolan-2-yl)-(CH₂)—, an azetidinyl group, azetidinyl-(CH₂)—, and a piperidinyl group,
   in which azetidinyl and piperidinyl are connected to rest of the molecule via a carbon atom of the azetidinyl ring or via a carbon atom of the piperidinyl ring,
   wherein azetidinyl is optionally substituted with a 2,2,2-trifluoroethyl group,
   wherein piperidinyl is optionally substituted with a substituent selected from:
      2,2,2-trifluoroethyl, 2,2-difluoroethyl, 3,3,3-trifluoropropyl and
      cyclopropylmethyl-,
   wherein at least one of R¹ and R² represents a group which connects via a carbon atom of said group to the rest of the molecule,
   or,
   R¹ and R² together represent a group, which is selected from:

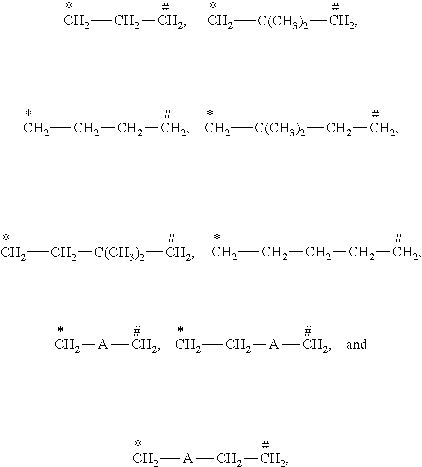

wherein A represents a group, which is selected from:
NH, N-ethyl, N-(2,2,2-trifluoroethyl), N-benzyl, O, S, and S(═O)₂,
and,
wherein * indicates the point of attachment of R¹ to the rest of the molecule,
and ^{#} indicates the point of attachment of R² to the rest of the molecule,
R³ represents fluorine, chlorine or a group selected from:
   hydroxy, methyl, cyclopropyl, difluoromethyl, trifluoromethyl, methoxy,
   trifluoromethoxy, —N(H)C(═O)—CH₃, —N(H)C(═O)-cyclopropyl, —C(═O)OH,
   —C(═O)NH₂, and —S(═O)₂—CH₃,
R⁴ and R⁵ represent, independently of each other, hydrogen or fluorine,
R⁶ represents a group selected from:
   hydroxy, C₁-C₂-alkoxy, (2-hydroxyethyl)-O—, and 2-methoxyethoxy-,
R⁷ represents hydrogen, or a 2-hydroxyethyl group,
   or,
R² and R⁷ together represent a

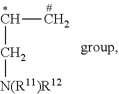

wherein * indicates the point of attachment of R² to the rest of the molecule,
and # indicates the point of attachment of R⁷ to the rest of the molecule,
R¹¹ and R¹² independently of each other represents hydrogen or a group selected from:
   2,2,2-trifluoroethyl, and C(═O)H,
R¹⁴ represents a methyl group,
R¹⁵ represents hydrogen or a group selected from:
   —C(═O)R¹⁶
R¹⁶ represents a trifluoromethyl group,
R¹⁷ and R¹⁸ together with the nitrogen to which they are attached form:
   an azetidinyl group or a 6-membered heterocycloalkyl group,
   said 6-membered heterocycloalkyl group optionally containing one additional heteroatom selected from 0,
   said azetidinyl group being substituted with two fluorine atoms,
   said 6-membered heterocycloalkyl group being optionally substituted with two fluorine atoms,
      or an N-oxide, a salt, a tautomer or a stereoisomer of said compound, or a salt of said N-oxide, tautomer or stereoisomer.

In accordance with a variant of the fifth aspect of the invention, the invention relates to compounds of formula (I) as defined herein, wherein
V, W, Y and Z independently of each other represent CH or CR³,
   or,
V represents N, and W, Y and Z independently of each other represent CH or CR³,
   or,
W represents N, and V, Y and Z independently of each other represent CH or CR³,
   or,
V and Y represent N, and W and Z represent CH,
R¹ represents hydrogen, or a group selected from:
   C₁-C₃ alkyl, C₁-C₂-hydroxyalkyl, methoxymethyl-, —(CH₂)—S—R¹⁴,
   —(CH₂)—S(═O)₂—R¹⁴, and —(CH₂)—N(R¹⁷)R¹⁸,
R² represents hydrogen, fluorine, chlorine, or a group selected from:
   C₁-C₂ alkyl, (CH₂)₃OH, methoxy-(C₂-C₃-alkyl)-, —(C₁-C₃-alkyl)-S—R¹⁴,
   —(C₁-C₃-alkyl)-S(═O)—R¹⁴, —(C₁-C₃-alkyl)-S(═O)₂—R¹⁴, —(CH₂)₃—S(═O)(═NR¹⁵)R¹⁴,
   —(C₁-C₄-alkyl)-N(R¹¹)R¹², —(C₁-C₄-alkyl)-N(R¹⁷)R¹⁸, (1,3-dioxolan-2-yl)-(CH₂)—, an
   azetidinyl group, azetidinyl-(CH₂)—, and a piperidinyl group,
      in which azetidinyl and piperidinyl are connected to rest of the molecule via a carbon atom of the azetidinyl ring or via a carbon atom of the piperidinyl ring,
      wherein azetidinyl is optionally substituted with a 2,2,2-trifluoroethyl group,
      wherein piperidinyl is optionally substituted with a 2,2,2-trifluoroethyl group,
      wherein at least one of R¹ and R² represents a group which connects via a carbon atom of said group to the rest of the molecule,
      or,
      R¹ and R² together represent a group, which is selected from:

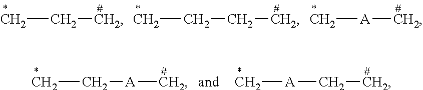

wherein A represents a group, which is selected from:
NH, N-ethyl, N-benzyl, O, S, and S(═O)₂,
and,
wherein * indicates the point of attachment of R¹ to the rest of the molecule,
and ^{#} indicates the point of attachment of R² to the rest of the molecule,
R³ represents fluorine, chlorine or a group selected from:
   hydroxy, methyl, cyclopropyl, difluoromethyl, trifluoromethyl, methoxy,
   —N(H)C(═O)—CH₃, —N(H)C(═O)-cyclopropyl, —C(═O)OH, —C(═O)NH₂, and —S(═O)₂—CH₃,
R⁴ and R⁵ represent, independently of each other, hydrogen or fluorine,
R⁶ represents a group selected from:
   hydroxy, C₁-C₂-alkoxy, (2-hydroxyethyl)-O—, and methoxy-ethoxy-,
R⁷ represents hydrogen, or a 2-hydroxyethyl group,
   or,
R² and R⁷ together represent a

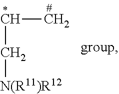

wherein * indicates the point of attachment of R² to the rest of the molecule,
and ^{#} indicates the point of attachment of R⁷ to the rest of the molecule,
R¹¹ and R¹² independently of each other represents hydrogen or a group selected from: 2,2,2-trifluoroethyl, and C(═O)H,
R¹⁴ represents a methyl group,
   R¹⁵ represents hydrogen or a group selected from:
   —C(═O)R¹⁶
R¹⁶ represents a trifluoromethyl group,
R¹⁷ and R¹⁸ together with the nitrogen to which they are attached form:
   an azetidinyl group or a 6-membered heterocycloalkyl group,
   said 6-membered heterocycloalkyl group optionally containing one additional heteroatom selected from O,
   said azetidinyl group being substituted with two fluorine atoms,
   said 6-membered heterocycloalkyl group being optionally substituted with two fluorine atoms,
      or an N-oxide, a salt, a tautomer or a stereoisomer of said compound, or a salt of said N-oxide, tautomer or stereoisomer.

In a further aspect of the invention compounds of formula (I) as described above are selected from the group consisting of:
2-[1-(4-ethoxy-2,6-difluorobenzyl)-1H-indazol-3-yl]-5-{2-[(R)-methylsulfinyl]ethyl}-N-(pyridin-4-yl)pyrimidin-4-amine,
2-[1-(4-ethoxy-2,6-difluorobenzyl)-1H-indazol-3-yl]-5-{3-[(R)-methylsulfinyl]propyl}-N-(pyridin-4-yl)pyrimidin-4-amine,
2-[1-(4-ethoxy-2,6-difluorobenzyl)-1H-indazol-3-yl]-5-[2-(methylsulfanyl)ethyl]-N-(pyridin-4-yl)pyrimidin-4-amine,
2-[1-(4-ethoxy-2,6-difluorobenzyl)-1H-indazol-3-yl]-5-[3-(methylsulfanyl)propyl]-N-(pyridin-4-yl)pyrimidin-4-amine,
2-[1-(4-ethoxy-2,6-difluorobenzyl)-1H-indazol-3-yl]-5-[(methylsulfanyl)methyl]-N-(pyridin-4-yl)pyrimidin-4-amine,
2-[1-(4-ethoxy-2,6-difluorobenzyl)-1H-indazol-3-yl]-5-methyl-N-(pyridin-4-yl)pyrimidin-4-amine,
N-({2-[1-(4-ethoxy-2,6-difluorobenzyl)-1H-indazol-3-yl]-4-(pyridin-4-ylamino)pyrimidin-5-yl}methyl)formamide,
2-[1-(4-ethoxy-2,6-difluorobenzyl)-1H-indazol-3-yl]-5-(2-methoxyethyl)-N-(pyridin-4-yl)pyrimidin-4-amine,
4-({2-[1-(4-ethoxy-2,6-difluorobenzyl)-1H-indazol-3-yl]-5-(3-methoxypropyl)pyrimidin-4-yl}amino)nicotinic acid,
2-[1-(4-ethoxy-2,6-difluorobenzyl)-1H-indazol-3-yl]-5-(3-methoxypropyl)-N-(pyridin-4-yl)pyrimidin-4-amine,
2-[1-(4-ethoxy-2,6-difluorobenzyl)-1H-indazol-3-yl]-5-[4-(morpholin-4-yl)butyl]-N-(pyridin-4-yl)pyrimidin-4-amine,
2-[1-(4-ethoxy-2,6-difluorobenzyl)-1H-indazol-3-yl]-5-[3-(morpholin-4-yl)propyl]-N-(pyridin-4-yl)pyrimidin-4-amine,
2-[1-(4-ethoxy-2,6-difluorobenzyl)-1H-indazol-3-yl]-5-[3-(morpholin-4-yl)propyl]-N-[3-(trifluoromethyl)pyridin-4-yl]pyrimidin-4-amine,
5-(1,3-dioxolan-2-ylmethyl)-2-[1-(4-ethoxy-2,6-difluorobenzyl)-1H-indazol-3-yl]-N-(pyridin-4-yl)pyrimidin-4-amine,
5-[2-(3,3-difluoropiperidin-1-yl)ethyl]-2-[1-(4-ethoxy-2,6-difluorobenzyl)-1H-indazol-3-yl]-N-(pyridin-4-yl)pyrimidin-4-amine,
5-[4-(3,3-difluoropiperidin-1-yl)butyl]-2-[1-(4-ethoxy-2,6-difluorobenzyl)-1H-indazol-3-yl]-N-(pyridin-4-yl)pyrimidin-4-amine,
5-[3-(4,4-difluoropiperidin-1-yl)propyl]-2-[1-(4-ethoxy-2,6-difluorobenzyl)-1H-indazol-3-yl]-N-(pyridin-4-yl)pyrimidin-4-amine,
5-[(3,3-difluoroazetidin-1-yl)methyl]-2-[1-(4-ethoxy-2,6-difluorobenzyl)-1H-indazol-3-yl]-N-(pyridin-4-yl)pyrimidin-4-amine,
5-[4-(3,3-difluoroazetidin-1-yl)butyl]-2-[1-(4-ethoxy-2,6-difluorobenzyl)-1H-indazol-3-yl]-N-(pyridin-4-yl)pyrimidin-4-amine,
2-[1-(4-ethoxy-2,6-difluorobenzyl)-1H-indazol-3-yl]-5-[2-(methylsulfonyl)ethyl]-N-(eparate-4-yl)pyrimidin-4-amine,
2-[1-(4-ethoxy-2,6-difluorobenzyl)-1H-indazol-3-yl]-5-[3-(methylsulfonyl)propyl]-N-(pyridin-4-yl)pyrimidin-4-amine,
2-[1-(4-ethoxy-2,6-difluorobenzyl)-1H-indazol-3-yl]-5-[(methylsulfonyl)methyl]-N-(pyridin-4-yl)pyrimidin-4-amine
2-[1-(4-ethoxy-2,6-difluorobenzyl)-1H-indazol-3-yl]-6-[(methylsulfonyl)methyl]-N-(pyridin-4-yl)pyrimidin-4-amine
2-{3,5-difluoro-4-[(3-{5-[2-(methylsulfonyl)ethyl]-4-(eparate-4-ylamino)pyrimidin-2-yl}-1H-indazol-1-yl)methyl]phenoxy}ethanol,
2-{3,5-difluoro-4-[(3-{5-[3-(methylsulfonyl)propyl]-4-(pyridin-4-ylamino)pyrimidin-2-yl}-1H-indazol-1-yl)methyl]phenoxy}ethanol,
2-[3,5-difluoro-4-({3-[5-{3-[(R)-methylsulfinyl]propyl}-4-(pyridin-4-ylamino)pyrimidin-2-yl]-1H-indazol-1-yl}methyl)phenoxy]ethanol,
2-[(2-{1-[2,6-difluoro-4-(2-hydroxyethoxy)benzyl]-1H-indazol-3-yl}-5-[(methyl-sulfonyl)methyl]pyrimidin-4-yl)(pyridin-4-yl)amino]ethanol,
2-{3,5-difluoro-4-[(3-{4-[(3-methoxypyridin-4-yl)amino]-6,7-dihydro-5H-cyclopenta[d]pyrimidin-2-yl}-1H-indazol-1-yl)methyl]phenoxy}ethanol,
N-{4-[(2-{1-[2,6-difluoro-4-(2-hydroxyethoxy)benzyl]-1H-indazol-3-yl}-6,7-dihydro-5H-cyclopenta[d]pyrimidin-4-yl)amino]pyridin-2-yl}acetamide,
2-{4-[(3-{4-[(2-aminopyridin-4-yl)amino]-6,7-dihydro-5H-cyclopenta[d]pyrimidin-2-yl}-1H-indazol-1-yl)methyl]-3,5-difluorophenoxy}ethanol,
2-{3,5-difluoro-4-[(3-{4-[(3-methylpyridin-4-yl)amino]-6,7-dihydro-5H-cyclopenta[d]pyrimidin-2-yl}-1H-indazol-1-yl)methyl]phenoxy}ethanol,
N-[4-({2-[1-(4-ethoxy-2,6-difluorobenzyl)-1H-indazol-3-yl]-6-[(1S)-1-hydroxyethyl]pyrimidin-4-yl}amino)pyridin-2-yl]acetamide,
2-[3,5-difluoro-4-({3-[4-(pyridazin-4-ylamino)-6,7-dihydro-5H-cyclopenta[d]pyrimidin-2-yl]-1H-indazol-1-yl}methyl)phenoxy]ethanol, salt with hydrochloric acid,
2-[3,5-difluoro-4-({3-[4-(pyridazin-4-ylamino)-6,7-dihydro-5H-cyclopenta[d]pyrimidin-2-yl]-1H-indazol-1-yl}methyl)phenoxy]ethanol,
2-{3,5-difluoro-4-[(3-{4-[(6-methylpyrimidin-4-yl)amino]-6,7-dihydro-5H-cyclopenta[d]pyrimidin-2-yl}-1H-indazol-1-yl)methyl]phenoxy}ethanol,
2-{3,5-difluoro-4-[(3-{4-[(2-methylpyridin-4-yl)amino]-6,7-dihydro-5H-cyclopenta[d]pyrimidin-2-yl}-1H-indazol-1-yl)methyl]phenoxy}ethanol,
3,5-difluoro-4-[(3-{5-[2-(methylsulfonyl)ethyl]-4-(pyridin-4-ylamino)pyrimidin-2-yl}-1H-indazol-1-yl)methyl]phenol
3,5-difluoro-4-[(3-{5-[3-(methylsulfonyl)propyl]-4-(pyridin-4-ylamino)pyrimidin-2-yl}-1H-indazol-1-yl)methyl]phenol,
3,5-difluoro-4-({3-[5-{3-[(R)-methylsulfinyl]propyl}-4-(pyridin-4-ylamino)pyrimidin-2-yl]-1H-indazol-1-yl}methyl)phenol,
{2-[1-(4-ethoxy-2,6-difluorobenzyl)-1H-indazol-3-yl]-6-[(3-methylpyridin-4-yl)-amino]pyrimidin-4-yl}methanol,
3,5-difluoro-4-[(3-{4-[(3-methylpyridin-4-yl)amino]-6,7-dihydro-5H-cyclopenta[d]-pyrimidin-2-yl}-1H-indazol-1-yl)methyl]phenol,
3,5-difluoro-4-[(3-{4-[(3-methoxypyridin-4-yl)amino]-6,7-dihydro-5H-cyclopenta[d]pyrimidin-2-yl}-1H-indazol-1-yl)methyl]phenol,
N-[4-({2-[1-(2,6-difluoro-4-hydroxybenzyl)-1H-indazol-3-yl]-6,7-dihydro-5H-cyclopenta[d]pyrimidin-4-yl}amino)pyridin-2-yl]acetamide,
3,5-difluoro-4-[(3-{4-[(2-methylpyridin-4-yl)amino]-6,7-dihydro-5H-cyclopenta[d]-pyrimidin-2-yl}-1H-indazol-1-yl)methyl]phenol,
3,5-difluoro-4-({3-[4-(pyridazin-4-ylamino)-6,7-dihydro-5H-cyclopenta[d]pyrimidin-2-yl]-1H-indazol-1-yl}methyl)phenol,
3,5-difluoro-4-[(3-{4-[(6-methylpyrimidin-4-yl)amino]-6,7-dihydro-5H-cyclopenta[d]pyrimidin-2-yl}-1H-indazol-1-yl)methyl]phenol,
3,5-difluoro-4-({3-[4-(pyrimidin-4-ylamino)-6,7-dihydro-5H-cyclopenta[d]pyrimidin-2-yl]-1H-indazol-1-yl}methyl)phenol,
3,5-difluoro-4-[(3-{4-[(2-methylpyrimidin-4-yl)amino]-6,7-dihydro-5H-cyclopenta[d]pyrimidin-2-yl}-1H-indazol-1-yl)methyl]phenol,
2-[1-(4-ethoxy-2,6-difluorobenzyl)-1H-indazol-3-yl]-5-[2-(S-methylsulfonimidoyl)ethyl]-N-(pyridin-4-yl)pyrimidin-4-amine,
N-[(2-{2-[1-(4-ethoxy-2,6-difluorobenzyl)-1H-indazol-3-yl]-4-(pyridin-4-ylamino)-pyrimidin-5-yl}ethyl)(methyl)oxido-λ6-sulfanylidene]-2,2,2-trifluoroacetamide,
5-(azetidin-3-yl)-2-[1-(4-ethoxy-2,6-difluorobenzyl)-1H-indazol-3-yl]-N-(pyridin-4-yl)pyrimidin-4-amine, salt with hydrochloric acid,
5-(azetidin-3-yl)-2-[1-(4-ethoxy-2,6-difluorobenzyl)-1H-indazol-3-yl]-N-(pyridin-4-yl)pyrimidin-4-amine,
5-(2-aminoethyl)-2-[1-(4-ethoxy-2,6-difluorobenzyl)-1H-indazol-3-yl]-N-(pyridin-4-yl)pyrimidin-4-amine, salt with hydrochloric acid,
5-(2-aminoethyl)-2-[1-(4-ethoxy-2,6-difluorobenzyl)-1H-indazol-3-yl]-N-(pyridin-4-yl)pyrimidin-4-amine,
2-[1-(4-ethoxy-2,6-difluorobenzyl)-1H-indazol-3-yl]-5-(piperidin-4-yl)-N-(pyridin-4-yl)pyrimidin-4-amine, salt with hydrochloric acid,
2-[1-(4-ethoxy-2,6-difluorobenzyl)-1H-indazol-3-yl]-5-(piperidin-4-yl)-N-(pyridin-4-yl)pyrimidin-4-amine,
5-(azetidin-3-ylmethyl)-2-[1-(4-ethoxy-2,6-difluorobenzyl)-1H-indazol-3-yl]-N-(pyridin-4-yl)pyrimidin-4-amine, salt with hydrochloric acid,
5-(azetidin-3-ylmethyl)-2-[1-(4-ethoxy-2,6-difluorobenzyl)-1H-indazol-3-yl]-N-(pyridin-4-yl)pyrimidin-4-amine,
5-(aminomethyl)-2-[1-(4-ethoxy-2,6-difluorobenzyl)-1H-indazol-3-yl]-N-(pyridin-4-yl)pyrimidin-4-amine, salt with hydrochloric acid,
5-(aminomethyl)-2-[1-(4-ethoxy-2,6-difluorobenzyl)-1H-indazol-3-yl]-N-(pyridin-4-yl)pyrimidin-4-amine,
5-(aminomethyl)-N-[2-(difluoromethyl)pyridin-4-yl]-2-[1-(4-ethoxy-2,6-difluorobenzyl)-1H-indazol-3-yl]pyrimidin-4-amine, salt with hydrochloric acid,
5-(aminomethyl)-N-[2-(difluoromethyl)pyridin-4-yl]-2-[1-(4-ethoxy-2,6-difluorobenzyl)-1H-indazol-3-yl]pyrimidin-4-amine,
N-[4-({5-(azetidin-3-ylmethyl)-2-[1-(4-ethoxy-2,6-difluorobenzyl)-1H-indazol-3-yl]pyrimidin-4-yl}amino)pyridin-2-yl]acetamide, salt with hydrochloric acid,
N-[4-({5-(azetidin-3-ylmethyl)-2-[1-(4-ethoxy-2,6-difluorobenzyl)-1H-indazol-3-yl]pyrimidin-4-yl}amino)pyridin-2-yl]acetamide,
5-(azetidin-3-ylmethyl)-2-[1-(4-ethoxy-2,6-difluorobenzyl)-1H-indazol-3-yl]-N-[2-trifluoromethyl)pyridin-4-yl]pyrimidin-4-amine, salt with hydrochloric acid,
5-(azetidin-3-ylmethyl)-2-[1-(4-ethoxy-2,6-difluorobenzyl)-1H-indazol-3-yl]-N-[2-trifluoromethyl)pyridin-4-yl]pyrimidin-4-amine,
5-(azetidin-3-ylmethyl)-N-[2-(difluoromethyl)pyridin-4-yl]-2-[1-(4-ethoxy-2,6-difluorobenzyl)-1H-indazol-3-yl]pyrimidin-4-amine, salt with hydrochloric acid,
5-(azetidin-3-ylmethyl)-N-[2-(difluoromethyl)pyridin-4-yl]-2-[1-(4-ethoxy-2,6-difluorobenzyl)-1H-indazol-3-yl]pyrimidin-4-amine,
5-(2-aminoethyl)-N-[2-(difluoromethyl)pyridin-4-yl]-2-[1-(4-ethoxy-2,6-difluorobenzyl)-1H-indazol-3-yl]pyrimidin-4-amine, salt with hydrochloric acid,
5-(2-aminoethyl)-N-[2-(difluoromethyl)pyridin-4-yl]-2-[1-(4-ethoxy-2,6-difluorobenzyl)-1H-indazol-3-yl]pyrimidin-4-amine,
5-(2-aminoethyl)-2-[1-(4-ethoxy-2,6-difluorobenzyl)-1H-indazol-3-yl]-N-[2-(tri-fluoromethyl)pyridin-4-yl]pyrimidin-4-amine, salt with hydrochloric acid,
5-(2-aminoethyl)-2-[1-(4-ethoxy-2,6-difluorobenzyl)-1H-indazol-3-yl]-N-[2-(tri-fluoromethyl)pyridin-4-yl]pyrimidin-4-amine,
5-(2-aminoethyl)-2-[1-(4-ethoxy-2,6-difluorobenzyl)-1H-indazol-3-yl]-N-(2-methylpyridin-4-yl)pyrimidin-4-amine, salt with hydrochloric acid,
5-(2-aminoethyl)-2-[1-(4-ethoxy-2,6-difluorobenzyl)-1H-indazol-3-yl]-N-(2-methylpyridin-4-yl)pyrimidin-4-amine,
N-[4-({5-(2-aminoethyl)-2-[1-(4-ethoxy-2,6-difluorobenzyl)-1H-indazol-3-yl]pyrimidin-4-yl}amino)pyridin-2-yl]acetamide, salt with hydrochloric acid,
N-[4-({5-(2-aminoethyl)-2-[1-(4-ethoxy-2,6-difluorobenzyl)-1H-indazol-3-yl]pyrimidin-4-yl}amino)pyridin-2-yl]acetamide,
2-[1-(4-ethoxy-2,6-difluorobenzyl)-1H-indazol-3-yl]-N-(pyridin-4-yl)-6,7-dihydro-5H-pyrrolo[3,4-d]pyrimidin-4-amine, salt with hydrochloric acid,
2-[1-(4-ethoxy-2,6-difluorobenzyl)-1H-indazol-3-yl]-N-(pyridin-4-yl)-6,7-dihydro-5H-pyrrolo[3,4-d]pyrimidin-4-amine,
2-[1-(4-ethoxy-2,6-difluorobenzyl)-1H-indazol-3-yl]-N-[2-(trifluoromethyl)pyridin-4-yl]-6,7-dihydro-5H-pyrrolo[3,4-d]pyrimidin-4-amine, salt with hydrochloric acid,
2-[1-(4-ethoxy-2,6-difluorobenzyl)-1H-indazol-3-yl]-N-[2-(trifluoromethyl)pyridin-4-yl]-6,7-dihydro-5H-pyrrolo[3,4-d]pyrimidin-4-amine,
2-[1-(4-ethoxy-2,6-difluorobenzyl)-1H-indazol-3-yl]-N-(pyridin-4-yl)-5,6,7,8-tetrahydropyrido[4,3-d]pyrimidin-4-amine,
2-[1-(4-ethoxy-2,6-difluorobenzyl)-1H-indazol-3-yl]-N-[2-(trifluoromethyl)pyridin-4-yl]-5,6,7,8-tetrahydropyrido[4,3-d]pyrimidin-4-amine,
5-(2-amino-2-methylpropyl)-2-[1-(4-ethoxy-2,6-difluorobenzyl)-1H-indazol-3-yl]-N-(pyridin-4-yl)pyrimidin-4-amine, salt with hydrochloric acid,
5-(2-amino-2-methylpropyl)-2-[1-(4-ethoxy-2,6-difluorobenzyl)-1H-indazol-3-yl]-N-(pyridin-4-yl)pyrimidin-4-amine,
4-({2-[1-(4-ethoxy-2,6-difluorobenzyl)-1H-indazol-3-yl]-5-(3-methoxypropyl)pyrimidin-4-yl}amino)nicotinamide,
4-({2-[1-(4-ethoxy-2,6-difluorobenzyl)-1H-indazol-3-yl]-6-methylpyrimidin-4-yl}-amino)pyridine-3-carboxamide,
2-[1-(4-ethoxy-2,6-difluorobenzyl)-1H-indazol-3-yl]-N-(pyridin-4-yl)-5-{[1-(2,2,2-trifluoroethyl)azetidin-3-yl]methyl}pyrimidin-4-amine,
2-[1-(4-ethoxy-2,6-difluorobenzyl)-1H-indazol-3-yl]-N-(pyridin-4-yl)-5-[1-(2,2,2-trifluoroethyl)azetidin-3-yl]pyrimidin-4-amine,
N-({2-[1-(4-ethoxy-2,6-difluorobenzyl)-1H-indazol-3-yl]-7-(pyridin-4-yl)-6,7-dihydro-5H-pyrrolo[2,3d]pyrimidin-5-yl}methyl)-2,2,2-trifluoroethanamine,
N-{4-[(2-[1-(4-ethoxy-2,6-difluorobenzyl)-1H-indazol-3-yl]-5-{[1-(2,2,2-trifluoro-ethyl)azetidin-3-yl]methyl}pyrimidin-4-yl)amino]pyridin-2-yl}acetamide,
2-[1-(4-ethoxy-2,6-difluorobenzyl)-1H-indazol-3-yl]-6-methyl-N-(pyridin-4-yl)pyrimidin-4-amine,
2-[1-(4-ethoxy-2,6-difluorobenzyl)-1H-indazol-3-yl]-N-(pyridin-4-yl)-6,7-dihydro-5H-cyclopenta[d]pyrimidin-4-amine,
2-[1-(4-ethoxy-2,6-difluorobenzyl)-1H-indazol-3-yl]-6-(propan-2-yl)-N-(pyridin-4-yl)pyrimidin-4-amine,
2-[1-(4-ethoxy-2,6-difluorobenzyl)-1H-indazol-3-yl]-6-(methoxymethyl)-N-(pyridin-4-yl)pyrimidin-4-amine,
2-[1-(4-ethoxy-2,6-difluorobenzyl)-1H-indazol-3-yl]-5-fluoro-6-methyl-N-(pyridin-4-yl)pyrimidin-4-amine,
2-[1-(4-ethoxy-2,6-difluorobenzyl)-1H-indazol-3-yl]-N-(2-methylpyridin-4-yl)-6,7-dihydro-5H-cyclopenta[d]pyrimidin-4-amine,
2-[1-(4-ethoxy-2,6-difluorobenzyl)-1H-indazol-3-yl]-5-ethyl-6-methyl-N-(pyridin-4-yl)pyrimidin-4-amine,
2-[1-(4-ethoxy-2,6-difluorobenzyl)-1H-indazol-3-yl]-6-ethyl-N-(pyridin-4-yl)pyrimidin-4-amine,
2-[1-(4-ethoxy-2,6-difluorobenzyl)-1H-indazol-3-yl]-6-(morpholin-4-ylmethyl)-N-(pyridin-4-yl)pyrimidin-4-amine,
2-[1-(4-ethoxy-2,6-difluorobenzyl)-1H-indazol-3-yl]-N-(3-methylpyridin-4-yl)-6-(morpholin-4-ylmethyl)pyrimidin-4-amine,
N-[4-({2-[1-(4-ethoxy-2,6-difluorobenzyl)-1H-indazol-3-yl]-6-(morpholin-4-yl-methyl)pyrimidin-4-yl}amino)pyridin-2-yl]acetamide,
7-benzyl-2-[1-(4-ethoxy-2,6-difluorobenzyl)-1H-indazol-3-yl]-N-(pyridin-4-yl)-5,6,7,8-tetrahydropyrido[3,4-d]pyrimidin-4-amine,
7-benzyl-2-[1-(4-ethoxy-2,6-difluorobenzyl)-1H-indazol-3-yl]-N-(3-methylpyridin-4-yl)-5,6,7,8-tetrahydropyrido[3,4-d]pyrimidin-4-amine,
2-[1-(4-ethoxy-2,6-difluorobenzyl)-1H-indazol-3-yl]-6-[(methylsulfanyl)methyl]-N-(pyridin-4-yl)pyrimidin-4-amine,
N-[4-({2-[1-(4-ethoxy-2,6-difluorobenzyl)-1H-indazol-3-yl]-6,7-dihydro-5H-cyclopenta[d]pyrimidin-4-yl}amino)pyridin-2-yl]acetamide,
2-[1-(4-ethoxy-2,6-difluorobenzyl)-1H-indazol-3-yl]-6-(methoxymethyl)-N-(3-methylpyridin-4-yl)pyrimidin-4-amine,
N-[4-({2-[1-(4-ethoxy-2,6-difluorobenzyl)-1H-indazol-3-yl]-6-(methoxymethyl)pyrimidin-4-yl}amino)pyridin-2-yl]acetamide,
2-[1-(4-ethoxy-2,6-difluorobenzyl)-1H-indazol-3-yl]-5-fluoro-6-methyl-N-[2-(tri-fluoromethyl)pyridin-4-yl]pyrimidin-4-amine,
N-[4-({2-[1-(4-ethoxy-2,6-difluorobenzyl)-1H-indazol-3-yl]-6-ethylpyrimidin-4-yl}-amino)pyridin-2-yl]acetamide,
5-chloro-2-[1-(4-ethoxy-2,6-difluorobenzyl)-1H-indazol-3-yl]-6-methyl-N-(pyridin-4-yl)pyrimidin-4-amine,
5-fluoro-2-[1-(4-methoxybenzyl)-1H-indazol-3-yl]-6-methyl-N-(pyridin-4-yl)pyrimidin-4-amine,
2-[1-(4-ethoxy-2,6-difluorobenzyl)-1H-indazol-3-yl]-N-(6-methoxypyrimidin-4-yl)-6,7-dihydro-5H-cyclopenta[d]pyrimidin-4-amine,
N-(2,6-dimethylpyrimidin-4-yl)-2-[1-(4-ethoxy-2,6-difluorobenzyl)-1H-indazol-3-yl]-6,7-dihydro-5H-cyclopenta[d]pyrimidin-4-amine,
N-[4-({2-[1-(4-ethoxy-2,6-difluorobenzyl)-1H-indazol-3-yl]-6,7-dihydro-5H-cyclopenta[d]pyrimidin-4-yl}amino)pyridin-2-yl]cyclopropanecarboxamide,
N-(3-chloropyridin-4-yl)-2-{(1-[2,6-difluoro-4-(2-methoxyethoxy)benzyl]-1H-indazol-3-yl}-6,7-dihydro-5H-cyclopenta[d]pyrimidin-4-amine,
2-{1-[2,6-difluoro-4-(2-methoxyethoxy)benzyl]-1H-indazol-3-yl}-N-(3-methoxypyridin-4-yl)-6,7-dihydro-5H-cyclopenta[d]pyrimidin-4-amine,
N-(6-chloropyrimidin-4-yl)-2-[1-(4-ethoxy-2,6-difluorobenzyl)-1H-indazol-3-yl]-6,7-dihydro-5H-cyclopenta[d]pyrimidin-4-amine,
2-[1-(4-ethoxy-2,6-difluorobenzyl)-1H-indazol-3-yl]-N-[2-(methylsulfonyl)pyridin-4-yl]-6-(morpholin-4-ylmethyl)pyrimidin-4-amine,
N-(3-chloropyridin-4-yl)-2-[1-(4-ethoxy-2,6-difluorobenzyl)-1H-indazol-3-yl]-6-(morpholin-4-ylmethyl)pyrimidin-4-amine,
N-{2-[1-(4-ethoxy-2,6-difluorobenzyl)-1H-indazol-3-yl]-6-(morpholin-4-ylmethyl)-pyrimidin-4-yl}pyridazin-4-amine,
2-{1-[2,6-difluoro-4-(2-methoxyethoxy)benzyl]-1H-indazol-3-yl}-N-[2-(difluoro-methyl)pyridin-4-yl]-6-(morpholin-4-ylmethyl)pyrimidin-4-amine
2-{1-[2,6-difluoro-4-(2-methoxyethoxy)benzyl]-1H-indazol-3-yl}-6-(morpholin-4-yl-methyl)-N-(pyrimidin-4-yl)pyrimidin-4-amine
2-[1-(4-ethoxy-2,6-difluorobenzyl)-1H-indazol-3-yl]-N-(5-methylpyrimidin-4-yl)-6-(morpholin-4-ylmethyl)pyrimidin-4-amine,
N-[2-{1-[2,6-difluoro-4-(2-methoxyethoxy)benzyl]-1H-indazol-3-yl}-6-(morpholin-4-ylmethyl)pyrimidin-4-yl]pyridazin-4-amine,
N-(3-chloropyridin-4-yl)-2-{1-[2,6-difluoro-4-(2-methoxyethoxy)benzyl]-1H-indazol-3-yl}-6-(morpholin-4-ylmethyl)pyrimidin-4-amine,
2-{1-[2,6-difluoro-4-(2-methoxyethoxy)benzyl]-1H-indazol-3-yl}-N-(3-methylpyridin-4-yl)-6-(morpholin-4-ylmethyl)pyrimidin-4-amine,
2-{1-[2,6-difluoro-4-(2-methoxyethoxy)benzyl]-1H-indazol-3-yl}-N-(3-methoxypyridin-4-yl)-6-(morpholin-4-ylmethyl)pyrimidin-4-amine,
2-{1-[2,6-difluoro-4-(2-methoxyethoxy)benzyl]-1H-indazol-3-yl}-6-(morpholin-4-ylmethyl)-N-(pyridin-4-yl)pyrimidin-4-amine,
N-(2,6-dimethylpyrimidin-4-yl)-2-[1-(4-ethoxy-2,6-difluorobenzyl)-1H-indazol-3-yl]-6-(morpholin-4-ylmethyl)pyrimidin-4-amine,
6-({2-[1-(4-ethoxy-2,6-difluorobenzyl)-1H-indazol-3-yl]-6-(morpholin-4-ylmethyl)-pyrimidin-4-yl}amino)pyrimidin-4-ol,
4-({2-[1-(4-ethoxy-2,6-difluorobenzyl)-1H-indazol-3-yl]-6-(morpholin-4-ylmethyl)-pyrimidin-4-yl}amino)pyridin-2-ol,
2-[1-(4-ethoxy-2,6-difluorobenzyl)-1H-indazol-3-yl]-6-(morpholin-4-ylmethyl)-N-(pyrimidin-4-yl)pyrimidin-4-amine,
2-[1-(4-ethoxy-2,6-difluorobenzyl)-1H-indazol-3-yl]-6-(methoxymethyl)-N-(2-methylpyridin-4-yl)pyrimidin-4-amine,
2-{3,5-difluoro-4-[(3-{4-(methoxymethyl)-6-[(3-methoxypyridin-4-yl)amino]pyrimidin-2-yl}-1H-indazol-1-yl)methyl]phenoxy}ethanol,
2-{3,5-difluoro-4-[(3-{4-(methoxymethyl)-6-[(3-methylpyridin-4-yl)amino]pyrimidin-2-yl}-1H-indazol-1-yl)methyl]phenoxy}ethanol,
2-{3,5-difluoro-4-[(3-{4-(methoxymethyl)-6-[(2-methylpyridin-4-yl)amino]pyrimidin-2-yl}-1H-indazol-1-yl)methyl]phenoxy}ethanol
2-[1-(4-ethoxy-2,6-difluorobenzyl)-1H-indazol-3-yl]-N-(1,3,5-triazin-2-yl)-6,7-dihydro-5H-cyclopenta[d]pyrimidin-4-amine,
N-(6-chloropyridazin-4-yl)-2-[1-(4-ethoxy-2,6-difluorobenzyl)-1H-indazol-3-yl]-6,7-dihydro-5H-cyclopenta[d]pyrimidin-4-amine,
2-[1-(4-ethoxy-2,6-difluorobenzyl)-1H-indazol-3-yl]-N-(2-methylpyrimidin-4-yl)-6,7-dihydro-5H-cyclopenta[d]pyrimidin-4-amine,
3-{2-[1-(4-ethoxy-2,6-difluorobenzyl)-1H-indazol-3-yl]-4-(pyridin-4-ylamino)pyrimidin-5-yl}propan-1-ol,
2-[1-(4-ethoxy-2,6-difluorobenzyl)-1H-indazol-3-yl]-N-(3-fluoropyridin-4-yl)-6,7-dihydro-5H-cyclopenta[d]pyrimidin-4-amine,
2-[1-(4-ethoxy-2,6-difluorobenzyl)-1H-indazol-3-yl]-N-(3-methylpyridin-4-yl)-6,7-dihydro-5H-cyclopenta[d]pyrimidin-4-amine,
2-[1-(4-ethoxy-2,6-difluorobenzyl)-1H-indazol-3-yl]-N-[3-(trifluoromethyl)pyridin-4-yl]-6,7-dihydro-5H-cyclopenta[d]pyrimidin-4-amine,
2-[1-(4-ethoxy-2,6-difluorobenzyl)-1H-indazol-3-yl]-N-(3-methoxypyridin-4-yl)-6,7-dihydro-5H-cyclopenta[d]pyrimidin-4-amine,
2-[1-(4-ethoxy-2,6-difluorobenzyl)-1H-indazol-3-yl]-N-(pyridazin-4-yl)-6,7-dihydro-5H-cyclopenta[d]pyrimidin-4-amine,
2-[1-(4-ethoxy-2,6-difluorobenzyl)-1H-indazol-3-yl]-N-(pyrimidin-4-yl)-6,7-dihydro-5H-cyclopenta[d]pyrimidin-4-amine,
N-(3-chloropyridin-4-yl)-2-[1-(4-ethoxy-2,6-difluorobenzyl)-1H-indazol-3-yl]-6,7-dihydro-5H-cyclopenta[d]pyrimidin-4-amine,
N-(3-cyclopropylpyridin-4-yl)-2-[1-(4-ethoxy-2,6-difluorobenzyl)-1H-indazol-3-yl]-6,7-dihydro-5H-cyclopenta[d]pyrimidin-4-amine,
4-({2-[1-(4-ethoxy-2,6-difluorobenzyl)-1H-indazol-3-yl]-6,7-dihydro-5H-cyclopenta[d]pyrimidin-4-yl}amino)pyridine-3-carboxylic acid,
2-[1-(4-ethoxy-2,6-difluorobenzyl)-1H-indazol-3-yl]-N-(pyridin-4-yl)-5,7-dihydrofuro[3,4-d]pyrimidin-4-amine,
2-[1-(4-ethoxy-2,6-difluorobenzyl)-1H-indazol-3-yl]-6-ethyl-N-(pyridin-4-yl)-5,6,7,8-tetrahydropyrido[4,3-d]pyrimidin-4-amine,
2-[1-(4-ethoxy-2,6-difluorobenzyl)-1H-indazol-3-yl]-6-ethyl-N-(2-methylpyridin-4-yl)-5,6,7,8-tetrahydropyrido[4,3-d]pyrimidin-4-amine,
2-[1-(4-ethoxy-2,6-difluorobenzyl)-1H-indazol-3-yl]-6-(piperidin-1-ylmethyl)-N-(pyridin-4-yl)pyrimidin-4-amine
2-[1-(4-ethoxy-2,6-difluorobenzyl)-1H-indazol-3-yl]-N-(pyridin-4-yl)-5-{2-[(2,2,2-trifluoroethyl)amino]ethyl}pyrimidin-4-amine,
2-[1-(4-ethoxy-2,6-difluorobenzyl)-1H-indazol-3-yl]-5-(piperidin-4-yl)-N-(pyridin-4-yl)pyrimidin-4-amine,
N-[4-({2-[1-(4-ethoxy-2,6-difluorobenzyl)-1H-indazol-3-yl]-5-(piperidin-4-yl)pyrimidin-4-yl}amino)pyridin-2-yl]acetamide,
2-[1-(4-ethoxy-2,6-difluorobenzyl)-1H-indazol-3-yl]-N-(2-methylpyrimidin-4-yl)-5-(piperidin-4-yl)pyrimidin-4-amine,
2-[1-(4-ethoxy-2,6-difluorobenzyl)-1H-indazol-3-yl]-5-(piperidin-4-yl)-N-(pyrimidin-4-yl)pyrimidin-4-amine,
2-[1-(4-ethoxy-2,6-difluorobenzyl)-1H-indazol-3-yl]-N-(pyridin-4-yl)-5,6,7,8-tetrahydropyrido[3,4-d]pyrimidin-4-amine,
2-[1-(4-ethoxy-2,6-difluorobenzyl)-1H-indazol-3-yl]-N-(pyridin-4-yl)-5,7-dihydrothieno[3,4-d]pyrimidin-4-amine,
2-[1-(4-ethoxy-2,6-difluorobenzyl)-1H-indazol-3-yl]-N-(3-methylpyridin-4-yl)-5,7-dihydrothieno[3,4-d]pyrimidin-4-amine,
N-[4-({2-[1-(4-ethoxy-2,6-difluorobenzyl)-1H-indazol-3-yl]-5,7-dihydrothieno[3,4-d]pyrimidin-4-yl}amino)pyridin-2-yl]acetamide
2-[1-(4-ethoxy-2,6-difluorobenzyl)-1H-indazol-3-yl]-N-(2-methylpyridin-4-yl)-5,7-dihydrothieno[3,4-d]pyrimidin-4-amine,
2-[1-(4-ethoxy-2,6-difluorobenzyl)-1H-indazol-3-yl]-N-(2-methylpyridin-4-yl)-5,7-dihydrothieno[3,4-d]pyrimidin-4-amine 6,6-dioxide,
2-[1-(4-ethoxy-2,6-difluorobenzyl)-1H-indazol-3-yl]-N-[2-(methylsulfonyl)pyridin-4-yl]-6,7-dihydro-5H-cyclopenta[d]pyrimidin-4-amine,
2-[1-(4-ethoxy-2,6-difluorobenzyl)-1H-indazol-3-yl]-N-(2-methoxypyridin-4-yl)-6,7-dihydro-5H-cyclopenta[d]pyrimidin-4-amine,
N-[2-(difluoromethyl)pyridin-4-yl]-2-[1-(4-ethoxy-2,6-difluorobenzyl)-1H-indazol-3-yl]-6,7-dihydro-5H-cyclopenta[d]pyrimidin-4-amine,
2-[1-(4-ethoxy-2,6-difluorobenzyl)-1H-indazol-3-yl]-N-(2-methylpyridin-4-yl)-6-(morpholin-4-ylmethyl)pyrimidin-4-amine,
2-[1-(4-ethoxy-2,6-difluorobenzyl)-1H-indazol-3-yl]-N-(2-methoxypyridin-4-yl)-6-(morpholin-4-ylmethyl)pyrimidin-4-amine,
N-[4-({2-[1-(4-ethoxy-2,6-difluorobenzyl)-1H-indazol-3-yl]-6-(morpholin-4-yl-methyl)pyrimidin-4-yl}amino)pyridin-2-yl]cyclopropanecarboxamide
2-[1-(4-ethoxy-2,6-difluorobenzyl)-1H-indazol-3-yl]-N-(3-fluoropyridin-4-yl)-6-(morpholin-4-ylmethyl)pyrimidin-4-amine,
2-[1-(4-ethoxy-2,6-difluorobenzyl)-1H-indazol-3-yl]-N-(3-methoxypyridin-4-yl)-6-(morpholin-4-ylmethyl)pyrimidin-4-amine,
N-[2-(difluoromethyl)pyridin-4-yl]-2-[1-(4-ethoxy-2,6-difluorobenzyl)-1H-indazol-3-yl]-5,7-dihydrothieno[3,4-d]pyrimidin-4-amine 6,6-dioxide,
2-[1-(4-ethoxy-2,6-difluorobenzyl)-1H-indazol-3-yl]-N-(2-methylpyridin-4-yl)-5,7-dihydrofuro[3,4-d]pyrimidin-4-amine,
2-[1-(4-ethoxy-2,6-difluorobenzyl)-1H-indazol-3-yl]-N-(3-methoxypyridin-4-yl)-5,7-dihydrofuro[3,4-d]pyrimidin-4-amine,
2-[1-(4-ethoxy-2,6-difluorobenzyl)-1H-indazol-3-yl]-N-(3-methylpyridin-4-yl)-5,7-dihydrofuro[3,4-d]pyrimidin-4-amine,
N-[2-(difluoromethyl)pyridin-4-yl]-2-[1-(4-ethoxy-2,6-difluorobenzyl)-1H-indazol-3-yl]-5,6,7,8-tetrahydroquinazolin-4-amine,
2-[1-(4-ethoxy-2,6-difluorobenzyl)-1H-indazol-3-yl]-N-(3-methylpyridin-4-yl)-5,6,7,8-tetrahydroquinazolin-4-amine,
2-[1-(4-ethoxy-2,6-difluorobenzyl)-1H-indazol-3-yl]-N-(pyridin-4-yl)-5,6,7,8-tetrahydroquinazolin-4-amine,
N-[4-({2-[1-(4-ethoxy-2,6-difluorobenzyl)-1H-indazol-3-yl]-5-[1-(2,2,2-trifluoro-ethyl)piperidin-4-yl]pyrimidin-4-yl}amino)pyridin-2-yl]acetamide,
2-[1-(4-ethoxy-2,6-difluorobenzyl)-1H-indazol-3-yl]-N-(2-methylpyrimidin-4-yl)-5-[1-(2,2,2-trifluoroethyl)piperidin-4-yl]pyrimidin-4-amine,
2-[1-(4-ethoxy-2,6-difluorobenzyl)-1H-indazol-3-yl]-N-(pyrimidin-4-yl)-5-[1-(2,2,2-trifluoroethyl)piperidin-4-yl]pyrimidin-4-amine,
2-[1-(4-ethoxy-2,6-difluorobenzyl)-1H-indazol-3-yl]-N-(6-methylpyrimidin-4-yl)-6,7-dihydro-5H-cyclopenta[d]pyrimidin-4-amine,
2-{1-[2,6-difluoro-4-(2-methoxyethoxy)benzyl]-1H-indazol-3-yl}-N-(3-methoxypyridin-4-yl)-5-methylpyrimidin-4-amine,
5-cyclopropyl-2-{1-[2,6-difluoro-4-(2-methoxyethoxy)benzyl]-1H-indazol-3-yl}-N-(3-methoxypyridin-4-yl)pyrimidin-4-amine,
N-{4-[(2-{1-[2,6-difluoro-4-(2-hydroxyethoxy)benzyl]-1H-indazol-3-yl}-5-[1-(2,2,2-tri-fluoroethyl)piperidin-4-yl]pyrimidin-4-yl)amino]pyridin-2-yl}acetamide,
2-{4-[(3-{4-[(3-chloropyridin-4-yl)amino]-5-[1-(2,2,2-trifluoroethyl)piperidin-4-yl]-pyrimidin-2-yl}-1H-indazol-1-yl)methyl]-3,5-difluorophenoxy}ethanol,
2-{4-[(3-{4-[(3-chloropyridin-4-yl)amino]-5-[1-(3,3,3-trifluoropropyl)piperidin-4-yl]-pyrimidin-2-yl}-1H-indazol-1-yl)methyl]-3,5-difluorophenoxy}ethanol,
2-{3,5-difluoro-4-[(3-{4-[(2-methylpyrimidin-4-yl)amino]-5-[1-(2,2,2-trifluoroethyl)-piperidin-4-yl]pyrimidin-2-yl}-1H-indazol-1-yl)methyl]phenoxy}ethanol,
2-{4-[(3-{5-[1-(2,2-difluoroethyl)piperidin-4-yl]-4-[(2-methylpyrimidin-4-yl)amino]-pyrimidin-2-yl}-1H-indazol-1-yl)methyl]-3,5-difluorophenoxy}ethanol,
2-{3,5-difluoro-4-[(3-{4-[(2-methylpyrimidin-4-yl)amino]-6,7-dihydro-5H-cyclopenta[d]-pyrimidin-2-yl}-1H-indazol-1-yl)methyl]phenoxy}ethanol,
2-[3,5-difluoro-4-({3-[4-(pyrimidin-4-ylamino)-6,7-dihydro-5H-cyclopenta[d]pyrimidin-2-yl]-1H-indazol-1-yl}methyl)phenoxy]ethanol,
2-{4-[(3-{4-[(2,5-dimethylpyridin-4-yl)amino]-6,7-dihydro-5H-cyclopenta[d]pyrimidin-2-yl}-1H-indazol-1-yl)methyl]-3,5-difluorophenoxy}ethanol,
2-{3,5-difluoro-4-[(3-{4-[(5-fluoro-2-methylpyridin-4-yl)amino]-6,7-dihydro-5H-cyclopenta[d]pyrimidin-2-yl}-1H-indazol-1-yl)methyl]phenoxy}ethanol,
2-{4-[(3-{6,6-dimethyl-4-[(2-methylpyrimidin-4-yl)amino]-6,7-dihydro-5H-cyclopenta[d]-pyrimidin-2-yl}-1H-indazol-1-yl)methyl]-3,5-difluorophenoxy}ethanol,
2-{4-[(3-{5-[1-(cyclopropylmethyl)piperidin-4-yl]-4-[(2-methylpyrimidin-4-yl)amino]-pyrimidin-2-yl}-1H-indazol-1-yl)methyl]-3,5-difluorophenoxy}ethanol,
2-{4-[(3-{7,7-dimethyl-4-[(2-methylpyrimidin-4-yl)amino]-5,6,7,8-tetrahydroquinazolin-2-yl}-1H-indazol-1-yl)methyl]-3,5-difluorophenoxy}ethanol,
2-{4-[(3-{4-[(3-chloropyridin-4-yl)amino]-7,7-dimethyl-5,6,7,8-tetrahydroquinazolin-2-yl}-1H-indazol-1-yl)methyl]-3,5-difluorophenoxy}ethanol,
2-{3,5-difluoro-4-[(3-{4-[(3-methoxypyridin-4-yl)amino]-7,7-dimethyl-5,6,7,8-tetrahydroquinazolin-2-yl}-1H-indazol-1-yl)methyl]phenoxy}ethanol,
2-{3,5-difluoro-4-[(3-{4-methyl-6-[(2-methylpyrimidin-4-yl)amino]pyrimidin-2-yl}-1H-indazol-1-yl)methyl]phenoxy}ethanol,
2-{3,5-difluoro-4-[(3-{4-isopropyl-6-[(2-methylpyrimidin-4-yl)amino]pyrimidin-2-yl}-1H-indazol-1-yl)methyl]phenoxy}ethanol,
2-{3,5-difluoro-4-[(3-{4-isopropyl-6-[(3-methoxypyridin-4-yl)amino]pyrimidin-2-yl}-1H-indazol-1-yl)methyl]phenoxy}ethanol,
2-{4-[(3-{4-[(2,5-dimethylpyridin-4-yl)amino]-6-isopropylpyrimidin-2-yl}-1H-indazol-1-yl)methyl]-3,5-difluorophenoxy}ethanol,
2-{3,5-difluoro-4-[(3-{4-[(3-methoxypyridin-4-yl)amino]-5,6-dimethylpyrimidin-2-yl}-1H-indazol-1-yl)methyl]phenoxy}ethanol,
2-{3,5-difluoro-4-[(3-{5-fluoro-4-methyl-6-[(2-methylpyrimidin-4-yl)amino]pyrimidin-2-yl}-1H-indazol-1-yl)methyl]phenoxy}ethanol,
2-{3,5-difluoro-4-[(3-{4-[(3-methoxypyridin-4-yl)amino]-6-methylpyrimidin-2-yl}-1H-indazol-1-yl)methyl]phenoxy}ethanol,
2-{3,5-difluoro-4-[(3-{4-[(3-methoxypyridin-4-yl)amino]-5-methylpyrimidin-2-yl}-1H-indazol-1-yl)methyl]phenoxy}ethanol,
2-(3,5-difluoro-4-{[3-(4-methyl-6-{[3-(trifluoromethoxy)pyridin-4-yl]amino}pyrimidin-2-yl)-1H-indazol-1-yl]methyl}phenoxy)ethanol,
2-{4-[(3-{5-cyclopropyl-4-[(3-methoxypyridin-4-yl)amino]pyrimidin-2-yl}-1H-indazol-1-yl)methyl]-3,5-difluorophenoxy}ethanol,
2-[3,5-difluoro-4-({3-[4-(morpholin-4-ylmethyl)-6-(pyridazin-4-ylamino)pyrimidin-2-yl]-1H-indazol-1-yl}methyl)phenoxy]ethanol,
2-[3,5-difluoro-4-({3-[4-(morpholin-4-ylmethyl)-6-(pyridin-4-ylamino)pyrimidin-2-yl]-1H-indazol-1-yl}methyl)phenoxy]ethanol,
2-{3,5-difluoro-4-[(3-{4-[(2-methylpyrimidin-4-yl)amino]-6-(2,2,2-trifluoroethyl)-6,7-dihydro-5H-pyrrolo[3,4-d]pyrimidin-2-yl}-1H-indazol-1-yl)methyl]phenoxy}ethanol,
2-[3,5-difluoro-4-({3-[4-(pyrimidin-4-ylamino)-6-(2,2,2-trifluoroethyl)-6,7-dihydro-5H-pyrrolo[3,4-d]pyrimidin-2-yl]-1H-indazol-1-yl}methyl)phenoxy]ethanol,
2-{3,5-difluoro-4-[(3-{4-[(3-methoxypyridin-4-yl)amino]-6-(2,2,2-trifluoroethyl)-6,7-dihydro-5H-pyrrolo[3,4-d]pyrimidin-2-yl}-1H-indazol-1-yl)methyl]phenoxy}ethanol,
N-{4-[(2-{1-[2,6-difluoro-4-(2-hydroxyethoxy)benzyl]-1H-indazol-3-yl}-7,7-dimethyl-5,6,7,8-tetrahydroquinazolin-4-yl)amino]pyridin-2-yl}acetamide,
2-{4-[(3-{4-[(2,5-dimethylpyridin-4-yl)amino]-7,7-dimethyl-5,6,7,8-tetrahydroquinazolin-2-yl}-1H-indazol-1-yl)methyl]-3,5-difluorophenoxy}ethanol,
2-{3,5-difluoro-4-[(3-{4-[(3-methoxypyridin-4-yl)amino]-6,7,8,9-tetrahydro-5H-cyclohepta[d]pyrimidin-2-yl}-1H-indazol-1-yl)methyl]phenoxy}ethanol,
5-(azetidin-3-ylmethyl)-N-(2,5-dimethylpyridin-4-yl)-2-[1-(4-ethoxy-2,6-difluorobenzyl)-1H-indazol-3-yl]pyrimidin-4-amine, salt with hydrochloric acid,
5-(azetidin-3-ylmethyl)-2-[1-(4-ethoxy-2,6-difluorobenzyl)-1H-indazol-3-yl]-N-(2-methylpyrimidin-4-yl)pyrimidin-4-amine, salt with hydrochloric acid,
5-(2-aminoethyl)-N-(3-chloropyridin-4-yl)-2-[1-(4-ethoxy-2,6-difluorobenzyl)-1H-indazol-3-yl]pyrimidin-4-amine,
5-(2-aminoethyl)-2-[1-(4-ethoxy-2,6-difluorobenzyl)-1H-indazol-3-yl]-N-(2-methylpyrimidin-4-yl)pyrimidin-4-amine, salt with hydrochloric acid,
5-(2-aminoethyl)-2-[1-(4-ethoxy-2,6-difluorobenzyl)-1H-indazol-3-yl]-N-(2-methylpyrimidin-4-yl)pyrimidin-4-amine,
N-(3-chloropyridin-4-yl)-2-{1-[2,6-difluoro-4-(2-methoxyethoxy)benzyl]-1H-indazol-3-yl}-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(2-methylpyrimidin-4-yl)-6,7-dihydro-5H-pyrrolo[3,4-d]pyrimidin-4-amine, salt with hydrochloric acid,
2-{1-[2,6-difluoro-4-(2-methoxyethoxy)benzyl]-1H-indazol-3-yl}-N-(2-methylpyrimidin-4-yl)-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(2,5-dimethylpyridin-4-yl)-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(pyrimidin-4-yl)-6,7-dihydro-5H-pyrrolo[3,4-d]pyrimidin-4-amine, salt with hydrochloric acid,
2-{1-[2,6-difluoro-4-(2-methoxyethoxy)benzyl]-1H-indazol-3-yl}-N-(pyrimidin-4-yl)-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(3-methoxypyridin-4-yl)-6,7-dihydro-5H-pyrrolo[3,4-d]pyrimidin-4-amine,
5-[1-(2,2-difluoroethyl)piperidin-4-yl]-2-[1-(4-ethoxy-2,6-difluorobenzyl)-1H-indazol-3-yl]-N-(3-methoxypyridin-4-yl)pyrimidin-4-amine,
2-{1-[2,6-difluoro-4-(2-methoxyethoxy)benzyl]-1H-indazol-3-yl}-N-(2-methylpyrimidin-4-yl)-6,7-dihydro-5H-cyclopenta[d]pyrimidin-4-amine,
2-{1-[2,6-difluoro-4-(2-methoxyethoxy)benzyl]-1H-indazol-3-yl}-N-(pyrimidin-4-yl)-6,7-dihydro-5H-cyclopenta[d]pyrimidin-4-amine,
2-{1-[2,6-difluoro-4-(2-methoxyethoxy)benzyl]-1H-indazol-3-yl}-N-(2,5-dimethylpyridin-4-yl)-6,7-dihydro-5H-cyclopenta[d]pyrimidin-4-amine,
2-{1-[2,6-difluoro-4-(2-methoxyethoxy)benzyl]-1H-indazol-3-yl}-N-(5-fluoro-2-methylpyridin-4-yl)-6,7-dihydro-5H-cyclopenta[d]pyrimidin-4-amine,
N-(2,5-dimethylpyridin-4-yl)-2-[1-(4-ethoxy-2,6-difluorobenzyl)-1H-indazol-3-yl]-6,6-dimethyl-6,7-dihydro-5H-cyclopenta[d]pyrimidin-4-amine,
2-[1-(4-ethoxy-2,6-difluorobenzyl)-1H-indazol-3-yl]-6,6-dimethyl-N-(2-methylpyrimidin-4-yl)-6,7-dihydro-5H-cyclopenta[d]pyrimidin-4-amine,
2-{1-[2,6-difluoro-4-(2-methoxyethoxy)benzyl]-1H-indazol-3-yl}-6,6-dimethyl-N-(2-methylpyrimidin-4-yl)-6,7-dihydro-5H-cyclopenta[d]pyrimidin-4-amine,
N-[4-({2-[1-(4-ethoxy-2,6-difluorobenzyl)-1H-indazol-3-yl]-6,6-dimethyl-6,7-dihydro-5H-cyclopenta[d]pyrimidin-4-yl}amino)pyridin-2-yl]acetamide,
2-[1-(4-ethoxy-2,6-difluorobenzyl)-1H-indazol-3-yl]-6,6-dimethyl-N-(2-methylpyrimidin-4-yl)-5,6,7,8-tetrahydroquinazolin-4-amine,
2-[1-(4-ethoxy-2,6-difluorobenzyl)-1H-indazol-3-yl]-N-(3-methoxypyridin-4-yl)-6,6-dimethyl-5,6,7,8-tetrahydroquinazolin-4-amine,
N-(2,5-dimethylpyridin-4-yl)-2-[1-(4-ethoxy-2,6-difluorobenzyl)-1H-indazol-3-yl]-6,6-dimethyl-5,6,7,8-tetrahydroquinazolin-4-amine,
N-[4-({2-[1-(4-ethoxy-2,6-difluorobenzyl)-1H-indazol-3-yl]-6,6-dimethyl-5,6,7,8-tetrahydroquinazolin-4-yl}amino)pyridin-2-yl]acetamide,
2-{1-[2,6-difluoro-4-(2-methoxyethoxy)benzyl]-1H-indazol-3-yl}-N-(3-methoxypyridin-4-yl)-6,7,8,9-tetrahydro-5H-cyclohepta[d]pyrimidin-4-amine,
2-{1-[2,6-difluoro-4-(2-methoxyethoxy)benzyl]-1H-indazol-3-yl}-6-methyl-N-(2-methylpyrimidin-4-yl)pyrimidin-4-amine,
2-{1-[2,6-difluoro-4-(2-methoxyethoxy)benzyl]-1H-indazol-3-yl}-6-isopropyl-N-(2-methylpyrimidin-4-yl)pyrimidin-4-amine,
2-{1-[2,6-difluoro-4-(2-methoxyethoxy)benzyl]-1H-indazol-3-yl}-6-isopropyl-N-(3-methoxypyridin-4-yl)pyrimidin-4-amine,
2-{1-[2,6-difluoro-4-(2-methoxyethoxy)benzyl]-1H-indazol-3-yl}-N-(2,5-dimethylpyridin-4-yl)-6-isopropylpyrimidin-4-amine,
2-{1-[2,6-difluoro-4-(2-methoxyethoxy)benzyl]-1H-indazol-3-yl}-5-fluoro-6-methyl-N-(2-methylpyrimidin-4-yl)pyrimidin-4-amine,
2-{1-[2,6-difluoro-4-(2-methoxyethoxy)benzyl]-1H-indazol-3-yl}-N-(3-methoxypyridin-4-yl)-5,6-dimethylpyrimidin-4-amine,
2-{1-[2,6-difluoro-4-(2-methoxyethoxy)benzyl]-1H-indazol-3-yl}-N-(3-methoxypyridin-4-yl)-6-methylpyrimidin-4-amine
2-{1-[2,6-difluoro-4-(2-methoxyethoxy)benzyl]-1H-indazol-3-yl}-6-methyl-N-[3-(trifluoro-methoxy)pyridin-4-yl]pyrimidin-4-amine,
2-[1-(4-ethoxy-2,6-difluorobenzyl)-1H-indazol-3-yl]-6-methyl-N-[3-(trifluoromethoxy)pyridin-4-yl]pyrimidin-4-amine,
4-({2-[1-(4-ethoxy-2,6-difluorobenzyl)-1H-indazol-3-yl]-6-methylpyrimidin-4-yl}amino)pyridin-3-ol,
2-[1-(4-ethoxy-2,6-difluorobenzyl)-1H-indazol-3-yl]-N-(3-methoxypyridin-4-yl)-6-(trifluoromethyl)pyrimidin-4-amine,
N-(2,5-dimethylpyridin-4-yl)-2-[1-(4-ethoxy-2,6-difluorobenzyl)-1H-indazol-3-yl]-6-(trifluoromethyl)pyrimidin-4-amine,
2-[1-(4-ethoxy-2,6-difluorobenzyl)-1H-indazol-3-yl]-N-(2-methylpyrimidin-4-yl)-6-(trifluoromethyl)pyrimidin-4-amine,
6-(2,4-dimethyl-1,3-thiazol-5-yl)-2-[1-(4-ethoxy-2,6-difluorobenzyl)-1H-indazol-3-yl]-N-(2-methylpyrimidin-4-yl)pyrimidin-4-amine,
6-(2,4-dimethyl-1,3-thiazol-5-yl)-2-[1-(4-ethoxy-2,6-difluorobenzyl)-1H-indazol-3-yl]-N-(pyrimidin-4-yl)pyrimidin-4-amine,
2-[1-(4-ethoxy-2,6-difluorobenzyl)-1H-indazol-3-yl]-N-(pyrimidin-4-yl)-6-[(2rac)-tetrahydrofuran-2-yl]pyrimidin-4-amine,
2-[1-(4-ethoxy-2,6-difluorobenzyl)-1H-indazol-3-yl]-N-(2-methylpyrimidin-4-yl)-6-[(2rac)-tetrahydrofuran-2-yl]pyrimidin-4-amine,
2-[1-(4-ethoxy-2,6-difluorobenzyl)-1H-indazol-3-yl]-N-(3-methoxypyridin-4-yl)-6-[(2rac)-tetrahydrofuran-2-yl]pyrimidin-4-amine,
2-[1-(4-ethoxy-2,6-difluorobenzyl)-1H-indazol-3-yl]-N-(2-methylpyrimidin-4-yl)-6-(tetrahydro-2H-pyran-4-yl)pyrimidin-4-amine,
N-[4-({2-[1-(4-ethoxy-2,6-difluorobenzyl)-1H-indazol-3-yl]-6-(tetrahydro-2H-pyran-4-yl)pyrimidin-4-yl}amino)pyridin-2-yl]acetamide,
2-[1-(4-ethoxy-2,6-difluorobenzyl)-1H-indazol-3-yl]-N-(3-methoxypyridin-4-yl)-6-(tetrahydro-2H-pyran-4-yl)pyrimidin-4-amine,
2-[1-(4-ethoxy-2,6-difluorobenzyl)-1H-indazol-3-yl]-N-(pyrimidin-4-yl)-6-(tetrahydro-2H-pyran-4-yl)pyrimidin-4-amine,
2-[1-(4-ethoxy-2,6-difluorobenzyl)-1H-indazol-3-yl]-6-(1-methyl-1H-pyrazol-3-yl)-N-(2-methylpyrimidin-4-yl)pyrimidin-4-amine,
2-[1-(4-ethoxy-2,6-difluorobenzyl)-1H-indazol-3-yl]-N-(3-methoxypyridin-4-yl)-6-(1-methyl-1H-pyrazol-3-yl)pyrimidin-4-amine,
2-[1-(4-ethoxy-2,6-difluorobenzyl)-1H-indazol-3-yl]-N-(pyrimidin-4-yl)-6-(1,3-thiazol-2-yl)pyrimidin-4-amine,
2-[1-(4-ethoxy-2,6-difluorobenzyl)-1H-indazol-3-yl]-N-(2-methylpyrimidin-4-yl)-6-(1,3-thiazol-2-yl)pyrimidin-4-amine,
2-{1-[2,6-difluoro-4-(2-methoxyethoxy)benzyl]-1H-indazol-3-yl}-N-(2-methylpyrimidin-4-yl)-7,8-dihydro-5H-pyrano[4,3-d]pyrimidin-4-amine,
2-{1-[2,6-difluoro-4-(2-methoxyethoxy)benzyl]-1H-indazol-3-yl}-7,7-dimethyl-N-(2-methylpyrimidin-4-yl)-5,6,7,8-tetrahydroquinazolin-4-amine,
N-(3-chloropyridin-4-yl)-2-{1-[2,6-difluoro-4-(2-methoxyethoxy)benzyl]-1H-indazol-3-yl}-7,7-dimethyl-5,6,7,8-tetrahydroquinazolin-4-amine,
2-{1-[2,6-difluoro-4-(2-methoxyethoxy)benzyl]-1H-indazol-3-yl}-N-(3-methoxypyridin-4-yl)-7,7-dimethyl-5,6,7,8-tetrahydroquinazolin-4-amine,
N-{4-[(2-{1-[2,6-difluoro-4-(2-methoxyethoxy)benzyl]-1H-indazol-3-yl}-7,7-dimethyl-5,6,7,8-tetrahydroquinazolin-4-yl)amino]pyridin-2-yl}acetamide,
2-{1-[2,6-difluoro-4-(2-methoxyethoxy)benzyl]-1H-indazol-3-yl}-N-(2,5-dimethylpyridin-4-yl)-7,7-dimethyl-5,6,7,8-tetrahydroquinazolin-4-amine,
2-[1-(4-ethoxy-2,6-difluorobenzyl)-1H-indazol-3-yl]-N-(3-methoxypyridin-4-yl)-5-(piperidin-4-yl)pyrimidin-4-amine,
2-[1-(4-ethoxy-2,6-difluorobenzyl)-1H-indazol-3-yl]-N-(2-methylpyridin-4-yl)-5-(piperidin-4-yl)pyrimidin-4-amine,
N-(2,5-dimethylpyridin-4-yl)-2-[1-(4-ethoxy-2,6-difluorobenzyl)-1H-indazol-3-yl]-5-(piperidin-4-yl)pyrimidin-4-amine,
N-(4-{[2-{1-[2,6-difluoro-4-(2-methoxyethoxy)benzyl]-1H-indazol-3-yl}-5-(piperidin-4-yl)pyrimidin-4-yl]amino}pyridin-2-yl)acetamide,
N-(3-chloropyridin-4-yl)-2-{1-[2,6-difluoro-4-(2-methoxyethoxy)benzyl]-1H-indazol-3-yl}-5-(piperidin-4-yl)pyrimidin-4-amine,
2-{1-[2,6-difluoro-4-(2-methoxyethoxy)benzyl]-1H-indazol-3-yl}-N-(2-methylpyrimidin-4-yl)-5-(piperidin-4-yl)pyrimidin-4-amine,
2-[1-(4-ethoxy-2,6-difluorobenzyl)-1H-indazol-3-yl]-N-(3-methoxypyridin-4-yl)-5-[1-(2,2,2-trifluoroethyl)piperidin-4-yl]pyrimidin-4-amine,
2-[1-(4-ethoxy-2,6-difluorobenzyl)-1H-indazol-3-yl]-N-(2-methylpyridin-4-yl)-5-[1-(2,2,2-trifluoroethyl)piperidin-4-yl]pyrimidin-4-amine,
N-(2,5-dimethylpyridin-4-yl)-2-[1-(4-ethoxy-2,6-difluorobenzyl)-1H-indazol-3-yl]-5-[1-(2,2,2-trifluoroethyl)piperidin-4-yl]pyrimidin-4-amine,
N-{4-[(2-{1-[2,6-difluoro-4-(2-methoxyethoxy)benzyl]-1H-indazol-3-yl}-5-[1-(2,2,2-tri-fluoroethyl)piperidin-4-yl]pyrimidin-4-yl)amino]pyridin-2-yl}acetamide,
N-(3-chloropyridin-4-yl)-2-{1-[2,6-difluoro-4-(2-methoxyethoxy)benzyl]-1H-indazol-3-yl}-5-[1-(2,2,2-trifluoroethyl)piperidin-4-yl]pyrimidin-4-amine,
N-(3-chloropyridin-4-yl)-2-{1-[2,6-difluoro-4-(2-methoxyethoxy)benzyl]-1H-indazol-3-yl}-5-[1-(3,3,3-trifluoropropyl)piperidin-4-yl]pyrimidin-4-amine,
2-{1-[2,6-difluoro-4-(2-methoxyethoxy)benzyl]-1H-indazol-3-yl}-N-(2-methylpyrimidin-4-yl)-5-[1-(2,2,2-trifluoroethyl)piperidin-4-yl]pyrimidin-4-amine,
5-[1-(2,2-difluoroethyl)piperidin-4-yl]-2-{1-[2,6-difluoro-4-(2-methoxyethoxy)benzyl]-1H-indazol-3-yl}-N-(2-methylpyrimidin-4-yl)pyrimidin-4-amine,
5-[1-(cyclopropylmethyl)piperidin-4-yl]-2-{1-[2,6-difluoro-4-(2-methoxyethoxy)benzyl]-1H-indazol-3-yl}-N-(2-methylpyrimidin-4-yl)pyrimidin-4-amine,
2-[1-(4-ethoxy-2,6-difluorobenzyl)-1H-indazol-3-yl]-N-(2-methylpyrimidin-4-yl)-5-{2-[(2,2,2-trifluoroethyl)amino]ethyl}pyrimidin-4-amine,
N-(3-chloropyridin-4-yl)-2-[1-(4-ethoxy-2,6-difluorobenzyl)-1H-indazol-3-yl]-5-{2-[(2,2,2-trifluoroethyl)amino]ethyl}pyrimidin-4-amine,
N-{4-[(2-[1-(4-ethoxy-2,6-difluorobenzyl)-1H-indazol-3-yl]-5-{2-[(2,2,2-trifluoroethyl)-amino]ethyl}pyrimidin-4-yl)amino]pyridin-2-yl}acetamide,
2-{1-[2,6-difluoro-4-(2-methoxyethoxy)benzyl]-1H-indazol-3-yl}-N-(2-methylpyrimidin-4-yl)-6-(2,2,2-trifluoroethyl)-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(2,5-dimethylpyridin-4-yl)-6-(2,2,2-trifluoroethyl)-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(pyrimidin-4-yl)-6-(2,2,2-trifluoroethyl)-6,7-dihydro-5H-pyrrolo[3,4-d]pyrimidin-4-amine,
2-{1-[2,6-difluoro-4-(2-methoxyethoxy)benzyl]-1H-indazol-3-yl}-N-(3-methoxypyridin-4-yl)-6-(2,2,2-trifluoroethyl)-6,7-dihydro-5H-pyrrolo[3,4-d]pyrimidin-4-amine, and
2-[1-(4-ethoxy-2,6-difluorobenzyl)-1H-indazol-3-yl]-N-(3-methoxypyridin-4-yl)-5-[1-(3,3,3-trifluoropropyl)piperidin-4-yl]pyrimidin-4-amine,
   or an N-oxide, a salt, a tautomer or a stereoisomer of said compound, or a salt of said N-oxide, tautomer or stereoisomer.

A further aspect of the invention are compounds of formula (I), wherein

V, W, Y and Z independently of each other represent CH or CR³,
or,
   V represents N, and W, Y and Z independently of each other represent CH or CR³,
or,
   W represents N, and V, Y and Z independently of each other represent CH or CR³,
or,
   V and Y represent N, and W and Z independently of each other represent CH or CR³.

Yet another aspect of the invention are compounds of formula (I) in which,

V, W, Y and Z independently of each other represent CH or CR³.

Yet another aspect of the invention are compounds of formula (I) in which,

V, W, Y represent CH and Z represents CR³.

Yet another aspect of the invention are compounds of formula (I) in which,

V, W, Z represent CH and Y represents CR³.

Yet another aspect of the invention are compounds of formula (I) in which,

Z, W, Y represent CH and V represents CR³.

Yet another aspect of the invention are compounds of formula (I) in which,

V represents N, and W, Y and Z independently of each other represent CH or CR³.

Yet another aspect of the invention are compounds of formula (I) in which,

V represents N, W represents CR³, Y and Z represent CH.

Yet another aspect of the invention are compounds of formula (I) in which,

V represents N, W and Z independently of each other represent CR³, Y represents CH.

Yet another aspect of the invention are compounds of formula (I) in which,

V represents N, W and Y independently of each other represent CR³, Z represents CH.

Yet another aspect of the invention are compounds of formula (I) in which,

W represents N, and V, Y and Z independently of each other represent CH or CR³.

Yet another aspect of the invention are compounds of formula (I) in which,

V and Y represent N, and W and Z independently of each other represent CH or CR³.

A further aspect of the invention are compounds of formula (I), wherein
R¹ and R² represent, independently of each other, hydrogen, halogen, or a group selected from:
   C₁-C₆-alkyl, C₃-C₆-cycloalkyl, C₁-C₆-haloalkyl, C₁-C₆-hydroxyalkyl,
   (C₁-C₃-alkoxy)-(C₁-C₃-alkyl)-, —(C₁-C₆-alkyl)-S—R¹⁴, —(C₁-C₆-alkyl)-S(═O)—R¹⁴,
   —(C₁-C₆-alkyl)-S(═O)₂—R¹⁴, —(C₁-C₆-alkyl)-S(═O)(═NR¹⁵)R¹⁴,
   —(C₁-C₆-alkyl)-N(R¹¹)R¹², —(C₁-C₆-alkyl)-N(R¹⁷)R¹⁸, 5- or 6-membered heteroaryl,
   a 5- to 7-membered oxygen containing heterocycloalkyl group,
   (1,3-dioxolan-2-yl)-(C₁-C₆-alkyl)-, (1,3-dioxan-2-yl)-(C₁-C₆-alkyl)-, an azetidinyl group, azetidinyl-(C₁-C₆-alkyl)-, a 5- to 7-membered heterocycloalkyl group, and
   (5- to 7-membered heterocycloalkyl)-(C₁-C₆-alkyl)-,
   in which 5- or 6-membered heteroaryl is connected to rest of the molecule via a carbon atom of the heteroaryl group,
   in which 5- to 7-membered oxygen containing heterocycloalkyl is connected to rest of the molecule via a carbon atom of the 5- to 7-membered oxygen containing heterocycloalkyl group,
   in which azetidinyl and 5- to 7-membered heterocycloalkyl are connected to the rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a substituent selected from: a hydroxy, a halogen atom, cyano, C₁-C₄-alkyl, C₁-C₄-haloalkyl, C₁-C₄-alkoxy, and C₁-C₄-haloalkoxy group,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₆-cycloalkyl, C₃-C₆-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₆-cycloalkyl,
      (C₃-C₄-cycloalkyl)methyl-C₃-C₆-cycloalkyloxy, —N(R¹¹)R¹², C(═O)OH and
      —N(H)C(═O)—(C₁-C₃-alkyl),
   wherein at least one of R¹ and R² represents a group which connects via a carbon atom of said group to the rest of the molecule,
   or,
   R¹ and R² together represent a group, which is selected from:

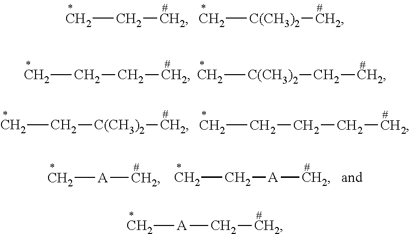

wherein A represents a group, which is selected from:
NH, N—(C₁-C₃-alkyl), N—(C₂-C₃-haloalkyl), N-benzyl, O, S, S(═O), S(═O)₂, and S(═O)(═NR¹⁵)R¹⁴,
and,
wherein * indicates the point of attachment of R¹ to the rest of the molecule,
and # indicates the point of attachment of R² to the rest of the molecule.

A further aspect of the invention are compounds of formula (I), wherein
R¹ and R² represent, independently of each other, hydrogen, halogen, or a group selected from:
   C₁-C₆-alkyl, C₃-C₆-cycloalkyl, C₁-C₆-haloalkyl, C₁-C₆-hydroxyalkyl,
   (C₁-C₃-alkoxy)-(C₁-C₃-alkyl)-, —(C₁-C₆-alkyl)-S—R¹⁴, —(C₁-C₆-alkyl)-S(═O)—R¹⁴,
   —(C₁-C₆-alkyl)-S(═O)₂—R¹⁴, —(C₁-C₆-alkyl)-S(═O)(═NR¹⁵)R¹⁴,
   —(C₁-C₆-alkyl)-N(R¹¹)R¹², —(C₁-C₆-alkyl)-N(R¹⁷)R¹⁸, 5- or 6-membered heteroaryl,
   a 5- to 7-membered oxygen containing heterocycloalkyl group,
   (1,3-dioxolan-2-yl)-(C₁-C₆-alkyl)-, (1,3-dioxan-2-yl)-(C₁-C₆-alkyl)-, an azetidinyl group, azetidinyl-(C₁-C₆-alkyl)-, a 5- to 7-membered heterocycloalkyl group, and
   (5- to 7-membered heterocycloalkyl)-(C₁-C₆-alkyl)-,
   in which 5- or 6-membered heteroaryl is connected to rest of the molecule via a carbon atom of the heteroaryl group,
   in which 5- to 7-membered oxygen containing heterocycloalkyl is connected to rest of the molecule via a carbon atom of the 5- to 7-membered oxygen containing heterocycloalkyl group,
   in which azetidinyl and 5- to 7-membered heterocycloalkyl are connected to the rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a substituent selected from: a hydroxy, a halogen atom, cyano, C₁-C₄-alkyl, C₁-C₄-haloalkyl, C₁-C₄-alkoxy, and C₁-C₄-haloalkoxy group,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₆-cycloalkyl, C₃-C₆-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₆-cycloalkyl,
      (C₃-C₄-cycloalkyl)methyl-C₃-C₆-cycloalkyloxy, —N(R¹¹)R¹², C(═O)OH and
      —N(H)C(═O)—(C₁-C₃-alkyl),
   wherein at least one of R¹ and R² represents a group which connects via a carbon atom of said group to the rest of the molecule.

A further aspect of the invention are compounds of formula (I), wherein
R¹ and R² together represent a group, which is selected from:

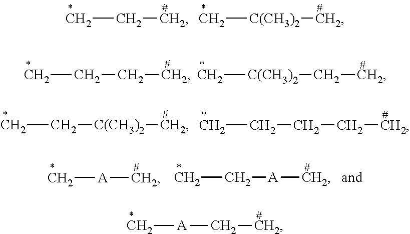

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and ^{#} indicates the point of attachment of R² to the rest of the molecule.

A further aspect of the invention are compounds of formula (I), wherein
R¹ and R² represent, independently of each other hydrogen, halogen, or a group selected from:
   C₁-C₅-alkyl, C₃-C₆-cycloalkyl, C₁-C₅-haloalkyl, C₁-C₅-hydroxyalkyl,
   (C₁-C₃-alkoxy)-(C₁-C₃-alkyl)-, —(C₁-C₅-alkyl)-S—R¹⁴, —(C₁-C₅-alkyl)-S(═O)—R¹⁴,
   —(C₁-C₅-alkyl)-S(═O)₂—R¹⁴, —(C₁-C₅-alkyl)-S(═O)(═NR¹⁵)R¹⁴,
   —(C₁-C₅-alkyl)-N(R¹¹)R¹², —(C₁-C₅-alkyl)-N(R¹⁷)R¹⁸, 5- or 6-membered heteroaryl,
   a 5- to 7-membered oxygen containing heterocycloalkyl group,
   (1,3-dioxolan-2-yl)-(C₁-C₅-alkyl)-, (1,3-dioxan-2-yl)-(C₁-C₅-alkyl)-, an azetidinyl group, azetidinyl-(C₁-C₅-alkyl)-, a 5- to 7-membered heterocycloalkyl group, and
   (5- to 7-membered heterocycloalkyl)-(C₁-C₅-alkyl)-,
   in which 5- or 6-membered heteroaryl is connected to rest of the molecule via a carbon atom of the heteroaryl group,
   in which 5- to 7-membered oxygen containing heterocycloalkyl is connected to rest of the molecule via a carbon atom of the 5- to 7-membered oxygen containing heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a methyl group,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₄-cycloalkyl, C₃-C₄-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₄-cycloalkyl,
      (C₃-C₄-cycloalkyl)methyl-C₃-C₄-cycloalkyloxy, —N(R¹¹)R¹², —C(═O)OH and —N(H)C(═O)—(C₁-C₃-alkyl),
   wherein at least one of R¹ and R² represents a group which connects via a carbon atom of said group to the rest of the molecule,
   or,
   R¹ and R² together represent a group, which is selected from:

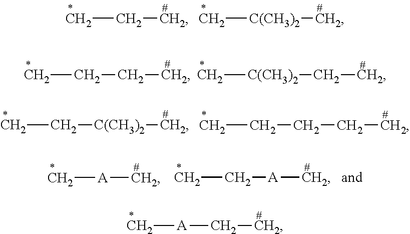

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and # indicates the point of attachment of R² to the rest of the molecule.

A further aspect of the invention are compounds of formula (I), wherein
R¹ and R² represent, independently of each other hydrogen, halogen, or a group selected from:
   C₁-C₅-alkyl, C₃-C₆-cycloalkyl, C₁-C₅-haloalkyl, C₁-C₅-hydroxyalkyl,
   (C₁-C₃-alkoxy)-(C₁-C₃-alkyl)-, —(C₁-C₅-alkyl)-S—R¹⁴, —(C₁-C₅-alkyl)-S(═O)—R¹⁴,
   —(C₁-C₅-alkyl)-S(═O)₂—R¹⁴, —(C₁-C₅-alkyl)-S(═O)(═NR¹⁵)R¹⁴,
   —(C₁-C₅-alkyl)-N(R¹¹)R¹², —(C₁-C₅-alkyl)-N(R¹⁷)R¹⁸, 5- or 6-membered heteroaryl,
   a 5- to 7-membered oxygen containing heterocycloalkyl group,
   (1,3-dioxolan-2-yl)-(C₁-C₅-alkyl)-, (1,3-dioxan-2-yl)-(C₁-C₅-alkyl)-, an azetidinyl group, azetidinyl-(C₁-C₅-alkyl)-, a 5- to 7-membered heterocycloalkyl group, and
   (5- to 7-membered heterocycloalkyl)-(C₁-C₅-alkyl)-,
   in which 5- or 6-membered heteroaryl is connected to rest of the molecule via a carbon atom of the heteroaryl group,
   in which 5- to 7-membered oxygen containing heterocycloalkyl is connected to rest of the molecule via a carbon atom of the 5- to 7-membered oxygen containing heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a methyl group,
   wherein azetidinyl is optionally substituted with a substituent selected from:
      hydroxy, a halogen atom, cyano, C₁-C₄-alkyl, C₁-C₄-haloalkyl, C₁-C₄-alkoxy, C₁-C₄-haloalkoxy, (C₁-C₃-alkoxy)-(C₁-C₄-alkyl)-,
      C₃-C₄-cycloalkyl, C₃-C₄-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₄-cycloalkyl,
      (C₃-C₄-cycloalkyl)methyl-C₃-C₄-cycloalkyloxy, —N(R¹¹)R¹², —C(═O)OH and —N(H)C(═O)—(C₁-C₃-alkyl),
   wherein at least one of R¹ and R² represents a group which connects via a carbon atom of said group to the rest of the molecule.

A further aspect of the invention are compounds of formula (I), wherein
R¹ represents hydrogen, halogen, or a group selected from:
   C₁-C₅-alkyl, C₁-C₅-haloalkyl, C₁-C₅-hydroxyalkyl,
   (C₁-C₃-alkoxy)-(C₁-C₃-alkyl)-, —(C₁-C₅-alkyl)-S—R¹⁴,
   —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, 5-membered heteroaryl, and a 5- or 6-membered oxygen containing heterocycloalkyl group,
   in which 5-membered heteroaryl is connected to rest of the molecule via a carbon atom of the heteroaryl group,
   wherein 5-membered heteroaryl is optionally substituted, one or two times, with a methyl group,
   wherein at least one of R¹ and R² represents a group which connects via a carbon atom of said group to the rest of the molecule.

A further aspect of the invention are compounds of formula (I), wherein
R¹ represents hydrogen, or a group selected from:
   C₁-C₃-alkyl, trifluoromethyl, C₁-C₂-hydroxyalkyl, methoxymethyl-, —(CH₂)—S—R¹⁴,
   —(CH₂)—S(═O)₂—R¹⁴, —(CH₂)—N(R¹⁷)R¹⁸,5-membered heteroaryl, and a 5- or 6-membered oxygen containing heterocycloalkyl group,
   in which 5-membered heteroaryl is connected to rest of the molecule via a carbon atom of the heteroaryl group,
   which 5-membered heteroaryl group is selected from:
   1,3-thiazolyl and pyrazolyl, which groups are optionally substituted, one or two times with a methyl group,
   which 5- or 6-membered oxygen containing heterocycloalkyl group is selected from:
   tetrahydrofuranyl and tetrahydro-2H-pyranyl,
   wherein at least one of R¹ and R² represents a group which connects via a carbon atom of said group to the rest of the molecule.

A further aspect of the invention are compounds of formula (I), wherein
R² represents hydrogen, halogen, or a group selected from:
   C₁-C₅-alkyl, C₃-C₆-cycloalkyl, C₁-C₅-haloalkyl, C₁-C₅-hydroxyalkyl,
   (C₁-C₃-alkoxy)-(C₁-C₃-alkyl)-, —(C₁-C₅-alkyl)-S—R¹⁴, —(C₁-C₅-alkyl)-S(═O)—R¹⁴,
   —(C₁-C₅-alkyl)-S(═O)₂—R¹⁴, —(C₁-C₅-alkyl)-S(═O)(═NR¹⁵)R¹⁴,
   —(C₁-C₅-alkyl)-N(R¹¹)R¹², —(C₁-C₅-alkyl)-N(R¹⁷)R¹⁸,
   (1,3-dioxolan-2-yl)-(C₁-C₅-alkyl)-, (1,3-dioxan-2-yl)-(C₁-C₅-alkyl)-, an azetidinyl group, azetidinyl-(C₁-C₅-alkyl)-, a 5- to 7-membered heterocycloalkyl group, and
   (5- to 7-membered heterocycloalkyl)-(C₁-C₅-alkyl)-,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      a halogen atom, C₁-C₄-alkyl, and C₁-C₄-haloalkyl,
      or being substituted with two halogen atoms,
   wherein 5- to 7-membered heterocycloalkyl is optionally substituted, one, two, three, four or five times, identically or differently, with a substituent selected from:
      halogen, C₁-C₄-alkyl, C₁-C₄-haloalkyl and cyclopropylmethyl-,
   wherein at least one of R¹ and R² represents a group which connects via a carbon atom of said group to the rest of the molecule.

A further aspect of the invention are compounds of formula (I), wherein
R² represents hydrogen, halogen, or a group selected from:
   C₁-C₃-alkyl, cyclopropyl, C₁-C₃-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₃-alkyl)-N(R¹¹)R¹²,
   —(C₁-C₅-alkyl)-N(R¹⁷)R¹⁸, (1,3-dioxolan-2-yl)-(C₁-C₅-alkyl)-, an azetidinyl group, azetidinyl-(C₁-C₅-alkyl)-, and a 5- to 7-membered heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      C₁-C₄-haloalkyl,
   wherein 5- to 7-membered heterocycloalkyl is optionally substituted with a substituent selected from:
      C₁-C₄-haloalkyl and cyclopropylmethyl-,
   wherein at least one of R¹ and R² represents a group which connects via a carbon atom of said group to the rest of the molecule.

A further aspect of the invention are compounds of formula (I), wherein
R² represents hydrogen, fluorine, chlorine, or a group selected from:
   C₁-C₂-alkyl, cyclopropyl, —(CH₂)₃OH, methoxy-(C₂-C₃-alkyl)-, —(C₁-C₃-alkyl)-S—R¹⁴,
   —(C₁-C₃-alkyl)-S(═O)—R¹⁴, —(C₁-C₃-alkyl)-S(═O)₂—R¹⁴, —(CH₂)₂—S(═O)(═NR¹⁵)R¹⁴,
   —(CH₂)₃—S(═O)(═NR¹⁵)R¹⁴, —(C₁-C₄-alkyl)-N(R¹¹)R¹², —(C₁-C₄-alkyl)-N(R¹⁷)R¹⁸,
   (1,3-dioxolan-2-yl)-(CH₂)—, an azetidinyl group, azetidinyl-(CH₂)—, and a piperidinyl group,
   in which azetidinyl and piperidinyl are connected to rest of the molecule via a carbon atom of the azetidinyl ring or via a carbon atom of the piperidinyl ring,
   wherein azetidinyl is optionally substituted with a 2,2,2-trifluoroethyl group,
   wherein piperidinyl is optionally substituted with a substituent selected from:

2,2,2-trifluoroethyl, 2,2-difluoroethyl, 3,3,3-trifluoropropyl and cyclopropylmethyl-,
wherein at least one of R¹ and R² represents a group which connects via a carbon atom of said group to the rest of the molecule.

A further aspect of the invention are compounds of formula (I), wherein R¹ and R² together represent a group, which is selected from:

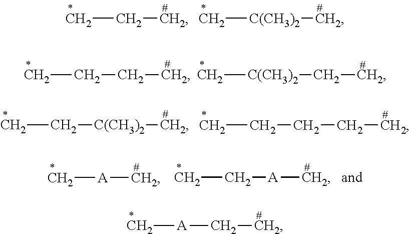

wherein A represents a group, which is selected from:
NH, N—(C₁-C₃-alkyl), N-(2,2,2-trifluoroethyl), N-benzyl, O, S, and S(═O)₂, and,
wherein * indicates the point of attachment of R¹ to the rest of the molecule,
and # indicates the point of attachment of R² to the rest of the molecule.

A further aspect of the invention are compounds of formula (I), wherein R¹ and R² together represent a group, which is selected from: R¹ and R² together represent a group, which is selected from:

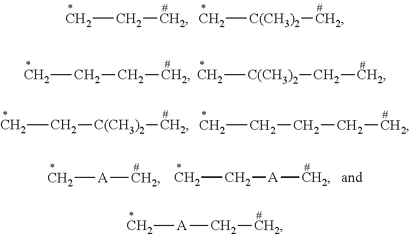

wherein A represents a group, which is selected from:
NH, N-ethyl, N-(2,2,2-trifluoroethyl), N-benzyl, O, S, and S(═O)₂,
and,
wherein * indicates the point of attachment of R¹ to the rest of the molecule,
and # indicates the point of attachment of R² to the rest of the molecule.

A further aspect of the invention are compounds of formula (I), wherein
R³ represents, independently of each other, halogen or a group selected from:
   hydroxy, C₁-C₃ alkyl, C₃-C₄-cycloalkyl, C₁-C₃ haloalkyl, C₁-C₃-alkoxy,
   C₁-C₃-haloalkoxy, —N(H)C(═O)—(C₁-C₃-alkyl), —N(H)C(═O)—(C₃-C₄-cycloalkyl),
   —C(═O)OH, —C(═O)NH₂, and —S(═O)₂—(C₁-C₃-alkyl).

A further aspect of the invention are compounds of formula (I), wherein
R³ represents fluorine, chlorine or a group selected from:
   hydroxy, methyl, cyclopropyl, difluoromethyl, trifluoromethyl, methoxy,
   trifluoromethoxy, —N(H)C(═O)—CH₃, —N(H)C(═O)-cyclopropyl, —C(═O)OH,
   —C(═O)NH₂, and —S(═O)₂—CH₃.

A further aspect of the invention are compounds of formula (I), wherein
R⁶ represents a group selected from:
   hydroxy, C₁-C₂-alkoxy, (2-hydroxyethyl)-O—, and 2-methoxy-ethoxy-.

A further aspect of the invention are compounds of formula (I), wherein
R¹ and R² represent, independently of each other, hydrogen, halogen, or a group selected from:
   C₁-C₆-hydroxyalkyl, (C₁-C₃-alkoxy)-(C₁-C₃-alkyl)-,
   —(C₁-C₆-alkyl)-S(═O)—R¹⁴, —(C₁-C₆-alkyl)-S(═O)₂—R¹⁴,
   —(C₁-C₆-alkyl)-S(═O)(═NR¹⁵)R¹⁴, —(C₁-C₆-alkyl)-N(R¹¹)R¹²,
   —(C₁-C₆-alkyl)-N(R¹⁷)R¹⁸, (1,3-dioxolan-2-yl)-(C₁-C₆-alkyl)-,
   (1,3-dioxan-2-yl)-(C₁-C₆-alkyl)-, an azetidinyl group, azetidinyl-(C₁-C₆-alkyl)
   a 5- to 7-membered heterocycloalkyl group, and
   (5- to 7-membered heterocycloalkyl)-(C₁-C₆-alkyl)-,
   in which azetidinyl and 5- to 7-membered heterocycloalkyl are connected to the rest of the molecule via a carbon atom of the azetidinyl ring or via a carbon atom of the 5- to 7-membered heterocycloalkyl ring,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₆-cycloalkyl, C₃-C₆-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl, C₁-C₄-alkoxy,
      C₁-C₄-haloalkoxy, (C₁-C₃-alkoxy)-(C₁-C₄-alkyl)-, C₃-C₆-cycloalkyl,
      C₃-C₆-cycloalkyloxy, —N(R¹¹)R¹², C(═O)OH and —N(H)C(═O)—(C₁-C₃-alkyl),
   wherein at least one of R¹ and R² represents a group which connects via a carbon atom of said group to the rest of the molecule,
   or,
   R¹ and R² together represent a group, which is selected from:

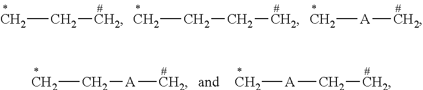

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and ^{#} indicates the point of attachment of R² to the rest of the molecule.

Yet another aspect of the invention are compounds of formula (I) in which,
R¹ and R² represent, independently of each other, hydrogen, halogen, or a group selected from:
   C₁-C₆-alkyl, C₁-C₆-haloalkyl, C₁-C₆-hydroxyalkyl,
   (C₁-C₃-alkoxy)-(C₁-C₃-alkyl)-, —(C₁-C₆-alkyl)-S—R¹⁴,
   —(C₁-C₆-alkyl)-S(═O)—R¹⁴, —(C₁-C₆-alkyl)-S(═O)₂—R¹⁴,
   —(C₁-C₆-alkyl)-S(═O)(═NR¹⁵)R¹⁴,
   —(C₁-C₆-alkyl)-N(R¹¹)R¹², —(C₁-C₆-alkyl)-N(R¹⁷)R¹⁸,
   (1,3-dioxolan-2-yl)-(C₁-C₆-alkyl)-, (1,3-dioxan-2-yl)-(C₁-C₆-alkyl)-, an azetidinyl group, azetidinyl-(C₁-C₆-alkyl)-, a 5- to 7-membered heterocycloalkyl group, and
   (5- to 7-membered heterocycloalkyl)-(C₁-C₆-alkyl)-,
   in which azetidinyl and 5- to 7-membered heterocycloalkyl are connected to the rest of the molecule via a carbon atom of the azetidinyl ring or via a carbon atom of the 5- to 7-membered heterocycloalkyl ring,
   wherein azetidinyl is optionally substituted with a substituent selected from:
      hydroxy, a halogen atom, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₆-cycloalkyl, C₃-C₆-cycloalkyloxy, —N(R¹¹)R¹², and
      —N(H)C(═O)—(C₁-C₃-alkyl),
      or being substituted with two halogen atoms,
   wherein 5- to 7-membered heterocycloalkyl is optionally substituted, one, two, three, four or five times, identically or differently, with a substituent selected from:
      hydroxy, halogen, cyano, C₁-C₄-alkyl, C₁-C₄-haloalkyl,
      C₁-C₄-alkoxy, C₁-C₄-haloalkoxy, (C₁-C₃-alkoxy)-(C₁-C₄-alkyl)-,
      C₃-C₆-cycloalkyl, C₃-C₆-cycloalkyloxy, —N(R¹¹)R¹², C(═O)OH and
      —N(H)C(═O)—(C₁-C₃-alkyl),
   wherein at least one of R¹ and R² represents a group which connects via a carbon atom of said group to the rest of the molecule.

Yet another aspect of the invention are compounds of formula (I) in which,
R¹ and R² together represent a group, which is selected from:

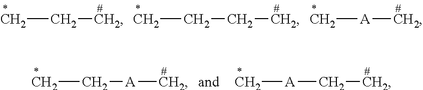

wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and ^{#} indicates the point of attachment of R² to the rest of the molecule.

A further aspect of the invention are compounds of formula (I), wherein
R¹ and R² represent, independently of each other, hydrogen, halogen, or a group selected from:
   C₁-C₆-alkyl, C₁-C₆-hydroxyalkyl, (C₁-C₃-alkoxy)-(C₁-C₃-alkyl)-,
   —(C₁-C₆-alkyl)-S—R¹⁴, —(C₁-C₆-alkyl)-S(═O)—R¹⁴, —(C₁-C₆-alkyl)-S(═O)₂—R¹⁴,
   —(C₁-C₆-alkyl)-S(═O)(═NR¹⁵)R¹⁴, —(C₁-C₆-alkyl)-N(R¹¹)R¹²,
   —(C₁-C₆-alkyl)-N(R¹⁷)R¹⁸, (1,3-dioxolan-2-yl)-(C₁-C₆-alkyl)-, an azetidinyl group,
   azetidinyl-(C₁-C₆-alkyl)-, a 5- to 7-membered heterocycloalkyl group, and
   (5- to 7-membered heterocycloalkyl)-(C₁-C₆-alkyl)-,
   in which azetidinyl and 5- to 7-membered heterocycloalkyl are connected to the rest of the molecule via a carbon atom of the azetidinyl ring or via a carbon atom of the 5- to 7-membered heterocycloalkyl ring,
   wherein azetidinyl is optionally substituted with a substituent selected from:
      C₁-C₄-haloalkyl,
   wherein 5- to 7-membered heterocycloalkyl is optionally substituted, one, time, identically or differently, with a substituent selected from:
      C₁-C₄-haloalkyl,
   wherein at least one of R¹ and R² represents a group which connects via a carbon atom of said group to the rest of the molecule,
   or,
   R¹ and R² together represent a group, which is selected from:

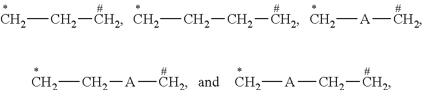

wherein A represents a group, which is selected from:
NH, N—(C₁-C₃-alkyl), N-benzyl, 0, S and S(═O)₂,
and,
wherein * indicates the point of attachment of R¹ to the rest of the molecule,
and ^{#} indicates the point of attachment of R² to the rest of the molecule.

Yet another aspect of the invention are compounds of formula (I) in which,
R¹ represents hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹², and
   —(C₁-C₅-alkyl)-N(R¹⁷)R¹⁸, wherein at least one of R¹ and R² represents a group which connects via a carbon atom of said group to the rest of the molecule.

Yet another aspect of the invention are compounds of formula (I) in which,

R¹ represents hydrogen, or a group selected from:
C₁-C₅-alkyl, C₁-C₅-hydroxyalkyl, (C₁-C₃-alkoxy)-(C₁-C₃-alkyl)-, —(C₁-C₅-alkyl)-S—R¹⁴,
—(C₁-C₅-alkyl)-S(═O)₂—R¹⁴ and —(C₁-C₅-alkyl)-N(R¹⁷)R¹⁸,
wherein at least one of R¹ and R² represents a group which connects via a carbon atom of said group to the rest of the molecule.

Yet another aspect of the invention are compounds of formula (I) in which,

R¹ represents hydrogen, or a group selected from:
C₁-C₃-alkyl, C₁-C₂-hydroxyalkyl, methoxymethyl-, —(CH₂)—S—R¹⁴, —(CH₂)—S(═O)₂—R¹⁴, and —(CH₂)—N(R¹⁷)R¹⁸,
wherein at least one of R¹ and R² represents a group which connects via a carbon atom of said group to the rest of the molecule.

Yet another aspect of the invention are compounds of formula (I) in which,

R¹ represents hydrogen, or a group selected from:
C₁-C₃-alkyl, C₁-C₂-hydroxyalkyl, methoxymethyl-, and —(CH₂)—N(R¹⁷)R¹⁸,
wherein at least one of R¹ and R² represents a group which connects via a carbon atom of said group to the rest of the molecule.

A further aspect of the invention are compounds of formula (I), wherein
R² represents hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-S—R¹⁴, —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, (1,3-dioxolan-2-yl)-(C₁-C₅-alkyl)-,
   (1,3-dioxan-2-yl)-(C₁-C₅-alkyl)-, an azetidinyl group, azetidinyl-(C₁-C₅-alkyl)-,
   a 5- to 7-membered heterocycloalkyl group, and
   (5- to 7-membered heterocycloalkyl)-(C₁-C₅-alkyl)
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      a halogen atom, C₁-C₄-alkyl, and C₁-C₄-haloalkyl,
      or being substituted with two halogen atoms,
   wherein 5- to 7-membered heterocycloalkyl is optionally substituted, one, two, three, four or five times, identically or differently, with a substituent selected from:
      halogen, C₁-C₄-alkyl, and C₁-C₄-haloalkyl,
   wherein at least one of R¹ and R² represents a group which connects via a carbon atom of said group to the rest of the molecule.

Yet another aspect of the invention are compounds of formula (I) in which,
R² represents hydrogen, halogen, or a group selected from:
   C₁-C₅-alkyl, C₁-C₅-haloalkyl, C₁-C₅-hydroxyalkyl, (C₁-C₃-alkoxy)-(C₁-C₃-alkyl)-,
   —(C₁-C₅-alkyl)-N(R¹¹)R¹², —(C₁-C₅-alkyl)-N(R¹⁷)R¹⁸,
   (1,3-dioxolan-2-yl)-(C₁-C₅-alkyl)-, (1,3-dioxan-2-yl)-(C₁-C₅-alkyl)-, an azetidinyl group, azetidinyl-(C₁-C₅-alkyl)-, a 5- to 7-membered heterocycloalkyl group, and
   (5- to 7-membered heterocycloalkyl)-(C₁-C₅-alkyl)-,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
      a halogen atom, C₁-C₄-alkyl, and C₁-C₄-haloalkyl,
      or being substituted with two halogen atoms,
      wherein 5- to 7-membered heterocycloalkyl is optionally substituted, one, two, three, four or five times, identically or differently, with a substituent selected from:
      halogen, C₁-C₄-alkyl, and C₁-C₄-haloalkyl,
   wherein at least one of R¹ and R² represents a group which connects via a carbon atom of said group to the rest of the molecule.

Yet another aspect of the invention are compounds of formula (I) in which,
R² represents hydrogen, halogen, or a group selected from:
   C₁-C₅-alkyl, C₁-C₅-hydroxyalkyl, (C₁-C₃-alkoxy)-(C₁-C₃-alkyl)-, —(C₁-C₅-alkyl)-S—R¹⁴,
   —(C₁-C₅-alkyl)-S(═O)—R¹⁴, —(C₁-C₅-alkyl)-S(═O)₂—R¹⁴,
   —(C₁-C₅-alkyl)-S(═O)(═NR¹⁵)R¹⁴, —(C₁-C₅-alkyl)-N(R¹¹)R¹²,
   —(C₁-C₅-alkyl)-N(R¹⁷)R¹⁸, (1,3-dioxolan-2-yl)-(C₁-C₅-alkyl)-, an azetidinyl group,
   azetidinyl-(C₁-C₅-alkyl)-, and a 5- to 7-membered heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from: C₁-C₄-haloalkyl,
   wherein 5- to 7-membered heterocycloalkyl is optionally substituted, one, time, identically or differently, with a substituent selected from:
      C₁-C₄-haloalkyl,
   wherein at least one of R¹ and R² represents a group which connects via a carbon atom of said group to the rest of the molecule.

Yet another aspect of the invention are compounds of formula (I) in which,
R² represents hydrogen, halogen, or a group selected from:
   C₁-C₅-alkyl, C₁-C₅-hydroxyalkyl, (C₁-C₃-alkoxy)-(C₁-C₃-alkyl)-, —(C₁-C₅-alkyl)-N(R¹¹)R¹², —(C₁-C₅-alkyl)-N(R¹⁷)R¹⁸, (1,3-dioxolan-2-yl)-(C₁-C₅-alkyl)-, an azetidinyl group, azetidinyl-(C₁-C₅-alkyl)-, and a 5- to 7-membered heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein azetidinyl is optionally substituted with a substituent selected from:
   C₁-C₄-haloalkyl,
   wherein 5- to 7-membered heterocycloalkyl is optionally substituted, one, time, identically or differently, with a substituent selected from:
      C₁-C₄-haloalkyl,
   wherein at least one of R¹ and R² represents a group which connects via a carbon atom of said group to the rest of the molecule.

Yet another aspect of the invention are compounds of formula (I) in which,
R² represents hydrogen, fluorine, chlorine, or a group selected from:
   C₁-C₂-alkyl, —(CH₂)₃OH, methoxy-(C₂-C₃-alkyl)-, —(C₁-C₃-alkyl)-S—R¹⁴,
   —(C₁-C₃-alkyl)-S(═O)—R¹⁴, —(C₁-C₃-alkyl)-S(═O)₂—R¹⁴, —(CH₂)₃—S(═O)(═NR¹⁵)R¹⁴,
   —(C₁-C₄-alkyl)-N(R¹¹)R¹², —(C₁-C₄-alkyl)-N(R¹⁷)R¹⁸, (1,3-dioxolan-2-yl)-(CH₂)—, an azetidinyl group, azetidinyl-(CH₂)—, and a piperidinyl group,
   in which azetidinyl and piperidinyl are connected to rest of the molecule via a carbon atom of the azetidinyl ring or via a carbon atom of the piperidinyl ring,
   wherein azetidinyl is optionally substituted with a 2,2,2-trifluoroethyl group,
   wherein piperidinyl is optionally substituted with a 2,2,2-trifluoroethyl group,
   wherein at least one of R¹ and R² represents a group which connects via a carbon atom of said group to the rest of the molecule.

A further aspect of the invention are compounds of formula (I), wherein
R¹ and R² together represent a group, which is selected from:

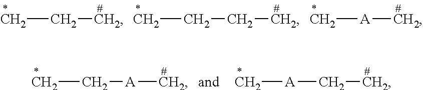

wherein A represents a group, which is selected from:
NH, N—(C₁-C₃-alkyl), N—(C₂-C₃-haloalkyl), N-benzyl, O, S, S(═O), and S(═O)₂,
and,
wherein * indicates the point of attachment of R¹ to the rest of the molecule,
and # indicates the point of attachment of R² to the rest of the molecule.

Yet another aspect of the invention are compounds of formula (I) in which,
R¹ and R² together represent a group, which is selected from:

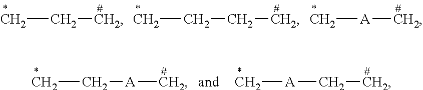

wherein A represents a group, which is selected from:
NH, N—(C₁-C₃-alkyl), N-benzyl, O, S, and S(═O)₂,
and,
wherein * indicates the point of attachment of R¹ to the rest of the molecule,
and # indicates the point of attachment of R² to the rest of the molecule.

A further aspect of the invention are compounds of formula (I), wherein
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₁-C₃-alkyl)-(C₁-C₃-alkoxy), —N(H)C(═O)-phenyl,
   —N(H)C(═O)—(C₃-C₄-cycloalkyl), —N(H)C(═O)—(C₁-C₃-hydroxyalkyl),
   —C(═O)OH, —C(═O)NH₂, —S(═O)₂—(C₁-C₃-alkyl),
   —N(H)C(═O)—(C₁-C₃-alkyl)-(C₃-C₄-cycloalkyl), and —N(H)C(═O)N(H)R¹⁹,
      said —N(H)C(═O)-phenyl being optionally substituted at the phenyl ring, one, two or three times, identically or differently, with a substituent selected from: halogen, hydroxy, cyano, C₁-C₄-alkyl, C₁-C₄-haloalkyl, C₁-C₄-alkoxy,
      C₁-C₄-haloalkoxy, C₃-C₆-cycloalkyl, and C₃-C₆-cycloalkyloxy,
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy.

Yet another aspect of the invention are compounds of formula (I) in which,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   —N(H)C(═O)—(C₁-C₃-alkyl), —N(H)C(═O)—(C₃-C₄-cycloalkyl), —C(═O)OH,
   —C(═O)NH₂, —S(═O)₂—(C₁-C₃-alkyl).

Yet another aspect of the invention are compounds of formula (I) in which,
R³ represents, independently of each other, halogen or a group selected from:
   hydroxy, C₁-C₃-alkyl, C₃-C₄-cycloalkyl, C₁-C₃-haloalkyl, C₁-C₃-alkoxy,
   C₁-C₃-haloalkoxy, —N(H)C(═O)H, —N(H)C(═O)—(C₁-C₃-alkyl),
   —N(H)C(═O)—(C₃-C₄-cycloalkyl), —N(H)C(═O)—(C₁-C₃-hydroxyalkyl), —C(═O)OH,
   —C(═O)NH₂, and —S(═O)₂—(C₁-C₃-alkyl),
      said —N(H)C(═O)—(C₃-C₄-cycloalkyl) being optionally substituted at the C₃-C₄-cycloalkyl ring with a substituent selected from:
      fluorine, chlorine, trifluoromethyl, and methoxy.

Yet another aspect of the invention are compounds of formula (I) in which,
R³ represents fluorine, chlorine or a group selected from:
   hydroxy, methyl, cyclopropyl, difluoromethyl, trifluoromethyl, methoxy,
   —N(H)C(═O)—CH₃, —N(H)C(═O)-cyclopropyl, —C(═O)OH, —C(═O)NH₂, and
   —S(═O)₂—CH₃.

A further aspect of the invention are compounds of formula (I), wherein
R⁴ and R⁵ represent, independently of each other, hydrogen or fluorine.

A further aspect of the invention are compounds of formula (I), wherein
R⁶ represents a group selected from:
   hydroxy, C₁-C₆-alkyl, C₁-C₆-haloalkyl, C₁-C₆-hydroxyalkyl,
   (C₁-C₃-alkoxy)-(C₁-C₆-alkyl)-, C₃-C₆-cycloalkyl, (C₃-C₆-cycloalkyl)-(C₁-C₃-alkyl) C₁-C₆-alkoxy, C₁-C₆-haloalkoxy, (C₂-C₆-hydroxyalkyl)-O—,
   (C₁-C₃-alkoxy)-(C₂-C₆-alkoxy)-, C₃-C₆-cycloalkyloxy,
   (C₃-C₆-cycloalkyl)-(C₁-C₃-alkoxy)-, and R⁸,
      wherein said hydroxyalkyl groups are optionally substituted with one, two or three halogen atoms selected from:
fluorine, and chlorine.

Yet another aspect of the invention are compounds of formula (I) in which,
R⁶ represents a group selected from:
   hydroxy, C₁-C₆-alkoxy, (C₂-C₆-hydroxyalkyl)-O—, (C₁-C₃-alkoxy)-(C₂-C₆-alkoxy)-.

Yet another aspect of the invention are compounds of formula (I) in which,
R⁶ represents a group selected from:
   hydroxy, C₁-C₆-alkyl, C₁-C₆-haloalkyl, C₁-C₆-hydroxyalkyl,
   (C₁-C₃-alkoxy)-(C₁-C₆-alkyl)-, C₃-C₆-cycloalkyl, (C₃-C₆-cycloalkyl)-(C₁-C₃-alkyl) C₁-C₆-alkoxy, C₁-C₆-haloalkoxy, (C₂-C₆-hydroxyalkyl)-O—,
   (C₁-C₃-alkoxy)-(C₂-C₆-alkoxy)-, C₃-C₆-cycloalkyloxy,
   (C₃-C₆-cycloalkyl)-(C₁-C₃-alkoxy)-,
      wherein said hydroxyalkyl groups are optionally substituted with one, two or three halogen atoms selected from:
fluorine, and chlorine.

Yet another aspect of the invention are compounds of formula (I) in which,
R⁶ represents a group selected from R⁸.

Yet another aspect of the invention are compounds of formula (I) in which,
R⁶ represents a group selected from:
   hydroxy, C₁-C₄-alkoxy, C₁-C₄-haloalkoxy, (C₂-C₄-hydroxyalkyl)-O—, (C₁-C₃-alkoxy)-(C₂-C₃-alkoxy)-, and R⁸.

Yet another aspect of the invention are compounds of formula (I) in which,
R⁶ represents R⁸.

Yet another aspect of the invention are compounds of formula (I) in which,
R⁶ represents a group selected from:
hydroxy, C₁-C₄-alkoxy, (C₂-C₄-hydroxyalkyl)-O—, and (C₁-C₃-alkoxy)-(C₂-C₃-alkoxy)-.

Yet another aspect of the invention are compounds of formula (I) in which,
R⁶ represents a group selected from:
   hydroxy, C₁-C₂-alkoxy, (2-hydroxyethyl)-O—, and methoxy-ethoxy-.

A further aspect of the invention are compounds of formula (I), wherein
R⁷ represents hydrogen, or a 2-hydroxyethyl group.

A further aspect of the invention are compounds of formula (I), wherein
R² and R⁷ together represent a

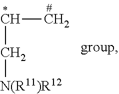

wherein * indicates the point of attachment of R² to the rest of the molecule.
and # indicates the point of attachment of R⁷ to the rest of the molecule.

A further aspect of the invention are compounds of formula (I), wherein
R⁸ represents a group selected from:
   —(C₂-C₆-alkyl)-OC(═O)—C(H)(R⁹)—N(H)C(═O)—C(H)(R¹⁰)—NH₂, and
   —O—(C₂-C₆-alkyl)-OC(═O)—C(H)(R⁹)—N(H)C(═O)—C(H)(R¹⁰)—NH₂,
      wherein said C₂-C₆-alkyl groups are optionally substituted with one, two or three halogen atoms selected from:
      fluorine, and chlorine.

Yet another aspect of the invention are compounds of formula (I) in which,
R⁸ represents a group selected from:
   —(C₂-C₄-alkyl)-OC(═O)—C(H)(R⁹)—N(H)C(═O)—C(H)(R¹⁰)—NH₂, and
   —O—(C₂-C₄-alkyl)-OC(═O)—C(H)(R⁹)—N(H)C(═O)—C(H)(R¹⁰)—NH₂.

A further aspect of the invention are compounds of formula (I), wherein
R⁹ and R¹⁰ independently of each other represent hydrogen (glycine) or a group selected from:
   —CH₃ (alanine), —C(H)(CH₃)₂ (valine), —(CH₂)₂CH₃ (norvaline), —CH₂C(H)(CH₃)₂ (leucine), —C(H)(CH₃)CH₂CH₃ (isoleucine), —(CH₂)₃CH₃ (norleucine), —C(CH₃)₃ (2-tert-butylglycine), benzyl (phenylalanine), 4-hydroxybenzyl (tyrosine), —(CH₂)₃NH₂ (ornithine), —(CH₂)₄NH₂ (lysine), —(CH₂)₂C(H)(OH)CH₂NH₂ (hydroxylysine), —CH₂OH (serine), —(CH₂)₂OH (homoserine), —C(H)(OH)CH₃ (threonine), —(CH₂)₃N(H)C(═NH)NH₂ (arginine), —(CH₂)₃N(H)C(═O)NH₂ (citrulline), —CH₂C(═O)NH₂ (asparagine), —CH₂C(═O)OH (aspartic acid), —(CH₂)₂C(═O)OH (glutamic acid), —(CH₂)₂C(═O)NH₂ (glutamine), —CH₂SH (cysteine), —(CH₂)₂SH (homocysteine), —(CH₂)₂SCH₃ (methionine), —CH₂SCH₃ (S-methylcysteine), (1H-imidazol-4-yl)methyl-(histidine), (1H-indol-3-yl)methyl-(thryptophan), —CH₂NH₂ (2,3-diaminopropanoic acid), and —(CH₂)₂NH₂ (2,4-diaminobutanoic acid).

Yet another aspect of the invention are compounds of formula (I) in which,
R⁹ and R¹⁰ independently of each other represent a group selected from:
   —CH₃ (alanine), —C(H)(CH₃)₂ (valine), —(CH₂)₂CH₃ (norvaline), —(CH₂)₃NH₂ (ornithine), —(CH₂)₄NH₂ (lysine), and —(CH₂)₃N(H)C(═NH)NH₂ (arginine).

A further aspect of the invention are compounds of formula (I), wherein
R¹¹ and R¹² independently of each other represents hydrogen or a group selected from:
   C₁-C₃-alkyl, C₂-C₃-haloalkyl, —C(═O)H, and —C(═O)—(C₁-C₃-alkyl).

Yet another aspect of the invention are compounds of formula (I) in which,
R¹¹ and R¹² independently of each other represents hydrogen or a group selected from:
   C₂-C₃-haloalkyl, and —C(═O)H.

Yet another aspect of the invention are compounds of formula (I) in which,
R¹¹ and R¹² independently of each other represents hydrogen or a group selected from:
   2,2,2-trifluoroethyl, and C(═O)H.

A further aspect of the invention are compounds of formula (I), wherein
R¹⁴ represents, independently of each other, a group selected from:
   C₁-C₆ alkyl, C₃-C₆-cycloalkyl, phenyl, and benzyl,
      wherein said group is optionally substituted with one, two or three substituents, identical or different, selected from the group of hydroxy, halogen, and —NR¹¹R¹².

Yet another aspect of the invention are compounds of formula (I) in which,
R¹⁴ represents, independently of each other, a group selected from:
C₁-C₄ alkyl, C₃-C₄-cycloalkyl.

Yet another aspect of the invention are compounds of formula (I) in which,
R¹⁴ represents, independently of each other, a group selected from:
   C₁-C₄-alkyl, preferably represents methyl.

A further aspect of the invention are compounds of formula (I), wherein
R¹⁵ represents hydrogen or a group selected from:
   cyano, and —C(═O)R¹⁶.

Yet another aspect of the invention are compounds of formula (I) in which,
R¹⁵ represents hydrogen or —C(═O)R¹⁶.

A further aspect of the invention are compounds of formula (I), wherein
R¹⁶ represents a group selected from:
   C₁-C₆-alkyl, and C₁-C₆-haloalkyl.

Yet another aspect of the invention are compounds of formula (I) in which,
R¹⁶ represents C₁-C₃-haloalkyl.

Yet another aspect of the invention are compounds of formula (I) in which,
R¹⁶ represents a trifluoromethyl group.

A further aspect of the invention are compounds of formula (I), wherein
R¹⁷ and R¹⁸ together with the nitrogen to which they are attached form
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₆-cycloalkyl, C₃-C₆-cycloalkyloxy,
      hydroxy, halogen, cyano, —C(═O)H, (C₁-C₄-alkyl)-C(═O)—, —N(R¹¹)R¹²,
      —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, C₁-C₄-alkoxy, C₁-C₄-haloalkoxy,
      (C₁-C₃-alkoxy)-(C₁-C₄-alkyl)-, C₃-C₆-cycloalkyl, C₃-C₆-cycloalkyloxy,
      hydroxy, a halogen atom, cyano, —C(═O)H, (C₁-C₄-alkyl)-C(═O)—,
      —N(R¹¹)R¹², —(C₁-C₄-alkyl)-N(R¹¹)R¹², —C(═O)N(R¹¹)R¹²,
      —(C₁-C₄-alkyl)-C(═O)N(R¹¹)R¹², and —C(═O)OH.

Yet another aspect of the invention are compounds of formula (I) in which,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom or heteroatom containing group selected from O, NH, S, S(═O), and S(═O)₂,
   said azetidinyl group being optionally substituted with a substituent selected from:
      C₁-C₄-alkyl, C₁-C₄-haloalkyl, halogen,
      or being substituted with two halogen atoms,
   said 5- to 7-membered heterocycloalkyl group being optionally substituted, one, two, three, four or five times, identically or differently, with a substituent selected from:
      C₁-C₄-alkyl, C₁-C₄-haloalkyl, and a halogen atom.

Yet another aspect of the invention are compounds of formula (I) in which,
R¹⁷ and R¹⁸ together with the nitrogen to which they are attached form:
   an azetidinyl group or a 5- to 7-membered heterocycloalkyl group,
   said 5- to 7-membered heterocycloalkyl group optionally containing one additional heteroatom or heteroatom containing group selected from 0,
   said azetidinyl group being optionally substituted with a substituent selected from: two halogen atoms,
   said 5- to 7-membered heterocycloalkyl group being optionally substituted, two, identically or differently, with a substituent selected from:
      halogen.

Yet another aspect of the invention are compounds of formula (I) in which,
R¹⁷ and R¹⁸ together with the nitrogen to which they are attached form:
   an azetidinyl group or a 6-membered heterocycloalkyl group,
   said 6-membered heterocycloalkyl group optionally containing one additional heteroatom selected from 0,
   said azetidinyl group being substituted with two fluorine atoms,
   said 6-membered heterocycloalkyl group being optionally substituted with two fluorine atoms.

A further aspect of the invention are compounds of formula (I), wherein
R¹⁹ represents hydrogen or a group selected from:
   C₁-C₃-alkyl, C₁-C₃-haloalkyl, C₂-C₃-hydroxyalkyl, C₃-C₄-cycloalkyl,
   (C₃-C₄-cycloalkyl)-(C₁-C₃-alkyl) and (C₁-C₃-alkoxy)-(C₂-C₃-alkyl)-.

In accordance with a further aspect, the invention relates to compounds of formula (I),

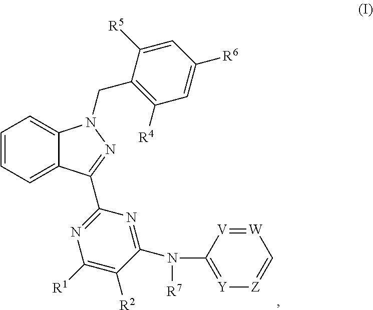

in which
V, W, Y and Z independently of each other represent CH or CR³,
   or,
V represents N, and W, Y and Z independently of each other represent CH or CR³,
   or,
W represents N, and V, Y and Z independently of each other represent CH or CR³,
R¹ and R² represent, independently of each other, hydrogen, halogen, or a group selected from:
   C₁-C₆-alkyl, C₃-C₆-cycloalkyl, C₁-C₆-haloalkyl, C₁-C₆-hydroxyalkyl,
   —(C₁-C₆-alkyl)-N(R¹¹)R¹², —(C₁-C₆-alkyl)-N(R¹⁷)R¹⁸, 5- or 6-membered heteroaryl, a 5- to 7-membered oxygen containing heterocycloalkyl group, an azetidinyl group,
   azetidinyl-(C₁-C₆-alkyl)-, and a 5- to 7-membered heterocycloalkyl group,
   in which 5- or 6-membered heteroaryl is connected to rest of the molecule via a carbon atom of the heteroaryl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein 5- or 6-membered heteroaryl is optionally substituted, one or two times, with a methyl group,
   wherein 5- to 7-membered heterocycloalkyl is optionally substituted with a substituent selected from:
      C₁-C₄-haloalkyl, and (C₃-C₄-cycloalkyl)methyl-,
   wherein at least one of R¹ and R² represents a group which connects via a carbon atom of said group to the rest of the molecule,
   or,
   R¹ and R² together represent a group, which is selected from:

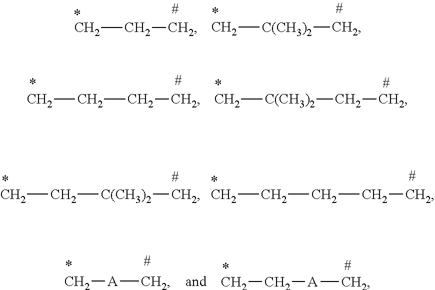

wherein A represents a group, which is selected from:
NH, N—(C₁-C₃-alkyl), N—(C₂-C₃-haloalkyl), and O,
and,
wherein * indicates the point of attachment of R¹ to the rest of the molecule,
and # indicates the point of attachment of R² to the rest of the molecule,
R³ represents, independently of each other, halogen or a group selected from:
   hydroxy, C₁-C₃-alkyl, C₁-C₃-alkoxy, C₁-C₃-haloalkoxy, and
   —N(H)C(═O)—(C₁-C₃-alkyl),
R⁴ and R⁵ represent a fluorine atom,
R⁶ represents a group selected from:
   C₁-C₆-alkoxy, (C₂-C₆-hydroxyalkyl)-O—, and (C₁-C₃-alkoxy)-(C₂-C₆-alkoxy)-,
R⁷ represents hydrogen,
R¹¹ and R¹² independently of each other represents hydrogen or a C₂-C₃-haloalkyl group,
R¹⁷ and R¹⁸ together with the nitrogen to which they are attached form
   a 5- to 7-membered heterocycloalkyl group,
   said 5- to 7-membered heterocycloalkyl group optionally containing one additional heteroatom or heteroatom containing group selected from 0,
      or an N-oxide, a salt, a tautomer or a stereoisomer of said compound, or a salt of said N-oxide, tautomer or stereoisomer.

In accordance with a further aspect, the invention relates to compounds of formula (I),

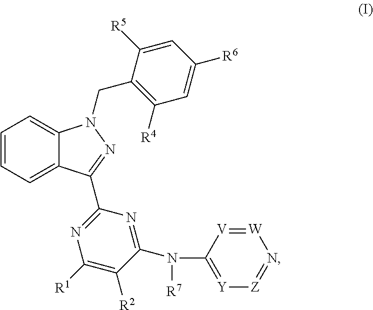

in which
V, W, Y and Z independently of each other represent CH or CR³,
   or,
V represents N, and W, Y and Z independently of each other represent CH or CR³,
   or,
W represents N, and V, Y and Z independently of each other represent CH or CR³,
R¹ represents hydrogen, or a group selected from:
   C₁-C₅-alkyl, C₁-C₅-haloalkyl, —(C₁-C₅-alkyl)-N(R¹⁷)R¹⁸, 5-membered heteroaryl, and a 5- or 6-membered oxygen containing heterocycloalkyl group,
   in which 5-membered heteroaryl is connected to rest of the molecule via a carbon atom of the heteroaryl group,
   wherein 5-membered heteroaryl is optionally substituted, one or two times, with a methyl group,
R² represents hydrogen, halogen, or a group selected from:
   C₁-C₅-alkyl, cyclopropyl, —(C₁-C₅-alkyl)-N(R¹¹)R¹², azetidinyl-(C₁-C₅-alkyl)-, and a 5- to 7-membered heterocycloalkyl group,
   in which azetidinyl and 5- to 7-membered heterocycloalkyl are connected to rest of the molecule via a carbon atom of the azetidinyl ring or via a carbon atom of the 5- to 7-membered heterocycloalkyl ring,
   wherein 5- to 7-membered heterocycloalkyl is optionally substituted with a substituent selected from:
      C₁-C₄-haloalkyl and cyclopropylmethyl-,
   wherein at least one of R¹ and R² represents a group which connects via a carbon atom of said group to the rest of the molecule,
   or,
   R¹ and R² together represent a group, which is selected from:

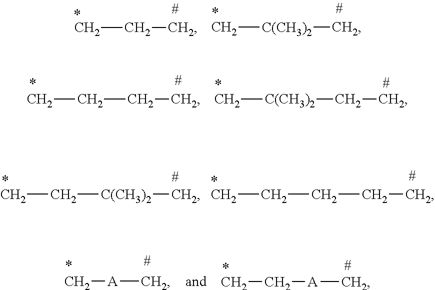

wherein A represents a group, which is selected from:
NH, N—(C₁-C₃-alkyl), N—(C₂-C₃-haloalkyl), and O,
and,
wherein * indicates the point of attachment of R¹ to the rest of the molecule,
and # indicates the point of attachment of R² to the rest of the molecule,
R³ represents, independently of each other, halogen or a group selected from:
   hydroxy, C₁-C₃-alkyl, C₁-C₃-alkoxy, C₁-C₃-haloalkoxy, and
   —N(H)C(═O)—(C₁-C₃-alkyl),
R⁴ and R⁵ represent a fluorine atom,
R⁶ represents a group selected from:
   C₁-C₄-alkoxy, (C₂-C₄-hydroxyalkyl)-O—, and (C₁-C₃-alkoxy)-(C₂-C₃-alkoxy)-,
R⁷ represents hydrogen,
R¹¹ and R¹² independently of each other represents hydrogen or a C₂-C₃-haloalkylgroup,
R¹⁷ and R¹⁸ together with the nitrogen to which they are attached form a morpholine group,
   or an N-oxide, a salt, a tautomer or a stereoisomer of said compound, or a salt of said N-oxide, tautomer or stereoisomer.

In accordance with a further aspect, the invention relates to compounds of formula (I),

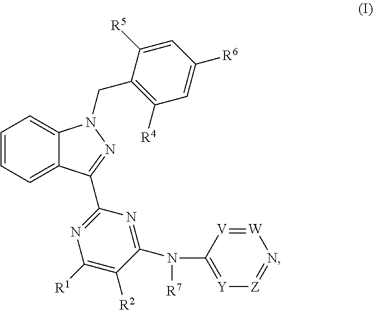

in which
V, W, Y and Z independently of each other represent CH or CR³,
   or,
V represents N, and W, Y and Z independently of each other represent CH or CR³,
   or,
W represents N, and V, Y and Z independently of each other represent CH or CR³,
R¹ represents hydrogen, or a group selected from:
   C₁-C₃-alkyl, trifluoromethyl, —(CH₂)—N(R¹⁷)R¹⁸, 5-membered heteroaryl, and a 5- or 6-membered oxygen containing heterocycloalkyl group,
   in which 5-membered heteroaryl is connected to rest of the molecule via a carbon atom of the heteroaryl group,
   which 5-membered heteroaryl group is selected from: 1,3-thiazolyl and pyrazolyl, which groups are optionally substituted, one or two times with a methyl group,
   which 5- or 6-membered oxygen containing heterocycloalkyl group is selected from:
   tetrahydrofuranyl and tetrahydro-2H-pyranyl,
R² represents hydrogen, fluorine, or a group selected from:
   methyl, cyclopropyl, —(C₁-C₂-alkyl)-N(R¹¹)R¹², azetidinyl-(CH₂)—, and a piperidinyl group,
   in which azetidinyl and piperidinyl are connected to rest of the molecule via a carbon atom of the azetidinyl ring or via a carbon atom of the piperidinyl ring,
   wherein piperidinyl is optionally substituted with a substituent selected from:
      2,2,2-trifluoroethyl, 2,2-difluoroethyl, 3,3,3-trifluoropropyl and cyclopropylmethyl-,
   wherein at least one of R¹ and R² represents a group which connects via a carbon atom of said group to the rest of the molecule,
   or,
   R¹ and R² together represent a group, which is selected from:

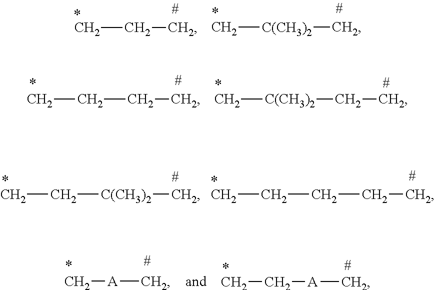

wherein A represents a group, which is selected from:
NH, N-(ethyl), N-(2,2,2-trifluoroethyl), and O,
and,
wherein * indicates the point of attachment of R¹ to the rest of the molecule,
and ^{#} indicates the point of attachment of R² to the rest of the molecule,
R³ represents, independently of each other, fluorine, chlorine or a group selected from:
   hydroxy, methyl, methoxy, trifluoromethoxy, and —N(H)C(═O)—CH₃,
R⁴ and R⁵ represent a fluorine atom,
R⁶ represents a group selected from:
   ethoxy, 2-hydroxyethoxy, and 2-methoxyethoxy,
R⁷ represents hydrogen,
R¹¹ and R¹² independently of each other represents hydrogen or a 2,2,2-trifluoroethyl group,
R¹⁷ and R¹⁸ together with the nitrogen to which they are attached form a morpholine group,
   or an N-oxide, a salt, a tautomer or a stereoisomer of said compound, or a salt of said N-oxide, tautomer or stereoisomer.

In accordance with a further aspect, the invention relates to compounds of formula (I) supra, wherein:
V and Y represent CH,
W represents CH or CR³,
Z represents CH or C(methyl),
R¹ represents hydrogen,
R² represents a piperidinyl group,
   in which piperidinyl is connected to rest of the molecule via a carbon atom of the piperidinyl ring,
   wherein piperidinyl is optionally substituted with a 2,2,2-trifluoroethyl group,
R³ represents a group selected from:
   methyl, methoxy, and —N(H)C(═O)—CH₃,
R⁴ and R⁵ represent fluorine,
R⁶ represents a group selected from:
   C₁-C₂-alkoxy,
   R⁷ represents hydrogen,
      or an N-oxide, a salt, a tautomer or a stereoisomer of said compound, or a salt of said N-oxide, tautomer or stereoisomer.

In accordance with a further aspect, the invention relates to compounds of formula (I) supra, wherein
V and Y represent CH,
W represents CR³,
Z represents CH or C(methyl),
R¹ represents hydrogen,
R² represents a piperidinyl group,
   in which piperidinyl is connected to rest of the molecule via a carbon atom of the piperidinyl ring,
   wherein piperidinyl is optionally substituted with a 2,2,2-trifluoroethyl group,
R³ represents a group selected from:
   methyl, methoxy, and —N(H)C(═O)—CH₃,
R⁴ and R⁵ represent fluorine,
R⁶ represents a group selected from:
   C₁-C₂-alkoxy,
R⁷ represents hydrogen,
   or an N-oxide, a salt, a tautomer or a stereoisomer of said compound, or a salt of said N-oxide, tautomer or stereoisomer.

One aspect of the invention are compounds of formula (I) as described in the examples, as characterized by their names in the title, as claimed in claim 6, and their structures as well as the subcombinations of all residues specifically disclosed in the compounds of the examples.

Another aspect of the present invention are the intermediates as used for their synthesis.

In accordance with yet another aspect, the present invention relates to a method of preparing a compound of general formula (I), said method comprising the step of allowing an intermediate compound of general formula (1-16):

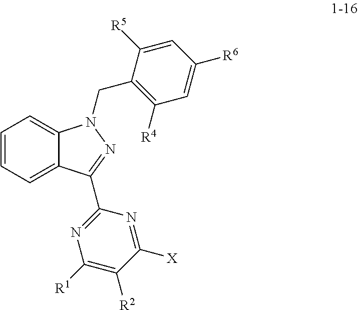

in which R¹, R², R⁴, R⁵, R⁶ are is as defined herein for the compound of formula (I), and X represents Cl or Br,
to react with a compound of the general formula (1-12),

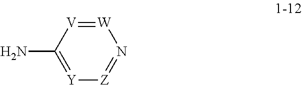

in which V, W, Y, and Z are as defined herein for the compound of formula (I),
thereby giving a compound of general formula (I):

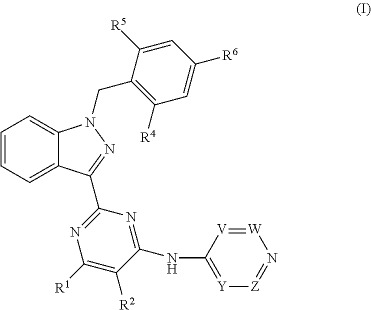

in which R¹, R², R⁴, R⁵, R⁶, V, W, Y, and Z are as defined herein for the compound of formula (I).

In particular, the present invention relates to a compound of general formula (1-16):

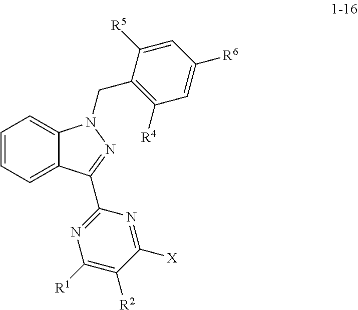

in which R¹, R², R⁴, R⁵, and R⁶ are as defined herein for the compound of formula (I) and X represents Cl or Br.

In accordance with yet another aspect, the present invention relates to the use of a compound of formula 1-16 as defined herein for the compound of formula (I), for the preparation of a compound of general formula (I)

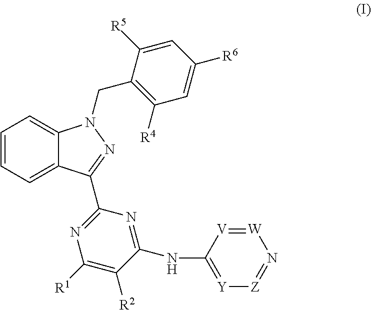

in which R¹, R², R⁴, R⁵, R⁶, V, W, Y, and Z are is as defined herein for the compound of formula (I).

A further aspect of the invention are compounds of formula (I), which are present as their salts.

Yet another aspect of the invention are compounds of formula (I) in which, the salt is a pharmaceutically acceptable salt.

It is to be understood that the present invention relates to any sub-combination within any embodiment or aspect of the present invention of compounds of general formula (I), supra.

More particularly still, the present invention covers compounds of general formula (I) which are disclosed in the Example section of this text, infra.

In accordance with another aspect, the present invention covers methods of preparing compounds of the present invention, said methods comprising the steps as described in the Experimental Section herein.

Another embodiment of the invention are compounds according to the claims as disclosed in the Claims section wherein the definitions are limited according to the preferred or more preferred definitions as disclosed below or specifically disclosed residues of the exemplified compounds and subcombinations thereof.

### Definitions

Constituents which are optionally substituted as stated herein, may be substi-tuted, unless otherwise noted, one or more times, independently from one another at any possible position. When any variable occurs more than one time in any constituent, each definition is independent. For example, whenever R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, V, W, Y and/or Z occur more than one time for any compound of formula (I) each definition of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, V, W, Y and Z is independent.

Should a constituent be composed of more than one part, e.g. C₁-C₄-alkoxy-C₁-C₄-alkyl-, the position of a possible substituent can be at any of these parts at any suitable position. A hyphen at the beginning or at the end of the constituent marks the point of attachment to the rest of the molecule. Should a ring be substituted the substitutent could be at any suitable position of the ring, also on a ring nitrogen atom if suitable.

The term “comprising” when used in the specification includes “consisting of”.

If it is referred to “as mentioned above” or “mentioned above” within the description it is referred to any of the disclosures made within the specification in any of the preceding pages.

“suitable” within the sense of the invention means chemically possible to be made by methods within the knowledge of a skilled person.

The terms as mentioned in the present text have preferably the following meanings:

The term “halogen atom”, “halo-” or “Hal-” is to be understood as meaning a fluorine, chlorine, bromine or iodine atom.

The term “C₁-C₆-alkyl” is to be understood as meaning a linear or branched, saturated, monovalent hydrocarbon group having 1, 2, 3, 4, 5, or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methylbutyl, 1-ethylpropyl, 1,2-dimethylpropyl, neo-pentyl, 1, 1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “C₁-C₆-haloalkyl” is to be understood as meaning a linear or branched, saturated, monovalent hydrocarbon group in which the term “C₁-C₆-alkyl” is defined supra, and in which one or more hydrogen atoms is replaced by a halogen atom, in identically or differently, i.e. one halogen atom being independent from another. Particularly, said halogen atom is F. Said C₁-C₆-haloalkyl group is, for example, —CF₃, —CHF₂, —CH₂F, —CF₂CF₃, —CH₂CH₂F, —CH₂CHF₂, —CH₂CF₃, —CH₂CH₂CF₃, or —CH(CH₂F)₂.

The term “C₁-C₆-alkoxy” is to be understood as meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “C₁-C₆-haloalkoxy” is to be understood as meaning a linear or branched, saturated, monovalent C₁-C₆-alkoxy group, as defined supra, in which one or more of the hydrogen atoms is replaced, in identically or differently, by a halogen atom. Particularly, said halogen atom is F. Said C₁-C₆-haloalkoxy group is, for example, —OCF₃, —OCHF₂, —OCH₂F, —OCF₂CF₃, or —OCH₂CF₃.

The term “C₁-C₆-hydroxyalkyl” is to be understood as preferably meaning a linear or branched, saturated, monovalent hydrocarbon group in which the term “C₁-C₆-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₃-C₆-cycloalkyl” is to be understood as meaning a saturated, monovalent, monocyclic hydrocarbon ring which contains 3, 4, 5 or 6 carbon atoms (“C₃-C₆-cycloalkyl”). Said C₃-C₆-cycloalkyl group is for example, a monocyclic hydrocarbon ring, e.g. a cyclopropyl, cyclobutyl, cyclopentyl or cyclohexyl ring.

The term “C₃-C₆-cycloalkyloxy” is to be understood as meaning a saturated, monovalent, monocyclic hydrocarbon group of formula —O-cycloalkyl, in which the term “cycloalkyl” is defined supra, e.g. a. a cyclopropyloxy, cyclobutyloxy, cyclopentyloxy or cyclohexyloxy group.

The term “5- to 7-membered heterocycloalkyl”, is to be understood as meaning a saturated, or partially unsaturated, monovalent, monocyclic ring which contains one N atom or one NH-group and 4 to 6 carbon atoms, wherein one carbon atom is optionally replaced by C(═O), and wherein one carbon atom is optionally replaced by a further heteroatom selected from the group consisting of N, O and S, or by a heteroatom containing group NH, S(═O) or S(═O)₂. Said heterocycloalkyl is for example, a pyrrolidinyl, piperidinyl, morpholinyl, thiomorpholinyl, piperazinyl; azepanyl, diazepanyl, or oxazepanyl.

The term “5- to 7-membered oxygen containing heterocycloalkyl”, is to be understood as meaning a saturated, monovalent, monocyclic ring which contains one 0 atom and 4 to 6 carbon atoms, wherein one carbon atom is optionally replaced by C(═O), Said oxygen containing heterocycloalkyl is for example, a tetrahydrofuranyl, tetrahydro-2H-pyranyl, or oxepanyl.

The term “heteroaryl” is understood as meaning a monovalent, monocyclic, aromatic ring system having 5 or 6 ring atoms (a “5- or 6-membered heteroaryl” group), which contains at least one ring heteroatom and optionally one, two or three further ring heteroatoms from the series N, NH, O and/or S, and which is bound via a ring carbon atom or optionally via a ring nitrogen atom (when allowed by valency).

Said heteroaryl group can be a “5-membered heteroaryl” group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a “6-membered heteroaryl” group, such as, for example, pyridinyl, pyridazinyl, pyrimidinyl, pyrazinyl or triazinyl.

In general, and unless otherwise mentioned, the heteroarylic or heteroarylenic radicals include all possible isomeric forms thereof, e.g.: tautomers and positional isomers with respect to the point of linkage to the rest of the molecule. Thus, for some illustrative non restricting example, the term pyridinyl includes pyridin-2-yl, pyridin-3-yl and pyridin-4-yl; or the term thienyl includes thien-2-yl and thien-3-yl.

The term “C₁-C₆”, as used throughout this text, e.g. in the context of the definition of “C₁-C₆-alkyl”, “C₁-C₆-haloalkyl”, “C₁-C₆-hydroxyalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, particularly C₁-C₂, C₁-C₃, C₁-C₄, C₁-C₅, C₁-C₆, more particularly C₁-C₄ in the case of “C₁-C₆-haloalkyl” or “C₁-C₆-haloalkoxy” even more particularly C₁-C₂.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence is preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention optionally contain one or more asymmetric centre, depending upon the location and nature of the various substituents desired. Asymmetric carbon atoms is present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

The compounds of the present invention optionally contain sulphur atoms which are asymmetric, such as an asymmetric sulfoxide, of structure:

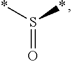

for example,
in which * indicates atoms to which the rest of the molecule can be bound. Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, the compounds of the present invention may exist as tautomers.

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulf uric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods. The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF₃COOH”, “x Na⁺”, for example, are to be understood as not a stoichiometric specification, but solely as a salt form.

The salts include water-insoluble and, particularly, water-soluble salts.

This applies analogously to cases in which synthesis intermediates or example compounds or salts thereof have been obtained, by the preparation and/or purification processes described, as solvates, such as hydrates with (if defined) unknown stoichiometric composition.

Furthermore, derivatives of the compounds of formula (I) and the salts thereof which are converted into a compound of formula (I) or a salt thereof in a biological system (bioprecursors or pro-drugs) are covered by the invention. Said biological system is e.g. a mammalian organism, particularly a human subject. The bioprecursor is, for example, converted into the compound of formula (I) or a salt thereof by metabolic processes.

As used herein, the term “in vivo hydrolysable ester” is understood as meaning an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

In the context of the properties of the compounds of the present invention the term “pharmacokinetic profile” means one single parameter or a combination thereof including permeability, bioavailability, exposure, and pharmacodynamic parameters such as duration, or magnitude of pharmacological effect, as measured in a suitable experiment. Compounds with improved pharmacokinetic profiles can, for example, be used in lower doses to achieve the same effect, may achieve a longer duration of action, or a may achieve a combination of both effects.

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered. Any such combination of a compound of formula (I) of the present invention with an anti-cancer agent as defined below is an embodiment of the invention.

The term “(chemotherapeutic) anti-cancer agents”, includes but is not limited to 1311-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporf in, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporf in, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

It has now been found, and this constitutes the basis of the present invention, that said compounds of the present invention have surprising and advantageous properties.

In particular, said compounds of the present invention have surprisingly been found to effectively inhibit Bub1 kinase and may therefore be used for the treatment or prophylaxis of diseases of uncontrolled cell growth, proliferation and/or survival, inappropriate cellular immune responses, or inappropriate cellular inflammatory responses or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Bub1 kinase, such as, for example, haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

The intermediates used for the synthesis of the compounds of claims 1-6 as described below, as well as their use for the synthesis of the compounds of claims 1-6, are one further aspect of the present invention. Preferred intermediates are the Intermediate Examples as disclosed below.

General Procedures

The compounds according to the invention can be prepared according to the following schemes 1 through 32.

The schemes and procedures described below illustrate synthetic routes to the compounds of general formula (I) of the invention and are not intended to be limiting. It is obvious to the person skilled in the art that the order of transformations as exemplified in the Schemes can be modified in various ways. The order of transformations exemplified in the Schemes is therefore not intended to be limiting. In addition, interconversion of any of the substituents, R¹, R², R⁴, R⁵, R⁶, R⁸, R¹⁰, R¹⁴, R¹⁵, V, W, Y and Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

One route for the preparation of compounds of general formula (Ia) is described in Scheme 1.

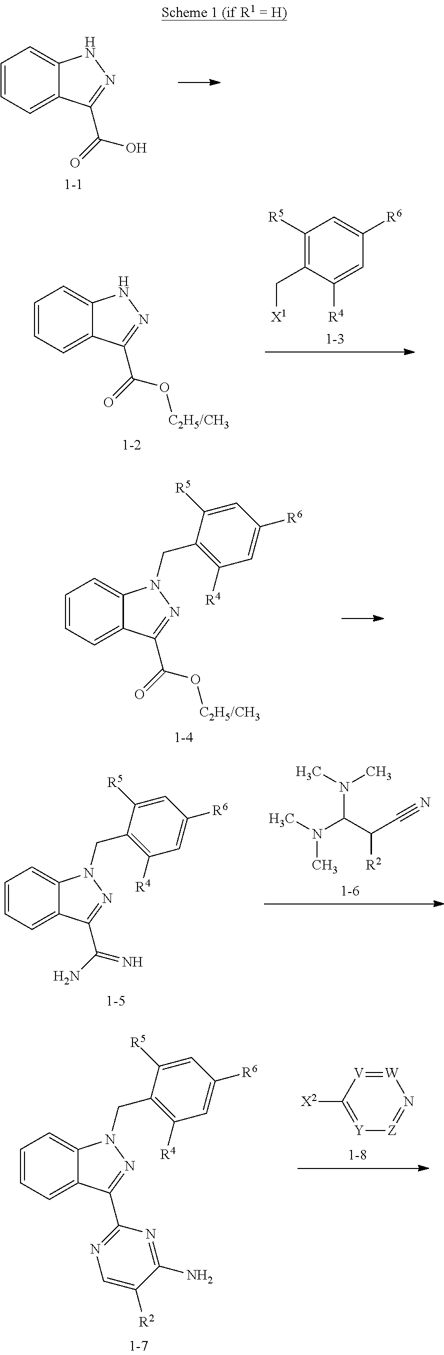

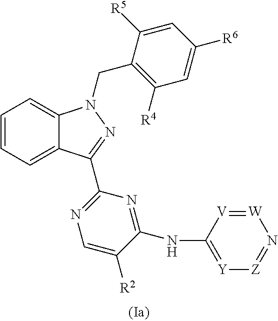

Scheme 1: Route for the preparation of compounds of general formula (Ia), wherein R², R⁴, R⁵, R⁶, V, W, Y and Z have the meaning as given for general formula (I), supra. X¹ represents F, Cl, Br, I or a sulfonate, e.g. trifluormethylsulfonate or p-toluolsulfonate, and X² represents F, Cl, Br, I, boronic acid or a boronic acid ester, such as for example 4,4,5,5-tetramethyl-2-phenyl-1,3,2-dioxaborolane (boronic acid pinacole ester). In addition, interconversion of any of the substituents, R², R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-3, 1-6 and 1-8 are either commercially available or can be prepared according to procedures available from the public domain, as understandable to the person skilled in the art. Specific examples are described in the subsequent paragraphs.

A suitably substituted 1H-indazole-3-carboxylic acid of general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

Alkyl 1H-indazole-3-carboxylate Intermediates of the general formula (1-2) can be converted to intermediates of general formula (1-4) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-4) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-5).

Intermediates of general formula (1-5) can be converted to intermediates of general formula (1-7) by reaction with a suitably substituted 3,3-bis(dimethylamino)propanenitrile of the general formula (1-6), in the presence of a suitable base, such as, for example piperidine, in a suitable solvent system, such as, for example, 3-methylbutan-1-ol, in a temperature range from room temperature to the boiling point of the respective solvent, preferably the reaction is carried out at 100° C.

Intermediates of general formula (1-7) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (Ia). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl}palladium, {1,3-bis[2,6-di(propan-2-yl)phenyl]-2,3-dihydro-1H-imidazol-2-yl}(dichloro)(3-chloropyridine-kappaN)palladium, [1,3-bis(2, 6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium(II), dichloro(di-μ-chloro)bis[1,3-bis(2,6-di-iso-propylphenyl) imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:

racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (Ia).

Alternatively intermediates of general formula (1-7) can be reacted with a suitable substituted six membered heterocycle of the general formula (1-8), such as for example 4-fluoropyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish compounds of general formula (Ia).

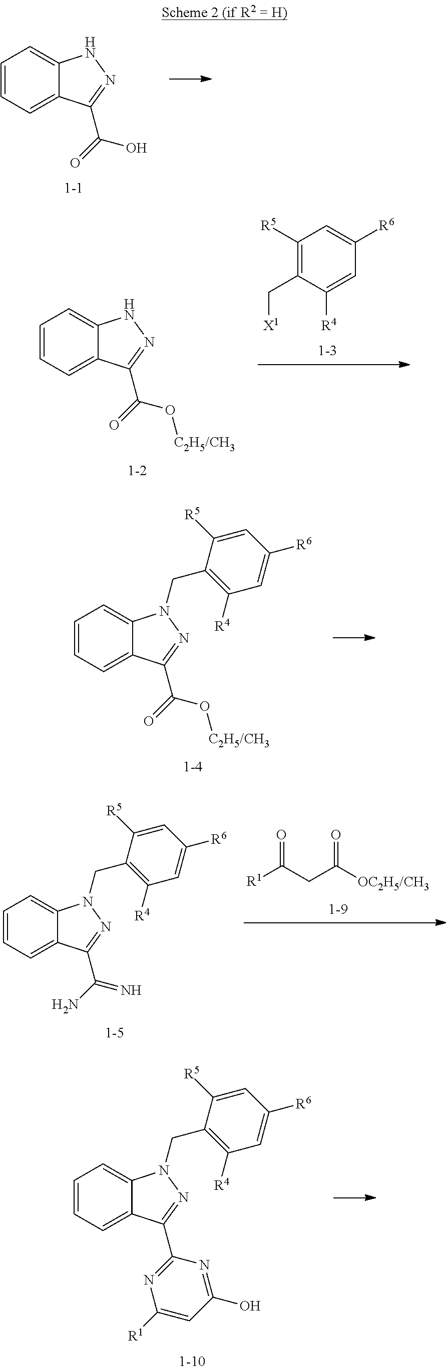

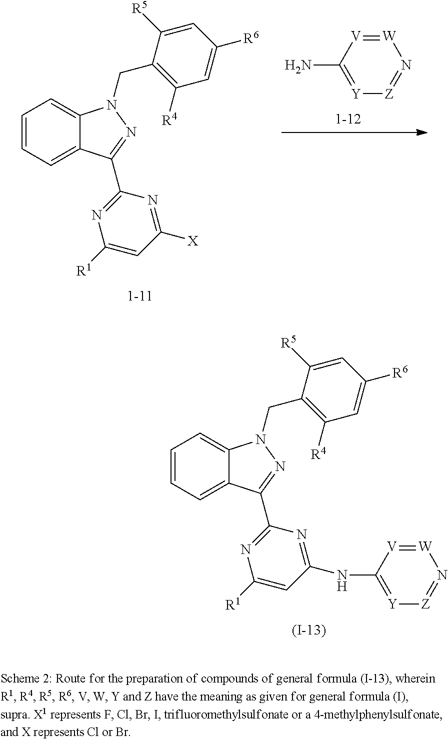

In addition, interconversion of any of the substituents R¹, R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents.

Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-3, 1-9 and 1-12 are either commercially available or can be prepared according to procedures available from the public domain, as understandable to the person skilled in the art. Specific examples are described in the subsequent paragraphs.

A suitably substituted 1H-indazole-3-carboxylic acid of general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

A suitably substituted 1H-indazole-3-carboxylic acid of general formula (1-1) can be reacted with methanol or ethanol in the presence of peptide coupling agent, such as, for example, HATU (N-[(dimethylamino)(3H-[1,2,3]triazolo[4,5-b]pyridin-3-yloxy)methylidene]-N-methylmethanaminium hexafluorophosphate), in the presence of a suitable base, such as, for example N,N-diisopropylethylamine, in a suitable solvent system, such as, for example, dichloromethane, at temperatures ranging from 0° C. to boiling point of the respective solvent, preferably the reaction is carried out at room temperature, to furnish alkyl 1H-indazole-3-carboxylate intermediates of general formula (1-2).

Alkyl 1H-indazole-3-carboxylate Intermediates of the general formula (1-2) can be converted to intermediates of general formula (1-4) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-4) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-5).

Intermediates of general formula (1-5) can be converted to intermediates of general formula (1-10) by reaction with a suitably substituted β-ketoester of the general formula (1-9), such as, for example methyl 2-methyl-3-oxobutanoate, in the presence of a suitable base, such as, for example sodium methoxide, in a suitable solvent system, such as, for example, methanol, in a temperature range from room temperature to the boiling point of the respective solvent, preferably the reaction is carried out at 80° C.

Intermediates of general formula (1-10) can be converted to intermediates of general formula (1-11) by reaction with a phosphoric trihalide, such as, for example phosphoric trichloride, in a suitable solvent system, such as, for example, toluene, in a temperature range from room temperature to the boiling point of the respective solvent, preferably the reaction is carried out at 1200.

Intermediates of general formula (1-11) can be reacted with a suitable substituted six membered heterocycle of the general formula (1-12), such as, for example 4-aminopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 1000 to furnish compounds of general formula (I-13). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium (II), dichloro(di-μ-chloro)bis[1,3-bis(2,6-di-iso-propylphenyl) imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:

racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-11) can be reacted with a suitable substituted six membered heterocycle of the general formula (1-12), such as for example 4-aminopyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-13).

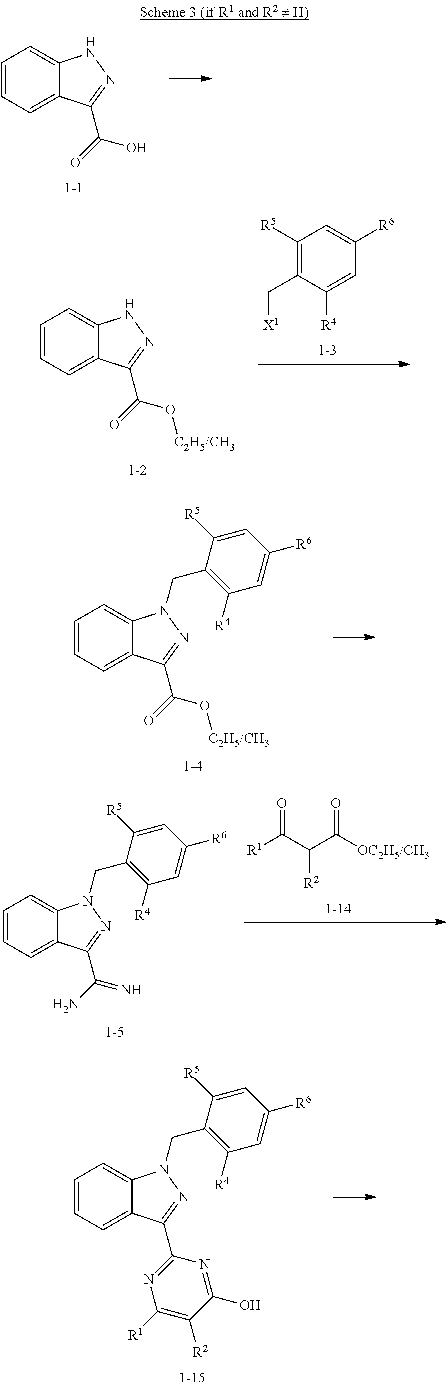

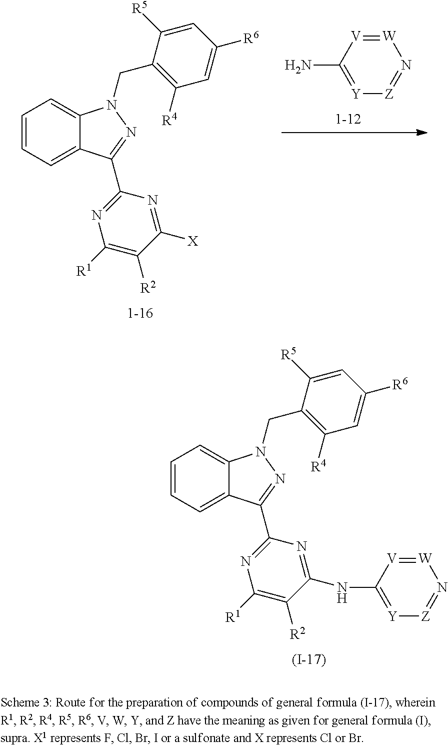

In addition, interconversion of any of the substituents R¹, R², R⁴, R⁵, R⁶, V, W, Y, or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-3, 1-12 and 1-14 are either commercially available or can be prepared according to procedures available from the public domain, as understandable to the person skilled in the art. Specific examples are described in the subsequent paragraphs.

A suitably substituted 1H-indazole-3-carboxylic acid of general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

A suitably substituted 1H-indazole-3-carboxylic acid of general formula (1-1) can be reacted with methanol or ethanol in the presence of peptide coupling agent, such as, for example, HATU (N-[(dimethylamino)(3H-[1,2,3]triazolo[4,5-b]pyridin-3-yloxy)methylidene]-N-methylmethanaminium hexafluorophosphate), in the presence of a suitable base, such as, for example N,N-diisopropylethylamine, in a suitable solvent system, such as, for example, dichloromethane, at temperatures ranging from 0° C. to boiling point of the respective solvent, preferably the reaction is carried out at room temperature, to furnish alkyl 1H-indazole-3-carboxylate intermediates of general formula (1-2).

Alkyl 1H-indazole-3-carboxylate Intermediates of the general formula (1-2) can be converted to intermediates of general formula (1-4) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-4) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-5).

Intermediates of general formula (1-5) can be converted to intermediates of general formula (1-15) by reaction with a suitably substituted β-ketoester of the general formula (1-14), such as, for example methyl methyl 2-fluoro-2-methyl-3-oxobutanoate, in the presence of a suitable base, such as, for example sodium methoxide, in a suitable solvent system, such as, for example, methanol, in a temperature range from room temperature to the boiling point of the respective solvent, preferably the reaction is carried out at 80° C.

Intermediates of general formula (1-15) can be converted to intermediates of general formula (1-16) by reaction with a phosphoric trihalide, such as, for example phosphoric trichloride, in a suitable solvent system, such as, for example, toluene, in a temperature range from room temperature to the boiling point of the respective solvent, preferably the reaction is carried out at 120° C.

Intermediates of general formula (1-16) can be reacted with a suitable substituted six membered heterocycle of the general formula (1-12), such as, for example 4-aminopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-17). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2, 6-diisopropylphenyl) imidazol-2-ylidene](3-chloropyridyl)palladium(II) dichloride, [2-(acetylamino)-4-methoxyphenyl]{1,3-bis[2, 6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium (II), dichloro(di-μ-chloro)bis[1,3-bis(2,6-di-iso-propylphenyl) imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethan-aminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:

racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-16) can be reacted with a suitable substituted six membered heterocycle of the general formula (1-12), such as for example 4-aminopyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-17).

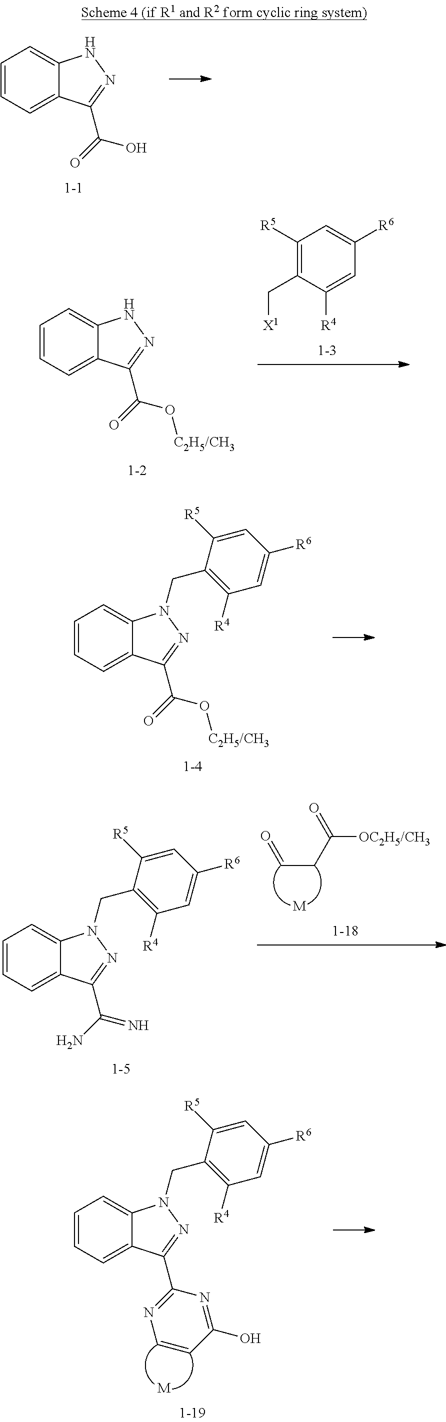

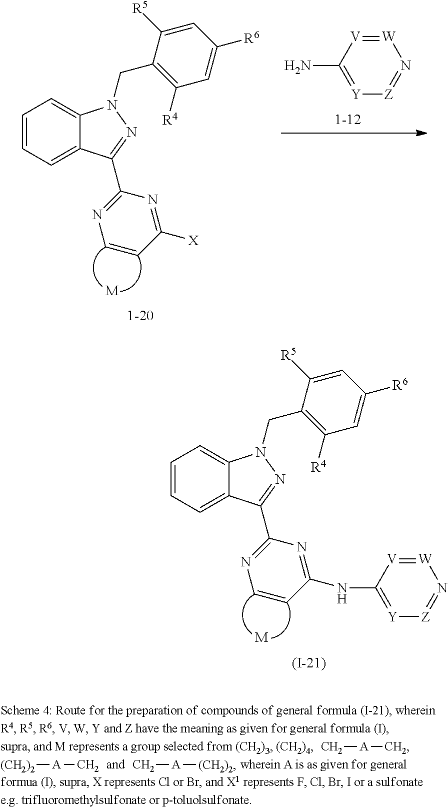

In addition, interconversion of any of the substituents R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-3, 1-12 and 1-18 are either commercially available or can be prepared according to procedures available from the public domain, as understandable to the person skilled in the art. Specific examples are described in the subsequent paragraphs.

A suitably substituted 1H-indazole-3-carboxylic acid of general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

A suitably substituted 1H-indazole-3-carboxylic acid of general formula (1-1) can be reacted with methanol or ethanol in the presence of peptide coupling agent, such as, for example, HATU (N-[(dimethylamino)(3H-[1,2,3]triazolo[4,5-b]pyridin-3-yloxy)methylidene]-N-methylmethanaminium hexafluorophosphate), in the presence of a suitable base, such as, for example N,N-diisopropylethylamine, in a suitable solvent system, such as, for example, dichloromethane, at temperatures ranging from 0° C. to boiling point of the respective solvent, preferably the reaction is carried out at room temperature, to furnish alkyl 1H-indazole-3-carboxylate intermediates of general formula (1-2).

Alkyl 1H-indazole-3-carboxylate Intermediates of the general formula (1-2) can be converted to intermediates of general formula (1-4) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-4) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-5).

Intermediates of general formula (1-5) can be converted to intermediates of general formula (1-19) by reaction with a suitably substituted β-ketoester of the general formula (1-18), such as, for example methyl methyl 2-fluoro-2-methyl-3-oxobutanoate, in the presence of a suitable base, such as, for example sodium methoxide, in a suitable solvent system, such as, for example, methanol, in a temperature range from room temperature to the boiling point of the respective solvent, preferably the reaction is carried out at 80° C.

Intermediates of general formula (1-19) can be converted to intermediates of general formula (1-20) by reaction with a phosphoric trihalide, such as, for example phosphoric trichloride, in a suitable solvent system, such as, for example, toluene, in a temperature range from room temperature to the boiling point of the respective solvent, preferably the reaction is carried out at 120° C.

Intermediates of general formula (1-20) can be reacted with a suitable substituted six membered heterocycle of the general formula (1-12), such as, for example 4-aminopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at at 100° C. to furnish compounds of general formula (I-21). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium (II), dichloro(di-μ-chloro)bis[1,3-bis(2,6-di-iso-propylphenyl) imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:

racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4, 5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-20) can be reacted with a suitable substituted six membered heterocycle of the general formula (1-12), such as for example 4-aminopyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-21).

Compounds of general formula (1-1) can be converted into compounds of general formula (1-2) according to the procedure depicted in Scheme 5.

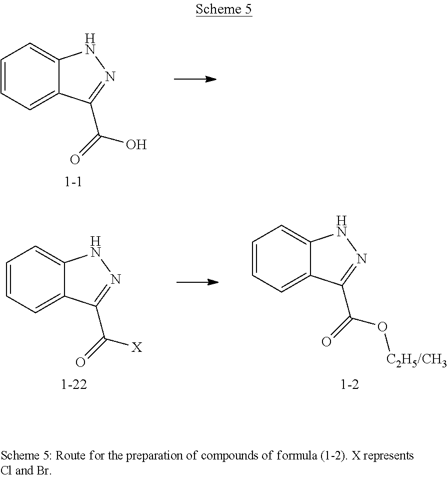

Alternatively substituted 1H-indazole-3-carboxylic acid of general formula (1-1) can be converted to the corresponding substituted 1H-indazole-3-carbonyl halide of general formula (1-22) by treatment with thionyl halides, for example thionyl chloride in a suitable solvent system, such as, for example, toluene, at a temperature between 0° C. and boiling point of the respective solvent, preferably the reaction is carried out at 120° C. The substituted 1H-indazole-3-carbonyl halide can be reacted with methanol or ethanol in the presence of a base, such as, for example, triethylamine, in an suitable solvent system, such as, for example, dichloromethane, at a temperature between −20° C. and boiling point of the respective solvent, preferably the reaction is carried out at 0° C. to yield the desired alkyl 1H-indazole-3-carboxylate intermediates of general formula (1-2).

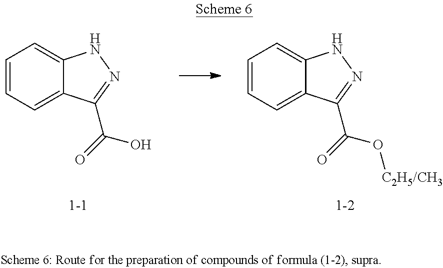

Alternatively compounds of general formula (1-1) are then reacted with methanol or ethanol as mentioned above with a peptide coupling agent, for example N-[(dimethylamino)(3H-[1,2,3]triazolo[4,5-b]pyridin-3-yloxy) methylidene]-N-methylmethanaminium hexafluorophosphate, in a suitable solvent, such as, for example, dichloromethane, in the presence of a suitable base, such as, for example, diisopropylethylamine in a temperature range from −10° C. to the boiling point of the respective solvent, preferably the reaction is carried out at room temperature, to furnish compounds of general formula (1-2).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

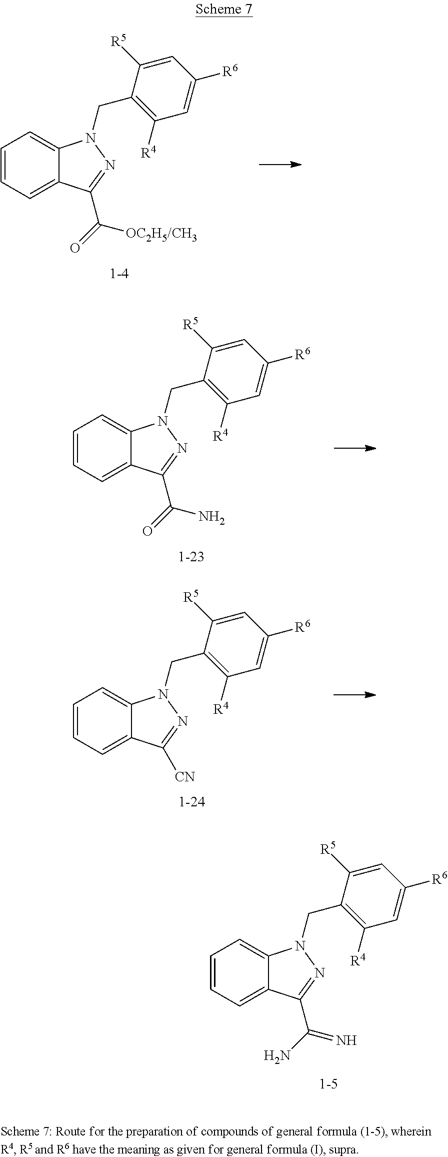

In addition, interconversion of any of the substituent R⁴, R⁵ and R⁶ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Intermediates of general formula (1-4) can be converted to intermediates of general formula (1-23) by reaction with ammonia, in a suitable solvent system, such as, for example, methanol, at a temperature between 0° C. an d boiling point of the respective solvent, preferably the reaction is carried out at 50° C., at a pressure between 1 and 10 bar, preferably the reaction is carried in a sealed vessel.

Intermediates of general formula (1-23) are treated with triflic anhydride, in a suitable solvent system, such as, for example, tetrahydrofuran, in the presence of a suitable base, such as, for example, pyridine, at a temperature between 0° C. and the boiling point of the respective solvent, preferably the reaction is carried out at room temperature, to form the desired intermediate of general formula (1-24).

Intermediates of general formula (1-24) can be converted to intermediates of general formula (1-5) by reaction with a suitable alcoholate, such as, for example sodium methanolate, in a suitable solvent system, such as, for example, the corresponding alcohol, e.g. methanol, at a temperature between room temperature and the boiling point of the respective solvent, preferably the reaction is carried out at room temperature, and subsequent treatment with a suitable source of ammonium, such as for example, ammonium chloride in the presence of a suitable acid, such as for example acetic acid in a temperature range from room temperature to the boiling point of the respective solvent, preferably the reaction is carried out at 50° C.

Compounds of general formula (1-26) can be converted into compounds of general formula (1-6) according to the procedure depicted in Scheme 8a.

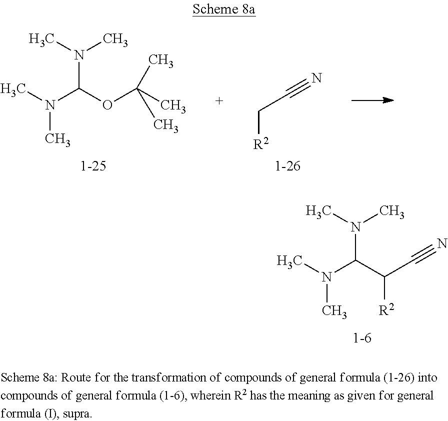

Compounds of general formula (1-25) can be converted into compounds of general formula (1-6) by reaction with a suitable substituted acetonitrile derivative of general formula (1-26), such as, for example methoxyacetonitrile, in a temperature range from room temperature to the boiling point of the respective solvent, preferably the reaction is carried out at 80° C.

Compounds of general formula (1-26) can be converted into compounds of general formula (1-53) according to the procedure depicted in Scheme 8b.

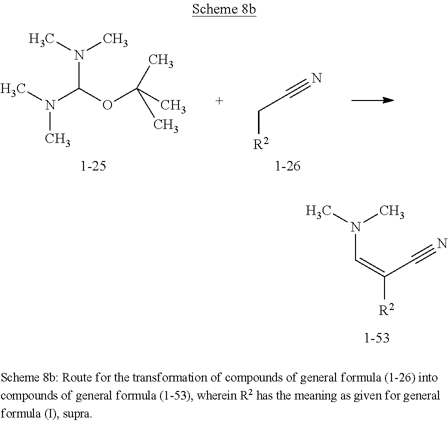

Compounds of general formula (1-26) can be converted into compounds of general formula (1-53 by reaction with a suitable substituted acetonitrile derivative of general formula (1-26), such as, for example tert-butyl 4-(cyanomethyl)piperidine-1-carboxylate, in a suitable solvent system, such as, for example DMF and in a temperature range from room temperature to the boiling point of the respective solvent, preferably the reaction is carried out at 110° C.

Compounds of general formula (1-27) can be converted into compounds of general formula (1-3) according to the procedure depicted in Scheme 9.

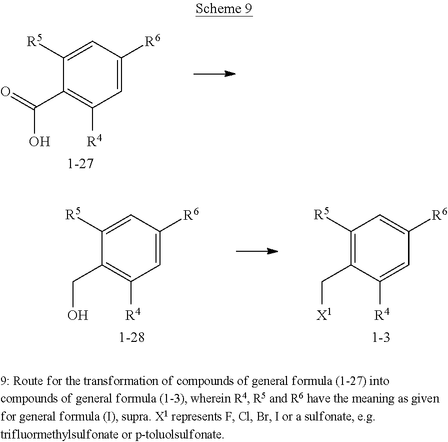

Compounds of general formula (1-27) can be converted into compounds of general formula (1-28) by reaction with a suitable reducing agent, such as, for example borane, in a suitable solvent system, such as, for example, tetrahydrofuran, in a temperature range from −78° C. to boiling point of the respective sol vent, preferably the reaction is carried out at room temperature.

Compounds of general formula (1-28) can be converted into compounds of general formula (1-3) by reaction with a suitable halogenation or sulfonylation agent, such as for example hydrogen bromide, in a suitable solvent, such as, for example, acetic acid, in a temperature range from 0° C. to the boiling point of the respective solvent, preferably the reaction is carried out at room temperature.

Compounds of general formula (1-29) can be converted into compounds of general formula (1-32) according to the procedure depicted in Scheme 10.

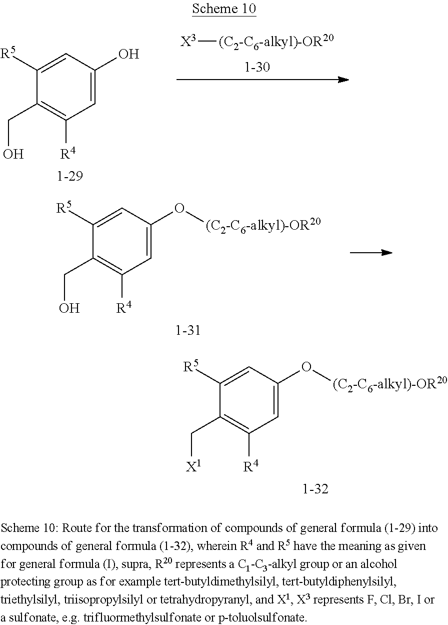

Compounds of general formula (1-29) are then reacted with a compound of general formula (1-30) as mentioned above, in a suitable solvent, such as, for example, DMF, in the presence of a suitable base, such as, for example, sodium hydride in a temperature range from room temperature to the boiling point of the respective solvent, preferably the reaction is carried out at room temperature, to furnish compounds of general formula (1-31).

Compounds of general formula (1-31) can be converted into compounds of general formula (1-32) by reaction with a suitable halogenation or sulfonylation agent, such as for example hydrogen bromide, in a suitable solvent, such as, for example, acetic acid, in a temperature range from 0° C. to the boiling point of the respective solvent, preferably the reaction is carried out at room temperature.

Compounds of general formula (1-8) can be converted into compounds of general formula (1-12) according to the procedure depicted in Scheme 11.

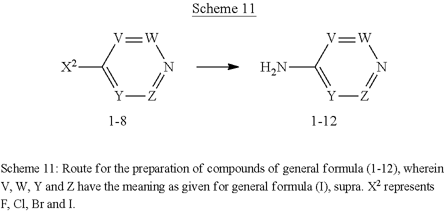

Intermediates of general formula (1-8) can be converted to intermediates of general formula (1-12) by reaction with amine derivative, such as, for example ammonia, in a suitable solvent system, such as, for example, 2-propanol, in a temperature range from room temperature to 120° C., preferably the reaction is carried out at 120° C.

Alternatively, compounds of general formula (1-8) can be converted into compounds of general formula (1-12) according to the procedure depicted in Scheme 12.

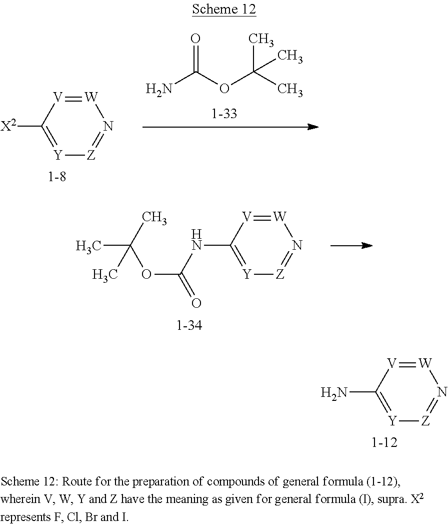

Intermediates of general formula (1-8) can be reacted with a suitable carbamate, such as, for example tert-butyl carbamate (1-33), in the presence of a suitable base, such as, for example caesium carbonate, and a suitable palladium catalyst, such as for example bis(dibenzylideneacetone)-palladium(0), in the presence of a suitable ligand, such as for example 9(9,9-dimethyl-9H-xanthene-4,5-diyl)bis(diphenylphosphine), in a suitable solvent system, such as, for example, 1,4-dioxane, in a temperature range from room temperature to the boiling point of the respective solvent, preferably the reaction is carried out at at 1100 to furnish compounds of general formula (1-34). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl) (chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:

racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4, 5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Intermediates of general formula (1-34) can be converted to intermediates of general formula (1-12) by reaction with Broensted acid, such as, for example hydrogen chloride, in a suitable solvent system, such as, for example, 1,4-dioxane, in a temperature range from room temperature to the boiling point of the respective solvent, preferably the reaction is carried out at 100° C.

A route for the preparation of compounds of general formulae (I-38) and (I-39) is described in Scheme 13.

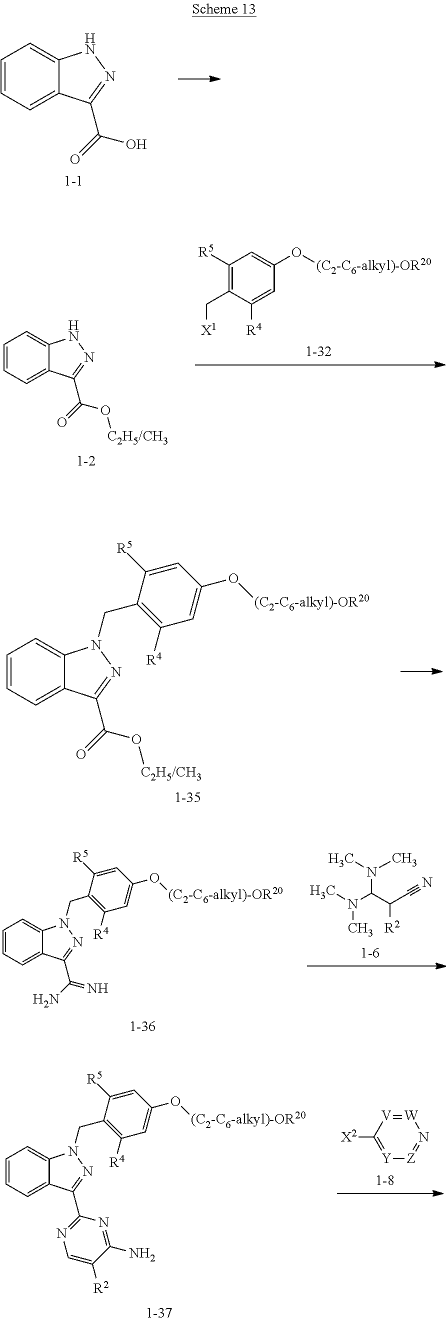

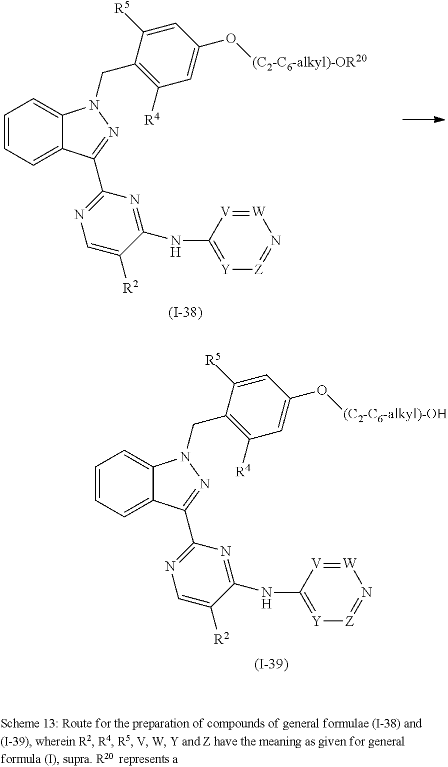

C₁-C₃-alkyl group or an alcohol protecting group as for example tert-butyldimethylsilyl, tert-butyldiphenylsilyl, triethylsilyl, triisopropylsilyl or tetrahydropyranyl, X¹ represents F, Cl, Br, I or a sulfonate, e.g. trifluormethylsulfonate or p-toluolsulfonate, and X² represents F, Cl, Br, I, boronic acid or a boronic acid ester, such as for example 4,4,5,5-tetramethyl-2-phenyl-1,3,2-dioxaborolane (boronic acid pinacole ester).

In addition, interconversion of any of the substituents R², R⁴, R⁵, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

A suitably substituted 1H-indazole-3-carboxylic acid of general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

Alkyl 1H-indazole-3-carboxylate Intermediates of the general formula (1-2) can be converted to intermediates of general formula (1-35) by reaction with a suitable alkylating agent, such as, for example a substituted benzyl halide (1-32), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-35) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-36).

Intermediates of general formula (1-36) can be converted to intermediates of general formula (1-37) by reaction with a suitably substituted 3,3-bis(dimethylamino)propanenitrile of the general formula (1-6, in the presence of a suitable base, such as, for example piperidine, in a suitable solvent system, such as, for example, 3-methylbutan-1-ol, in a temperature range from room temperature to the boiling point of the respective solvent, preferably the reaction is carried out at 100.

Intermediates of general formula (1-37) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at at 100° C. to furnish compounds of general formula (1-38). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium (II), dichloro(di-μ-chloro)bis[1,3-bis(2,6-di-iso-propylphenyl) imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4, 5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-37) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (1-38).

Alternatively intermediates of general formula (1-37) can be reacted with a suitable substituted six membered heterocycle of the general formula (1-8), such as for example 4-fluoropyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish compounds of general formula (1-38).

Compounds of general formula (1-38) are converted to compounds of general formula (I-39) by treatment with a suitable dealkylating agent, such as for example boron trichloride, in a suitable solvent, such as, for example, dichloromethane, in a temperature range from room temperature to the boiling point of the respective solvent, preferably the reaction is carried out at 40° C.

A route for the preparation of compounds of general formulae (I-43) and (I-44) is described in Scheme 14.

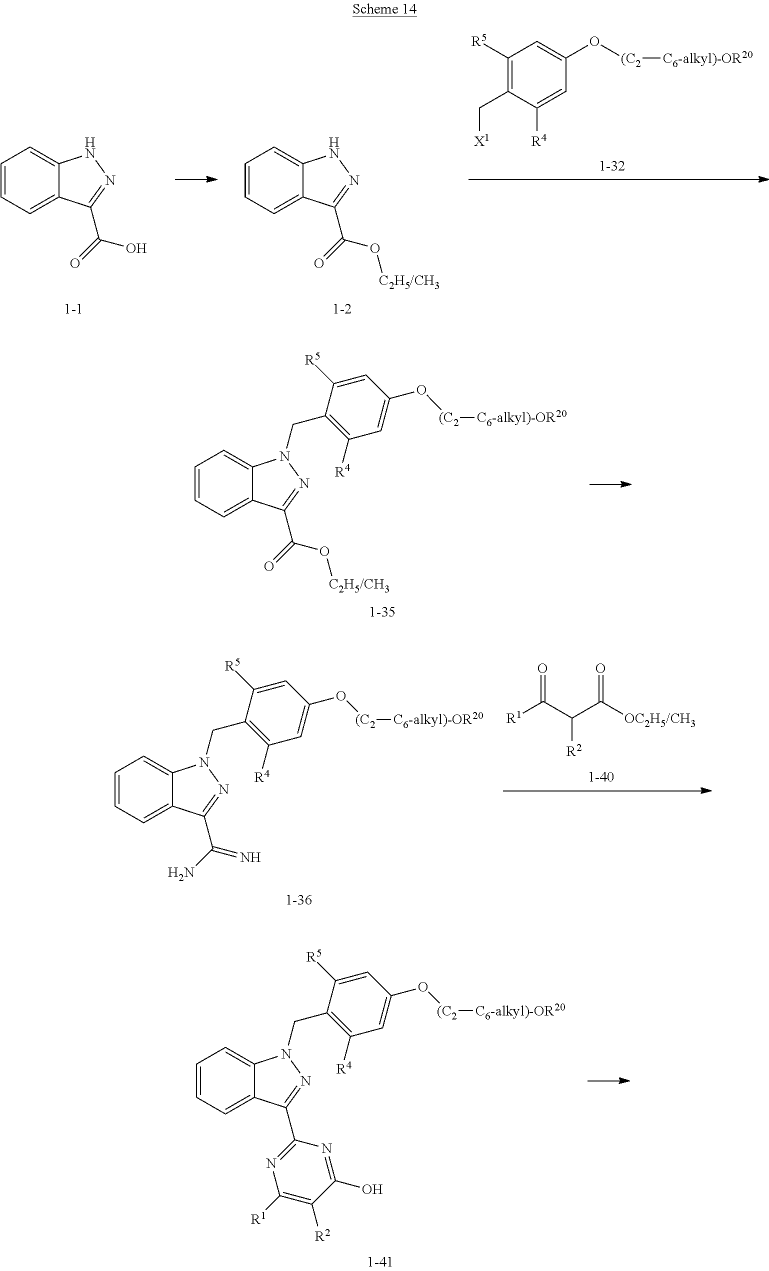

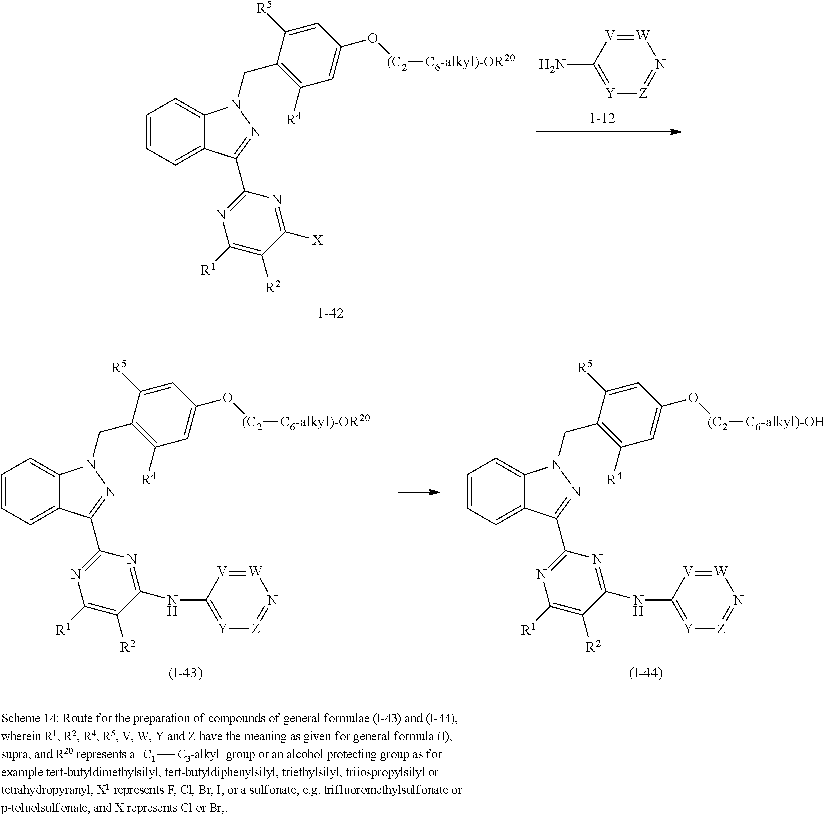

In addition, interconversion of any of the substituents R¹, R², R⁴, R⁵, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

A suitably substituted 1H-indazole-3-carboxylic acid of general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

Alkyl 1H-indazole-3-carboxylate Intermediates of the general formula (1-2) can be converted to intermediates of general formula (1-35) by reaction with a suitable alkylating agent, such as, for example a substituted benzyl halide (1-32), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-35) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-36).

Intermediates of general formula (1-36) can be converted to intermediates of general formula (1-41) by reaction with a suitably substituted β-ketoester of the general formula (1-40), such as, for example methyl methyl 2-fluoro-2-methyl-3-oxobutanoate, in the presence of a suitable base, such as, for example sodium methoxide, in a suitable solvent system, such as, for example, methanol, in a temperature range from room temperature to the boiling point of the respective solvent, preferably the reaction is carried out at 80° C.

Intermediates of general formula (1-41) can be converted to intermediates of general formula (1-42) by reaction with a phosphoric trihalide, such as, for example phosphoric trichloride, in a suitable solvent system, such as, for example, toluene, in a temperature range from room temperature to the boiling point of the respective solvent, preferably the reaction is carried out at 120° C.

Intermediates of general formula (1-42) can be reacted with a suitable substituted six membered heterocycle of the general formula (1-12), such as, for example 4-aminopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at at 100° C. to furnish compounds of general formula (1-43). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium(II), dichloro(di-μ-chloro)bis[1,3-bis(2,6-di-iso-propylphenyl) imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2,6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-42) can be reacted with a suitable substituted six membered heterocycle of the general formula (1-12), such as for example 4-aminopyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish compounds of general formula (1-43).

Compounds of general formula (1-43) are converted to compounds of general formula (I-44) by treatment with a suitable dealkylating agent, such as for example boron trichloride, in a suitable solvent, such as, for example, dichloromethane, in a temperature range from room temperature to the boiling point of the respective solvent, preferably the reaction is carried out at 40° C.

An alternative route for the preparation of compounds of general formula (I-44) is described in Scheme 15.

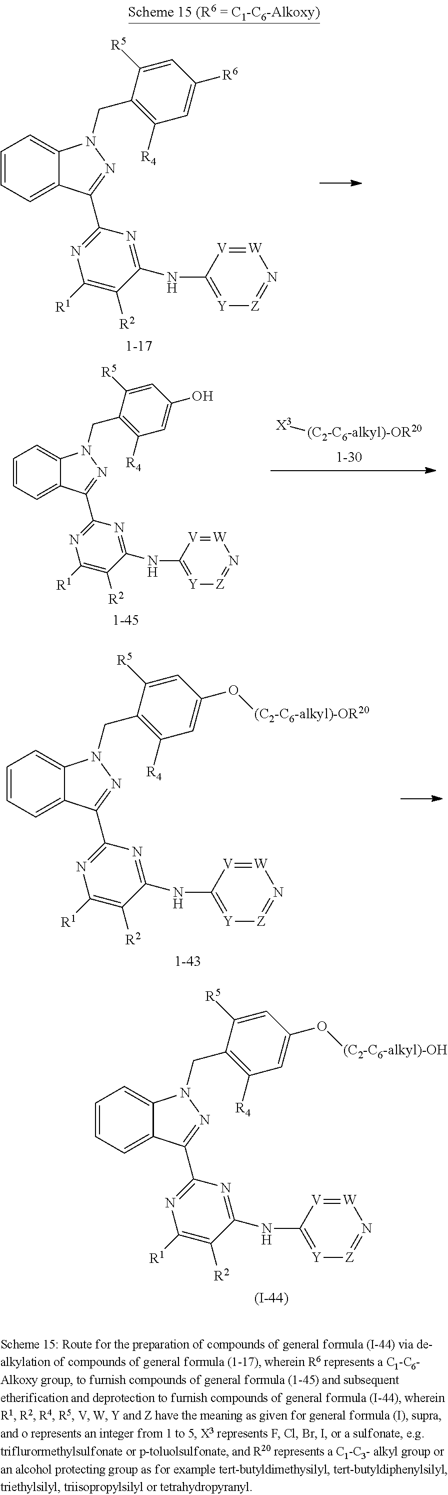

In addition, interconversion of any of the substituents R¹, R², R⁴, R⁵, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds of general formula (1-30), wherein X¹ represents leaving group such as for example a Cl, Br or I, or X¹ stands for an aryl sulfonate such as for example p-toluene sulfonate, or for an alkyl sulfonate such as for example methane sulfonate or trifluoromethane sulfonate (triflate group), are either commercially available or can be prepared according to procedures available from the public domain, as understandable to the person skilled in the art. Specific examples are described in the subsequent paragraphs.

Compounds of general formula (1-17) are converted to compounds of general formula (1-45) by treatment with a suitable demethylating agent, such as for example boron trichloride, in a suitable solvent, such as, for example, dichloromethane, in a temperature range from room temperature to the boiling point of the respective solvent, preferably the reaction is carried out at 40° C.

Compounds of general formula (1-45) are then reacted with a compound of general formula (1-30) as mentioned above, in a suitable solvent, such as, for example, DMF, in the presence of a suitable base, such as, for example, potassium carbonate in a temperature range from room temperature to the boiling point of the respective solvent, preferably the reaction is carried out at room temperature, to furnish compounds of general formula (1-43).

Compounds of general formula (1-43) are then reacted with a suitable Broensted acid, such as, for example, hydrogen chloride, in a suitable solvent, such as, for example, dioxane, in a temperature range from room temperature to the boiling point of the respective solvent, preferably the reaction is carried out at room temperature, to furnish compounds of general formula (I-44).

Compounds of general formula (I) can alternatively be synthesised from compounds of general formula (I-45), via debenzylation and subsequent benzylation according to the procedure depicted in Scheme 16.

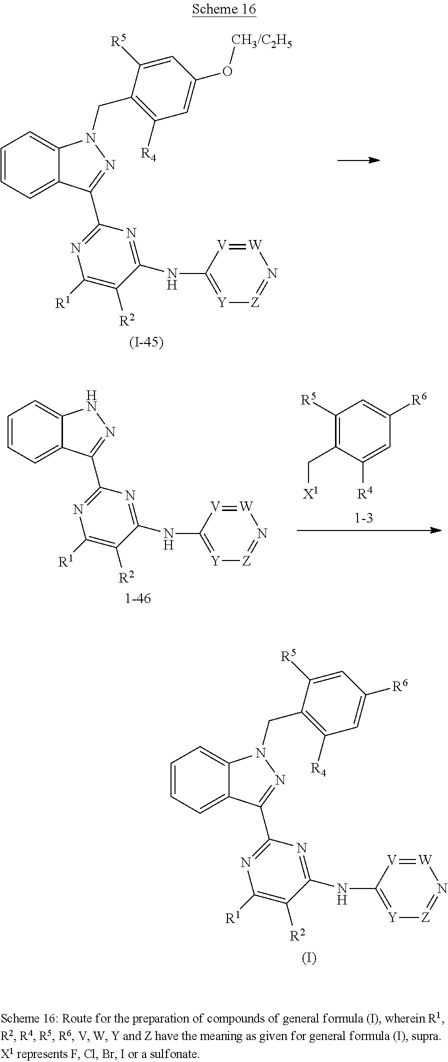

In addition, interconversion of any of the substituents R¹, R², R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds of general formula (1-45) are converted to intermediates of general formula (1-46) by treatment with a suitable acid system, such as, for example a mixture of trifluoroacetic acid and trifluoromethanesulfonic acid, in a suitable solvent, such as, for example, dichloroethan, in a temperature range from room temperature to the boiling point of the respective solvent, preferably the reaction is carried out at room temperature.

Intermediates of general formula (1-46) can be reacted with a suitably substituted benzyl halide or benzyl sulfonate of general formula (1-3), such as, for example, a benzyl bromide, in a suitable solvent system, such as, for example, tetrahydrofuran, in the presence of a suitable base, such as, for example, sodium hydride in a temperature range from room temperature to the boiling point of the respective solvent, preferably the reaction is carried out at room temperature, to furnish compounds of general formula (I).

Compounds of general formula (I-44) can be converted into compounds of general formula (I-52) according to the procedure depicted in Scheme 17.

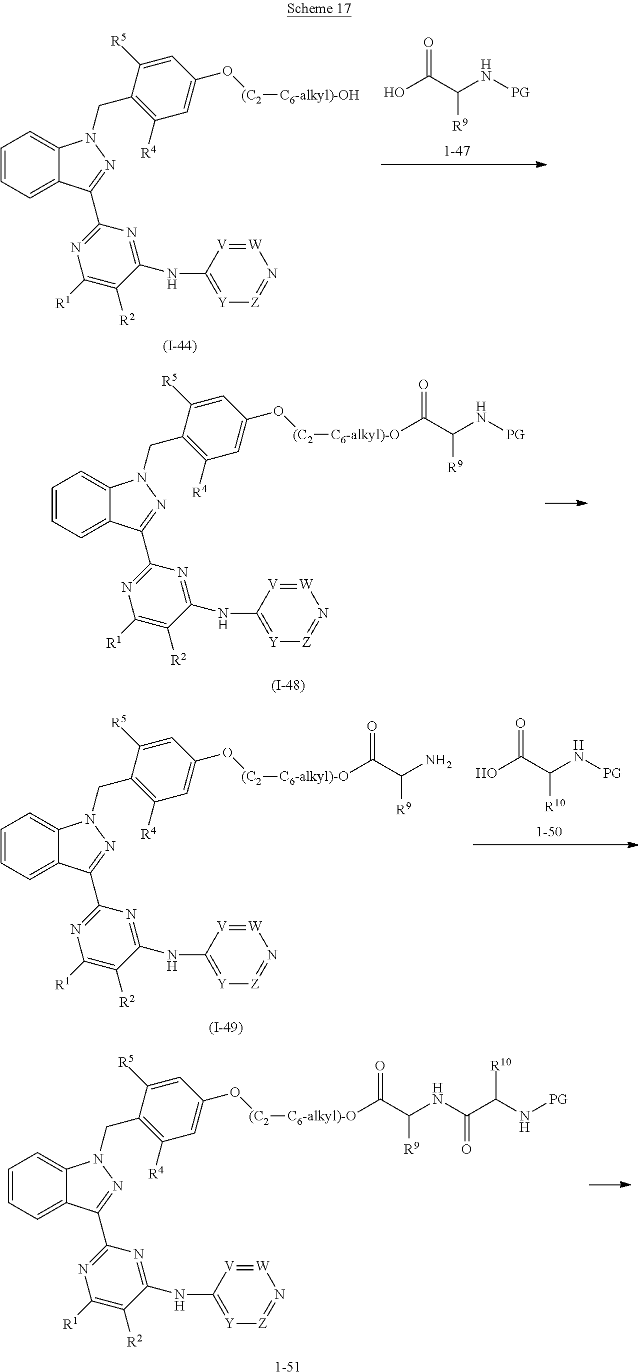

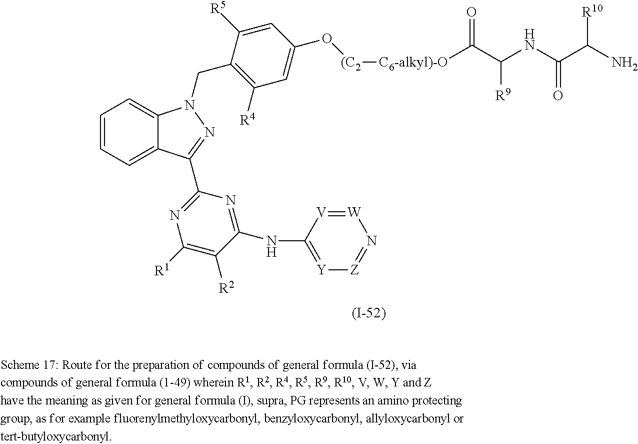

In addition, interconversion of any of the substituents R¹, R², R⁴, R⁵, R⁹, R¹⁰, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds of general formula (I-44) are reacted with a compound of general formula (1-47) with a peptide coupling agent, for example N-[(dimethylamino)(3H-[1,2,3]triazolo[4,5-b]pyridin-3-yloxy)methylidene]-N-methylmethanaminium hexafluorophosphate, in a suitable solvent, such as, for example, dichloromethane, in the presence of a suitable base, such as, for example, diisopropylethylamine in a temperature range from −10° C. to the boiling point of the respective solvent, preferably the reaction is carried out at room temperature, to furnish compounds of general formula (1-48).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

Intermediates of general formula (1-48) can be converted to intermediates of general formula (1-49) by reaction with Broensted acid, such as, for example trifluoroacetic acid, in a suitable solvent system, such as, for example, dichloromethane, in a temperature range from room temperature to the boiling point of the respective solvent, preferably the reaction is carried out at room temperature.

Compounds of general formula (1-49) are then reacted with a compound of general formula (1-50) as mentioned above with a peptide coupling agent, for example N-[(dimethylamino)(3H-[1,2,3]triazolo[4,5-b]pyridin-3-yloxy)methylidene]-N-methylmethanaminium hexafluorophosphate, in a suitable solvent, such as, for example, dichloromethane, in the presence of a suitable base, such as, for example, diisopropylethylamine in a temperature range from −10° C. to the boiling point of the respective solvent, preferably the reaction is carried out at room temperature, to furnish compounds of general formula (1-51).

Appropriate peptide synthesis methods and their applications are well-known to the person skilled in the art (see for example N. Leo Benoitin in Chemistry of Peptide Synthesis, CRC Press 2005; John Jones in Amino Acids and Peptide Synthesis, Oxford University Press, 2002 and Norbert Sewald and Hans-Dieter Jakubke in Peptides: Chemistry and Biology, Wiley-VCH, 2009).

Intermediates of general formula (1-51) can be converted to compounds of general formula (I-52) by reaction with Broensted acid, such as, for example trifluoroacetic acid, in a suitable solvent system, such as, for example, dichloromethane, in a temperature range from room temperature to the boiling point of the respective solvent, preferably the reaction is carried out at room temperature.

Compounds of general formula (1-5) can be converted into compounds of general formula (Ia) according to the procedure depicted in Scheme 18.

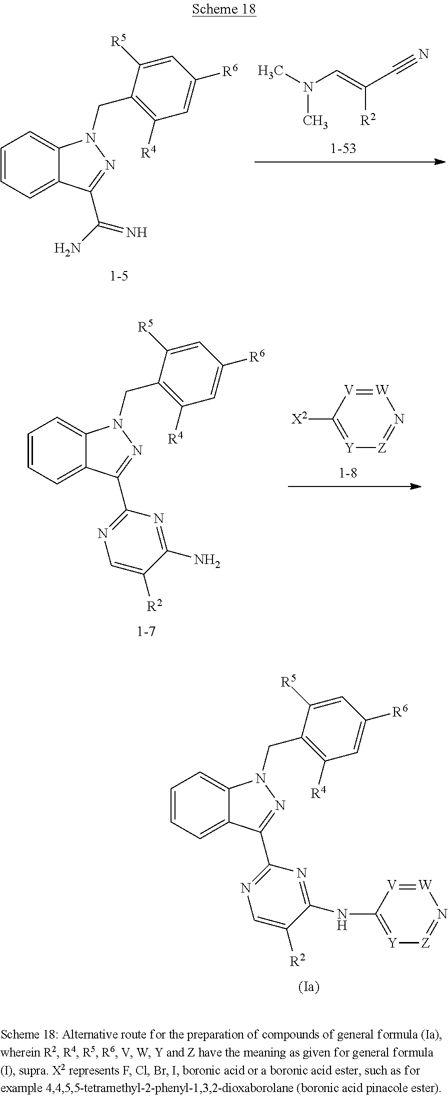

In addition, interconversion of any of the substituents R², R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-7) by reaction with a suitably substituted (dimethylamino)methylene alkanenitrile derivative of the general formula (1-53), in the presence of a suitable base, such as, for example 1,8-diazabicyclo(5.4.0)undec-7-en, in a suitable solvent, such as, for example, pyridine, in a temperature range from room temperature to the boiling point of the respective solvent, preferably the reaction is carried out at 110° C.

Intermediates of general formula (1-7) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (Ia). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (Ia).

Alternatively intermediates of general formula (1-7) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (Ia).

An alternative route for the conversion of compounds of general formula (1-5) into compounds of general formula (Ia) is depicted in Scheme 19.

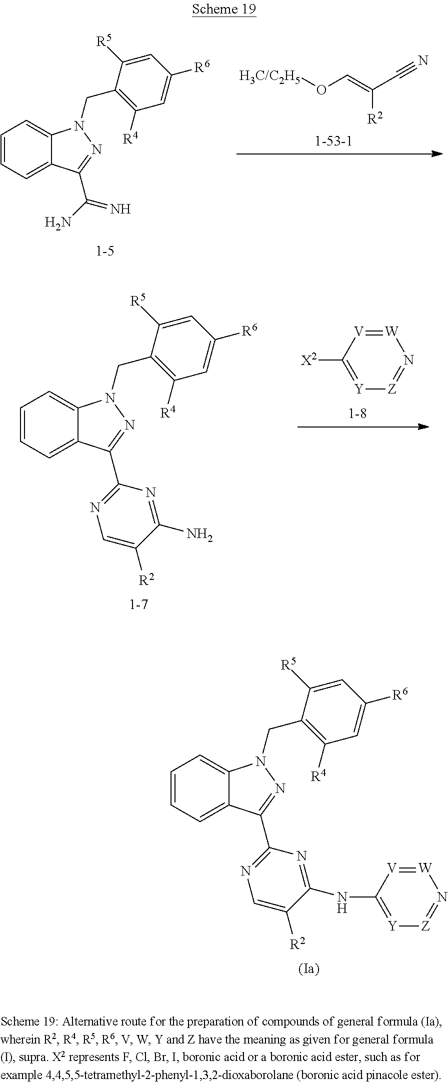

In addition, interconversion of any of the substituents R², R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-7) by reaction with a suitably substituted 3-methoxyacrylonitrile of the general formula (1-53-1), in the presence of a suitable base, such as, for example sodium methanolate, in a suitable solvent such as, for example, methanol, in a temperature range from room temperature to the boiling point of the respective solvent, preferably the reaction is carried out at 65° C.

Intermediates of general formula (1-7) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (Ia). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium(II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (Ia).

Alternatively intermediates of general formula (1-7) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (la).

Compounds of general formula (1-5) can be converted into compounds of general formula (1-54) according to the procedure depicted in Scheme 20.

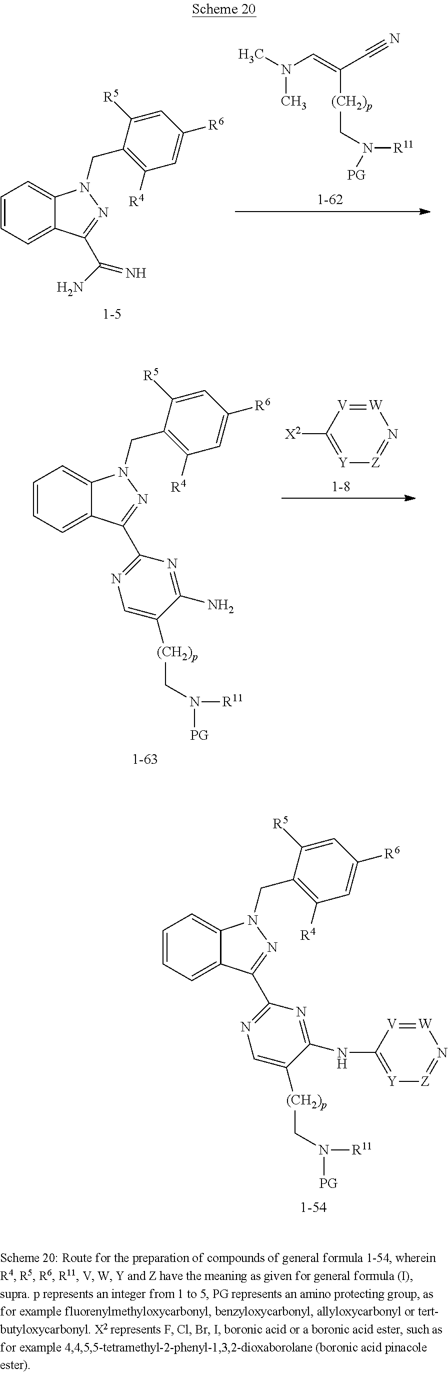

In addition, interconversion of any of the substituents R⁴, R⁵, R⁶, R¹¹, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents.

Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-63) by reaction with a suitably substituted (dimethylamino)methylene alkanenitrile derivative of the general formula (1-62), in the presence of a suitable base, such as, for example 1,8-diazabicyclo(5.4.0)undec-7-en, in a suitable solvent, such as, for example, pyridine, in a temperature range from room temperature to the boiling point of the respective solvent, preferably the reaction is carried out at 110° C.

Intermediates of general formula (1-63) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (1-54). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′4,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-63) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (1-54).

Alternatively intermediates of general formula (1-63) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (1-54).

An alternative route for the conversion of compounds of general formula (1-5) into compounds of general formula (1-54) is depicted in Scheme 21.

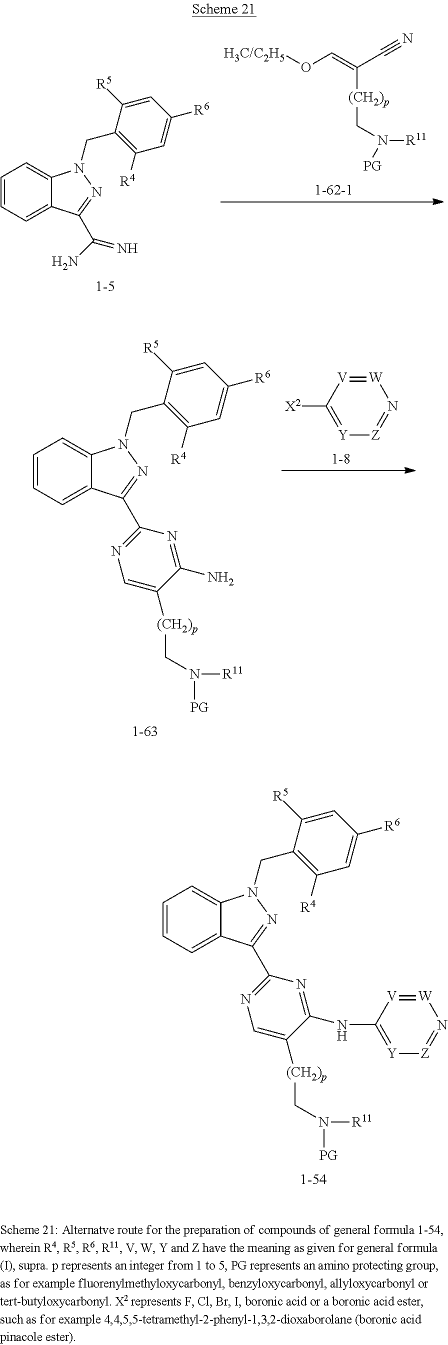

In addition, interconversion of any of the substituents R⁴, R⁵, R⁶, R¹¹, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-63) by reaction with a suitably substituted 3-methoxyacrylonitrile of the general formula (1-62-1), in the presence of a suitable base, such as, for example sodium methanolate, in a suitable solvent, such as, for example, methanol, in a temperature range from room temperature to the boiling point of the respective solvent, preferably the reaction is carried out at 65° C.

Intermediates of general formula (1-63) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (1-54). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl) (chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-63) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (1-54).

Alternatively intermediates of general formula (1-63) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (1-54).

One route for the preparation of compounds of general formulae (I-55) and (I-57) is described in Scheme 22.

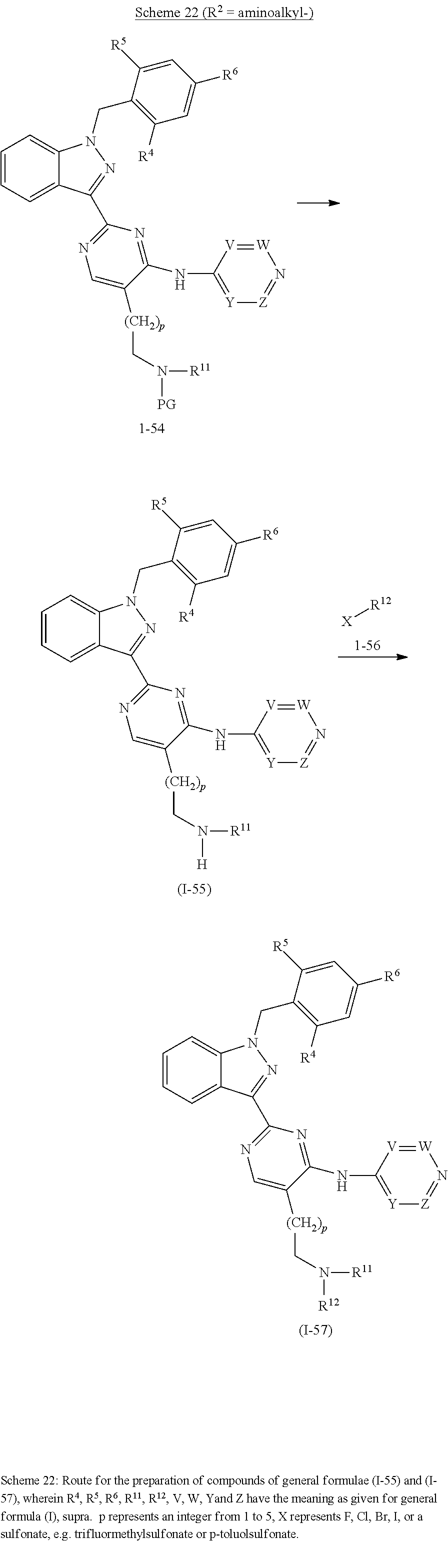

In addition, interconversion of any of the substituents R⁴, R⁵, R⁶, R¹¹, R¹², V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-56 are either commercially available or can be prepared according to procedures available from the public domain, as understandable to the person skilled in the art. Specific examples are described in the subsequent paragraphs.

Intermediates of general formula (1-54) can be converted to intermediates of general formula (I-55) by reaction with Broensted acid, such as, for example trifluoroacetic acid, in a suitable solvent system, such as, for example, dichloromethane, in a temperature range from room temperature to the boiling point of the respective solvent, preferably the reaction is carried out at room temperature.

Intermediates of general formula (I-55) can be converted to intermediates of general formula (I-57) by reaction with a sulfonate of general formula (1-56), such as, for example 2,2-difluoroethyl trifluoromethanesulfonate, in the presence of a suitable base, such as, for example potassium carbonate, in a suitable solvent system, such as, for example, dichloromethane, in a temperature range from room temperature to the boiling point of the respective solvent, preferably the reaction is carried out at room temperature.

An alternative route for the preparation of compounds of general formula (I-57) is described in Scheme 23.

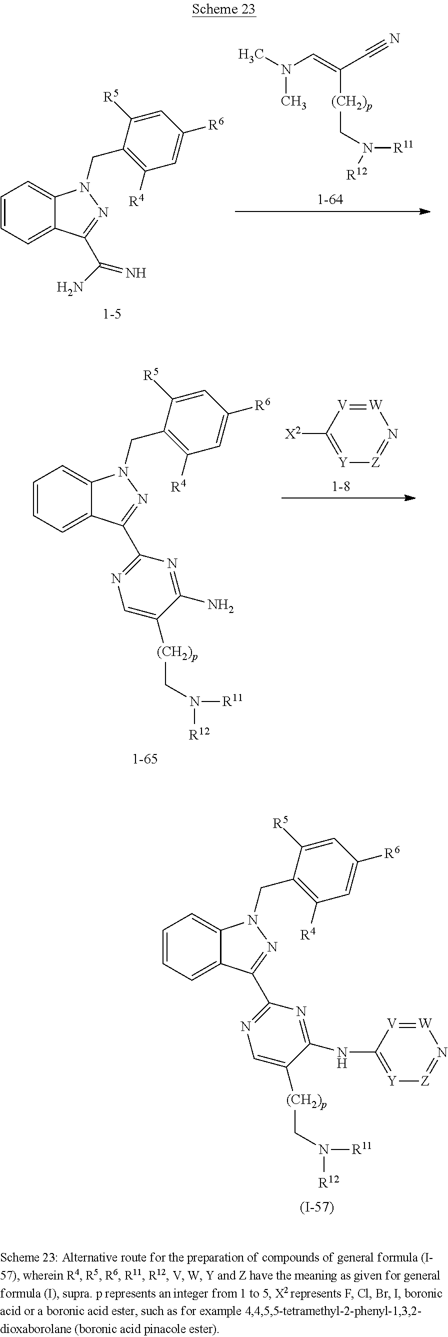

In addition, interconversion of any of the substituents R⁴, R⁵, R⁶, R¹¹, R¹², V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-65) by reaction with a suitably substituted (dimethylamino)methylene alkanenitrile derivative of the general formula (1-64), in the presence of a suitable base, such as, for example 1,8-diazabicyclo(5.4.0)undec-7-en, in a suitable solvent, such as, for example, pyridine, in a temperature range from room temperature to the boiling point of the respective solvent, preferably the reaction is carried out at 110° C.

Intermediates of general formula (1-65) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-57). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′4,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4, 5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-65) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (I-57).

Alternatively intermediates of general formula (1-65) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-57).

An alternative route for the preparation of compounds of general formula (I-57) is described in Scheme 24.

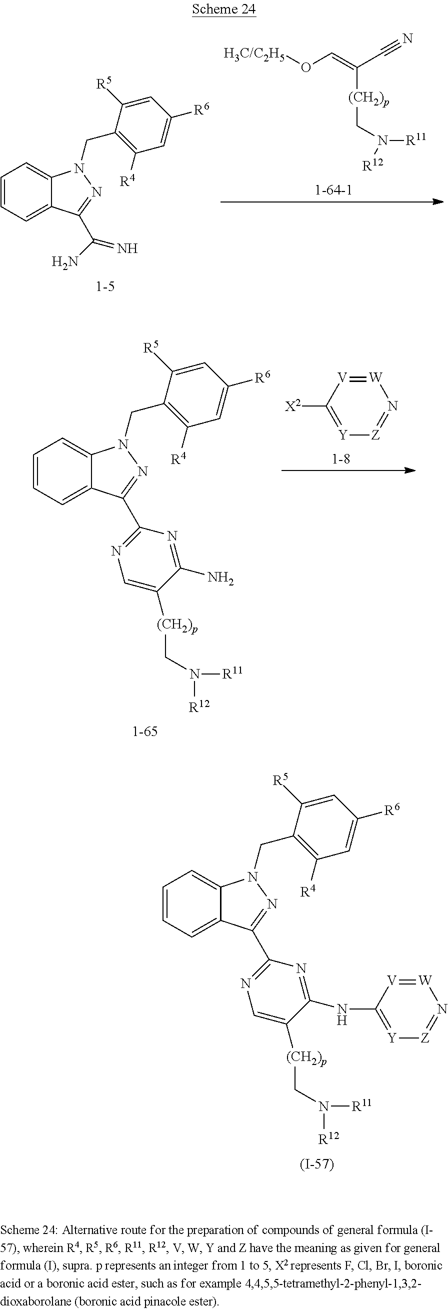

In addition, interconversion of any of the substituents R⁴, R⁵, R⁶, R¹¹, R¹², V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-65) by reaction with a suitably substituted 3-methoxyacrylonitrile of the general formula (1-64-1), in the presence of a suitable base, such as, for example sodium methanolate, in a suitable solvent, such as, for example, methanol, in a temperature range from room temperature to the boiling point of the respective solvent, preferably the reaction is carried out at 65° C.

Intermediates of general formula (1-65) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-57). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl) (chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-65) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (I-57).

Alternatively intermediates of general formula (1-65) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-57).

A route for the preparation of compounds of general formula (I-58) is described in Scheme 25.

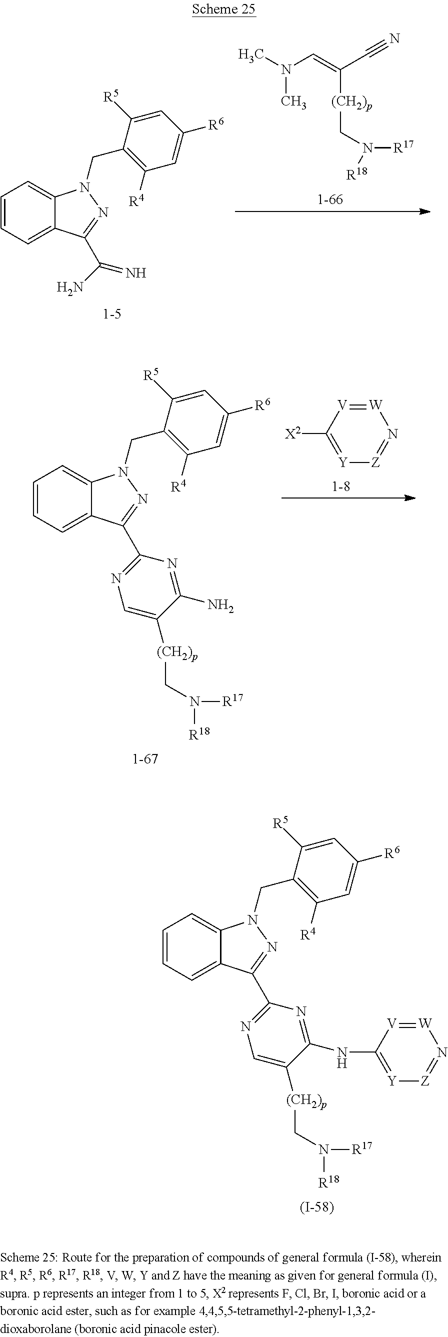

In addition, interconversion of any of the substituents R⁴, R⁵, R⁶, R¹¹, R¹², V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-67) by reaction with a suitably substituted (dimethylamino)methylene alkanenitrile derivative of the general formula (1-66), in the presence of a suitable base, such as, for example 1,8-diazabicyclo(5.4.0)undec-7-en, in a suitable solvent, such as, for example, pyridine, in a temperature range from room temperature to the boiling point of the respective solvent, preferably the reaction is carried out at 110° C.

Intermediates of general formula (1-67) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-58). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′4,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4, 5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-67) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (I-58).

Alternatively intermediates of general formula (1-67) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-58).

An alternative route for the preparation of compounds of general formula (I-58) is described in Scheme 26.

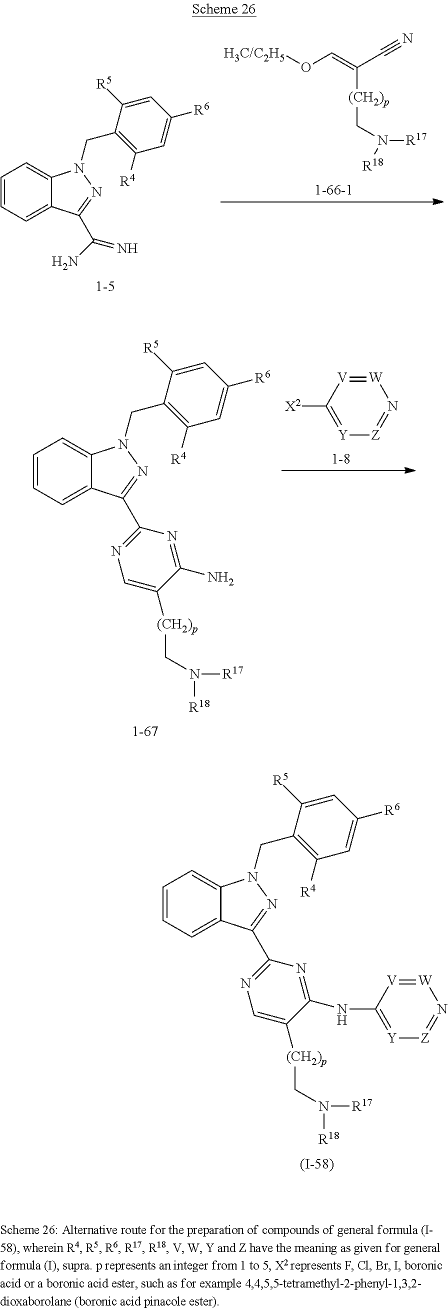

In addition, interconversion of any of the substituents R⁴, R⁵, R⁶, R¹¹, R¹², V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-67) by reaction with a suitably substituted 3-methoxyacrylonitrile of the general formula (1-66-1), in the presence of a suitable base, such as, for example sodium methanolate, in a suitable solvent, such as, for example, methanol, in a temperature range from room temperature to the boiling point of the respective solvent, preferably the reaction is carried out at 65° C.

Intermediates of general formula (1-67) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-58). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl) (chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-67) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (I-58).

Alternatively intermediates of general formula (1-67) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-58).

Compounds of general formula (1-5) can be converted into compounds of general formula (I-59) according to the procedure depicted in Scheme 27.

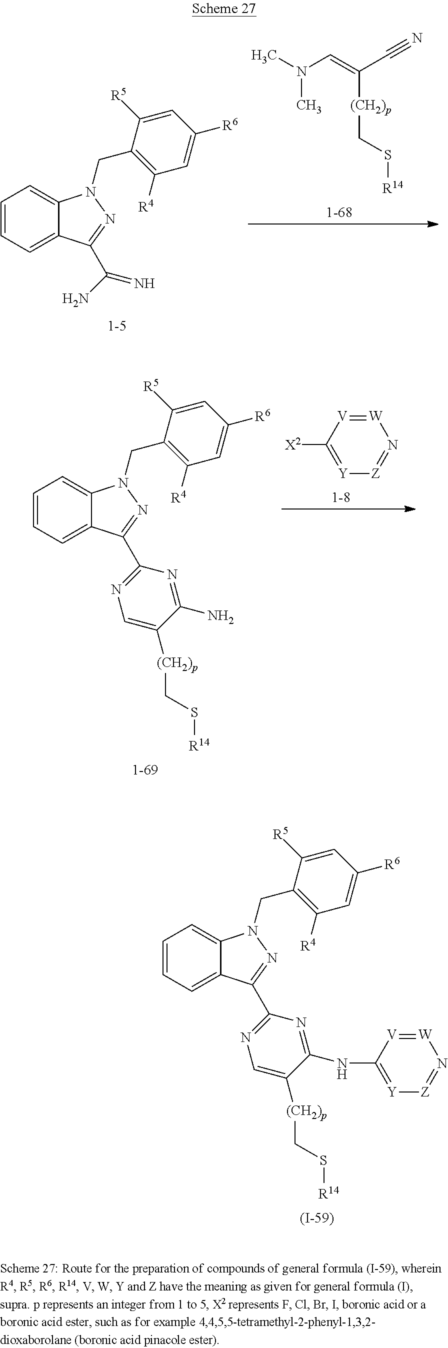

In addition, interconversion of any of the substituents R⁴, R⁵, R⁶, R¹⁴, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-69) by reaction with a suitably substituted (dimethylamino)methylene alkanenitrile derivative of the general formula (1-68), in the presence of a suitable base, such as, for example 1,8-diazabicyclo(5.4.0)undec-7-en, in a suitable solvent, such as, for example, pyridine, in a temperature range from room temperature to the boiling point of the respective solvent, preferably the reaction is carried out at 110° C.

Intermediates of general formula (1-69) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-59). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium (II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′4,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl)(chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium-dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4, 5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-69) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (I-59).

Alternatively intermediates of general formula (1-69) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-59).

An alternative route for the preparation of compounds of general formula (I-59) is described in Scheme 28.

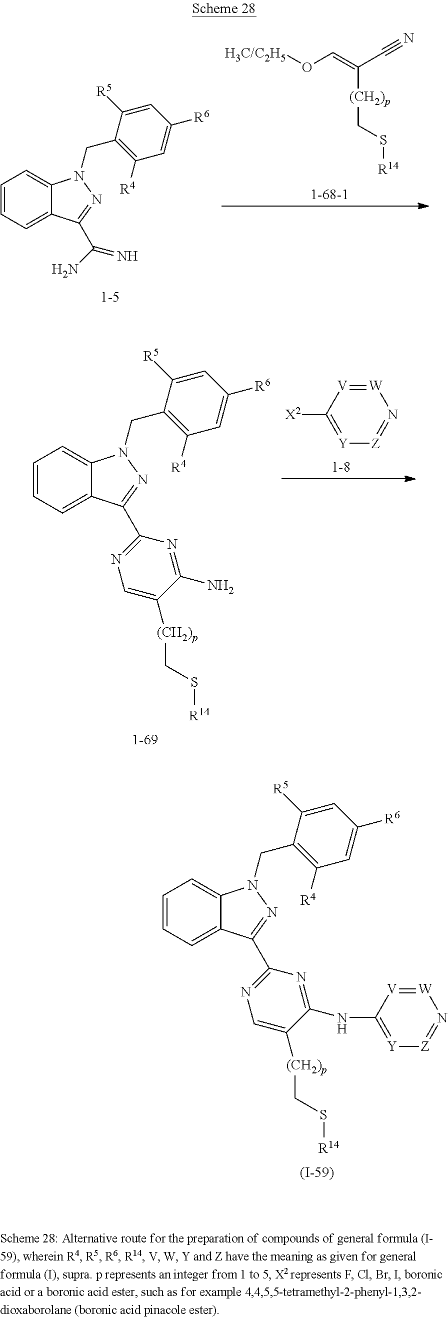

In addition, interconversion of any of the substituents R⁴, R⁵, R⁶, R¹⁴, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent para-graphs.

Compound 1-8 is either commercially available or can be prepared according to procedures available from the public domain, as understandable to the person skilled in the art as referred to below.

Intermediates of general formula (1-5) can be converted to intermediates of general formula (1-69) by reaction with a suitably substituted 3-methoxyacrylonitrile of the general formula (1-68-1), in the presence of a suitable base, such as, for example sodium methanolate, in a suitable solvent system, such as, for example, methanol, in a temperature range from room temperature to the boiling point of the respective solvent, preferably the reaction is carried out at 65° C.

Intermediates of general formula (1-69) can be reacted with a suitable substituted six membered heterocycle of the general formula (1-8), such as, for example 4-bromopyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at 100° C. to furnish compounds of general formula (I-59). Alternatively the following palladium catalysts can be used:

allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium (II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl)palladium (II), dichloro(di-μ-chloro)bis[1,3-bis(2,6-di-iso-propylphenyl)imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium (II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl[2′,4′,6′-tri(propan-2-yl)biphenyl-2-yl]-lambda5-phosphanyl}palladium, (2′-aminobiphenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl)phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methanesulfonato-kappaO)palladium-[2′,6′-bis(propan-2-yloxy)biphenyl-2-yl](dicyclohexyl)phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl) (chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:
racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine.

Alternatively intermediates of general formula (1-69) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (I-59).

Alternatively intermediates of general formula (1-69) can be reacted with a suitable substituted six membered heterocycle of the general formula (1-8), such as for example 4-fluoropyridine, in the presence of a suitable base, such as, for example sodium hydride, in a suitable solvent system, such as, for example, DMF, in a temperature range from room temperature to the boiling point of the respective solvent, preferably the reaction is carried out at 90° C. to furnish compounds of general formula (I-59).

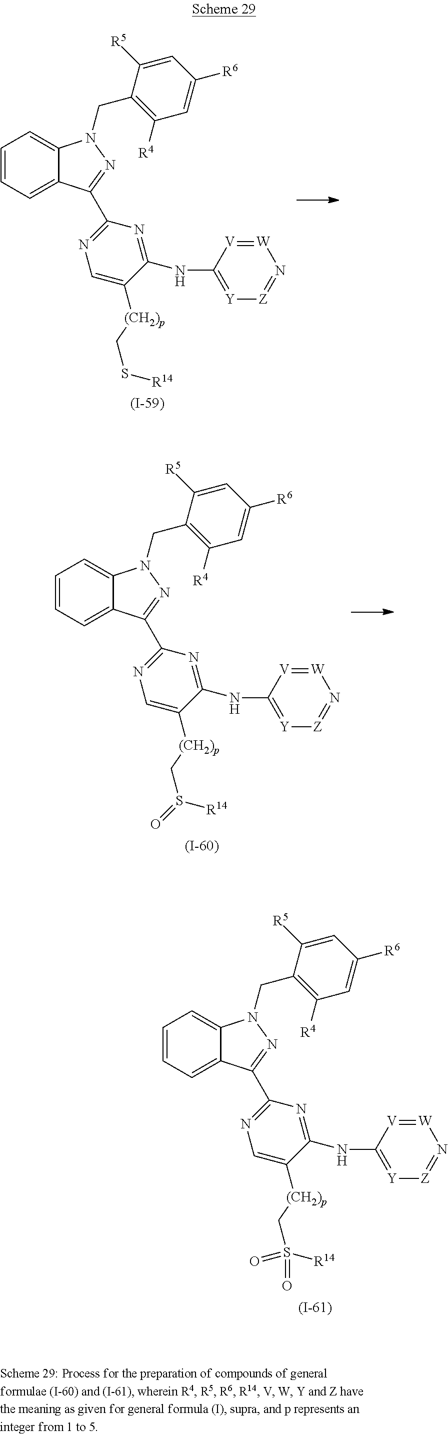

In addition, interconversion of any of the substituents R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds of general formula (I-59) are converted to compounds of general formula (I-60) by treatment with a suitable oxidation agent, such as for example meta-chloroperbenzoic acid, in a suitable solvent, such as, for example, chloroform, in a temperature range from 0° C. to the boiling point of the respective solvent, preferably the reaction is carried out at 0° C.

Compounds of general formula (I-60) can be converted into compounds of general formula (I-61) by treatment with a suitable oxidation agent, such as for example hydrogen peroxide and the reagent diethyl azodicarboxylate, in a suitable solvent, such as, for example, tetrahydrofuran, in a temperature range from 0° C. to the boiling point of the respective solvent, preferably the reaction is carried out at 50° C.

Compounds of general formulae (I-62) and (I-63) can be synthesized from compounds of general formula (I-60) according to the procedure depicted in Scheme 30.

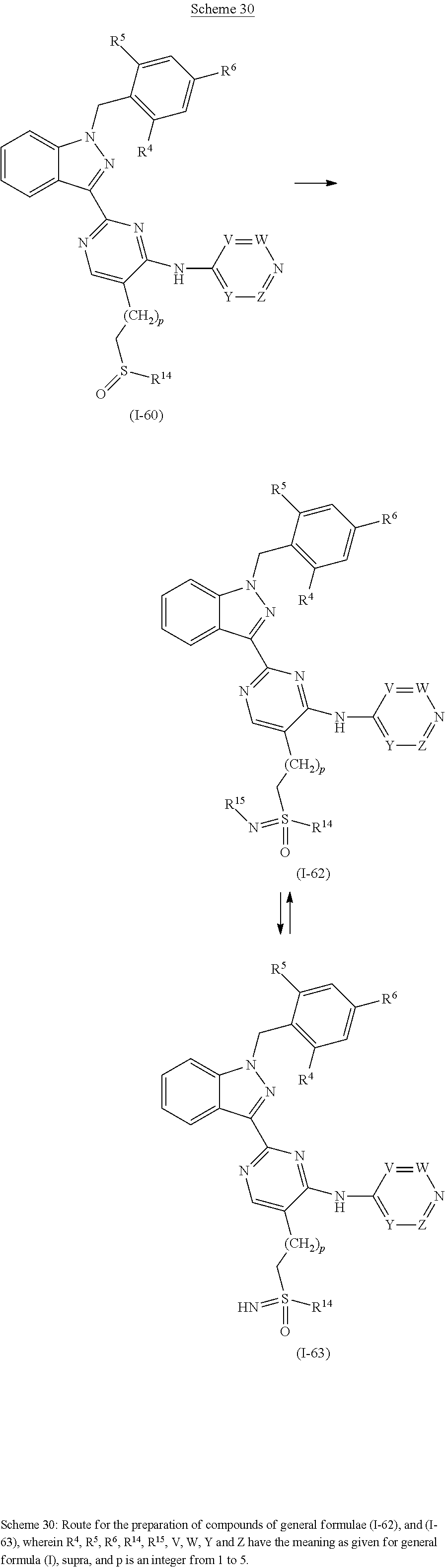

In addition, interconversion of any of the substituents R⁴, R⁵, R⁶, R¹⁵, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds of general formula (I-60) can be reacted to the protected sulfoximines with a suitable reagent mixture, such as, for example 2,2,2-trifluoro acetamide, iodo-benzene diacetate and magnesium oxide, with a suitable catalyst, such as, for example, rhodium(II) acetate dimer, in a suitable solvent system, such as, for example, DCM, in a temperature range from 0° C. to the boiling point of the respective solvent, preferably the reaction is carried out at room temperature to furnish the protected compounds. Deprotection can be accomplished under suitable conditions, such as, for example in the case of trifluoroacetate, a suitable base, such as, for example, potassium carbonate, in a suitable solvent system, such as, for example, methanol, in a temperature range form 0° C. to the boiling point of the respective solvent, preferably the reaction is carried out at room temperature to furnish the compounds of general formula (I-63). The sulfoximines of general formula (1-63) can be N-functionalized by several methods to furnish sulfoximines of general formula (I-62).

For the preparation of N-functionalized sulfoximines multiple methods are known:
Alkylation: see for example: a) U. Lücking et al, US 2007/0232632; b) C. R. Johnson, J. Org. Chem. 1993, 58, 1922; c) C. Bolm et al, Synthesis 2009, 10, 1601.
Acylation: see for example: a) C. Bolm et al, Chem. Europ. J. 2004, 10, 2942; b) C. Bolm et al, Synthesis 2002, 7, 879; c) C. Bolm et al, Chem. Europ. J. 2001, 7, 1118.
Arylation: see for example: a) C. Bolm et al, Tet. Lett. 1998, 39, 5731; b) C. Bolm et al., J. Org. Chem. 2000, 65, 169; c) C. Bolm et al, Synthesis 2000, 7, 911; d) C. Bolm et al, J. Org. Chem. 2005, 70, 2346; e) U. Lücking et al, WO2007/71455.
Reaction with isocyanates: see for example: a) V. J. Bauer et al, J. Org. Chem. 1966, 31, 3440; b) C. R. Johnson et al, J. Am. Chem. Soc. 1970, 92, 6594; c) S. Allenmark et al, Acta Chem. Scand. Ser. B 1983, 325; d) U. Lücking et al, US2007/0191393.
Reaction with sulfonylchlorides: see for example: a) D. J. Cram et al, J. Am. Chem. Soc. 1970, 92, 7369; b) C. R. Johnson et al, J. Org. Chem. 1978, 43, 4136; c) A. C. Barnes, J. Med. Chem. 1979, 22, 418; d) D. Craig et al, Tet. 1995, 51, 6071; e) U. Lücking et al, US2007/191393.
Reaction with chloroformiates: see for example: a) P. B. Kirby et al, DE2129678; b) D. J. Cram et al, J. Am. Chem. Soc. 1974, 96, 2183; c) P. Stoss et al, Chem. Ber. 1978, 111, 1453; d) U. Lücking et al, WO2005/37800.

An alternative route for the preparation of compounds of general formula (Ia) is described in Scheme 31.

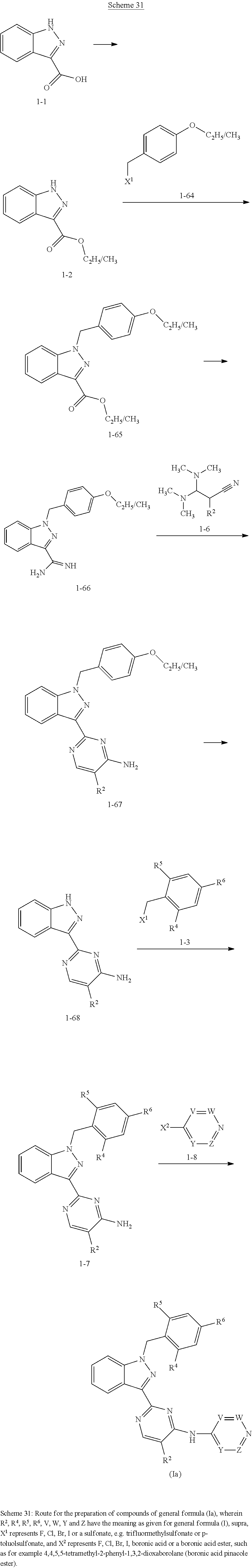

In addition, interconversion of any of the substituents R², R⁴, R⁵, R⁶, V, W, Y or Z can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999). Specific examples are described in the subsequent paragraphs.

Compounds 1-3, 1-6 and 1-8 are either commercially available or can be prepared according to procedures available from the public domain, as understandable to the person skilled in the art. Specific examples are described in the subsequent paragraphs.

A suitably substituted 1H-indazole-3-carboxylic acid of the general formula (1-1) can be reacted with methanol or ethanol in the presence of catalytic amounts of a Broensted acid, such as, for example, hydrochloric acid or sulphuric acid, at temperatures ranging from 0° C. to boiling point of the respective alcohol, preferably the reaction is carried out at 85° C., to furnish alkyl 1H-indazole-3-carboxylate intermediates of general formula (1-2).

Alkyl 1H-indazole-3-carboxylate Intermediates of the general formula (1-2) can be converted to intermediates of general formula (1-65) by reaction with a suitable alkylating agent, such as, for example a substituted benzyl halide (1-64), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-65) are treated with the reagent methylchloroaluminiumamide prepared in situ by addition of ammonium chloride to commercially available trimethylaluminium, in a suitable solvent system, such as, for example, toluene, at a temperature between 0° C. and the boiling point of the respective solvent, preferably the reaction is carried out at 80° C. and are quenched with a suitable solvent system, such as, for example, methanol, to form the desired intermediate of general formula (1-66).

Intermediates of general formula (1-66) can be converted to intermediates of general formula (1-67) by reaction with a suitably substituted 3,3-bis(dimethylamino)propanenitrile of the general formula (1-6), such as, for example 3,3-bis(dimethylamino)-2-methoxypropanenitrile, in the presence of a suitable base, such as, for example piperidine, in a suitable solvent system, such as, for example, 3-methylbutan-1-ol, in a temperature range from room temperature to the boiling point of the respective solvent, preferably the reaction is carried out at 100° C.

Intermediates of general formula (1-67) can be converted to intermediates of general formula (1-68) by reaction with a suitably Broensted acid, such as, for example methanesulfonic acid and trifluoroacetic acid, in a suitable solvent system, such as, for example, dichloromethane, in a temperature range from room temperature to the boiling point of the respective solvent, preferably the reaction is carried out at room temperature.

Intermediates of the general formula (1-68) can be converted to intermediates of general formula (1-7) by reaction with a suitable alkylating agent, such as, for example a substituted benzyl halide (1-3), in the presence of a suitable base, such as, for example sodium hydride, in a suitable solvent system, such as, for example, DMF, at a temperature between −20° C. and boiling point of the respective solvent, preferably the reaction is carried out at 0° C.

Intermediates of general formula (1-7) can be reacted with a suitable six membered heterocycle of the general formula (1-8), such as, for example 4-bromo-2-methyl-pyridine, in the presence of a suitable base, such as, for example sodium 2-methylpropan-2-olate, and a suitable palladium catalyst, such as for example (1E,4E)-1,5-diphenylpenta-1,4-dien-3-one-palladium, in the presence of a suitable ligand, such as for example 1′-binaphthalene-2,2′-diylbis(diphenylphosphane), in a suitable solvent system, such as, for example, DMF, in a temperature range from room temperature to the boiling point of the respective solvent, preferably the reaction is carried out at at 100° C. to furnish compounds of general formula (Ia). Alternatively the following palladium catalysts can be used: allylpalladium chloride dimmer, dichlorobis(benzonitrile)palladium (II), palladium (II) acetate, palladium (II) chloride, tetrakis(triphenylphosphine)palladium (0), tris(dibenzylideneacetone)dipalladium (0), chloro(2′-amino-1,1′-biphenyl-2-yl)palladium(II) dimer, (2′-amino-1,1′-biphenyl-2-yl)methanesulfonatopalladium(II) dimer, trans-di(p-acetato)bis[o-(di-o-tolylphosphino)benzyl]dipalladium(II) [cataCXium® C], allylchloro[1,3-bis(2,4,6-trimethylphenyl)imidazol-2-ylidene]palladium(II), allylchloro[1,3-bis(2,6-diisopropylphenyl)imidazol-2-ylidene]palladium(II), chloro[(1,3-dimesitylimidazol-[1,3-bis(2,4,6-trimethylphenyl)-1,3-dihydro-2H-imidazol-2-ylidene](chloro){2-[(dimethylamino)methyl]phenyl}palladium, chloro[(1,2,3-N)-3-phenyl-2-propenyl][1,3-bis(2,6-di-iso-propylphenyl)imidazol-2-ylidene]palladium(II), [2-(acetylamino)phenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]phenyl} palladium, {1,3-bis[2,6-di(propan-2-yl)phenyl]-2,3-dihydro-1H-imidazol-2-yl}(dichloro)(3-chloropyridine-kappaN)palladium, [1,3-bis(2,6-diisopropylphenyl) imidazol-2-ylidene](3-chloropyridyl)palladium(II) dichloride, [2-(acetylamino)-4-methoxyphenyl]{1,3-bis[2,6-di(propan-2-yl)phenyl]-1,3-dihydro-2H-imidazol-2-ylidene}chloropalladium, {1,3-bis[2,6-di(propan-2-yl)phenyl]-1,3-dihydro-2H-imidazol-2-ylidene}(chloro){2-[(dimethylamino)methyl]-3,5-dimethoxyphenyl}palladium, dichloro[1,3-bis(2,6-di-3-pentylphenyl)imidazol-2-ylidene](3-chloropyridyl) palladium(II), dichloro(di-μ-chloro)bis[1,3-bis(2,6-di-iso-propylphenyl) imidazol-2-ylidene]dipalladium(II), 2-(2′-di-tert-butylphosphine)biphenylpalladium(II) acetate, chloro[dicyclohexyl(2′,6′-dimethoxybiphenyl-2-yl)-lambda5-phosphanyl][2-(phenyl-kappaC2)ethanaminato-kappaN]palladium, [2-(2-aminoethyl)phenyl](chloro)palladium-di-tert-butyl[2′,4′,6′-tri(propan-2-yl)biphenyl-2-yl]phosphane, {dicyclohexyl[2′,4′,6′-tri(propan-2-yl)biphenyl-2-yl]phosphane}{2-[2-(methylazanidyl-kappaN)ethyl]phenyl-kappaC1}palladium, chloro(2-dicyclohexylphosphino-2′,6′-dimethoxy-1,1′-biphenyl)(2′-amino-1,1′-biphenyl-2-yl) palladium(II), [2′,6′-bis(propan-2-yloxy)biphenyl-2-yl](dicyclohexyl)phosphane-[2-(2-aminoethyl)phenyl](chloro)palladium, [2-(2-aminoethyl)phenyl](chloro){dicyclohexyl[2′,4′,6′-tri(propan-2-yl)biphenyl-2-yl]-lambda5-phosphanylidene}palladium, 2′-(dicyclohexylphosphanyl)-N,N,N′,N′-tetramethylbiphenyl-2,6-diamine-(2′-aminobiphenyl-2-yl)(chloro)palladium, chloro(2-dicyclohexylphosphino-2′,6′-di-iso-propoxy-1,1′-biphenyl)(2-amino-1,1′-biphenyl-2-yl)palladium(II), [2′-(azanidyl-kappaN)biphenyl-2-yl-kappaC2](chloro){dicyclohexyl [2′,4′,6′-tri(propan-2-yl)biphenyl-2-yl]-lambda5-phosphanyl}palladium, (2′-aminobi-phenyl-2-yl)(methanesulfonato-kappaO)palladium-di-tert-butyl[2′,4′,6′-tri(propan-2-yl)biphenyl-2-yl]phosphane, (2′-aminobiphenyl-2-yl)palladium(1+) methanesulfonate-di-tert-butyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-[2-(2-aminoethyl) phenyl](chloro)palladium, (2′-aminobiphenyl-2-yl)palladium(1+) methanesulfonate-2′-(dicyclohexylphosphanyl)-N,N,N′,N′-tetramethylbiphenyl-2, 6-diamine, sodium 2′-(dicyclohexylphosphanyl)-2,6-dimethoxybiphenyl-3-sulfonate-(2′-aminobiphenyl-2-yl)(chloro)palladium, chloro(2-dicyclohexylphosphino-2′,4′,6′-tri-iso-propyl-1,1′-biphenyl)[2-(2-aminoethyl)phenyl]palladium(II), (2′-aminobiphenyl-2-yl)(methane-sulfonato-kappaO)palladium-[2′,6′-bis(propan-2-yloxy)biphenyl-2-yl](dicyclohexyl) phosphane, (2′-aminobiphenyl-2-yl)(methanesulfonato-kappaO)palladium-dicyclohexyl[2′,4′,6′-tri(propan-2-yl)biphenyl-2-yl]phosphane, (2′-aminobiphenyl-2-yl)palladium(1+) methanesulfonate-dicyclohexyl[2′,4′,6′-tri(propan-2-yl)biphenyl-2-yl]phosphane, dicyclohexyl[3,6-dimethoxy-2′,4′,6′-tri(propan-2-yl)biphenyl-2-yl]phosphane-(2′-aminobiphenyl-2-yl) (chloro)palladium, (2′-aminobiphenyl-2-yl)(methanesulfonato-kappaO)palladium-di-tert-butyl[3,6-dimethoxy-2′,4′,6′-tri(propan-2-yl)biphenyl-2-yl]phosphane, (2′-aminobiphenyl-2-yl)(methanesulfonato-kappaO)palladium dicyclohexyl[3,6-dimethoxy-2′,4′,6′-tri(propan-2-yl)biphenyl-2-yl]phosphane or the following ligands:

racemic-2,2′-bis(diphenylphosphino)-1,1′-binaphthyl, rac-BINAP, 1,1′-bis(diphenyl-phosphino)ferrocene, bis(2-diphenylphosphinophenyl)ether, di-tert-butylmethylphosphonium tetrafluoroborate, 2-(di-tert-butylphosphino)biphenyl, tri-tert-butylphosphonium tetrafluoroborate, tri-2-furylphosphine, tris(2,4-di-tert-butylphenyl)phosphite, tri-o-tolylphosphine, (9,9-dimethyl-9H-xanthene-4,5-diyl)bis(diphenylphosphine), dicyclohexyl(2′,4′,6′-triisopropyl-3,6-dimethoxybiphenyl-2-yl)phosphine, di-tert-butyl (2′,4′,6′-triisopropyl-3,6-dimethoxybiphenyl-2-yl)phosphine, di-tert-butyl(2′,4′,6′-triiso propylbiphenyl-2-yl)phosphine, dicyclohexyl(2′,4′,6′-triisopropylbiphenyl-2-yl) phosphine, di-tert-butyl(2′,4′,6′-triisopropyl-3-methoxy-6-methylbiphenyl-2-yl)phos-phine, di-tert-butyl(2′,4′,6′-triisopropyl-3,4,5,6-tetramethylbiphenyl-2-yl) phosphine, adamantan-1-yl(adamantan-2-yl)(2′,4′,6′-triisopropyl-3,6-dimethoxybiphenyl-2-yl) phosphine, dicyclohexyl(2′,6′-dimethoxybiphenyl-2-yl)phosphine, dicyclohexyl(2′,6′-diisopropoxybiphenyl-2-yl)phosphine, 2′-(dicyclohexylphosphino)-N,N-dimethyl-biphenyl-2-amine, 2′-(di-tert-butylphosphino)-N,N-dimethylbiphenyl-2-amine, 2′-(di-phenylphosphino)-N,N,N′,N′-tetramethylbiphenyl-2,6-diamine, di-tert-butyl(2′,4′,6′-tricyclohexyl-3,6-dimethoxybiphenyl-2-yl)phosphine, bis[3,5-bis(trifluoromethyl)phe-nyl] (2′,4′,6′-triisopropyl-3,6-dimethoxybiphenyl-2-yl)phosphine, biphenyl-2-yl(di-tert-butyl)phosphine, dicyclohexyl(2′-methylbiphenyl-2-yl)phosphine, biphenyl-2-yl (dicyclohexyl)phosphine, 2′-(dicyclohexylphosphino)-N,N-dimethylbiphenyl-2-amine, 2′-(dicyclohexylphosphino)-N,N,N′,N′-tetramethylbiphenyl-2,6-diamine, sodium 2′-(dicyclohexylphosphino)-2,6-diisopropylbiphenyl-4-sulfonate, sodium 2′-(dicyclohexylphosphino)-2,6-dimethoxybiphenyl-3-sulfonate, 1,1′-binaphthalen-2-yl(di-tert-butyl)phosphine, 1,3-bis(2,4,6-trimethylphenyl)-1,3-dihydro-2H-imidazol-2-ylidene, 1,3-bis[2,6-di(propan-2-yl)phenyl]-1,3-dihydro-2H-imidazol-2-ylidene.

Alternatively intermediates of general formula (1-7) can be reacted with a suitable boronic acid or boronic acid pinacole ester of general formula (1-8), such as, for example (2-fluoropyridin-4-yl)boronic acid, in the presence of a suitable base, such as, for example triethylamine, a suitable activating agent such as for example N,N-dimethylpyridin-4-amine and a suitable copper salt, such as for example copper (II) acetate, in a suitable solvent system, such as, for example, trichloromethane, in a temperature range from room temperature to the boiling point of the respective solvent, preferably the reaction is carried out at room temperature to furnish compounds of general formula (Ia).

Alternatively intermediates of general formula (1-7) can be reacted with a suitable six membered heterocycle of the general formula (1-8), such as for example 4-fluoro-2-methyl-pyridine, in the presence of a suitable base, such as, for example sodium hydride, in a suitable solvent system, such as, for example DMF, in a temperature range from room temperature to the boiling point of the respective solvent, preferably the reaction is carried out at 90° C. to furnish compounds of general formula (Ia).

Compounds of general formula (1-69) can be converted into compounds of general formula (1-71) according to the procedure depicted in Scheme 32.

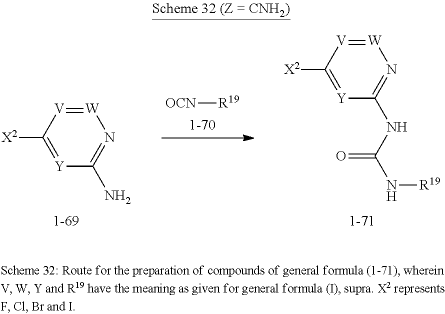

Intermediates of general formula (1-69) can be converted to intermediates of general formula (1-71) by reaction with isocyanate derivative (1-70), in a suitable solvent system, such as, for example, THF, in a temperature range from room temperature to the boiling point of the respective solvent, preferably the reaction is carried out at 70° C.

It is known to the person skilled in the art that, if there are a number of reactive centers on a starting or intermediate compound, it may be necessary to block one or more reactive centers temporarily by protective groups in order to allow a reaction to proceed specifically at the desired reaction center. A detailed description for the use of a large number of proven protective groups is found, for example, in T. W. Greene, Protective Groups in Organic Synthesis, John Wiley & Sons, 1999, 3rd Ed., or in P. Kocienski, Protecting Groups, Thieme Medical Publishers, 2000.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present invention which possess a sufficiently basic or acidic functionality, may result in the formation of a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, solvate, inclusion complex) of a compound of the present invention as isolated as described herein is not necessarily the only form in which said compound can be applied to a biological assay in order to quantify the specific biological activity.

Salts of the compounds of formula (I) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the examples section.

Pure diastereomers and pure enantiomers of the compounds and salts according to the invention can be obtained e.g. by asymmetric synthesis, by using chiral starting compounds in synthesis and by splitting up enantiomeric and diasteriomeric mixtures obtained in synthesis.

Enantiomeric and diastereomeric mixtures can be split up into the pure enantiomers and pure diastereomers by methods known to a person skilled in the art. Preferably, diastereomeric mixtures are separated by crystallization, in particular fractional crystallization, or chromatography. Enantiomeric mixtures can be separated e.g. by forming diastereomers with a chiral auxiliary agent, resolving the diastereomers obtained and removing the chiral auxiliary agent. As chiral auxiliary agents, for example, chiral acids can be used to separate enantiomeric bases such as e.g. mandelic acid and chiral bases can be used to separate enantiomeric acids via formation of diastereomeric salts. Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxiliary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

One preferred aspect of the invention is the process for the preparation of the compounds of claims 1-6 according to the examples.

Optionally, compounds of the formula (I) can be converted into their salts, or, optionally, salts of the compounds of the formula (I) can be converted into the free compounds. Corresponding processes are customary for the skilled person.

Optionally, compounds of the formula (I) can be converted into their N-oxides. The N-oxide may also be introduced by way of an intermediate. N-oxides may be prepared by treating an appropriate precursor with an oxidizing agent, such as meta-chloroperbenzoic acid, in an appropriate solvent, such as DCM, at suitable temperatures, such as from 0° C. to 40° C., whereby room temperature is generally preferred. Further corresponding processes for forming N-oxides are customary for the skilled person.

One preferred aspect of the invention is the process for the preparation of the compounds of claims 1-6 according to the examples, as well as the intermediates used for their preparation.

Optionally, compounds of the formula (I) can be converted into their salts, or, optionally, salts of the compounds of the formula (I) can be converted into the free compounds. Corresponding processes are customary for the skilled person.

Commercial Utility

As mentioned supra, the compounds of the present invention have surprisingly been found to effectively inhibit Bub1 finally resulting in cell death e.g. apoptosis and may therefore be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival, inappropriate cellular immune responses, or inappropriate cellular inflammatory responses is mediated by Bub1, such as, for example, benign and malignant neoplasia, more specifically haematological tumours, solid tumours, and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof,

especially haematological tumours, solid tumours, and/or metastases of breast, bladder, bone, brain, central and peripheral nervous system, cervix, colon, endocrine glands (e.g. thyroid and adrenal cortex), endocrine tumours, endometrium, esophagus, gastrointestinal tumours, germ cells, kidney, liver, lung, larynx and hypopharynx, mesothelioma, ovary, pancreas, prostate, rectum, renal, small intestine, soft tissue, stomach, skin, testis, ureter, vagina and vulva as well as malignant neoplasias including primary tumors in said organs and corresponding secondary tumors in distant organs (“tumor metastases”). Haematological tumors can e.g be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

A further aspect of the invention is the use of the compounds according to formula (I) for the treatment of cer-vical-, breast-, non-small cell lung-, prostate-, colon- and melanoma tumors and/or metastases thereof, especially preferred for the treatment thereof as well as a method of treatment of cervical-, breast-, non-small cell lung-, prostate-, colon- and melanoma tumors and/or metastases thereof comprising administering an effective amount of a compound of formula (I).

One aspect of the invention is the use of the compounds according to formula (I) for the treatment of cervix tumors as well as a method of treatment of cervix tumors comprising administering an effective amount of a compound of formula (I).

In accordance with an aspect of the present invention therefore the invention relates to a compound of general formula I, or an N-oxide, a salt, a tautomer or a stereoisomer of said compound, or a salt of said N-oxide, tautomer or stereoisomer particularly a pharmaceutically acceptable salt thereof, or a mixture of same, as described and defined herein, for use in the treatment or prophylaxis of a disease, especially for use in the treatment of a disease.

Another particular aspect of the present invention is therefore the use of a compound of general formula I, described supra, or a stereoisomer, a tautomer, an N-oxide, a hydrate, a solvate, or a salt thereof, particularly a pharmaceutically acceptable salt thereof, or a mixture of same, for the prophylaxis or treatment of hyperproliferative disorders or disorders responsive to induction of cell death i.e apoptosis.

The term “inappropriate” within the context of the present invention, in particular in the context of “inappropriate cellular immune responses, or inappropriate cellular inflammatory responses”, as used herein, is to be understood as preferably meaning a response which is less than, or greater than normal, and which is associated with, responsible for, or results in, the pathology of said diseases.

Preferably, the use is in the treatment or prophylaxis of diseases, especially the treatment, wherein the diseases are haematological tumours, solid tumours and/or metastases thereof.

Another aspect is the use of a compound of formula (I) is for the treatment of cervical-, breast-, non-small cell lung-, prostate-, colon- and melanoma tumors and/or metastases thereof, especially preferred for the treatment thereof. A preferred aspect is the use of a compound of formula (I) for the prophylaxis and/or treatment of cervical tumors especially preferred for the treatment thereof.

Another aspect of the present invention is the use of a compound of formula (I) or a stereoisomer, a tautomer, an N-oxide, a hydrate, a solvate, or a salt thereof, particularly a pharmaceutically acceptable salt thereof, or a mixture of same, as described herein, in the manufacture of a medicament for the treatment or prophylaxis of a disease, wherein such disease is a hyperproliferative disorder or a disorder responsive to induction of cell death e.g. apoptosis. In an embodiment the disease is a haematological tumour, a solid tumour and/or metastases thereof. In another embodiment the disease is cervical-, breast-, non-small cell lung-, prostate-, colon- and melanoma tumor and/or metastases thereof, in a preferred aspect the disease is cervical tumor.

Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce cell death e.g. apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death e.g. apoptosis of such cell types.

Preferably, the diseases of said method are haematological tumours, solid tumour and/or metastases thereof.

The compounds of the present invention can be used in particular in therapy and prevention i.e. prophylaxis, especially in therapy of tumour growth and metastases, especially in solid tumours of all indications and stages with or without pre-treatment of the tumour growth.

Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease.

Therefore, the present invention includes pharmaceutical compositions that are comprised of a pharmaceutically acceptable carrier or auxiliary and a pharmaceutically effective amount of a compound, or salt thereof, of the present invention.

Another aspect of the invention is a pharmaceutical composition comprising a pharmaceutically effective amount of a compound of formula (I) and a pharmaceutically acceptable auxiliary for the treatment of a disease mentioned supra, especially for the treatment of haematological tumours, solid tumours and/or metastases thereof.

A pharmaceutically acceptable carrier or auxiliary is preferably a carrier that is non-toxic and innocuous to a patient at concentrations consistent with effective activity of the active ingredient so that any side effects ascribable to the carrier do not vitiate the beneficial effects of the active ingredient. Carriers and auxiliaries are all kinds of additives assisting to the composition to be suitable for administration.

A pharmaceutically effective amount of compound is preferably that amount which produces a result or exerts the intended influence on the particular condition being treated.

The compounds of the present invention can be administered with pharmaceutically-acceptable carriers or auxiliaries well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing auxiliaries, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for administration, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);
aerosol propellants (examples include but are not limited to carbon dioxide, CCl₂F₂, F₂ClC—CClF₂ and CClF₃)

air displacement agents—examples include but are not limited to nitrogen and argon;

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate),

flavourants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas),

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, crosslinked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile i.v. solution: A 5 mg/mL solution of the desired compound of this invention can be made using sterile, injectable water, and the pH is adjusted if necessary. The solution is diluted for administration to 1-2 mg/mL with sterile 5% dextrose and is administered as an i.v. infusion over about 60 minutes.

Lyophilised powder for i.v. administration: A sterile preparation can be prepared with (i) 100-1000 mg of the desired compound of this invention as a lyophilised powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.

Intramuscular suspension: The following solution or suspension can be prepared, for intramuscular injection:

50 mg/mL of the desired, water-insoluble compound of this invention

5 mg/mL sodium carboxymethylcellulose

4 mg/mL TWEEN 80

9 mg/mL sodium chloride

9 mg/mL benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Combination Therapies

The compounds of this invention can be administered as the sole pharmaceutical agent or in combination with one or more other pharmaceutical agents where the combination causes no unacceptable adverse effects. Those combined pharmaceutical agents can be other agents having antiproliferative effects such as for example for the treatment of haematological tumours, solid tumours and/or metastases thereof and/or agents for the treatment of undesired side effects. The present invention relates also to such combinations.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, especially (chemotherapeutic) anti-cancer agents as defined supra. The combination can be a non-fixed combination or a fixed-dose combination as the case may be.

Methods of testing for a particular pharmacological or pharmaceutical property are well known to persons skilled in the art.

The example testing experiments described herein serve to illustrate the present invention and the invention is not limited to the examples given.

As will be appreciated by persons skilled in the art, the invention is not limited to the particular embodiments described herein, but covers all modifications of said embodiments that are within the spirit and scope of the invention as defined by the appended claims.

The following examples illustrate the invention in greater detail, without restricting it. Further compounds according to the invention, of which the preparation is not explicitly described, can be prepared in an analogous way.

The compounds, which are mentioned in the examples and the salts thereof represent preferred embodiments of the invention as well as a claim covering all subcombinations of the residues of the compound of formula (I) as disclosed by the specific examples.

The term “according to” within the experimental section is used in the sense that the procedure referred to is to be used “analogously to”.

### Experimental Part

The following table lists the abbreviations used in this paragraph and in the Intermediate Examples and Examples section as far as they are not explained within the text body.

| Abbreviation | Meaning |
|---|---|
| ACN | acetonitrile |
| aq. | aqueous |
| alloc | allyloxycarbonyl |
| boc | tert-butoxycarbonyl |
| br | broad |
| CI | chemical ionisation |
| d | doublet |
| dd | doublet of doublet |
| DAD | diode array detector |
| DCM | dichloromethane |
| DIPEA | N-ethyl-N-(propan-2-yl)propan-2-amine (CAS number |
| | 7087-68-5) |
| DMF | N,N-dimethylformamide |
| EtOAc | ethyl acetate |
| eq. | equivalent |
| ESI | electrospray (ES) ionisation |
| h | hour |
| HATU | 2-(7-aza-1H-benzotriazole-1-yl)-1,1,3,3-tetramethyl- |
| | uronium hexafluorophosphate (CAS number 148893-10- |
| | 1) |
| HPLC | high performance liquid chromatography |
| LC-MS | liquid chromatography mass spectrometry |
| m | multiplet |
| min | minute |
| MS | mass spectrometry |
| n-BuLi | n-butyllithium |
| NMR | nuclear magnetic resonance spectroscopy: chemical |
| | shifts (δ) are given in ppm. The chemical shifts were |
| | corrected by setting the DMSO signal to 2.50 ppm using |
| | unless otherwise stated. |
| PoraPak ™; | a HPLC column obtainable from Waters |
| PYBOP | (1H-benzotriazol-1-yloxy)(tripyrrolidin-1-yl)phosphonium |
| | hexafluorophosphate (CAS number 128625-52-5) |
| q | quartet |
| r.t. or rt | room temperature |
| RT | retention time (as measured either with HPLC or UPLC) |
| | in minutes |
| s | singlet |
| SM | starting material |
| t | triplet |
| THF | tetrahydrofuran |
| UPLC | ultra performance liquid chromatography |

Other abbreviations have their meanings customary per se to the skilled person. The various aspects of the invention described in this application are illustrated by the following examples which are not meant to limit the invention in any way.

Specific Experimental Descriptions

NMR peak forms in the following specific experimental descriptions are stated as they appear in the spectra, possible higher order effects have not been considered. Reactions employing microwave irradiation may be run with a Biotage Initator® microwave oven optionally equipped with a robotic unit. The reported reaction times employing microwave heating are intended to be understood as fixed reaction times after reaching the indicated reaction temperature. The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel or Isolute® Flash NH₂ silica gel in combination with a Isolera autopurifier (Biotage) and eluents such as gradients of e.g. hexane/EE or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and ACN which may contain additives such as trifluoroacetic acid, formic acid or aqueous ammonia. In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The percentage yields reported in the following examples are based on the starting component that was used in the lowest molar amount. Air and moisture sensitive liquids and solutions were transferred via syringe or cannula, and introduced into reaction vessels through rubber septa. Commercial grade reagents and solvents were used without further purification. The term “concentrated in vacuo” refers to use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

Analytical LC-MS Conditions

LC-MS-data given in the subsequent specific experimental descriptions refer (unless otherwise noted) to the following conditions:

| | |
|---|---|
| System: | Waters Acquity UPLC-MS: Binary Solvent Manager, |
| | Sample Manager/Organizer, Column Manager, PDA, |
| | ELSD, SQD 3001 or ZQ4000 |
| System: | Waters Acquity UPLC-MS: Binary Solvent Manager, |
| | Sample Manager/Organizer, PDA, ELSD, |
| Column: | Acquity UPLC BEH C18 1.7 50 × 2.1 mm |
| Solvent: | A1 = water + 0.1% formic acid |
| | A2 = water + 0.2% ammonia |
| | B1 = acetonitrile |
| Gradient: | 0-1.6 min 1-99% B, 1.6-2.0 min 99% B |
| Flow: | 0.8 mL/min |
| Temperatuer: | 60° C. |
| Injektion: | 2.0 μL |
| Detection: | DAD scan range 210-400 nm -> Peaktable |
| | ELSD |
| | MS ESI+, ESI− Switch -> variouse scan ranges |
| Methods: | Method 1: Mass_100_1000 |
| | Method 2: Mass_160_1000 |
| | Method 3: Mass_160_2000 |
| | Method 4: Mass_160_1000_BasicReport |
| | Method 5: NH₃_Mass_100_1000 |
| | Method 6: NH₃_Mass_160_1000 |

Preparative HPLC Conditions

“Purification by preparative HPLC” in the subsequent specific experimental descriptions refers to (unless otherwise noted) the following conditions:

Analytics(Pre- and Post Analytics: Method B:

| | |
|---|---|
| System: | Waters Acquity UPLC-MS: Binary Solvent Manager, |
| | Sample Manager/Organizer, Column Manager, PDA, |
| | ELSD, SQD 3001 |
| Column: | Aqcuity BEH C18 1.7 50 × 2.1 mm |
| Solvent: | A = water + 0.1% formic acid |
| | B = acetonitrile |
| Gradient: | 0-1.6 min 1-99% B, 1.6-2.0 min 99% B |
| Flow: | 0.8 mL/min |
| Temperature: | 60° C. |
| Injection: | 2.0 μL |
| Detection: | DAD scan range 210-400 nm |
| | MS ESI+, ESI−, scan range 160-1000 m/z |
| | ELSD |

Preparation:

| | |
|---|---|
| System: | Waters Autopurificationsystem: Pump 2545, Sample |
| | Manager 2767, CFO, |
| | DAD 2996, ELSD 2424, SQD 3001 |
| Column: | XBrigde C18 5 μm 100 × 30 mm |
| Solvent: | A = water + 0.1% formic acid |
| | B = acetonitrile |
| Gradient: | 0-1 min 1% B, 1-8 min 1-99% B, 8-10 min 99% B |
| Flow: | 50 mL/min |
| Temperature: | RT |
| Solution: | max. 250 mg/2.5 mL dimethyl sufoxide or DMF |
| Injection: | 1 × 2.5 mL |
| Detection: | DAD scan range 210-400 nm |
| | MS ESI+, ESI−, scan range 160-1000 m/z |

Chiral HPLC Conditions

Chiral HPLC-data given in the subsequent specific experimental descriptions refer to the following conditions:

Analytics:

| | |
|---|---|
| System: | Dionex: Pump 680, ASI 100, Waters: UV-Detektor 2487 |
| Column: | Chiralpak IC 5 μm 150 × 4.6 mm |
| Solvent: | hexane/ethanol 80:20 + 0.1% diethylamine |
| Flow: | 1.0 mL/min |
| Temperature: | 25° C. |
| Solution: | 1.0 mg/mL ethanol/methanol 1:1 |
| Injection: | 5.0 μL |
| Detection: | UV 280 nm |

Preparation:

| | |
|---|---|
| System: | Agilent: Prep 1200, 2xPrep Pump, DLA, MWD, Prep FC, |
| | ESA: Corona |
| Column: | Chiralpak IC 5 μm 250 × 30 mm |
| Solvent: | hexane/ethanol 80:20 + 0.1% diethylamine |
| Flow: | 40 mL/min |
| Temperature: | RT |
| Solution: | 660 mg/5.6 mL ethanol |
| Injection: | 8 × 0.7 mL |
| Detection: | UV 280 nm |

Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

Determination of Optical Rotation Conditions

Optical rotations were measured in dimethyl sulfoxide at 589 nm wavelength, 20° C., concentration 1.0000 g/100 mL, integration time 10 s, film thickness 100.00 mm.

### EXAMPLES

### Synthetic Intermediates

### Intermediate 1-1-1

### Preparation of 1-(4-ethoxy-2,6-difluorobenzyl)-1H-indazole-3-carboximidamide hydrochloride 1:1

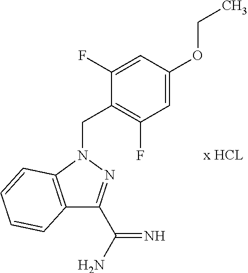 [SMILES: CCOc1cc(F)c(Cn2nc(C(=N)N)c3ccccc32)c(F)c1]

58 g of ammonium chloride (1083 mmol, 5.0 eq.) were suspended in 1 L of dry toluene under nitrogen atmosphere and cooled down to 0° C. bath temperature. 541 mL of 2M trimethylaluminium solution in toluene (1083 mmol, 5.0 eq.) were added drop wise. The mixture was stirred at room temperature until disappearance of gassing. 75 g of methyl 1-(4-ethoxy-2,6-difluorobenzyl)-1H-indazole-3-carboxylate 1-2-1 (59.8 mmol, 1.0 eq.) were dissolved in 1 L of dry toluene and added drop wise to the reaction mixture and stirred over night at 80° C. bath temperature. The mixture was cooled down with an ice bath to 0° C. bath temperature, 1.4 L of methanol were added and stirred for one hour at rt. The resulting suspension was filtered over celite and washed with methanol. The filtrate was concentrated in vacuo and dried in vacuo at 50° C. and the crude product was used without any further purification: 67.3 g (183 mmol, 84%).

¹H NMR (300 MHz, DMSO-d₆) δ[ppm]=1.26 (t, 3H), 4.01 (q, 2H), 5.75 (s, 2H), 6.68-6.78 (m, 2H), 7.34-7.43 (m, 1H), 7.56-7.61 (m, 1H), 7.93 (dd, 2H), 9.29 (br. s, 4H).

The following intermediate was prepared according to the same procedure from the indicated starting materials (SM=starting material):

| | | | |
|---|---|---|---|
| 1-1-2 SM = 1-19-1 | 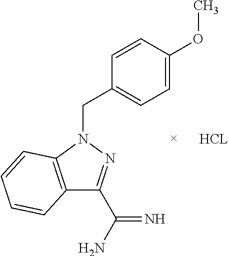 | 1-(4- methoxybenzyl)- 1H-indazole-3- carboximidamide hydrochloride | ¹H NMR (300 MHz, DMSO-d₆) δ [ppm] = 3.62-3.70 (s, 3 H), 5.57 (s, 2 H), 6.37 (br. s., 3 H), 6.78-6.88 (m, 2 H), 7.10- 7.23 (m, 3 H), 7.35 (ddd, 1 H), 7.68 (d, 1 H), 8.27 (d, 1 H). |
| 1-1-3 SM = 1-33-1 | 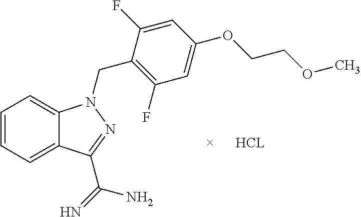 | 1-[2,6-difluoro-4- (2- methoxyethoxy) benzyl]-1H- indazole-3- carboximidamide hydrochloride | ¹H NMR (400 MHz, DMSO-d₆) δ [ppm] = 3.27 (s, 3H), 3.58- 3.64 (m, 2H), 4.08-4.14 (m, 2H), 5.78 (s, 2H), 6.76-6.84 (m, 2H), 7.37-7.45 (m, 1H), 7.58-7.64 (m, 1H), 7.78 (br. s, 4H), 7.93 (d, 1H), 7.98 (d, 1H). |

### Intermediate 1-2-1

### Preparation of methyl 1-(4-ethoxy-2,6-difluorobenzyl)-1H-indazole-3-carboxylate

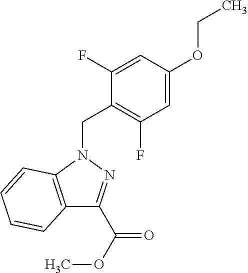 [SMILES: CCOc1cc(F)c(Cn2nc(C(=O)OC)c3ccccc32)c(F)c1]

185 g of methyl 1H-indazole-3-carboxylate (1050 mmol, 1.0 eq.) were dissolved in 3 l of dry THF and cooled to 5° C. 411 g of cesium carbonate (1260 mmol, 1.2 eq.) were added stirred for 15 min. 290 g of 2-(bromomethyl)-5-ethoxy-1,3-difluorobenzene (1155 mmol, 1.1 eq.) dissolved in 250 ml THF were added drop wise at 5° C. The precipitate was filtered off. The filtrate was concentrated in vacuo. The residue was crystallized from EE/hexane (1:1) to provide 310 g (895 mmol, 85%) of analytically pure target compound. 1H NMR (400 MHz, DMSO-d₆) δ [ppm]=1.27 (t, 3H), 3.86 (s, 3H), 4.01 (q, 2H), 5.68 (s, 2H), 6.70-6.76 (m, 2H), 7.32 (t, 1H), 7.50 (t, 1H), 7.84 (d, 1H), 8.00-8.12 (m, 1H).

### Intermediate 1-3-1

### Preparation of 2-[1-(4-ethoxy-2,6-difluorobenzyl)-1H-indazol-3-yl]-5-[2-(methylsulfanyl)ethyl]pyrimidin-4-amine

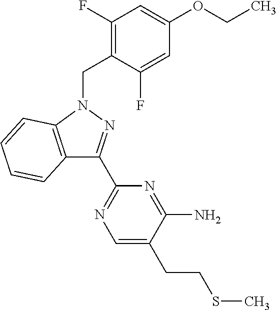 [SMILES: CCOc1cc(F)c(Cn2nc(-c3ncc(CCSC)c(N)n3)c3ccccc32)c(F)c1]

7.42 g of 1-(4-ethoxy-2,6-difluorobenzyl)-1H-indazole-3-carboximidamide hydrochloride 1:1 1-1-1 (20.2 mmol, 1.0 eq.), 6.89 g of (2Z)-2-[(dimethylamino)methylene]-5-(methylsulfanyl)pentanenitrile 1-4-1 (40.5 mmol, 2.0 eq.) and 3.02 mL of 1,8-Diazabicyclo(5.4.0)undec-7-en (20.2 mmol, 1.0 eq.) were dissolved in 180 mL pyridine. The mixture was stirred over night at 110° C. The re action mixture was poured into icewater and extracted three times with DCM. The combined organic layers were dried using a waterresistant filter and the filtrate was dried under reduced pressure. The crude product was purified by flash chromatography to provide the desired compound in 96% purity: 940 mg, 2.0 mmol, 9.8%.

¹H-NMR (300 MHz, DMSO-d₆): δ [ppm]=1.28 (t, 3H), 2.08 (s, 3H), 2.63-2.79 (m, 4H), 4.03 (q, 2H), 5.64 (s, 2H), 6.67-6.80 (m, 2H), 6.87 (br. s., 2H), 7.22 (t, 1H), 7.35-7.51 (m, 1H), 7.73 (d, 1H), 8.09 (s, 1H), 8.59 (d, 1H).

The following intermediates were prepared according to the same procedure from the indicated starting materials (SM=starting material):

| | | | |
|---|---|---|---|
| 1-3-2 SM = 1-1-1, 1-4-2 | 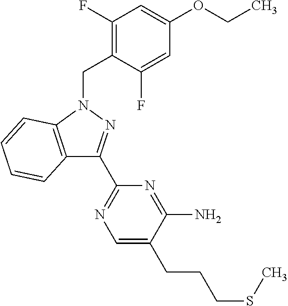 [SMILES: CCOc1cc(F)c(Cn2nc(-c3ncc(CCCSC)c(N)n3)c3ccccc32)c(F)c1] | 2-[1-(4-ethoxy- 2,6- difluorobenzyl)- 1H-indazol-3-yl]- 5-[3- (methylsulfanyl) propyl]pyrimidin-4- amine | ¹H NMR (300 MHz, DMSO-d₆) δ [ppm] = 1.28 (t, 3H), 1.69- 1.84 (m, 2H), 2.03 (s, 3H), 2.51-2.58 (m, 2H), 3.38- 3.53 (m, 2H), 4.03 (q, 2H), 5.64 (s, 2H), 6.68-6.80 (m, 2H), 6.84 (br. s., 2H), 7.21 (t, 1H), 7.36-7.49 (m, 1H), 7.73 (d, 1H), 8.05 (s, 1H), 8.59 (d, 1H). |
| 1-3-3 SM = 1-1-1, 1-4-3 | 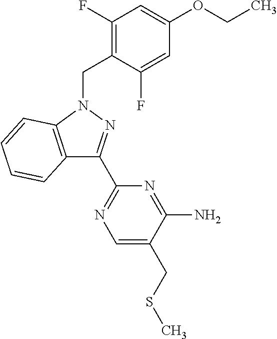 [SMILES: CCOc1cc(F)c(Cn2nc(-c3ncc(CSC)c(N)n3)c3ccccc32)c(F)c1] | 2-[1-(4-ethoxy- 2,6- difluorobenzyl)- 1H-indazol-3-yl]- 5- [(methylsulfanyl) methyl]pyrimidin- 4-amine | ¹H NMR (400 MHz, DMSO-d₆) δ [ppm] = 1.28 (t, 3H), 1.95 (s, 3H), 3.60 (s, 2H), 4.03 (q, 2H), 5.65 (s, 2H), 6.69-6.80 (m, 2H), 6.89-7.06 (m, 2H), 7.19- 7.28 (m, 1H), 7.39-7.49 (m, 1H), 7.74 (d, 1H), 8.09 (s, 1H), 8.59 (d, 1H). |
| 1-3-4 SM = 1-1-1, 1-4-5 | 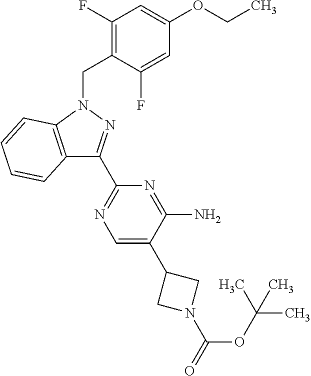 [SMILES: CCOc1cc(F)c(Cn2nc(-c3ncc(C4CN(C(=O)OC(C)(C)C)C4)c(N)n3)c3ccccc32)c(F)c1] | tert-butyl 3-{4- amino-2-[1-(4- ethoxy-2,6- difluorobenzyl)- 1H-indazol-3- yl]pyrimidin-5- yl}azetidine-1- carboxylate | ¹H NMR (300 MHz, DMSO-d₆) δ [ppm] = 1.28 (t, 3 H) 1.39 (s, 9 H) 3.71-3.80 (m, 1 H) 3.92 (t, 2 H) 3.99-4.07 (m, 3 H) 4.26 (t, 2 H) 5.65 (s, 2 H) 6.75 (d, 3 H) 7.23 (t, 1 H) 7.41- 7.48 (m, 1 H) 7.74 (d, 1 H) 8.29 (s, 1 H) 8.59 (d, 1 H). |
| 1-3-5 SM = 1-1-1, 1-4-7 | 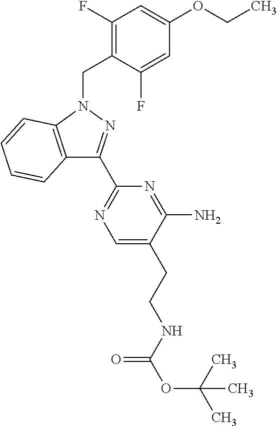 [SMILES: CCOc1cc(F)c(Cn2nc(-c3ncc(CCNC(=O)OC(C)(C)C)c(N)n3)c3ccccc32)c(F)c1] | tert-butyl (2-{4- amino-2-[1-(4- ethoxy-2,6- difluorobenzyl)- 1H-indazol-3- yl]pyrimidin-5- yl}ethyl)carbamate | ¹H NMR (300 MHz, DMSO-d₆) δ [ppm] = 1.28 (t, 3H), 1.35 (s, 9H), 2.52-2.59 (m, 2H), 3.11 (q, 2H), 4.03 (q, 2H), 5.64 (s, 2H), 6.70-6.78 (m, 2H), 6.80- 6.92 (m, 3H), 7.21 (t, 1H), 7.43 (t, 1H), 7.73 (d, 1H), 8.00 (s, 1H), 8.57 (d, 1H). |
| 1-3-6 SM = 1-1-1 | 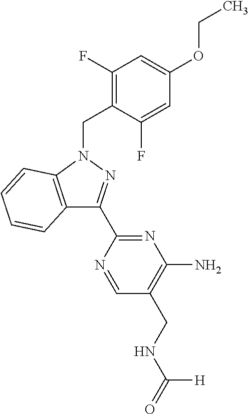 [SMILES: CCOc1cc(F)c(Cn2nc(-c3ncc(CNC=O)c(N)n3)c3ccccc32)c(F)c1] | N-({4-amino-2-[1- (4-ethoxy-2,6- difluorobenzyl)- 1H-indazol-3- yl]pyrimidin-5- yl}methyl)formamide | ¹H NMR (400 MHz, DMSO-d₆) δ [ppm] = 1.28 (t, 3H), 4.03 (q, 2H), 4.16 (d, 2H), 5.66 (s, 2H), 6.68-6.79 (m, 2H), 6.96 (br. s., 2H), 7.15-7.28 (m, 1H), 7.39-7.49 (m, 1H), 7.74 (d, 1H), 8.11-8.16 (m, 2H), 8.48 (t, 1H), 8.58 (d, 1H). |
| 1-3-7 SM = 1-1-1, 1-4-8 | 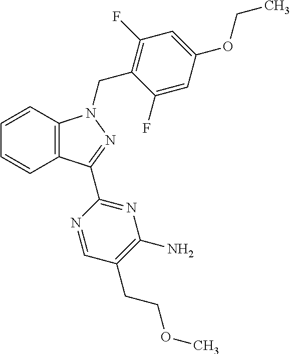 [SMILES: CCOc1cc(F)c(Cn2nc(-c3ncc(CCOC)c(N)n3)c3ccccc32)c(F)c1] | 2-[1-(4-ethoxy- 2,6- difluorobenzyl)- 1H-indazol-3-yl]- 5-(2- methoxyethyl) pyrimidin-4-amine | ¹H NMR (300 MHz, DMSO-d₆) δ [ppm] = 1.28 (t, 3H), 2.68 (t, 2H), 3.25 (s, 3H), 3.46-3.56 (m, 2H), 4.02 (q, 2H), 5.64 (s, 2H), 6.65-6.80 (m, 2H), 6.85 (br. s., 2H), 7.23 (t, 1H), 7.44 (t, 1H), 7.73 (d, 1H), 8.05 (s, 1H), 8.58 (d, 1H). |
| 1-3-8 SM = 1-1-1, 1-4-9 | 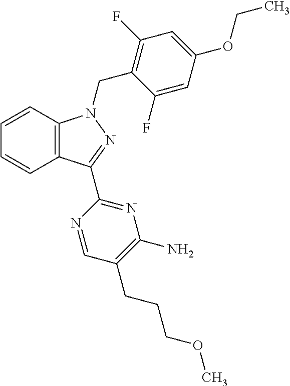 [SMILES: CCOc1cc(F)c(Cn2nc(-c3ncc(CCCOC)c(N)n3)c3ccccc32)c(F)c1] | 2-[1-(4-ethoxy- 2,6- difluorobenzyl)- 1H-indazol-3-yl]- 5-(3- methoxypropyl) pyrimidin-4-amine | ¹H NMR (300 MHz, DMSO-d₆) δ [ppm] = 1.28 (t, 3H), 1.67- 1.83 (m, 2H), 2.38-2.47 (m, 2H), 3.23 (s, 3H), 3.45-3.55 (m, 2H), 4.03 (q, 2H), 5.64 (s, 2H), 6.66-6.91 (m, 4H), 7.21 (t, 1H), 7.37-7.51 (m, 1H), 7.74 (d, 1H), 8.03 (s, 1H), 8.59 (d, 1H). |
| 1-3-9 SM = 1-1-1, 1-4-10 | 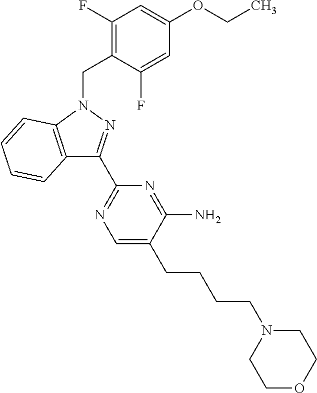 [SMILES: CCOc1cc(F)c(Cn2nc(-c3ncc(CCCCN4CCOCC4)c(N)n3)c3ccccc32)c(F)c1] | 2-[1-(4-ethoxy- 2,6- difluorobenzyl)- 1H-indazol-3-yl]- 5-[4-(morpholin- 4- yl)butyl]pyrimidin- 4-amine | ¹H NMR (300 MHz, DMSO-d₆) δ [ppm] = 1.28 (t, 3 H) 2.25 (m, 8 H) 2.42 (t, 2 H) 3.55 (m, 6 H) 4.02 (m, 2 H) 5.63 (s, 2 H) 6.75 (m, 2 H) 6.87 (br. s., 2 H) 7.21 (t, 1 H) 7.43 (m, 1 H) 7.74 (d, 1 H) 8.04 (s, 1 H) 8.59 (d, 1 H). |
| 1-3-10 SM = 1-1-1, 1-4-12 | 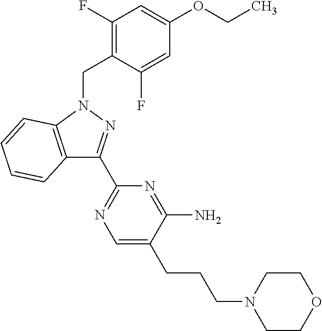 [SMILES: CCOc1cc(F)c(Cn2nc(-c3ncc(CCCN4CCOCC4)c(N)n3)c3ccccc32)c(F)c1] | 2-[1-(4-ethoxy- 2,6- difluorobenzyl)- 1H-indazol-3-yl]- 5-[3-(morpholin- 4- yl)propyl]pyrimidin- 4-amine | ¹H NMR (400 MHz, DMSO-d₆) δ [ppm] = 1.28 (t, 3 H) 1.68 (m, 2 H) 2.27 (t, 2 H) 2.33 (br. s., 4 H) 2.44 (t, 2 H) 3.57 (t, 4 H) 4.04 (m, 2 H) 5.61 (m, 2 H) 6.74 (d, 2 H) 6.89 (br. s., 2 H) 7.21 (t, 1 H) 7.43 (t, 1 H) 7.73 (d, 1 H) 8.06 (s, 1 H) 8.58 (d, 1 H). |
| 1-3-11 SM = 1-1-1, 1-4-13 | 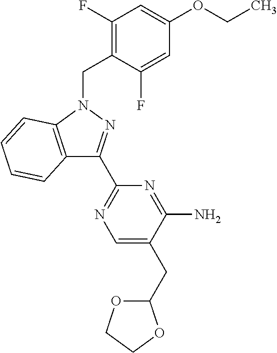 [SMILES: CCOc1cc(F)c(Cn2nc(-c3ncc(CC4OCCO4)c(N)n3)c3ccccc32)c(F)c1] | 5-(1,3-dioxolan- 2-ylmethyl)-2-[1- (4-ethoxy-2,6- difluorobenzyl)- 1H-indazol-3- yl]pyrimidin-4- amine | ¹H NMR (300 MHz, DMSO-d₆) δ [ppm] = 1.28 (t, 3H) 2.77 (m, 2 H) 3.79 (m, 2 H) 3.89 (m, 2 H) 4.03 (q, 2 H) 5.03 (t, 1 H) 5.64 (s, 2 H) 6.73 (m, 2 H) 6.82 (br. s., 2 H) 7.22 (t, 1 H) 7.43 (m, 1 H) 7.73 (d, 1 H) 8.09 (s, 1 H) 8.58 (d, 1 H) |
| 1-3-12 SM = 1-1-1, 1-4-14 | 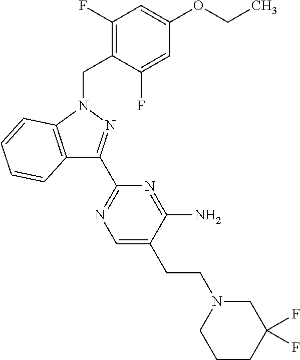 [SMILES: CCOc1cc(F)c(Cn2nc(-c3ncc(CCN4CCCC(F)(F)C4)c(N)n3)c3ccccc32)c(F)c1] | 5-[2-(3,3- difluoropiperidin- 1-yl)ethyl]-2-[1- (4-ethoxy-2,6- difluorobenzyl)- 1H-indazol-3- yl]pyrimidin-4- amine | LC-MS (Method 6): retention time: 1.43 min MS ES+: 529 [M + H]⁺ |
| 1-3-13 SM = 1-1-1, 1-4-15 | 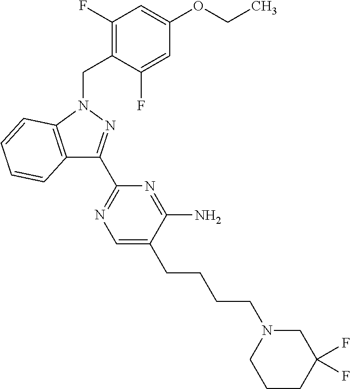 [SMILES: CCOc1cc(F)c(Cn2nc(-c3ncc(CCCCN4CCCC(F)(F)C4)c(N)n3)c3ccccc32)c(F)c1] | 5-[4-(3,3- difluoropiperidin- 1-yl)butyl]-2-[1- (4-ethoxy-2,6- difluorobenzyl)- 1H-indazol-3- yl]pyrimidin-4- amine | ¹H NMR (300 MHz, DMSO-d₆) δ [ppm] = 1.28 (t, 3H), 1.49 (br. s., 4H), 1.55-1.68 (m, 2H), 1.71-1.95 (m, 2H), 2.33- 2.46 (m, 6H), 2.53-2.63 (m, 2H), 4.03 (q, 2H), 5.63 (s, 2H), 6.67-6.89 (m, 4H), 7.21 (t, 1H), 7.37-7.51 (m, 1H), 7.73 (d, 1H), 8.04 (s, 1H), 8.59 (d, 1H). |
| 1-3-14 SM = 1-1-1, 1-4-16 | 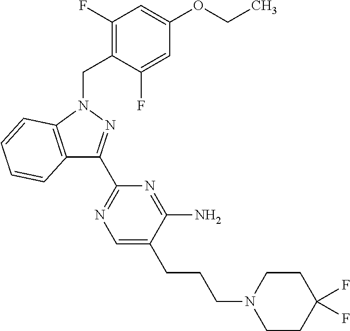 [SMILES: CCOc1cc(F)c(Cn2nc(-c3ncc(CCCN4CCC(F)(F)CC4)c(N)n3)c3ccccc32)c(F)c1] | 5-[3-(4,4- difluoropiperidin- 1-yl)propyl]-2-[1- (4-ethoxy-2,6- difluorobenzyl)- 1H-indazol-3- yl]pyrimidin-4- amine | ¹H NMR (300 MHz, DMSO-d₆) δ [ppm] = 1.27 (m, 3 H) 1.69 (quin, 2 H) 1.94 (m, 6 H) 2.35 (t, 2 H) 2.43 (m, 4 H) 5.64 (s, 2 H) 6.77 (m, 2 H) 6.89 (br. s., 2 H) 7.21 (t, 1 H) 7.43 (m, 1 H) 7.74 (d, 1 H) 8.06 (s, 1 H) 8.58 (d, 1 H). |
| 1-3-15 SM = 1-1-1, 1-4-17 | 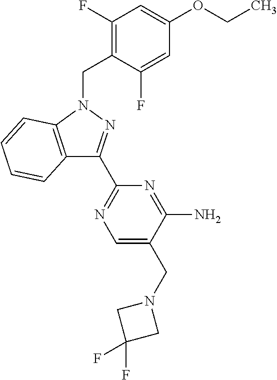 [SMILES: CCOc1cc(F)c(Cn2nc(-c3ncc(CN4CC(F)(F)C4)c(N)n3)c3ccccc32)c(F)c1] | 5-[(3,3- difluoroazetidin- 1-yl)methyl]-2-[1- (4-ethoxy-2,6- difluorobenzyl)- 1H-indazol-3- yl]pyrimidin-4- amine | ¹H NMR (600 MHz, DMSO-d₆) δ [ppm] = 1.28 (t, 3H), 3.56- 3.65 (m, 6H), 4.03 (q, 2H), 5.64 (s, 2H), 6.64-6.80 (m, 2H), 6.84-7.02 (m, 1H), 7.17- 7.27 (m, 1H), 7.44 (ddd, 1H), 7.74 (d, 1H), 8.14 (s, 1H), 8.58 (d, 1H). |
| 1-3-16 SM = 1-1-1, 1-4-18 | 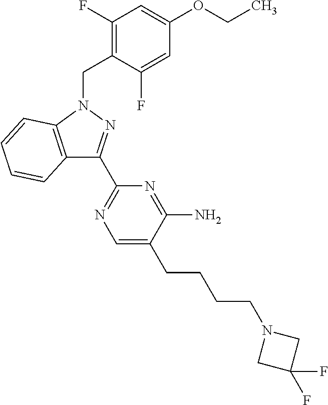 [SMILES: CCOc1cc(F)c(Cn2nc(-c3ncc(CCCCN4CC(F)(F)C4)c(N)n3)c3ccccc32)c(F)c1] | 5-[4-(3,3- difluoroazetidin- 1-yl)butyl]-2-[1- (4-ethoxy-2,6- difluorobenzyl)- 1H-indazol-3- yl]pyrimidin-4- amine | LC-MS (Method 5): retention time: 1.41 min MS ES+: 529 [M + H]⁺ |
| 1-3-17 SM = 1-1-1, 1-4-19 | 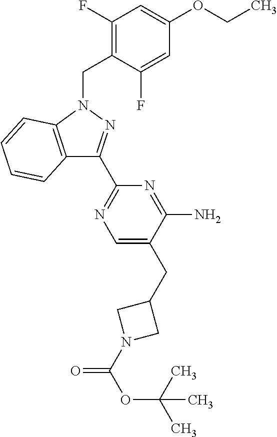 [SMILES: CCOc1cc(F)c(Cn2nc(-c3ncc(CC4CN(C(=O)OC(C)(C)C)C4)c(N)n3)c3ccccc32)c(F)c1] | tert-butyl 3-({4- amino-2-[1-(4- ethoxy-2,6- difluorobenzyl)- 1H-indazol-3- yl]pyrimidin-5- yl}methyl)azetidine- 1-carboxylate | ¹H NMR (400 MHz, DMSO-d₆) δ [ppm] = 1.28 (t, 3H), 1.36 (s, 9H), 2.70 (d, 2H), 2.82 (br. s., 1H), 3.44-3.60 (m, 2H), 3.76- 3.95 (m, 2H), 4.03 (q, 2H), 5.64 (s, 2H), 6.74 (d, 2H), 6.91 (br. s., 2H), 7.08-7.29 (m, 1H), 7.38-7.48 (m, 1H), 7.73 (d, 1H), 8.07 (s, 1H), 8.58 (d, 1H). |
| 1-3-18 SM = 1-1-1, 1-4-20 | 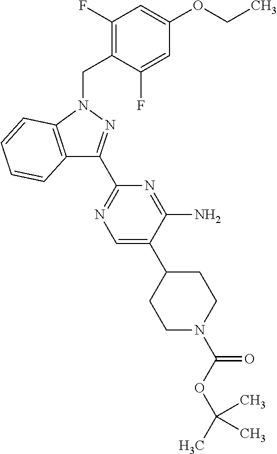 [SMILES: CCOc1cc(F)c(Cn2nc(-c3ncc(C4CCN(C(=O)OC(C)(C)C)CC4)c(N)n3)c3ccccc32)c(F)c1] | tert-butyl 4-{4- amino-2-[1-(4- ethoxy-2,6- difluorobenzyl)- 1H-indazol-3- yl]pyrimidin-5- yl}piperidine-1- carboxylate | ¹H-NMR (300 MHz, DMSO-d₆): δ [ppm] = 1.28 (t, 3H), 1.34- 1.52 (m, 11H), 1.80 (d, 2H), 2.63-2.92 (m, 3H), 3.93- 4.15 (m, 4H), 5.64 (s, 2H), 6.75 (m, 2H), 6.95 (br. s., 2H), 7.21 (t, 1H), 7.43 (t, 1H), 7.74 (d, 1H), 8.09 (s, 1H), 8.58 (d, 1H). |
| 1-3-19 SM = 1-1-1, 1-4-21 | 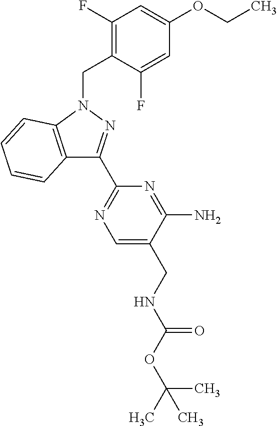 [SMILES: CCOc1cc(F)c(Cn2nc(-c3ncc(CNC(=O)OC(C)(C)C)c(N)n3)c3ccccc32)c(F)c1] | tert-butyl ({4- amino-2-[1-(4- ethoxy-2,6- difluorobenzyl)- 1H-indazol-3- yl]pyrimidin-5- yl}methyl)carbamate | ¹H-NMR (400 MHz, DMSO-d₆): δ [ppm] = 1.31 (t, 3H), 1.38- 1.45 (m, 9H), 3.95-4.02 (m, 2H), 4.05 (q, 2H), 5.67 (s, 2H), 6.70-6.83 (m, 2H), 6.83- 7.05 (m, 2H), 7.25 (t, 1H), 7.36 (t, 1H), 7.47 (ddd, 1H), 7.77 (d, 1H), 8.08 (s, 1H), 8.61 (d, 1H). |
| 1-3-20 SM = 1-1-1, 1-4-22 | 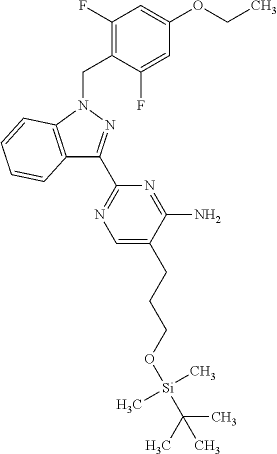 [SMILES: CCOc1cc(F)c(Cn2nc(-c3ncc(CCCO[Si](C)(C)C(C)(C)C)c(N)n3)c3ccccc32)c(F)c1] | 5-(3-{[tert- butyl(dimethyl)silyl] oxy}propyl)-2- [1-(4-ethoxy-2,6- difluorobenzyl)- 1H-indazol-3- yl]pyrimidin-4- amine | ¹H-NMR (400 MHz, DMSO-d₆): δ [ppm] = −0.01-0.09 (m, 6H), 0.88 (s, 9H), 1.30 (t, 3H), 1.64- 1.80 (m, 2H), 2.46-2.49 (m, 2H), 3.64 (t, 2H), 4.04 (q, 2H), 5.67 (s, 2H), 6.77 (d, 2H), 6.86- 7.17 (m, 2H), 7.18-7.34 (m, 1H), 7.39-7.54 (m, 1H), 7.77 (d, 1H), 8.03 (s, 1H), 8.60 (d, 1H). |
| 1-3-21 SM = 1-1-1, 1-4-23 | 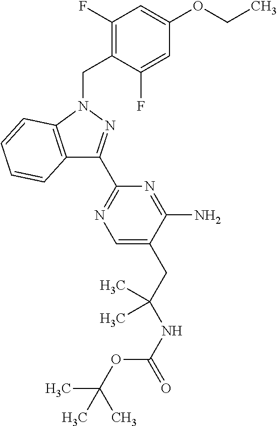 [SMILES: CCOc1cc(F)c(Cn2nc(-c3ncc(CC(C)(C)NC(=O)OC(C)(C)C)c(N)n3)c3ccccc32)c(F)c1] | tert-butyl (1-{4- amino-2-[1-(4- ethoxy-2,6- difluorobenzyl)- 1H-indazol-3- yl]pyrimidin-5-yl}- 2-methylpropan- 2-yl)carbamate | LC-MS (Method 6): retention time: 1.50 min MS ES+: 553 [M + H]⁺ |
| 1-3-22 SM = 1-1-3, 1-4-20 | 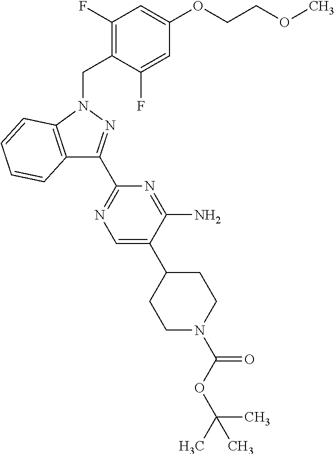 [SMILES: COCCOc1cc(F)c(Cn2nc(-c3ncc(C4CCN(C(=O)OC(C)(C)C)CC4)c(N)n3)c3ccccc32)c(F)c1] | tert-butyl 4-(4- amino-2-{1-[2,6- difluoro-4-(2- methoxyethoxy) benzyl]-1H- indazol-3- yl}pyrimidin-5- yl)piperidine-1- carboxylate | ¹H-NMR (500 MHz, DMSO- d₆): δ [ppm] = 1.42 (s, 9H), 1.44 (m, 2H), 1.81 (br. d., 2H), 2.71 (m, 1H), 2.83 (m, 2H), 3.27 (s, 3H), 3.61 (m, 2H), 4.09 (m, 2H), 4.11 (m, 2H), 5.65 (s, 2H), 6.80 (m, 2H), 6.96 (br. s., 2H), 7.22 (dd, 1H), 7.44 (dd, 1H), 7.74 (d, 1H), 8.10 (s, 1H), 8.59 (d, 1H). |

### Intermediate 1-4-1

### Preparation of (2Z)-2-[(dimethylamino)methylene]-4-(methylsulfanyl)butanenitrile

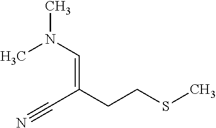

5.9 g of 4-(methylsulfanyl)butanenitrile 1-5-1 (51.7 mmol, 1.0 eq.) were dissolved in 99 mL DMF, 13.5 g of 1-tert-butoxy-N,N,N′,N′-tetramethylmethanediamine (77.5 mmol, 1.5 eq.) were added and the mixture was stirred at 140° C. over night. 13.5 g of 1-tert-butoxy-N,N,N′,N′-tetramethylmethanediamine (77.5 mmol, 1.5 eq.) were added a second time and the mixture was stirred at 140° C. over night again. Stirring was continued at 110° C. for further 24 hours. The reaction mixture was concentrated under reduced pressure under reduced pressure and the crude product was purified by flash chromatography to provide the target compound in 95% purity: 6.9 g, 5.8 mmol, 74%.,

¹H-NMR (500 MHz, chloroform-d): δ [ppm]=2.13 (s, 3H), 2.28-2.36 (m, 2H), 2.63 (t, 2H), 3.05 (s, 6H), 6.27 (s, 1H).

The following intermediates were prepared according to the same procedure from the indicated starting materials (SM=starting material):

| | | | |
|---|---|---|---|
| 1-4-2 SM = 1-5-2 | 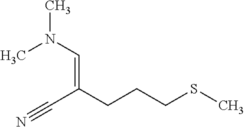 | (2Z)-2- [(dimethylamino) methylene]-5- (methylsulfanyl) pentanenitrile | ¹H-NMR (300 MHz, chloroform- d): δ [ppm] =. 1.73-1.91 (m, 2H), 2.12 (s, 3H), 2.19 (t, 2H), 2.54 (t, 2H), 3.06 (s, 6H), 6.27 (s, 1H). |
| 1-4-3 Commercial available | 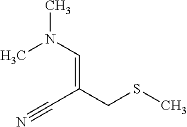 | (2E)-3- (dimethylamino)- 2- [(methylsulfanyl) methyl]acrylonitrile | ¹H NMR (400 MHz, DMSO-d6) δ [ppm] = 1.98 (s, 3H), 3.01 (s, 6H), 3.20 (s, 2H), 6.73 (s, 1H) |
| 1-4-4 Commercial available | 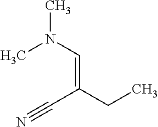 | (2Z)-2- [(dimethylamino) methylene]butane- nitrile | ¹H-NMR (400 MHz, chloroform- d) δ [ppm] =. 1.11 (t, 3H), 2.10 (q, 2H), 3.05 (s, 6H), 6.24 (s, 1H). |
| 1-4-5 SM = 1-7-1 | 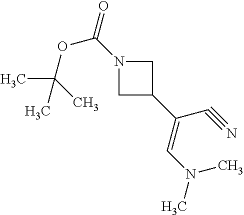 [SMILES: CN(C)/C=C(\C#N)C1CN(C(=O)OC(C)(C)C)C1] | tert-butyl 3-[(Z)- 1-cyano-2- (dimethylamino) vinyl]azetidine-1- carboxylate | ¹H-NMR (400 MHz, chloroform- d): δ [ppm] =. 1.46 (s, 9H) 3.04- 3.21 (m, 7 H) 3.91-3.99 (m, 2 H) 4.01-4.16 (m, 2 H) 6.31 (s, 1 H). |
| 1-4-6 SM = 1-9-1 | 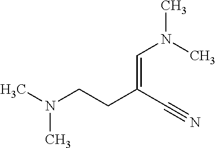 | (2Z)-4- (dimethylamino)- 2- [(dimethylamino) methylene]butane- nitrile | ¹H-NMR (400 MHz, chloroform- d): δ [ppm] = 2.18-2.26 (m, 2H), 2.29 (s, 6H), 2.41-2.49 (m, 2H), 3.06 (s, 6H), 6.28 (s, 1H). |
| 1-4-7 Commercial available SM | 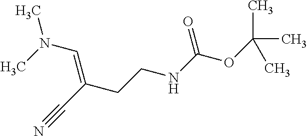 [SMILES: CN(C)/C=C(\C#N)CCNC(=O)OC(C)(C)C] | tert-butyl [(3Z)-3- cyano-4- (dimethylamino) but-3-en-1- yl]carbamate | ¹H-NMR (300 MHz, chloroform- d): δ [ppm] = 1.47 (s, 9H), 2.25 (t, 2H), 3.07 (s, 6H), 3.23 (q, 2H), 4.64-4.80 (m, 1H), 6.27 (s, 1H). |
| 1-4-8 SM: Commercial available | 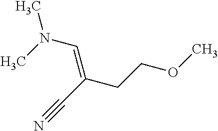 | (2Z)-2- [(dimethylamino) methylene]-4- methoxybutane- nitrile | Used without further purification. |
| 1-4-9 SM = 1-5-3 | 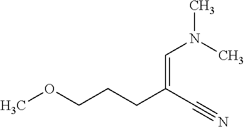 | (2Z)-2- [(dimethylamino) methylene]-5- methoxypentane nitrile | Used without further purification. |
| 1-4-10 SM = 1-9-2 | 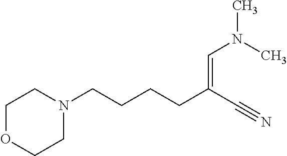 [SMILES: CN(C)/C=C(\C#N)CCCCN1CCOCC1] | (2Z)-2- [(dimethylamino) methylene]-6- (morpholin-4- yl)hexanenitrile | ¹H-NMR (300 MHz, chloroform- d): δ [ppm] = 1.53 (m, 4H) 2.07 (m, 2 H) 2.37 (m, 2 H) 2.46 (br. s., 4 H) 3.03 (m, 6 H) 3.74 (m, 4 H) 6.22 (s, 1 H). |
| 1-4-11 SM = 1-9-3 | 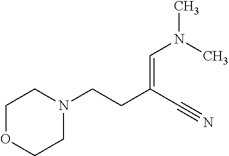 | (2Z)-2- [(dimethylamino) methylene]-4- (morpholin-4- yl)butanenitrile | LC-MS (Method 5): retention time: 0.75 min MS ES+: 211 [M + H]⁺ |
| 1-4-12 SM = 1-9-4 | 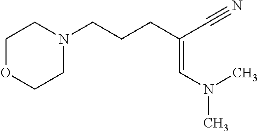 [SMILES: CN(C)/C=C(\C#N)CCCN1CCOCC1] | (2Z)-2- [(dimethylamino) methylene]-5- (morpholin-4- yl)pentanenitrile | LC-MS (Method 5): retention time: 0.84 min MS ES+: 224 [M + H]⁺ |
| 1-4-13 SM = 1-10-1 | 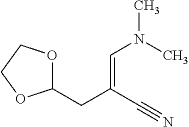 | (2Z)-3- (dimethylamino)- 2-(1,3-dioxolan- 2- ylmethyl)acrylonitrile | ¹H-NMR (400 MHz, chloroform- d): δ [ppm] = 2.37 (d, 2 H) 3.09 (s, 6 H) 3.90 (m, 2 H) 4.02 (m, 2 H) 4.98 (t, 1 H) 6.33 (s, 1 H). |
| 1-4-14 SM = 1-9-5 | 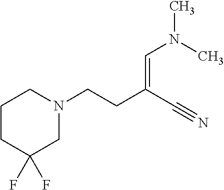 [SMILES: CN(C)/C=C(\C#N)CCN1CCCC(F)(F)C1] | (2Z)-4-(3,3- difluoropiperidin- 1-yl)-2- [(dimethylamino) methylene]butane- nitrile | ¹H-NMR (400 MHz, chloroform- d): δ [ppm] = 1.77 (m, 2H), 1.89 (m, 2H), 2.23 (t, 2H), 2.48 (m, 2H), 2.56 (t, 2H), 2.67 (t, 2H), 3.05 (s, 6H), 6.30 (s, 1H). |
| 1-4-15 SM = 1-9-6 | 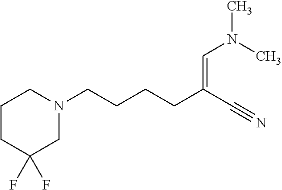 [SMILES: CN(C)/C=C(\C#N)CCCCN1CCCC(F)(F)C1] | (2Z)-6-(3,3- difluoropiperidin- 1-yl)-2- [(dimethylamino) methylene]hexane- nitrile | LC-MS (Method 6): retention time: 1.18 min MS ES+: 272 [M + H]⁺ |
| 1-4-16 SM = 1-9-7 | 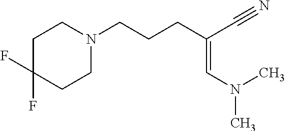 [SMILES: CN(C)/C=C(\C#N)CCCN1CCC(F)(F)CC1] | (2Z)-5-(4,4- difluoropiperidin- 1-yl)-2- [(dimethylamino) methylene]pentane- nitrile | LC-MS (Method 5): retention time: 1.11 min MS ES+: 258 [M + H]⁺ |
| 1-4-17 SM = 1-11-1 | 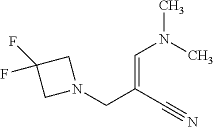 | (2Z)-2-[(3,3- difluoroazetidin- 1-yl)methyl]-3- (dimethylamino) acrylonitrile | ¹H-NMR (400 MHz, chloroform- d): δ [ppm] = 3.11 (s, 6H), 3.18 (s, 2H), 3.62 (t, 4H), 6.42 (s, 1H). |
| 1-4-18 SM = 1-9-8 | 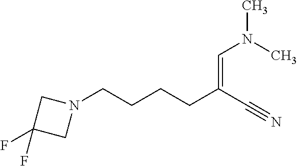 [SMILES: CN(C)/C=C(\C#N)CCCCN1CC(F)(F)C1] | (2Z)-6-(3,3- difluoroazetidin- 1-yl)-2- [(dimethylamino) methylene]hexane- nitrile | LC-MS (Method 5): retention time: 1.05 min MS ES+: 245 [M + H]⁺ |
| 1-4-19 SM = 1-7-2 | 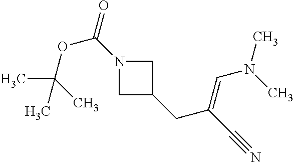 [SMILES: CN(C)/C=C(\C#N)CC1CN(C(=O)OC(C)(C)C)C1] | tert-butyl 3-[(2Z)- 2-cyano-3- (dimethylamino) prop-2-en-1- yl]azetidine-1- carboxylate | ¹H-NMR (300 MHz, chloroform- d): δ [ppm] = 1.45 (s, 9H), 2.32 (d, 2H), 2.60-2.79 (m, 1H), 3.07 (s, 6H), 3.55 (dd, 2H), 4.02 (t, 2H), 6.28 (s, 1H). |
| 1-4-20 | 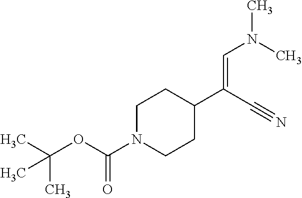 [SMILES: CN(C)/C=C(\C#N)C1CCN(C(=O)OC(C)(C)C)CC1] | tert-butyl 4-[(Z)- 1-cyano-2- (dimethylamino) ethenyl]piperidine- 1-carboxylate | LC-MS (Method 5): retention time: 1.19 min MS ES+: 280 [M + H]⁺ |
| 1-4-21 | 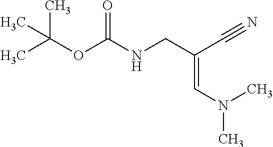 [SMILES: CN(C)/C=C(\C#N)CNC(=O)OC(C)(C)C] | tert-butyl [(2Z)-2- cyano-3- (dimethylamino) prop-2-en-1- yl]carbamate | ¹H-NMR (400 MHz, CHLOROFORM-d): δ [ppm] = 1.46 (s, 9H), 3.09 (s, 6H), 3.72 (d, 2H), 4.66-5.05 (m, 1H), 6.54 (br. s., 1H). |
| 1-4-22 SM = 1-16-1 | 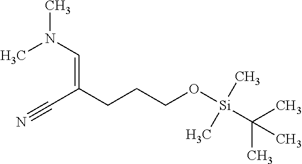 [SMILES: CN(C)/C=C(\C#N)CCCO[Si](C)(C)C(C)(C)C] | (2Z)-5-{[tert- butyl(dimethyl) silyl]oxy}-2- [(dimethylamino) methylidene] pentanenitrile | ¹H-NMR (400 MHz, CHLOROFORM-d): δ [ppm] = 0.07 (s, 6H), 0.91 (s, 9H), 1.66- 1.73 (m, 2H), 2.15 (t, 2H), 3.05 (s, 6H), 3.65 (t, 2H), 6.25 (s, 1H). |
| 1-4-23 SM = 1-16-1 16 h at 110° C. | 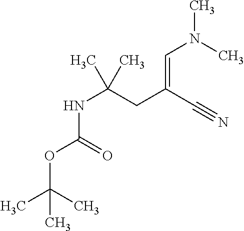 [SMILES: CN(C)/C=C(\C#N)CC(C)(C)NC(=O)OC(C)(C)C] | tert-butyl [(4Z)-4- cyano-5- (dimethylamino)- 2-methylpent-4- en-2- yl]carbamate | ¹H-NMR (400 MHz, CHLOROFORM-d): δ [ppm] = 1.30 (s, 6H), 1.45 (s, 9H), 2.45 (s, 2H), 3.07 (s, 6H), 4.49 (br. s., 1H), 6.27 (s, 1H). |
| 1-4-24 Over night, at 100° C., without solvent | 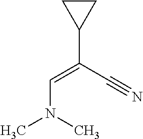 | (2Z)-2- cyclopropyl-3- (dimethylamino) prop-2-enenitrile | ¹H-NMR (300 MHz, DMSO-d₆): δ [ppm] = 0.26-0.33 (m, 2H), 0.51-0.59 (m, 2H), 1.32- 1.43 (m, 1H), 2.93 (s, 6H), 6.63 (s, 1H). |

### Intermediate 1-5-1

### Preparation of 4-(methylsulfanyl)butanenitrile

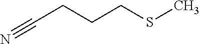

8.9 g of sodium thiomethylate (126 mmol, 1.25 eq.) were dissolved in 510 mL methanol. To this solution 10 mL of 4-bromobutyronitrile (101 mmol, 1.0 eq.) were added dropwise.

The reaction mixture was stirred over night at rt and was concentrated under reduced pressure. The residue was dissolved in DCM and was washed three times with water. The organic layer was dried using a waterresistant filter and the filtrate was concentrated under reduced pressure. The crude product was used without further purification: 99% purity, 11 g, 94 mmol, 93%.

¹H-NMR (300 MHz, chloroform-d): δ [ppm]=1.98 (quin, 2H), 2.13 (s, 3H), 2.54 (t, 2H), 2.66 (t, 2H).

The following intermediates were prepared according to the same procedure from commercial available compounds:

| | | | |
|---|---|---|---|
| 1-5-2 | 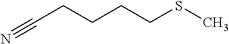 | 5-(methylsulfanyl) pentanenitrile | ¹H-NMR (300 MHz, chloroform- d): δ [ppm] =. 1.71-1.88 (m, 4H), 2.13 (s, 3H), 2.41 (t, 2H), 2.51-2.64 (m, 2H). |
| 1-5-3 | 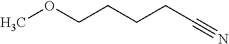 | 5-methoxypentane- nitrile | ¹H NMR (400 MHz, DMSO-d6) δ [ppm] = 1.49-1.64 (m, 4H), 2.43-2.53 (m, 2H), 3.21 (s, 3H), 3.30-3.36 (m, 2H). |

### Intermediate 1-6-1

### Preparation of 2-[1-(4-ethoxy-2,6-difluorobenzyl)-1H-indazol-3-yl]-5-methylpyrimidin-4-amine

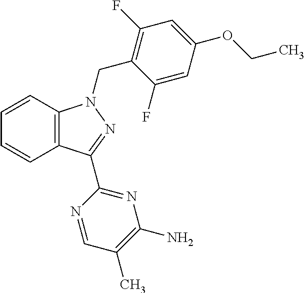 [SMILES: CCOc1cc(F)c(Cn2nc(-c3ncc(C)c(N)n3)c3ccccc32)c(F)c1]

500 mg of 1-(4-ethoxy-2,6-difluorobenzyl)-1H-indazole-3-carboximidamide hydrochloride 1:1 1-1-1 (1.36 mmol, 1.0 eq.), 225 mg of 2-(piperidinomethyl)acrylonitrile (1.50 mmol, 1.1 eq.) and 7.4 mg of sodium methylate (0.14 mmol, 0.1 eq.) were dissolved in 1.4 mL ethanol. The reaction mixture was stirred over night under reflux. Again 306 mg of 2-(piperidinomethyl)acrylonitrile (2.05 mmol, 1.5 eq.) and 7.4 mg of sodium methylate (0.14 mmol, 0.1 eq.) were added and the reaction mixture was stirred over night under reflux. The reaction mixture was diluted by adding water and ethanol. The precipitate was filtered off and was washed with water and dissolved with DCM. The aqueous layer was extracted with DCM three times. The combined organic layers were washed with water and brine and were dried using a waterresistant filter and the filtrate was concentrated under reduced pressure. The crude product was purified by HPLC to provide the target compound in 93% purity: 42 mg, 0.11 mmol, 8%.

LC-MS: retention time: 0.94 min; MS ES+: 397.1 [M+H]⁺.

### Intermediate 1-7-1

### Preparation of tert-butyl 3-(cyanomethyl)azetidine-1-carboxylate

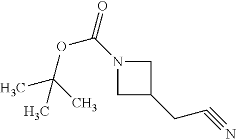

2.60 g of tert-butyl 3-(cyanomethylidene)azetidine-1-carboxylate 1-8-1 (13.9 mmol, 1.0 eq.) was dissolved in 130 mL methanol and treated with 513 mg palladium 10% on carbon and 50% water (0.48 mmol, 0.036 eq.). It was stirred for two hours at room temperature under hydrogen atmosphere. The catalyst was filtered off and the filtrate was concentrated under reduced pressure to provide 2.63 g of the target compound. 13.3 mmol, 98%.

¹H-NMR (400 MHz, chloroform-d): δ [ppm]=. 1.47 (s, 9H) 2.66 (d, J=7.07 Hz, 2H) 2.83-2.93 (m, 1H) 3.71 (dd, J=9.09, 5.05 Hz, 2H) 4.15 (t, J=8.59 Hz, 2H).

The following intermediate was prepared according to the same procedure from the indicated starting materials (SM=starting material):

| | | | |
|---|---|---|---|
| 1-7-2 | 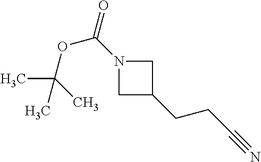 | tert-butyl 3-(2- cyanoethyl) azetidine-1- carboxylate | ¹H-NMR (300 MHz, chloroform- d): δ [ppm] = 1.46 (s, 9H), 1.92-2.06 (m, 2H), 2.36 (t, 2H), 2.56-2.76 (m, 1H), 3.61 (dd, 2H), 4.10 (t, 2H). |

### Intermediate 1-8-1

### Preparation of tert-butyl 3-(cyanomethylene)azetidine-1-carboxylate

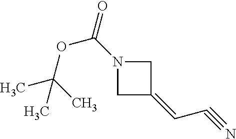

0.81 g sodiumhydride (55% purity, 18.6 mmol, 1.1 eq.) was suspended in 96 mL THF under argon atmosphere. At 4° C. bath temperature 3.45 g diethyl (cyanomethyl)phosphonate (19.5 mmol, 1.15 eq.) was added drop wise. After 45 minutes at room temperature 2.90 g of the commercial available starting material tert-butyl 3-oxoazetidine-1-carboxylate (16.9 mmol, 1.0 eq.), dissolved in 15 mL THF, were added drop wise. It was stirred at room temperature for 16 hours. The reaction mixture was poured into half concentrated sodium chloride solution and extracted three times with EE, dried over sodium sulfate and was concentrated under reduced pressure. The crude product was purified by flash chromatography to provide 2.40 g of the target compound: 12.36 mmol, 73%.

¹H NMR (400 MHz, DMSO-d₆) δ[ppm]=1.34-1.43 (m, 9H) 4.57 (br. s., 2H) 4.64 (br. s., 2H) 5.79-5.86 (m, 1H).

The following intermediate was prepared according to the same procedure from commercial available compounds:

| | | | |
|---|---|---|---|
| 1-8-2 | 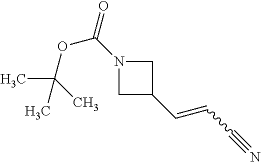 | tert-butyl 3-[2- cyanovinyl]azetidine- 1-carboxylate (E,Z-mixture) | ¹H-NMR (300 MHz, chloroform- d): δ [ppm] = 1.47 (s, 9H), 3.37 + 3.76 (m, 1H), 3.78- 3.84 (t, 2H), 4.18 + 4.27 (t, 2H), 5.41 + 5.44 (dd, 1H), 6.73 + 6.89 (dd, 1H). |

### Intermediate 1-9-1

### Preparation of 4-(dimethylamino)butanenitrile

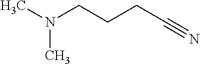

5.51 g of N-methylmethanamine hydrochloride (67.5 mmol, 1.0 eq.), in 150 mL ACN were treated with 14.0 g potassium carbonate (101 mmol, 1.5 eq.) and 1.12 g potassium iodide (6.75 mmol, 0.1 eq.). At 50° C. bath temperature the 4-bromobutanenitrile, dissolved in 50 mL ACN, was added dropwise. It was stirred at this temperature for 16 hours. The reaction mixture was concentrated under reduced pressure and extracted four times with diethyl ether, dried over sodium sulfate and concentrated under reduced pressure. To provide 7.35 g of the target compound in 95% purity: 65.5 mmol, 97%.

¹H-NMR (300 MHz, chloroform-d): δ [ppm]=1.75-1.90 (m, 2H), 2.24 (s, 6H), 2.42 (dt, 4H).

The following intermediates were prepared according to the same procedure from commercial available compounds:

| | | | |
|---|---|---|---|
| 1-9-2 | 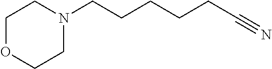 [SMILES: N#CCCCCCN1CCOCC1] | 6-(morpholin-4- yl)hexanenitrile | LC-MS (Method 5): retention time: 0.73 min MS ES+: 183 [M + H]⁺ |
| 1-9-3 | 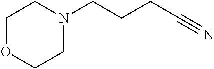 [SMILES: N#CCCCN1CCOCC1] | 4-(morpholin-4- yl)butanenitrile | ¹H-NMR (300 MHz, chloroform- d): δ [ppm] = 1.85 (quin, 2 H) 2.47 (m, 8 H) 3.73 (m, 4 H). |
| 1-9-4 | 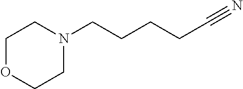 [SMILES: N#CCCCCN1CCOCC1] | 5-(morpholin-4- yl)pentanenitrile | ¹H-NMR (300 MHz, chloroform- d): δ [ppm] = 1.53 (m, 4 H) 1.71 (quin, 2 H) 2.36 (m, 4 H) 2.44 (m, 4 H) 3.73 (m, 4 H). |
| 1-9-5 | 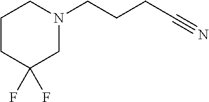 [SMILES: N#CCCCN1CCCC(F)(F)C1] | 4-(3,3- difluoropiperidin- 1-yl)butanenitrile | ¹H-NMR (300 MHz, chloroform- d): δ [ppm] = 1.70-1.99 (m, 6H), 2.47 (m, 4H), 2.53-2.59 (m, 2H), 2.60-2.69 (m, 2H). |
| 1-9-6 | 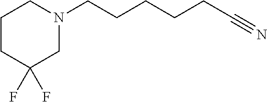 [SMILES: N#CCCCCCN1CCCC(F)(F)C1] | 6-(3,3- difluoropiperidin- 1-yl)hexanenitrile | ¹H-NMR (300 MHz, chloroform- d): δ [ppm] = 1.45-1.59 (m, 4H), 1.67-1.74 (m, 2H), 1.74- 1.82 (m, 2H), 1.84-1.98 (m, 2H), 2.34-2.41 (m, 2H), 2.41- 2.49 (m, 4H), 2.63 (t, 2H). |
| 1-9-7 | 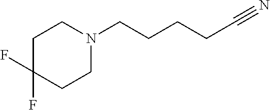 [SMILES: N#CCCCCN1CCC(F)(F)CC1] | 5-(4,4- difluoropiperidin- 1- yl)pentanenitrile | ¹H-NMR (400 MHz, chloroform- d): δ [ppm] = 1.63-1.78 (m, 4H), 2.02 (ddd, 4H), 2.43 (dt, 4H), 2.56 (t, 4H). |
| 1-9-8 | 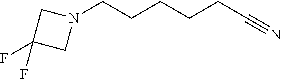 [SMILES: N#CCCCCCN1CC(F)(F)C1] | 6-(3,3- difluoroazetidin- 1-yl)hexanenitrile | ¹H-NMR (300 MHz, chloroform- d): δ [ppm] = 1.38-1.59 (m, 4H), 1.70 (quin, 2H), 2.38 (t, 2H), 2.60 (t, 2H), 3.59 (t, 4H). |

### Intermediate 1-10-1

### Preparation of 3-(1,3-dioxolan-2-yl)propanenitrile

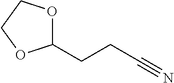

10.0 g of the commercial available 2-(2-bromoethyl)-1,3-dioxolane (55.2 mmol, 1.0 eq.) were dissolved in 25 mL DMSO at room temperature and treated with 3.90 g sodium cyanide (79.5 mmol, 1.4 eq.) for 1 hour at room temperature and 5 hours at 80° C. and over night at room temperature under argon atmosphere. The thick reaction mixture was poured into ice water and stirred for 10 minutes. It was extracted once with diethyl ether and three times with DCM, dried with sodium sulfate and concentrated under reduced pressure to provide 7.42 g of the target compound, 105% which was used without further purification.

¹H-NMR (400 MHz, chloroform-d): δ [ppm]=2.06 (m, 2H) 2.49 (m, 2H) 3.92 (m, 2H) 4.01 (m, 2H) 5.01 (m, 1H).

### Intermediate 1-11-1

### Preparation of 3-(3,3-difluoroazetidin-1-yl)propanenitrile

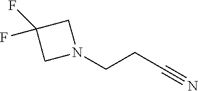

2.65 g of prop-2-enenitrile (50.0 mmol, 1.5 eq.) were dissolved in 16 mL acetonitrile and treated with 4.32 g of the commercial available 3,3-difluoroazetidine hydrochloride (1:1) (33.3 mmol, 1.0 eq.) and 7.46 mL 1,8-Diazabicyclo[5.4.0]undec-7-ene (50.0 mmol, 1.5 eq.) drop wise. Because of exothermia it was cooled with an ice bath and stirred at rt over night. The reaction mixture was concentrated under reduced pressure and purified by flash chromatography to provide 3.04 g of the target compound.

¹H-NMR (300 MHz, chloroform-d): δ [ppm]=2.47 (t, 2H), 2.88 (t, 2H), 3.72 (t, 4H).

### Intermediate 1-12-1

### Preparation of 2-[1-(4-ethoxy-2,6-difluorobenzyl)-1H-indazol-3-yl]-6-methylpyrimidin-4-ol

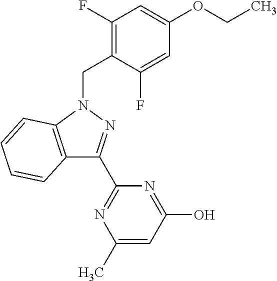 [SMILES: CCOc1cc(F)c(Cn2nc(-c3nc(C)cc(O)n3)c3ccccc32)c(F)c1]

500 mg 1-(4-ethoxy-2,6-difluorobenzyl)-1H-indazole-3-carboximidamide hydrochloride (1:1) 1-1-1 (1.36 mmol, 1.0 eq.) was suspended in 5 mL toluene and treated with 0.20 mL 1,8-Diazabicyclo[5.4.0]undec-7-ene (1.36 mmol, 1.0 eq) and 0.15 mL methyl 3-oxobutanoate (1.36 mmol, 1.0 eq.). It was stirred in a sealed vessel at 110° C. for 6 hours and additional over night. The reaction mixture was diluted with methylene chloride/2-propanole. The organic layer was washed with water and brine and was dried with a water resistant filter, concentrated under reduced pressure and purified by flash chromatography to provide 205 mg of the target compound in 96% purity: 0.52 mmol 38%.

¹H-NMR (400 MHz, DMSO-d₆): δ [ppm]=1.30 (t, 3H), 2.32 (s, 3H), 4.04 (q, 2H), 5.73 (s, 2H), 6.20 (br. s., 1H), 6.65-6.88 (m, 2H), 7.35 (t, 1H), 7.54 (ddd, 1H), 7.83 (d, 1H), 8.40 (d, 1H), 12.02 (br. s., 1H).

The following intermediates were prepared according to the same procedure from the indicated starting materials (SM=starting material):

| | | | |
|---|---|---|---|
| 1-12-2 SM = 1-1-1 | 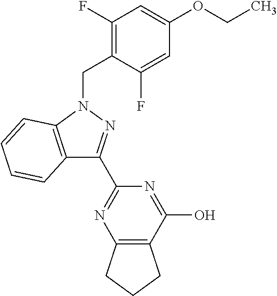 [SMILES: CCOc1cc(F)c(Cn2nc(-c3nc(O)c4c(n3)CCC4)c3ccccc32)c(F)c1] | 2-[1-(4-ethoxy-2,6- difluorobenzyl)-1H- indazol-3-yl]-6,7- dihydro-5H- cyclopenta[d]pyrimidin- 4-ol | ¹H-NMR (400 MHz, DMSO- d₆): δ [ppm] = 1.28 (t, 3H), 1.95-2.10 (m, 2H), 2.61- 2.75 (m, 2H), 2.88 (t, 2H), 3.95-4.11 (m, 2H), 5.70 (s, 2H), 6.68-6.83 (m, 2H), 7.32 (t, 1H), 7.53 (ddd, 1H), 7.82 (d, 1H), 8.33 (d, 1H), 11.44- 11.88 (m, 1H). |
| 1-12-3 SM = 1-1-1 | 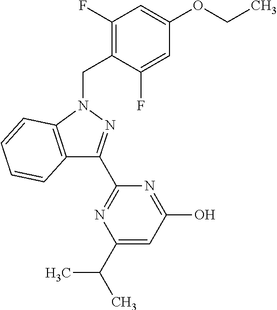 [SMILES: CCOc1cc(F)c(Cn2nc(-c3nc(O)cc(C(C)C)n3)c3ccccc32)c(F)c1] | 2-[1-(4-ethoxy-2,6- difluorobenzyl)-1H- indazol-3-yl]-6- (propan-2- yl)pyrimidin-4-ol | ¹H NMR (300 MHz, DMSO- d₆) δ ppm 1.27 (m, 9 H) 2.82 (dq, 1 H) 4.02 (q, 2 H) 5.71 (s, 2 H) 6.16 (s, 1 H) 6.74 (m, 2 H) 7.35 (m, 1 H) 7.53 (t, 1 H) 7.83 (d, 1 H) 8.38 (d, 1 H) 11.99 (br. s., 1 H) |
| 1-12-4 SM = 1-1-1 | 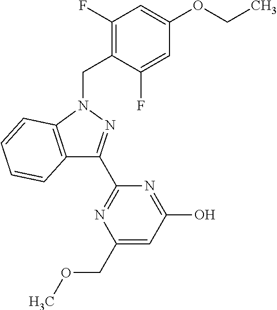 [SMILES: CCOc1cc(F)c(Cn2nc(-c3nc(O)cc(COC)n3)c3ccccc32)c(F)c1] | 2-[1-(4-ethoxy-2,6- difluorobenzyl)-1H- indazol-3-yl]-6- (methoxymethyl) pyrimidin-4-ol | ¹H NMR (300 MHz, DMSO- d₆) δ ppm = 1.28 (t, 3 H) 3.41 (s, 3 H) 4.02 (q, 2 H) 4.39 (s, 2 H) 5.72 (s, 2 H) 6.25 (s, 1 H) 6.75 (d, 2 H) 7.33 (t, 1 H) 7.53 (t, 1 H) 7.83 (d, 1 H) 8.33 (d, 1 H) 12.00 (s, 1 H) |
| 1-12-5 SM = 1-1-1 | 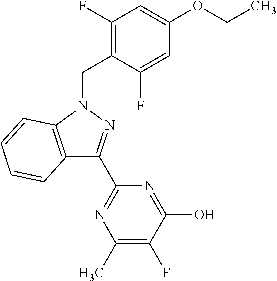 [SMILES: CCOc1cc(F)c(Cn2nc(-c3nc(C)c(F)c(O)n3)c3ccccc32)c(F)c1] | 2-[1-(4-ethoxy-2,6- difluorobenzyl)-1H- indazol-3-yl]-5-fluoro- 6-methylpyrimidin-4-ol | ¹H-NMR (400 MHz, DMSO- d₆) δ [ppm] = 1.30 (t, 3H), 2.35 (s, 3H), 4.04 (q, 2H), 5.68-5.81 (m, 2H), 6.63- 6.89 (m, 2H), 7.35 (t, 1H), 7.54 (td, 1H), 7.83 (d, 1H), 8.36 (d, 1H), 12.84 (br. s., 1H). |
| 1-12-6 SM = 1-1-1 | 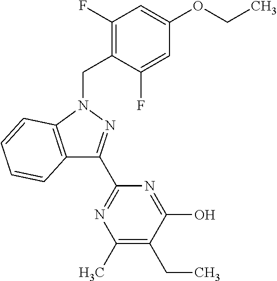 [SMILES: CCOc1cc(F)c(Cn2nc(-c3nc(C)c(CC)c(O)n3)c3ccccc32)c(F)c1] | 2-[1-(4-ethoxy-2,6- difluorobenzyl)-1H- indazol-3-yl]-5-ethyl-6- methylpyrimidin-4-ol | ¹H-NMR (400 MHz, DMSO- d₆): δ [ppm] = 1.03 (t, 3H), 1.28 (t, 3H), 2.35 (s, 3H), 2.45 (q, 2H), 3.95-4.10 (m, 2H), 5.70 (s, 2H), 6.68-6.81 (m, 2H), 7.32 (t, 1H), 7.52 (ddd, 1H), 7.80 (d, 1H), 8.37 (d, 1H), 11.85 (br. s., 1H). |
| 1-12-7 SM = 1-1-1 | 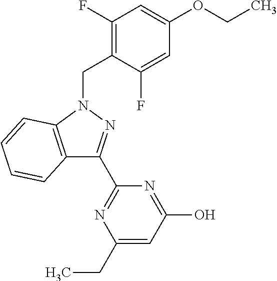 [SMILES: CCOc1cc(F)c(Cn2nc(-c3nc(O)cc(CC)n3)c3ccccc32)c(F)c1] | 2-[1-(4-ethoxy-2,6- difluorobenzyl)-1H- indazol-3-yl]-6- ethylpyrimidin-4-ol | ¹H NMR (500 MHz, DMSO- d₆) δ ppm = 1.26 (m, 6 H) 2.60 (q, 2 H) 4.03 (q, 2 H) 5.71 (s, 2 H) 6.17 (br. s., 1 H) 6.74 (m, 2 H) 7.34 (t, 1 H) 7.53 (ddd, 1 H) 7.82 (d, 1 H) 8.38 (d, 1 H) 11.88 (s, 1 H) |
| 1-12-8 SM = 1-1-1, 1-17-1 | 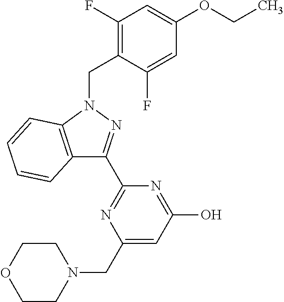 [SMILES: CCOc1cc(F)c(Cn2nc(-c3nc(O)cc(CN4CCOCC4)n3)c3ccccc32)c(F)c1] | 2-[1-(4-ethoxy-2,6- difluorobenzyl)-1H- indazol-3-yl]-6- (morpholin-4- ylmethyl)pyrimidin-4- ol | ¹H NMR (400 MHz, DMSO- d₆) δ ppm = 1.28 (t, 3H), 3.49 (s, 2H), 3.56-3.67 (m, 4H), 3.89-4.14 (m, 2H), 5.71 (s, 2H), 6.34 (s, 1H), 6.65-6.82 (m, 2H), 7.33 (t, 1H), 7.53 (td, 1H), 7.82 (d, 1H), 8.37 (d, 1H), 12.08 (br. s., 1H). 4H not assigned; hidden in DMSO-peak |
| 1-12-9 SM = 1-1-1 | 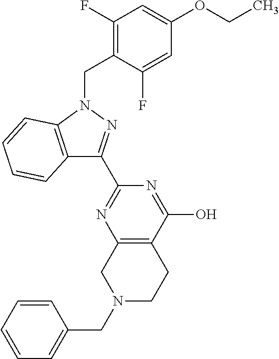 [SMILES: CCOc1cc(F)c(Cn2nc(-c3nc(O)c4c(n3)CN(Cc3ccccc3)CC4)c3ccccc32)c(F)c1] | 7-benzyl-2-[1-(4- ethoxy-2,6- difluorobenzyl)-1H- indazol-3-yl]-5,6,7,8- tetrahydropyrido[3,4- d]pyrimidin-4-ol | ¹H NMR (300 MHz, DMSO- d₆) δ ppm = 1.28 (t, 3H), 2.62- 2.71 (m, 2H), 3.43 (s, 2H), 3.70 (s, 2H), 4.04 (q, 2H), 5.69 (s, 2H), 6.63-6.85 (m, 2H), 7.22-7.43 (m, 6H), 7.51 (t, 1H), 7.80 (d, 1H), 8.25 (d, 1H), 11.98 (br. s., 1H). 2H not assigned, obscured by solvent |
| 1-12-10 SM = 1-1-1 | 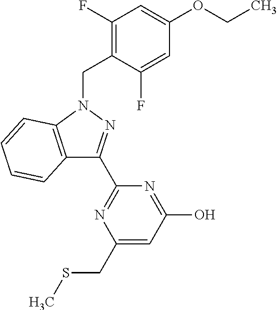 [SMILES: CCOc1cc(F)c(Cn2nc(-c3nc(O)cc(CSC)n3)c3ccccc32)c(F)c1] | 2-[1-(4-ethoxy-2,6- difluorobenzyl)-1H- indazol-3-yl]-6- [(methylsulfanyl) methyl]pyrimidin-4-ol | ¹H NMR (400 MHz, DMSO- d₆) δ ppm 1.28 (t, 3H) 2.10 (s, 3 H) 3.62 (s, 2 H) 4.03 (q, 2 H) 5.72 (s, 2 H) 6.29 (s, 1 H) 6.73 (s, 1 H) 6.76 (s, 1 H) 7.34 (t, 1 H) 7.53 (t, 1 H) 7.83 (d, 1 H) 8.40 (d, 1 H) 12.12 (br. s., 1 H) |
| 1-12-11 SM = 1-1-1 | 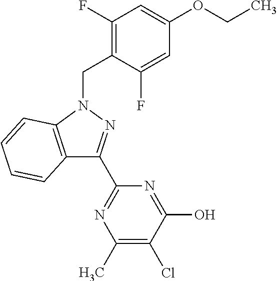 [SMILES: CCOc1cc(F)c(Cn2nc(-c3nc(C)c(Cl)c(O)n3)c3ccccc32)c(F)c1] | 5-chloro-2-[1-(4- ethoxy-2,6- difluorobenzyl)-1H- indazol-3-yl]-6- methylpyrimidin-4-ol | ¹H NMR (400 MHz, DMSO- d₆) δ ppm = 1.30 (t, 3H), 4.04 (q, 2H), 5.74 (s, 2H), 6.68- 6.88 (m, 2H), 7.36 (t, 1H), 7.55 (t, 1H), 7.84 (d, 1H), 8.38 (d, 1H), 12.47-12.99 (m, 1H). |
| 1-12-12 SM = 1-1-1 | 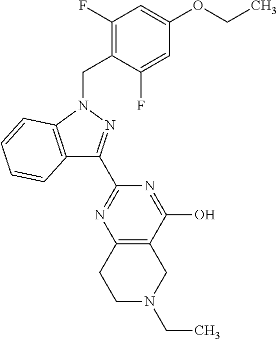 [SMILES: CCOc1cc(F)c(Cn2nc(-c3nc(O)c4c(n3)CCN(CC)C4)c3ccccc32)c(F)c1] | 2-[1-(4-ethoxy-2,6- difluorobenzyl)-1H- indazol-3-yl]-6-ethyl- 5,6,7,8- tetrahydropyrido[4,3- d]pyrimidin-4-ol | ¹H NMR (400 MHz, DMSO- d₆) δ ppm = 1.10 (t, 3H), 1.30 (t, 3H), 2.57 (q, 2H), 2.65- 2.72 (m, 2H), 2.74 (d, 2H), 4.03 (q, 2H), 5.72 (s, 2H), 5.76 (s, 2H), 6.76-6.79 (m, 1H), 6.75 (s, 1H), 7.29-7.38 (m, 1H), 7.54 (ddd, 1H), 7.83 (d, 1H), 8.36 (d, 1H), 11.78- 12.26 (m, 1H). |
| 1-12-13 SM = 1-1-1 | 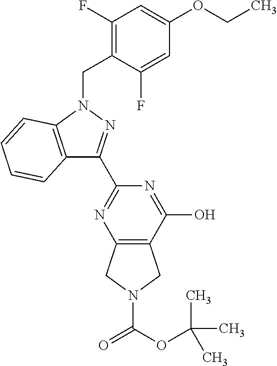 [SMILES: CCOc1cc(F)c(Cn2nc(-c3nc(O)c4c(n3)CN(C(=O)OC(C)(C)C)C4)c3ccccc32)c(F)c1] | tert-butyl 2-[1-(4- ethoxy-2,6- difluorobenzyl)-1H- indazol-3-yl]-4- hydroxy-5,7-dihydro- 6H-pyrrolo[3,4- d]pyrimidine-6- carboxylate | ¹H NMR (400 MHz, DMSO- d₆) δ ppm = 1.30 (t, 3H), 1.47 (s, 9H), 4.05 (q, 2H), 4.40 (d, 2H), 4.53 (d, 2H), 5.74 (s, 2H), 6.66-6.85 (m, 2H), 7.35 (d, 1H), 7.48-7.61 (m, 1H), 7.85 (d, 1H), 8.34 (d, 1H), 12.15-12.53 (br. s, 1H). |
| 1-12-14 SM = 1-1-1 | 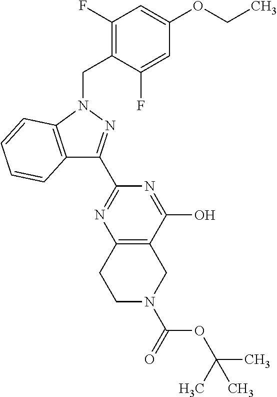 [SMILES: CCOc1cc(F)c(Cn2nc(-c3nc(O)c4c(n3)CCN(C(=O)OC(C)(C)C)C4)c3ccccc32)c(F)c1] | tert-butyl 2-[1-(4- ethoxy-2,6- difluorobenzyl)-1H- indazol-3-yl]-4- hydroxy-7,8- dihydropyrido[4,3- d]pyrimidine-6(5H)- carboxylate | ¹H NMR (400 MHz, DMSO- d₆) δ ppm = 1.30 (t, 3H), 1.39- 1.56 (m, 9H), 2.70-2.81 (m, 2H), 3.63 (t, 2H), 4.04 (q, 2H), 4.15-4.30 (m, 2H), 5.73 (s, 2H), 6.65-6.85 (m, 2H), 7.29- 7.40 (m, 1H), 7.54 (td, 1H), 7.83 (d, 1H), 8.37 (d, 1H). |
| 1-12-15 SM = 1-1-1, 1-17-2 | 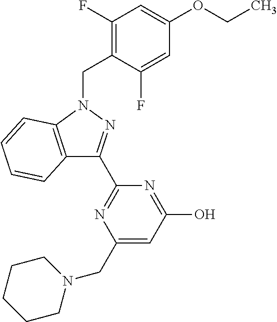 [SMILES: CCOc1cc(F)c(Cn2nc(-c3nc(O)cc(CN4CCCCC4)n3)c3ccccc32)c(F)c1] | 2-[1-(4-ethoxy-2,6- difluorobenzyl)-1H- indazol-3-yl]-6- (piperidin-1- ylmethyl)pyrimidin-4- ol | ¹H NMR (400 MHz, DMSO- d₆) δ ppm = 1.28 (t, 3 H) 1.40 (d, 2 H) 1.52 (m, 4 H) 2.45 (br. s., 4 H) 3.43 (m, 2 H) 4.02 (q, 2 H) 5.73 (s, 2 H) 6.29 (br. s., 1 H) 6.75 (m, 2 H) 7.33 (t, 1 H) 7.52 (m, 1 H) 7.83 (d, 1 H) 8.36 (d, 1 H) 11.94 (m, 1 H). |
| 1-12-16 SM = 1-1-2 | 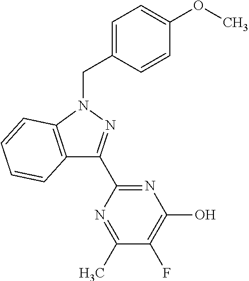 [SMILES: COc1ccc(Cn2nc(-c3nc(C)c(F)c(O)n3)c3ccccc32)cc1] | 5-fluoro-2-[1-(4- methoxybenzyl)-1H- indazol-3-yl]-6- methylpyrimidin-4-ol | ¹H NMR (400 MHz, DMSO- d₆) δ ppm = 2.36 (d, 3H), 3.70 (s, 3H), 5.70 (s, 2H), 6.81- 6.96 (m, 2H), 7.30-7.35 (m, 1H), 7.40 (d, 2H), 7.49 (ddd, 1H), 7.85 (d, 1H), 8.37 (d, 1H), 13.02 (br. s., 1H). |
| 1-12-17 SM = 1-1-1 | 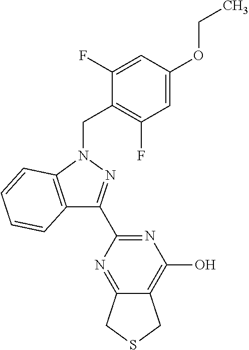 [SMILES: CCOc1cc(F)c(Cn2nc(-c3nc(O)c4c(n3)CSC4)c3ccccc32)c(F)c1] | 2-[1-(4-ethoxy-2,6- difluorobenzyl)-1H- indazol-3-yl]-5,7- dihydrothieno[3,4- d]pyrimidin-4-ol | ¹H NMR (400 MHz, DMSO- d₆) δ ppm = 1.28 (t, 3 H) 3.99- 4.26 (m, 4 H) 4.17-4.34 (m, 2 H) 5.72 (s, 2 H) 6.73 (s, 1 H) 6.77 (s, 1 H) 7.33 (t, 1 H) 7.44-7.60 (m, 1 H) 7.83 (d, 1 H) 8.32 (d, 1 H). |
| 1-12-18 SM = 1-1-2 | 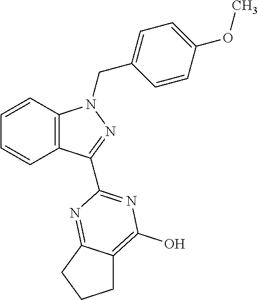 [SMILES: COc1ccc(Cn2nc(-c3nc(O)c4c(n3)CCC4)c3ccccc32)cc1] | 2-[1-(4- methoxybenzyl)-1H- indazol-3-yl]-6,7- dihydro-5H- cyclopenta[d]pyrimdin- 4-ol | ¹H NMR (400 MHz, DMSO- d₆) δ ppm = 1.97-2.13 (m, 2 H) 2.62-2.76 (m, 2 H) 2.80- 2.99 (m, 2 H) 3.69 (s, 3 H) 5.67 (s, 2 H) 6.86 (d, 2 H) 7.24-7.35 (m, 2 H) 7.41 (d, 2 H) 7.83 (d, 1 H) 8.33 (d, 1 H) 9.32 (br. s., 1 H). |
| 1-12-19 SM = 1-1-2 | 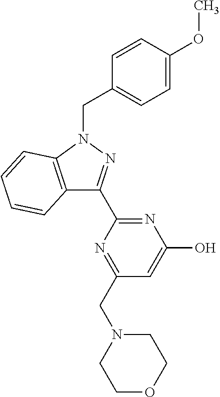 [SMILES: COc1ccc(Cn2nc(-c3nc(O)cc(CN4CCOCC4)n3)c3ccccc32)cc1] | 2-[1-(4- methoxybenzyl)-1H- indazol-3-yl]-6- (morpholin-4- ylmethyl)pyrimidin-4- ol | LC-MS (Methode with NH₃): retention time: 0.80 min MS ES+: 432.3 [M + H]⁺ |
| 1-12-20 SM = 1-1-2 | 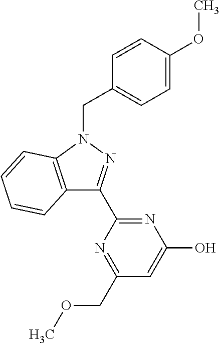 [SMILES: COCc1cc(O)nc(-c2nn(Cc3ccc(OC)cc3)c3ccccc23)n1] | 2-[1-(4- methoxybenzyl)-1H- indazol-3-yl]-6- (methoxymethyl) pyrimidin-4-ol | ¹H NMR (300 MHz, DMSO- d₆) δ ppm = 3.42 (s, 3H), 3.68 (s, 3H), 4.40 (d, 2H), 5.69 (s, 2H), 6.25 (s, 1H), 6.80-6.93 (m, 2H), 7.25-7.35 (m, 1H), 7.40 (d, 2H), 7.43-7.53 (m, 1H), 7.85 (d, 1H), 8.34 (d, 1H), 12.39 (br. s, 1H). |
| 1-12-21 SM = 1-1-1 | 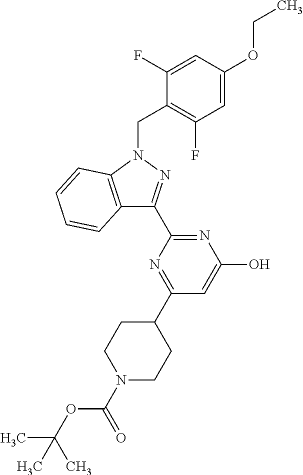 [SMILES: CCOc1cc(F)c(Cn2nc(-c3nc(O)cc(C4CCN(C(=O)OC(C)(C)C)CC4)n3)c3ccccc32)c(F)c1] | tert-butyl 4-{2-[1-(4- ethoxy-2,6- difluorobenzyl)-1H- indazol-3-yl]-6- hydroxypyrimidin-4- yl}piperidine-1- carboxylate | LC-MS (Methode with formic acid): retention time: 1.55 min MS ES+: 466.4 [M + H]⁺ |
| 1-12-22 SM = 1-1-1 + 1-31-1 | 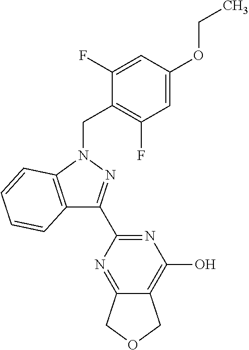 [SMILES: CCOc1cc(F)c(Cn2nc(-c3nc(O)c4c(n3)COC4)c3ccccc32)c(F)c1] | 2-[1-(4-ethoxy-2,6- difluorobenzyl)-1H- indazol-3-yl]-5,7- dihydrofuro[3,4- d]pyrimidin-4-ol | ¹H NMR (400 MHz, DMSO- d₆) δ ppm = 1.30 (t, 3 H) 4.05 (q, 2 H) 4.95 (s, 4 H) 5.74 (s, 2 H) 6.76 (s, 1 H) 6.78 (s, 1 H) 7.35 (t, 1 H) 7.56 (td, 1 H) 7.86 (d, 1 H) 8.31 (d, 1 H). |
| 1-12-23 SM = 1-1-1 | 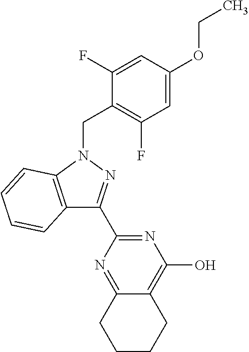 [SMILES: CCOc1cc(F)c(Cn2nc(-c3nc(O)c4c(n3)CCCC4)c3ccccc32)c(F)c1] | 2-[1-(4-ethoxy-2,6- difluorobenzyl)-1H- indazol-3-yl]-5,6,7,8- tetrahydroquinazolin- 4-ol | ¹H NMR (400 MHz, DMSO- d₆) δ ppm = 1.28 (t, 3 H) 1.69- 1.76 (m, 4 H) 2.36-2.44 (m, 2 H) 2.57-2.79 (m, 2 H) 4.03 (q, 2 H) 5.69 (s, 2 H) 6.73 (s, 1 H) 6.75 (s, 1 H) 7.31 (t, 1 H) 7.52 (ddd, 1 H) 7.80 (d, 1 H) 8.34 (d, 1 H) 11.80 (br. s., 1 H). |
| 1-12-24 SM = 1-1-1, 1-32-1 | 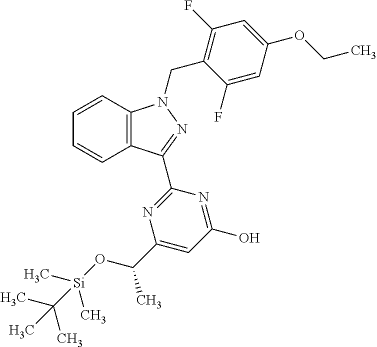 [SMILES: CCOc1cc(F)c(Cn2nc(-c3nc(O)cc([C@H](C)O[Si](C)(C)C(C)(C)C)n3)c3ccccc32)c(F)c1] | 6-[(1S)-1-{[tert- butyl(dimethyl)silyl] oxy}ethyl]-2-[1-(4- ethoxy-2,6- difluorobenzyl)-1H- indazol-3-yl]pyrimidin- 4-ol | ¹H NMR (400 MHz, DMSO- d₆) δ ppm = 0.09 (s, 3H), 0.13 (s, 3H), 0.93 (s, 9H), 1.30 (t, 3H), 1.47 (d, 3H), 4.04 (q, 2H), 4.70-4.84 (m, 1H), 5.75 (s, 2H), 6.33 (s, 1H), 6.71- 6.84 (m, 2H), 7.37 (t, 1H), 7.55 (t, 1H), 7.85 (d, 1H), 8.36 (d, 1H), 12.17 (br. s, 1H). |
| 1-12-25 SM = 1-1-3 | 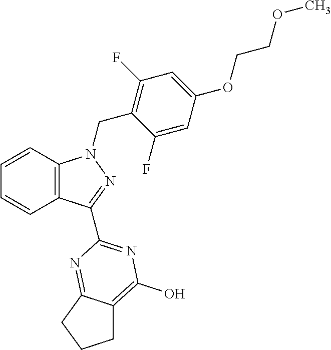 [SMILES: COCCOc1cc(F)c(Cn2nc(-c3nc(O)c4c(n3)CCC4)c3ccccc32)c(F)c1] | 2-{1-[2,6-difluoro-4-(2- methoxyethoxy)benzyl]- 1H-indazol-3-yl}-6,7- dihydro-5H- cyclopenta[d]pyrimidin- 4-ol | ¹H NMR (400 MHz, DMSO- d₆) δ ppm = 1.96-2.06 (m, 2H), 2.64-2.72 (m, 2H), 2.84- 2.92 (m, 2H), 3.26 (s, 3H), 3.58-3.64 (m, 2H), 4.08- 4.14 (m, 2H), 5.70 (s, 2H), 6.73-6.83 (m, 2H), 7.28- 7.35 (m, 1H), 7.49-7.57 (m, 1H), 7.81 (d, 1H), 8.34 (d, 1H), 11.67 (br. s, 1H). |
| 1-12-26 SM = 1-1-1 | 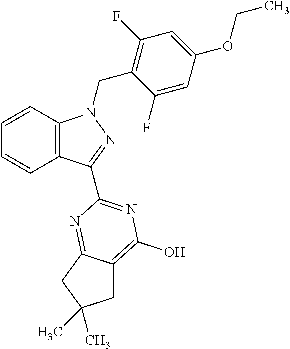 [SMILES: CCOc1cc(F)c(Cn2nc(-c3nc(O)c4c(n3)CC(C)(C)C4)c3ccccc32)c(F)c1] | 2-[1-(4-ethoxy-2,6- difluorobenzyl)-1H- indazol-3-yl]-6,6- dimethyl-6,7-dihydro- 5H- cyclopenta[d]pyrimidin- 4-ol | ¹H NMR (400 MHz, DMSO- d₆) δ ppm = 1.18 (s, 6H), 1.30 (t, 3H), 2.73 (s, 2H), 4.04 (q, 2H), 5.72 (s, 2H), 6.72-6.82 (m, 2H), 7.30-7.38 (m, 1H), 7.51-7.58 (m, 1H), 7.85 (d, 1H), 8.33 (d, 1H), 11.86 (br. s, 1H). —CH2-not detectable |
| 1-12-27 SM = 1-1-3 | 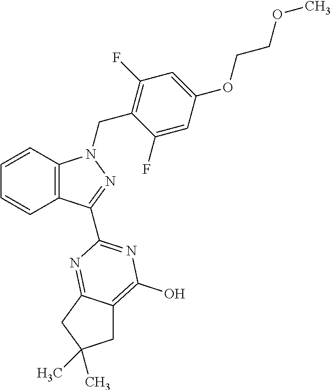 [SMILES: COCCOc1cc(F)c(Cn2nc(-c3nc(O)c4c(n3)CC(C)(C)C4)c3ccccc32)c(F)c1] | 2-{1-[2,6-difluoro-4-(2- methoxyethoxy)benzyl]- 1H-indazol-3-yl}-6,6- dimethyl-6,7-dihydro- 5H- cyclopenta[d]pyrimidin- 4-ol | ¹H NMR (400 MHz, DMSO- d₆) δ ppm = 1.17 (s, 6H), 2.72 (s, 2H), 3.27 (s, 3H), 3.58- 3.67 (m, 2H), 4.09-4.16 (m, 2H), 5.73 (s, 2H), 6.77-6.84 (m, 2H), 7.34 (t, 1H), 7.51- 7.57 (m, 1H), 7.84 (d, 1H), 8.32 (d, 1H), 11.84 (br. s, 1H). —CH2 not detectable |
| 1-12-28 SM = 1-1-3 | 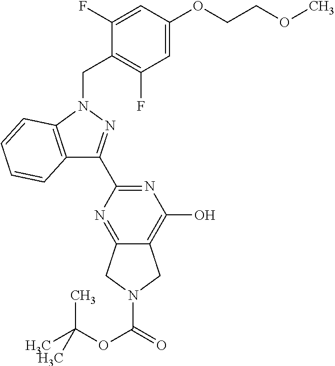 [SMILES: COCCOc1cc(F)c(Cn2nc(-c3nc(O)c4c(n3)CN(C(=O)OC(C)(C)C)C4)c3ccccc32)c(F)c1] | tert-butyl 2-{1-[2,6- difluoro-4-(2- methoxyethoxy)benzyl]- 1H-indazol-3-yl}-4- hydroxy-5,7-dihydro- 6H-pyrrolo[3,4- d]pyrimidine-6- carboxylate | ¹H-NMR (400 MHz, DMSO- d₆): δ [ppm] = 1.46 (s, 9H), 3.27 (s, 3H), 3.62 (m, 2H), 4.11 (m, 2H), 4.39 (br. d., 2H), 4.53 (br. d., 2H), 5.74 (s, 2H), 6.80 (m, 2H), 7.34 (m, 1H), 7.55 (dd, 1H), 7.85 (d, 1H), 8.32 (d, 1H), 12.41 (br. s., 1H). |
| 1-12-29 SM = 1-1-1 | 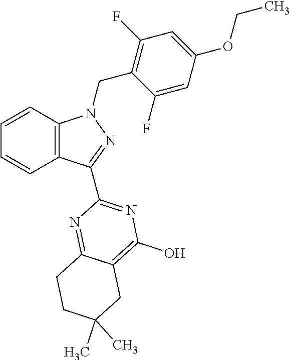 [SMILES: CCOc1cc(F)c(Cn2nc(-c3nc(O)c4c(n3)CCC(C)(C)C4)c3ccccc32)c(F)c1] | 2-[1-(4-ethoxy-2,6- difluorobenzyl)-1H- indazol-3-yl]-6,6- dimethyl-5,6,7,8- tetrahydroquinazolin- 4-ol | ¹H NMR (400 MHz, DMSO- d₆) δ ppm = 0.98 (s, 6H), 1.30 (t, 3H), 1.56 (t, 2H), 2.23 (s, 2H), 2.65-2.74 (m, 2H), 4.04 (q, 2H), 5.72 (s, 2H), 6.71- 6.81 (m, 2H), 7.33 (t, 1H), 7.50-7.58 (m, 1H), 7.83 (d, 1H), 8.36 (d, 1H), 11.91 (br. s, 1H). |
| 1-12-30 SM = 1-1-3 | 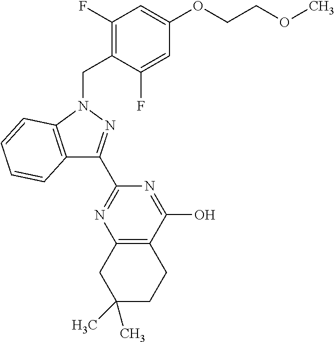 [SMILES: COCCOc1cc(F)c(Cn2nc(-c3nc(O)c4c(n3)CC(C)(C)CC4)c3ccccc32)c(F)c1] | 2-{1-[2,6-difluoro-4-(2- methoxyethoxy)benzyl]- 1H-indazol-3-yl}-7,7- dimethyl-5,6,7,8- tetrahydroquinazolin- 4-ol | ¹H-NMR (400 MHz, DMSO- d₆): δ [ppm] = 0.98 (s, 6H), 1.49 (m, 2H), 2.42 (m, 2H), 2.47 (br. s., 2H), 3.27 (s, 3H), 3.61 (m, 2H), 4.11 (m, 2H), 5.71 (s, 2H), 6.79 (m, 2H), 7.32 (dd, 1H), 7.53 (dd, 1H), 7.81 (d, 1H), 8.36 (d, 1H), 11.92 (br. s., 1H). |
| 1-12-31 SM = 1-1-3 | 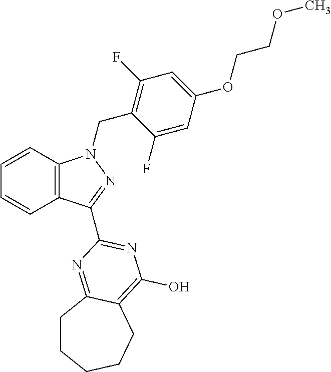 [SMILES: COCCOc1cc(F)c(Cn2nc(-c3nc(O)c4c(n3)CCCCC4)c3ccccc32)c(F)c1] | 2-{1-[2,6-difluoro-4-(2- methoxyethoxy)benzyl]- 1H-indazol-3-yl}- 6,7,8,9-tetrahydro-5H- cyclohepta[d]pyrimidin- 4-ol | ¹H NMR (400 MHz, DMSO- d₆) δ ppm = 1.45-1.56 (m, 2H), 1.60-1.69 (m, 2H), 1.78- 1.87 (m, 2H), 2.65-2.73 (m, 2H), 2.82-2.93 (m, 2H), 3.28 (s, 3H), 3.58-3.66 (m, 2H), 4.08-4.15 (m, 2H), 5.72 (s, 2H), 6.75-6.85 (m, 2H), 7.30- 7.37 (m, 1H), 7.51-7.57 (m, 1H), 7.81 (d, 1H), 8.39 (d, 1H), 11.94 (br. s, 1H). |
| 1-12-32 SM = 1-1-3 | 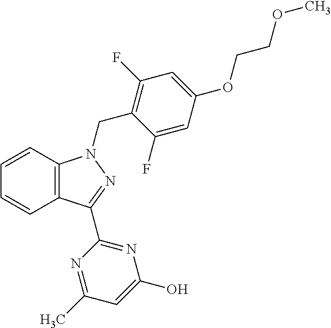 [SMILES: COCCOc1cc(F)c(Cn2nc(-c3nc(C)cc(O)n3)c3ccccc32)c(F)c1] | 2-{1-[2,6-difluoro-4-(2- methoxyethoxy)benzyl]- 1H-indazol-3-yl}-6- methylpyrimidin-4-ol | ¹H NMR (400 MHz, DMSO- d₆) δ ppm = 2.32 (s, 3H), 3.28 (s, 3H), 3.59-3.66 (m, 2H), 4.08-4.15 (m, 2H), 5.73 (s, 2H), 6.20 (br. s., 1H), 6.75- 6.85 (m, 2H), 7.31-7.38 (m, 1H), 7.51-7.58 (m, 1H), 7.84 (d, 1H), 8.39 (d, 1H), 12.00 (br. s, 1H). |
| 1-12-33 SM = 1-1-3 | 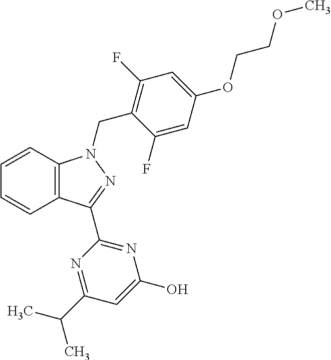 [SMILES: COCCOc1cc(F)c(Cn2nc(-c3nc(O)cc(C(C)C)n3)c3ccccc32)c(F)c1] | 2-{1-[2,6-difluoro-4-(2- methoxyethoxy)benzyl]- 1H-indazol-3-yl}-6- isopropylpyrimidin-4- ol | ¹H NMR (400 MHz, DMSO- d₆) δ ppm = 1.26 (d, 6H), 2.77- 2.91 (m, 1H), 3.28 (s, 3H), 3.58-3.66 (m, 2H), 4.07- 4.16 (m, 2H), 5.74 (s, 2H), 6.17 (s, 1H), 6.75-6.84 (m, 2H), 7.37 (t, 1H), 7.51-7.59 (m, 1H), 7.85 (d, 1H), 8.40 (d, 1H), 12.02 (br. s, 1H). |
| 1-12-34 SM = 1-1-3 | 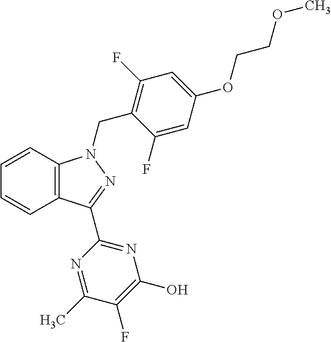 [SMILES: COCCOc1cc(F)c(Cn2nc(-c3nc(C)c(F)c(O)n3)c3ccccc32)c(F)c1] | 2-{1-[2,6-difluoro-4-(2- methoxyethoxy)benzyl]- 1H-indazol-3-yl}-5- fluoro-6- methylpyrimidin-4-ol | ¹H NMR (400 MHz, DMSO- d₆) δ ppm = 2.35 (d, 3H), 3.28 (s, 3H), 3.59-3.67 (m, 2H), 4.08-4.16 (m, 2H), 5.73 (s, 2H), 6.75-6.85 (m, 2H), 7.31- 7.39 (m, 1H), 7.50-7.59 (m, 1H), 7.83 (d, 1H), 8.36 (d, 1H), 12.84 (br. s, 1H). |
| 1-12-35 SM = 1-1-3 | 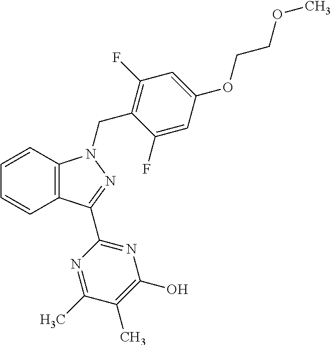 [SMILES: COCCOc1cc(F)c(Cn2nc(-c3nc(C)c(C)c(O)n3)c3ccccc32)c(F)c1] | 2-{1-[2,6-difluoro-4-(2- methoxyethoxy)benzyl]- 1H-indazol-3-yl}-5,6- dimethylpyrimidin-4-ol | ¹H NMR (400 MHz, DMSO- d₆) δ ppm = 1.99 (s, 3H), 2.35 (s, 3H), 3.28 (s, 3H), 3.59- 3.66 (m, 2H), 4.08-4.15 (m, 2H), 5.71 (s, 2H), 6.76-6.84 (m, 2H), 7.33 (t, 1H), 7.50- 7.58 (m, 1H), 7.83 (d, 1H), 8.39 (d, 1H), 11.95 (s, 1H). |
| 1-12-36 SM = 1-1-3 and CAS 27315- 95-3 | 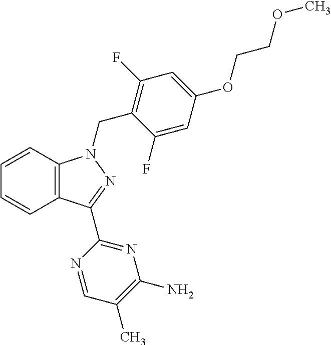 [SMILES: COCCOc1cc(F)c(Cn2nc(-c3ncc(C)c(N)n3)c3ccccc32)c(F)c1] | 2-{1-[2,6-difluoro-4-(2- methoxyethoxy)benzyl]- 1H-indazol-3-yl}-5- methylpyrimidin-4- amine | ¹H NMR (400 MHz, DMSO- d₆) δ ppm = 2.03 (s, 3H), 3.28 (s, 3H), 3.56-3.66 (m, 2H), 4.07-4.18 (m, 2H), 5.66 (s, 2H), 6.70-6.91 (m, 4H), 7.24 (t, 1H), 7.45 (t, 1H), 7.75 (d, 1H), 8.07 (s, 1H), 8.60 (d, 1H). |
| 1-12-37 SM = 1-1-1 | 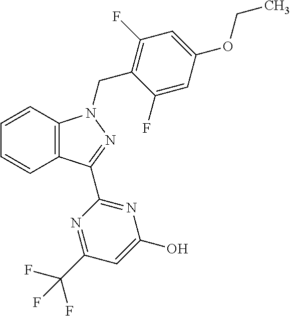 [SMILES: CCOc1cc(F)c(Cn2nc(-c3nc(O)cc(C(F)(F)F)n3)c3ccccc32)c(F)c1] | 2-[1-(4-ethoxy-2,6- difluorobenzyl)-1H- indazol-3-yl]-6- (trifluoromethyl) pyrimidin-4-ol | ¹H NMR (400 MHz, DMSO- d₆) δ ppm = 1.30 (t, 3H), 4.05 (q, 2H), 5.77 (s, 2H), 6.72- 6.91 (m, 3H), 7.35-7.45 (m, 1H), 7.51-7.62 (m, 1H), 7.87 (d, 1H), 8.32 (br. d, 1H), 13.10 (br. s, 1H). |
| 1-12-38 SM = 1-1-3 and 1-4-24 | 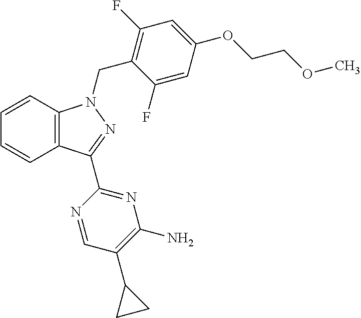 [SMILES: COCCOc1cc(F)c(Cn2nc(-c3ncc(C4CC4)c(N)n3)c3ccccc32)c(F)c1] | 5-cyclopropyl-2-{1- [2,6-difluoro-4-(2- methoxyethoxy)benzyl]- 1H-indazol-3- yl}pyrimidin-4-amine | ¹H NMR (400 MHz, DMSO- d₆) δ ppm = 0.68-0.76 (m, 2H), 0.94-1.04 (m, 2H), 1.67- 1.77 (m, 1H), 3.28 (s, 3H), 3.58-3.66 (m, 2H), 4.08- 4.16 (m, 2H), 5.83 (s, 2H), 6.79-6.86 (m, 2H), 7.43 (t, 1H), 7.59-7.64 (m, 1H), 7.75 (s, 1H), 7.90 (d, 1H), 8.63 (d, 1H), 8.77 (br. s, 1H), 9.28- 9.43 (m, 1H). |
| 1-12-39 SM = 1-1-1 | 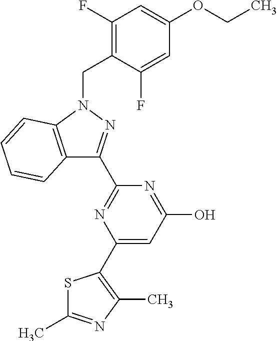 [SMILES: CCOc1cc(F)c(Cn2nc(-c3nc(O)cc(-c4sc(C)nc4C)n3)c3ccccc32)c(F)c1] | 6-(2,4-dimethyl-1,3- thiazol-5-yl)-2-[1-(4- ethoxy-2,6- difluorobenzyl)-1H- indazol-3-yl]pyrimidin- 4-ol | ¹H-NMR (400 MHz, DMSO- d₆): δ [ppm] = 1.29 (t, 3H), 2.61 (s, 3H), 2.67 (s, 3H), 4.04 (q, 2H), 5.75 (s, 2H), 6.51 (s, 1H), 6.77 (m, 2H), 7.41 (dd, 1H), 7.57 (dd, 1H), 7.87 (d, 1H), 8.41 (d, 1H), 12.35 (br. s., 1H). |
| 1-12-40 SM = 1-1-1 | 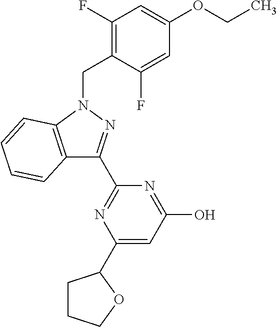 [SMILES: CCOc1cc(F)c(Cn2nc(-c3nc(O)cc(C4CCCO4)n3)c3ccccc32)c(F)c1] | 2-[1-(4-ethoxy-2,6- difluorobenzyl)-1H- indazol-3-yl]-6-[(2rac)- tetrahydrofuran-2- yl]pyrimidin-4-ol | ¹H-NMR (400 MHz, DMSO- d₆): δ [ppm] = 1.29 (t, 3H), 1.93 (m, 3H), 2.37 (m, 1H), 3.87 (m, 1H), 4.00 (m, 1H), 4.03 (q, 2H), 4.79 (m, 1H), 5.73 (s, 2H), 6.26 (s, 1H), 6.76 (m, 2H), 7.35 (dd, 1H), 7.55 (dd, 1H), 7.84 (d, 1H), 8.33 (d, 1H), 12.13 (br. s., 1H). |
| 1-12-41 SM = 1-1-1 | 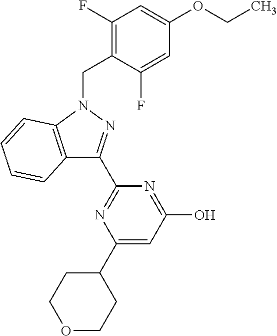 [SMILES: CCOc1cc(F)c(Cn2nc(-c3nc(O)cc(C4CCOCC4)n3)c3ccccc32)c(F)c1] | 2-[1-(4-ethoxy-2,6- difluorobenzyl)-1H- indazol-3-yl]-6- (tetrahydro-2H-pyran- 4-yl)pyrimidin-4-ol | ¹H-NMR (400 MHz, DMSO- d₆): δ [ppm] = 1.29 (t, 3H), 1.77 (m, 2H), 1.81 (m, 2H), 2.78 (m, 1H), 3.45 (ddd, 2H), 3.98 (ddd, 2H), 4.03 (q, 2H), 5.73 (s, 2H), 6.19 (s, 1H), 6.76 (m, 2H), 7.38 (dd, 1H), 7.55 (dd, 1H), 7.85 (d, 1H), 8.37 (d, 1H), 12.09 (br. s., 1H). |
| 1-12-42 SM = 1-1-1 | 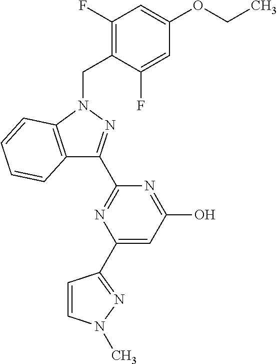 [SMILES: CCOc1cc(F)c(Cn2nc(-c3nc(O)cc(-c4ccn(C)n4)n3)c3ccccc32)c(F)c1] | 2-[1-(4-ethoxy-2,6- difluorobenzyl)-1H- indazol-3-yl]-6-(1- methyl-1H-pyrazol-3- yl)pyrimidin-4-ol | ¹H-NMR (400 MHz, DMSO- d₆): δ [ppm] = 1.29 (t, 3H), 3.96 (s, 3H), 4.04 (q, 2H), 5.75 (s, 2H), 6.70 (s, 1H), 6.77 (m, 2H), 6.97 (d, 1H), 7.41 (dd, 1H), 7.57 (dd, 1H), 7.86 (d, 1H), 7.88 (d, 1H), 8.51 (d, 1H), 12.11 (br. s., 1H). |
| 1-12-43 SM = 1-1-1 | 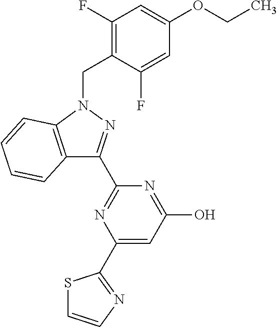 [SMILES: CCOc1cc(F)c(Cn2nc(-c3nc(O)cc(-c4nccs4)n3)c3ccccc32)c(F)c1] | 2-[1-(4-ethoxy-2,6- difluorobenzyl)-1H- indazol-3-yl]-6-(1,3- thiazol-2-yl)pyrimidin- 4-ol | ¹H-NMR (400 MHz, DMSO- d₆): δ [ppm] = 1.29 (t, 3H), 4.04 (q, 2H), 5.77 (s, 2H), 6.77 (m, 2H), 6.92 (s, 1H), 7.44 (dd, 1H), 7.59 (dd, 1H), 7.90 (d, 1H), 8.07 (d, 1H), 8.11 (d, 1H), 8.52 (d, 1H), 12.62 (br. s., 1H). |
| 1-12-44 SM = 1-1-3 | 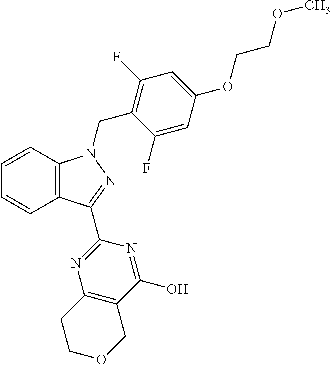 [SMILES: COCCOc1cc(F)c(Cn2nc(-c3nc(O)c4c(n3)CCOC4)c3ccccc32)c(F)c1] | 2-{1-[2,6-difluoro-4-(2- methoxyethoxy)benzyl]- 1H-indazol-3-yl}-7,8- dihydro-5H- pyrano[4,3-d] pyrimidin-4-ol | ¹H NMR (400 MHz, DMSO- d₆) δ ppm = 2.73 (t, 2H), 3.28 (s, 3H), 3.57-3.67 (m, 2H), 3.92 (t, 2H), 4.08-4.17 (m, 2H), 4.44 (s, 2H), 5.73 (s, 2H), 6.73-6.86 (m, 2H), 7.35 (t, 1H), 7.50-7.60 (m, 1H), 7.83 (d, 1H), 8.36 (d, 1H), 12.24 (br. s, 1H). |

### Intermediate 1-13-1

### Preparation of 3-(4-chloro-6,7-dihydro-5H-cyclopenta[d]pyrimidin-2-yl)-1-(4-ethoxy-2,6-difluorobenzyl)-1H-indazole

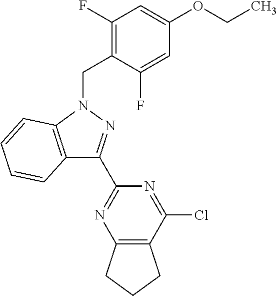 [SMILES: CCOc1cc(F)c(Cn2nc(-c3nc(Cl)c4c(n3)CCC4)c3ccccc32)c(F)c1]

652 mg 2-[1-(4-ethoxy-2,6-difluorobenzyl)-1H-indazol-3-yl]-6,7-dihydro-5H-cyclopenta[d]pyrimidin-4-ol 1-12-2 (1.54 mmol, 1.0 eq.) was suspended in 15 mL toluene and treated with 0.34 mL phosphorus chloride (3.70 mmol, 2.4 eq.) and 0.01 mL DMF (0.15 mmol, 0.1 eq.). It was stirred at 90° C. for 5 hours. The reaction mixture was cooled to room temperature and diluted with ethyl acetate. It was washed once with water and brine, dried with a water resistant filter and concentrated under reduced pressure. The crude product was purified by flash chromatography to provide 408 mg of the target compound, 60%

¹H-NMR (300 MHz, DMSO-d₆): δ [ppm]=1.28 (t, 3H), 2.14 (quin, 2H), 2.99 (t, 2H), 3.12 (t, 2H), 4.03 (q, 2H), 5.73 (s, 2H), 6.76 (d, 2H), 7.32 (t, 1H), 7.51 (t, 1H), 7.82 (d, 1H), 8.48 (d, 1H).

The following intermediates were prepared according to the same procedure from the indicated starting materials (SM=starting material):

| | | | |
|---|---|---|---|
| 1-13-2 SM: = 1- 12-1 | 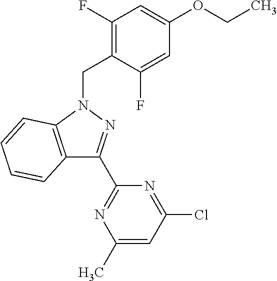 [SMILES: CCOc1cc(F)c(Cn2nc(-c3nc(C)cc(Cl)n3)c3ccccc32)c(F)c1] | 3-(4-chloro-6- methylpyrimidin-2- yl)-1-(4-ethoxy-2,6- difluorobenzyl)-1H- indazole | ¹H-NMR (400 MHz, DMSO- d₆): δ [ppm] = 1.28 (t, 3H), 2.58 (s, 3H), 4.03 (q, 2H), 5.74 (s, 2H), 6.67-6.82 (m, 2H), 7.33 (td, 1H), 7.45-7.56 (m, 2H), 7.82 (d, 1H), 8.51 (d, 1H). |
| 1-13-3 SM = 1- 12-3 | 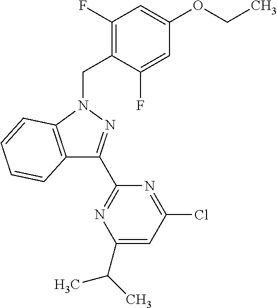 [SMILES: CCOc1cc(F)c(Cn2nc(-c3nc(Cl)cc(C(C)C)n3)c3ccccc32)c(F)c1] | 3-[4-chloro-6- (propan-2- yl)pyrimidin-2-yl]-1- (4-ethoxy-2,6- difluorobenzyl)-1H- indazole | ¹H-NMR (300 MHz, DMSO- d₆): δ [ppm] = 1.28 (t, 3H), 1.33 (d, 6H), 3.12 (dt, 1H), 4.03 (q, 2H), 5.74 (s, 2H), 6.68-6.83 (m, 2H), 7.28- 7.39 (m, 1H), 7.50 (d, 1H), 7.55 (s, 1H), 7.83 (d, 1H), 8.52 (d, 1H). |
| 1-13-4 SM: = 1- 12-4 | 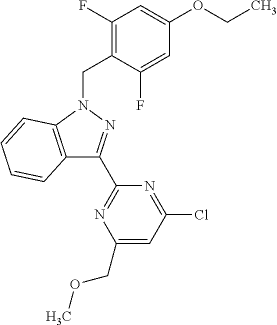 [SMILES: CCOc1cc(F)c(Cn2nc(-c3nc(Cl)cc(COC)n3)c3ccccc32)c(F)c1] | 3-[4-chloro-6- (methoxymethyl)py- rimidin-2-yl]-1-(4- methoxy-2,6- difluorobenzyl)-1H- indazole | ¹H-NMR (500 MHz, DMSO- d₆): δ [ppm] = 1.28 (t, 3H), 3.45 (s, 3H), 4.03 (q, 2H), 4.65 (s, 2H), 5.74 (s, 2H), 6.71-6.80 (m, 2H), 7.34 (t, 1H), 7.48 (s, 1H), 7.52 (ddd, 1H), 7.83 (d, 1H), 8.48 (d, 1H). |
| 1-13-5 SM = 1- 12-5 | 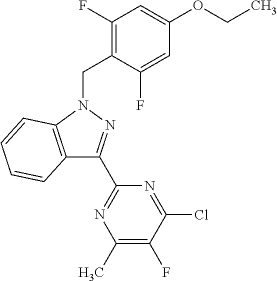 [SMILES: CCOc1cc(F)c(Cn2nc(-c3nc(C)c(F)c(Cl)n3)c3ccccc32)c(F)c1] | 3-(4-chloro-5-fluoro- 6-methylpyrimidin-2- yl)-1-(4-ethoxy-2,6- difluorobenzyl)-1H- indazole | ¹H-NMR (400 MHz, DMSO- d₆): δ [ppm] = 1.28 (t, 3H), 2.61 (d, 3H), 4.03 (q, 2H), 5.73 (s, 2H), 6.66-6.83 (m, 2H), 7.30-7.42 (m, 1H), 7.51 (ddd, 1H), 7.82 (d, 1H), 8.46 (d, 1H). |
| 1-13-6 SM = 1- 12-6 | 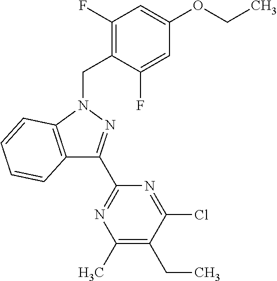 [SMILES: CCOc1cc(F)c(Cn2nc(-c3nc(C)c(CC)c(Cl)n3)c3ccccc32)c(F)c1] | 3-(4-chloro-5-ethyl- 6-methylpyrimidin-2- yl)-1-(4-ethoxy-2,6- difluorobenzyl)-1H- indazole | ¹H-NMR (400 MHz, DMSO- d₆): δ [ppm] = 1.15 (t, 3H), 1.27 (t, 3H), 2.64 (s, 3H), 2.77 (q, 2H), 4.02 (q, 2H), 5.73 (s, 2H), 6.65-6.83 (m, 2H), 7.32 (t, 1H), 7.50 (t, 1H), 7.79 (d, 1H), 8.49 (d, 1H). |
| 1-13-7 SM = 1- 12-7 | 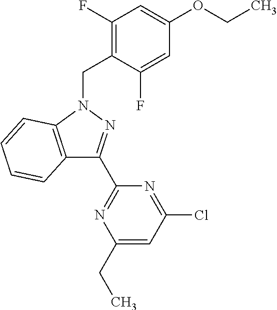 [SMILES: CCOc1cc(F)c(Cn2nc(-c3nc(Cl)cc(CC)n3)c3ccccc32)c(F)c1] | 3-(4-chloro-6- ethylpyrimidin-2-yl)- 1-(4-ethoxy-2,6- difluorobenzyl)-1H- indazole | ¹H-NMR (400 MHz, DMSO- d₆): δ [ppm] = 1.23-1.38 (m, 6H), 2.88 (q, 2H), 4.04 (q, 2H), 5.76 (s, 2H), 6.71-6.84 (m, 2H), 7.36 (t, 1H), 7.48- 7.62 (m, 2H), 7.84 (d, 1H), 8.53 (d, 1H). |
| 1-13-8 SM = 1- 12-8 | 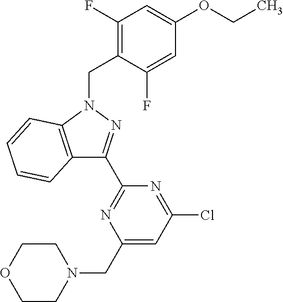 [SMILES: CCOc1cc(F)c(Cn2nc(-c3nc(Cl)cc(CN4CCOCC4)n3)c3ccccc32)c(F)c1] | 3-[4-chloro-6- (morpholin-4- ylmethyl)pyrimidin-2- yl]-1-(4-ethoxy-2,6- difluorobenzyl)-1H- indazole | ¹H-NMR (400 MHz, chloroform-d): δ [ppm] = 1.40 (t, 3 H) 2.63 (m, 4 H) 3.80 (m, 6 H) 3.98 (q, 2 H) 5.78 (s, 2 H) 6.46 (m, 2 H) 7.32 (ddd, 1 H) 7.43 (m, 1 H) 7.54 (m, 2 H) 8.65 (m, 1 H) |
| 1-13-9 SM = 1- 12-9 | 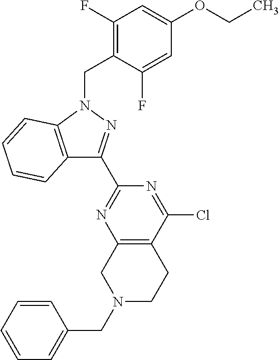 [SMILES: CCOc1cc(F)c(Cn2nc(-c3nc(Cl)c4c(n3)CN(Cc3ccccc3)CC4)c3ccccc32)c(F)c1] | 7-benzyl-4-chloro-2- [1-(4-ethoxy-2,6- difluorobenzyl)-1H- indazol-3-yl]-5,6,7,8- tetrahydropyrido[3,4- d]pyrimidine | ¹H-NMR (400 MHz, chloroform-d): δ [ppm] = 1.28 (t, 3H), 2.81 (s, 4 H), 3.74 (d, 4H), 4.02 (q, 2H), 5.71 (s, 2H), 6.69-6.80 (m, 2H), 7.25- 7.43 (m, 6H), 7.49 (ddd, 1H), 7.80 (d, 1H), 8.43 (d, 1H). |
| 1-13-10 SM = 1- 12-10 | 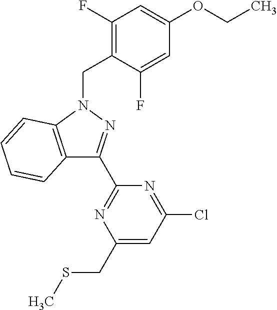 [SMILES: CCOc1cc(F)c(Cn2nc(-c3nc(Cl)cc(CSC)n3)c3ccccc32)c(F)c1] | 3-{4-chloro-6- [(methylsulfanyl)meth- yl]pyrimidin-2-yl}-1- (4-ethoxy-2,6- difluorobenzyl)-1H- indazole | ¹H NMR (400 MHz, DMSO- d₆) δ ppm 1.30 (t, 3 H) 2.13 (s, 3 H) 3.90 (s, 2 H) 4.05 (q, 2 H) 5.76 (s, 2 H) 6.79 (s, 1 H) 6.79 (s, 1 H) 7.24-7.44 (m, 1 H) 7.53 (ddd, 1 H) 7.59- 7.71 (m, 1 H) 7.85 (d, 1 H) 8.51-8.64 (m, 1 H). |
| 1-13-11 SM = 1- 12-11 | 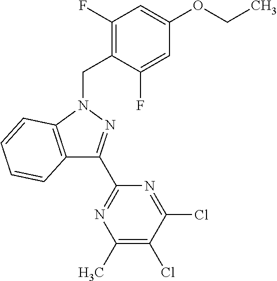 [SMILES: CCOc1cc(F)c(Cn2nc(-c3nc(C)c(Cl)c(Cl)n3)c3ccccc32)c(F)c1] | 3-(4,5-dichloro-6- methylpyrimidin-2- yl)-1-(4-ethoxy-2,6- difluorobenzyl)-1H- indazole | ¹H-NMR (400 MHz, chloroform-d): δ [ppm] = 1.28 (t, 3H), 2.71 (s, 3H), 4.03 (q, 2H), 5.74 (s, 2H), 6.69-6.86 (m, 2H), 7.35 (t, 1H), 7.52 (ddd, 1H), 7.83 (d, 1H), 8.47 (d, 1H). |
| 1-13-12 SM = 1- 12-16 | 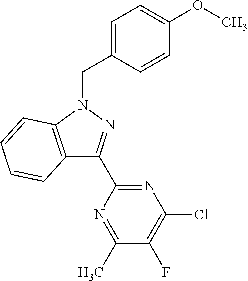 [SMILES: COc1ccc(Cn2nc(-c3nc(C)c(F)c(Cl)n3)c3ccccc32)cc1] | 3-(4-chloro-5-fluoro- 6-methylpyrimidin-2- yl)-1-(4- methoxybenzyl)-1H- indazole | ¹H-NMR (400 MHz, chloroform-d): δ [ppm] = 2.64 (d, 3H), 3.70 (s, 3H), 5.74 (s, 2H), 6.82-6.94 (m, 2H), 7.21- 7.30 (m, 2H), 7.34 (ddd, 1H), 7.48 (ddd, 1H), 7.84 (d, 1H), 8.48 (d, 1H). |
| 1-13-13 SM = 1- 12-18 | 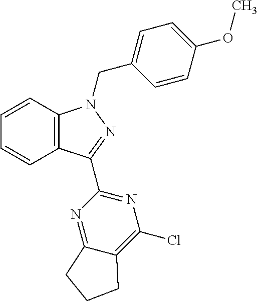 [SMILES: COc1ccc(Cn2nc(-c3nc(Cl)c4c(n3)CCC4)c3ccccc32)cc1] | 3-(4-chloro-6,7- dihydro-5H- cyclopenta[d]pyrimidin- 2-yl)-1-(4- methoxybenzyl)-1H- indazole | ¹H-NMR (400 MHz, DMSO- d₆): δ [ppm] = 2.07-2.24 (m, 2 H) 2.98-3.02 (m, 2 H) 3.11- 3.15 (m, 2 H) 3.70 (s, 3 H) 5.73 (s, 2 H) 6.89 (d, 2 H) 7.22-7.40 (m, 3 H) 7.47 (ddd, 1 H) 7.83 (d, 1 H) 8.50 (d, 1 H). |
| 1-13-14 SM = 1- 12-19 | 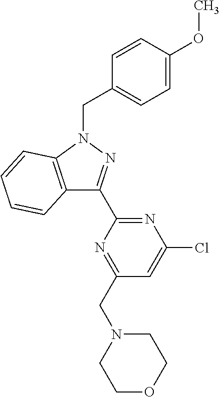 [SMILES: COc1ccc(Cn2nc(-c3nc(Cl)cc(CN4CCOCC4)n3)c3ccccc32)cc1] | 3-[4-chloro-6- (morpholin-4- ylmethyl)pyrimidin-2- yl]-1-(4- methoxybenzyl)-1H- indazole | LC-MS (Methode with formic acid): retention time: 1.10 min MS ES+: 450.2 [M + H]⁺ |

### Intermediate 1-14-1

### Preparation of methyl 4-({2-[1-(4-ethoxy-2,6-difluorobenzyl)-1H-indazol-3-yl]-6-methylpyrimidin-4-yl}amino)nicotinate

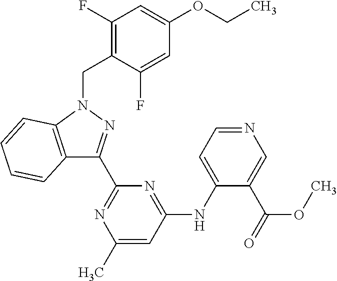 [SMILES: CCOc1cc(F)c(Cn2nc(-c3nc(C)cc(Nc4ccncc4C(=O)OC)n3)c3ccccc32)c(F)c1]

56 mg of 3-(4-chloro-6-methylpyrimidin-2-yl)-1-(4-ethoxy-2,6-difluorobenzyl)-1H-indazole 1-13-2 (0.13 mmol, 1.0 eq.) were dissolved in 1.7 mL of dioxane, and 22 mg methyl 4-aminopyridine-3-carboxylate (0.15 mmol, 1.1 eq.), 3.0 mg palladium(II)acetate (0.01 mmol, 0.1 eq), 11 mg (9,9-dimethyl-9H-xanthene-4,5-diyl)bis(diphenylphosphine) (0.02 mmol, 0.15 eq) and 132 mg cesium carbonate (0.40 mmol, 3.0 eq.) were added. The reaction mixture was stirred at 105° C. under argon atmosphere over night. The reaction mixture was filtered through a HPLC-filter and washed with DCM/2-propanole. The filtrate was concentrated under reduced pressure and crystallized from methanol to receive 100 mg of the target compound, mixed with carboxylic acid as side product. The crude product was used without further purification.

Methyl ester: LC-MS (Method 6): retention time: 1.53 min; MS ES+: 531 [M+H]⁺.

Acid: LC-MS (Method 6): retention time: 0.86 min; MS ES+: 517 [M+H]⁺.

### Intermediate 1-15-1

### Preparation of lithium 4-({2-[1-(4-ethoxy-2,6-difluorobenzyl)-1H-indazol-3-yl]-6-methylpyrimidin-4-yl}amino)nicotinate

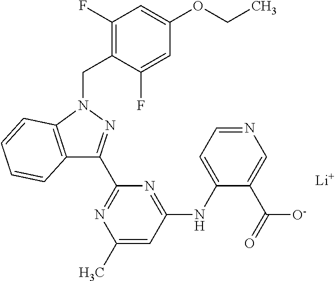 [SMILES: CCOc1cc(F)c(Cn2nc(-c3nc(C)cc(Nc4ccncc4C(=O)[O-])n3)c3ccccc32)c(F)c1 ; [Li+]]

100 mg of methyl 4-({2-[1-(4-ethoxy-2,6-difluorobenzyl)-1H-indazol-3-yl]-6-methylpyrimidin-4-yl}amino)pyridine-3-carboxylate 1-14-1 (0.19 mmol, 1.0 eq.) were dissolved in 0.5 mL methanol and 2 ml THF and treated with 0.25 mL of a lithium hydroxide solution c=1 mol/L (0.25 mmol, 1.3 eq.). It was stirred for 12 hours at rt. The reaction mixture was concentrated under reduced pressure to provide 120 mg of the target compound. The crude product was used without further purification. LC-MS (Method 6): retention time: 0.86 min; MS ES+: 517 [M+H]⁺

### Intermediate 1-16-1

### Preparation of 5-{[tert-butyl(dimethyl)silyl]oxy}pentanenitrile

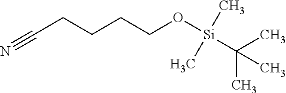

5.0 g of the commercial available tert-butyl(4-chlorobutoxy)dimethylsilane (22.4 mmol, 1.0 eq.) were dissolved in 45 mL DMF at room temperature and treated with 1.65 g sodium cyanide (33.7 mmol, 1.5 eq.) and 3.36 g sodium iodide (22.4 mmol, 1.0 eq.) for 15 hour at 40° C. in a closed vessel. 0.6 g sodium cyanide (12.2 mmol, 0.5 eq.) were added and stirred for further 12 h at 45° C. The reaction mixture was poured onto brine and was extracted with hexane three times. The combined organic layers were dried using a waterresistant filter and the filtrate was concentrated under reduced pressure. The crude product was purified by flash chromatography to provide the 90% pure target compound: 2.8 g, 13.1 mmol, 58%.

¹H-NMR (400 MHz, chloroform-d): δ [ppm]=0.03-0.11 (m, 6H), 0.92 (s, 9H), 1.68 (dd, 2H), 1.72-1.83 (m, 2H), 2.41 (t, 2H), 3.68 (t, 2H).

### Intermediate 1-17-1

### Preparation of methyl 4-(morpholin-4-yl)-3-oxobutanoate

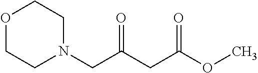

The synthesis of the title compound can be performed as described in US patent publication US2003/0232842.

¹H-NMR (400 MHz, chloroform-d): δ [ppm]=2.49-2.55 (m, 4H), 3.28 (s, 2H), 3.54 (s, 2H), 3.73-3.76 (m, 4H), 3.76 (s, 3H).

The following intermediate was prepared according to the same procedure from commercially available starting materials

| | | | |
|---|---|---|---|
| 1-17-2 | 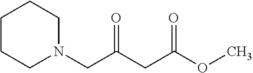 | methyl 3-oxo-4- (piperidin-1- yl)butanoate | LC-MS (Method 5): retention time: 0.80 min MS ES+: 200 [M + H]⁺ |

### Intermediate 1-18-1

### Preparation of 2-[1-(4-ethoxy-2,6-difluorobenzyl)-1H-indazol-3-yl]-6-ethyl-5,6,7,8-tetrahydropyrido[4,3-d]pyrimidin-4-yl trifluoromethanesulfonate

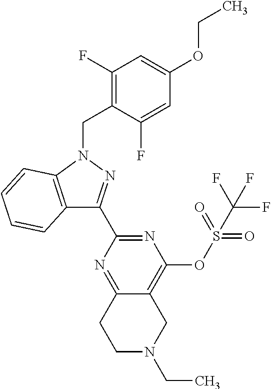 [SMILES: CCOc1cc(F)c(Cn2nc(-c3nc4c(c(OS(=O)(=O)C(F)(F)F)n3)CN(CC)CC4)c3ccccc32)c(F)c1]

To a solution of 250 mg of 2[1-(4-ethoxy-2,6-difluorobenzyl)-1H-indazol-3-yl]-6-ethyl-5,6,7,8-tetrahydropyrido[4,3-d]pyrimidin-4-ol 1-12-12 (0.537 mmol, 1.0 eq.) in 2.5 mL of dichloromethane was added 112 μL triethylamine (0.806 mmol, 1.5 eq.). 644 μL Trifluoromethanesulfonic anhydride (1M in dichloromethane, 0.644 mmol, 1.2 eq) were added dropwise under ice cooling. The reaction mixture was stirred under argon atmosphere for 1 h. Then, a concentrated sodium bicarbonate solution, water and dichloromethane were added. The layers were separated and the aqueous layer was extracted with EE twice. The combined organic layers were dried using a waterresistant filter and the filtrate was dried under reduced pressure. The crude product was purified by flash chromatography to yield 119 mg (37% of theory) of the target compound.

¹H-NMR (400 MHz, chloroform-d): δ [ppm]=2.60-2.74 (m, 2H), 2.88 (br. s., 2H), 3.11 (t, 2H), 3.65 (br. s., 2H), 3.97-4.10 (m, 2H), 5.76 (s, 2H), 6.66-6.87 (m, 2H), 7.23-7.43 (m, 1H), 7.54 (ddd, 1H), 7.87 (d, 1H), 8.46 (d, 1H).

The following intermediates were prepared according to the same procedure from the indicated starting materials (SM=starting material):

| | | | |
|---|---|---|---|
| 1-18-2 SM: 1- 12-13 | 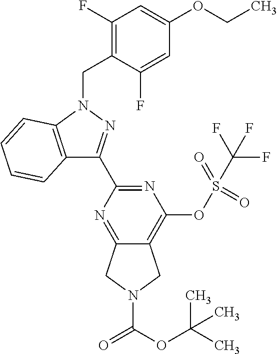 [SMILES: CCOc1cc(F)c(Cn2nc(-c3nc4c(c(OS(=O)(=O)C(F)(F)F)n3)CN(C(=O)OC(C)(C)C)C4)c3ccccc32)c(F)c1] | tert-butyl 2-[1-(4- ethoxy-2,6- difluorobenzyl)-1H- indazol-3-yl]-4- {[(trifluoromethyl) sulfonyl]oxy}-5,7- dihydro-6H- pyrrolo[3,4- d]pyrimidine-6- carboxylate | ¹H-NMR (300 MHz, DMSO- d₆): δ [ppm] = 1.28 (t, 3H), 1.46 (s, 9H), 4.02 (q, 2H), 4.76 (dd, 4H), 5.75 (s, 2H), 6.76 (d, 2H), 7.36 (t, 1H), 7.54 (t, 1H), 7.88 (d, 1H), 8.44 (d, 1H). |
| 1-18-3 SM: 1- 12-14 | 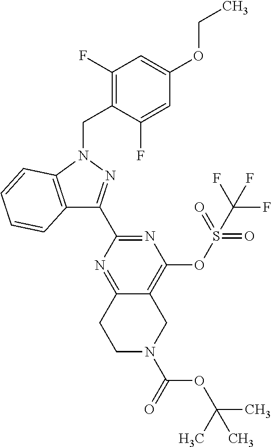 [SMILES: CCOc1cc(F)c(Cn2nc(-c3nc4c(c(OS(=O)(=O)C(F)(F)F)n3)CN(C(=O)OC(C)(C)C)CC4)c3ccccc32)c(F)c1] | tert-butyl 2-[1-(4- ethoxy-2,6- difluorobenzyl)-1H- indazol-3-yl]-4- {[(trifluoromethyl) sulfonyl]oxy}-7,8- dihydropyrido[4,3- d]pyrimidine-6(5H)- carboxylate | ¹H-NMR (400 MHz, DMSO- d₆): δ [ppm] = 1.30 (t, 3H), 1.46 (s, 9H), 3.09 (t, 2H), 3.77 (t, 2H), 4.05 (q, 2H), 4.60 (s, 2H), 5.77 (s, 2H), 6.78 (d, 2H), 7.37 (t, 1H), 7.50-7.61 (m, 1H), 7.87 (d, 1H), 8.47 (d, 1H). |
| 1-18-4 SM: 1- 12-15 | 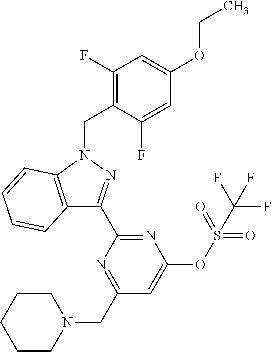 [SMILES: CCOc1cc(F)c(Cn2nc(-c3nc(CN4CCCCC4)cc(OS(=O)(=O)C(F)(F)F)n3)c3ccccc32)c(F)c1] | 2-[1-(4-ethoxy-2,6- difluorobenzyl)-1H- indazol-3-yl]-6- (piperidin-1- ylmethyl)pyrimidin-4- yl trifluoromethane- sulfonate | ¹H-NMR (400 MHz, DMSO- d₆): δ [ppm] = 1.30 (t, 3H), 1.44 (d, 2H), 1.58 (quin, 4H), 3.80 (s, 2H), 4.04 (q, 3H), 5.77 (d, 2H), 6.72-6.86 (m, 2H), 7.37 (t, 1H), 7.51-7.54 (m, 1H), 7.54-7.58 (m ,1H), 7.87 (d, 1H), 8.49 (d, 1H). 4H not assigned. |
| 1-18-5 SM: 1- 12-24 | 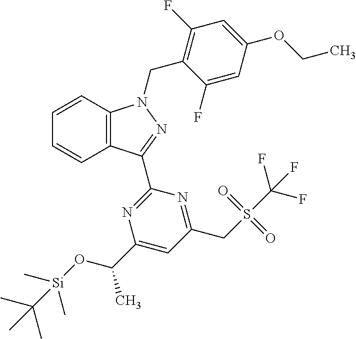 [SMILES: CCOc1cc(F)c(Cn2nc(-c3nc(CS(=O)(=O)C(F)(F)F)cc([C@H](C)O[Si](C)(C)C(C)(C)C)n3)c3ccccc32)c(F)c1] | 6-[(1S)-1-{[tert- butyl(dimethyl)silyl] oxy}ethyl]-2-[1-(4- ethoxy-2,6- difluorobenzyl)-1H- indazol-3- yl]pyrimidin-4-yl trifluoromethane- sulfonate | ¹H-NMR (300 MHz, DMSO- d₆): δ [ppm] = 0.07 (s, 3H), 0.14 (s, 3H), 0.91 (s, 9H), 1.28 (t, 3H), 1.52 (d, 3H), 4.02 (q, 2H), 5.12 (q, 1H), 5.76 (s, 2H), 6.68-6.83 (m, 2H), 7.38 (t, 1H), 7.46 (s, 1H), 7.54 (t, 1H), 7.86 (d, 1H), 8.47 (d, 1H). |
| 1-18-6 SM: 1- 12-30 | 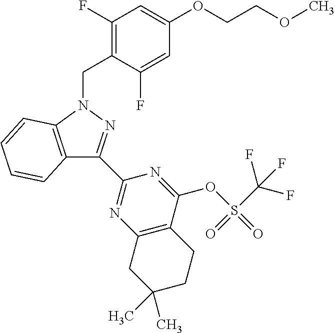 [SMILES: COCCOc1cc(F)c(Cn2nc(-c3nc4c(c(OS(=O)(=O)C(F)(F)F)n3)CCC(C)(C)C4)c3ccccc32)c(F)c1] | 2-{1-[2,6-difluoro-4- (2-methoxy- ethoxy)benzyl]- 1H-indazol-3-yl}- 7,7-dimethyl-5,6,7,8- tetrahydroquinazolin- 4-yl trifluoromethane- sulfonate | ¹H NMR (400 MHz, DMSO- d₆) δ ppm = 1.02 (s, 6H), 1.66 (t, 2H), 2.76 (t, 2H), 2.81 (s, 2H), 3.27 (s, 3H), 3.61 (m, 2H), 4.11 (m, 2H), 5.75 (s, 2H), 6.80 (m, 2H), 7.34 (dd, 1H), 7.53 (dd, 1H), 7.85 (d, 1H), 8.45 (d, 1H). |

### Intermediate 1-19-1

### Preparation of methyl 1-(4-methoxybenzyl)-1H-indazole-3-carboxylate

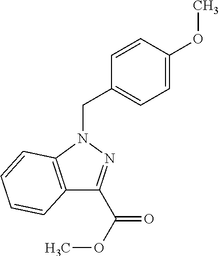 [SMILES: COC(=O)c1nn(Cc2ccc(OC)cc2)c2ccccc12]

20.2 g of methyl 1H-indazole-3-carboxylate (114 mmol, 1.0 eq.) were dissolved in 123 mL of dry DMF and cooled to 0° C. 59.7 g of cesium carbonate (183.1 mmol, 1.6 eq.) were added and stirred for 10 min. 23.3 g of 1-(chloromethyl)-4-methoxybenzene (148 mmol, 1.3 eq.) were added dropwise at 0° C. The mixture was stirred at room temperature for 1 hours under nitrogen atmosphere. Then the reaction mixture was partitioned between water and ethyl ester. The organic layer was dried over silicon filter and concentrated in vacuo. The residue was purified by flash chromatography to yield 20.9 g (60 mmol, 52%) of 85% pure target compound.

¹H NMR (400 MHz, DMSO-d₆) δ [ppm]=3.66 (s, 3H), 3.89 (s, 3H), 5.67 (s, 2H), 6.79-6.90 (m, 2H), 7.20-7.26 (m, 2H), 7.29-7.33 (m, 1H), 7.43-7.47 (m, 1H), 7.84 (d, 1H), 8.05 (dt, 1H).

### Intermediate 1-20-1

### Preparation of 3-(4-chloro-6,7-dihydro-5H-cyclopenta[d]pyrimidin-2-yl)-1-(4-ethoxy-2,6-difluorobenzyl)-1H-indazole

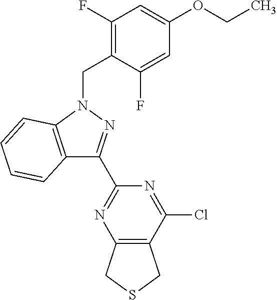 [SMILES: CCOc1cc(F)c(Cn2nc(-c3nc(Cl)c4c(n3)CSC4)c3ccccc32)c(F)c1]

3.29 g (7.47 mmol) of 2-[1-(4-ethoxy-2,6-difluorobenzyl)-1H-indazol-3-yl]-5,7-dihydrothieno[3,4-d]pyrimidin-4-ol 1-12-17 was suspended in 6.36 mL (44.82 mmol) phenylphosphonous dichloride under inert atmosphere. The suspension was heated to 150° C. for 4 h. The reaction mixture cooled to room temperature and extracted with dichloromethane and water. The water layer was extracted two times with dichloromethane. The combined organic layers were dried over sodium sulfate and concentrated under reduced pressure. The crude product was purified by column chromatography (silica gel, 4:1 hexanes/ethyl acetate) to furnish 1.21 g (35% yield) of 4-chloro-2-[1-(4-ethoxy-2,6-difluorobenzyl)-1H-indazol-3-yl]-5,7-dihydrothieno[3,4-d]pyrimidine were isolated.

¹H NMR (400 MHz, DMSO-d₆) δ[ppm]=1.30 (s, 3H) 4.05 (q, 2H) 4.32 (s, 2H) 4.49 (s, 2H) 5.75 (s, 2H) 6.77 (s, 1H) 6.79 (s, 1H) 7.36 (t, 1H) 7.54 (ddd, 1H) 7.86 (d, 1H) 8.49 (d, 1H).

The following intermediates were prepared according to the same procedure from the indicated starting materials (SM=starting material):

| | | | |
|---|---|---|---|
| 1-20-2 SM: 1- 21-1 | 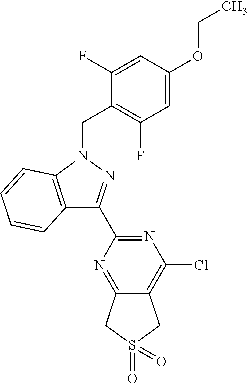 [SMILES: CCOc1cc(F)c(Cn2nc(-c3nc(Cl)c4c(n3)CS(=O)(=O)C4)c3ccccc32)c(F)c1] | 4-chloro-2-[1-(4- ethoxy-2,6- difluorobenzyl)-1H- indazol-3-yl]-5,7- dihydrothieno[3,4- d]pyrimidine 6,6- dioxide | ¹H-NMR (400 MHz, DMSO- d₆): δ [ppm] = 1.28 (t, 3 H) 4.03 (q, 2 H) 4.76 (s, 2 H) 4.95 (s, 2 H) 5.75 (s, 2 H) 6.75 (s, 1 H) 6.78 (s, 1 H) 7.37 (t, 1 H) 7.47-7.63 (m, 1 H) 7.79-7.95 (m, 1 H) 8.47 (d, 1 H). |
| 1-20-3 SM: 1-1- 12-20 | 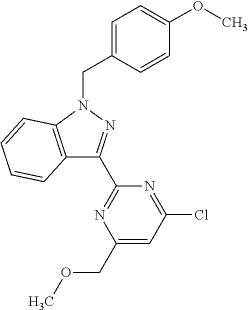 [SMILES: COCc1cc(Cl)nc(-c2nn(Cc3ccc(OC)cc3)c3ccccc23)n1] | 3-[4-chloro-6- (methoxymethyl)py- rimidin-2-yl]-1-(4- methoxybenzyl)-1H- indazole | ¹H-NMR (400 MHz, DMSO- d₆): δ [ppm] = 3.47 (s, 3H), 3.70 (s, 3H), 4.68 (s, 2H), 5.76 (s, 2H), 6.83-6.94 (m, 2H), 7.27 (d, 2H), 7.35 (t, 1H), 7.43-7.55 (m, 2H), 7.85 (d, 1H), 8.51 (d, 1H). |
| 1-20-4 SM: 1- 12-21 | 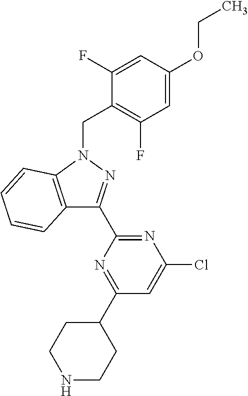 [SMILES: CCOc1cc(F)c(Cn2nc(-c3nc(Cl)cc(C4CCNCC4)n3)c3ccccc32)c(F)c1] | 3-[4-chloro-6- (piperidin-4- yl)pyrimidin-2-yl]-1- (4-methoxy-2,6- difluorobenzyl)-1H- indazole | LC-MS (Methode with formic acid): retention time: 1.21 min MS ES+: 484.2 [M + H]⁺ |
| 1-20-5 SM: 1- 12-22 | 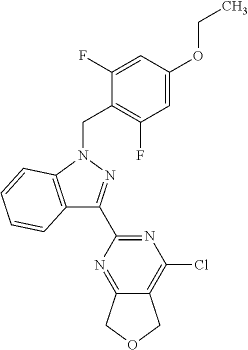 [SMILES: CCOc1cc(F)c(Cn2nc(-c3nc(Cl)c4c(n3)COC4)c3ccccc32)c(F)c1] | 4-chloro-2-[1-(4- ethoxy-2,6- difluorobenzyl)-1H- indazol-3-yl]-5,7- dihydrofuro[3,4- d]pyrimidine | ¹H-NMR (400 MHz, DMSO- d₆): δ [ppm] = 1.30 (t, 3 H) 4.05 (q, 2 H) 5.11-5.28 (m, 4 H) 5.75 (s, 2 H) 6.7 (s, 1 H) 6.80 (s, 1 H) 7.30-7.45 (m, 1 H) 7.55 (ddd, 1 H) 7.87 (d, 1 H) 8.49 (d, 1 H). |
| 1-20-6 SM: 1- 12-23 | 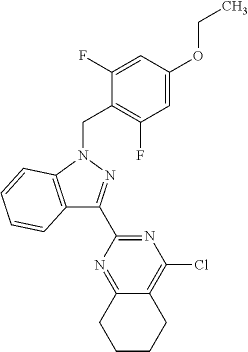 [SMILES: CCOc1cc(F)c(Cn2nc(-c3nc(Cl)c4c(n3)CCCC4)c3ccccc32)c(F)c1] | 4-chloro-2-[1-(4- ethoxy-2,6- difluorobenzyl)-1H- indazol-3-yl]-5,6,7,8- tetrahydroquinazoline | ¹H-NMR (300 MHz, DMSO- d₆): δ [ppm] = 1.28 (t, 3 H) 1.83-1.86 (m, 4 H) 2.71- 2.74 (m, 2 H) 2.90-2.94 (m, 2 H) 4.03 (q, 2 H) 5.72 (s, 2 H) 6.74 (s, 1 H) .677 (s, 1 H) 7.27-7.35 (m, 1 H) 7.50 (ddd, 1 H) 7.80 (d, 1 H) 8.48 (d, 1 H). |
| 1-20-7 SM: 1- 12-25 | 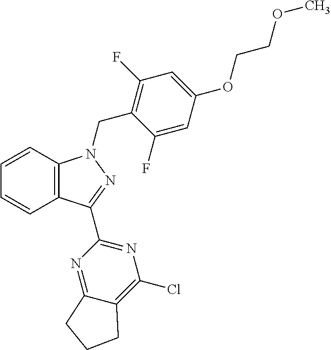 [SMILES: COCCOc1cc(F)c(Cn2nc(-c3nc(Cl)c4c(n3)CCC4)c3ccccc32)c(F)c1] | 3-(4-chloro-6,7- dihydro-5H- cyclopenta[d]pyrimidin- 2-yl)-1-[2,6- difluoro-4-(2- methoxyethoxy) benzyl]-1H-indazole | ¹H-NMR (500 MHz, DMSO- d₆): δ [ppm] = 2.14 (quint, 2H), 2.99 (t, 2H), 3.12 (t, 2H), 3.26 (s, 3H), 3.58-3.64 (m, 2H), 4.07-4.14 (m, 2H), 5.72 (s, 2H), 6.76-6.83 (m, 2H), 7.30- 7.35 (m, 1H), 7.48-7.54 (m, 1H), 7.81 (d, 1H), 8.47 (d, 1H). |
| 1-20-8 SM: 1- 12-26 | 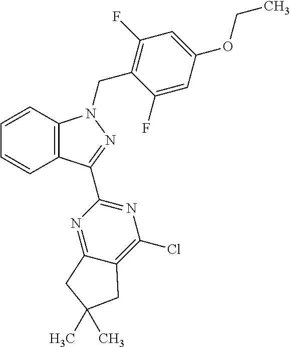 [SMILES: CCOc1cc(F)c(Cn2nc(-c3nc(Cl)c4c(n3)CC(C)(C)C4)c3ccccc32)c(F)c1] | 3-(4-chloro-6,6- dimethyl-6,7- dihydro-5H- cyclopenta[d]pyrimidin- 2-yl)-1-(4-ethoxy- 2,6-difluorobenzyl)- 1H-indazole | ¹H-NMR (400 MHz, DMSO- d₆): δ [ppm] = 1.20 (s, 6H), 1.30 (t, 3H), 2.82 (s, 2H), 2.97 (s, 2H), 4.05 (q, 2H), 5.74 (s, 2H), 6.74-6.83 (m, 2H), 7.31- 7.38 (m, 1H), 7.49-7.56 (m, 1H), 7.84 (d, 1H), 8.49 (d, 1H). |
| 1-20-9 SM: 1- 12-27 | 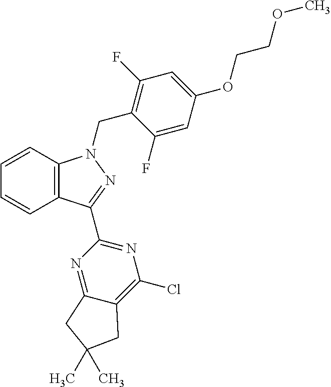 [SMILES: COCCOc1cc(F)c(Cn2nc(-c3nc(Cl)c4c(n3)CC(C)(C)C4)c3ccccc32)c(F)c1] | 3-(4-chloro-6,6- dimethyl-6,7- dihydro-5H- cylcopenta[d]pyrimidin- 2-yl)-1-[2,6- difluoro-4-(2- methoxyethoxy) benzyl]-1H-indazole | ¹H-NMR (400 MHz, DMSO- d₆): δ [ppm] = 1.20 (s, 6H), 2.81 (s, 2H), 2.97 (s, 2H), 3.28 (s, 3H), 3.58-3.67 (m, 2H), 4.09-4.16 (m, 2H), 5.74 (s, 2H), 6.76-6.87 (m, 2H), 7.34 (t, 1H), 7.48-7.56 (m, 1H), 7.84 (d, 1H), 8.49 (d, 1H). |
| 1-20-10 SM: 1- 12-29 | 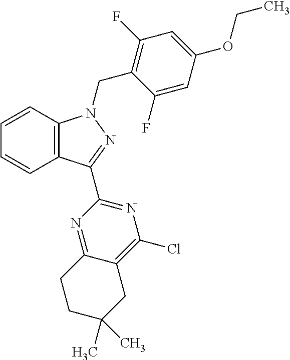 [SMILES: CCOc1cc(F)c(Cn2nc(-c3nc(Cl)c4c(n3)CCC(C)(C)C4)c3ccccc32)c(F)c1] | 4-chloro-2-[1-(4- ethoxy-2,6- difluorobenzyl)-1H- indazol-3-yl]-6,6- dimethyl-5,6,7,8- tetrahydroquinazoline | ¹H-NMR (400 MHz, DMSO- d₆): δ [ppm] = 1.03 (s, 6H), 1.30 (t, 3H), 1.68 (t, 2H), 2.57 (s, 2H), 2.99 (t, 2H), 4.04 (q, 2H), 5.74 (s, 2H), 6.74-6.83 (m, 2H), 7.30-7.37 (m, 1H), 7.48-7.56 (m, 1H), 7.83 (d, 1H), 8.50 (d, 1H). |
| 1-20-11 SM: 1- 12-31 | 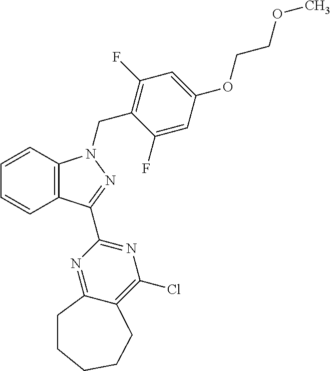 [SMILES: COCCOc1cc(F)c(Cn2nc(-c3nc(Cl)c4c(n3)CCCCC4)c3ccccc32)c(F)c1] | 4-chloro-2-{1-[2,6- difluoro-4-(2- methoxyethoxy)benzyl]- 1H-indazol-3-yl}- 6,7,8,9-tetrahydro- 4H- cyclohepta[d]py- rimidine | ¹H-NMR (400 MHz, DMSO- d₆): δ [ppm] = 1.60-1.75 (m, 4H), 1.83-1.93 (m, 2H), 2.99- 3.06 (m, 2H), 3.10-3.17 (m, 2H), 3.28 (s, 3H), 3.58-3.66 (m, 2H), 4.08-4.16 (m, 2H), 5.74 (s, 2H), 6.77-6.85 (m, 2H), 7.29-7.36 (m, 1H), 7.47- 7.55 (m, 1H), 7.82 (d, 1H), 8.51 (d, 1H). |
| 1-20-12 SM: 1- 12-32 | 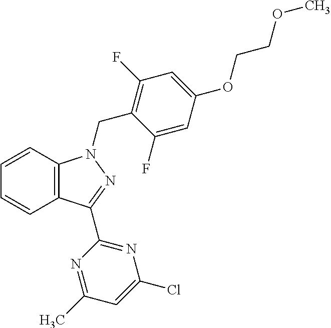 [SMILES: COCCOc1cc(F)c(Cn2nc(-c3nc(C)cc(Cl)n3)c3ccccc32)c(F)c1] | 3-(4-chloro-6- methylpyrimidin-2- yl)-1-[2,6-difluoro-4- (2- methoxyethoxy) benzyl]-1H-indazole | ¹H-NMR (400 MHz, DMSO- d₆): δ [ppm] = 2.59 (s, 3H), 3.28 (s, 3H), 3.60-3.65 (m, 2H), 4.08-4.16 (m, 2H), 5.76 (s, 2H), 6.78-6.86 (m, 2H), 7.32-7.39 (m, 1H), 7.51- 7.57 (m, 2H), 7.84 (d, 1H), 8.52 (d, 1H). |
| 1-20-13 SM: 1- 12-33 | 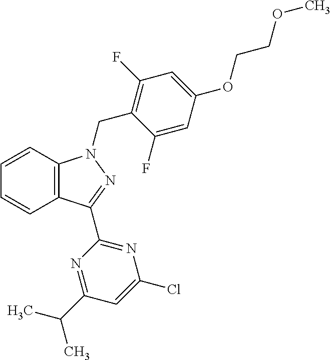 [SMILES: COCCOc1cc(F)c(Cn2nc(-c3nc(Cl)cc(C(C)C)n3)c3ccccc32)c(F)c1] | 3-(4-chloro-6- isopropylpyrimidin-2- yl)-1-[2,6-difluoro-4- (2- methoxyethoxy) benzyl]-1H-indazole | ¹H-NMR (400 MHz, DMSO- d₆): δ [ppm] = 1.35 (d, 6H), 3.06-3.20 (m, 1H), 3.28 (s, 3H), 3.60-3.66 (m, 2H), 4.09- 4.16 (m, 2H), 5.76 (s, 2H), 6.76-6.85 (m, 2H), 7.37 (t, 1H), 7.50-7.56 (m, 1H), 7.56 (s, 1H), 7.84 (d, 1H), 8.54 (d, 1H). |
| 1-20-14 SM: 1- 12-34 | 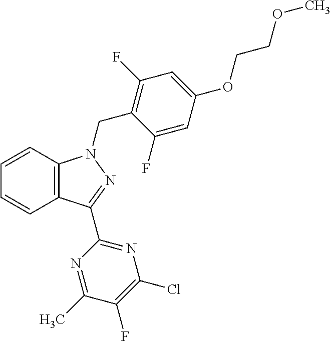 [SMILES: COCCOc1cc(F)c(Cn2nc(-c3nc(C)c(F)c(Cl)n3)c3ccccc32)c(F)c1] | 3-(4-chloro-5-fluoro- 6-methylpyrimidin-2- yl)-1-[2,6-difluoro-4- (2- methoxyethoxy) benzyl]-1H-indazole | ¹H-NMR (400 MHz, DMSO- d₆): δ [ppm] = 2.63 (d, 3H), 3.28 (s, 3H), 3.60-3.66 (m, 2H), 4.10-4.15 (m, 2H), 5.75 (s, 2H), 6.77-6.86 (m, 2H), 7.33-7.39 (m, 1H), 7.50- 7.57 (m, 1H), 7.85 (d, 1H), 8.48 (d, 1H). |
| 1-20-15 SM: 1- 12-35 | 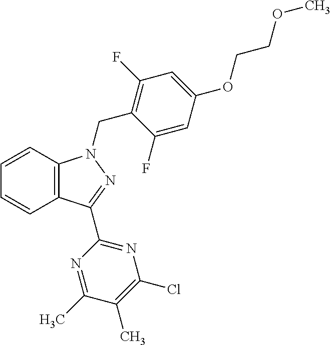 [SMILES: COCCOc1cc(F)c(Cn2nc(-c3nc(C)c(C)c(Cl)n3)c3ccccc32)c(F)c1] | 3-(4-chloro-5,6- dimethylpyrimidin-2- yl)-1-[2,6-difluoro-4- (2- methoxyethoxy) benzyl]-1H-indazole | ¹H-NMR (400 MHz, DMSO- d₆): δ [ppm] = 2.37 (s, 3H), 2.63 (s, 3H), 3.28 (s, 3H), 3.58-3.67 (m, 2H), 4.07- 4.17 (m, 2H), 5.74 (s, 2H), 6.76-6.88 (m, 2H), 7.34 (t, 1H), 7.47-7.56 (m, 1H), 7.83 (d, 1H), 8.52 (d, 1H). |
| 1-20-16 SM: 1- 12-37 | 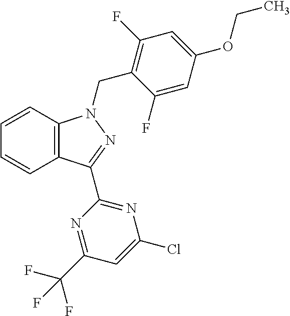 [SMILES: CCOc1cc(F)c(Cn2nc(-c3nc(Cl)cc(C(F)(F)F)n3)c3ccccc32)c(F)c1] | 3-[4-chloro-6- (trifluoromethyl)py- rimidin-2-yl]-1-(4- ethoxy-2,6- difluorobenzyl)-1H- indazole | ¹H-NMR (400 MHz, DMSO- d₆): δ [ppm] = 1.29 (t, 3H), 4.04 (q, 2H), 5.81 (s, 2H), 6.75-6.83 (m, 2H), 7.39- 7.45 (m, 1H), 7.54-7.61 (m, 1H), 7.90 (d, 1H), 8.25 (s, 1H), 8.44 (d, 1H). |
| 1-20-17 SM: 1- 12-44 | 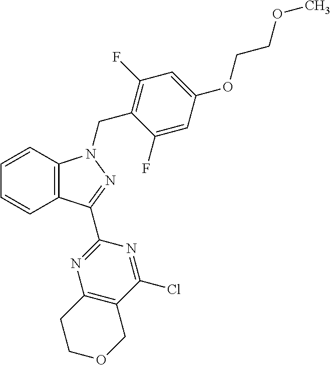 [SMILES: COCCOc1cc(F)c(Cn2nc(-c3nc(Cl)c4c(n3)CCOC4)c3ccccc32)c(F)c1] | 4-chloro-2-{1-[2,6- difluoro-4-[2- methoxyethoxy) benzyl]-1H-indazol-3-yl}- 7,8-dihydro-5H- pyrano[4,3- d]pyrimidine | ¹H-NMR (400 MHz, DMSO- d₆): δ [ppm] = 3.03 (t, 2H), 3.27 (s, 3H), 3.59-3.65 (m, 2H), 4.01-4.16 (m, 4H), 4.73 (s, 2H), 5.76 (s, 2H), 6.77- 6.88 (m, 2H), 7.35 (t, 1H), 7.53 (t, 1H), 7.84 (d, 1H), 8.50 (d, 1H). |

### Intermediate 1-21-1

### Preparation of 2-[1-(4-ethoxy-2,6-difluorobenzyl)-1H-indazol-3-yl]-5, 7-dihydrothieno[3,4-d]pyrimidin-4-ol 6,6-dioxide

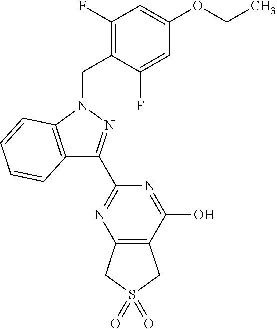 [SMILES: CCOc1cc(F)c(Cn2nc(-c3nc(O)c4c(n3)CS(=O)(=O)C4)c3ccccc32)c(F)c1]

1.2 g (2.72 mmol, 1.0 eq.) of 2-[1-(4-ethoxy-2,6-difluorobenzyl)-1H-indazol-3-yl]-5,7-dihydrothieno[3,4-d]pyrimidin-4-ol 1-12-17 were dissolved in 2 mL chloroform and was cooled to 4° C. 1.69 g 3-chlorobenzenecarboperoxoic acid (65%, 6.0 mmol, 2.2 eq.) dissolved in 1.5 mL chloroform were added slowly. The reaction mixture was stirred at rt for 12 h. After adding chloroform the reaction mixture was washed with sodiumhydrogencarbonate and brine, was dried over silicon filter and concentrated in vacuo. The residue was crystallized from methanol to yield 1.1 g (2.25 mmol, 83%) of 96% pure target compound.

¹H NMR (400 MHz, DMSO-d₆) δ[ppm]=1.30 (t, 3H), 4.05 (q, 2H), 4.33 (s, 2H), 4.63 (s, 2H), 5.75 (s, 2H), 6.68-6.82 (m, 2H), 7.36 (t, 1H), 7.56 (ddd, 1H), 7.86 (d, 1H), 8.26-8.45 (m, 1H), 12.81 (br. s., 1H).

### Intermediate 1-22-1

### Preparation of 2-[(cyclopropylcarbonyl)amino]pyridin-4-aminium chloride

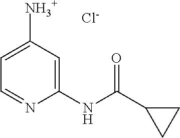 [SMILES: [NH3+]c1ccnc(NC(=O)C2CC2)c1 ; [Cl-]]

450 mg (1.62 mmol) tert-butyl {2-[(cyclopropylcarbonyl)amino]pyridin-4-yl}carbamate 1-23-1 were dissolved in 12.6 mL 4 M hydrogen chloride dissolved in dioxane. The suspension was stirred at room temperature and overnight, concentrated under reduced pressure to one third of the volume and filtered. The isolated crystals were washed with diethyl ether and dried in the air. 420 mg (98% yield, 81% purity, contains NH₄Cl) of 2-[(cyclopropylcarbonyl)amino]pyridin-4-aminium chloride.

¹H NMR (400 MHz, DMSO-d₆) δ [ppm]=0.81-1.06 (m, 4H) 1.95-2.11 (m, 1H) 6.52-6.70 (m, 2H) 7.24-7.37 (m, 1H) 7.92 (br. s., 1H) 8.12 (br. s., 1H) 12.35 (s, 1H) 12.95 (br. s., 1H).

The following intermediate was prepared according to the same procedure from the indicated starting materials (SM=starting material):

| | | | |
|---|---|---|---|
| 1-22-2 SM: 1- 23-2 | 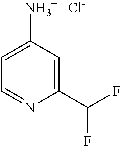 | 2- difluoromethyl) pyridin- 4-amine hydrochloride (1:1) | ¹H-NMR (400 MHz, DMSO-d₆): δ [ppm] = 6.91 (dd, 1 H) 7.00-7.12 (m, 1 H) 7.12-7.42 (m, 1 H) 8.17 (d, 1 H) 8.53 (br. s., 2 H). |

### Intermediate 1-23-1

### Preparation of tert-butyl {2-[(cyclopropylcarbonyl)amino]pyridin-4-yl}carbamate

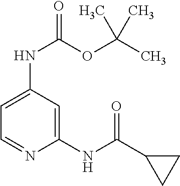 [SMILES: CC(C)(C)OC(=O)Nc1ccnc(NC(=O)C2CC2)c1]

316 mg (2.70 mmol) tert-butyl carbamate, 500 mg (2.07 mmol) N-(4-bromopyridin-2-yl)cyclopropanecarboxamide 1-24-1, 2.03 g (6.22 mmol) cesium carbonate, 47 mg (0.21 mmol) palladium(II) acetate and 180 mg (0.31 mmol) 4,5-bis(diphenylphosphino)-9,9-dimethylxanthene were dissolved under inert atmosphere in 20.00 mL dioxane. The suspension was heated to 90° C. for 2 h, cooled to room temperature and extracted with dichloromethane and water. The organic layer was filtered through a silicon filter and concentrated under reduced pressure. The crude product was purified by column chromatography (silica gel, 3:2 to 0:100 hexanes/ethyl acetate) to furnish 450 mg (78 yield) of tert-butyl {2-[(cyclopropylcarbonyl)amino]pyridin-4-yl}carbamate.

¹H NMR (300 MHz, DMSO-d₆) δ[ppm]=0.61-0.90 (m, 4H) 1.38-1.52 (m, 9H) 1.94-2.02 (m, 1H) 7.08 (dd, 1H) 8.04 (d, 1H) 8.28 (d, 1H) 9.77 (s, 1H) 10.55 (s, 1H).

The following intermediate was prepared according to the same procedure from the indicated starting materials (SM=starting material):

| | | | |
|---|---|---|---|
| 1-23-2 | 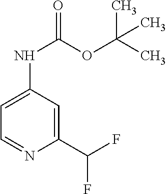 [SMILES: CC(C)(C)OC(=O)Nc1ccnc(C(F)F)c1] | tert-butyl [2- (difluoromethyl)pyri- din-4-yl]carbamate | ¹H-NMR (300 MHz, DMSO- d₆): δ [ppm] = 1.48 (s, 9 H) 6.85 (s, 1 H) 7.49 (dd, 1 H) 7.80 (d, 1 H) 8.43 (d, 1 H) 10.11 (s, 1 H). |

### Intermediate 1-24-1

### Preparation of N-(4-bromopyridin-2-yl)cyclopropanecarboxamide

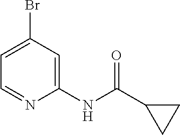 [SMILES: O=C(Nc1cc(Br)ccn1)C1CC1]

1.00 g (5.78 mmol) 2-amino-4-bromopyridine and 0.96 mL (6.89 mmol) triethylamine were dissolved in 5.56 mL dichloromethane and cooled to 0° C. (ice bath). 0.64 mL (6.94 mmol) cyclopropanecarbonyl chloride was added to the reaction mixture and the solution was stirred over night at room temperature. The reaction mixture was evaporated in vacuo and the residue was dissolved in water and ethyl acetate. The organic layer was dried over sodium sulfate and evaporated in vacuo to yield 1.49 g (93% yield) N-(4-bromopyridin-2-yl)cyclopropanecarboxamide.

¹H NMR (400 MHz, DMSO-d₆) δ[ppm]=0.83-1.10 (m, 4H) 1.74-2.13 (m, 1H) 7.33 (dd, 1H) 8.21 (d, 1H) 8.32 (d, 1H) 11.03 (s, 1H).

### Intermediate 1-25-1

### Preparation of 3-(4-chloro-6,7-dihydro-5H-cyclopenta[d]pyrimidin-2-yl)-1-[2,6-difluoro-4-(2-methoxyethoxy)benzyl]-1H-indazole

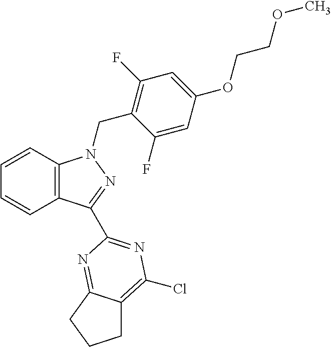 [SMILES: COCCOc1cc(F)c(Cn2nc(-c3nc(Cl)c4c(n3)CCC4)c3ccccc32)c(F)c1]

1.00 g (2.68 mmol) of 3-(4-chloro-6,7-dihydro-5H-cyclopenta[d]pyrimidin-2-yl)-1H-indazole 1-28-1 were dissolved in 7.10 mL DMF. 163 mg (4.06 mmol) of sodium hydride (60% dispersion in mineral oil) were added portionwise under inert atmosphere and stirred 15 minutes at room temperature. The reaction mixture was cooled to 0° C. and 136 mg (0.37 mmol) tetra-n-butylammonium iodide and 1.20 g (4.06 mmol) 2-(bromomethyl)-1,3-difluoro-5-(2-methoxyethoxy)benzene 1-26-1 dissolved in 1 mL DMF were added subsequently. The mixture was stirred overnight, then poured into water and extracted with DCM. The organic layer was dried over sodium sulfate and concentrated under reduced pressure. The crude product was purified by column chromatography (silica gel, 4:1 to 1:1 hexanes/ethyl acetate) and recrystallization in methanol to furnish 0.18 g (8.5 yield) of 3-(4-chloro-6,7-dihydro-5H-cyclopenta[d]pyrimidin-2-yl)-1-[2,6-difluoro-4-(2-methoxyethoxy)benzyl]-1H-indazole.

¹H NMR (400 MHz, DMSO-d₆) δ [ppm]=2.04-2.23 (m, 2H) 2.99 (t, 2H) 3.06-3.19 (m, 2H) 3.26 (s, 3H) 3.52-3.68 (m, 2H) 4.06-4.19 (m, 2H) 5.72 (m, 2H) 6.78 (s, 1H) 6.81 (s, 1H) 7.32 (t, 1H) 7.51 (ddd, 1H) 7.82 (d, 1H) 8.48 (d, 1H).

The following intermediates were prepared according to the same procedure from the indicated starting materials (SM=starting material):

| | | | |
|---|---|---|---|
| 1-25-2 SM = 1- 26-1 + 1-28-2 | 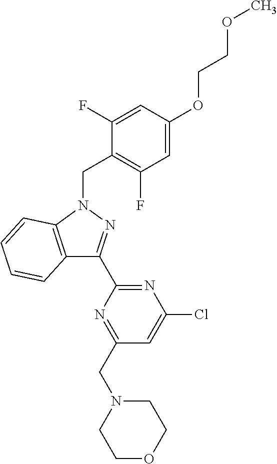 [SMILES: COCCOc1cc(F)c(Cn2nc(-c3nc(Cl)cc(CN4CCOCC4)n3)c3ccccc32)c(F)c1] | 3-[4-chloro-6- (morpholin-4- ylmethyl)pyrimidin-2- yl]-1-[2,6-difluoro-4- (2- methoxyethoxy) benzyl]-1H-indazole | ¹H-NMR (400 MHz, DMSO- d₆): δ [ppm] = 2.48-2.52 (m, 4 H) 3.26 (s, 3 H) 3.52-3.69 (m, 6 H) 3.75 (s, 2 H) 4.11 (dd, 2 H) 5.74 (s, 2 H) 6.78 (s, 1 H) 6.81 (s, 1 H) 7.34 (t, 1 H) 7.43-7.63 (m, 2 H) 7.82 (d, 1 H) 8.51 (d, 1 H). |
| 1-25-3 SM = 1- 28-3 + 1-26-2 DBU instead of NaH | 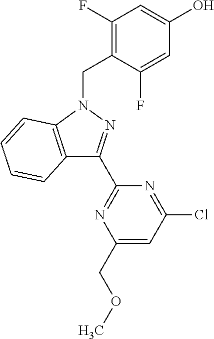 [SMILES: COCc1cc(Cl)nc(-c2nn(Cc3c(F)cc(O)cc3F)c3ccccc23)n1] | 4-({3-[4-chloro-6- (methoxymethyl)pyri- midin-2-yl]-2H- indazol-2-yl}methyl)- 3,5-difluorophenol | ¹H-NMR (400 MHz, DMSO- d₆): δ [ppm] = 3.46 (s, 3H), 4.66 (s, 2H), 5.70 (s, 2H), 6.45-6.54 (m, 2H), 7.30- 7.37 (m, 1H), 7.46-7.56 (m, 2H), 7.81 (d, 1H), 8.48 (d, 1H), 10.51 (br. s, 1H). |

### Intermediate 1-26-1

### Preparation of 2-(bromomethyl)-1,3-difluoro-5-(2-methoxyethoxy)benzene

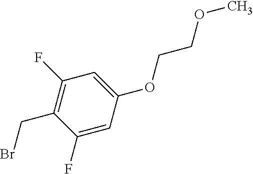

8.40 g (30.80 mmol, ˜80% purity) [2,6-difluoro-4-(2-methoxyethoxy)phenyl]methanol 1-27-1 were dissolved in 9.79 mL 47% hydrogen bromide in water and stirred overnight. The orange solution was poured into 100 mL diethyl ether and the separated organic layer was added dropwise to a saturated sodium bicarbonate solution (gas evolution!). The water layer was extracted twice with diethyl ether, dried over sodium sulfate and concentrated under reduced pressure. 6.30 g (80% yield) 2-(bromomethyl)-1,3-difluoro-5-(2,2-difluoroethoxy)benzene were isolated as an oil and used without further purification in the next step.

¹H NMR (400 MHz, DMSO-d₆) δ[ppm]=3.28 (s, 3H) 3.55-3.68 (m, 2H) 4.09-4.17 (m, 2H) 4.60 (s, 2H) 6.80 (s, 1H) 6.82 (s, 1H).

The following intermediate was prepared according to the same procedure from the indicated starting materials (SM=starting material):

| | | | |
|---|---|---|---|
| 1-26-2 SM = 3,5- difluoro- 4- (hydroxy- methyl) phenol | 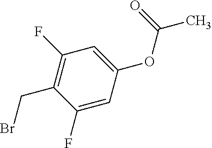 | 4-(bromo methyl)-3,5- difluorophenyl acetate | used without further purifi- cation. |

### Intermediate 1-27-1

### Preparation of [2,6-difluoro-4-(2-methoxyethoxy)phenyl]m ethanol

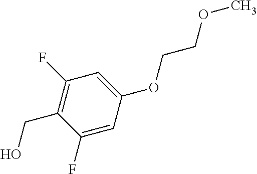

5.00 g (31.23 mmol) 3,5-difluoro-4-(hydroxymethyl)phenol, 5.21 g (37.47 mmol) 2-bromoethyl methyl ether and 21.58 g (156.13 mmol) potassium carbonate were suspended under inert atmosphere in 240 mL DMF. The mixture was stirred at 60° C. overnight, cooled to room temperature, dissolved with ice water and extracted with ethyl acetate. The organic layer was extracted with brine, dried over and concentrated under reduced pressure. 8.40 g (98% yield, ˜80% purity) of [2,6-difluoro-4-(2-methoxyethoxy)phenyl]methanol were isolate and used without further purification in the next step.

¹H NMR (400 MHz, DMSO-d₆) δ[ppm]=3.30 (s, 3H) 3.57-3.70 (m, 2H) 4.06-4.20 (m, 2H) 4.41 (d, 2H) 5.09 (t, 1H) 6.71 (s, 1H) 6.73 (s, 1H).

### Intermediate 1-28-1

### Preparation of [2,6-difluoro-4-(2-methoxyethoxy)phenyl]methanol

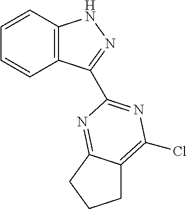 [SMILES: Clc1nc(-c2n[nH]c3ccccc23)nc2c1CCC2]

1.58 g (4.04 mmol) 3-(4-chloro-6,7-dihydro-5H-cyclopenta[d]pyrimidin-2-yl)-1-(4-methoxybenzyl)-1H-indazole 1-13-13 were dissolved in 16 mL 1,2-dichlorethane and 9.34 mL (121.27 mmol) trifluoroacetic acid and 3.58 mL (40.42 mmol) trifluoromethane sulfonic acid were added at room temperature. The solution was heated for 2 h to 75° C., cooled to room temperature and added drop wise to a semi saturated sodium bicarbonate solution. The mixture was stirred 1 h and the precipitated white solid was filtered off. The crude product was purified by titration with methanol, filtration and dried in vacuo at 50° C. 1.50 g (90% yield; 63% purity) of 3-(4-chloro-6,7-dihydro-5H-cyclopenta[d]pyrimidin-2-yl)-1H-indazole were isolated.

¹H NMR (300 MHz, DMSO-d₆) δ[ppm]=2.14 (quin, 2H) 2.99 (t, 2H) 3.12 (t, 2H) 7.23-7.32 (m, 2H) 7.38-7.47 (m, 1H) 7.62 (d, 1H) 8.47 (d, 1H).

The following intermediates were prepared according to the same procedure from the indicated starting materials (SM=starting material):

| | | | |
|---|---|---|---|
| 1-28-2 SM = 1- 13-14 | 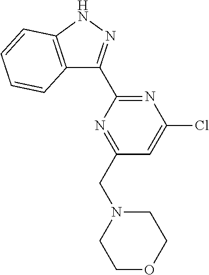 [SMILES: Clc1cc(CN2CCOCC2)nc(-c2n[nH]c3ccccc23)n1] | 3-[4-chloro-6- (morpholin-4- ylmethyl)pyrimidin-2- yl]-1H-indazole | LC-MS (Methode with formic acid): retention time: 1.12 min MS ES+: 330.1 [M + H]⁺ |
| 1-28-3 SM = 1- 20-3 | 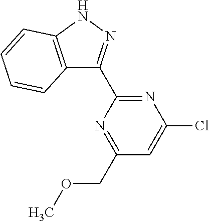 [SMILES: COCc1cc(Cl)nc(-c2n[nH]c3ccccc23)n1] | 3-[4-chloro-6- (methoxymethyl)pyri- midin-2-yl]-1H- indazole | ¹H-NMR (300 MHz, DMSO- d₆): δ [ppm] = 3.46 (s, 3H), 4.66 (s, 2H), 7.22-7.32 (m, 1H), 7.37-7.50 (m, 2H), 7.64 (d, 1H), 8.47 (d, 1H). |

### Intermediate 1-29-1

### Preparation of 1-[4-(2-{[tert-butyl(dimethyl)silyl]oxy}ethoxy)-2,6-difluorobenzyl]-3-[4-chloro-6-(methoxymethyl)pyrimidin-2-yl]-1H-indazole

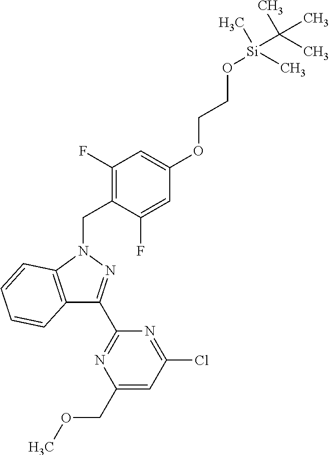 [SMILES: COCc1cc(Cl)nc(-c2nn(Cc3c(F)cc(OCCO[Si](C)(C)C(C)(C)C)cc3F)c3ccccc23)n1]

460 mg of 4-({3-[4-chloro-6-(methoxymethyl)pyrimidin-2-yl]-1H-indazol-1-yl}methyl)-3,5-difluorophenol 1-28-3 (1.1 mmol, 1.0 eq.) was dissolved in 25.5 mL DMF. 763 mg potassium carbonate (5.52 mmol, 5.0 eq.) and 0.36 mL (2-bromoethoxy)(tert-butyl)dimethylsilane (1.66 mmol, 1.5 eq.) were added. The reaction mixture was stirred at 60° C. over night. The reaction mixture was diluted with ethyl acetate and water. The aqueous layer was extracted with ethylacetate twice. The combined organic layers were dried using a waterresistant filter and the filtrate was dried under reduced pressure by rotary evaporation. The crude product was purified by flash chromatography to provide 192 mg (0.29 mmol, 26%) 87% pure target compound.

¹H NMR (400 MHz, DMSO-d₆) δ[ppm]=0.03 (s, 6H), 0.82 (s, 9H), 3.45 (s, 3H), 3.82-3.91 (m, 2H), 3.99-4.10 (m, 2H), 4.65 (s, 2H), 5.74 (s, 2H), 6.71-6.82 (m, 2H), 7.34 (t, 1H), 7.45-7.55 (m, 2H), 7.82 (d, 1H), 8.48 (d, 1H).

### Intermediate 1-30-1

### Preparation of tert-butyl 4-{6-chloro-2-[1-(4-ethoxy-2,6-difluorobenzyl)-1H-indazol-3-yl]pyrimidin-4-yl}piperidine-1-carboxylate

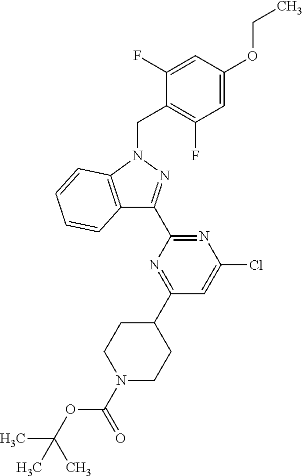 [SMILES: CCOc1cc(F)c(Cn2nc(-c3nc(Cl)cc(C4CCN(C(=O)OC(C)(C)C)CC4)n3)c3ccccc32)c(F)c1]

1.00 g (2.07 mmol) of 3-[4-chloro-6-(piperidin-4-yl)pyrimidin-2-yl]-1-(4-ethoxy-2,6-difluorobenzyl)-1H-indazole 1-20-4 and 0.29 mL (2.07 mmol) of triethylamine were dissolved in 20 mL tetrahydrofuran under inert atmosphere. The solution was cooled to 0° C. and 451 mg (2.07 mmol) di-tert-butyl dicarbonate added at once. The reaction mixture was stirred over night at room temperature and diluted with water and extracted with ethyl acetate. The organic layers were dried over sodium sulfate and concentrated under reduced pressure. The crude product was purified by column chromatography (silica gel, 9:1 to 1:1 hexanes/ethyl acetate) to furnish 300 mg (24% yield) of tert-butyl 4-{6-chloro-2-[1-(4-ethoxy-2,6-difluorobenzyl)-1H-indazol-3-yl]pyrimidin-4-yl}piperidine-1-carboxylate were isolated.

¹H NMR (400 MHz, DMSO-d₆) δ [ppm]=1.29 (t, 3H) 1.44 (s, 9H) 1.65-1.72 (m, 2H) 1.93-1.99 (m, 2H) 2.76-2.93 (m, 2H) 3.06 (m, 1H) 3.96-4.25 (m, 4H) 5.76 (d, 2H) 6.76 (s, 1H) 6.78 (s, 1H) 7.34 (t, 1H) 7.53 (td, 1H) 7.58 (s, 1H) 7.84 (d, 1H) 8.48 (d, 1H).

### Intermediate 1-31-1

### Preparation of methyl 4-oxotetrahydrofuran-3-carboxylate

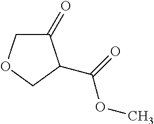

Intermediate 1-31-1 was prepared according to WO 2004/94400 A2; WO 2005/40109 A1; WO 2008/21456 A2; Tetrahedron 1991, 47, 4847 and Tetrahedron Letters 1989, 30, 6129.

### Intermediate 1-32-1

### Preparation of methyl (4S)-4-{[tert-butyl(dimethyl)silyl]oxy}-3-oxopentanoate

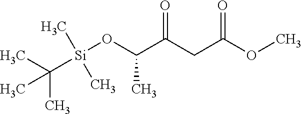 [SMILES: COC(=O)CC(=O)[C@H](C)O[Si](C)(C)C(C)(C)C]

The aperture was evacuated and flushed with argon twice. 2.7 mL methyl acetate (51.6 mmol, 2.4 eq.) were dissolved in 50 mL THF and cooled to −78° C. 43 mL Bis-(trimethylsilyl)-lithiumamide (43 mmol, 2.0 eq.) were added within 20 min. It was stirred 20 minutes at this temperature, then 5.7 mL ethyl (S)-(−)-2-(tert-butyldimethylsilyloxy)propionate (21.5 mmol, 1.0 eq.) dissolved in 75 mL THF was added drop wise within 20 minutes. It was stirred at −78° C. for 1 h, then the reaction mixture was allowed to reach −20° C., later room temperature. The reaction mixture was quenched with saturated ammonium chloride solution. It was extracted three times with ethyl acetate, washed once with water and brine, filtered through a silicone coated filter and dried under reduced pressure. The crude product was used without purification: 90% pure, 5.1 g, 17.7 mmol, 82%.

¹H NMR (400 MHz, DMSO-d₆) δ [ppm]=0.08 (s, 6H), 0.88 (s, 9H), 1.24 (d, 3H), 3.62 (s, 3H), 3.65-3.73 (m, 2H), 4.31 (q, 1H).

### Intermediate 1-33-1

### Preparation of methyl 1-[2,6-difluoro-4-(2-methoxyethoxy)benzyl]-1H-indazole-3-carboxylate

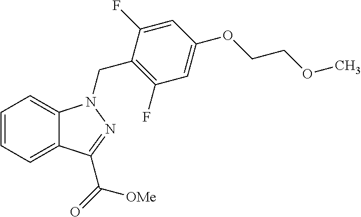 [SMILES: COCCOc1cc(F)c(Cn2nc(C(=O)OC)c3ccccc32)c(F)c1]

5.0 g methyl 1H-indazole-3-carboxylate (28.4 mmol, 1.0 eq.) was dissolved in 45 mL DMF, evaporated and flushed with argon. This procedure was repeated three times. Then 1.25 g sodium hydride (60%, 31.2 mmol, 1.1 eq.) was added portion wise, the reaction mixture was evaporated and flushed with argon three times. It was stirred at room temperature under argon atmosphere for one hour. Then 1.05 g tetra-n-butylammonium iodide (2.8 mmol, 0.1 eq.) was added and the mixture was cooled to 0° C. The reaction mixture was evaporated and flushed with argon three times, then 8.8 g 2-(bromomethyl)-1,3-difluoro-5-(2-methoxyethoxy)benzene 1-34-1 (31.2 mmol, 1.1 eq.) dissolved in 10 mL DMF was added drop wise though a septa and it was stirred at room temperature over night. The reaction mixture was treated with water and methylene chloride. It was extracted three times and washed once with water and brine. The organic layer was filtered through a silicone coated filter and dried under reduced pressure. The crude product was treated with methanol, but no precipitate was formed. The mixture was evaporated and the procedure was repeated with ethyl acetate, hexane and diethyl ether, but no precipitate was formed at all. The crude product was purified by flash chromatography to provide the desired target compound: 3.2 g (97% purity, 29%, 8.3 mmol) and 4.4 g (80% purity, 33%, 9.3 mmol).

¹H-NMR (400 MHz, DMSO-d₆): δ [ppm]=3.27 (s, 3H), 3.58-3.64 (m, 2H), 3.88 (s, 3H), 4.08-4.14 (m, 2H), 5.70 (s, 2H), 6.75-6.83 (m, 2H), 7.31-7.40 (m, 1H), 7.49-7.56 (m, 1H), 7.86 (d, 1H), 8.07 (d, 1H).

### Intermediate 1-34-1

### Preparation of 2-(bromomethyl)-1,3-difluoro-5-(2-methoxyethoxy)benzene

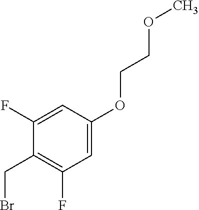

6.0 g [2,6-difluoro-4-(2-methoxyethoxy)phenyl]methanol 1-27-1 (27.4 mmol, 1.0 eq.) was dissolved in 22 mL dichloromethane. 1.9 mL phosphorous tribromide (20.5 mmol, 0.75 eq.) was dissolved in dichloromethane and given to the starting material. The reaction mixture was stirred at room temperature under argon atmosphere for 30 min. The reaction mixture was poured into ice water and the pH was adjusted to 8 by adding saturated sodium hydrogen carbonate solution. It was extracted three times with dichloromethane, washed once with water and brine, filtered through a silicone coated filter and dried under reduced pressure to provide the crude product, which was used without further purification: 8.2 g, 19.2 mmol, 107% yield.

¹H-NMR (300 MHz, DMSO-d₆): δ [ppm]=3.28 (s, 3H), 3.58-3.70 (m, 2H), 4.10-4.18 (m, 2H), 4.60 (s, 2H), 6.73-6.90 (m, 2H)

### Intermediate 1-35-1

### Preparation of tert-butyl 4-chloro-2-{1-[2,6-difluoro-4-(2-methoxyethoxy)benzyl]-1H-indazol-3-yl}-5,7-dihydro-6H-pyrrolo[3,4-d]pyrimidine-6-carboxylate

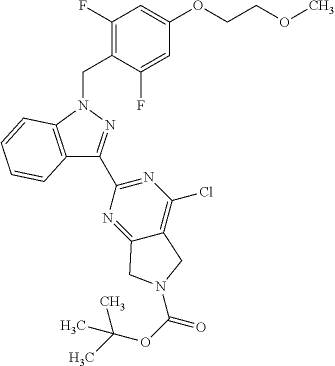 [SMILES: COCCOc1cc(F)c(Cn2nc(-c3nc(Cl)c4c(n3)CN(C(=O)OC(C)(C)C)C4)c3ccccc32)c(F)c1]

2.3 g Tert-butyl 2-{1-[2,6-difluoro-4-(2-methoxyethoxy)benzyl]-1H-indazol-3-yl}-4-hydroxy-5,7-dihydro-6H-pyrrolo[3,4-d]pyrimidine-6-carboxylate 1-12-28 (3.95 mmol, 95% purity) was dissolved in 46 mL N,N-dimethylformamide. 0.58 mL Thionyl chloride (7.89 mmol, 2 eq.) was added to the solution. The reaction mixture was stirred at room temperature for 1 hour. After addition of solid potassium carbonate, the reaction mixture was poured into ice water. The precipitate was filtered off, rinsed with water, taken up with dichloromethane, and evaporated under reduced pressure to provide the crude product, which was used without further purification: 2.21 g, 3.67 mmol, 93% yield, 95% purity.

¹H-NMR (400 MHz, DMSO-d₆): δ [ppm]=1.48 (s, 9H), 3.27 (s, 3H), 3.62 (m, 2H), 4.12 (m, 2H), 4.67 (d, 2H), 4.75 (d, 2H), 5.75 (s, 2H), 6.82 (m, 2H), 7.36 (m, 1H), 7.54 (dd, 1H), 7.86 (d, 1H), 8.48 (d, 1H).

The following intermediates were prepared according to the same procedure from the indicated starting materials (SM=starting material):

| | | | |
|---|---|---|---|
| 1-35-2 SM = 1- 12-39 | 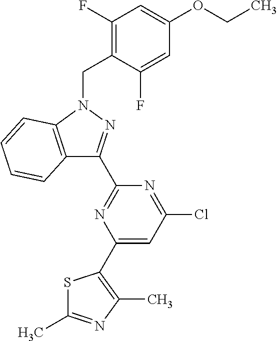 [SMILES: CCOc1cc(F)c(Cn2nc(-c3nc(Cl)cc(-c4sc(C)nc4C)n3)c3ccccc32)c(F)c1] | 3-[4-chloro-6-(2,4- dimethyl-1,3-thiazol- 5-yl)pyrimidin-2-yl]- 1-(4-ethoxy-2,6- difluorobenzyl)-1H- indazole | ¹H-NMR (400 MHz, DMSO- d₆): δ [ppm] = 1.29 (t, 3H), 2.72 (s, 3H), 2.72 (s, 3H), 4.04 (q, 2H), 5.77 (s, 2H), 6.78 (m, 2H), 7.40 (dd, 1H), 7.55 (dd, 1H), 7.72 (s, 1H), 7.87 (d, 1H), 8.52 (d, 1H). |
| 1-35-3 SM = 1- 12-40 | 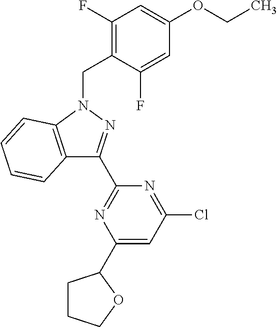 [SMILES: CCOc1cc(F)c(Cn2nc(-c3nc(Cl)cc(C4CCCO4)n3)c3ccccc32)c(F)c1] | 3-{4-chloro-6-[(2rac)- tetrahydrofuran-2- yl]pyrimidin-2-yl}-1- (4-ethoxy-2,6- difluorobenzyl)-1H- indazol | ¹H-NMR (400 MHz, DMSO- d₆): δ [ppm] = 1.29 (t, 3H), 1.86-2.06 (m, 4H), 3.92 (m, 1H), 4.04 (q, 2H), 4.07 (m, 1H), 5.04 (m, 1H), 5.76 (s, 2H), 6.77 (m, 2H), 7.35 (dd, 1H), 7.51 (s, 1H), 7.53 (dd, 1H), 7.84 (d, 1H), 8.48 (d, 1H). |
| 1-35-4 SM = 1- 12-41 | 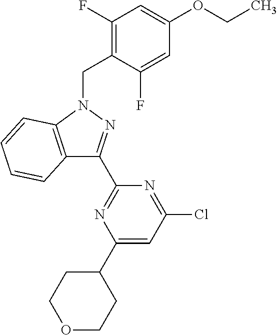 [SMILES: CCOc1cc(F)c(Cn2nc(-c3nc(Cl)cc(C4CCOCC4)n3)c3ccccc32)c(F)c1] | 3-[4-chloro-6- (tetrahydro-2H- pyran-4-yl)pyrimidin- 2-yl]-1-(4-ethoxy- 2,6-difluorobenzyl)- 1H-indazole | ¹H-NMR (400 MHz, DMSO- d₆): δ [ppm] = 1.28 (t, 3H), 1.85 (m, 2H), 1.90 (m, 2H), 3.09 (m, 1H), 3.48 (ddd, 2H), 4.00 (ddd, 2H), 4.03 (q, 2H), 5.75 (s, 2H), 6.77 (m, 2H), 7.37 (dd, 1H), 7.53 (dd, 1H), 7.57 (s, 1H), 7.84 (d, 1H), 8.51 (d, 1H). |
| 1-35-5 SM = 1- 12-42 | 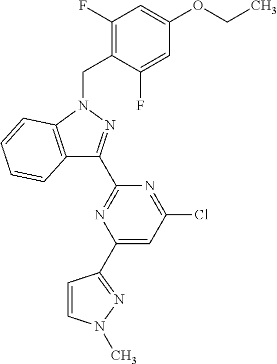 [SMILES: CCOc1cc(F)c(Cn2nc(-c3nc(Cl)cc(-c4ccn(C)n4)n3)c3ccccc32)c(F)c1] | 3-[4-chloro-6-(1- methyl-1H-pyrazol- 3-yl)pyrimidin-2-yl]- 1-(4-ethoxy-2,6- difluorobenzyl)-1H- indazole | ¹H-NMR (400 MHz, DMSO- d₆): δ [ppm] = 1.29 (t, 3H), 4.00 (s, 3H), 4.04 (q, 2H), 5.77 (s, 2H), 6.78 (m, 2H), 7.12 (d, 1H), 7.40 (dd, 1H), 7.55 (dd, 1H), 7.85 (s, 1H), 7.86 (d, 1H), 7.97 (d, 1H), 8.61 (d, 1H). |
| 1-35-6 SM = 1- 12-43 | 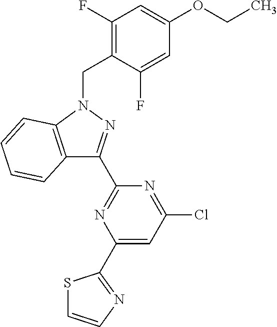 [SMILES: CCOc1cc(F)c(Cn2nc(-c3nc(Cl)cc(-c4nccs4)n3)c3ccccc32)c(F)c1] | 3-[4-chloro-6-(1,3- thiazol-2- yl)pyrimidin-2-yl]-1- (4-ethoxy-2,6- difluorobenzyl)-1H- indazole | ¹H-NMR (400 MHz, DMSO- d₆): δ [ppm] = 1.29 (t, 3H), 4.04 (q, 2H), 5.79 (s, 2H), 6.79 (m, 2H), 7.43 (dd, 1H), 7.57 (dd, 1H), 7.90 (d, 1H), 8.06 (s, 1H), 8.18 (d, 1H), 8.21 (d, 1H), 8.60 (d, 1H). |

### Example Compounds

### Example 2-1-1

### Preparation of 2-[1-(4-ethoxy-2,6-difluorobenzyl)-1H-indazol-3-yl]-5-{2-[(R)-methylsulfinyl]ethyl}-N-(pyridin-4-yl)pyrimidin-4-amine

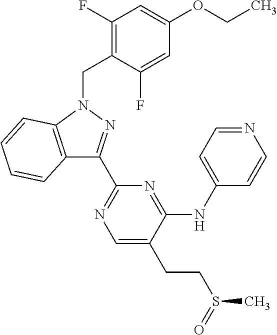 [SMILES: CCOc1cc(F)c(Cn2nc(-c3ncc(CC[S@@](C)=O)c(Nc4ccncc4)n3)c3ccccc32)c(F)c1]

355 mg of 2-[1-(4-ethoxy-2,6-difluorobenzyl)-1H-indazol-3-yl]-5-[2-(methylsulfanyl)ethyl]-N-(pyridin-4-yl)pyrimidin-4-amine 2-2-1 (0.6 mmol, 1.0 eq.) were dissolved in 1.6 mL chloroform and cooled to 0° C. At this temperature 148 mg of 3-chlorobenzenecarboperoxoic acid (77%, 0.66 mmol, 1.1 eq.) were dissolved in 1.5 ml chloroform. The solution was added dropwise. Under cooling the mixture was stirred for 30 min. The reaction mixture was diluted with DCM and quenched with 10%-aqueous sodiumthiosulfat-solution. The layers were separated and the aqueous layer was extracted with DCM twice. The combined organic layers were washed with saturated sodium hydrogencarbonate-solution and dried using a waterresistant filter. The filtrate was concentrated under reduced pressure under vacuo. The crude product was purified by flash chromatography to provide the 100% pure target compound: 310 mg, 0.56 mmol, 94%.

¹H-NMR (300 MHz, DMSO-d₆): δ [ppm]=1.29 (t, 3H), 2.64 (s, 3H), 2.98-3.09 (m, 1H), 3.09-3.25 (m, 3H), 4.04 (q, 2H), 5.71 (s, 2H), 6.74-6.86 (m, 2H), 7.27 (t, 1H), 7.50 (t, 1H), 7.88 (d, 1H), 8.01-8.18 (m, 2H), 8.37-8.49 (m, 3H), 8.51 (s, 1H), 9.20 (s, 1H).

The following compound was prepared according to the same procedure from the indicated starting materials (SM=starting material):

| | | | |
|---|---|---|---|
| 2-1-2 SM = 2-2-2 | 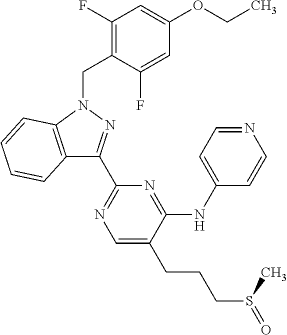 [SMILES: CCOc1cc(F)c(Cn2nc(-c3ncc(CCC[S@@](C)=O)c(Nc4ccncc4)n3)c3ccccc32)c(F)c1] | 2-[1-(4-ethoxy-2,6- difluorobenzyl)-1H- indazol-3-yl]-5-{3- [(R)- methylsulfinyl]propyl}- N-(pyridin-4- yl)pyrimidin-4- amine | ¹H-NMR (300 MHz, DMSO-d₆): δ [ppm] = 1.28 (t, 3H), 1.95-2.09 (m, 2H), 2.57 (s, 3H), 2.67- 2.81 (m, 1H), 2.81-2.96 (m, 3H), 4.03 (q, 2H), 5.70 (s, 2H), 6.74-6.84 (m, 2H), 7.26 (t, 1H), 7.49 (t, 1H), 7.87 (d, 1H), 8.05- 8.21 (m, 2H), 8.37-8.55 (m, 4H), 9.08 (s, 1H). |

### Example 2-2-1

### Preparation of 2-[1-(4-ethoxy-2,6-difluorobenzyl)-1H-indazol-3-yl]-5-[2-(methylsulfanyl)ethyl]-N-(pyridin-4-yl)pyrimidin-4-amine

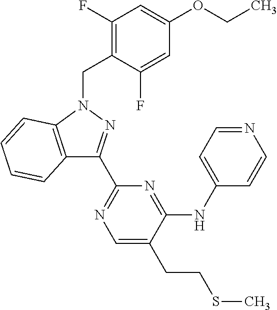 [SMILES: CCOc1cc(F)c(Cn2nc(-c3ncc(CCSC)c(Nc4ccncc4)n3)c3ccccc32)c(F)c1]

930 mg of 2-[1-(4-ethoxy-2,6-difluorobenzyl)-1H-indazol-3-yl]-5-[2-(methylsulfanyl)ethyl]pyrimidin-4-amine 1-3-1 (2.04 mmol, 1.0 eq.) were dissolved in 8 mL of DMF, and 436 mg 4-bromopyridine hydrochloride (1:1) (2.25 mmol, 1.1 eq.), 46 mg palladium(II)acetate (0.20 mmol, 0.1 eq), 177 mg (9,9-dimethyl-9H-xanthene-4,5-diyl)bis(diphenylphosphine) (0.31 mmol, 0.15 eq) and 2.0 g cesium carbonate (6.13 mmol, 3.0 eq.) were added. The reaction mixture was stirred at 105° C. under argon atmosphere over night. The reaction mixture was diluted with water and EE. The layers were separated and the aqueous layer was extracted with EE twice. The combined organic layers were dried using a waterresistant filter and the filtrate was dried under reduced pressure. The crude product was purified by flash chromatography to yield 716 mg (1.18 mmol, 58%) of the 90% pure target compound.

¹H-NMR (300 MHz, DMSO-d₆): δ [ppm]=1.28 (t, 3H), 2.11 (s, 3H), 2.75-2.84 (m, 2H), 2.98-3.09 (m, 2H), 4.03 (q, 2H), 5.69 (s, 2H), 6.68-6.86 (m, 2H), 7.25 (t, 1H), 7.43-7.55 (m, 1H), 7.85 (d, 1H), 8.02-8.12 (m, 2H), 8.37-8.51 (m, 4H), 8.97 (s, 1H).

The following compounds were prepared according to the same procedure from the indicated starting materials (SM=starting material):

| | | | |
|---|---|---|---|
| 2-2-2 SM = 1-3-2 | 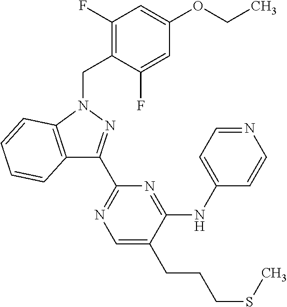 [SMILES: CCOc1cc(F)c(Cn2nc(-c3ncc(CCCSC)c(Nc4ccncc4)n3)c3ccccc32)c(F)c1] | 2-[1-(4-ethoxy- 2,6- difluorobenzyl)- 1H-indazol-3-yl]- 5-[3- (methylsulfanyl) propyl]-N-(pyridin- 4-yl)pyrimidin-4- amine | ¹H-NMR (300MHz, DMSO- d₆): δ [ppm] = 1.28 (t, 3H), 1.87 (quint, 2H), 2.05 (s, 3H), 2.51-2.60 (m, 2H), 2.82 (t, 2H), 4.03 (q, 2H), 5.69 (s, 2H), 6.69-6.84 (m, 2H), 7.24 (t, 1H), 7.48 (t, 1H), 7.85 (d, 1H), 8.02-8.13 (m, 2H), 8.36-8.50 (m, 4H), 8.88 (s, 1H). |
| 2-2-3 SM = 1-3-3 | 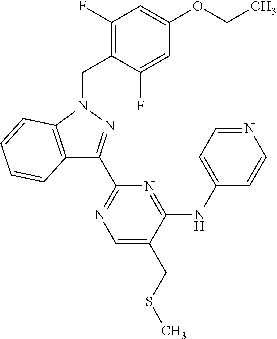 [SMILES: CCOc1cc(F)c(Cn2nc(-c3ncc(CSC)c(Nc4ccncc4)n3)c3ccccc32)c(F)c1] | 2-[1-(4-ethoxy- 2,6- difluorobenzyl)- 1H-indazol-3-yl]- 5- [(methylsulfanyl) methyl]-N- (pyridin-4- yl)pyrimidin-4- amine | ¹H-NMR (300MHz, DMSO- d₆): δ [ppm] = 1.28 (t, 3H), 1.96 (s, 3H), 3.87-4.10 (m, 4H), 5.70 (s, 2H), 6.69-6.88 (m, 2H), 7.19-7.32 (m, 1H), 7.50 (t, 1H), 7.87 (d, 1H), 7.94 (s, 1H), 8.10 (d, 2H), 8.39-8.55 (m, 3H), 8.93 (s, 1H). |
| 2-2-4 SM = 1-6-1 | 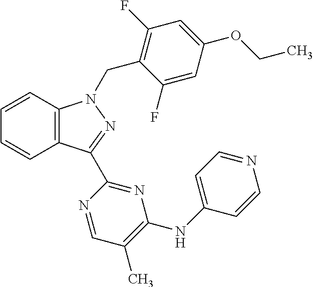 [SMILES: CCOc1cc(F)c(Cn2nc(-c3ncc(C)c(Nc4ccncc4)n3)c3ccccc32)c(F)c1] | 2-[1-(4-ethoxy- 2,6- difluorobenzyl)- 1H-indazol-3-yl]- 5-methyl-N- (pyridin-4- yl)pyrimidin-4- amine | ¹H-NMR (400MHz, DMSO- d₆): δ [ppm] = 1.28 (t, 3H), 2.30 (s, 3H), 4.03 (q, 2H), 5.69 (s, 2H), 6.74-6.81 (m, 2H), 7.20-7.28 (m, 1H), 7.44-7.52 (m, 1H), 7.81- 7.88 (m, 1H), 8.06-8.14 (m, 2H), 8.38-8.48 (m, 4H), 8.86 (s, 1H). |
| 2-2-5 SM = 1-6-1 Side product of 2-2-9 | 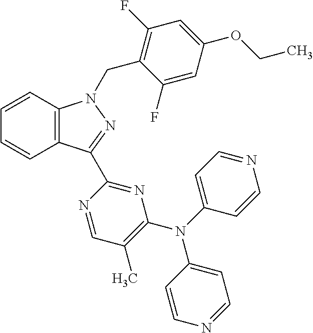 [SMILES: CCOc1cc(F)c(Cn2nc(-c3ncc(C)c(N(c4ccncc4)c4ccncc4)n3)c3ccccc32)c(F)c1] | 2-[1-(4-ethoxy- 2,6- difluorobenzyl)- 1H-indazol-3-yl]- 5-methyl-N,N- di(pyridin-4- yl)pyrimidin-4- amine | ¹H-NMR (400MHz, DMSO- d₆): δ [ppm] = 1.28 (t, 3H), 2.14 (s, 3H), 4.05 (q, 2H), 5.64 (s, 2H), 6.77-6.83 (m, 2H), 7.05-7.14 (m, 1H), 7.32 (d, 2H), 7.38-7.48 (m, 1H), 7.74-7.81 (m, 1H), 7.87-7.93 (m, 1H), 8.04- 8.12 (m, 1H), 8.25 (d, 1H), 8.28 (d, 2H), 8.49 (s, 1H), 8.57-8.63 (m, 1H), 8.65 (s, 1H). |
| 2-2-6 SM = 1-3-4 Dioxane instead of DMF | 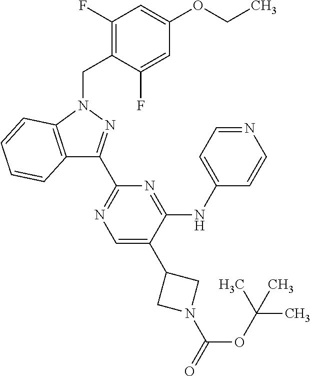 [SMILES: CCOc1cc(F)c(Cn2nc(-c3ncc(C4CN(C(=O)OC(C)(C)C)C4)c(Nc4ccncc4)n3)c3ccccc32)c(F)c1] | tert-butyl 3-{2-[1- (4-ethoxy-2,6- difluorobenzyl)- 1H-indazol-3-yl]- 4-(pyridin-4- ylamino)pyrimidin- 5-yl}azetidine-1- carboxylate | ¹H-NMR (400MHz, DMSO- d₆): δ [ppm] = 1.29 (t, 3H), 1.41 (s, 9H), 3.97-4.12 (m, 4H), 4.12-4.30 (m, 1H), 4.35 (t, 2H), 5.71 (s, 2H), 6.80 (d, 2H), 7.27 (t, 1H), 7.45-7.57 (m, 1H), 7.88 (d, 1H), 8.02 (d, 2H), 8.39-8.54 (m, 3H), 8.71 (d, 2H). |
| 2-2-7 SM = 1-3-5 Dioxane instead of DMF | 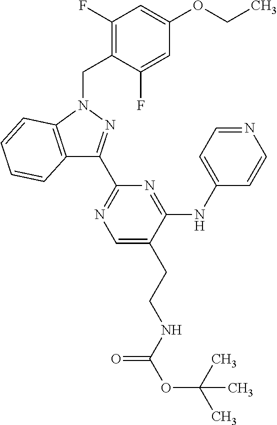 [SMILES: CCOc1cc(F)c(Cn2nc(-c3ncc(CCNC(=O)OC(C)(C)C)c(Nc4ccncc4)n3)c3ccccc32)c(F)c1] | tert-butyl (2-{2-[1- (4-ethoxy-2,6- difluorobenzyl)- 1H-indazol-3-yl]- 4-(pyridin-4- ylamino)pyrimidin- 5- yl}ethyl)carbamate | ¹H-NMR (300MHz, DMSO- d₆): δ [ppm] = 1.17-1.42 (m, 12H), 2.84 (t, 2H), 3.12- 3.26 (m, 2H), 4.03 (q, 2H), 5.69 (s, 2H), 6.74-6.83 (m, 2H), 7.11 (br. s., 1H), 7.18- 7.30 (m, 1H), 7.49 (t, 1H), 7.85 (d, 1H), 8.11 (d, 2H), 8.36 (s, 1H), 8.39-8.50 (m, 3H), 9.06 (s, 1H). |
| 2-2-8 SM = 1-3-6 Dioxane instead of DMF | 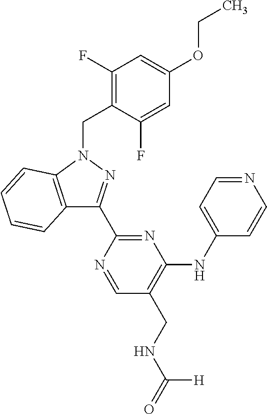 [SMILES: CCOc1cc(F)c(Cn2nc(-c3ncc(CNC=O)c(Nc4ccncc4)n3)c3ccccc32)c(F)c1] | N-({2-[1-(4- ethoxy-2,6- difluorobenzyl)- 1H-indazol-3-yl]- 4-(pyridin-4- ylamino)pyrimidin- 5- yl}methyl) formamide | ¹H-NMR (300MHz, DMSO- d₆): δ [ppm] = 1.28 (t, 3H), 4.03 (q, 2H), 4.43 (d, 2H), 5.71 (s, 2H), 6.74-6.85 (m, 2H), 7.28 (t, 1H), 7.50 (t, 1H), 7.88 (d, 1H), 8.02 (d, 2H), 8.18-8.27 (m, 1H), 8.39-8.55 (m, 4H), 8.87 (t, 1H), 9.52 (s, 1H). |
| 2-2-9 SM = 1-3-7 | 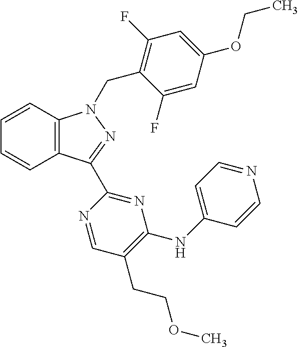 [SMILES: CCOc1cc(F)c(Cn2nc(-c3ncc(CCOC)c(Nc4ccncc4)n3)c3ccccc32)c(F)c1] | 2-[1-(4-ethoxy- 2,6- difluorobenzyl)- 1H-indazol-3-yl]- 5-(2- methoxyethyl)-N- (pyridin-4- yl)pyrimidin-4- amine | ¹H-NMR (300MHz, DMSO- d₆): δ [ppm] = 1.26 (t, 3H), 2.99 (br. t, 2H), 3.29 (s, 3H), 3.60 (t, 2H), 4.03 (q, 2H), 5.70 (s, 2H), 6.79 (d, 2H), 7.20-7.31 (m, 1H), 7.31- 7.42 (m, 2H), 7.49 (br. s., 1H), 7.86 (d, 1H), 8.01-8.12 (m, 2H), 8.37-8.51 (m, 2H), 9.01 (s, 1H) |
| 2-2-10 SM = 1-3-8 The ethyl- ester was cleaved during the reaction | 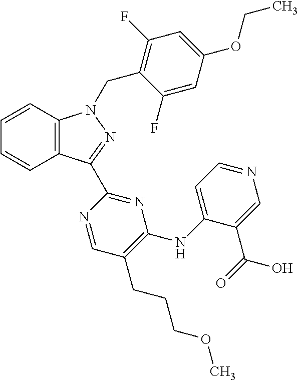 [SMILES: CCOc1cc(F)c(Cn2nc(-c3ncc(CCCOC)c(Nc4ccncc4C(=O)O)n3)c3ccccc32)c(F)c1] | 4- ({2-[1-(4- ethoxy-2,6- difluorobenzyl)- 1H-indazol-3-yl]- 5-(3- methoxypropyl) pyrimidin-4- yl}amino)nicotinic acid | ¹H-NMR (300MHz, DMSO- d₆): δ [ppm] = 1.27 (t, 3H), 1.81-1.95 (m, 2H), 2.73 (br. t, 2H), 3.23 (s, 3H), 3.42 (t, 2H), 4.03 (q, 2H), 5.71 (s, 2H), 6.80 (d, 2H), 7.23-7.35 (m, 1H), 7.45-7.55 (m, 1H), 7.87 (d, 1H), 8.37-8.45 (m, 1H), 8.45-8.55 (m, 2H), 9.00 (s, 1H), 9.36 (d, 1H), 14.20 (br. s, 1H). -NH not detected. |
| 2-2-11 SM = 1-3-8 | 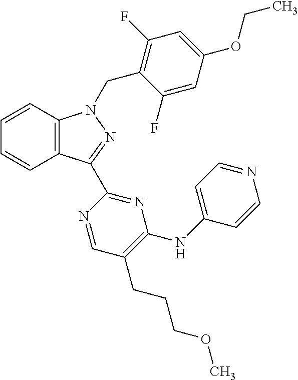 [SMILES: CCOc1cc(F)c(Cn2nc(-c3ncc(CCCOC)c(Nc4ccncc4)n3)c3ccccc32)c(F)c1] | 2-[1-(4-ethoxy- 2,6- difluorobenzyl)- 1H-indazol-3-yl]- 5-(3- methoxypropyl)- N-(pyridin-4- yl)pyrimidin-4- amine | ¹H-NMR (300MHz, DMSO- d₆): δ [ppm] = 1.28 (t, 3H), 1.86 (quin, 2H), 2.77 (t, 2H), 3.25 (s, 3H), 3.38 (t, 2H), 4.03 (q, 2H), 5.69 (s, 2H), 6.72-6.87 (m, 2H), 7.24 (t, 1H), 7.49 (t, 1H), 7.86 (d, 1H), 8.07 (d, 2H), 8.35-8.51 (m, 4H), 8.87 (s, 1H). |
| 2-2-12 SM = 1-3-9 Dioxane instead of DMF | 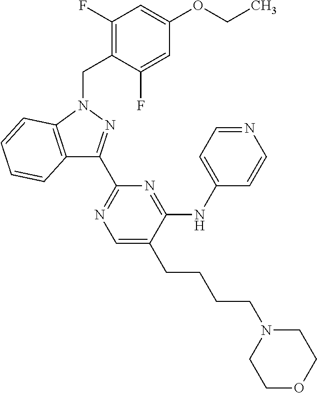 [SMILES: CCOc1cc(F)c(Cn2nc(-c3ncc(CCCCN4CCOCC4)c(Nc4ccncc4)n3)c3ccccc32)c(F)c1] | 2-[1-(4-ethoxy- 2,6- difluorobenzyl)- 1H-indazol-3-yl]- 5-[4-(morpholin-4- yl)butyl]-N- (pyridin-4- yl)pyrimidin-4- amine | ¹H-NMR (300MHz, DMSO- d₆): δ [ppm] = 1.28 (t, 3H), 1.41-1.67 (m, 4H), 2.22- 2.36 (m, 6H), 2.75 (t, 2H), 3.45-3.57 (m, 4H), 4.03 (q, 2H), 5.69 (s, 2H), 6.72-6.83 (m, 2H), 7.24 (t, 1H), 7.48 (ddd, 1H), 7.85 (d, 1H), 8.03-8.14 (m, 2H), 8.31-8.49 (m, 4H), 8.84 (s, 1H). |
| 2-2-13 SM = 1-3-10 Dioxane instead of DMF | 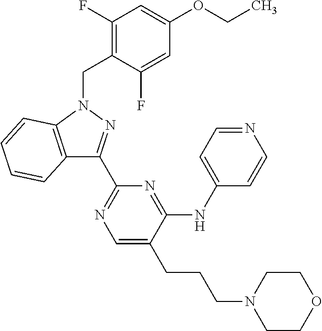 [SMILES: CCOc1cc(F)c(Cn2nc(-c3ncc(CCCN4CCOCC4)c(Nc4ccncc4)n3)c3ccccc32)c(F)c1] | 2-[1-(4-ethoxy- 2,6- difluorobenzyl)- 1H-indazol-3-yl]- 5-[3-(morpholin-4- yl)propyl]-N- (pyridin-4- yl)pyrimidin-4- amine | ¹H-NMR (300MHz, DMSO- d₆): δ [ppm] = 1.28 (m, 3 H) 1.77 (m, 2 H) 2.33 (m, 6 H) 2.75 (m, 2 H) 3.52 (m, 4 H) 4.03 (m, 2 H) 4.03 (d, 2 H) 5.69 (m, 2 H) 6.77 (m, 2 H) 7.24 (t, 1 H) 7.48 (t, 1 H) 7.86 (d, 1 H) 8.08 (d, 2 H) 8.43 (m, 4 H) 8.90 (s, 1 H). |
| 2-2-14 SM = 1-3-10 Dioxane instead of DMF | 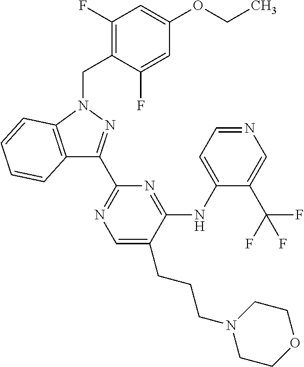 [SMILES: CCOc1cc(F)c(Cn2nc(-c3ncc(CCCN4CCOCC4)c(Nc4ccncc4C(F)(F)F)n3)c3ccccc32)c(F)c1] | 2-[1-(4-ethoxy- 2,6- difluorobenzyl)- 1H-indazol-3-yl]- 5-[3-(morpholin-4- yl)propyl]-N-[3- (trifluoromethyl) pyridin-4- yl]pyrimidin-4- amine | ¹H-NMR (400MHz, DMSO- d₆): δ [ppm] = 1.79 (quin, 2H), 2.24-2.38 (m, 6H), 2.66 (dd, 2H), 3.52 (t, 4H), 4.03 (q, 2H), 5.64 (s, 2H), 6.62-6.80 (m, 2H), 7.06 (t, 1H), 7.33-7.48 (m, 1H), 7.76 (d, 1H), 7.97 (br. s., 1H), 8.13 (br. s., 1H), 8.34 (br. s., 1H), 8.68 (br. s., 1H), 8.83 (br. s., 1H). |
| 2-2-15 SM = 1-3-11 Dioxane instead of DMF | 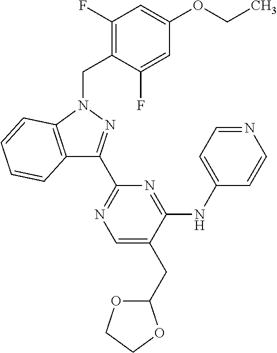 [SMILES: CCOc1cc(F)c(Cn2nc(-c3ncc(CC4OCCO4)c(Nc4ccncc4)n3)c3ccccc32)c(F)c1] | 5-(1,3-dioxolan-2- ylmethyl)-2-[1-(4- ethoxy-2,6- difluorobenzyl)- 1H-indazol-3-yl]- N-(pyridin-4- yl)pyrimidin-4- amine | ¹H-NMR (400MHz, DMSO- d₆): δ [ppm] = 1.28 (t, 3 H) 3.14 (d, 2 H) 3.80 (m, 2 H) 3.89 (m, 2 H) 4.03 (q, 2 H) 5.11 (t, 1 H) 5.70 (s, 2H) 6.80 (m, 2 H) 7.26 (t, 1 H) 7.49 (td, 1 H) 7.86 (d, 1 H) 8.05 (m, 2 H) 8.44 (m, 4 H) 8.98 (s, 1 H). |
| 2-2-16 SM = 1-3-12 Dioxane instead of DMF | 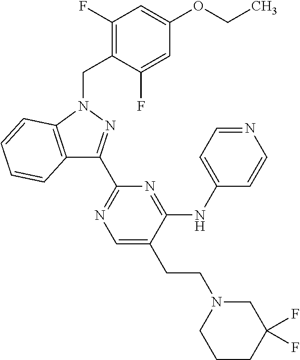 [SMILES: CCOc1cc(F)c(Cn2nc(-c3ncc(CCN4CCCC(F)(F)C4)c(Nc4ccncc4)n3)c3ccccc32)c(F)c1] | 5-[2-(3,3- difluoropiperidin- 1-yl)ethyl]-2-[1-(4- ethoxy-2,6- difluorobenzyl)- 1H-indazol-3-yl]- N-(pyridin-4- yl)pyrimidin-4- amine | ¹H-NMR (300MHz, DMSO- d₆): δ [ppm] = 1.28 (t, 3H), 1.67 (br. s., 2H), 1.79-1.98 (m, 2H), 2,5-2,6 (m, 2H), 2.65-2.74 (m, 2H), 2.79 (t, 2H), 2.86-2.95 (m, 2H), 4.04 (q, 2H), 5.69 (s, 2H), 6.78 (d, 2H), 7.24 (t, 1H), 7.48 (t, 1H), 7.85 (d, 1H), 7.91-8.08 (m, 2H), 8.32- 8.49 (m, 4H), 9.20 (s, 1H). |
| 2-2-17 SM = 1-3-13 Dioxane instead of DMF | 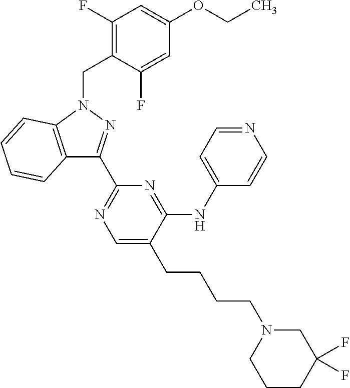 [SMILES: CCOc1cc(F)c(Cn2nc(-c3ncc(CCCCN4CCCC(F)(F)C4)c(Nc4ccncc4)n3)c3ccccc32)c(F)c1] | 5-[4-(3,3- difluoropiperidin- 1-yl)butyl]-2-[1-(4- ethoxy-2,6- difluorobenzyl)- 1H-indazol-3-yl]- N-(pyridin-4- yl)pyrimidin-4- amine | ¹H-NMR (400MHz, DMSO- d₆): δ [ppm] = 1.28 (t, 3H), 1.46-1.55 (m, 2H), 1.55- 1.64 (m, 4H), 1.74-1.89 (m, 2H), 2.31-2.41 (m, 4H), 2.57 (t, 2H), 2.75 (t, 2H), 4.03 (q, 2H), 5.69 (s, 2H), 6.78 (d, 2H), 7.24 (t, 1H), 7.48 (t, 1H), 7.86 (s,1H), 8.07 (d, 2H), 8.32-8.47 (m, 4H), 8.84 (s, 1H). |
| 2-2-18 SM = 1-3-14 Dioxane instead of DMF | 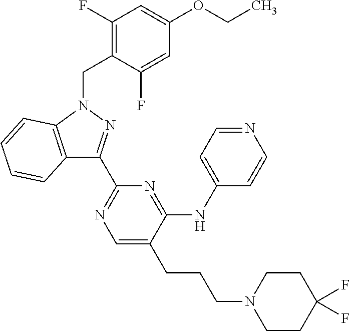 [SMILES: CCOc1cc(F)c(Cn2nc(-c3ncc(CCCN4CCC(F)(F)CC4)c(Nc4ccncc4)n3)c3ccccc32)c(F)c1] | 5-[3-(4,4- difluoropiperidin- 1-yl)propyl]-2-[1- (4-ethoxy-2,6- difluorobenzyl)- 1H-indazol-3-yl]- N-(pyridin-4- yl)pyrimidin-4- amine | ¹H-NMR (400MHz, DMSO- d₆): δ [ppm] = 1.22-1.32 (m, 3H), 1.70-1.83 (m, 2H), 1.83-1.99 (m, 4H), 2.39 (t, 2H), 2.45 (t, 4H), 2.76 (t, 2H), 3.98-4.10 (m, 2H), 5.69 (s, 2H), 6.71-6.83 (m, 2H), 7.24 (t, 1H), 7.48 (td, 1H), 7.85 (d, 1H), 7.98-8.11 (m, 2H), 8.32-8.48 (m, 4H), 8.88 (s, 1H). |
| 2-2-19 SM = 1-3-15 Dioxane instead of DMF | 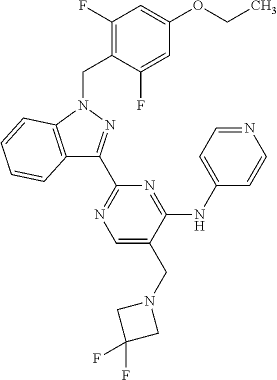 [SMILES: CCOc1cc(F)c(Cn2nc(-c3ncc(CN4CC(F)(F)C4)c(Nc4ccncc4)n3)c3ccccc32)c(F)c1] | 5-[(3,3- difluoroazetidin-1- yl)methyl]-2-[1-(4- ethoxy-2,6- difluorobenzyl)- 1H-indazol-3-yl]- N-(pyridin-4- yl)pyrimidin-4- amine | ¹H-NMR (300MHz, DMSO- d₆): δ [ppm] = 1.28 (t, 3H), 3.74 (t, 4H), 3.91 (s, 2H), 4.04 (q, 2H), 5.70 (s, 2H), 6.79 (d, 2H), 7.28 (t, 1H), 7.50 (t, 1H), 7.87 (d, 1H), 7.96-8.06 (m, 2H), 8.35- 8.56 (m, 4H), 9.45 (s, 1H). |
| 2-2-20 SM = 1-3-16 Dioxane instead of DMF | 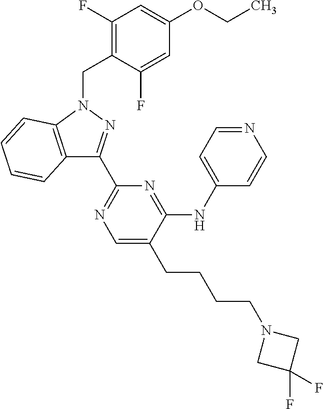 [SMILES: CCOc1cc(F)c(Cn2nc(-c3ncc(CCCCN4CC(F)(F)C4)c(Nc4ccncc4)n3)c3ccccc32)c(F)c1] | 5-[4-(3,3- difluoroazetidin-1- yl)butyl]-2-[1-(4- ethoxy-2,6- difluorobenzyl)- 1H-indazol-3-yl]- N-(pyridin-4- yl)pyrimidin-4- amine | ¹H-NMR (400MHz, DMSO- d₆): δ [ppm] = 1.27 (m, 3 H) 1.39 (m, 2 H) 1.60 (m, 2 H) 2.73 (t, 2 H) 3.16 (d, 2 H) 3.51 (m, 3 H) 4.05 (m, 3 H) 5.69 (s, 2 H) 6.77 (m, 2 H) 7.24 (t, 1 H) 7.38 (m, 1 H) 7.48 (m, 1 H) 7.85 (d, 1 H) 8.07 (m, 2 H) 8.43 (m, 3 H) 8.83 (s, 1 H). |
| 2-2-21 SM = 1-3-17 Dioxane instead of DMF | 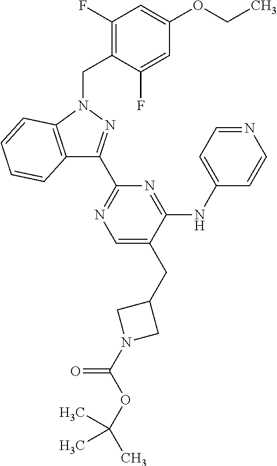 [SMILES: CCOc1cc(F)c(Cn2nc(-c3ncc(CC4CN(C(=O)OC(C)(C)C)C4)c(Nc4ccncc4)n3)c3ccccc32)c(F)c1] | tert-butyl 3-({2-[1- (4-ethoxy-2,6- difluorobenzyl)- 1H-indazol-3-yl]- 4-(eparate-4- ylamino)pyrimidin- 5- yl}methyl) azetidine- 1-carboxylate | ¹H-NMR (400MHz, DMSO- d₆): δ [ppm] = 1.28 (t, 3H), 1.34-1.38 (m, 9H), 2.91 (br. s., 1H), 3.03 (d, 2H), 3.60 (br. s., 2H), 3.94 (s, 2H), 4.03 (q, 2H), 5.69 (s, 2H), 6.78 (d, 2H), 7.25 (t, 1H), 7.40-7.55 (m, 1H), 7.86 (d, 1H), 8.01-8.13 (m, 2H), 8.37-8.53 (m, 4H), 8.92 (s, 1H). |
| 2-2-22 SM = 1-3-20 Dioxane instead of DMF | 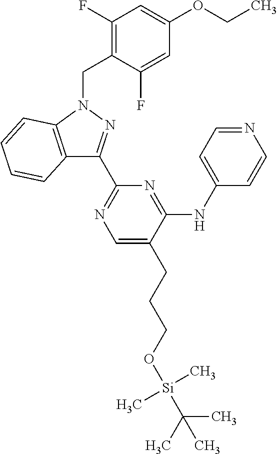 [SMILES: CCOc1cc(F)c(Cn2nc(-c3ncc(CCCO[Si](C)(C)C(C)(C)C)c(Nc4ccncc4)n3)c3ccccc32)c(F)c1] | 5-(3-{[tert- butyl(dimethyl) silyl]oxy} propyl)-2-[1- (4-ethoxy-2,6- difluorobenzyl)- 1H-indazol-3-yl]- N-(pyridin-4- yl)pyrimidin-4- amine | ¹H-NMR (400MHz, DMSO- d₆): δ [ppm] = 0.02 (br. s., 1H), 0.81-0.89 (m, 9H), 1.28 (t, 3H), 1.72-1.87 (m, 2H), 2.51-2.52 (m, 2H), 2.77 (s, 2H), 3.65 (t, 2H), 4.03 (q, 2H), 5.69 (s, 2H), 6.78 (d, 2H), 7.20-7.28 (m, 1H), 7.45-7.52 (m, 1H), 7.85 (d, 1H), 7.99-8.11 (m, 2H), 8.38-8.47 (m, 4H), 8.86 (s, 1H). |
| 2-2-23 SM = 1-3-17 Dioxane instead of DMF | 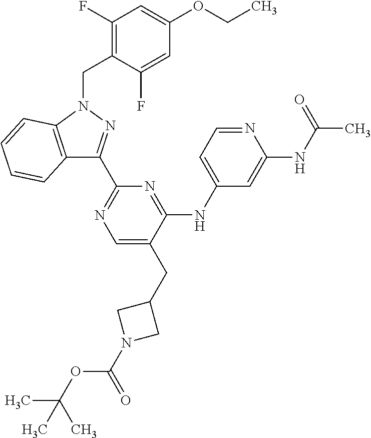 [SMILES: CCOc1cc(F)c(Cn2nc(-c3ncc(CC4CN(C(=O)OC(C)(C)C)C4)c(Nc4ccnc(NC(C)=O)c4)n3)c3ccccc32)c(F)c1] | tert-butyl 3-({4- [(2- acetamidopyridin- 4-yl)amino]-2-[1- (4-ethoxy-2,6- difluorobenzyl)- 1H-indazol-3- yl]pyrimidin-5- yl}methyl) azetidine-1- carboxylate | ¹H NMR (300 MHz, DMSO- d₆) δ ppm 1.16-1.49 (m, 12 H) 2.08 (s, 3 H) 3.04 (d, 2 H) 3.44-3.75 (m, 2 H) 3.77- 4.14 (m, 5 H) 5.68 (s, 2 H) 6.75 (s, 1 H) 6.78 (s, 1 H) 7.20 (t, 1 H) 7.39-7.57 (m, 1 H) 7.83 (d, 1 H) 8.14 (d, 1 H) 8.21-8.34 (m, 2 H) 8.34- 8.48 (m, 2 H) 9.10 (s, 1 H) 10.35 (s, 1 H). |
| 2-2-24 SM = 1-3-17 Dioxane instead of DMF | 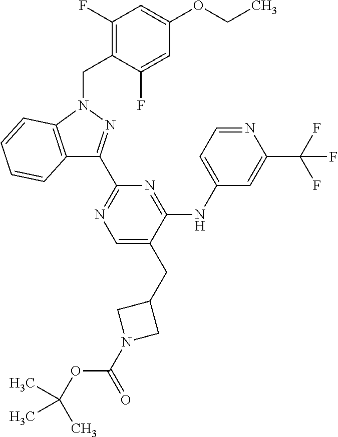 [SMILES: CCOc1cc(F)c(Cn2nc(-c3ncc(CC4CN(C(=O)OC(C)(C)C)C4)c(Nc4ccnc(C(F)(F)F)c4)n3)c3ccccc32)c(F)c1] | tert-butyl 3-[(2-[1- (4-ethoxy-2,6- difluorobenzyl)- 1H-indazol-3-yl]- 4-{[2- (trifluoromethyl) pyridin-4- yl]amino} pyrimidin-5- yl)methyl] azetidine- 1-carboxylate | ¹H NMR (300 MHz, DMSO- d₆) δ ppm 1.29 (t, 3 H) 1.37 (s, 9 H) 2.95 (m, 1 H) 3.07 (d, 2 H) 3.62 (t, 2 H) 3.96 (t, 2 H) 4.04 (q, 2 H) 5.72 (s, 2 H) 6.79 (d, 2 H) 7.26 (t, 1 H) 7.51 (dt, 1 H) 7.85 (d, 1 H) 8.44 (d, 1 H) 8.49 (s, 1 H) 8.54 (s, 1 H) 8.60 (s, 2 H) 9.30 (s, 1 H). |
| 2-2-25 SM = 1-3-17 Dioxane instead of DMF | 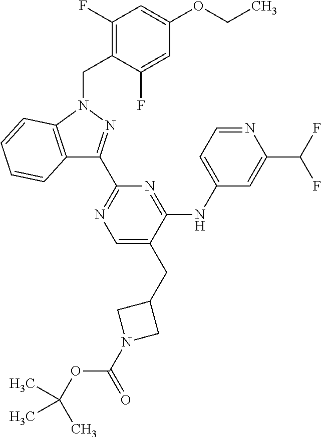 [SMILES: CCOc1cc(F)c(Cn2nc(-c3ncc(CC4CN(C(=O)OC(C)(C)C)C4)c(Nc4ccnc(C(F)F)c4)n3)c3ccccc32)c(F)c1] | tert-butyl 3-[(4- {[2-(difluoromethyl) pyridin-4-yl]amino}- 2-[1-(4-ethoxy- 2,6- difluorobenzyl)- 1H-indazol-3- yl]pyrimidin-5- yl)methyl] azetidine- 1-carboxylate | ¹H NMR (300 MHz, DMSO- d₆) δ ppm 1.29 (t, 3 H) 1.37 (m, 9 H) 2.90-2.95 (m, 1 H) 3.06 (d, 2 H) 3.62 (s, 2 H) 3.83-4.13 (m, 4 H) 5.72 (s, 2 H) 6.78 (s, 1 H) 6.80 (s, 1 H) 6.91 (t, 1 H) 7.26 (t, 1 H) 7.44-7.54 (m, 1 H) 7.85 (d, 1 H) 8.17 (d, 1 H) 8.45 (d, 1 H) 8.48-8.65 (m, 3 H) 9.19 (s, 1 H). |
| 2-2-26 SM = 1-3-5 Dioxane instead of DMF | 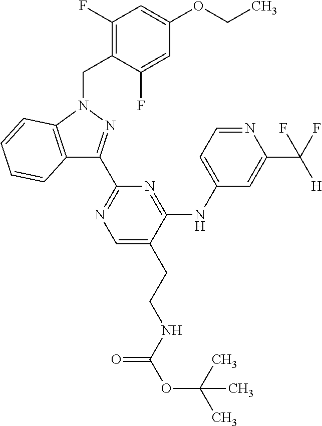 [SMILES: CCOc1cc(F)c(Cn2nc(-c3ncc(CCNC(=O)OC(C)(C)C)c(Nc4ccnc(C(F)F)c4)n3)c3ccccc32)c(F)c1] | tert-butyl [2-(4- {[2- (difluoromethyl) pyridin-4-yl]amino}- 2-[1-(4-ethoxy- 2,6- difluorobenzyl)- 1H-indazol-3- yl]pyrimidin-5- yl)ethyl]carbamate | ¹H-NMR (400MHz, DMSO- d₆): δ [ppm] = 1.27 (t, 3 H) 1.33 (s, 9 H) 2.85 (t, 2 H) 3.18 (m, 2 H) 4.02 (m, 2 H) 5.71 (s, 2 H) 6.77 (m, 2 H) 7.18 (t, 1 H) 7.25 (t, 1 H) 7.48 (t, 1 H) 7.84 (d, 1 H) 8.15 (s, 1 H) 8.42 (m, 2 H) 8.51 (d, 1 H) 8.62 (d, 1 H) 9.39 (s, 1 H). |
| 2-2-27 SM = 1-3-5 Dioxane instead of DMF | 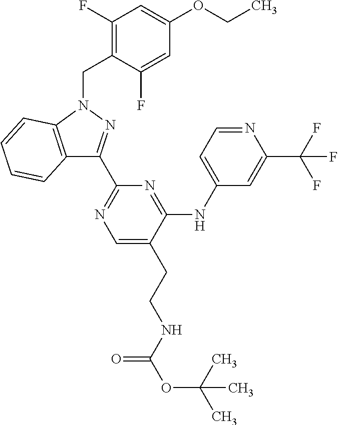 [SMILES: CCOc1cc(F)c(Cn2nc(-c3ncc(CCNC(=O)OC(C)(C)C)c(Nc4ccnc(C(F)(F)F)c4)n3)c3ccccc32)c(F)c1] | tert-butyl [2-(2-[1- (4-ethoxy-2,6- difluorobenzyl)- 1H-indazol-3-yl]- 4-{[2- (trifluoromethyl) pyridin-4- yl]amino} pyrimidin-5- yl)ethyl]carbamate | ¹H-NMR (400MHz, DMSO- d₆): δ [ppm] = 1.29 (t, 3H), 1.34 (s, 9H), 2.88 (t, 2H), 3.15-3.27 (m, 2H), 4.04 (q, 2H), 5.73 (s, 2H), 6.80 (d, 2H), 7.19 (t, 1H), 7.26 (t, 1H), 7.45-7.57 (m, 1H), 7.85 (d, 1H), 8.45 (d, 3H), 8.60 (d, 1H), 8.66 (d, 1H), 9.52 (s, 1H). |
| 2-2-28 SM = 1-3-5 Dioxane instead of DMF | 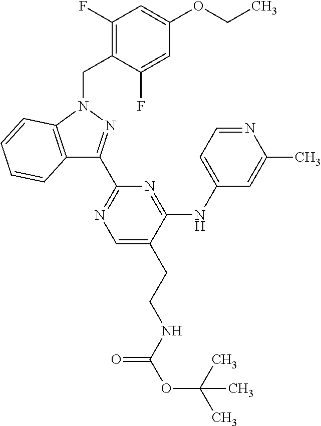 [SMILES: CCOc1cc(F)c(Cn2nc(-c3ncc(CCNC(=O)OC(C)(C)C)c(Nc4ccnc(C)c4)n3)c3ccccc32)c(F)c1] | tert-butyl (2-{2-[1- (4-ethoxy-2,6- difluorobenzyl)- 1H-indazol-3-yl]- 4-[(2- methylpyridin-4- yl)amino] pyrimidin-5- yl}ethyl)carbamate | ¹H-NMR (400MHz, DMSO- d₆): δ [ppm] = 1.27 (t, 3 H) 1.35 (m, 12H) 2.83 (t, 2 H) 3.18 (m, 2 H) 4.02 (q, 2 H) 5.70 (s, 2 H) 6.76 (d, 2 H) 7.15 (t, 1 H) 7.25 (t, 1 H) 7.48 (t, 1 H) 7.84 (d, 1 H) 7.93 (m, 2 H) 8.29 (d, 1 H) 8.34 (s, 1 H) 8.46 (d, 1 H) 9.01 (s, 1 H). |
| 2-2-29 SM = 1-3-5 | 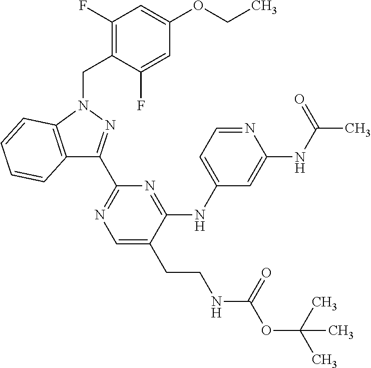 [SMILES: CCOc1cc(F)c(Cn2nc(-c3ncc(CCNC(=O)OC(C)(C)C)c(Nc4ccnc(NC(C)=O)c4)n3)c3ccccc32)c(F)c1] | tert-butyl [2-(4- {[2- (acetylamino) pyridin-4-yl]amino}- 2-[1-(4-ethoxy-2,6- difluorobenzyl)- 1H-indazol-3- yl]pyrimidin-5- yl)ethyl]carbamate | ¹H NMR (400 MHz, DMSO- d₆) δ ppm 1.19-1.48 (m, 12 H) 2.07 (s, 3 H) 2.77-2.95 (m, 2 H) 3.16-3.22 (m, 2 H) 4.02 (q, 2 H) 5.68 (s, 2 H) 6.75 (s, 1 H) 6.78 (s, 1 H) 6.81-7.05 (m, 1 H) 7.05- 7.23 (m, 1 H) 7.23-7.60 (m, 1 H) 7.84 (t, 1 H) 8.13 (d, 1 H) 8.19-8.34 (m, 2 H) 8.42 (d, 1 H) 9.14 (s, 1 H) 10.32 (s, 1 H). |
| 2-2-30 SM = 1-3-18 Dioxane instead of DMF | 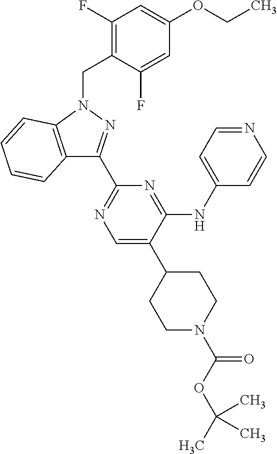 [SMILES: CCOc1cc(F)c(Cn2nc(-c3ncc(C4CCN(C(=O)OC(C)(C)C)CC4)c(Nc4ccncc4)n3)c3ccccc32)c(F)c1] | tert-butyl 4-{2-[1- (4-ethoxy-2,6- difluorobenzyl)- 1H-indazol-3-yl]- 4-(pyridin-4- ylamino)pyrimidin- 5-yl}piperidine-1- carboxylate | ¹H-NMR (400MHz, DMSO- d₆): δ [ppm] = 1.29 (t, 3H), 1.44 (s, 9H), 1.51-1.67 (m, 2H), 1.86 (d, 2H), 2.75-3.06 (m, 2H), 3.13-3.29 (m, 1H), 4.04 (q, 2H), 4.14 (d, 2H), 5.70 (s, 2H), 6.79 (m, 2H), 7.14-7.31 (m, 1H), 7.50 (ddd, 1H), 7.86 (d, 1H),7.95- 8.11 (m, 2H), 8.35-8.48 (m, 3H), 8.53 (s, 1H), 8.97 (s, 1H). |
| 2-2-31 SM = 1-3-18 | 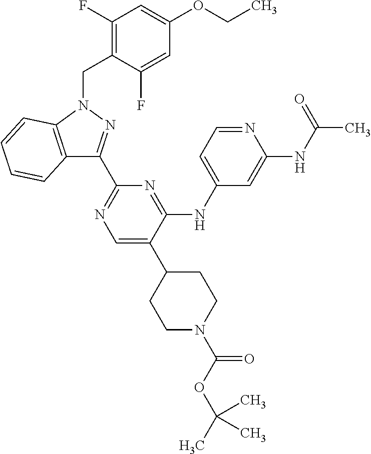 [SMILES: CCOc1cc(F)c(Cn2nc(-c3ncc(C4CCN(C(=O)OC(C)(C)C)CC4)c(Nc4ccnc(NC(C)=O)c4)n3)c3ccccc32)c(F)c1] | tert-butyl 4-(4-{[2- (acetylamino) pyridin-4-yl] amino}-2- [1-(4-ethoxy-2,6- difluorobenzyl)- 1H-indazol-3- yl]pyrimidin-5- yl)piperidine-1- carboxylate | ¹H-NMR (400MHz, DMSO- d₆): δ [ppm] = 1.29 (t, 3H), 1.44 (s, 9H), 1.50-1.66 (m, 2H), 1.78-1.91 (m, 2H), 2.10 (s, 3H), 2.81-3.06 (m, 2H), 3.21-3.31 (m, 1H), 4.11 (q, 4H), 5.69 (s, 2H), 6.71-6.83 (m, 2H), 7.20 (t, 1H), 7.48 (t, 1H), 7.83 (d, 1H), 8.15 (s, 2H), 8.23 (s, 1H), 8.40 (d, 1H), 8.52 (s, 1H), 9.16 (s, 1H), 10.37 (s, 1H). |
| 2-2-32 SM = 1-3-21 | 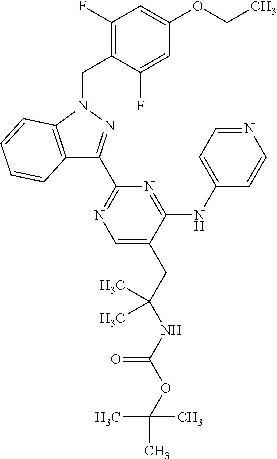 [SMILES: CCOc1cc(F)c(Cn2nc(-c3ncc(CC(C)(C)NC(=O)OC(C)(C)C)c(Nc4ccncc4)n3)c3ccccc32)c(F)c1] | tert-butyl (1-{2-[1- (4-ethoxy-2,6- difluorobenzyl)- 1H-indazol-3-yl]- 4-(pyridin-4- ylamino)pyrimidin- 5-yl}-2- methylpropan-2- yl)carbamate | ¹H-NMR (400MHz, DMSO- d₆): δ [ppm] = 1.23 (s, 6H), 1.29 (t, 3H), 1.42 (s, 9H), 3.16 (s, 2H), 4.05 (q, 2H), 5.72 (s, 2H), 6.62 (br. s., 1H), 6.81 (d, 2H), 7.27 (t, 1H), 7.51 (t, 1H), 7.88 (d, 1H), 8.06 (d, 2H), 8.27 (s, 1H), 8.37-8.51 (m, 3H), 8.94 (s, 1H). |
| 2-2-33 SM = 1-3-18 | 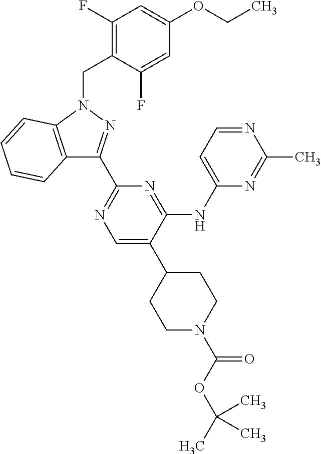 [SMILES: CCOc1cc(F)c(Cn2nc(-c3ncc(C4CCN(C(=O)OC(C)(C)C)CC4)c(Nc4ccnc(C)n4)n3)c3ccccc32)c(F)c1] | tert-butyl 4-{2-[1- (4-ethoxy-2,6- difluorobenzyl)- 1H-indazol-3-yl]- 4-[(2- methylpyrimidin- 4- yl)amino]pyrimidin- 5-yl}piperidine- 1-carboxylate | ¹H-NMR (400MHz, DMSO- d₆): δ [ppm] = 1.29 (t, 3H), 1.44 (s, 9H), 1.59 (d, 2H), 1.82 (d, 2H), 2.58 (s, 3H), 2.83-3.02 (m, 2H), 3.34- 3.42 (m, 1H), 4.04 (q, 2H), 4.08-4.16 (m, 2H), 5.71 (s, 2H), 6.76-6.84 (m, 2H), 7.28 (t, 1H), 7.47-7.54 (m, 1H), 7.87 (d, 1H), 8.43-8.51 (m, 3H), 8.62 (s, 1H), 9.82 (s, 1H). |
| 2-2-34 SM = 1-3-18 | 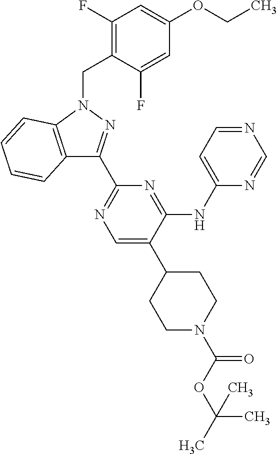 [SMILES: CCOc1cc(F)c(Cn2nc(-c3ncc(C4CCN(C(=O)OC(C)(C)C)CC4)c(Nc4ccncn4)n3)c3ccccc32)c(F)c1] | tert-butyl 4-{2-[1- (4-ethoxy-2,6- difluorobenzyl)- 1H-indazol-3-yl]- 4-(pyrimidin-4- ylamino)pyrimidin- 5-yl}piperidine-1- carboxylate | ¹H-NMR (400MHz, DMSO- d₆): δ [ppm] = 1.29 (t, 3H), 1.44 (s, 9H), 1.51-1.66 (m, 2H), 1.78-1.87 (m, 2H), 2.81-3.01 (m, 2H), 3.35- 3.41 (m, 1H), 4.00-4.17 (m, 4H), 5.71 (s, 2H), 6.78-6.85 (m, 2H), 7.25-7.31 (m, 1H), 7.48-7.54 (m, 1H), 7.88 (d, 1H), 8.46 (d, 1H), 8.57 (d, 1H), 8.63-8.71 (m, 2H), 8.89 (d, 1H), 9.93 (s, 1H). |
| 2-2-35 SM = 1-3-19 | 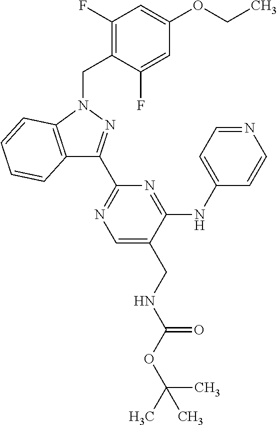 [SMILES: CCOc1cc(F)c(Cn2nc(-c3ncc(CNC(=O)OC(C)(C)C)c(Nc4ccncc4)n3)c3ccccc32)c(F)c1] | tert-butyl ({2-[1- (4-ethoxy-2,6- difluorobenzyl)- 1H-indazol-3-yl]- 4-(pyridin-4- ylamino)pyrimidin- 5-yl}methyl) carbamate | ¹H-NMR (400MHz, DMSO- d₆): δ [ppm] = 1.30 (t, 3H), 1.38-1.45 (m, 9H), 4.05 (q, 2H), 4.28 (d, 2H), 5.72 (s, 2H), 6.74-6.88 (m, 2H), 7.29 (t, 1H), 7.33-7.44 (m, 1H), 7.52 (ddd, 1H), 7.65- 7.79 (m, 1H), 7.89 (d, 1H), 8.04 (d, 2H), 8.36-8.53 (m, 3H), 9.37 (s, 1H). |
| 2-2-36 SM = 1-3-19 | 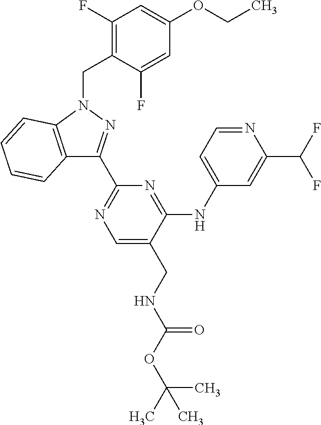 [SMILES: CCOc1cc(F)c(Cn2nc(-c3ncc(CNC(=O)OC(C)(C)C)c(Nc4ccnc(C(F)F)c4)n3)c3ccccc32)c(F)c1] | tert-butyl [(4-{[2- (difluoromethyl) pyridin-4-yl]amino}- 2-[1-(4-ethoxy- 2,6- difluorobenzyl)- 1H-indazol-3- yl]pyrimidin-5- yl)methyl] carbamate | LC-MS (Method 6): retention time: 1.57 min MS ES+: 638 [M + H]⁺ |
| 2-2-37 SM = 1-3-18 | 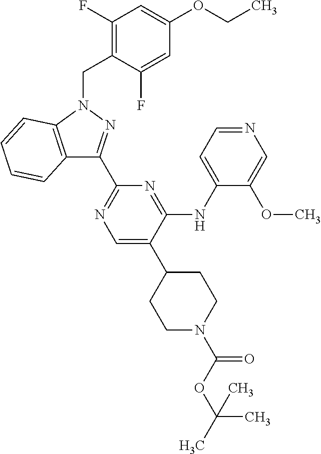 [SMILES: CCOc1cc(F)c(Cn2nc(-c3ncc(C4CCN(C(=O)OC(C)(C)C)CC4)c(Nc4ccncc4OC)n3)c3ccccc32)c(F)c1] | tert-butyl 4-{2-[1- (4-ethoxy-2,6- difluorobenzyl)- 1H-indazol-3-yl]- 4-[(3- methoxypyridin-4- yl)amino]pyrimidin- 5-yl}piperidine- 1-carboxylate | ¹H-NMR (400MHz, DMSO- d₆): δ [ppm] = 1.28 (t, 3H), 1.43 (s, 10H), 1.54-1.67 (m, 2H), 1.86-1.95 (m, 2H), 2.86-3.02 (m, 2H), 3.96 (s, 3H), 4.03 (q, 2H), 4.09- 4.22 (m, 2H), 5.67 (s, 2H), 6.74-6.82 (m, 2H), 7.17- 7.23 (m, 1H), 7.43-7.50 (m, 1H), 7.83 (d, 1H), 8.12 (s, 1H), 8.19 (d, 1H), 8.30 (d, 1H), 8.37 (s, 1H), 8.47 (s, 1H), 8.54 (d, 1H). |
| 2-2-38 SM = 1-3-18 | 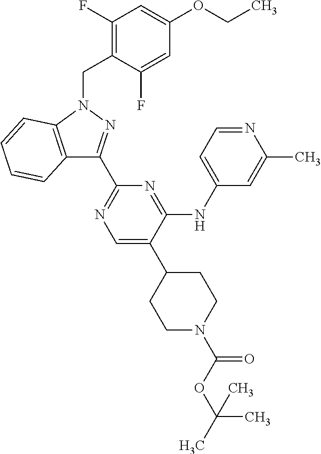 [SMILES: CCOc1cc(F)c(Cn2nc(-c3ncc(C4CCN(C(=O)OC(C)(C)C)CC4)c(Nc4ccnc(C)c4)n3)c3ccccc32)c(F)c1] | tert-butyl 4-{2-[1- (4-ethoxy-2,6- difluorobenzyl)- 1H-indazol-3-yl]- 4-[(2- methylpyridin-4- yl)amino]pyrimidin- 5-yl}piperidine- 1-carboxylate | ¹H-NMR (400MHz, DMSO- d₆): δ [ppm] = 1.29 (t, 3H), 1.44 (s, 9H), 1.51-1.65 (m, 2H), 1.81-1.90 (m, 2H), 2.45 (s, 3H), 2.85-3.00 (m, 2H), 3.17-3.27 (m, 1H), 3.99-4.08 (m, 2H), 4.09- 4.20 (m, 2H), 5.71 (s, 2H), 6.73-6.83 (m, 2H), 7.21- 7.28 (m, 1H), 7.46-7.52 (m, 1H), 7.82-7.89 (m, 3H), 8.32 (d, 1H), 8.44 (d, 1H), 8.51 (s, 1H), 8.88 (s, 1H). |
| 2-2-39 SM = 1-3-18 | 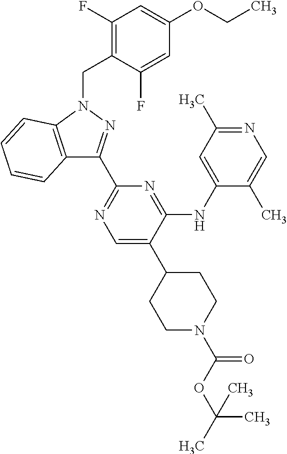 [SMILES: CCOc1cc(F)c(Cn2nc(-c3ncc(C4CCN(C(=O)OC(C)(C)C)CC4)c(Nc4cc(C)ncc4C)n3)c3ccccc32)c(F)c1] | tert-butyl 4-{4- [(2,5- dimethylpyridin-4- yl)aminol-2-[1-(4- ethoxy-2,6- difluorobenzyl)- 1H-indazol-3- yl]pyrimidin-5- yl}piperidine-1- carboxylate | LC-MS (Method 5): retention time: 1.54 min MS ES+: 671 [M + H]⁺ |
| 2-2-40 SM = 1-3-22 | 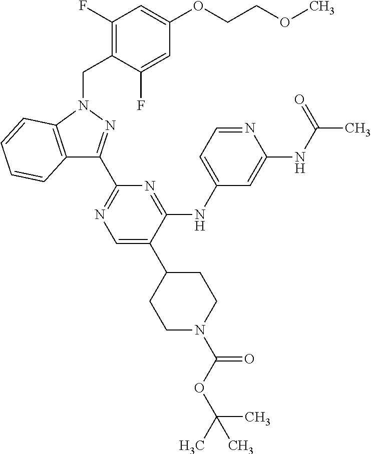 [SMILES: COCCOc1cc(F)c(Cn2nc(-c3ncc(C4CCN(C(=O)OC(C)(C)C)CC4)c(Nc4ccnc(NC(C)=O)c4)n3)c3ccccc32)c(F)c1] | tert-butyl 4-(4-[(2- acetamidopyridin- 4-yl)amino]-2-{1- [2,6-difluoro-4-(2- methoxyethoxy) benzyl]-1H- indazol-3- yl}pyrimidin-5- yl)piperidine-1- carboxylate | ¹H-NMR (400MHz, DMSO- d₆): δ [ppm] = 1.43 (s, 9H), 1.56 (m, 2H), 1.84 (br. d., 2H), 2.09 (s, 3H), 2.54 (m, 1H), 2.91 (m, 2H), 3.26 (s, 3H), 3.61 (m, 2H), 4.11 (m, 2H), 4.13 (m, 2H), 5.69 (s, 2H), 6.81 (m, 2H), 7.19 (dd, 1H), 7.47 (dd, 1H), 7.82 (d, 1H), 8.15 (m, 2H), 8.23 (s, 1H), 8.40 (d, 1H), 8.51 (s, 1H), 9.15 (br. s.,1H), 10.36 (br. s., 1H). |
| 2-2-41 SM = 1-3-22 | 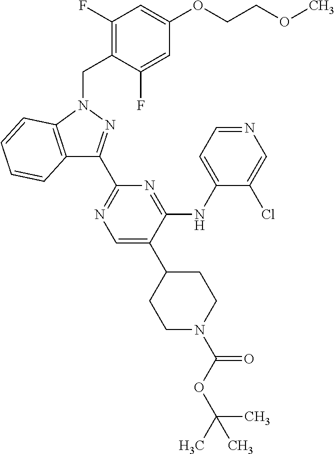 [SMILES: COCCOc1cc(F)c(Cn2nc(-c3ncc(C4CCN(C(=O)OC(C)(C)C)CC4)c(Nc4ccncc4Cl)n3)c3ccccc32)c(F)c1] | tert-butyl 4-(4-[(3- chloropyridin-4- yl)amino]-2-{1- [2,6-difluoro-4-(2- methoxyethoxy) benzyl]-1H- indazol-3- yl}pyrimidin-5- yl)piperidine-1- carboxylate | ¹H-NMR (400 MHz, DMSO- d₆): δ [ppm] = 1.43 (s, 9H), 1.61 (m, 2H), 1.94 (br. d., 2H), 2.90 (m, 1H), 3.07 (m, 2H), 3.26 (s, 3H), 3.61 (m, 2H), 4.11 (m, 2H), 4.15 (m, 2H), 5.66 (s, 2H), 6.81 (m, 2H), 7.08 (dd, 1H), 7.43 (dd, 1H), 7.80 (d, 1H), 7.99 (d, 1H), 8.22 (d, 1H), 8.50 (s, 1H), 8.51 (d, 1H), 8.65 (s, 1H), 8.68 (s, 1H). |
| 2-2-42 SM = 1-3-22 | 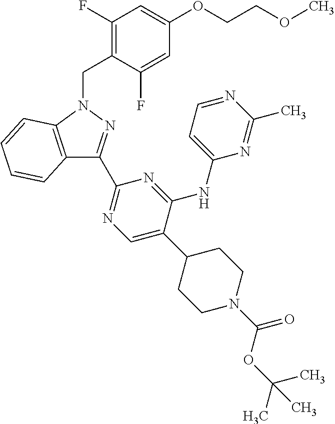 [SMILES: COCCOc1cc(F)c(Cn2nc(-c3ncc(C4CCN(C(=O)OC(C)(C)C)CC4)c(Nc4ccnc(C)n4)n3)c3ccccc32)c(F)c1] | tert-butyl 4-(2-{1- [2,6-difluoro-4-(2- methoxyethoxy) benzyl]-1H- indazol-3-yl}-4- [(2- methylpyrimidin-4- yl)amino] pyrimidin-5- yl)piperidine- 1-carboxylate | ¹H-NMR (400MHz, DMSO- d₆): δ [ppm] = 1.43 (s, 9H), 1.56 (m, 2H), 1.81 (br. d., 2H), 2.57 (s, 3H), 2.91 (m, 1H), 3.26 (s, 3H), 3.37 (m, 2H), 3.61 (m, 2H), 4.10 (m, 2H), 4.11 (m, 2H), 5.71 (s, 2H), 6.83 (m, 2H), 7.27 (dd, 1H), 7.50 (dd, 1H), 7.87 (d, 1H), 8.45 (d, 1H), 8.45 (m, 2H), 8.61 (s, 1H), 9.81 (s, 1H). |
| 2-2-43 SM = 1-3-17 | 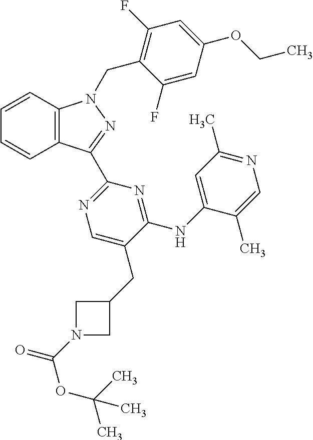 [SMILES: CCOc1cc(F)c(Cn2nc(-c3ncc(CC4CN(C(=O)OC(C)(C)C)C4)c(Nc4cc(C)ncc4C)n3)c3ccccc32)c(F)c1] | tert-butyl 3-({4- [(2,5- dimethylpyridin-4- yl)amino]-2-[1-(4- ethoxy-2,6- difluorobenzyl)- 1H-indazol-3- yl]pyrimidin-5- yl}methyl)azetidine- 1-carboxylate | ¹H-NMR (400MHz, DMSO- d₆): δ [ppm] = 1.30 (t, 3H), 1.37 (s, 9H), 2.16 (s, 3H), 2.46 (s, 3H), 2.99 (br. s., 3H), 3.56-3.69 (m, 2H), 3.91-4.15 (m, 4H), 5.64 (s, 2H), 6.70-6.81 (m, 2H), 7.01-7.08 (m, 1H), 7.38- 7.46 (m, 1H), 7.52 (s, 1H), 7.77 (d, 1H), 7.94-8.01 (m, 1H), 8.34 (d, 2H), 8.46 (s, 1H). |
| 2-2-44 SM = 1-3-17 | 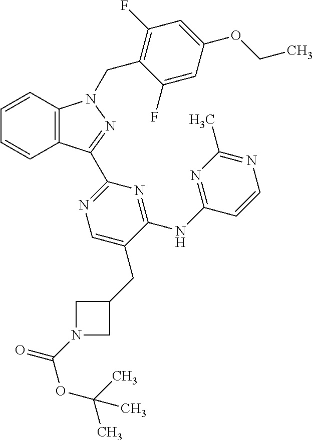 [SMILES: CCOc1cc(F)c(Cn2nc(-c3ncc(CC4CN(C(=O)OC(C)(C)C)C4)c(Nc4ccnc(C)n4)n3)c3ccccc32)c(F)c1] | tert-butyl 3-({2-[1- (4-ethoxy-2,6- difluorobenzyl)- 1H-indazol-3-yl]- 4-[(2- methylpyrimidin- 4- yl)amino] pyrimidin-5- yl}methyl)azetidine- 1-carboxylate | ¹H-NMR (400MHz, DMSO- d₆): δ [ppm] = 1.29 (t, 3H), 1.36 (s, 9H), 2.58 (s, 3H), 2.82-2.92 (m, 1H), 3.10 (br. d, 2H), 3.53-3.64 (m, 2H), 3.91 (br. t, 2H), 4.04 (q, 2H), 5.71 (s, 2H), 6.77- 6.85 (m, 2H), 7.29 (t, 1H), 7.51 (t, 1H), 7.88 (d, 1H), 8.48 (t, 2H), 8.54 (s, 1H), 8.59 (d, 1H), 9.72 (s, 1H). |
| 2-2-45 SM = 1-3-5 | 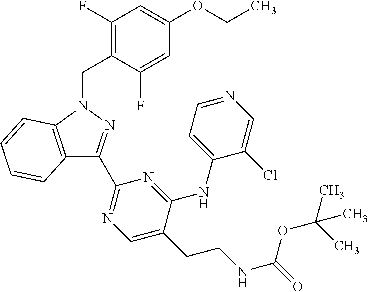 [SMILES: CCOc1cc(F)c(Cn2nc(-c3ncc(CCNC(=O)OC(C)(C)C)c(Nc4ccncc4Cl)n3)c3ccccc32)c(F)c1] | tert-butyl (2-{4- [(3-chloropyridin- 4-yl)amino]-2-[1- (4-ethoxy-2,6- difluorobenzyl)- 1H-indazol-3- yl]pyrimidin-5- yl}ethyl)carbamate | ¹H-NMR (400 MHz, DMSO- d₆): δ [ppm] = 1.29 (t, 3H), 1.29 (s, 9H), 2.86 (t, 2H), 3.28 (td, 2H), 4.03 (q, 2H), 5.68 (s, 2H), 6.79 (m, 2H), 7.04 (br. t., 1H), 7.16 (dd, 1H), 7.46 (dd, 1H), 7.82 (d, 1H), 8.18 (d, 1H), 8.42 (m, 2H), 8.46 (m, 2H), 8.66 (s, 1H). |
| 2-2-46 SM = 1-3-5 | 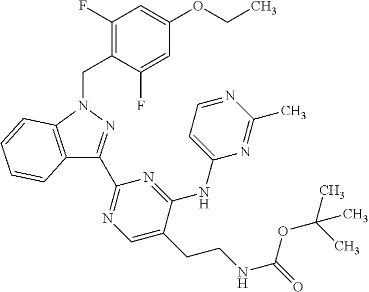 [SMILES: CCOc1cc(F)c(Cn2nc(-c3ncc(CCNC(=O)OC(C)(C)C)c(Nc4ccnc(C)n4)n3)c3ccccc32)c(F)c1] | tert-butyl (2-{2-[1- (4-ethoxy-2,6- difluorobenzyl)- 1H-indazol-3-yl]- 4-[(2- methylpyrimidin-4- yl)amino] pyrimidin-5- yl}ethyl)carbamate | ¹H-NMR (400 MHz, DMSO- d₆): δ [ppm] = 1.27 (s, 9H), 1.28 (t, 3H), 2.56 (s, 3H), 2.91 (t, 2H), 3.18 (td, 2H), 4.03 (q, 2H), 5.71 (s, 2H), 6.81 (m, 2H), 6.88 (br. t., 1H), 7.28 (dd, 1H), 7.49 (dd, 1H), 7.87 (d, 1H), 8.42 (s, 1H), 8.46 (d, 1H), 8.47 (d, 1H), 8.54 (d, 1H), 9.58 (s, 1H). |
| 2-2-47 SM = 1-12-36 | 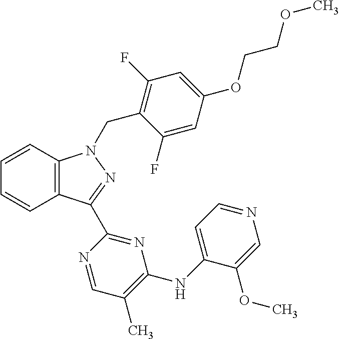 [SMILES: COCCOc1cc(F)c(Cn2nc(-c3ncc(C)c(Nc4ccncc4OC)n3)c3ccccc32)c(F)c1] | 2-{1-[2,6-difluoro- 4-(2- methoxyethoxy) benzyl]-1H- indazol-3-yl}-N- (3- methoxypyridin-4- yl)-5- methylpyrimidin- 4-amine | ¹H-NMR (400MHz, DMSO- d₆): δ [ppm] = 2.30 (s, 3H), 3.26 (s, 3H), 3.57-3.64 (m, 2H), 4.00 (s, 3H), 4.07- 4.16 (m, 2H), 5.71 (s, 2H), 6.80-6.90 (m, 2H), 7.21- 7.28 (m, 1H), 7.45-7.54 (m, 1H), 7.82-7.92 (m, 2H), 8.21 (d, 1H), 8.36-8.42 (m, 2H), 8.46 (d, 1H), 8.75 (d, 1H). |
| 2-2-48 SM = 1-12-38 | 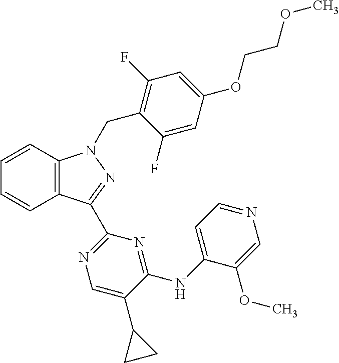 [SMILES: COCCOc1cc(F)c(Cn2nc(-c3ncc(C4CC4)c(Nc4ccncc4OC)n3)c3ccccc32)c(F)c1] | 5-cyclopropyl-2- {1-[2,6-difluoro-4- (2- methoxyethoxy) benzyl]-1H- indazol-3-yl}-N- (3- methoxypyridin-4- yl)pyrimidin-4- amine | ¹H-NMR (400MHz, DMSO- d₆): δ [ppm] = ] = 0.71-0.80 (m, 2H), 1.03-1.12 (m, 2H), 1.85-1.95 (m, 1H), 3.26 (s, 3H), 3.57-3.66 (m, 2H), 4.06 (s, 3H), 4.09-4.17 (m, 2H), 5.72 (s, 2H), 6.80- 6.91 (m, 2H), 7.29 (t, 1H), 7.51 (t, 1H), 7.89 (d, 1H), 8.22 (d, 1H), 8.32-8.41 (m, 2H), 8.43-8.49 (m, 2H), 8.96 (d, 1H). |

### Example 2-3-1

### Preparation of 2-[1-(4-ethoxy-2,6-difluorobenzyl)-1H-indazol-3-yl]-5-[2-(methylsulfonyl)ethyl]-N-(eparate-4-yl)pyrimidin-4-amine

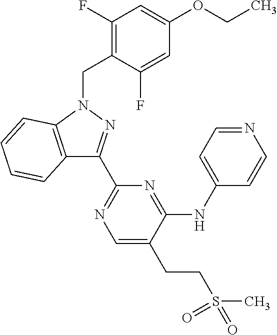 [SMILES: CCOc1cc(F)c(Cn2nc(-c3ncc(CCS(C)(=O)=O)c(Nc4ccncc4)n3)c3ccccc32)c(F)c1]

355 mg of 2-[1-(4-ethoxy-2,6-difluorobenzyl)-1H-indazol-3-yl]-5-[2-(methylsulfanyl)ethyl]-N-(eparate-4-yl)pyrimidin-4-amine 2-2-1 (90%, 0.6 mmol, 1.0 eq.) were dissolved in 1.6 mL chloroform and cooled to 0° C. At this temperature 296 mg of 3-chlorobenzenecarboperoxoic acid (77%, 1.32 mmol, 2.2 eq.), dissolved in 1.5 mL chloroform, were added dropwise. The reaction mixture stirred over night at rt. The reaction mixture was diluted with DCM. The yellow precipitate was filtered off under vacuo and the filter cake was washed with DCM. The filtrate was quenched with 10%-aqueous sodium-thiosulfate-solution. The layers were eparatel and the aqueous layer was extracted with DCM twice. The combined organic layers were washed with saturated sodiumhydrogencarbonate-solution and dried using a waterresistant filter. The filtrate was concentrated under reduced pressure under vacuo. The filter cake and the crude product were combined. It was purified by flash chromatography to provide the 90% pure target compound: 171 mg, 0.27 mmol, 46%.

¹H-NMR (300 MHz, DMSO-d₆): δ [ppm]=1.29 (t, 3H), 3.08 (s, 3H), 3.14-3.27 (m, 2H), 3.49-3.66 (m, 2H), 4.04 (q, 2H), 5.71 (s, 2H), 6.74-6.84 (d, 2H), 7.27 (t, 1H), 7.50 (t, 1H), 7.82-7.90 (m, 1H), 7.99-8.15 (m, 2H), 8.39-8.49 (m, 3H), 8.51 (s, 1H), 9.04 (s, 1H).

The following compounds were prepared according to the same procedure from the indicated starting materials (SM=starting material):

| | | | |
|---|---|---|---|
| 2-3-2 SM = 2-2-2 | 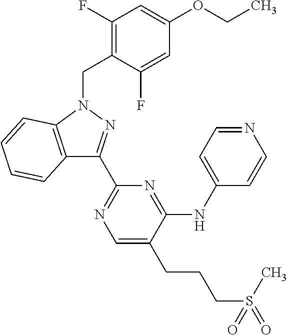 [SMILES: CCOc1cc(F)c(Cn2nc(-c3ncc(CCCS(C)(=O)=O)c(Nc4ccncc4)n3)c3ccccc32)c(F)c1] | 2-[1-(4-ethoxy- 2,6- difluorobenzyl)- 1H-indazol-3-yl]- 5-[3- (methylsulfonyl) propyl]-N- (pyridin- 4-yl)pyrimidin-4- amine | ¹H-NMR (600MHz, DMSO-d₆): δ [ppm] = 1.29 (t, 3H), 2.04 (quin, 2H), 2.89 (t, 2H), 2.98 (s, 3H), 3.19 (t, 2H), 4.04 (q, 2H), 5.70 (s, 2H), 6.75-6.81 (m, 2H), 7.26 (t, 1H), 7.50 (t, 1H), 7.87 (d, 1H), 8.03-8.18 (m, 2H), 8.40-8.54 (m, 4H), 8.89 (s, 1H). |
| 2-3-3 SM = 2-2-3 | 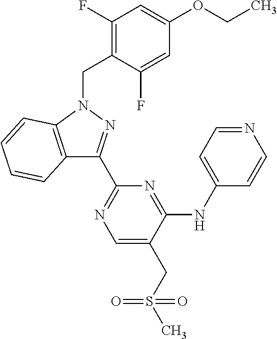 [SMILES: CCOc1cc(F)c(Cn2nc(-c3ncc(CS(C)(=O)=O)c(Nc4ccncc4)n3)c3ccccc32)c(F)c1] | 2-[1-(4-ethoxy- 2,6- difluorobenzyl)- 1H-indazol-3-yl]- 5- [(methylsulfonyl) methyl]-N- (pyridin-4- yl)pyrimidin-4- amine | ¹H-NMR (400MHz, DMSO-d₆): δ [ppm] = 1.28 (t, 3H), 3.03 (s, 3H), 4.04 (q, 2H), 4.86 (s, 2H), 5.72 (s, 2H), 6.73-6.85 (m, 2H), 7.23-7.33 (m, 1H), 7.47- 7.56 (m, 1H), 7.88 (d, 1H), 8.00-8.06 (m, 2H), 8.41- 8.49 (m, 3H), 8.59 (s, 1H), 9.14 (s, 1H). |
| 2-3-4 SM = 2-12-17 | 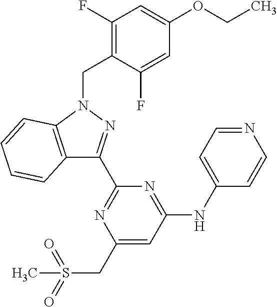 [SMILES: CCOc1cc(F)c(Cn2nc(-c3nc(CS(C)(=O)=O)cc(Nc4ccncc4)n3)c3ccccc32)c(F)c1] | 2-[1-(4-ethoxy- 2,6- difluorobenzyl)- 1H-indazol-3-yl]- 6- [(methylsulfonyl) methyl]-N- (pyridin-4- yl)pyrimidin-4- amine | ¹H-NMR (400MHz, DMSO-d₆): δ [ppm] = 1.30 (t, 3H), 3.17 (s, 3H), 4.05 (q, 2H), 4.76 (s, 2H), 5.73 (s, 2H), 6.82 (d, 2H), 6.96 (s, 1H), 7.31 (t, 1H), 7.53 (t, 1H), 7.90 (d, 1H), 7.94 (d, 2H), 8.44 (d, 2H), 8.60 (d, 1H), 10.28 (s, 1H). |

### Example 2-4-1

### Preparation of 2-{3,5-difluoro-4-[(3-{5-[2-(methylsulfonyl)ethyl]-4-(eparate-4-ylamino)pyrimidin-2-yl}-1H-indazol-1-yl)methyl]phenoxy}ethanol

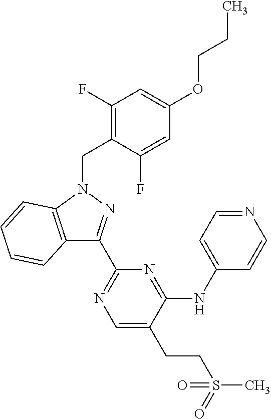 [SMILES: CCCOc1cc(F)c(Cn2nc(-c3ncc(CCS(C)(=O)=O)c(Nc4ccncc4)n3)c3ccccc32)c(F)c1]

51 mg of 2-{1-[4-(2-{[tert-butyl(dimethyl)silyl]oxy}ethoxy)-2,6-difluorobenzyl]-1H-indazol-3-yl}-5-[2-(methylsulfonyl)ethyl]-N-(pyridin-4-yl)pyrimidin-4-amine 2-5-1 (96%, 0.071, 1.0 eq.) were dissolved in dioxane. Then 0.071 mL of hydrochloric acid in dioxane (4 M, 0.28 mmol, 4.0 eq.) was added and the mixture was stirred for 15 min. at room temperature. The reaction mixture was diluted with EE and saturated sodiumhydrogencarbonate-solution and stirred for 30 min. A white precipitation was filtered off under vacuo and dried over night at 50° C. in a vacuum drying oven (fc). The layers of the filtrate were eparatel and the aqueous layer extracted with EE twice. The combined organic layers were dried in use of a waterresistant filter and the filtrate was concentrated under reduced pressure (crude product). The filter cake and the crude product were separately purified by HPLC (basic). The product fractions were combined and lyophilized: 8.9 mg, 0.02 mmol, 21% of the 98% pure target compound.

¹H-NMR (400 MHz, DMSO-d₆): δ [ppm]=3.07 (s, 3H), 3.15-3.23 (m, 2H), 3.55 (t, 2H), 3.66 (br. S., 2H), 4.00 (t, 2H), 4.81-4.94 (m, 1H), 5.70 (s, 2H), 6.73-6.86 (m, 2H), 7.20-7.31 (m, 1H), 7.44-7.54 (m, 1H), 7.85 (d, 1H), 7.96-8.10 (m, 2H), 8.38-8.55 (m, 4H), 9.03 (s, 1H).

The following compounds were prepared according to the same procedure from the indicated starting materials (SM=starting material):

| | | | |
|---|---|---|---|
| 2-4-2 SM = 2-5-2 | 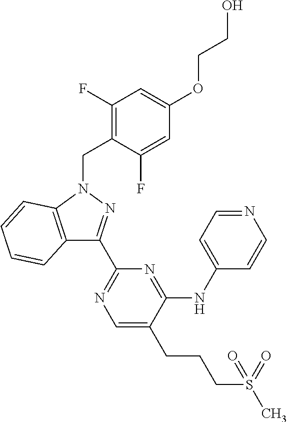 [SMILES: CS(=O)(=O)CCCc1cnc(-c2nn(Cc3c(F)cc(OCCO)cc3F)c3ccccc23)nc1Nc1ccncc1] | 2-{3,5-difluoro-4- [(3-{5-[3- (methylsulfonyl) propyl]-4- (pyridin-4- ylamino) pyrimidin- 2-yl}-1H- indazol-1- yl)methyl] phenoxy} ethanol | ¹H-NMR (400MHz, DMSO-d₆): δ [ppm] = 1.96-2.10 (m, 2H), 2.88 (t, 2H), 2.97 (s, 3H), 3.13- 3.23 (m, 2H), 3.62-3.72 (m, 2H), 4.00 (t, 2H), 4.87 (t, 1H), 5.70 (s, 2H), 6.73-6.86 (m, 2H), 7.20-7.31 (m, 1H), 7.45-7.55 (m, 1H), 7.87 (d, 1H), 8.03-8.12 (m, 2H), 8.36- 8.50 (m, 4H), 8.88 (s, 1H). |
| 2-4-3 SM = 2-5-3 | 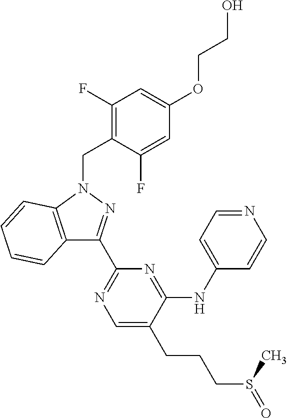 [SMILES: C[S@@](=O)CCCc1cnc(-c2nn(Cc3c(F)cc(OCCO)cc3F)c3ccccc23)nc1Nc1ccncc1] | 2-[3,5-difluoro-4- ({3-[5-{3-[(R)- methylsulfinyl] propyl}-4-(pyridin- 4-ylamino) pyrimidin- 2-yl]-1H- indazol-1- yl}methyl) phenoxy]ethanol | ¹H-NMR (400MHz, DMSO-d₆): δ [ppm] = 1.87-2.02 (m, 2H), 2.53 (s, 3H), 2.67-2.79 (m, 4H), 3.65-3.73 (m, 2H), 3.94- 4.04 (m, 2H), 5.47-5.57 (m, 2H), 6.17-6.31 (m, 2H), 7.13-7.23 (m, 3H), 7.37- 7.45 (m, 1H), 7.66 (d, 2H), 7.73 (d, 1H), 8.26 (s, 1H), 8.46 (d, 1H). |
| 2-4-4 SM: 2-2-3 Side product of 2-3-3 | 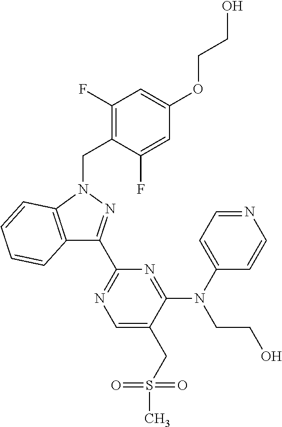 [SMILES: CS(=O)(=O)Cc1cnc(-c2nn(Cc3c(F)cc(OCCO)cc3F)c3ccccc23)nc1N(CCO)c1ccncc1] | 2-[(2-{1-[2,6- difluoro-4-(2- hydroxyethoxy) benzyl]-1H- indazol-3-yl}-5- [(methylsulfonyl) methyl]pyrimidin- 4-yl)(pyridin-4- yl)amino]ethanol | ¹H-NMR (300MHz, DMSO-d₆): δ [ppm] = 2.90 (s, 3H), 3.60- 3.69 (m, 2H), 3.69-3.77 (m, 2H), 3.93-4.02 (m, 2H), 4.03- 4.13 (m, 2H), 4.50 (s, 2H), 4.91 (br. s, 1H), 5.18 (br. s, 1H), 5.67 (s, 2H), 6.72-6.86 (m, 2H), 7.16-7.27 (m, 1H), 7.35-7.53 (m, 3H), 7.71- 7.87 (m, 3H), 8.40 (s, 1H), 8.48 (d, 1H). |
| 2-4-5 SM: 2-5-4 Reac- tion time over night | 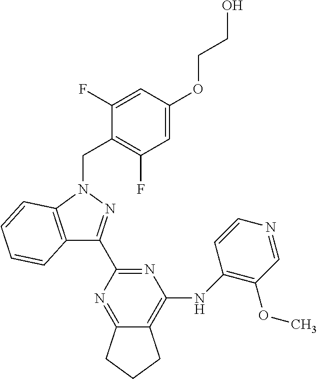 [SMILES: COc1cnccc1Nc1nc(-c2nn(Cc3c(F)cc(OCCO)cc3F)c3ccccc23)nc2c1CCC2] | 2-{3,5-difluoro-4- [(3-{4-[(3- methoxypyridin- 4-yl)amino]-6,7- dihydro-5H- cyclopenta[d] pyrimidin-2-yl}- 1H-indazol-1- yl)methyl] phenoxy}ethanol | ¹H-NMR (400MHz, DMSO-d₆): δ [ppm] = 2.01-2.19 (m, 2 H) 2.84-3.08 (m, 4 H) 3.63- 3.76 (m, 2 H) 3.91-4.08 (m, 5 H) 4.90 (br. s., 1 H) 5.70 (s, 2 H) 6.80 (s, 1 H) 6.83 (s, 1 H) 7.23 (t, 1 H) 7.41-7.56 (m, 1 H) 7.84 (d, 1 H) 7.92 (br. s., 1 H) 8.17 (d, 1 H) 8.34 (s, 1 H) 8.42 (d, 1 H) 8.61 (d, 1 H). |
| 2-4-6 SM: 2-5-5 Reac- tion time over night | 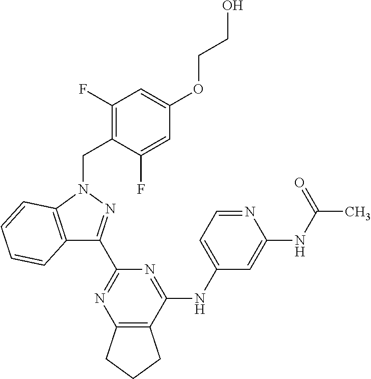 [SMILES: CC(=O)Nc1cc(Nc2nc(-c3nn(Cc4c(F)cc(OCCO)cc4F)c4ccccc34)nc3c2CCC3)ccn1] | N-{4-[(2-{1-[2,6- difluoro-4-(2- hydroxyethoxy) benzyl]-1H- indazol-3-yl}-6,7- dihydro-5H- cyclopenta[d] pyrimidin-4- yl)amino]pyridin- 2-yl}acetamide | ¹H-NMR (400MHz, DMSO-d₆): δ [ppm] = 2.06-2.20 (m, 5 H) 2.87-3.09 (m, 4 H) 3.68 (d, 2 H) 4.01 (t, 2 H) 4.88 (br. s., 1 H) 5.71 (s, 2 H) 6.80 (s, 1 H) 6.82 (s, 1 H) 7.24 (t, 1 H) 7.40- 7.55 (m, 1 H) 7.83 (d, 1 H) 8.14 (d, 1 H) 8.20 (s, 1 H) 8.36 (dd, 1 H) 8.50 (d, 1 H) 9.38 (s, 1 H) 10.32 (s, 1 H). |
| 2-4-7 SM: 2-5-5 Side product of 2-4-6 | 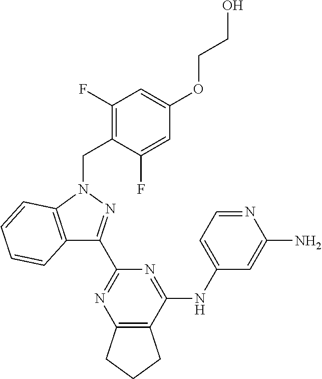 [SMILES: Nc1cc(Nc2nc(-c3nn(Cc4c(F)cc(OCCO)cc4F)c4ccccc34)nc3c2CCC3)ccn1] | 2-{4-[(3-{4-[(2- aminopyridin-4- yl)amino]-6,7- dihydro-5H- cyclopenta[d] pyrimidin-2-yl}- 1H-indazol-1- yl)methyl]-3,5- difluorophenoxy} ethanol | ¹H-NMR (400MHz, DMSO-d₆): δ [ppm] = 2.10 (quin, 2 H) 2.84- 3.08 (m, 4 H) 3.68 (t, 2 H) 3.97-4.09 (m, 2 H) 4.91 (br. s., 2 H) 5.61-5.75 (m, 4 H) 6.79 (s, 1 H) 6.81 (s, 1 H) 7.24 (t, 1 H) 7.34 (dd, 1 H) 7.48 (td, 1 H) 7.73-7.87 (m, 2 H) 8.54 (d, 1 H) 8.78-9.01 (m, 1 H). |
| 2-4-8 SM: 2-5-6 Reac- tion time over night | 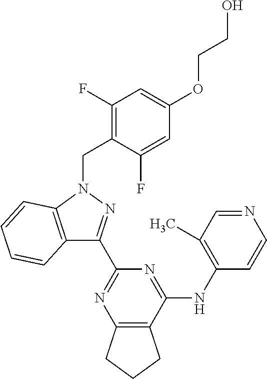 [SMILES: Cc1cnccc1Nc1nc(-c2nn(Cc3c(F)cc(OCCO)cc3F)c3ccccc23)nc2c1CCC2] | 2-{3,5-difluoro-4- [(3-{4-[(3- methylpyridin-4- yl)amino]-6,7- dihydro-5H- cyclopenta[d] pyrimidin-2-yl}- 1H-indazol-1- yl)methyl] phenoxy}ethanol | ¹H-NMR (400MHz, DMSO-d₆): δ [ppm] = 2.12 (quin, 2 H) 2.25 (s, 3 H) 2.82-3.03 (m, 4 H) 3.67-3.69 (m, 2 H) 4.00- 4.03 (m, 2 H) 4.91 (b. s., 1 H) 5.66 (s, 2 H) 6.78 (s, 1 H) 6.81 (s, 1 H) 7.10 (t, 1 H) 7.43 (ddd, 1 H) 7.74-7.85 (m, 2 H) 8.09-8.28 (m, 1 H) 8.37 (d, 1 H) 8.39-8.47 (m, 2 H). |
| 2-4-9 SM: 2-15-8 | 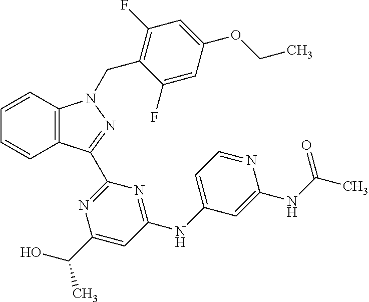 [SMILES: CCOc1cc(F)c(Cn2nc(-c3nc(Nc4ccnc(NC(C)=O)c4)cc([C@H](C)O)n3)c3ccccc32)c(F)c1] | N-[4-({2-[1-(4- ethoxy-2,6- difluorobenzyl)- 1H-indazol-3-yl]- 6-[(1S)-1- hydroxyethyl] pyrimidin-4- yl}amino)pyridin- 2-yl]acetamide | ¹H-NMR (400MHz, DMSO-d₆): δ [ppm] = 1.28 (t, 3H), 1.46 (d, 3H), 2.09 (s, 3H), 4.03 (q, 2H), 4.64-4.75 (m, 1H), 5.52 (d, 1H), 5.70 (s, 2H), 6.72-6.83 (m, 2H), 7.03 (s, 1H), 7.28 (t, 1H), 7.45-7.53 (m, 1H), 7.83 (d, 1H), 7.96-8.02 (m, 1H), 8.10-8.16 (m, 1H), 8.40- 8.47 (m, 1H), 8.51 (d, 1H), 10.17 (s, 1H), 10.32 (s, 1H). |
| 2-4-10 SM: 2-5-7 | 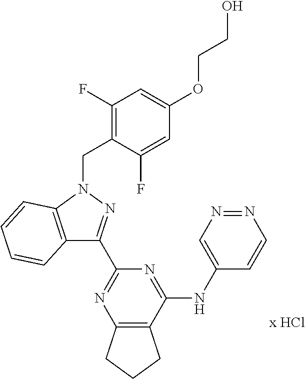 [SMILES: OCCOc1cc(F)c(Cn2nc(-c3nc4c(c(Nc5ccnnc5)n3)CCC4)c3ccccc32)c(F)c1] | 2-[3,5-difluoro-4- ({3-[4-(pyridazin- 4-ylamino)-6,7- dihydro-5H- cyclopenta[d] pyrimidin-2-yl]- 1H-indazol-1- yl}methyl) phenoxy]ethanol, salt with hydrochloric acid | ¹H-NMR (400MHz, DMSO-d₆): δ [ppm] = 1.96-2.23 (m, 2 H) 2.88-3.11 (m, 4 H) 3.68 (t, 2 H) 4.02 (t, 2 H) 4.91 (br. s., 1 H) 5.53-5.79 (m, 2 H) 6.83 (s, 1 H) 6.85 (s, 1 H) 7.18-7.34 (m, 1 H) 7.41-7.57 (m, 1 H) 7.86 (d, 1 H) 8.39-8.57 (m, 1 H) 8.72 (dd, 1 H) 8.95 (d, 1 H) 9.56 (d, 2 H). |
| 2-4-11 SM: 2-5-8 | 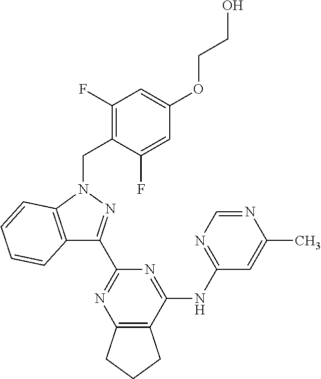 [SMILES: Cc1cc(Nc2nc(-c3nn(Cc4c(F)cc(OCCO)cc4F)c4ccccc34)nc3c2CCC3)ncn1] | 2-{3,5-difluoro-4- [(3-{4-[(6- methylpyrimidin- 4-yl)amino]-6,7- dihydro-5H- cyclopenta[d] pyrimidin-2-yl}- 1H-indazol-1- yl)methyl] phenoxy}ethanol | ¹H-NMR (400MHz, DMSO-d₆): δ [ppm] = 2.02-2.21 (m, 2 H) 2.55 (s, 3 H) 2.97-3.11 (m, 2 H) 3.42-3.49 (m, 2 H) 3.62- 3.69 (m, 2 H) 3.78-3.87 (m, 1 H) 3.93-4.04 (m, 2 H) 5.74 (s, 2 H) 6.77 (s, 1 H) 6.79 (s, 1 H) 7.21-7.37 (m, 1 H) 7.44- 7.57 (m, 1 H) 7.85 (d, 1 H) 8.45-8.56 (m, 2 H) 8.64 (br. s., 1 H) 9.03 (s, 1 H) 11.12 (br. s., 1 H). |
| 2-4-12 SM: 2-5-9 | 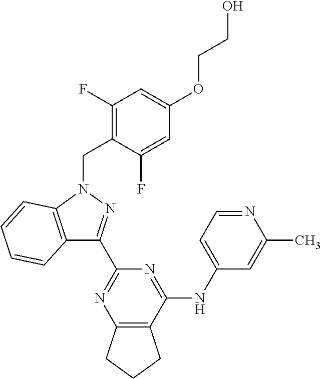 [SMILES: Cc1cc(Nc2nc(-c3nn(Cc4c(F)cc(OCCO)cc4F)c4ccccc34)nc3c2CCC3)ccn1] | 2-{3,5-difluoro-4- [(3-{4-[(2- methylpyridin-4- yl)amino]-6,7- dihydro-5H- cyclopenta[d] pyrimidin-2-yl}- 1H-indazol-1- yl)methyl] phenoxy}ethanol | ¹H-NMR (500MHz, DMSO-d₆): δ [ppm] = 2.14 (quin, 2 H) 2.60 (s, 3 H) 3.02-3.05 (m, 4 H) 3.66 (t, 2 H) 3.95-4.09 (m, 2 H) 4.87 (br. s., 1 H) 5.73 (s, 2 H) 6.78 (s, 1 H) 6.80 (s, 1 H) 7.22-7.38 (m, 1 H) 7.51 (dd, 1 H) 7.85 (d, 1 H) 8.14 (br. s., 1 H) 8.41 (d, 1 H) 8.50 (d, 1 H) 8.55 (br. s., 1 H) 10.18 (br. s., 1 H). |

### Example 2-5-1

### Preparation of 2-{1-[4-(2-{[tert-butyl(dimethyl)silyl]oxy}ethoxy)-2,6-difluorobenzyl]-1H-indazol-3-yl}-5-[2-(methylsulfonyl)ethyl]-N-(pyridin-4-yl)pyrimidin-4-amine

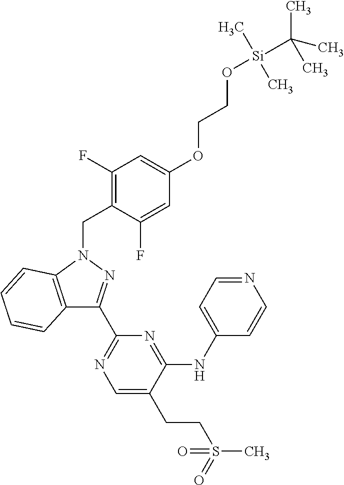 [SMILES: CC(C)(C)[Si](C)(C)OCCOc1cc(F)c(Cn2nc(-c3ncc(CCS(C)(=O)=O)c(Nc4ccncc4)n3)c3ccccc32)c(F)c1]

71 mg of 3,5-difluoro-4-[(3-{5-[2-(methylsulfonyl)ethyl]-4-(pyridin-4-ylamino)pyrimidin-2-yl}-1H-indazol-1-yl)methyl]phenol 2-6-1 (0.13 mmol, 1.00 eq.) was dissolved in 3 mL DMF. Then 91 mg potassium carbonate (0.66 mmol, 5.0 eq.) and 48 mg (2-bromoethoxy)(tert-butyl)dimethylsilane (0.20 mmol, 1.50 eq.) were added. The mixture was stirred over night at 60° C. It was extracted with ethyl acetate twice, dried over a waterresistend filter and concentrated under reduced pressure. The crude product was purified by flash chromatography to provide 51 mg of 96% pure target compound (0.07 mmol, 53%).

LC-MS (Methode with NH₃):

retention time: 1.55 min

MS ES+: 695.3 [M+H]⁺

The following compounds were prepared according to the same procedure from the indicated starting materials (SM=starting material):

| | | | |
|---|---|---|---|
| 2-5-2 SM = 2-6-2 | 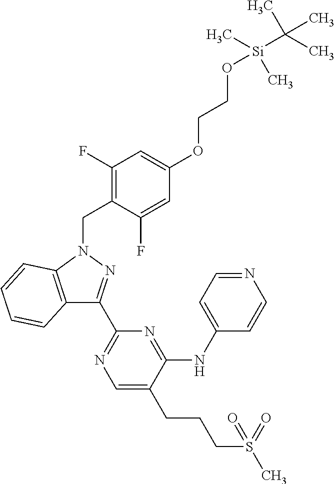 [SMILES: CC(C)(C)[Si](C)(C)OCCOc1cc(F)c(Cn2nc(-c3ncc(CCCS(C)(=O)=O)c(Nc4ccncc4)n3)c3ccccc32)c(F)c1] | 2-{1-[4-(2-{[tert- butyl(dimethyl) silyl]oxy}ethoxy)- 2,6- difluorobenzyl]- 1H-indazol-3-yl}- 5-[3- (methylsulfonyl) propyl]-N-(pyridin- 4-yl)pyrimidin-4- amine | LC-MS (Methode with NH₃): retention time: 1.55 min MS ES+: 709.3 [M + H]⁺ |
| 2-5-3 SM = 2-6-3 | 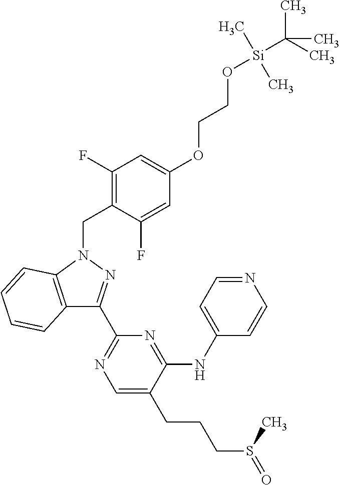 [SMILES: C[S@@](=O)CCCc1cnc(-c2nn(Cc3c(F)cc(OCCO[Si](C)(C)C(C)(C)C)cc3F)c3ccccc23)nc1Nc1ccncc1] | 2-{1-[4-(2-{[tert- butyl(dimethyl) silyl]oxy}ethoxy)- 2,6- difluorobenzyl]- 1H-indazol-3-yl}- 5-{3-[(R)- methylsulfinyl] propyl}-N-(pyridin- 4-yl)pyrimidin-4- amine | LC-MS (Methode with NH₃): retention time: 1.33 min MS ES+: 693.4 [M + H]⁺ |
| 2-5-4 SM = 2-6-6 | 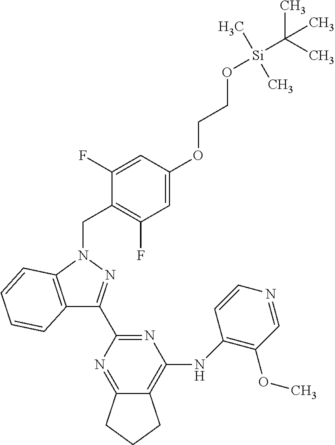 [SMILES: COc1cnccc1Nc1nc(-c2nn(Cc3c(F)cc(OCCO[Si](C)(C)C(C)(C)C)cc3F)c3ccccc23)nc2c1CCC2] | 2-{1-[4-(2-{[tert- butyl(dimethyl) silyl]oxy}ethoxy)- 2,6- difluorobenzyl]- 1H-indazol-3-yl}- N-(3- methoxypyridin- 4-yl)-6,7-dihydro- 5H- cyclopenta[d] pyrimidin-4-amine | LC-MS (Methode with form acid): retention time: 1.48 min MS ES+: 660.6 [M + H]⁺ |
| 2-5-5 SM = 2-6-7 | 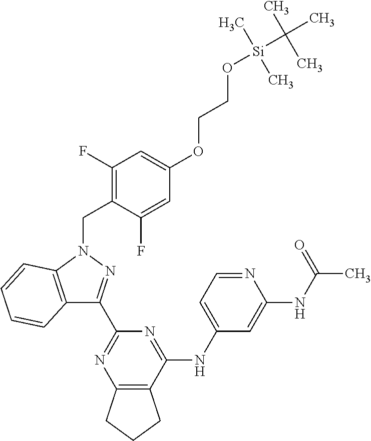 [SMILES: CC(=O)Nc1cc(Nc2nc(-c3nn(Cc4c(F)cc(OCCO[Si](C)(C)C(C)(C)C)cc4F)c4ccccc34)nc3c2CCC3)ccn1] | N-{4-[(2-{1-[4-(2- {[tert- butyl(dimethyl) silyl]oxy}ethoxy)- 2,6- difluorobenzyl]- 1H-indazol-3-yl}- 6,7-dihydro-5H- cyclopenta[d] pyrimidin-4- yl)amino]pyridin- 2-yl}acetamide | LC-MS (Methode with formic acid): retention time: 1.68 min MS ES+: 688.5 [M + H]⁺ |
| 2-5-6 SM = 2-6-5 | 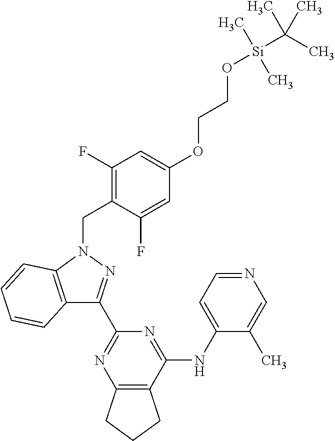 [SMILES: Cc1cnccc1Nc1nc(-c2nn(Cc3c(F)cc(OCCO[Si](C)(C)C(C)(C)C)cc3F)c3ccccc23)nc2c1CCC2] | 2-{1-[4-(2-{[tert- butyl(dimethyl) silyl]oxy}ethoxy)- 2,6- difluorobenzyl]- 1H-indazol-3-yl}- N-(3- methylpyridin-4- yl)-6,7-dihydro- 5H- cyclopenta[d] pyrimidin-4-amine | LC-MS (Methode with formic acid): retention time: 1.72 min MS ES+: 643.5 [M + H]⁺ |
| 2-5-7 SM = 2-6-9 | 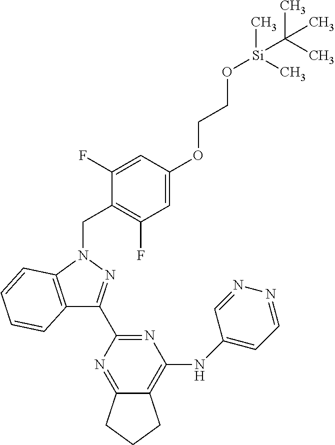 [SMILES: CC(C)(C)[Si](C)(C)OCCOc1cc(F)c(Cn2nc(-c3nc4c(c(Nc5ccnnc5)n3)CCC4)c3ccccc32)c(F)c1] | 2-{1-[4-(2-{[tert- butyl(dimethyl) silyl]oxy}ethoxy)- 2,6- difluorobenzyl]- 1H-indazol-3-yl}- N-(pyridazin-4- yl)-6,7-dihydro- 5H- cyclopenta[d] pyrimidin-4-amine | LC-MS (Methode with NH₃): retention time: 1.68 min MS ES+: 629.0 [M + H]⁺ |
| 2-5-8 SM = 2-6-10 | 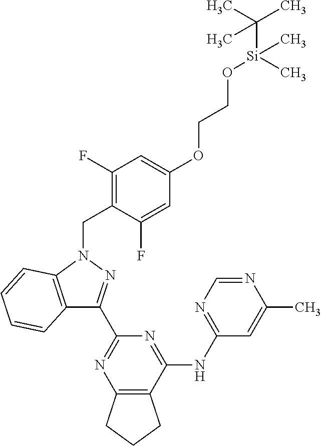 [SMILES: Cc1cc(Nc2nc(-c3nn(Cc4c(F)cc(OCCO[Si](C)(C)C(C)(C)C)cc4F)c4ccccc34)nc3c2CCC3)ncn1] | 2-{1-[4-(2-{[tert- butyl(dimethyl) silyl]oxy}ethoxy)- 2,6- difluorobenzyl]- 1H-indazol-3-yl}- N-(6- methylpyrimidin- 4-yl)-6,7-dihydro- 5H- cyclopenta[d] pyrimidin-4-amine | LC-MS (Methode with NH₃): retention time: 1.72 min MS ES+: 644.0 [M + H]⁺ |
| 2-5-9 SM = 2-6-8 | 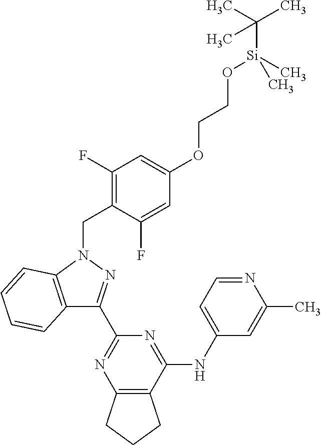 [SMILES: Cc1cc(Nc2nc(-c3nn(Cc4c(F)cc(OCCO[Si](C)(C)C(C)(C)C)cc4F)c4ccccc34)nc3c2CCC3)ccn1] | 2-{1-[4-(2-{[tert- butyl(dimethyl) silyl]oxy}ethoxy)- 2,6- difluorobenzyl]- 1H-indazol-3-yl}- N-(2- methylpyridin-4- yl)-6,7-dihydro- 5H- cyclopenta[d] pyrimidin-4-amine | LC-MS (Methode with NH₃): retention time: 1.44 min MS ES+: 642.8 [M + H]⁺ |

### Example 2-6-1

### Preparation of 3,5-difluoro-4-[(3-{5-[2-(methylsulfonyl)ethyl]-4-(pyridin-4-ylamino)pyrimidin-2-yl}-1H-indazol-1-yl)methyl]phenol

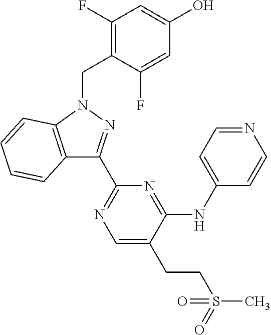 [SMILES: CS(=O)(=O)CCc1cnc(-c2nn(Cc3c(F)cc(O)cc3F)c3ccccc23)nc1Nc1ccncc1]

150 mg of 2-[1-(4-ethoxy-2,6-difluorobenzyl)-1H-indazol-3-yl]-5-[2-(methylsulfonyl)ethyl]-N-(pyridin-4-yl)pyrimidin-4-amine 2-3-1 (0.26 mmol, 1.00 eq.) was dissolved in 6.8 mL dichloromethane. 1.06 mL Boron tribromide-solution (1 M, in dichloromethane, 1.06 mmol, 4.0 eq.) were added dropwise. It was stirred at rt for 30 minutes. The reaction mixture was quenched with ice water and diluted with dichloromethane. Using a 2 M aqueous sodium hydroxide solution the pH value was turned from 1 to 10. The layers were separated. The aqueous layer was extracted with DCM twice. The combined organic layers were washed with brine and they were dried using a waterresistant filter, concentrated in vacuo, but contained no product.

The aqueous layer was concentrated under vacuo and extracted with ethyl acetate three times. The combined organic layers were concentrated under reduced pressure under vacuo The second crude product was suspended in dichloromethan/methanol 1:1. A white solid was filtered off to provide 334 mg of the target compound in 93% purity, 234%.

LC-MS (Methode with NH₃):

retention time: 0.72 min

MS ES+: 537.2 [M+H]⁺

The following compounds were prepared according to the same procedure from the indicated starting materials (SM=starting material):

| | | | |
|---|---|---|---|
| 2-6-2 SM = 2-3-2 | 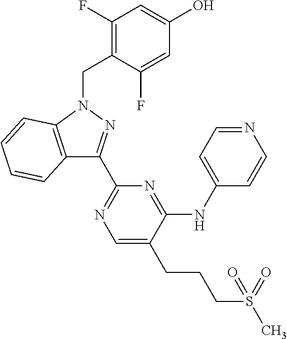 [SMILES: CS(=O)(=O)CCCc1cnc(-c2nn(Cc3c(F)cc(O)cc3F)c3ccccc23)nc1Nc1ccncc1] | 3,5-difluoro-4-[(3- {5-[3- (methylsulfonyl) propyl]-4- (pyridin-4- ylamino) pyrimidin- 2-yl}-1H- indazol-1- yl)methyl]phenol | LC-MS (Methode with NH₃): retention time: 0.76 min MS ES+: 551.2 [M + H]⁺ |
| 2-6-3 SM = 2-1-2 | 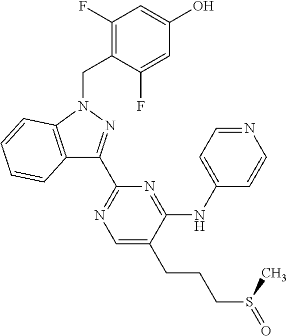 [SMILES: C[S@@](=O)CCCc1cnc(-c2nn(Cc3c(F)cc(O)cc3F)c3ccccc23)nc1Nc1ccncc1] | 3,5-difluoro-4- ({3-[5-{3-[(R)- methylsulfinyl] propyl}-4- (pyridin-4- ylamino) pyrimidin- 2-yl]-1H- indazol-1- yl}methyl)phenol | LC-MS (Methode with NH₃): retention time: 0.72 min MS ES+: 535.2 [M + H]⁺ |
| 2-6-4 SM = 2-12-19 | 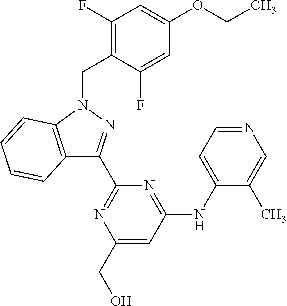 [SMILES: CCOc1cc(F)c(Cn2nc(-c3nc(CO)cc(Nc4ccncc4C)n3)c3ccccc32)c(F)c1] | {2-[1-(4-ethoxy- 2,6- difluorobenzyl)- 1H-indazol-3-yl]- 6-[(3- methylpyridin-4- yl)amino] pyrimidin- 4-yl}methanol | ¹H-NMR (400MHz, DMSO-d₆): δ [ppm] = 1.28 (t, 3H), 2.30 (s, 3H), 4.03 (q, 2H), 4.57 (d, 2H), 5.58 (t, 1H), 5.68 (s, 2H), 6.71- 6.81 (m, 2H), 7.14-7.25 (m, 2H), 7.46 (t, 1H), 7.80 (d, 1H), 8.26-8.33 (m, 2H), 8.35 (s, 1H), 8.42 (d, 1H), 9.06 (s, 1H). |
| 2-6-5 SM = 2-14-2 3 d at 50° C. reaction | 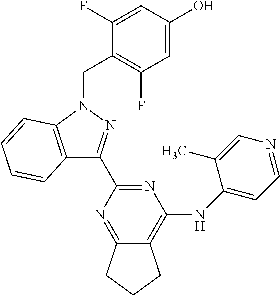 [SMILES: Cc1cnccc1Nc1nc(-c2nn(Cc3c(F)cc(O)cc3F)c3ccccc23)nc2c1CCC2] | 3,5-difluoro-4-[(3- {4-[(3- methylpyridin-4- yl)amino]-6,7- dihydro-5H- cyclopenta[d] pyrimidin-2-yl}-1H- indazol-1- yl)methyl]phenol | ¹H-NMR (400MHz, DMSO-d₆): δ [ppm] = 2.10 (quin, 2 H) 2.24 (s, 3 H) 2.83-3.06 (m, 4 H) 5.60 (s, 2 H) 6.47 (s, 1 H) 6.50 (s, 1 H) 7.08 (t, 1 H) 7.41 (ddd, 1 H) 7.74 (d, 1 H) 7.80 (d, 1 H) 8.00-8.23 (m, 1 H) 8.29-8.51 (m, 3 H) 10.49 (br. s., 1 H). |
| 2-6-6 SM = 2-14-4 7 d at 50° C. reaction | 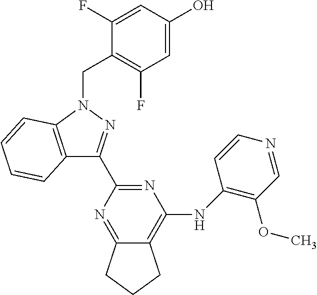 [SMILES: COc1cnccc1Nc1nc(-c2nn(Cc3c(F)cc(O)cc3F)c3ccccc23)nc2c1CCC2] | 3,5-difluoro-4-[(3- {4-[(3- methoxypyridin- 4-yl)amino]-6,7- dihydro-5H- cyclopenta[d] pyrimidin-2-yl}-1H- indazol-1- yl)methyl]phenol | LC-MS (Methode with formic acid): retention time: 1.05 min MS ES−: 499.2 [M − H]⁺ |
| 2-6-7 SM = 2-12-18 3 d at 50° C. reaction | 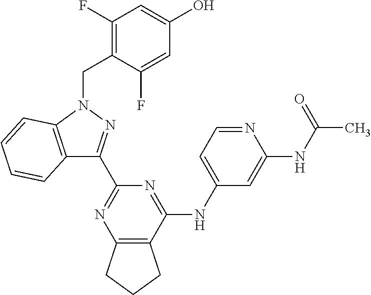 [SMILES: CC(=O)Nc1cc(Nc2nc(-c3nn(Cc4c(F)cc(O)cc4F)c4ccccc34)nc3c2CCC3)ccn1] | N-[4-({2-[1-(2,6- difluoro-4- hydroxybenzyl)- 1H-indazol-3-yl]- 6,7-dihydro-5H- cyclopenta[d] pyrimidin-4- yl}amino)pyridin- 2-yl]acetamide | ¹H-NMR (400MHz, DMSO-d₆): δ [ppm] = 1.94-2.18 (m, 5 H) 2.80-3.09 (m, 4 H) 5.64 (s, 2 H) 6.49 (s, 1 H) 6.51 (s, 1 H) 7.22 (t, 1 H) 7.36-7.61 (m, 1 H) 7.80 (d, 1 H) 8.11 (d, 1 H) 8.18 (s, 1 H) 8.36 (dd, 1 H) 8.48 (d, 1 H) 9.36 (s, 1 H) 10.29 (s, 1 H) 10.49 (br. s., 1 H). |
| 2-6-8 SM = 2-12-6 7 d at 50° C. reaction | 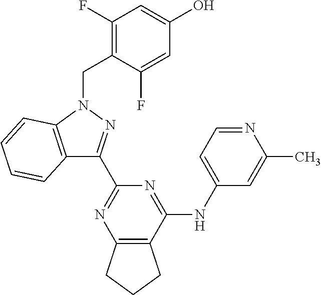 [SMILES: Cc1cc(Nc2nc(-c3nn(Cc4c(F)cc(O)cc4F)c4ccccc34)nc3c2CCC3)ccn1] | 3,5-difluoro-4-[(3- {4-[(2- methylpyridin-4- yl)amino]-6,7- dihydro-5H- cyclopenta[d] pyrimidin-2-yl}-1H- indazol-1- yl)methyl]phenol | ¹H-NMR (400MHz, DMSO-d₆): δ [ppm] = 2.13 (quin, 2 H) 2.44 (s, 3 H) 2.89-3.06 (m, 4 H) 5.67 (s, 2 H) 6.50 (s, 1 H) 6.52 (s, 1 H) 7.26 (t, 1 H) 7.49 (td, 1 H) 7.73-7.90 (m, 2 H) 7.97 (d, 1 H) 8.28 (d, 1 H) 8.52 (d, 1 H) 9.08 (s, 1 H) 10.52 (br. s., 1 H). |
| 2-6-9 SM = 2-14-5 5 d at 50° C. reaction | 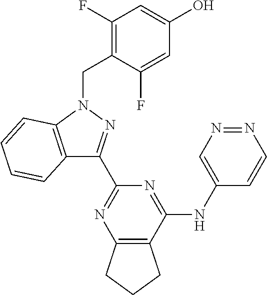 [SMILES: Oc1cc(F)c(Cn2nc(-c3nc4c(c(Nc5ccnnc5)n3)CCC4)c3ccccc32)c(F)c1] | 3,5-difluoro-4- ({3-[4-(pyridazin- 4-ylamino)-6,7- dihydro-5H- cyclopenta[d] pyrimidin-2-yl]-1H- indazol-1- yl}methyl)phenol | LC-MS (Methode with NH₃): retention time: 0.79 min MS ES+: 472.2 [M + H]⁺ |
| 2-6-10 SM = 2-21-1 7 d at 50° C. reaction | 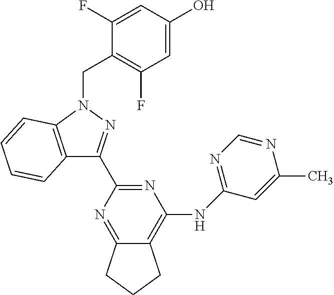 [SMILES: Cc1cc(Nc2nc(-c3nn(Cc4c(F)cc(O)cc4F)c4ccccc34)nc3c2CCC3)ncn1] | 3,5-difluoro-4-[(3- {4-[(6- methylpyrimidin- 4-yl)amino]-6,7- dihydro-5H- cyclopenta[d] pyrimidin-2-yl}-1H- indazol-1- yl)methyl]phenol | LC-MS (Methode with NH₃): retention time: 0.81 min MS ES+: 486.3 [M + H]⁺ |
| 2-6-11 SM = 2-14-6 3 d at 50° C. reaction | 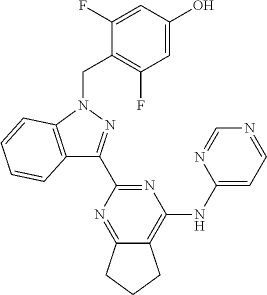 [SMILES: Oc1cc(F)c(Cn2nc(-c3nc4c(c(Nc5ccncn5)n3)CCC4)c3ccccc32)c(F)c1] | 3,5-difluoro-4- ({3-[4-(pyrimidin- 4-ylamino)-6,7- dihydro-5H- cyclopenta[d] pyrimidin-2-yl]-1H- indazol-1- yl}methyl)phenol | ¹H-NMR (400MHz, DMSO-d₆): δ [ppm] = 2.04-2.21 (m, 2 H) 2.98-3.04 (m, 4 H) 5.46 (s, 2 H) 5.53-5.73 (m, 2 H) 7.24 (t, 1 H) 7.32-7.51 (m, 1 H) 7.74 (d, 1 H) 8.40-8.60 (m, 2 H) 8.65 (br. s., 1 H) 8.84 (s, 1 H) 10.05 (br. s., 1 H). |
| 2-6-12 SM = 2-14-54 7 d at 50° C. reaction | 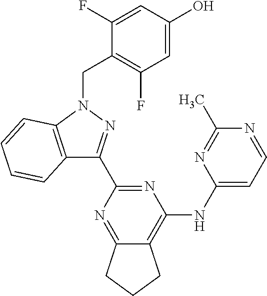 [SMILES: Cc1nccc(Nc2nc(-c3nn(Cc4c(F)cc(O)cc4F)c4ccccc34)nc3c2CCC3)n1] | 3,5-difluoro-4-[(3- {4-[(2- methylpyrimidin- 4-yl)amino]-6,7- dihydro-5H- cyclopenta[d] pyrimidin-2-yl}-1H- indazol-1- yl)methyl]phenol | LC-MS (Methode with formic acid): retention time: 1.07 min MS ES−: 486.2 [M − H]⁺ |
| 2-6-13 SM = 2-20-7 1 d at rt reaction | 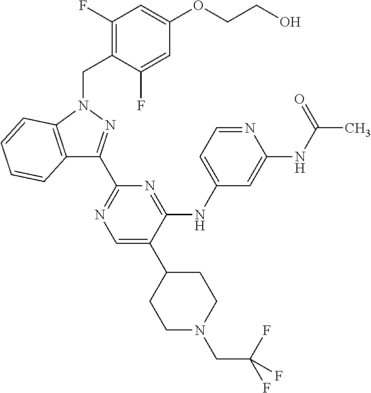 [SMILES: CC(=O)Nc1cc(Nc2nc(-c3nn(Cc4c(F)cc(OCCO)cc4F)c4ccccc34)ncc2C2CCN(CC(F)(F)F)CC2)ccn1] | N-{4-[(2-{1-[2,6- difluoro-4-(2- hydroxyethoxy) benzyl]-1H- indazol-3-yl}-5- [1-(2,2,2- trifluoroethyl) piperidin-4- yl]pyrimidin-4- yl)amino]pyridin- 2-yl}acetamide | ¹H-NMR (400 MHz, DMSO- d₆): δ [ppm] = 1.73 (m, 2H), 1.81 (br. d., 2H), 2.08 (s, 3H), 2.61 (m, 2H), 3.04 (m, 2H), 3.11 (m, 1H), 3.26 (q, 2H), 3.67 (td, 2H), 4.00 (t, 2H), 4.89 (t, 1H), 5.69 (s, 2H), 6.79 (m, 2H), 7.19 (dd, 1H), 7.47 (dd, 1H), 7.83 (d, 1H), 8.12 (dd, 1H), 8.15 (d, 1H), 8.23 (br. s., 1H), 8.40 (d, 1H), 8.53 (s, 1H), 9.13 (br. s., 1H), 10.37 (br. s., 1H). |
| 2-6-14 SM = 2-20-8 3 h at rt reaction | 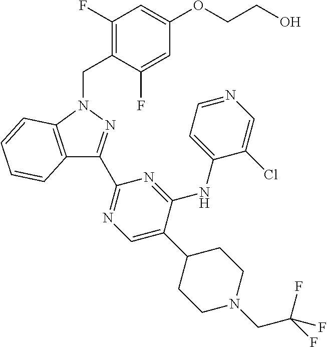 [SMILES: OCCOc1cc(F)c(Cn2nc(-c3ncc(C4CCN(CC(F)(F)F)CC4)c(Nc4ccncc4Cl)n3)c3ccccc32)c(F)c1] | 2-{4-[(3-{4-[(3- chloropyridin-4- yl)amino]-5-[1- (2,2,2- trifluoroethyl) piperidin-4- yl]pyrimidin-2-yl}- 1H-indazol-1- yl)methyl]-3,5- difluorophenoxy} ethanol | ¹H-NMR (400 MHz, DMSO- d₆): δ [ppm] = 1.78 (m, 2H), 1.92 (br. d., 2H), 2.57 (m, 2H), 2.89 (m, 1H), 3.08 (m, 2H), 3.25 (q, 2H), 3.67 (td, 2H), 4.00 (t, 2H), 4.89 (t, 1H), 5.67 (s, 2H), 6.80 (m, 2H), 7.10 (dd, 1H), 7.44 (dd, 1H), 7.81 (d, 1H), 8.03 (d, 1H), 8.26 (d, 1H), 8.50 (d, 1H), 8.53 (s, 1H), 8.58 (s, 1H), 8.67 (br. s., 1H). |
| 2-6-15 SM = 2-20-9 19 h at rt reaction | 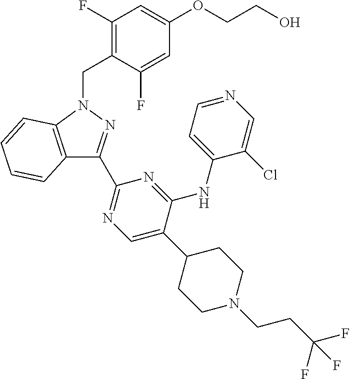 [SMILES: OCCOc1cc(F)c(Cn2nc(-c3ncc(C4CCN(CCC(F)(F)F)CC4)c(Nc4ccncc4Cl)n3)c3ccccc32)c(F)c1] | 2-{4-[(3-{4-[(3- chloropyridin-4- yl)amino]-5-[1- (3,3,3- trifluoropropyl) piperidin-4- yl]pyrimidin-2-yl}- 1H-indazol-1- yl)methyl]-3,5- difluorophenoxy} ethanol | ¹H-NMR (400 MHz, DMSO- d₆): δ [ppm] = 1.74 (m, 2H), 1.94 (br. d., 2H), 2.15 (t, 2H), 2.57 (m, 2H), 2.59 (m, 2H), 2.88 (m, 1H), 3.06 (m, 2H), 3.67 (td, 2H), 4.00 (t, 2H), 4.90 (t, 1H), 5.67 (s, 2H), 6.80 (m, 2H), 7.11 (dd, 1H), 7.44 (dd, 1H), 7.81 (d, 1H), 8.05 (d, 1H), 8.31 (d, 1H), 8.49 (d, 1H), 8.52 (s, 1H), 8.55 (s, 1H), 8.67 (br. s., 1H). |
| 2-6-16 SM = 2-20-10 24 h at rt reaction | 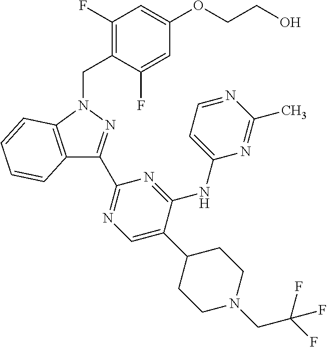 [SMILES: Cc1nccc(Nc2nc(-c3nn(Cc4c(F)cc(OCCO)cc4F)c4ccccc34)ncc2C2CCN(CC(F)(F)F)CC2)n1] | 2-{3,5-difluoro-4- [(3-{4-[(2- methylpyrimidin- 4-yl)amino]-5-[1- (2,2,2- trifluoroethyl) piperidin-4- yl]pyrimidin-2-yl}- 1H-indazol-1- yl)methyl] phenoxy}ethanol | ¹H-NMR (400 MHz, DMSO- d₆): δ [ppm] = 1.74 (m, 2H), 1.77 (br. d., 2H), 2.56 (s, 3H), 2.60 (m, 2H), 3.01 (m, 2H), 3.22 (m, 1H), 3.24 (q, 2H), 3.67 (td, 2H), 4.00 (t, 2H), 4.89 (t, 1H), 5.71 (s, 2H), 6.82 (m, 2H), 7.27 (dd, 1H), 7.50 (dd, 1H), 7.87 (d, 1H), 8.44 (d, 1H), 8.45 (d, 1H), 8.47 (d, 1H), 8.64 (s, 1H), 9.78 (br. s., 1H). |
| 2-6-17 SM = 2-20-11 20 h at rt reaction | 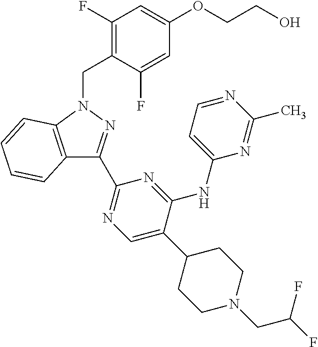 [SMILES: Cc1nccc(Nc2nc(-c3nn(Cc4c(F)cc(OCCO)cc4F)c4ccccc34)ncc2C2CCN(CC(F)F)CC2)n1] | 2-{4-[(3-{5-[1- (2,2- difluoroethyl) piperidin-4-yl]-4- [(2- methylpyrimidin- 4-yl)amino] pyrimidin-2-yl}-1H- indazol-1- yl)methyl]-3,5- difluorophenoxy} ethanol | ¹H-NMR (400 MHz, DMSO- d₆): δ [ppm] = 1.71 (m, 2H), 1.79 (m, 2H), 2.39 (m, 2H), 2.54 (s, 3H), 2.77 (td, 2H), 3.00 (br. d., 2H), 3.18 (m, 1H), 3.67 (td, 2H), 4.00 (t, 2H), 4.90 (t, 1H), 5.70 (s, 2H), 6.18 (tt, 1H), 6.82 (m, 2H), 7.27 (dd, 1H), 7.50 (dd, 1H), 7.87 (d, 1H), 8.41 (d, 1H), 8.46 (d, 1H), 8.46 (d, 1H), 8.62 (s, 1H), 9.77 (br. s., 1H). |
| 2-6-18 SM = 2-12-55 1 h at rt reaction | 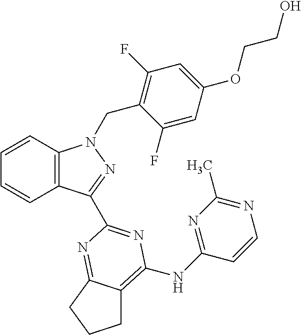 [SMILES: Cc1nccc(Nc2nc(-c3nn(Cc4c(F)cc(OCCO)cc4F)c4ccccc34)nc3c2CCC3)n1] | 2-{3,5-difluoro-4- [(3-{4-[(2- methylpyrimidin- 4-yl)amino]-6,7- dihydro-5H- cyclopenta[d] pyrimidin-2-yl}-1H- indazol-1- yl)methyl] phenoxy}ethanol | ¹H-NMR (400MHz, DMSO-d₆): δ [ppm] = 2.07 (quint, 2H), 2.54 (s, 3H), 2.99 (t, 4H), 3.66 (q, 2H), 4.00 (t, 2H), 4.86 (t, 1H), 5.70 (s, 2H), 6.76-6.86 (m, 2H), 7.27 (t, 1H), 7.45-7.52 (m, 1H), 7.84 (d, 1H), 8.42- 8.48 (m, 2H), 8.50 (d, 1H), 9.91 (s, 1H). |
| 2-6-19 SM = 2-12-56 1 h at rt reaction | 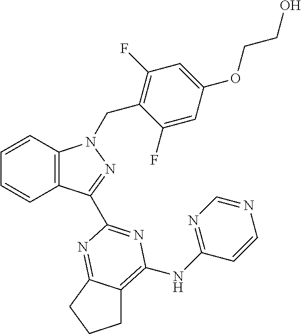 [SMILES: OCCOc1cc(F)c(Cn2nc(-c3nc4c(c(Nc5ccncn5)n3)CCC4)c3ccccc32)c(F)c1] | 2-[3,5-difluoro-4- ({3-[4-(pyrimidin- 4-ylamino)-6,7- dihydro-5H- cyclopenta[d] pyrimidin-2-yl]-1H- indazol-1- yl}methyl) phenoxy]ethanol | ¹H-NMR (400MHz, DMSO-d₆): δ [ppm] = 2.09 (quin, 2H), 2.96- 3.07 (m, 4H), 3.68 (q, 2H), 4.01 (t, 2H), 4.90 (t, 1H), 5.72 (s, 2H), 6.79-6.89 (m, 2H), 7.29 (t, 1H), 7.51 (t, 1H), 7.88 (d, 1H), 8.53 (d, 1H), 8.57 (d, 1H), 8.64-8.69 (m, 1H), 8.84- 8.89 (m, 1H), 10.09 (br. s, 1H). |
| 2-6-20 SM = 2-12-57 1 h at rt reaction | 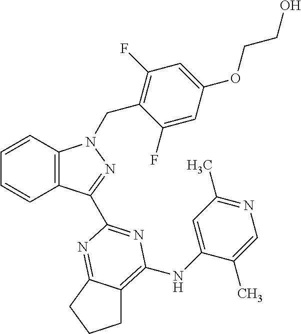 [SMILES: Cc1cc(Nc2nc(-c3nn(Cc4c(F)cc(OCCO)cc4F)c4ccccc34)nc3c2CCC3)c(C)cn1] | 2-{4-[(3-{4-[(2,5- dimethylpyridin- 4-yl)amino]-6,7- dihydro-5H- cyclopenta[d] pyrimidin-2-yl}-1H- indazol-1- yl)methyl]-3,5- difluorophenoxy} ethanol | ¹H-NMR (400MHz, DMSO-d₆): δ [ppm] = 2.12 (quin, 2H), 2.22 (s, 3H), 2.44 (s, 3H), 2.90 (t, 2H), 2.97 (t, 2H), 3.68 (br. t, 2H), 4.01 (t, 2H), 4.93 (br. t, 1H), 5.67 (s, 2H), 6.74-6.84 (m, 2H), 7.11 (t, 1H), 7.44 (t, 1H), 7.70 (s, 1H), 7.77 (d, 1H), 8.23 (d, 1H), 8.30 (d, 2H). |
| 2-6-21 SM = 2-12-58 1 h at rt reaction | 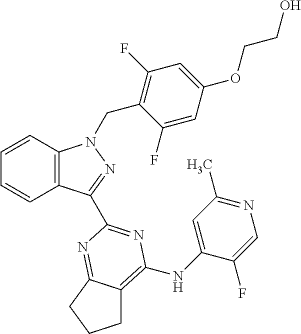 [SMILES: Cc1cc(Nc2nc(-c3nn(Cc4c(F)cc(OCCO)cc4F)c4ccccc34)nc3c2CCC3)c(F)cn1] | 2-{3,5-difluoro-4- [(3-{4-[(5-fluoro- 2-methylpyridin- 4-yl)amino]-6,7- dihydro-5H- cyclopenta[d] pyrimidin-2-yl}-1H- indazol-1- yl)methyl] phenoxy}ethanol | ¹H-NMR (400MHz, DMSO-d₆): δ [ppm] = 2.11 (quin, 2H), 2.46 (s, 3H), 2.97 (q, 4H), 3.68 (q, 2H), 4.01 (t, 2H), 4.90 (t, 1H), 5.70 (s, 2H), 6.74-6.84 (m, 2H), 7.18 (t, 1H), 7.47 (t, 1H), 7.80 (d, 1H), 8.14 (d, 1H), 8.33 (d, 1H), 8.41 (s, 1H), 8.85 (s, 1H). |
| 2-6-22 SM = 2-12-61 1 h at rt reaction | 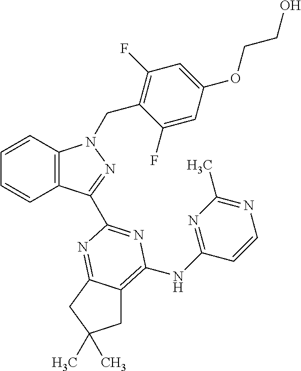 [SMILES: Cc1nccc(Nc2nc(-c3nn(Cc4c(F)cc(OCCO)cc4F)c4ccccc34)nc3c2CC(C)(C)C3)n1] | 2-{4-[(3-{6,6- dimethyl-4-[(2- methylpyrimidin- 4-yl)amino]-6,7- dihydro-5H- cyclopenta[d] pyrimidin-2-yl}-1H- indazol-1- yl)methyl]-3,5- difluorophenoxy} ethanol | ¹H-NMR (400MHz, DMSO-d₆): δ [ppm] = 1.18 (s, 6H), 2.55 (s, 3H), 2.81 (s, 2H), 2.84 (s, 2H), 3.68 (q, 2H), 4.01 (t, 2H), 4.90 (t, 1H), 5.71 (s, 2H), 6.77- 6.88 (m, 2H), 7.24-7.32 (m, 1H), 7.47-7.54 (m, 1H), 7.86 (d, 1H), 8.37 (d, 1H), 8.45 (d, 1H), 8.51 (d, 1H), 9.97 (s, 1H). |
| 2-6-23 SM = 2-20-12 22 h at rt reaction | 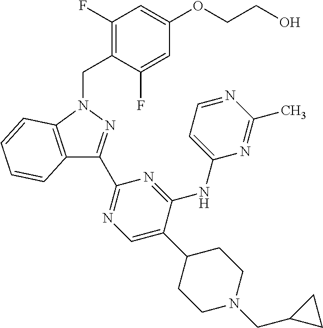 [SMILES: Cc1nccc(Nc2nc(-c3nn(Cc4c(F)cc(OCCO)cc4F)c4ccccc34)ncc2C2CCN(CC3CC3)CC2)n1] | 2-{4-[(3-{5-[1- (cyclopropyl- methyl)piperidin- 4-yl]-4-[(2- methylpyrimidin- 4- yl)amino] pyrimidin-2-yl}-1H- indazol-1- yl)methyl]-3,5- difluorophenoxy} ethanol | ¹H-NMR (400 MHz, DMSO- d₆): δ [ppm] = 0.10 (m, 2H), 0.48 (m, 2H), 0.87 (m, 1H), 1.71 (m, 2H), 1.80 (br. d., 2H), 2.14 (m, 2H), 2.22 (d, 2H), 2.56 (s, 3H), 3.06 (br. d., 2H), 3.17 (m, 1H), 3.67 (td, 2H), 4.00 (t, 2H), 4.89 (t, 1H), 5.71 (s, 2H), 6.82 (m, 2H), 7.27 (dd, 1H), 7.50 (dd, 1H), 7.87 (d, 1H), 8.42 (d, 1H), 8.46 (d, 1H), 8.46 (d, 1H), 8.64 (s, 1H), 9.74 (br. s., 1H). |
| 2-6-24 SM = 2-15-9 18 h at rt reaction | 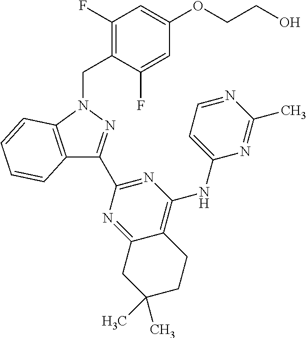 [SMILES: Cc1nccc(Nc2nc(-c3nn(Cc4c(F)cc(OCCO)cc4F)c4ccccc34)nc3c2CCC(C)(C)C3)n1] | 2-{4-[(3-{7,7- dimethyl-4-[(2- methylpyrimidin- 4-yl)amino]- 5,6,7,8- tetrahydroquin- azolin-2-yl}-1H- indazol-1- yl)methyl]-3,5- difluorophenoxy} ethanol | ¹H-NMR (400 MHz, DMSO- d₆): δ [ppm] = 1.00 (s, 6H), 1.61 (m, 2H), 2.67 (br. s., 2H), 2.77 (m, 2H), 2.55 (s, 3H), 3.67 (td, 2H), 4.00 (t, 2H), 4.90 (t, 1H), 5.70 (s, 2H), 6.82 (m, 2H), 7.26 (dd, 1H), 7.49 (dd, 1H), 7.84 (d, 1H), 8.44 (d, 1H), 8.47 (d, 1H), 8.50 (d, 1H), 9.22 (br. s., 1H). |
| 2-6-25 SM = 2-15-10 3 d at rt reaction | 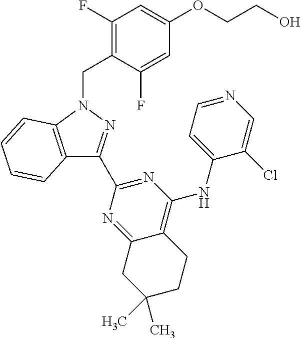 [SMILES: CC1(C)CCc2c(nc(-c3nn(Cc4c(F)cc(OCCO)cc4F)c4ccccc34)nc2Nc2ccncc2Cl)C1] | 2-{4-[(3-{4-[(3- chloropyridin-4- yl)amino]-7,7- dimethyl-5,6,7,8- tetrahydroquin- azolin-2-yl}-1H- indazol-1- yl)methyl]-3,5- difluorophenoxy} ethanol | ¹H-NMR (400 MHz, DMSO- d₆): δ [ppm] = 1.00 (s, 6H), 1.68 (m, 2H), 2.66 (br. s., 2H), 2.74 (m, 2H), 3.67 (td, 2H), 4.00 (t, 2H), 4.89 (t, 1H), 5.69 (s, 2H), 6.81 (m, 2H), 7.18 (dd, 1H), 7.47 (dd, 1H), 7.82 (d, 1H), 8.03 (br. s., 1H), 8.29 (d, 1H), 8.45 (d, 1H), 8.64 (s, 1H), 8.66 (d, 1H). |
| 2-6-26 SM = 2-15-11 19 h at rt reaction | 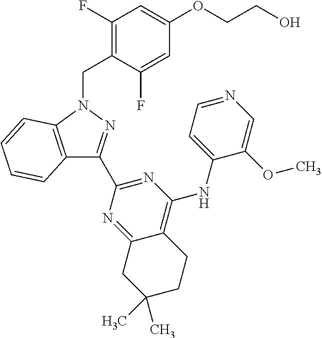 [SMILES: COc1cnccc1Nc1nc(-c2nn(Cc3c(F)cc(OCCO)cc3F)c3ccccc23)nc2c1CCC(C)(C)C2] | 2-{3,5-difluoro-4- [(3-{4-[(3- methoxypyridin- 4-yl)amino]-7,7- dimethyl-5,6,7,8- tetrahydroquin- azolin-2-yl}-1H- indazol-1- yl)methyl] phenoxy}ethanol | ¹H-NMR (400 MHz, DMSO- d₆): δ [ppm] = 1.02 (s, 6H), 1.68 (m, 2H), 2.66 (br. s., 2H), 2.67 (m, 2H), 3.67 (td, 2H), 4.00 (t, 2H), 4.00 (s, 3H), 4.90 (t, 1H), 5.71 (s, 2H), 6.82 (m, 2H), 7.25 (dd, 1H), 7.49 (dd, 1H), 7.74 (br. s., 1H), 7.85 (d, 1H), 8.19 (d, 1H), 8.35 (s, 1H), 8.46 (d, 1H), 8.81 (d, 1H). |
| 2-6-27 SM = 2-12-71 1 h at rt reaction | 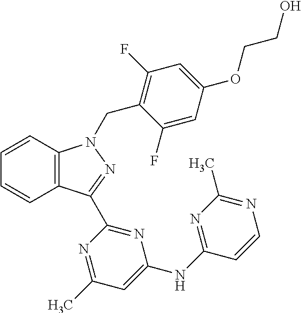 [SMILES: Cc1cc(Nc2ccnc(C)n2)nc(-c2nn(Cc3c(F)cc(OCCO)cc3F)c3ccccc23)n1] | 2-{3,5-difluoro-4- [(3-{4-methyl-6- [(2- methylpyrimidin- 4- yl)amino]pyrimidin- 2-yl}-1H- indazol-1- yl)methyl] phenoxy}ethanol | ¹H-NMR (400MHz, DMSO-d₆): δ [ppm] = 2.52 (s, 3H), 2.56 (s, 3H), 3.68 (q, 2H), 4.01 (t, 2H), 4.89 (t, 1H), 5.73 (s, 2H), 6.77- 6.87 (m, 2H), 7.28-7.35 (m, 1H), 7.46-7.56 (m, 2H), 7.84 (d, 1H), 7.95 (d, 1H), 8.44 (d, 1H), 8.62 (d, 1H), 10.61 (s, 1H). |
| 2-6-28 SM = 2-12-72 1 h at rt reaction | 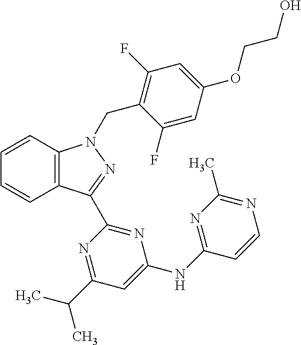 [SMILES: Cc1nccc(Nc2cc(C(C)C)nc(-c3nn(Cc4c(F)cc(OCCO)cc4F)c4ccccc34)n2)n1] | 2-{3,5-difluoro-4- [(3-{4-isopropyl- 6-[(2- methylpyrimidin- 4- yl)amino]pyrimidin- 2-yl}-1H- indazol-1- yl)methyl] phenoxy}ethanol | ¹H-NMR (400MHz, DMSO-d₆): δ [ppm] = 1.35 (d, 6H), 2.55 (s, 3H), 2.96-3.10 (m, 1H), 3.68 (q, 2H), 4.01 (t, 2H), 4.90 (t, 1H), 5.73 (s, 2H), 6.78-6.87 (m, 2H), 7.30-7.37 (m, 1H), 7.48-7.55 (m, 1H), 7.61 (br. s, 1H), 7.84 (d, 1H), 7.96 (br. d, 1H), 8.44 (d, 1H), 8.61 (d, 1H), 10.65 (s, 1H). |
| 2-6-29 SM = 2-12-73 1 h at rt reaction | 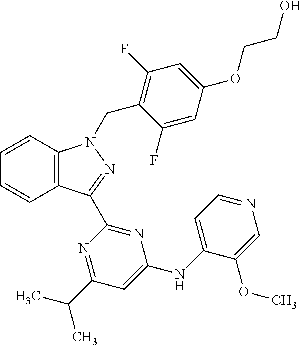 [SMILES: COc1cnccc1Nc1cc(C(C)C)nc(-c2nn(Cc3c(F)cc(OCCO)cc3F)c3ccccc23)n1] | 2-{3,5-difluoro-4- [(3-{4-isopropyl- 6-[(3- methoxypyridin- 4- yl)amino]pyrimidin- 2-yl}-1H- indazol-1- yl)methyl] phenoxy}ethanol | ¹H-NMR (400MHz, DMSO-d₆): δ [ppm] = 1.33 (d, 6H), 2.90- 3.05 (m, 1H), 3.67 (q, 2H), 3.96-4.05 (m, 5H), 4.91 (t, 1H), 5.73 (s, 2H), 6.78-6.87 (m, 2H), 7.15 (s, 1H), 7.28- 7.36 (m, 1H), 7.47-7.56 (m, 1H), 7.89 (d, 1H), 8.14 (d, 1H), 8.30 (s, 1H), 8.56 (d, 1H), 8.96 (d, 1H), 9.16 (s, 1H). |
| 2-6-30 SM = 2-12-74 1 h at rt reaction | 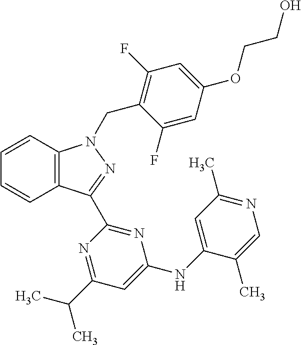 [SMILES: Cc1cc(Nc2cc(C(C)C)nc(-c3nn(Cc4c(F)cc(OCCO)cc4F)c4ccccc34)n2)c(C)cn1] | 2-{4-[(3-{4-[(2,5- dimethylpyridin- 4-yl)amino]-6- isopropylpyrimidin- 2-yl}-1H- indazol-1- yl)methyl]-3,5- difluorophenoxy} ethanol | ¹H-NMR (400MHz, DMSO-d₆): δ [ppm] = 1.33 (d, 6H), 2.26 (s, 3H), 2.43 (s, 3H), 2.90-3.06 (m, 1H), 3.63-3.71 (m, 2H), 4.00 (t, 2H), 4.90 (t, 1H), 5.73 (s, 2H), 6.72-6.84 (m, 2H), 6.95 (s, 1H), 7.23-7.30 (m, 1H), 7.46-7.53 (m, 1H), 7.82 (d, 1H), 8.20 (s, 1H), 8.31 (s, 1H), 8.54 (d, 1H), 8.84 (s, 1H). |
| 2-6-31 SM = 2-12-76 1 h at rt reaction | 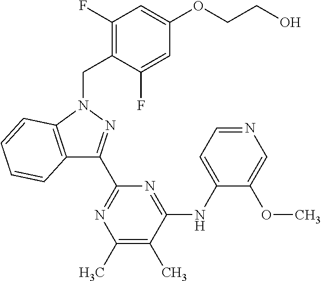 [SMILES: COc1cnccc1Nc1nc(-c2nn(Cc3c(F)cc(OCCO)cc3F)c3ccccc23)nc(C)c1C] | 2-{3,5-difluoro-4- [(3-{4-[(3- methoxypyridin- 4-yl)amino]-5,6- dimethylpyrimidin- 2-yl}-1H-indazol- 1- yl)methyl] phenoxy}ethanol | ¹H-NMR (400MHz, DMSO-d₆): δ [ppm] = 2.24 (s, 3H), 2.52 (s, 3H), 3.64-3.71 (m, 2H), 3.96- 4.06 (m, 5H), 4.91 (br. s, 1H), 5.71 (s, 2H), 6.78-6.88 (m, 2H), 7.22-7.28 (m, 1H), 7.45-7.53 (m, 1H), 7.81- 7.89 (m, 2H), 8.18 (d, 1H), 8.34 (s, 1H), 8.46 (d, 1H), 8.75 (d, 1H). |
| 2-6-32 SM = 2-12-75 1 h at rt reaction | 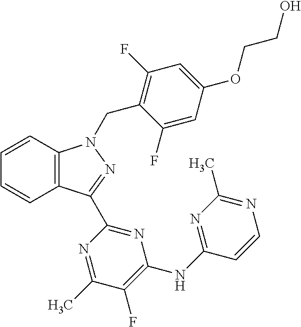 [SMILES: Cc1nccc(Nc2nc(-c3nn(Cc4c(F)cc(OCCO)cc4F)c4ccccc34)nc(C)c2F)n1] | 2-{3,5-difluoro-4- [(3-{5-fluoro-4- methyl-6-[(2- methylpyrimidin- 4- yl)amino]pyrimidin- 2-yl}-1H- indazol-1- yl)methyl] phenoxy}ethanol | ¹H-NMR (400MHz, DMSO-d₆): δ [ppm] = 2.52-2.54 (m, 3H), 2.57 (s, 3H), 3.67 (q, 2H), 4.01 (t, 2H), 4.90 (t, 1H), 5.72 (s, 2H), 6.77-6.89 (m, 2H), 7.27- 7.35 (m, 1H), 7.47-7.55 (m, 1H), 7.86 (d, 1H), 8.38 (d, 1H), 8.48-8.55 (m, 2H), 10.46 (br. s, 1H). |
| 2-6-33 SM = 2-12-77 | 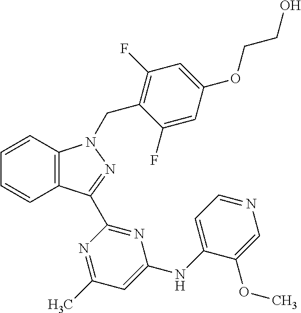 [SMILES: COc1cnccc1Nc1cc(C)nc(-c2nn(Cc3c(F)cc(OCCO)cc3F)c3ccccc23)n1] | 2-{3,5-difluoro-4- [(3-{4-[(3- methoxypyridin- 4-yl)amino]-6- methylpyrimidin- 2-yl}-1H-indazol- 1- yl)methyl] phenoxy}ethanol | ¹H-NMR (400MHz, DMSO-d₆): δ [ppm] = 2.46 (s, 3H), 3.68 (br. s, 2H), 3.96-4.06 (m, 5H), 4.91 (br. s, 1H), 5.72 (s, 2H), 6.75-6.88 (m, 2H), 7.07 (s, 1H), 7.22-7.33 (m, 1H), 7.43-7.55 (m, 1H), 7.87 (d, 1H), 8.14 (d, 1H), 8.31 (s, 1H), 8.53 (d, 1H), 8.84 (d, 1H), 9.18 (s, 1H). |
| 2-6-34 SM = 2-2-47 | 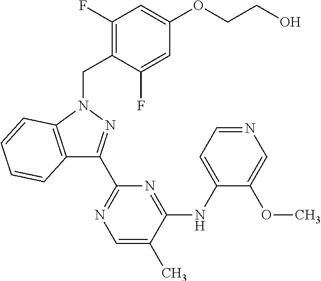 [SMILES: COc1cnccc1Nc1nc(-c2nn(Cc3c(F)cc(OCCO)cc3F)c3ccccc23)ncc1C] | 2-{3,5-difluoro-4- [(3-{4-[(3- methoxypyridin- 4-yl)amino]-5- methylpyrimidin- 2-yl}-1H-indazol- 1- yl)methyl] phenoxy}ethanol | ¹H-NMR (400MHz, DMSO-d₆): δ [ppm] = 2.30 (s, 3H), 3.64- 3.72 (m, 2H), 3.97-4.04 (m, 5H), 4.91 (br. s, 1H), 5.71 (s, 2H), 6.78-6.86 (m, 2H), 7.21- 7.28 (m, 1H), 7.45-7.53 (m, 1H), 7.83-7.90 (m, 2H), 8.21 (d, 1H), 8.36-8.43 (m, 2H), 8.46 (d, 1H), 8.75 (d, 1H). |
| 2-6-35 SM = 2-12-78 | 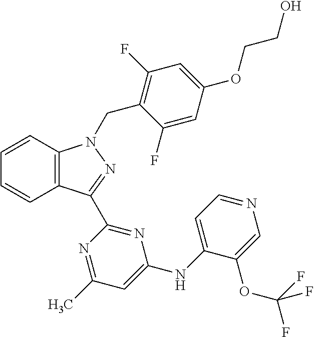 [SMILES: Cc1cc(Nc2ccncc2OC(F)(F)F)nc(-c2nn(Cc3c(F)cc(OCCO)cc3F)c3ccccc23)n1] | 2-(3,5-difluoro-4- {[3-(4-methyl-6- {[3- (trifluoromethoxy) pyridin-4- yl]amino} pyrimidin-2-yl)-1H- indazol-1- yl]methyl} phenoxy)ethanol | ¹H-NMR (400 MHz, DMSO- d₆): δ [ppm] = 2.50 (s, 3H), 3.67 (td, 2H), 4.01 (t, 2H), 4.89 (t, 1H), 5.72 (s, 2H), 6.83 (m, 2H), 7.10 (s, 1H), 7.29 (dd, 1H), 7.51 (dd, 1H), 7.87 (d, 1H), 8.43 (d, 1H), 8.51 (d, 1H), 8.57 (br. s., 1H), 9.04 (d, 1H), 9.74 (br. s., 1H). |
| 2-6-36 SM = 2-2-48 | 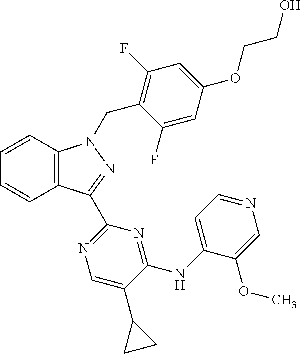 [SMILES: COc1cnccc1Nc1nc(-c2nn(Cc3c(F)cc(OCCO)cc3F)c3ccccc23)ncc1C1CC1] | 2-{4-[(3-{5- cyclopropyl-4-[(3- methoxypyridin- 4- yl)amino]pyrimidin- 2-yl}-1H- indazol-1- yl)methyl]-3,5- difluorophenoxy} ethanol | ¹H-NMR (400MHz, DMSO-d₆): δ [ppm] = 0.72-0.79 (m, 2H), 1.04-1.11 (m, 2H), 1.84- 1.95 (m, 1H), 3.67 (q, 2H), 4.01 (t, 2H), 4.06 (s, 3H), 4.90 (t, 1H), 5.72 (s, 2H), 6.79- 6.89 (m, 2H), 7.29 (t, 1H), 7.51 (t, 1H), 7.89 (d, 1H), 8.22 (d, 1H), 8.36 (d, 2H), 8.43-8.49 (m, 2H), 8.97 (d, 1H). |
| 2-6-37 SM = 2-12-39 | 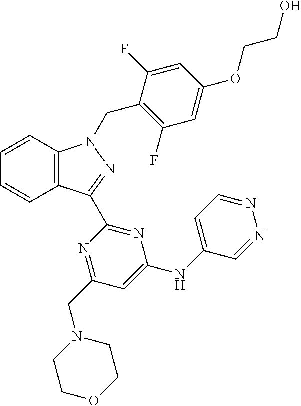 [SMILES: OCCOc1cc(F)c(Cn2nc(-c3nc(CN4CCOCC4)cc(Nc4ccnnc4)n3)c3ccccc32)c(F)c1] | 2-[3,5-difluoro-4- ({3-[4- (morpholin-4- ylmethyl)-6- (pyridazin-4- ylamino)pyrimidin- 2-yl]-1H- indazol-1- yl}methyl) phenoxy]ethanol | ¹H-NMR (400MHz, DMSO-d₆): δ [ppm] = 2.55 (br. s., 4H), 3.63-3.73 (m, 8H), 4.02 (t, 2H), 4.90 (t, 1H), 5.74 (s, 2H), 6.80-6.89 (m, 2H), 7.04 (s, 1H), 7.29-7.36 (m, 1H), 7.49- 7.56 (m, 1H), 7.89 (d, 1H), 8.53 (d, 1H), 8.60-8.65 (m, 1H), 8.96 (d, 1H), 9.34-9.39 (m, 1H), 10.40 (s, 1H). |
| 2-6-38 SM = 2-12-43 | 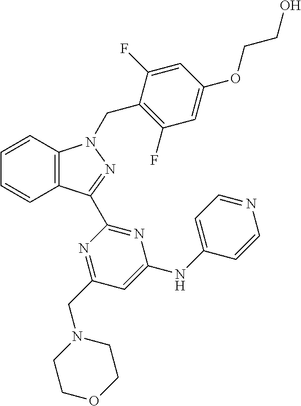 [SMILES: OCCOc1cc(F)c(Cn2nc(-c3nc(CN4CCOCC4)cc(Nc4ccncc4)n3)c3ccccc32)c(F)c1] | 2-[3,5-difluoro-4- ({3-[4- (morpholin-4- ylmethyl)-6- (pyridin-4- ylamino)pyrimidin- 2-yl]-1H- indazol-1- yl}methyl) phenoxy]ethanol | ¹H-NMR (400MHz, DMSO-d₆): δ [ppm] = .53-2.59 (m, 4H), 3.61-3.73 (m, 8H), 4.01 (t, 2H), 4.91 (t, 1H), 5.73 (s, 2H), 6.80-6.88 (m, 2H), 6.98 (s, 1H), 7.27-7.34 (m, 1H), 7.48- 7.55 (m, 1H), 7.89 (d, 1H), 7.92-7.98 (m, 2H), 8.39- 8.45 (m, 2H), 8.55 (d, 1H), 10.10 (s, 1H). |
| 2-6-39 SM = 2-20-16 | 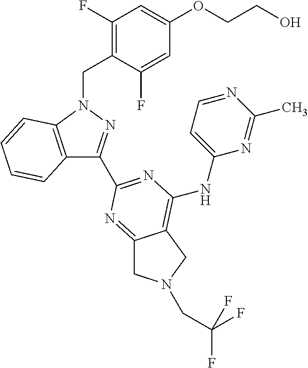 [SMILES: Cc1nccc(Nc2nc(-c3nn(Cc4c(F)cc(OCCO)cc4F)c4ccccc34)nc3c2CN(CC(F)(F)F)C3)n1] | 2-{3,5-difluoro-4- [(3-{4-[(2- methylpyrimidin- 4-yl)amino]-6- (2,2,2- trifluoroethyl)- 6,7-dihydro-5H- pyrrolo[3,4- d]pyrimidin-2-yl}- 1H-indazol-1- yl)methyl] phenoxy}ethanol | ¹H-NMR (400 MHz, DMSO- d₆): δ [ppm] = 2.51 (s, 3H), 3.61 (q, 2H), 3.67 (br. t., 2H), 4.00 (t, 2H), 4.18 (br. s., 2H), 4.22 (br. s., 2H), 4.91 (m, 1H), 5.71 (s, 2H), 6.82 (m, 2H), 7.27 (dd, 1H), 7.49 (dd, 1H), 7.85 (d, 1H), 8.20 (m, 1H), 8.39 (m, 1H), 8.51 (d, 1H), 10.38 (m, 1H). |
| 2-6-40 SM = 2-20-18 | 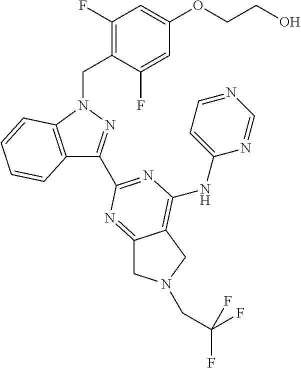 [SMILES: OCCOc1cc(F)c(Cn2nc(-c3nc4c(c(Nc5ccncn5)n3)CN(CC(F)(F)F)C4)c3ccccc32)c(F)c1] | 2-[3,5-difluoro-4- ({3-[4-(pyrimidin- 4-ylamino)-6- (2,2,2- trifluoroethyl)- 6,7-dihydro-5H- pyrrolo[3,4- d]pyrimidin-2-yl]- 1H-indazol-1- yl}methyl) phenoxy]ethanol | ¹H-NMR (600 MHz, DMSO- d₆): δ [ppm] = 3.62 (q, 2H), 3.67 (dt, 2H), 4.01 (t, 2H), 4.22 (br. s., 2H), 4.28 (br. s., 2H), 4.87 (t, 1H), 5.72 (s, 2H), 6.83 (m, 2H), 7.30 (dd, 1H), 7.51 (dd, 1H), 7.87 (d, 1H), 8.51 (d, 1H), 8.55 (d, 1H), 8.57 (d, 1H), 8.86 (s, 1H), 10.43 (br. s., 1H). |
| 2-6-41 SM = 2-20-19 | 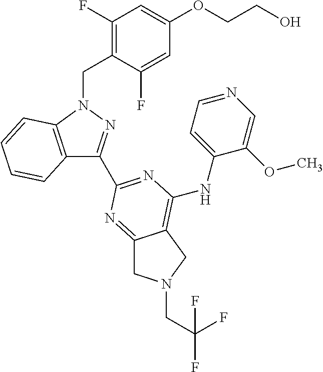 [SMILES: COc1cnccc1Nc1nc(-c2nn(Cc3c(F)cc(OCCO)cc3F)c3ccccc23)nc2c1CN(CC(F)(F)F)C2] | 2-{3,5-difluoro-4- [(3-{4-[(3- methoxypyridin- 4-yl)amino]-6- (2,2,2- trifluoroethyl)- 6,7-dihydro-5H- pyrrolo[3,4- d]pyrimidin-2-yl}- 1H-indazol-1- yl)methyl] phenoxy}ethanol | ¹H-NMR (400 MHz, DMSO- d₆): δ [ppm] = 3.64 (q, 2H), 3.67 (dt, 2H), 4.01 (t, 2H), 4.19 (br. s, 2H), 4.24 (br. s., 2H), 4.91 (t, 1H), 3.94 (s, 3H), 5.70 (s, 2H), 6.81 (m, 2H), 7.19 (dd, 1H), 7.47 (dd, 1H), 7.83 (d, 1H), 8.18 (d, 1H), 8.32 (d, 1H), 8.33 (d, 1H), 8.38 (br. s, 1H), 8.49 (s, 1H). |
| 2-6-42 SM = 2-15-12 | 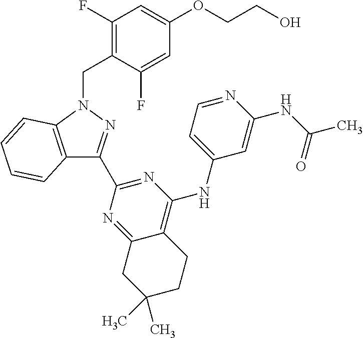 [SMILES: CC(=O)Nc1cc(Nc2nc(-c3nn(Cc4c(F)cc(OCCO)cc4F)c4ccccc34)nc3c2CCC(C)(C)C3)ccn1] | N-{4-[(2-{1-[2,6- difluoro-4-(2- hydroxyethoxy) benzyl]-1H- indazol-3-yl}-7,7- dimethyl-5,6,7,8- tetrahydroquin- azolin-4- yl)amino]pyridin- 2-yl}acetamide | ¹H-NMR (400 MHz, DMSO- d₆): δ [ppm] = 1.02 (s, 6H), 1.64 (m, 2H), 2.08 (s, 3H), 2.64 (br. s., 2H), 2.72 (m, 2H), 3.67 (td, 2H), 4.00 (t, 2H), 4.89 (t, 1H), 5.69 (s, 2H), 6.79 (m, 2H), 7.20 (dd, 1H), 7.46 (dd, 1H), 7.80 (d, 1H), 8.14 (d, 1H), 8.23 (dd, 1H), 8.26 (d, 1H), 8.47 (d, 1H), 8.90 (br. s., 1H), 10.33 (br. s., 1H). |
| 2-6-43 SM = 2-15-13 | 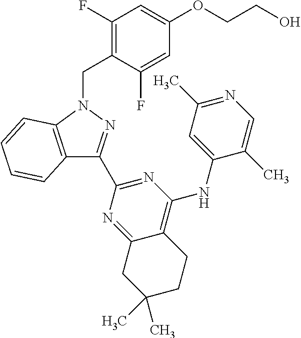 [SMILES: Cc1cc(Nc2nc(-c3nn(Cc4c(F)cc(OCCO)cc4F)c4ccccc34)nc3c2CCC(C)(C)C3)c(C)cn1] | 2-{4-[(3-{4-[(2,5- dimethylpyridin- 4-yl)amino]-7,7- dimethyl-5,6,7,8- tetrahydroquin- azolin-2-yl}-1H- indazol-1- yl)methyl]-3,5- difluorophenoxy} ethanol | ¹H-NMR (400 MHz, DMSO- d₆): δ [ppm] = 1.03 (s, 6H), 1.67 (m, 2H), 2.20 (s, 3H), 2.44 (s, 3H), 2.61 (br. s., 2H), 2.70 (m, 2H), 3.67 (td, 2H), 4.00 (t, 2H), 4.91 (t, 1H), 5.67 (s, 2H), 6.77 (m, 2H), 7.10 (dd, 1H), 7.43 (dd, 1H), 7.76 (d, 1H), 7.85 (br. s., 1H), 7.87 (s, 1H), 8.19 (d, 1H), 8.28 (s, 1H). |
| 2-6-44 SM = 2-12-70 | 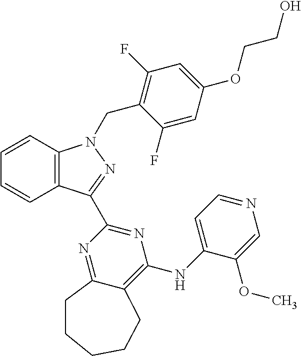 [SMILES: COc1cnccc1Nc1nc(-c2nn(Cc3c(F)cc(OCCO)cc3F)c3ccccc23)nc2c1CCCCC2] | 2-{3,5-difluoro-4- [(3-{4-[(3- methoxypyridin- 4-yl)amino]- 6,7,8,9- tetrahydro-5H- cyclohepta[d] pyrimidin-2-yl}-1H- indazol-1- yl)methyl] phenoxy}ethanol | ¹H-NMR (400MHz, DMSO-d₆): δ [ppm] = 1.58-1.73 (m, 4H), 1.83-1.93 (m, 2H), 2.78- 2.85 (m, 2H), 2.99-3.08 (m, 2H), 3.63-3.71 (m, 2H), 3.97 (s, 3H), 4.01 (t, 2H), 4.91 (t, 1H), 5.70 (s, 2H), 6.77-6.86 (m, 2H), 7.18-7.25 (m, 1H), 7.44-7.52 (m, 1H), 7.83 (d, 1H), 8.12 (s, 1H), 8.17 (d, 1H), 8.34 (s, 1H), 8.40 (d, 1H), 8.55 (d, 1H). |

### Example 2-7-1

### Preparation of 2-[1-(4-ethoxy-2,6-difluorobenzyl)-1H-indazol-3-yl]-5-[2-(S-methylsulfonimidoyl)ethyl]-N-(pyridin-4-yl)pyrimidin-4-amine

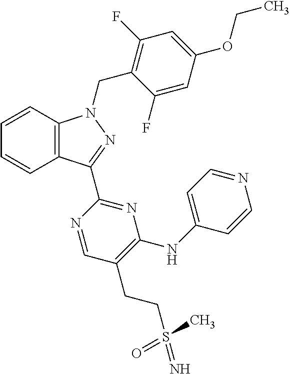 [SMILES: CCOc1cc(F)c(Cn2nc(-c3ncc(CC[S@](C)(=N)=O)c(Nc4ccncc4)n3)c3ccccc32)c(F)c1]

38 mg N-[(2-{2-[1-(4-ethoxy-2,6-difluorobenzyl)-1H-indazol-3-yl]-4-(pyridin-4-ylamino)pyrimidin-5-yl}ethyl)(methyl)oxido-lambda6-sulfanylidene]-2,2,2-trifluoroacetamide 2-8-1 (0.05 mmol, 1.0 eq.) was suspended in 1.2 mL methanol and treated with 39 mg (30.5 mmol, 5 eq) potassium carbonate. It was stirred at rt for 2 hours. The reaction mixture was diluted with ethyl acetated and brine and stirred for 10 minutes.

It was extracted once with ethyl acetate and twice with dichloromethane/2-propanole. The organic layers were dried with a water resistant filter and concentrated under reduced pressure. The crude product was purified by HPLC chromatography to provide 7 mg of the target compound in 99% purity, 21%

¹H-NMR (300 MHz, DMSO-d₆): δ [ppm]=1.28 (t, 3H), 3.01 (s, 3H), 3.13-3.23 (m, 2H), 3.39-3.51 (m, 2H), 3.98-4.11 (m, 3H), 5.70 (s, 2H), 6.74-6.84 (m, 2H), 7.25 (t, 1H), 7.49 (t, 1H), 7.86 (d, 1H), 8.03 (d, 2H), 8.36-8.57 (m, 4H), 9.33 (s, 1H).

### Example 2-8-1

### Preparation of N-[(2-{2-[1-(4-ethoxy-2,6-difluorobenzyl)-1H-indazol-3-yl]-4-(pyridin-4-ylamino)pyrimidin-5-yl}ethyl)(methyl)oxido-A6-sulfanylidene]-2,2,2-trifluoroacetamide

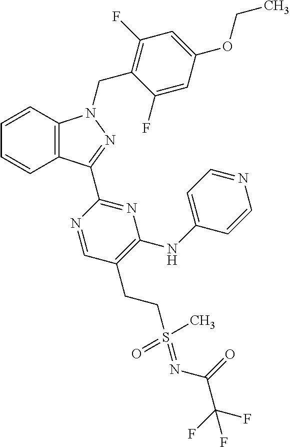 [SMILES: CCOc1cc(F)c(Cn2nc(-c3ncc(CCS(C)(=O)=NC(=O)C(F)(F)F)c(Nc4ccncc4)n3)c3ccccc32)c(F)c1]

215 mg 2-[1-(4-ethoxy-2,6-difluorobenzyl)-1H-indazol-3-yl]-5-[2-(methylsulfinyl)ethyl]-N-(pyridin-4-yl)pyrimidin-4-amine 2-1-1 (0.33 mmol, 1.0 eq, 85% purity), 75 mg 2,2,2-trifluoroacetamide (0.66 mmol, 2.0 eq.), 161 mg lodbenzoldiacetat (0.50 mmol, 1.5 eq.), 7 mg rhodium(II) acetate, dimer (0.03 mmol, 0.1 eq.) and 54 mg magnesium oxide (1.33 mmol, 4.0 eq) were combined in 10 mL dichloromethane. It was stirred at rt over night. All reagents were added again and it was stirred at rt for 3 hours. All unsolved compounds were filtered off and washed with dichloromethane. The filtrate was concentrated under reduced pressure and purified by flash chromatography to provide 38 mg of the target compound, 17%.

LC-MS (Method 6):

retention time: 1.38 min

MS ES+: 660 [M+H]⁺

### Example 2-9-1

### Preparation of 5-(azetidin-3-yl)-2-[1-(4-ethoxy-2,6-difluorobenzyl)-1H-indazol-3-yl]-N-(pyridin-4-yl)pyrimidin-4-amine, salt with hydrochloric acid

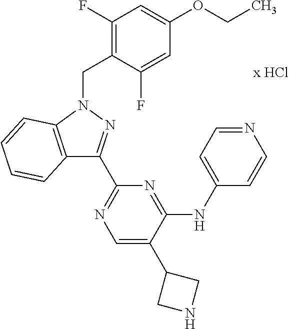 [SMILES: CCOc1cc(F)c(Cn2nc(-c3ncc(C4CNC4)c(Nc4ccncc4)n3)c3ccccc32)c(F)c1]

154 mg tert-butyl 3-{2-[1-(4-ethoxy-2,6-difluorobenzyl)-1H-indazol-3-yl]-4-(pyridin-4-ylamino)pyrimidin-5-yl}azetidine-1-carboxylate 2-2-6 (0.25 mmol, 1.0 eq.) was dissolved in 1.4 mL ethanol and 1.6 mL dichloromethane and treated with 1.2 mL hydrochloric acid (4N in dioxane, 5.02 mmol, 20 eq.). It was stirred at rt over night. Precipitated crystals were filtered off, washed with ethanol and dried in the vacuum oven to provide 109 mg of the target compound in 95% purity: 74% ¹H-NMR (400 MHz, DMSO-d₆ with 3 drops of TFA-d): δ [ppm]=1.27 (t, 3H), 3.21-3.38 (m, 2H), 3.96-4.09 (m, 3H), 4.29-4.50 (m, 2H), 5.75 (s, 2H), 6.68-6.85 (m, 2H), 7.33-7.41 (m, 1H), 7.50-7.59 (m, 1H), 7.85 (d, 1H), 8.39-8.52 (m, 3H), 8.79 (d, 2H), 8.85 (d, 1H).

The following compounds were prepared according to the same procedure from the indicated starting materials (SM=starting material):

| | | | |
|---|---|---|---|
| 2-9-2 SM = 2-2-7 | 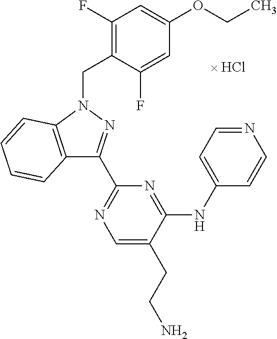 [SMILES: CCOc1cc(F)c(Cn2nc(-c3ncc(CCN)c(Nc4ccncc4)n3)c3ccccc32)c(F)c1] | 5-(2-aminoethyl)- 2-[1-(4-ethoxy-2,6- difluorobenzyl)- 1H-indazol-3-yl]- N-(pyridin-4- yl)pyrimidin-4- amine, salt with hydrochloric acid | ¹H-NMR (300 MHz, DMSO-d₆): δ [ppm] = 1.27 (t, 3H), 3.04- 3.22 (m, 2H), 3.22-3.41 (m, 2H), 4.02 (q, 2H), 5.74 (s, 2H), 6.76-6.85 (m, 2H), 7.30 (t, 1H), 7.52 (t, 1H), 7.88 (d, 1H), 8.11 (br. s., 2H), 8.43 (d, 1H), 8.62 (d, 2H), 8.72 (s, 1H), 8.88 (d, 2H), 10.76 (br.s., 1H), 14.68 (br. s., 1H). |
| 2-9-3 SM = 2-2-30 | 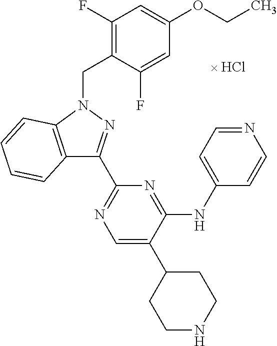 [SMILES: CCOc1cc(F)c(Cn2nc(-c3ncc(C4CCNCC4)c(Nc4ccncc4)n3)c3ccccc32)c(F)c1] | 2-[1-(4-ethoxy-2,6- difluorobenzyl)- 1H-indazol-3-yl]- 5-(piperidin-4-yl)- N-(pyridin-4- yl)pyrimidin-4- amine, salt with hydrochloric acid | ¹H-NMR (400 MHz, DMSO-d₆): δ [ppm] = 1.26 (t, 3H), 1.91- 2.13 (m, 4H), 3.07-3.26 (m, 2H), 3.28-3.45 (m, 2H), 4.02 (q, 2H), 5.73 (s, 2H), 6.79 (d, 2H), 7.28 (t, 1H), 7.51 (t, 1H), 7.86 (d, 1H), 8.39 (d, 1H), 8.60 (d, 2H), 8.66 (s, 1H), 8.88 (d, 2H), 9.05 (d, 1H), 9.18 (br. s., 1H), 11.24 (br. s., 1H). |
| 2-9-4 SM = 2-2-21 | 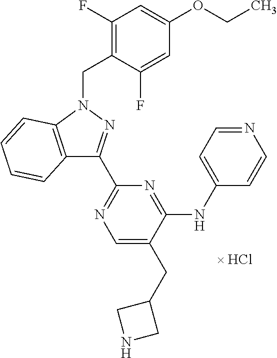 [SMILES: CCOc1cc(F)c(Cn2nc(-c3ncc(CC4CNC4)c(Nc4ccncc4)n3)c3ccccc32)c(F)c1] | 5-(azetidin-3- ylmethyl)-2-[1-(4- ethoxy-2,6- difluorobenzyl)- 1H-indazol-3-yl]- N-(pyridin-4- yl)pyrimidin-4- amine, salt with hydrochloric acid | ¹H-NMR (400 MHz, DMSO-d₆): δ [ppm] = 1.29 (t, 3H), 2.57- 2.65 (m, 1H), 2.87-3.11 (m, 2H), 3.30-3.43 (m, 2H), 3.70- 3.89 (m, 2H), 4.04 (q, 2H), 5.75 (s, 2H), 6.77-6.89 (m, 2H), 7.26-7.36 (m, 1H), 7.49- 7.59 (m, 1H), 7.90 (d, 1H), 8.20 (br. s, 2H), 8.45 (d, 1H), 8.62 (d, 2H), 8.72 (s, 1H), 8.77- 8.86 (m, 2H), 10.81 (s, 1H). |
| 2-9-5 SM = 2-12-11 | 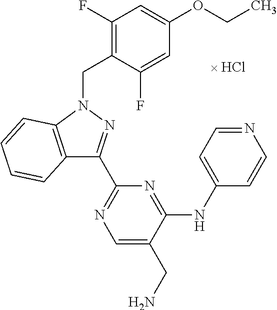 [SMILES: CCOc1cc(F)c(Cn2nc(-c3ncc(CN)c(Nc4ccncc4)n3)c3ccccc32)c(F)c1] | 5-(aminomethyl)- 2-[1-(4-ethoxy- 2,6-difluorobenzyl)- 1H-indazol-3-yl]- N-(pyridin-4- yl)pyrimidin-4- amine, salt with hydrochloric acid | ¹H-NMR (600 MHz, DMSO-d₆): δ [ppm] = 1.28 (t, 3H), 4.03 (q, 2H), 4.42 (d, 2H), 5.64-5.83 (m, 2H), 6.81 (d, 2H), 7.32 (t, 1H), 7.47-7.62 (m, 1H), 7.89 (d, 1H), 8.37 (br. s., 3H), 8.42 (d, 1H), 8.57-8.75 (m, 4H), 8.88 (s, 1H), 10.92 (br. s., 1H), 14.46 (br.s, 1H). |
| 2-9-6 SM = 2-12-10 | 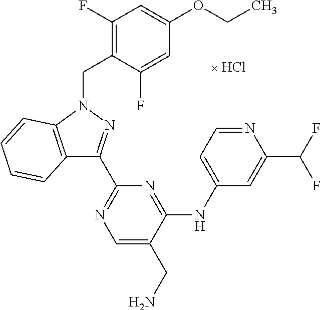 [SMILES: CCOc1cc(F)c(Cn2nc(-c3ncc(CN)c(Nc4ccnc(C(F)F)c4)n3)c3ccccc32)c(F)c1] | 5-(aminomethyl)- N-[2-(difluoro- methyl)pyridin-4- yl]-2-[1-(4-ethoxy- 2,6-difluorobenzyl)- 1H-indazol-3- yl]pyrimidin-4- amine, salt with hydrochloric acid | ¹H-NMR (400 MHz, DMSO-d₆): δ [ppm] = 1.29 (t, 3H), 4.05 (q, 2H), 4.26-4.42 (m, 2H), 5.61- 5.81 (m, 2H), 6.75-6.88 (m, 2H), 6.95 (t, 1H), 7.29 (t, 1H), 7.48-7.60 (m, 1H), 7.88 (d, 1H), 8.20 (d, 1H), 8.33 (br. s., 3H), 8.42 (d, 1H), 8.51 (d, 1H), 8.58 (d, 1H), 8.72 (s, 1H), 9.86 (br. s., 1H). |
| 2-9-7 SM = 2-2-23 | 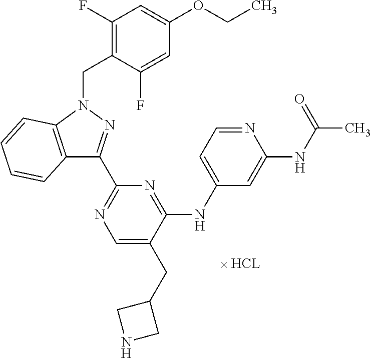 [SMILES: CCOc1cc(F)c(Cn2nc(-c3ncc(CC4CNC4)c(Nc4ccnc(NC(C)=O)c4)n3)c3ccccc32)c(F)c1] | N-[4-({5-(azetidin- 3-ylmethyl)-2-[1-(4- ethoxy-2,6- difluorobenzyl)- 1H-indazol-3- yl]pyrimidin-4- yl}amino)pyridin- 2-yl]acetamide, salt with hydrochloric acid | ¹H NMR (400 MHz, DMSO-d₆) δ ppm 1.27 (t, 3 H) 2.21 (s, 3 H) 2.80-3.10 (m, 2 H) 3.21- 3.47 (m, 2 H) 3.71-3.93 (m, 2 H) 3.93-4.10 (m, 2 H) 5.73 (s, 2 H) 6.77 (s, 1 H) 6.80 (s, 1 H) 7.25 (t, 1 H) 7.52 (t, 1 H) 7.76- 7.93 (m, 1 H) 8.01-8.12 (m, 1 H) 8.16-8.38 (m, 4 H) 8.42 (s, 1 H) 8.52-8.75 (m, 1 H) 10.90 (s, 1 H) 11.96 (s, 1 H). |
| 2-9-8 SM = 2-2-24 | 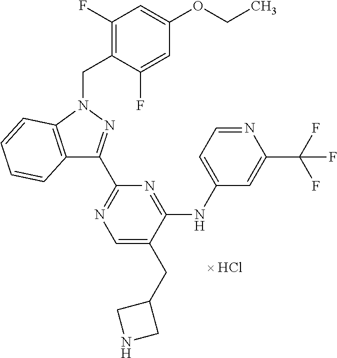 [SMILES: CCOc1cc(F)c(Cn2nc(-c3ncc(CC4CNC4)c(Nc4ccnc(C(F)(F)F)c4)n3)c3ccccc32)c(F)c1] | 5-(azetidin-3- ylmethyl)-2-[1-(4- ethoxy-2,6- difluorobenzyl)- 1H-indazol-3-yl]- N-[2-trifluoro- methyl)pyridin-4- yl]pyrimidin-4- amine, salt with hydrochloric acid | ¹H-NMR (400 MHz, DMSO-d₆): δ [ppm] = 1.29 (t, 3H), 2.85- 3.08 (m, 4H), 3.87 (dd, 2H), 4.04 (q, 2H), 5.82 (s, 2H), 6.78 (d, 2H), 7.26 (d, 1H), 7.56 (d, 1H), 7.90 (d, 2H), 8.06-8.23 (m, 2H), 8.36 (s, 1H), 8.52 (s, 1H), 8.80 (d, 1H). 1H not assigned |
| 2-9-9 SM = 2-2-25 | 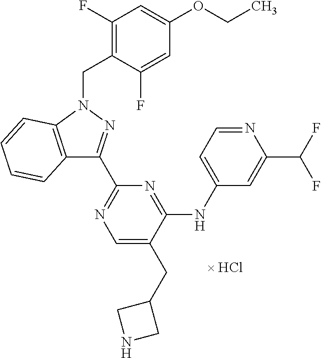 [SMILES: CCOc1cc(F)c(Cn2nc(-c3ncc(CC4CNC4)c(Nc4ccnc(C(F)F)c4)n3)c3ccccc32)c(F)c1] | 5-(azetidin-3- ylmethyl)-N-[2- (difluorometh- yl)pyridin-4-yl]-2- [1-(4-ethoxy-2,6- difluorobenzyl)- 1H-indazol-3- yl]pyrimidin-4- amine, salt with hydrochloric acid | ¹H-NMR (400 MHz, DMSO-d₆): δ [ppm] = 1.29 (t, 3H), 2.80- 3.04 (m, 3H), 3.85 (m, 2H), 4.05 (q, 2H), 5.73 (s, 2H), 6.80 (d, 2H), 7.26 (t, 1H), 7.51 (dt, 1H), 7.87 (d, 1H), 8.11 (d, 1H), 8.44 (d, 1H), 8.49 (dd, 1H), 8.53 (s, 1H), 8.55 (d, 1H), 9.16 (s, 1H), 4H's not assigned. |
| 2-9-10 SM = 2-2-26 | 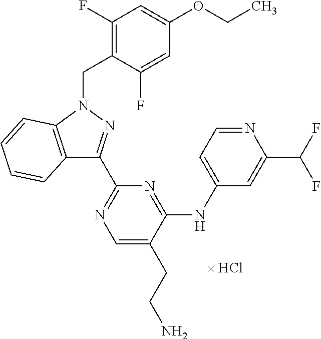 [SMILES: CCOc1cc(F)c(Cn2nc(-c3ncc(CCN)c(Nc4ccnc(C(F)F)c4)n3)c3ccccc32)c(F)c1] | 5-(2-aminoethyl)- N-[2-(difluorometh- yl)pyridin-4-yl]-2- [1-(4-ethoxy-2,6- difluorobenzyl)- 1H-indazol-3- yl]pyrimidin-4- amine, salt with hydrochloric acid | ¹H-NMR (400 MHz, DMSO-d₆): δ [ppm] = 1.29 (t, 3H), 3.16 (d, 2H), 3.22-3.38 (m, 2H), 4.04 (q, 2H), 5.81 (s, 2H), 6.70- 6.84 (m, 2H), 7.01 (t, 1H), 7.28 (t, 1H), 7.51-7.62 (m, 1H), 7.89 (d, 1H), 8.11-8.28 (m, 4H), 8.28-8.42 (m, 2H), 8.56 (s, 1H), 8.68 (d, 1H), 10.56 (br. s., 1H). |
| 2-9-11 SM = 2-2-27 | 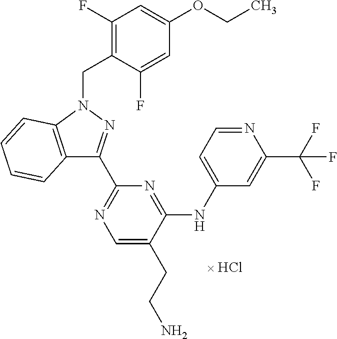 [SMILES: CCOc1cc(F)c(Cn2nc(-c3ncc(CCN)c(Nc4ccnc(C(F)(F)F)c4)n3)c3ccccc32)c(F)c1] | 5-(2-aminoethyl)- 2-[1-(4-ethoxy-2,6- difluorobenzyl)- 1H-indazol-3-yl]- N-[2-(trifluorometh- yl)pyridin-4- yl]pyrimidin-4- amine, salt with hydrochloric acid | ¹H-NMR (400 MHz, DMSO-d₆): δ [ppm] = 1.29 (t, 3H), 3.14 (d, 2H), 3.21-3.32 (m, 2H), 4.04 (q, 2H), 5.78 (s, 2H), 6.79 (d, 2H), 7.26 (t, 1H), 7.49-7.58 (m, 1H), 7.87 (d, 1H), 8.10 (br. s., 3H), 8.29 (d, 1H), 8.51 (d, 1H), 8.56 (s, 1H), 8.63 (d, 1H), 8.69 (d, 1H), 10.19 (br. s., 1H). |
| 2-9-12 SM = 2-2-28 | 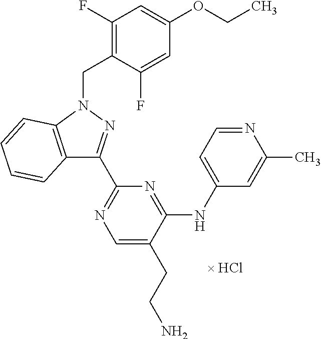 [SMILES: CCOc1cc(F)c(Cn2nc(-c3ncc(CCN)c(Nc4ccnc(C)c4)n3)c3ccccc32)c(F)c1] | 5-(2-aminoethyl)- 2-[1-(4-ethoxy-2,6- difluorobenzyl)- 1H-indazol-3-yl]- N-(2-methylpyridin- 4-yl)pyrimidin-4- amine, salt with hydrochloric acid | ¹H-NMR (400 MHz, DMSO-d₆): δ [ppm] = 1.29 (t, 3H), 2.65 (s, 3H), 3.14 (d, 2H), 3.21-3.28 (m, 2H), 4.04 (q, 2H), 5.77 (s, 2H), 6.80 (s, 1H), 6.77 (s, 1H), 7.33 (t, 1H), 7.54 (t, 1H), 7.88 (d, 1H), 7.98 (br. s., 3H), 8.41- 8.55 (m, 3H), 8.72 (s, 1H), 8.76 (br. s., 1H), 10.38 (br. s., 1H), 14.28-14.83 (m, 1H). |
| 2-9-13 SM = 2-2-29 | 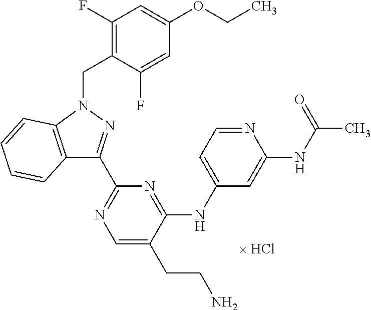 [SMILES: CCOc1cc(F)c(Cn2nc(-c3ncc(CCN)c(Nc4ccnc(NC(C)=O)c4)n3)c3ccccc32)c(F)c1] | N-[4-({5-(2- aminoethyl)-2-[1- (4-ethoxy-2,6- difluorobenzyl)- 1H-indazol-3- yl]pyrimidin-4- yl}amino)pyridin- 2-yl]acetamide, salt with hydrochloric acid | ¹H NMR (400 MHz, DMSO-d₆) δ ppm 1.27 (t, 3 H) 2.22 (s, 3 H) 3.11-3.14 (d, 2 H) 3.26- 3.29 (d, 2 H) 4.02 (q, 2 H) 5.75 (s, 2 H) 6.77 (s, 1 H) 6.80 (s, 1 H) 7.26 (t, 1 H) 7.52 (t, 1 H) 7.88 (d, 2 H) 8.08 (s, 1 H) 8.17 (s, 1 H) 8.22-8.41 (m, 2 H) 8.48 (s, 1 H) 8.56-8.72 (m, 1 H) 10.84 (s, 1 H) 11.96 (s, 1 H). |
| 2-9-14 SM = 2-15-3 | 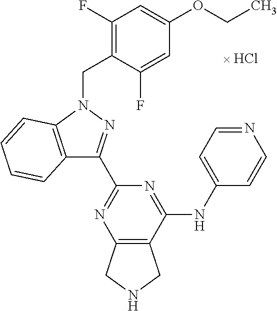 [SMILES: CCOc1cc(F)c(Cn2nc(-c3nc4c(c(Nc5ccncc5)n3)CNC4)c3ccccc32)c(F)c1] | 2-[1-(4-ethoxy-2,6- difluorobenzyl)- 1H-indazol-3-yl]- N-(pyridin-4-yl)- 6,7-dihydro-5H- pyrrolo[3,4- d]pyrimidin-4- amine, salt with hydrochloric acid | ¹H-NMR (400 MHz, DMSO-d₆): δ [ppm] = 1.29 (t, 3H), 4.05 (q, 2H), 4.65 (br. s., 2H), 4.79 (br. s., 2H), 5.77 (s, 2H), 6.75-6.97 (m, 2H), 7.35 (t, 1H), 7.56 (td, 1H), 7.92 (d, 1H), 8.47 (d, 1H), 8.56-8.73 (m, 4H), 10.00 (br. s., 2H), 11.30 (br. s., 1H), 14.17-15.00 (m, 1H). |
| 2-9-15 SM = 2-15-4 | 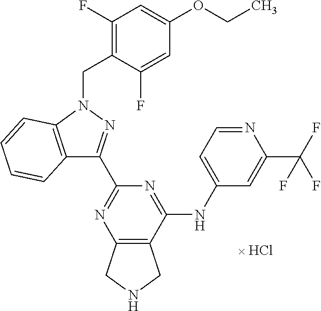 [SMILES: CCOc1cc(F)c(Cn2nc(-c3nc4c(c(Nc5ccnc(C(F)(F)F)c5)n3)CNC4)c3ccccc32)c(F)c1] | 2-[1-(4-ethoxy-2,6- difluorobenzyl)- 1H-indazol-3-yl]- N-[2-(trifluoro- methyl)pyridin-4- yl]-6,7-dihydro- 5H-pyrrolo[3,4- d]pyrimidin-4- amine, salt with hydrochloric acid | ¹H-NMR (400 MHz, DMSO-d₆): δ [ppm] = 1.28 (t, 3H), 4.04 (q, 2H), 4.53-4.65 (m, 2H), 4.69 (br. s., 2H), 5.75 (s, 2H), 6.81 (d, 2H), 7.30 (t, 1H), 7.43- 7.70 (m, 1H), 7.88 (d, 1H), 8.35-8.57 (m, 3H), 8.65 (d, 1H), 9.78 (br. s., 1H), 10.25 (s, 1H). |
| 2-9-16 SM = 2-15-5 | 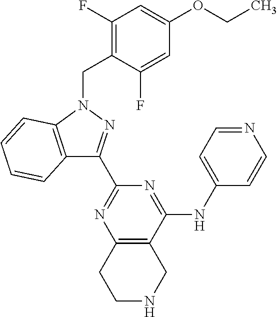 [SMILES: CCOc1cc(F)c(Cn2nc(-c3nc4c(c(Nc5ccncc5)n3)CNCC4)c3ccccc32)c(F)c1] | 2-[1-(4-ethoxy-2,6- difluorobenzyl)- 1H-indazol-3-yl]- N-(pyridin-4-yl)- 5,6,7,8-tetrahydro- pyrido[4,3-d]pyr- imidin-4-amine | ¹H-NMR (400 MHz, DMSO-d₆): δ [ppm] = 1.30 (t, 3H), 2.75- 2.88 (m, 2H), 3.01-3.13 (m, 2H), 3.89 (s, 2H), 4.05 (q, 2H), 5.71 (s, 2H), 6.73-6.88 (m, 2H), 7.26 (t, 1H), 7.49 (t, 1H), 7.85 (d, 1H), 8.06 (d, 2H), 8.41 (d, 2H), 8.49 (d, 1H), 8.69 (s, 1H). 1H not assigned. |
| 2-9-17 SM = 2-15-6 | 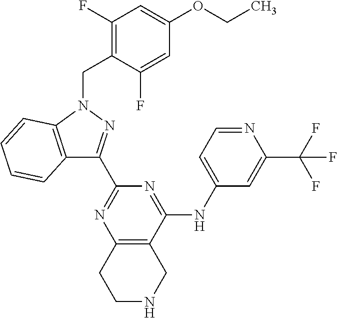 [SMILES: CCOc1cc(F)c(Cn2nc(-c3nc4c(c(Nc5ccnc(C(F)(F)F)c5)n3)CNCC4)c3ccccc32)c(F)c1] | 2-[1-(4-ethoxy-2,6- difluorobenzyl)- 1H-indazol-3-yl]- N-[2-(trifluorometh- yl)pyridin-4-yl]- 5,6,7,8-tetrahydro- pyrido[4,3-d]pyr- imidin-4-amine | ¹H-NMR (400 MHz, DMSO-d₆): δ [ppm] = 1.29 (t, 3H), 3.14 (t, 2H), 3.51 (t, 2H), 4.04 (q, 2H), 4.33 (s, 2H), 5.74 (s, 2H), 6.70- 6.87 (m, 2H), 7.28 (t, 1H), 7.44-7.58 (m, 1H), 7.85 (d, 1H), 8.41-8.56 (m, 3H), 8.63 (d, 1H), 9.29 (br. s., 1H), 9.50 (s, 1H). |
| 2-9-18 SM = 2-2-32 | 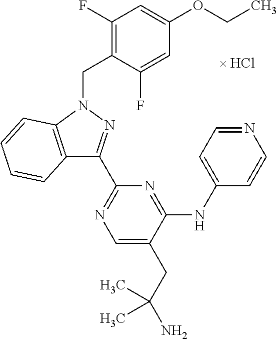 [SMILES: CCOc1cc(F)c(Cn2nc(-c3ncc(CC(C)(C)N)c(Nc4ccncc4)n3)c3ccccc32)c(F)c1] | 5-(2-amino-2- methylpropyl)-2- [1-(4-ethoxy-2,6- difluorobenzyl)- 1H-indazol-3-yl]- N-(pyridin-4- yl)pyrimidin-4- amine, salt with hydrochloric acid | ¹H-NMR (400 MHz, DMSO-d₆): δ [ppm] = 1.29 (t, 3H), 1.35 (s, 6H), 4.04 (q, 2H), 5.77 (s, 2H), 6.82 (d, 2H), 7.33 (t, 1H), 7.55 (dt, 1H), 7.91 (d, 1H), 8.15 (br.s, 3H), 8.46 (d, 1H), 8.63 (d, 2H), 8.74 (s, 1H), 8.82 (d, 2H), 10.61 (s, 1H), 14.5 (br.s, 1H), 2H's not assigned. |
| 2-9-19 SM = 2-2-43 | 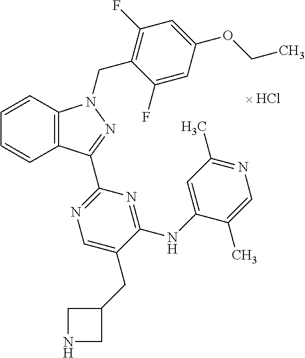 [SMILES: CCOc1cc(F)c(Cn2nc(-c3ncc(CC4CNC4)c(Nc4cc(C)ncc4C)n3)c3ccccc32)c(F)c1] | 5-(azetidin-3- ylmethyl)-N-(2,5- dimethylpyridin- 4-yl)-2-[1-(4- ethoxy-2,6- difluorobenzyl)- 1H-indazol-3- yl]pyrimidin-4- amine, salt with hydrochloric acid | ¹H-NMR (400 MHz, DMSO-d₆): δ [ppm] = 1.30 (t, 3H), 2.43 (br. s., 3H), 2.62 (s, 3H), 2.94- 3.08 (m, 2H), 3.13-3.30 (m, 1H), 3.64-3.90 (m, 3H), 4.04 (q, 2H), 5.75 (s, 2H), 6.74- 6.84 (m, 2H), 7.22-7.29 (m, 1H), 7.49-7.56 (m, 1H), 7.87 (d, 1H), 7.96 (s, 1H), 8.07- 8.19 (m, 1H), 8.25-8.42 (m, 3H), 8.58-8.78 (m, 2H). |
| 2-9-20 SM = 2-2-44 | 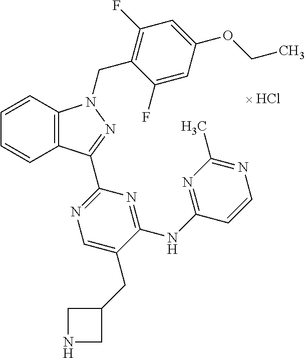 [SMILES: CCOc1cc(F)c(Cn2nc(-c3ncc(CC4CNC4)c(Nc4ccnc(C)n4)n3)c3ccccc32)c(F)c1] | 5-(azetidin-3- ylmethyl)-2-[1-(4- ethoxy-2,6- difluorobenzyl)- 1H-indazol-3-yl]- N-(2- methylpyrimidin- 4-yl)pyrimidin-4- amine, salt with hydrochloric acid | ¹H-NMR (400 MHz, DMSO-d₆): δ [ppm] = 1.29 (t, 3H), 2.73 (s, 3H), 2.79-2.88 (m, 1H), 2.91- 3.11 (m, 2H), 3.72-3.89 (m, 2H), 3.91-4.00 (m, 1H), 4.04 (q, 2H), 5.75 (s, 2H), 6.76- 6.85 (m, 2H), 7.27-7.35 (m, 1H), 7.50-7.57 (m, 1H), 7.88 (d, 1H), 8.17-8.28 (m, 2H), 8.31-8.39 (m, 1H), 8.41- 8.49 (m, 1H), 8.61 (d, 1H), 8.82 (s, 1H), 11.42 (br. s, 1H). |
| 2-9-21 SM = 2-2-45 | 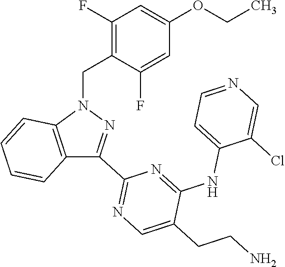 [SMILES: CCOc1cc(F)c(Cn2nc(-c3ncc(CCN)c(Nc4ccncc4Cl)n3)c3ccccc32)c(F)c1] | 5-(2-aminoethyl)-N- (3-chloropyridin-4- yl)-2-[1-(4-ethoxy- 2,6-difluorobenzyl)- 1H-indazol-3- yl]pyrimidin-4- amine | ¹H-NMR (400 MHz, DMSO-d₆): δ [ppm] = 1.29 (t, 3H), 2.74 (m, 2H), 2.95 (m, 2H), 4.04 (q, 2H), 5.69 (s, 2H), 5.95 (br. s, 2H), 6.79 (m, 2H), 7.20 (dd, 1H), 7.47 (dd, 1H), 7.83 (d, 1H), 8.32 (d, 1H), 8.34 (d, 1H), 8.47 (s, 1H), 8.51 (d, 1H), 8.54 (s, 1H). |
| 2-9-22 SM = 2-2-46 | 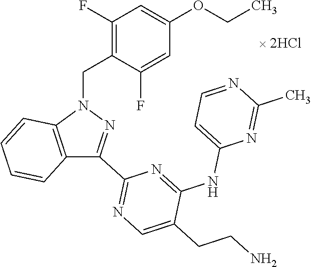 [SMILES: CCOc1cc(F)c(Cn2nc(-c3ncc(CCN)c(Nc4ccnc(C)n4)n3)c3ccccc32)c(F)c1] | 5-(2-aminoethyl)- 2-[1-(4-ethoxy-2,6- difluorobenzyl)-1H- indazol-3-yl]-N-(2- methylpyrimidin- 4-yl)pyrimidin-4- amine, salt with hydrochloric acid (1:2) | ¹H-NMR (400 MHz, DMSO-d₆): δ [ppm] = 1.29 (t, 3H), 2.70 (s, 3H), 3.13 (m, 4H), 4.03 (q, 2H), 5.74 (s, 2H), 6.79 (m, 2H), 7.31 (dd, 1H), 7.53 (dd, 1H), 7.87 (d, 1H), 8.08 (br. s., 3H), 8.28 (m, 1H), 8.45 (d, 1H), 8.61 (d, 1H), 8.81 (s, 1H), 11.39 (br. s., 1H). |
| 2-9-24 SM = 2-12-62 | 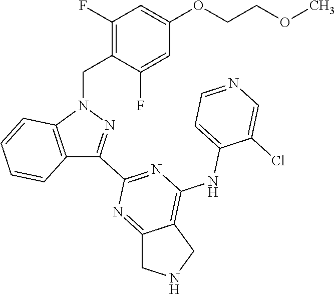 [SMILES: COCCOc1cc(F)c(Cn2nc(-c3nc4c(c(Nc5ccncc5Cl)n3)CNC4)c3ccccc32)c(F)c1] | N-(3-chloropyridin- 4-yl)-2-{1-[2,6- difluoro-4-(2-meth- oxyethoxy)benzyl]- 1H-indazol-3-yl}- 6,7-dihydro-5H- pyrrolo[3,4- d]pyrimidin-4- amine | ¹H-NMR (400 MHz, DMSO-d₆): δ [ppm] = 3.28 (s, 3H), 3.61 (m, 2H), 4.11 (m, 2H), 4.14 (br. s., 2H), 4.20 (br. s., 2H), 5.68 (s, 2H), 6.82 (m, 2H), 7.14 (dd, 1H), 7.45 (dd, 1H), 7.81 (d, 1H), 8.18 (d, 1H), 8.19 (d, 1H), 8.47 (d, 1H), 8.67 (s, 1H), 8.90 (br. s., 1H). |
| 2-9-25 SM = 2-12-63 | 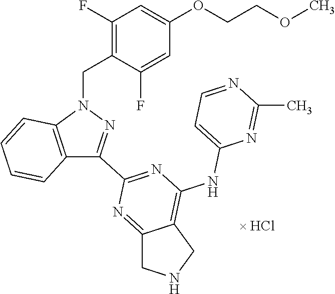 [SMILES: COCCOc1cc(F)c(Cn2nc(-c3nc4c(c(Nc5ccnc(C)n5)n3)CNC4)c3ccccc32)c(F)c1] | 2-{1-[2,6-difluoro- 4-(2-methoxy- ethoxy)benzyl]-1H- indazol-3-yl}-N-(2- methylpyrimidin- 4-yl)-6,7-dihydro- 5H-pyrrolo[3,4- d]pyrimidin-4- amine, salt with hydrochloric acid | ¹H-NMR (400 MHz, DMSO-d₆): δ [ppm] = 2.71 (s, 3H), 3.26 (s, 3H), 3.62 (m, 2H), 4.12 (m, 2H), 4.65 (br. s., 2H), 4.69 (br. s., 2H), 5.76 (s, 2H), 6.85 (m, 2H), 7.34 (dd, 1H), 7.55 (dd, 1H), 7.89 (d, 1H), 8.28 (m, 1H), 8.54 (d, 1H), 8.64 (d, 1H), 10.19 (m, 2H), 11.74 (br. s., 1H). |
| 2-9-26 SM = 2-12-64 | 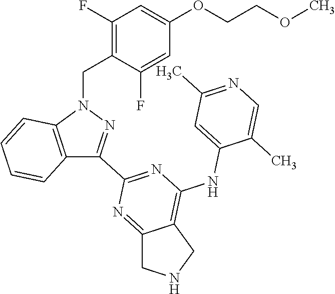 [SMILES: COCCOc1cc(F)c(Cn2nc(-c3nc4c(c(Nc5cc(C)ncc5C)n3)CNC4)c3ccccc32)c(F)c1] | 2-{1-[2,6-difluoro- 4-(2-methoxy- ethoxy)benzyl]-1H- indazol-3-yl}-N- (2,5- dimethylpyridin-4- yl)-6,7-dihydro-5H- pyrrolo[3,4-d]pyr- imidin-4-amine | ¹H-NMR (400 MHz, DMSO-d₆): δ [ppm] = 2.19 (s, 3H), 2.43 (s, 3H), 3.26 (s, 3H), 3.61 (m, 2H), 4.07 (br. s., 2H), 4.10 (br. s., 2H), 4.11 (m, 2H), 5.66 (s, 2H), 6.79 (m, 2H), 7.10 (dd, 1H), 7.43 (dd, 1H), 7.57 (s, 1H), 7.77 (d, 1H), 8.19 (d, 1H), 8.30 (s, 1H), 8.59 (br. s., 1H). |
| 2-9-27 SM = 2-12-97 | 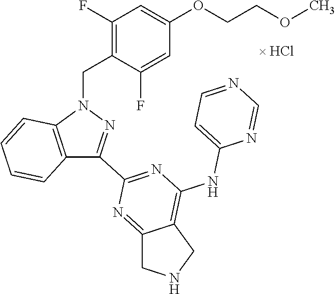 [SMILES: COCCOc1cc(F)c(Cn2nc(-c3nc4c(c(Nc5ccncn5)n3)CNC4)c3ccccc32)c(F)c1] | 2-{1-[2,6-difluoro- 4-(2-methoxy- ethoxy)benzyl]-1H- indazol-3-yl}-N- (pyrimidin-4-yl)- 6,7-dihydro-5H- pyrrolo[3,4-d]pyr- imidin-4-amine, salt with hydrochloric acid | ¹H-NMR (400 MHz, DMSO-d₆): δ [ppm] = 3.26 (s, 3H), 3.61 (m, 2H), 4.12 (m, 2H), 4.62 (br. s., 2H), 4.68 (br. s., 2H), 5.75 (s, 2H), 6.87 (m, 2H), 7.34 (dd, 1H), 7.54 (dd, 1H), 7.91 (d, 1H), 8.53 (d, 1H), 8.56 (d, 1H), 8.67 (d, 1H), 9.00 (s, 1H), 10.03 (m, 2H), 11.06 (br. s., 1H). |
| 2-9-28 SM = 2-12-98 | 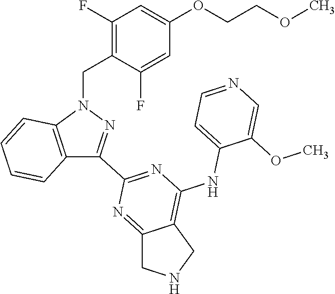 [SMILES: COCCOc1cc(F)c(Cn2nc(-c3nc4c(c(Nc5ccncc5OC)n3)CNC4)c3ccccc32)c(F)c1] | 2-{1-[2,6-difluoro- 4-(2-methoxy- ethoxy)benzyl]-1H- indazol-3-yl}-N-(3- methoxypyridin-4- yl)-6,7-dihydro-5H- pyrrolo[3,4-d]pyr- imidin-4-amine | ¹H-NMR (400 MHz, DMSO-d₆): δ [ppm] = 3.26 (s, 3H), 3.61 (m, 2H), 3.95 (s, 3H), 4.10 (br. s., 2H), 4.12 (m, 2H), 4.17 (br. s., 2H), 5.69 (s, 2H), 6.83 (m, 2H), 7.21 (dd, 1H), 7.47 (dd, 1H), 7.83 (d, 1H), 8.18 (d, 1H), 8.28 (br. s., 1H), 8.36 (d, 1H), 8.37 (s, 1H), 8.42 (d, 1H). |

### Example 2-10-1

### Preparation of 4-({2-[1-(4-ethoxy-2,6-difluorobenzyl)-1H-indazol-3-yl]-5-(3-methoxypropyl)pyrimidin-4-yl}amino)nicotinamide

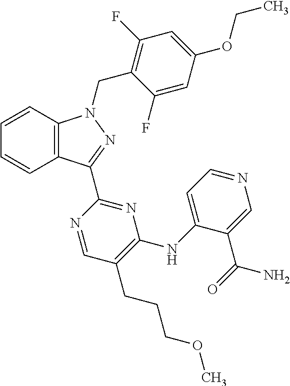 [SMILES: CCOc1cc(F)c(Cn2nc(-c3ncc(CCCOC)c(Nc4ccncc4C(N)=O)n3)c3ccccc32)c(F)c1]

68 mg 4-({2-[1-(4-ethoxy-2,6-difluorobenzyl)-1H-indazol-3-yl]-5-(3-methoxy-propyl)pyrimidin-4-yl}amino)nicotinic acid 2-2-10 (0.11 mmol, 1.0 eq.) was dissolved in 1.3 mL DMF, 0.13 mL ammonia solution (7N in methanol, 0.89 mmol, 7.5 eq), 68 mg benzotriazol-1-yl-oxytripyrrolidinophosphonium hexafluorophosphate (0.13 mmol, 1.1 eq.) and 0.08 mL N,N-Diisopropylethylamine (0.47 mmol, 4.0 eq.) were added. It was stirred at rt over night. The reaction mixture was diluted with water and a yellow precipitate was formed. It was filtered off, the filtrate was concentrated under reduced pressure. Both products were combined and purified by flash chromatography to provide 42 mg of the target compound in 85% purity: 53%

¹H-NMR (300 MHz, DMSO-d₆): δ [ppm]=1.28 (t, 3H), 1.84-1.95 (m, 2H), 2.64-2.74 (m, 2H), 3.24 (s, 3H), 3.41 (t, 2H), 4.03 (q, 2H), 5.64-5.80 (m, 2H), 6.74-6.87 (m, 2H), 7.29 (s, 1H), 7.45-7.56 (m, 1H), 7.88 (d, 1H), 8.02 (s, 1H), 8.43-8.59 (m, 4H), 8.98 (s, 1H), 9.33 (d, 1H), 12.30 (s, 1H).

The following compound was prepared according to the same procedure from the indicated starting materials (SM=starting material):

| | | | |
|---|---|---|---|
| 2-10-2 SM = 1-15-1 30.0 eq. ammonia, 1.3 eq. PYBOP, 5.0 eq. DIPEA | 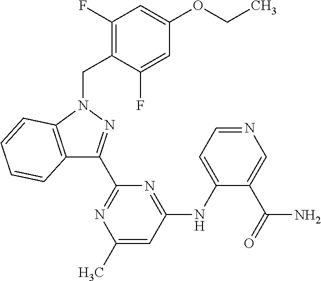 [SMILES: CCOc1cc(F)c(Cn2nc(-c3nc(C)cc(Nc4ccncc4C(N)=O)n3)c3ccccc32)c(F)c1] | 4-({2-[1-(4-ethoxy- 2,6-difluorobenzyl)- 1H-indazol-3-yl]-6- methylpyrimidin-4- yl}amino)pyridine- 3-carboxamide | ¹H-NMR (400 MHz, DMSO-d₆): δ [ppm] = 1.30 (t, 3H), 4.05 (q, 2H), 5.73 (s, 2H), 6.82 (d, 2H), 6.88 (s, 1H), 7.31 (t, 1H), 7.52 (t, 1H), 7.88 (d, 1H), 7.93 (br. s., 1H), 8.49 (d, 2H), 8.55 (d, 1H), 8.93 (s, 1H), 9.06 (d, 1H), 11.81 (s, 1H). 3H not assigned |

### Example 2-11-1

### Preparation of 2-[1-(4-ethoxy-2,6-difluorobenzyl)-1H-indazol-3-yl]-N-(pyridin-4-yl)-5-{[1-(2,2,2-trifluoroethyl)azetidin-3-yl]methyl}pyrimidin-4-amine

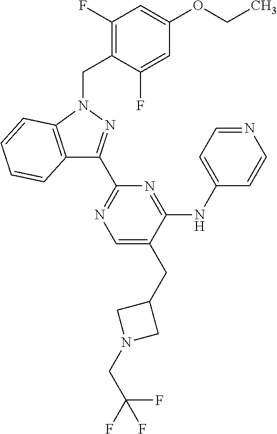 [SMILES: CCOc1cc(F)c(Cn2nc(-c3ncc(CC4CN(CC(F)(F)F)C4)c(Nc4ccncc4)n3)c3ccccc32)c(F)c1]

80 mg 5-(azetidin-3-ylmethyl)-2-[1-(4-ethoxy-2,6-difluorobenzyl)-1H-indazol-3-yl]-N-(pyridin-4-yl)pyrimidin-4-amine dihydrochloride 2-9-4 (0.11 mmol, 1.0 eq.), 86 mg potassium carbonate (0.61 mmol, 4.6 eq.) and 2 mg potassium iodide (0.01 mmol, 0.1 eq.) were suspended in 1.5 mL acetonitrile. 19 μL 2,2,2-trifluoroethyl trifluoromethanesulfonate (0.13 mmol, 1.0 eq.) were added and it was stirred at rt for 48 hours. The reaction mixture was filtered through a HPLC filter. The filtrate was concentrated under reduced pressure and purified by flash chromatography to provide 33 mg of the target compound in 99% purity: 40%.

¹H-NMR (500 MHz, DMSO-d₆): δ [ppm]=1.28 (t, 3H), 2.78-2.91 (m, 1H), 3.00 (d, 2H), 3.11 (t, 2H), 3.14-3.21 (m, 2H), 3.51 (t, 2H), 4.03 (q, 2H), 5.69 (s, 2H), 6.73-6.83 (m, 2H), 7.24 (t, 1H), 7.49 (ddd, 1H), 7.86 (d, 1H), 8.03-8.12 (m, 2H), 8.37 (s, 1H), 8.39-8.47 (m, 3H), 8.89 (s, 1H).

The following compounds were prepared according to the same procedure from the indicated starting materials (SM=starting material):

| | | | |
|---|---|---|---|
| 2-11-2 SM = 2-9-1 | 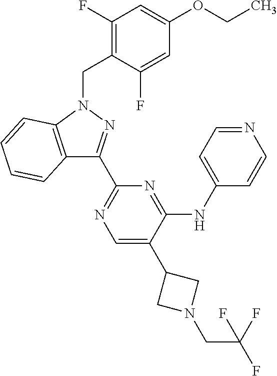 [SMILES: CCOc1cc(F)c(Cn2nc(-c3ncc(C4CN(CC(F)(F)F)C4)c(Nc4ccncc4)n3)c3ccccc32)c(F)c1] | 2-[1-(4-ethoxy-2,6- difluorobenzyl)- 1H-indazol-3-yl]- N-(pyridin-4- yl)-5-[1-(2,2,2- trifluoroeth- yl)azetidin-3- yl]pyrimidin-4- amine | ¹H-NMR (400 MHz, DMSO-d₆): δ [ppm] = 1.30 (t, 3H), 3.41 (q, 2H), 3.53 (br. s., 2H), 3.96 (s, 3H), 4.06 (q, 2H), 5.68-5.80 (m, 2H), 6.70-6.88 (m, 2H), 7.29 (t, 1H), 7.46-7.57 (m, 1H), 7.89 (d, 1H), 8.06 (d, 2H), 8.41-8.51 (m, 3H), 8.55 (s, 1H), 9.82 (s, 1H). |
| 2-11-3 SM: 2-9-1 Side product of 2-11-2 | 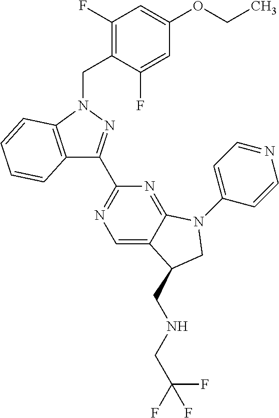 [SMILES: CCOc1cc(F)c(Cn2nc(-c3ncc4c(n3)N(c3ccncc3)C[C@H]4CNCC(F)(F)F)c3ccccc32)c(F)c1] | N-({2-[1-(4- ethoxy-2,6- difluorobenzyl)-1H- indazol-3-yl]-7- (pyridin-4-yl)-6,7- dihydro-5H- pyrrolo[2,3d]pyr- imidin-5- yl}methyl)-2,2,2- trifluoroethanamine | ¹H-NMR (400 MHz, DMSO-d₆): δ [ppm] = 1.31 (t, 3H), 2.74-2.86 (m, 1H), 2.88-3.03 (m, 2H), 3.37 (q, 2H), 3.61- 3.74 (m, 1H), 3.97 (dd, 1H),4.06 (q, 2H), 4.25 (t, 1H), 5.72 (s, 2H), 6.77-6.87 (m, 2H), 7.33 (t, 1H), 7.52 (t, 1H), 7.87 (d, 1H), 7.98-8.10 (m, 2H), 8.47-8.57 (m, 4H). |
| 2-11-4 SM: 2-9-7 | 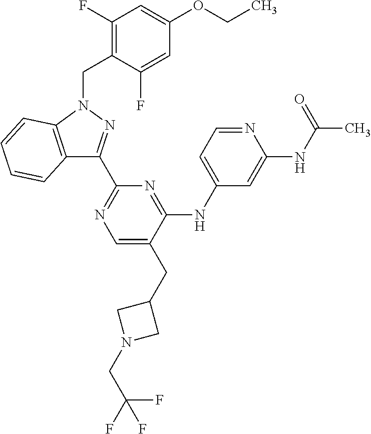 [SMILES: CCOc1cc(F)c(Cn2nc(-c3ncc(CC4CN(CC(F)(F)F)C4)c(Nc4ccnc(NC(C)=O)c4)n3)c3ccccc32)c(F)c1] | N-{4-[(2-[1-(4- ethoxy-2,6- difluorobenzyl)-1H- indazol-3-yl]-5-{[1- (2,2,2-trifluoroeth- yl)azetidin-3- yl]methyl}pyrimidin- 4-yl)amino]pyr- idin-2-yl}acetamide | ¹H-NMR (400 MHz, DMSO-d₆): δ [ppm] = 1.30 (t, 3 H) 1.98-2.15 (m, 3 H) 2.25 (m, 1 H) 2.55-2.79 (m, 3 H) 2.85- 3.03 (m, 1 H) 3.19-3.30 (m, 2 H) 3.67 (dd, 1 H) 3.95 (d, 1 H) 4.04 (q, 2 H) 5.63 (s, 2 H) 6.74 (s, 1 H) 6.76 (s, 1 H) 7.00 (t, 1 H) 7.31 (dd, 1 H) 7.36-7.47 (m, 1 H) 7.74 (d, 1 H) 8.02 (d, 1 H) 8.20-8.28 (m, 1 H) 8.31 (t, 2 H) 10.60 (s, 1 H). |
| 2-11-5 SM: 2-17-5 1.5 eq. 2,2- Difluoroethyl- trifluoro- methane- sulfonate | 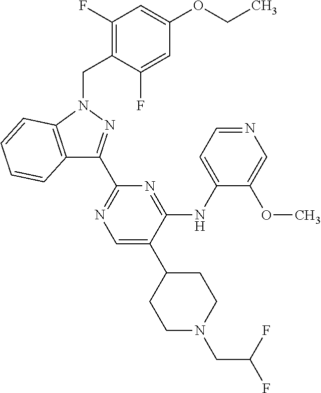 [SMILES: CCOc1cc(F)c(Cn2nc(-c3ncc(C4CCN(CC(F)F)CC4)c(Nc4ccncc4OC)n3)c3ccccc32)c(F)c1] | 5-[1-(2,2- difluoroethyl)piper- idin-4-yl]-2-[1-(4- ethoxy-2,6- difluorobenzyl)-1H- indazol-3-yl]-N-(3- methoxypyridin-4- yl)pyrimidin-4-amine | ¹H-NMR (400 MHz, DMSO-d₆): δ [ppm] = 1.29 (t, 3H), 1.72-1.85 (m, 2H), 1.86-1.94 (m, 2H), 2.36-2.46 (m, 2H), 2.72-2.90 (m, 3H), 3.07- 3.15 (m, 2H), 3.99 (s, 3H), 4.04 (q, 2H), 5.70 (s, 2H), 6.20 (tt, 1H), 6.76-6.86 (m, 2H), 7.19- 7.27 (m, 1H), 7.45-7.52 (m, 1H), 7.86 (d, 1H), 8.13 (s, 1H), 8.21 (d, 1H), 8.34 (d, 1H), 8.39 (s, 1H), 8.52 (s, 1H), 8.62 (d, 1H). |

### Example 2-12-1

### Preparation of 2-[1-(4-ethoxy-2,6-difluorobenzyl)-1H-indazol-3-yl]-6-methyl-N-(pyridin-4-yl)pyrimidin-4-amine

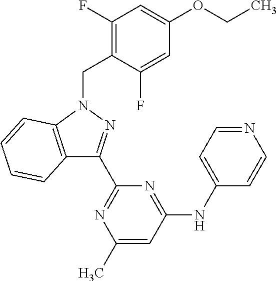 [SMILES: CCOc1cc(F)c(Cn2nc(-c3nc(C)cc(Nc4ccncc4)n3)c3ccccc32)c(F)c1]

64 mg of 3-(4-chloro-6-methylpyrimidin-2-yl)-1-(4-ethoxy-2,6-difluorobenzyl)-1H-indazole 1-13-2 (0.15 mmol, 1.0 eq.) were dissolved in 2 mL dioxane, and 17 mg pyridin-4-amine (0.18 mmol, 1.2 eq.), 3 mg palladium(II)acetate (0.01 mmol, 0.1 eq), 13 mg (9,9-dimethyl-9H-xanthene-4,5-diyl)bis(diphenylphosphine) (0.02 mmol, 0.15 eq) and 150 mg cesium carbonate (0.46 mmol, 3.0 eq.) were added. The reaction mixture was stirred at 105° C. under argon atmosphere in a sealed vessel over night. All unsolved compounds were filtered off and washed with dichloromethane/2-propanole. The filtrate was concentrated under reduced pressure and purified by flash chromatography to provide 19 mg of the target compound in 92% purity: 26%.

1H-NMR (400 MHz, DMSO-d₆): d [ppm]=1.29 (t, 3H), 2.49 (s, 3H), 4.05 (q, 2H), 5.72 (s, 2H), 6.32-6.50 (m, 1H), 6.70 (s, 1H), 6.76-6.88 (m, 2H), 7.30 (t, 1H), 7.51 (td, 1H), 7.89-7.93 (m, 2H), 8.34-8.47 (m, 2H), 8.56 (d, 1H), 9.99 (s, 1H).

The following compounds were prepared according to the same procedure from the indicated starting materials (SM=starting material):

| | | | |
|---|---|---|---|
| 2-12-2 SM = 1-13-1 | 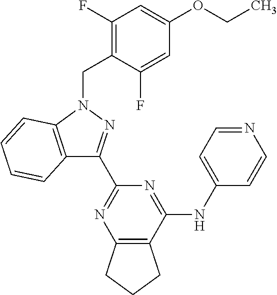 [SMILES: CCOc1cc(F)c(Cn2nc(-c3nc4c(c(Nc5ccncc5)n3)CCC4)c3ccccc32)c(F)c1] | 2-[1-(4-ethoxy-2,6- difluorobenzyl)-1H- indazol-3-yl]-N-(pyridin- 4-yl)-6,7-dihydro-5H- cyclopenta[d]pyrimidin- 4-amine | ¹H-NMR (400 MHz, DMSO-d₆): δ [ppm] = 1.30 (t, 3H), 2.13 (quin, 2H), 2.89-3.06 (m, 4H), 4.04 (q, 2H), 5.71 (s, 2H), 6.72-6.88 (m, 2H), 7.23- 7.31 (m, 1H), 7.50 (ddd, 1H), 7.86 (d, 1H), 8.01- 8.13 (m, 2H), 8.36-8.46 (m, 2H), 8.51 (d, 1H), 9.19 (s, 1H). |
| 2-12-3 SM = 1-13-3 | 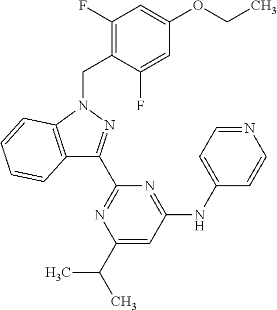 [SMILES: CCOc1cc(F)c(Cn2nc(-c3nc(Nc4ccncc4)cc(C(C)C)n3)c3ccccc32)c(F)c1] | 2-[1-(4-ethoxy-2,6- difluorobenzyl)-1H- indazol-3-yl]-6-(propan- 2-yl)-N-(pyridin-4- yl)pyrimidin-4-amine | ¹H-NMR (300 MHz, DMSO-d₆): δ [ppm] = 1.23-1.39 (m, 9H), 2.90- 3.07 (m, 1H), 4.03 (q, 2H), 5.71 (s, 2H), 6.69 (s, 1H), 6.80 (d, 2H), 7.26-7.39 (m, 1H), 7.50 (t, 1H), 7.87 (d, 1H), 7.93 (d, 2H), 8.38 (d, 2H), 8.56 (d, 1H), 10.00 (s, 1H). |
| 2-12-4 SM = 1-13-4 | 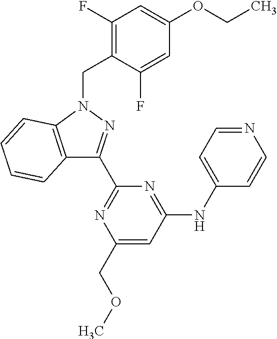 [SMILES: CCOc1cc(F)c(Cn2nc(-c3nc(COC)cc(Nc4ccncc4)n3)c3ccccc32)c(F)c1] | 2-[1-(4-ethoxy-2,6- difluorobenzyl)-1H- indazol-3-yl]-6- (methoxymethyl)-N- (pyridin-4-yl)pyrimidin- 4-amine | ¹H-NMR (400 MHz, DMSO-d₆): δ [ppm] = 1.30 (t, 3H), 3.48 (s, 3H), 4.05 (q, 2H), 4.56 (s, 2H), 5.73 (s, 2H), 6.76-6.85 (m, 2H), 6.90 (s, 1H), 7.23-7.34 (m, 1H), 7.52 (ddd, 1H), 7.88 (d, 1H), 7.91-7.95 (m, 2H), 8.35- 8.46 (m, 2H), 8.52 (d, 1H), 10.12 (s, 1H). |
| 2-12-5 SM = 1-13-5 | 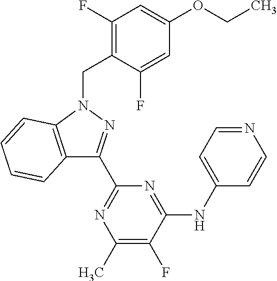 [SMILES: CCOc1cc(F)c(Cn2nc(-c3nc(C)c(F)c(Nc4ccncc4)n3)c3ccccc32)c(F)c1] | 2-[1-(4-ethoxy-2,6- difluorobenzyl)-1H- indazol-3-yl]-5-fluoro- 6-methyl-N-(pyridin-4- yl)pyrimidin-4-amine | ¹H-NMR (400 MHz, DMSO-d₆): δ [ppm] = 1.30 (t, 3H), 2,55 (s, 3H), 4.05 (q, 2H), 5.71 (s, 2H), 6.72-6.87 (m, 2H), 7.30 (t, ¹H), 7.51 (ddd, 1H), 7.87 (d, 1H), 8.02-8.12 (m, 2H), 8.38-8.45 (m, 2H), 8.49 (d, 1H), 9.94 (br. s., 1H). |
| 2-12-6 SM = 1-13-1 | 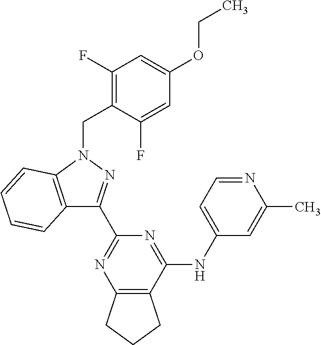 [SMILES: CCOc1cc(F)c(Cn2nc(-c3nc4c(c(Nc5ccnc(C)c5)n3)CCC4)c3ccccc32)c(F)c1] | 2-[1-(4-ethoxy-2,6- difluorobenzyl)-1H- indazol-3-yl]-N-(2- methylpyridin-4-yl)-6,7- dihydro-5H- cyclopenta[d]pyrimidin- 4-amine | ¹H-NMR (400 MHz, DMSO-d₆): δ [ppm] = 1.29 (t, 3H), 2.12 (quin, 2H), 2.44 (s, 3H), 2.86- 3.04 (m, 4H), 4.05 (q, 2H), 5.71 (s, 2H), 6.71- 6.82 (m, 2H), 7.27 (t, 1H), 7.50 (ddd, 1H), 7.80 (dd, 1H), 7.84 (d, 1H), 7.98 (d, 1H), 8.28 (d, 1H), 8.52 (d, 1H), 9.08 (s, 1H). |
| 2-12-8 SM = 1-13-6 | 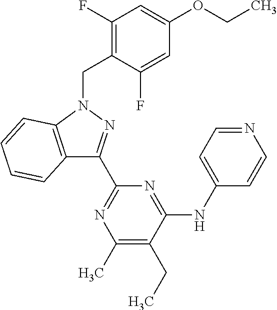 [SMILES: CCOc1cc(F)c(Cn2nc(-c3nc(C)c(CC)c(Nc4ccncc4)n3)c3ccccc32)c(F)c1] | 2-[1-(4-ethoxy-2,6- difluorobenzyl)-1H- indazol-3-yl]-5-ethyl-6- methyl-N-(pyridin-4- yl)pyrimidin-4-amine | ¹H-NMR (400 MHz, DMSO-d₆): δ [ppm] = 1.12 (t, 3H), 1.26 (t, 3H), 2.52 (s, 3H), 2.81 (q, 2H), 4.03 (q, 2H), 5.69 (s, 2H), 6.72-6.82 (m, 2H), 7.24 (t, 1H), 7.39-7.52 (m, 1H), 7.83 (d, 1H), 8.08 (d, 2H), 8.38 (d, 2H), 8.49 (d, 1H), 8.75 (s, 1H). |
| 2-12-9 SM = 1-13-7 | 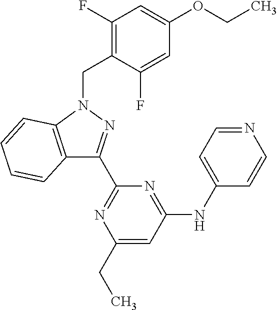 [SMILES: CCOc1cc(F)c(Cn2nc(-c3nc(CC)cc(Nc4ccncc4)n3)c3ccccc32)c(F)c1] | 2-[1-(4-ethoxy-2,6- difluorobenzyl)-1H- indazol-3-yl]-6-ethyl-N- (pyridin-4-yl)pyrimidin- 4-amine | ¹H-NMR (400 MHz, DMSO-d₆): δ [ppm] = 1.23-1.36 (m, 6H), 2.71- 2.82 (m, 2H), 4.03 (q, 2H), 5.69 (s, 2H), 6.69 (s, 1H), 6.76-6.84 (m, 2H), 7.30 (t, 1H), 7.50 (ddd, 1H), 7.85 (d, 1H), 7.89- 7.97 (m, 2H), 8.38 (d, 2H), 8.55 (d, 1H), 9.98 (s, 1H). |
| 2-12-12 SM = 1-13-8 | 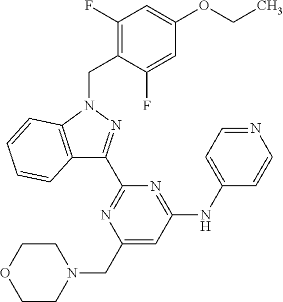 [SMILES: CCOc1cc(F)c(Cn2nc(-c3nc(CN4CCOCC4)cc(Nc4ccncc4)n3)c3ccccc32)c(F)c1] | 2-[1-(4-ethoxy-2,6- difluorobenzyl)-1H- indazol-3-yl]-6- (morpholin-4-ylmethyl)- N-(pyridin-4- yl)pyrimidin-4-amine | ¹H-NMR (400 MHz, DMSO-d₆): δ [ppm] = 1.28 (t, 3 H) 3.66 (br. s., 6 H (4H + 2H)) 4.03 (q, 2 H) 5.71 (s, 2 H) 6.80 (d, 2 H) 6.96 (s, 1 H) 7.29 (t, 1 H) 7.50 (t, 1 H) 7.86 (d, 1 H) 7.92 (d, 2 H) 8.39 (d, 2 H) 8.53 (d, 1 H) 10.07 (s, 1 H); 4H not assigned, hidden in DMSO-signal |
| 2-12-13 SM = 1-13-8 | 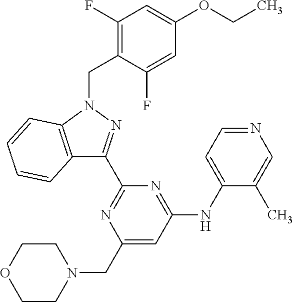 [SMILES: CCOc1cc(F)c(Cn2nc(-c3nc(CN4CCOCC4)cc(Nc4ccncc4C)n3)c3ccccc32)c(F)c1] | 2-[1-(4-ethoxy-2,6- difluorobenzyl)-1H- indazol-3-yl]-N-(3- methylpyridin-4-yl)-6- (morpholin-4- ylmethyl)pyrimidin-4- amine | ¹H NMR (400 MHz, DMSO-d₆) δ ppm 1.26 (t, 3 H) 2.30 (s, 3 H) 2.51-2.57 (m, 4 H) 3.59-3.70 (m, 4 H) 3.74 (s, 2 H) 4.03 (q, 2 H) 5.58 (s, 1 H) 5.69 (s, 1 H) 6.74-6.80 (m, 2 H) 7.13-7.25 (m, 2 H) 7.41-7.49 (m, 1 H) 7.79 (dd, 1 H) 8.28-8.36 (m, 3 H) 8.44 (t, 1 H) 9.00 (s, 1 H). |
| 2-12-14 SM = 1-13-8 | 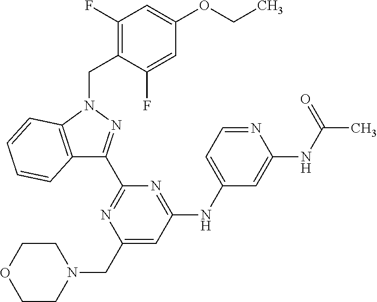 [SMILES: CCOc1cc(F)c(Cn2nc(-c3nc(CN4CCOCC4)cc(Nc4ccnc(NC(C)=O)c4)n3)c3ccccc32)c(F)c1] | N-[4-({2-[1-(4-ethoxy- 2,6-difluorobenzyl)-1H- indazol-3-yl]-6- (morpholin-4- ylmethyl)pyrimidin-4- yl}amino)pyridin-2- yl]acetamide | ¹H NMR (400 MHz, DMSO-d₆) δ ppm 1.28 (t, 3 H) 2.09 (s, 3 H) 2.51-2.60 (m, 4 H) 3.56-3.75 (m, 6 H) 4.03 (q, 2 H) 5.70 (s, 2 H) 6.77 (s, 1 H) 6.80 (s, 1 H) 7.00 (s, 1 H) 7.27 (t, 1 H) 7.42-7.57 (m, 1 H) 7.83 (d, 1 H) 7.94-8.05 (m, 1 H) 8.13 (d, 1 H) 8.37 (dd, 1 H) 8.53 (d, 1 H) 10.14 (s, 1 H) 10.33 (s, 1 H). |
| 2-12-15 SM = 1-13-9 | 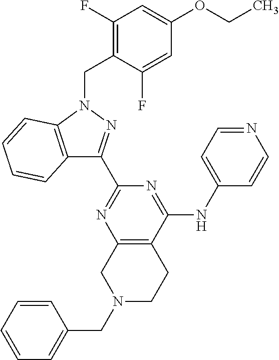 [SMILES: CCOc1cc(F)c(Cn2nc(-c3nc4c(c(Nc5ccncc5)n3)CCN(Cc3ccccc3)C4)c3ccccc32)c(F)c1] | 7-benzyl-2-[1-(4- ethoxy-2,6- difluorobenzyl)-1H- indazol-3-yl]-N- (pyridin-4-yl)-5,6,7,8- tetrahydropyrido[3,4- d]pyrimidin-4-amine | ¹H-NMR (400 MHz, DMSO-d₆): δ [ppm] = 1.29 (t, 3H),2.82 (dd, 4H), 3.56-3.67 (m, 2H), 3.76 (s, 2H), 4.04 (q, 2H), 5.70 (s, 2H), 6.69- 6.88 (m, 2H), 7.21-7.27 (m, 1H), 7.28-7.33 (m, 1H), 7.35-7.45 (m, 4H), 7.48 (ddd, 1H), 7.84 (d, 1H), 7.98-8.15 (m, 2H), 8.35-8.47 (m, 3H), 8.84 (s, 1H). |
| 2-12-16 SM = 1-13-9 DMF instead dioxane | 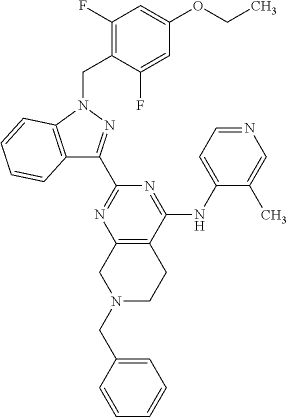 [SMILES: CCOc1cc(F)c(Cn2nc(-c3nc4c(c(Nc5ccncc5C)n3)CCN(Cc3ccccc3)C4)c3ccccc32)c(F)c1] | 7-benzyl-2-[1-(4- ethoxy-2,6- difluorobenzyl)-1H- indazol-3-yl]-N-(3- methylpyridin-4-yl)- 5,6,7,8- tetrahydropyrido[3,4- d]pyrimidin-4-amine | ¹H-NMR (400 MHz, DMSO-d₆): δ [ppm] = 1.28 (t, 3H), 2.21 (s, 3H), 2.73-2.89 (m, 4H), 3.56 (s, 2H), 3.74 (s, 2H), 4.03 (q, 2H), 5.62 (s, 2H), 6.65-6.81 (m, 2H), 7.05 (t, 1H), 7.24- 7.32 (m, 1H), 7.32-7.44 (m, 5H), 7.73 (d, 1H), 7.85 (d, 1H), 8.03 (d, 1H), 8.14 (s, 1H), 8.37 (d, 1H), 8.42 (s, 1H). |
| 2-12-17 SM = 1-13-10 DMF instead dioxane | 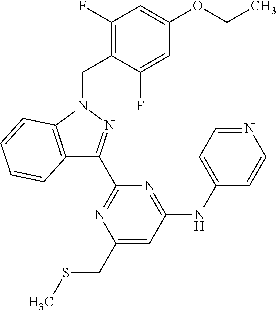 [SMILES: CCOc1cc(F)c(Cn2nc(-c3nc(CSC)cc(Nc4ccncc4)n3)c3ccccc32)c(F)c1] | 2-[1-(4-ethoxy-2,6- difluorobenzyl)-1H- indazol-3-yl]-6- [(methylsulfanyl)meth- yl]-N-(pyridin-4- yl)pyrimidin-4-amine | ¹H-NMR (400 MHz, DMSO-d₆): δ [ppm] = 1.29 (t, 3H), 3.83 (s, 2H), 4.05 (q, 2H), 5.73 (s, 2H), 6.82 (d, 2H), 6.87 (s, 1H), 7.31 (t, 1H), 7.52 (m, 1H), 7.88 (d, 2H), 8.41 (d, 2H), 8.58 (d, 1H), 10.12 (s, 1H), 4H's not assigned. |
| 2-12-18 SM = 1-13-1 | 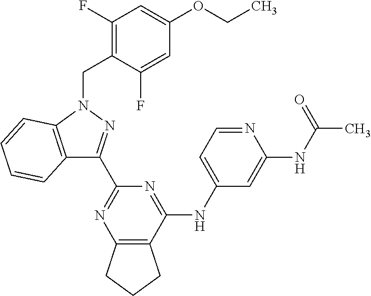 [SMILES: CCOc1cc(F)c(Cn2nc(-c3nc4c(c(Nc5ccnc(NC(C)=O)c5)n3)CCC4)c3ccccc32)c(F)c1] | N-[4-({2-[1-(4-ethoxy- 2,6-difluorobenzyl)-1H- indazol-3-yl]-6,7- dihydro-5H- cyclopenta[d]pyrimidin- 4-yl}amino)pyridin-2- yl]acetamide | ¹H NMR (400 MHz, DMSO-d₆) δ ppm 1.28 (t, 3 H) 1.96-2.18 (m, 5 H) 2.85-3.07 (m, 4 H) 4.03 (q, 2 H) 5.68 (s, 2 H) 6.69-6.83 (m, 2 H) 7.22 (t, 1 H) 7.47 (td, 1 H) 7.81 (d, 1 H) 8.11 (d, 1 H) 8.18 (s, 1 H) 8.34 (dd, 1 H) 8.48 (d, 1 H) 9.35 (s, 1 H) 10.29 (s, 1 H). |
| 2-12-19 SM = 1-13-4 DMF instead dioxane | 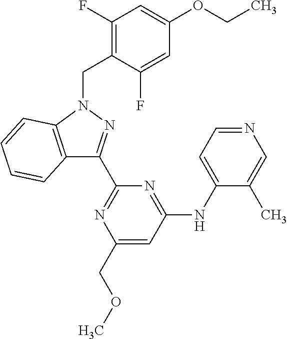 [SMILES: CCOc1cc(F)c(Cn2nc(-c3nc(COC)cc(Nc4ccncc4C)n3)c3ccccc32)c(F)c1] | 2-[1-(4-ethoxy-2,6- difluorobenzyl)-1H- indazol-3-yl]-6- (methoxymethyl)-N-(3- methylpyridin-4- yl)pyrimidin-4-amine | LC-MS (Methode with NH₃): retention time: 1.36 min MS ES+: 517.2 [M + H]⁺ |
| 2-12-20 SM = 1-13-4 DMF instead dioxane | 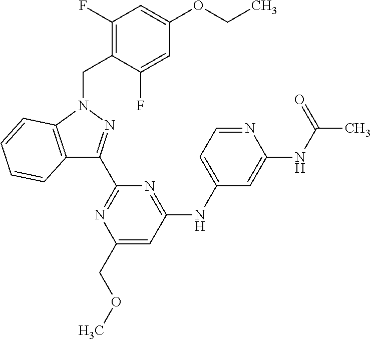 [SMILES: CCOc1cc(F)c(Cn2nc(-c3nc(COC)cc(Nc4ccnc(NC(C)=O)c4)n3)c3ccccc32)c(F)c1] | N-[4-({2-[1-(4-ethoxy- 2,6-difluorobenzyl)-1H- indazol-3-yl]-6- (methoxymethyl)pyr- imidin-4- yl}amino)pyridin- 2-yl]acetamide | ¹H-NMR (400 MHz, DMSO-d₆): δ [ppm] = 1.28 (t, 3H), 2.09 (s, 3H), 3.45 (s, 3H), 4.03 (q, 2H), 4.53 (s, 2H), 5.70 (s, 2H), 6.63-6.83 (m, 2H), 6.90 (s, 1H), 7.17-7.32 (m, 1H), 7.43-7.55 (m, 1H), 7.80 (d, 1H), 7.95-8.07 (m, 1H), 8.14 (d, 1H), 8.32-8.41 (m, 1H), 8.49 (d, 1H), 10.18 (s, 1H), 10.34 (s, 1H). |
| 2-12-21 SM = 1-13-5 | 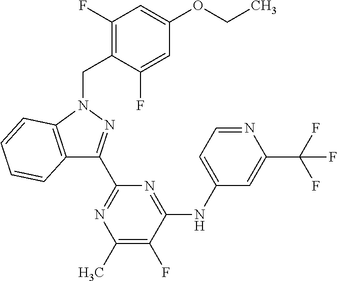 [SMILES: CCOc1cc(F)c(Cn2nc(-c3nc(C)c(F)c(Nc4ccnc(C(F)(F)F)c4)n3)c3ccccc32)c(F)c1] | 2-[1-(4-ethoxy-2,6- difluorobenzyl)-1H- indazol-3-yl]-5-fluoro- 6-methyl-N-[2- (trifluoromethyl)pyridin- 4-yl]pyrimidin-4-amine | ¹H-NMR (400 MHz, DMSO-d₆): δ [ppm] = 1.29 (t, 3H), 2.53-2.55 (m, 3H), 4.04 (q, 2H), 5.72 (s, 2H), 6.68-6.89 (m, 2H), 7.28 (t, 1H), 7.51 (t, 1H), 7.84 (d, 1H), 8.37 (br. s., 1H), 8.47 (d, 1H), 8.50-8.64 (m, 2H), 10.34 (br. s., 1H). |
| 2-12-22 SM = 1-13-7 DMF instead dioxane | 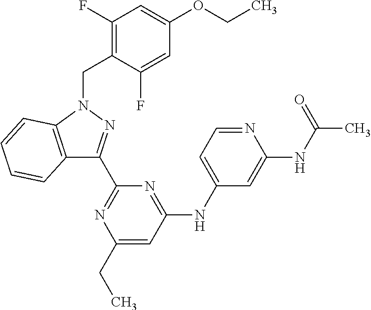 [SMILES: CCOc1cc(F)c(Cn2nc(-c3nc(CC)cc(Nc4ccnc(NC(C)=O)c4)n3)c3ccccc32)c(F)c1] | N-[4-({2-[1-(4-ethoxy- 2,6-difluorobenzyl)-1H- indazol-3-yl]-6- ethylpyrimidin-4- yl}amino)pyridin-2- yl]acetamide | ¹H-NMR (400 MHz, DMSO-d₆): δ [ppm] = 1.25-1.38 (m, 6H), 2.11 (s, 3H), 2.76 (d, 2H), 4.05 (q, 2H), 5.72 (s, 2H), 6.69-6.88 (m, 3H), 7.26- 7.33 (m, 1H), 7.46-7.55 (m, 1H), 7.84 (d, 1H), 8.00 (s, 1H), 8.14 (d, 1H), 8.38-8.44 (m, 1H), 8.57 (d, 1H), 10.11 (s, 1H), 10.36 (s, 1H). |
| 2-12-23 SM = 1-13-11 | 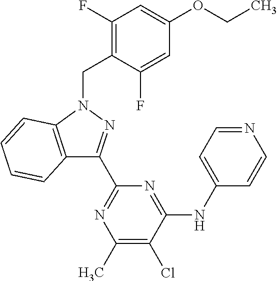 [SMILES: CCOc1cc(F)c(Cn2nc(-c3nc(C)c(Cl)c(Nc4ccncc4)n3)c3ccccc32)c(F)c1] | 5-chloro-2-[1-(4-ethoxy- 2,6-difluorobenzyl)-1H- indazol-3-yl]-6-methyl- N-(pyridin-4- yl)pyrimidin-4-amine | ¹H-NMR (400 MHz, DMSO-d₆): δ [ppm] = 1.30 (t, 3H), 2.63 (s, 3H), 4.05 (q, 2H), 5.73 (s, 2H), 6.74-6.87 (m, 2H), 7.23- 7.33 (m, 1H), 7.52 (ddd, 1H), 7.88 (d, 1H), 8.04- 8.17 (m, 2H), 8.40-8.52 (m, 3H), 9.34 (s, 1H). |
| 2-12-24 SM = 1-13-12 | 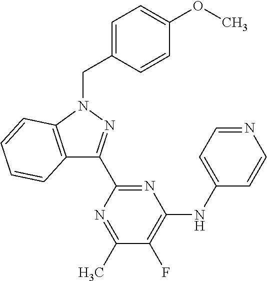 [SMILES: COc1ccc(Cn2nc(-c3nc(C)c(F)c(Nc4ccncc4)n3)c3ccccc32)cc1] | 5-fluoro-2-[1-(4- methoxybenzyl)-1H- indazol-3-yl]-6-methyl- N-(pyridin-4-yl)- pyrimidin-4-amine | ¹H-NMR (300 MHz, DMSO-d₆): δ [ppm] = 2.51 (d, 3H), 3.68 (s, 3H), 5.69 (s, 2H), 6.79- 6.98 (m, 2H), 7.18-7.37 (m, 3H), 7.43 (t, 1H), 7.81 (d, 1H), 8.03 (d, 2H), 8.44 (d, 3H), 9.98 (s, 1H). |
| 2-12-26 SM = 1-13-1 | 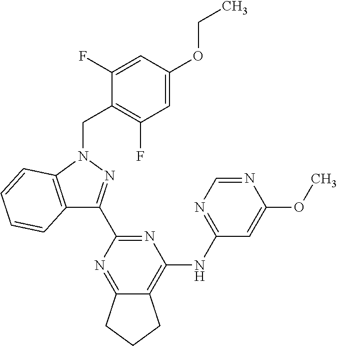 [SMILES: CCOc1cc(F)c(Cn2nc(-c3nc4c(c(Nc5cc(OC)ncn5)n3)CCC4)c3ccccc32)c(F)c1] | 2-[1-(4-ethoxy-2,6- difluorobenzyl)-1H- indazol-3-yl]-N-(6- methoxypyrimidin-4- yl)-6,7-dihydro-5H- cyclopenta[d]pyrimidin- 4-amine | ¹H-NMR (400 MHz, DMSO-d₆): δ [ppm] = 1.29 (t, 3 H) 2.09 (quin, 2 H) 2.98-3.02 (m, 4 H) 3.96 (s, 3 H) 4.04 (q, 2 H) 5.71 (s, 2 H) 6.75 (s, 1 H) 6.77 (s, 1 H) 7.19-7.33 (m, 1 H) 7.50 (ddd, 1 H) 7.82 (d, 1 H) 8.02 (d, 1 H) 8.44-8.60 (m, 2 H) 9.85 (s, 1 H). |
| 2-12-27 SM = 1-13-1 | 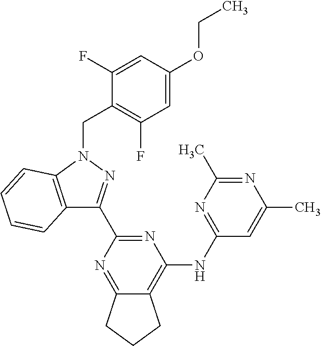 [SMILES: CCOc1cc(F)c(Cn2nc(-c3nc4c(c(Nc5cc(C)nc(C)n5)n3)CCC4)c3ccccc32)c(F)c1] | N-(2,6- dimethylpyrimidin-4- yl)-2-[1-(4-ethoxy-2,6- difluorobenzyl)-1H- indazol-3-yl]-6,7- dihydro-5H- cyclopenta[d]pyrimidin- 4-amine | ¹H-NMR (300 MHz, DMSO-d₆): δ [ppm] = 1.29 (t, 3 H) 2.06-2.10 (m, 2 H) 2.39 (s, 3 H) 2.98- 3.01 (m, 4 H) 3.33 (s, 3 H) 4.04 (q, 2 H) 5.73 (s, 2 H) 6.75 (s, 1 H) 6.78 (s, 1 H) 7.29 (d, 1 H) 7.50 (s, 1 H) 7.84 (d, 1 H) 8.31 (s, 1 H) 8.54 (d, 1 H) 9.78 (s, 1 H). |
| 2-12-28 SM = 1-13-1 | 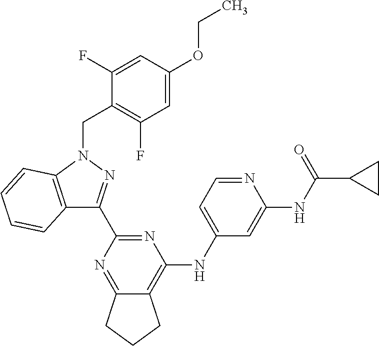 [SMILES: CCOc1cc(F)c(Cn2nc(-c3nc4c(c(Nc5ccnc(NC(=O)C6CC6)c5)n3)CCC4)c3ccccc32)c(F)c1] | N-[4-({2-[1-(4-ethoxy- 2,6-difluorobenzyl)-1H- indazol-3-yl]-6,7- dihydro-5H- cyclopenta[d]pyrimidin- 4-yl}amino)pyridin-2- yl]cyclopropanecarbox- amide | ¹H-NMR (400 MHz, DMSO-d₆): δ [ppm] = 0.62-0.88 (m, 4H) 1.30 (t, 3 H) 1.92-2.19 (m, 3 H) 2.87-3.05 (m, 4H) 4.05 (q, 2 H) 5.70 (s, 2 H) 6.78 (s, 1 H) 6.81 (s, 1 H) 7.24 (t, 1 H) 7.41-7.59 (m, 1 H) 7.83 (d, 1 H) 8.14 (d, 1 H) 8.22 (d, 1 H) 8.36 (dd, 1 H) 8.49 (d, 1 H) 9.33 (s, 1 H) 10.63 (s, 1 H) |
| 2-12-30 SM = 1-25-1 | 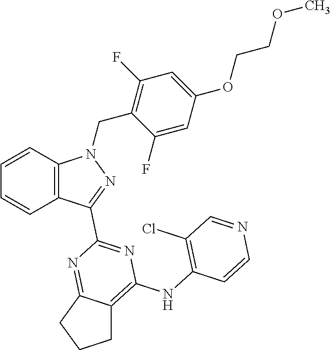 [SMILES: COCCOc1cc(F)c(Cn2nc(-c3nc4c(c(Nc5ccncc5Cl)n3)CCC4)c3ccccc32)c(F)c1] | N-(3-chloropyridin-4-yl)- 2-{1-[2,6-difluoro-4-(2- methoxyethoxy)benzyl]- 1H-indazol-3-yl}-6,7- dihydro-5H- cyclopenta[d]pyrimidin- 4-amine | ¹H-NMR (300 MHz, DMSO-d₆): δ [ppm] = 2.04-2.21 (m, 2 H) 2.84- 3.06 (m, 4 H) 3.27 (s, 3 H) 3.62 (dd, 2 H) 4.12 (dd, 2 H) 5.69 (s, 2 H) 6.82 (s, 1 H) 6.84 (s, 1 H) 7.18 (t, 1 H) 7.34-7.43 (m, 1 H) 7.43-7.54 (m, 1 H) 7.82 (d, 1 H) 8.27 (d, 1 H) 8.35-8.50 (m, 2 H) 8.64 (s, 1 H). |
| 2-12-31 SM = 1-25-1 | 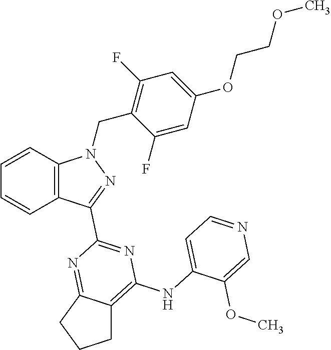 [SMILES: COCCOc1cc(F)c(Cn2nc(-c3nc4c(c(Nc5ccncc5OC)n3)CCC4)c3ccccc32)c(F)c1] | 2-{1-[2,6-difluoro-4-(2- methoxyethoxy)benzyl]- 1H-indazol-3-yl}-N-(3- methoxypyridin-4-yl)- 6,7-dihydro-5H- cyclopenta[d]pyrimidin- 4-amine | ¹H-NMR (400 MHz, DMSO-d₆): δ [ppm] = 2.03-2.19 (m, 2 H) 2.86- 3.05 (m, 4 H) 3.24-3.28 (s, 3 H) 3.59-3.67 (m, 2 H) 3.98 (s, 3 H) 4.13 (dd, 2 H) 5.70 (s, 2 H) 6.83 (s, 1 H) 6.85 (s, 1 H) 7.24 (t, 1 H) 7.49 (ddd, 1 H) 7.84 (d, 1 H) 7.92 (s, 1 H) 8.18 (d, 1 H) 8.36 (s, 1 H) 8.43 (d, 1 H) 8.62 (d, 1 H). |
| 2-12-32 SM = 1-13-1 | 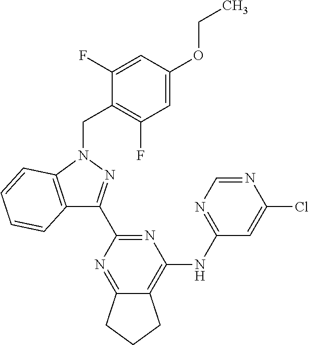 [SMILES: CCOc1cc(F)c(Cn2nc(-c3nc4c(c(Nc5cc(Cl)ncn5)n3)CCC4)c3ccccc32)c(F)c1] | N-(6-chloropyrimidin-4- yl)-2-[1-(4-ethoxy-2,6- difluorobenzyl)-1H- indazol-3-yl]-6,7- dihydro-5H- cyclopenta[d]pyrimidin- 4-amine | ¹H-NMR (400 MHz, DMSO-d₆): δ [ppm] = 1.28 (t, 3 H) 2.09 (quin, 2 H) 3.01 (t, 4 H) 4.02 (q, 2 H) 5.71 (s, 2 H) 6.73 (s, 1 H) 6.75 (s, 1 H) 7.28 (t, 1 H) 7.49 (td, 1 H) 7.82 (d, 1 H) 8.51 (d, 1 H) 8.70 (dd, 2 H) 10.38 (s, 1 H). |
| 2-12-33 SM = 1-13-8 | 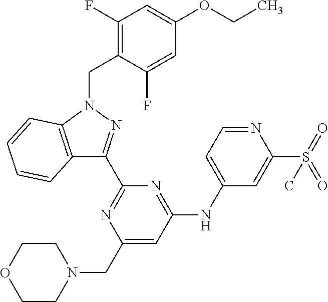 [SMILES: CCOc1cc(F)c(Cn2nc(-c3nc(CN4CCOCC4)cc(Nc4ccnc(S(C)(=O)=O)c4)n3)c3ccccc32)c(F)c1] | 2-[1-(4-ethoxy-2,6- difluorobenzyl)-1H- indazol-3-yl]-N-[2- (methylsulfonyl)pyridin- 4-yl]-6-(morpholin-4- ylmethyl)pyrimidin-4- amine | ¹H-NMR (400 MHz, DMSO-d₆): δ [ppm] = 1.27 (m, 3 H) 2.51-2.63 (m, 4 H) 3.26-3.32 (m, 2 H) 3.60-3.78 (m, 7 H) 4.02 (q, 2 H) 5.72 (s, 2 H) 6.78 (s, 1 H) 6.80 (s, 1 H) 7.01 (s, 1 H) 7.14-7.37 (m, 1 H) 7.37-7.53 (m, 1 H) 7.81 (d, 1 H) 8.12 (d, 1 H) 8.51 (d, 1 H) 8.56 (d, 1 H) 8.61-8.79 (m, 1 H) 10.56 (s, 1 H). |
| 2-12-34 SM = 1-13-8 | 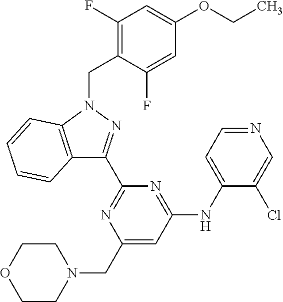 [SMILES: CCOc1cc(F)c(Cn2nc(-c3nc(CN4CCOCC4)cc(Nc4ccncc4Cl)n3)c3ccccc32)c(F)c1] | N-(3-chloropyridin-4-yl)- 2-[1-(4-ethoxy-2,6- difluorobenzyl)-1H- indazol-3-yl]-6- (morpholin-4- ylmethyl)pyrimidin-4- amine | ¹H-NMR (400 MHz, DMSO-d₆): δ [ppm] = 1.28 (t, 3 H) 2.51-2.58 (m, 4 H) 3.58-3.82 (m, 6 H) 4.03 (q, 2 H) 5.70 (s, 2 H) 6.77 (s, 1 H) 6.79 (s, 1 H) 6.94 (d, 1 H) 6.91 (d, 1 H) 7.16 (td, 1 H) 7.26 (t, 1 H) 8.36 (d, 1 H) 8.46 (d, 1 H) 8.57 (s, 1 H) 8.81 (d, 1 H) 9.30 s, 1 H). |
| 2-12-35 SM = 1-13-8 | 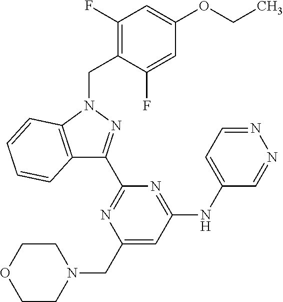 [SMILES: CCOc1cc(F)c(Cn2nc(-c3nc(CN4CCOCC4)cc(Nc4ccnnc4)n3)c3ccccc32)c(F)c1] | N-{2-[1-(4-ethoxy-2,6- difluorobenzyl)-1H- indazol-3-yl]-6- (morpholin-4- ylmethyl)pyrimidin-4- yl}pyridazin-4-amine | ¹H-NMR (400 MHz, DMSO-d₆): δ [ppm] = 1.28 (t, 3 H) 2.52-2.55 (m, 4 H) 3.55-3.75 (m, 6 H) 4.04 (q, 2 H) 5.71 (s, 2 H) 6.80 (s, 1 H) 6.82 (s, 1 H) 7.02 (s, 1 H) 7.30 (t, 1 H) 7.45-7.57 (m, 1 H) 7.86 (d, 1 H) 8.52 (d, 1 H) 8.55-8.64 (m, 1 H) 8.92 (dd, 1 H) 9.34 (dd, 1 H) 10.40 (br. s., 1 H). |
| 2-12-36 SM = 1-25-2 + 1-22-2 | 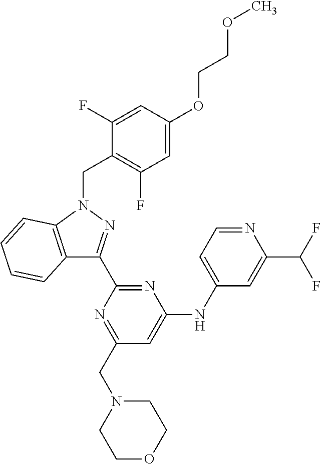 [SMILES: COCCOc1cc(F)c(Cn2nc(-c3nc(CN4CCOCC4)cc(Nc4ccnc(C(F)F)c4)n3)c3ccccc32)c(F)c1] | 2-{1-[2,6-difluoro-4-(2- methoxyethoxy)benzyl]- 1H-indazol-3-yl}-N-[2- (difluoromethyl)pyridin- 4-yl]-6-(morpholin-4- ylmethyl)pyrimidin-4- amine | ¹H-NMR (400 MHz, DMSO-d₆): δ [ppm] = 3.26 (s, 3 H) 3.38-3.97 (m, 10 H) 4.12 (dd, 2 H) 4.47 (br. s., 2 H) 5.76 (s, 2 H) 6.78-7.05 (m, 4 H) 7.33 (t, 1 H) 7.54 (t, 1 H) 7.88 (d, 1 H) 8.06-8.18 (m, 1 H) 8.24 (d, 1 H) 8.45- 8.66 (m, 2 H) 10.67 (s, 1 H). |
| 2-12-37 SM = 1-25-2 | 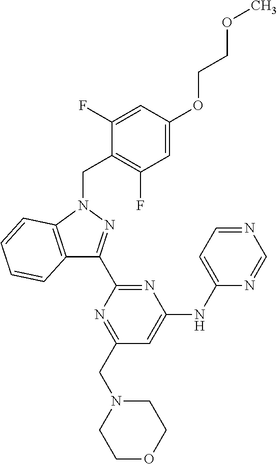 [SMILES: COCCOc1cc(F)c(Cn2nc(-c3nc(CN4CCOCC4)cc(Nc4ccncn4)n3)c3ccccc32)c(F)c1] | 2-{1-[2,6-difluoro-4-(2- methoxyethoxy)benzyl]- 1H-indazol-3-yl}-6- (morpholin-4-ylmethyl)- N-(pyrimidin-4- yl)pyrimidin-4-amine | ¹H-NMR (400 MHz, DMSO-d₆): δ [ppm] = 3.27 (s, 3 H) 3.33-54 (m, 4 H) 3.62 (dd, 2 H) 3.87 (br. s., 4 H) 4.12 (dd, 2 H) 4.53 (br. s., 2 H) 5.75 (s, 2 H) 6.84 (s, 1 H) 6.87 (s, 1 H) 7.36 (t, 1 H) 7.55 (t, 1 H) 7.79-7.95 (m, 2 H) 7.99 (br. s., 1 H) 8.64 (d, 2 H) 8.93 (br. s., 1 H) 11.02 (s, 1 H). |
| 2-12-38 SM = 1-13-8 | 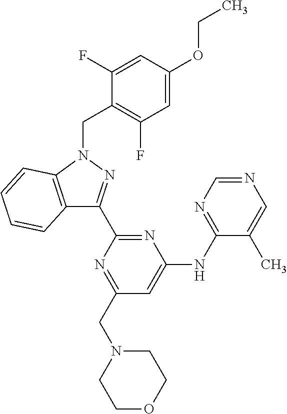 [SMILES: CCOc1cc(F)c(Cn2nc(-c3nc(CN4CCOCC4)cc(Nc4ncncc4C)n3)c3ccccc32)c(F)c1] | 2-[1-(4-ethoxy-2,6- difluorobenzyl)-1H- indazol-3-yl]-N-(5- methylpyrimidin-4-yl)- 6-(morpholin-4- ylmethyl)pyrimidin-4- amine | ¹H-NMR (400 MHz, DMSO-d₆): δ [ppm] = 1.28 (t, 3 H) 2.33 (s, 3 H) 2.51-2.55 (m, 4 H) 3.59- 3.76 (m, 6 H) 4.03 (q, 2 H) 5.70 (s, 2 H) 6.77 (s, 1 H) 6.75 (s, 1 H) 7.28 (t, 1 H) 7.42-7.52 (m, 1 H) 7.77 (d, 1 H) 8.09 (s, 1 H) 8.43 (s, 1 H) 8.67 (d, 1 H) 8.74 (s, 1 H) 9.46 (s, 1 H). |
| 2-12-39 SM = 1-25-2 | 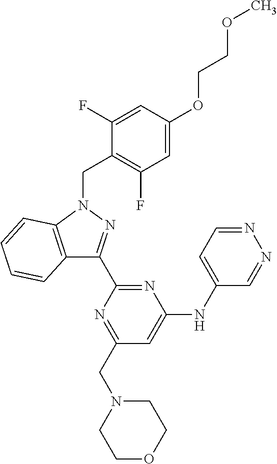 [SMILES: COCCOc1cc(F)c(Cn2nc(-c3nc(CN4CCOCC4)cc(Nc4ccnnc4)n3)c3ccccc32)c(F)c1] | N-[2-{1-[2,6-difluoro-4- (2- methoxyethoxy)benzyl]- 1H-indazol-3-yl}-6- (morpholin-4- ylmethyl)pyrimidin-4- yl]pyridazin-4-amine | ¹H-NMR (400 MHz, DMSO-d₆): δ [ppm] = 2.51-2.59 (m, 4 H) 3.18- 3.28 (m, 3 H) 3.54-3.76 (m, 8 H) 4.01-4.17 (m, 2 H) 5.72 (s, 2 H) 6.84 (s, 1 H) 6.86 (s, 1 H) 7.02 (s, 1 H) 7.30 (t, 1 H) 7.50 (ddd, 1 H) 7.86 (d, 1 H) 8.51 (d, 1 H) 8.59 (dd, 1 H) 8.94 (d, 1 H) 9.30- 9.40 (m, 1 H) 10.35 (s, 1 H). |
| 2-12-40 SM = 1-25-2 | 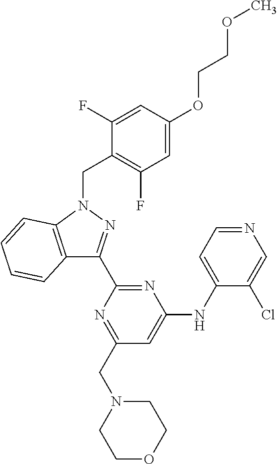 [SMILES: COCCOc1cc(F)c(Cn2nc(-c3nc(CN4CCOCC4)cc(Nc4ccncc4Cl)n3)c3ccccc32)c(F)c1] | N-(3-chloropyridin-4-yl)- 2-{1-[2,6-difluoro-4-(2- methoxyethoxy)benzyl]- 1H-indazol-3-yl}-6- (morpholin-4- ylmethyl)pyrimidin-4- amine | ¹H-NMR (400 MHz, DMSO-d₆): δ [ppm] = 2.51-2.62 (m, 4 H) 3.25 (s, 3 H) 3.54-3.74 (m, 8 H) 4.02-4.20 (m, 2 H) 5.70 (s, 2 H) 6.81 (s, 1 H) 6.83 (s, 1 H) 7.26 (t, 1 H) 7.32- 7.41 (m, 2 H) 7.41- 7.53 (m, 1 H) 7.84 (d, 1 H) 8.37 (d, 1 H) 8.46 (d, 1 H) 8.57 (s, 1 H) 8.81 (d, 1 H) 9.38 (br. s., 1 H). |
| 2-12-41 SM = 1-25-2 | 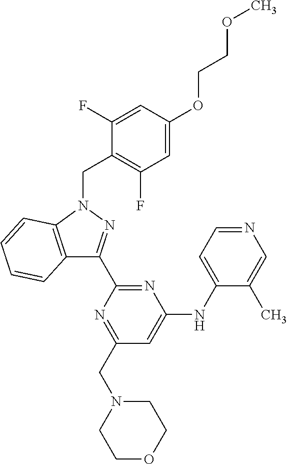 [SMILES: COCCOc1cc(F)c(Cn2nc(-c3nc(CN4CCOCC4)cc(Nc4ccncc4C)n3)c3ccccc32)c(F)c1] | 2-{1-[2,6-difluoro-4-(2- methoxyethoxy)benzyl]- 1H-indazol-3-yl}-N-(3- methylpyridin-4-yl)-6- (morpholin-4- ylmethyl)pyrimidin-4- amine | ¹H-NMR (400 MHz, DMSO-d₆): δ [ppm] = 2.30 (s, 3 H) 2.51-2.56 (m, 4 H) 3.25 (s, 3 H) 3.53- 3.72 (m, 8 H) 4.04-4.17 (m, 2 H) 5.69 (s, 2 H) 6.79 (s, 1 H) 6.82 (s, 1 H) 7.15 (s, 1 H) 7.22 (t, 1 H) 7.47 (ddd, 1 H) 7.81 (d, 1 H) 8.24-8.38 (m, 3 H) 8.45 (d, 1 H) 9.01 (s, 1 H). |
| 2-12-42 SM = 1-25-2 | 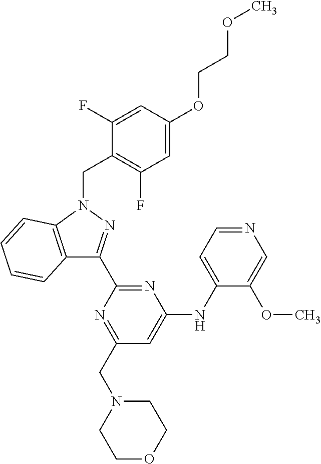 [SMILES: COCCOc1cc(F)c(Cn2nc(-c3nc(CN4CCOCC4)cc(Nc4ccncc4OC)n3)c3ccccc32)c(F)c1] | 2-{1-[2,6-difluoro-4-(2- methoxyethoxy)benzyl]- 1H-indazol-3-yl}-N-(3- methoxypyridin-4-yl)-6- (morpholin-4- ylmethyl)pyrimidin-4- amine | ¹H-NMR (400 MHz, DMSO-d₆): δ [ppm] = 2.51-2.60 (m, 4 H) 3.20- 3.28 (m, 3 H) 3.54-3.70 (m, 8 H) 3.98 (s, 2 H) 4.04-4.16 (m, 2 H) 5.70 (s, 2 H) 6.81 (s, 1 H) 6.83 (s, 1 H) 7.27 (t, 1 H) 7.32 (s, 1 H) 7.49 (ddd, 1 H) 7.84 (d, 1 H) 8.13 (d, 1 H) 8.30 (s, 1 H) 8.51 (d, 1 H) 8.76-8.93 (m, 1 H) 9.26 (s, 1 H). |
| 2-12-43 SM = 1-25-2 | 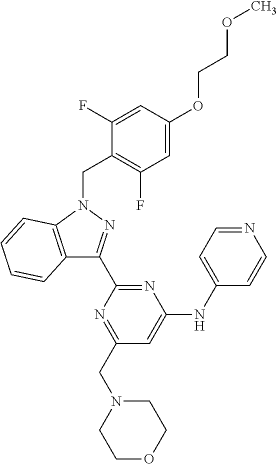 [SMILES: COCCOc1cc(F)c(Cn2nc(-c3nc(CN4CCOCC4)cc(Nc4ccncc4)n3)c3ccccc32)c(F)c1] | 2-{1-[2,6-difluoro-4-(2- methoxyethoxy)benzyl]- 1H-indazol-3-yl}-6- (morpholin-4-ylmethyl)- N-(pyridin-4- yl)pyrimidin-4-amine | ¹H-NMR (400 MHz, DMSO-d₆): δ [ppm] = 2.51-2.61 (m, 4 H) 3.25 (s, 3 H) 3.55-3.75 (m, 8 H) 4.03-4.23 (m, 2 H) 5.71 (s, 2 H) 6.82 (s, 1 H) 6.84 (s, 1 H) 6.96 (s, 1 H) 7.29 (t, 1 H) 7.50 (ddd, 1 H) 7.85 (d, 1 H) 7.88-8.00 (m, 2 H) 8.34-8.44 (m, 2 H) 8.53 (d, 1 H) 10.05 (s, 1 H). |
| 2-12-44 SM = 1-13-8 | 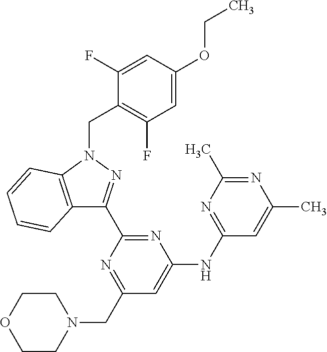 [SMILES: CCOc1cc(F)c(Cn2nc(-c3nc(CN4CCOCC4)cc(Nc4cc(C)nc(C)n4)n3)c3ccccc32)c(F)c1] | N-(2,6- dimethylpyrimidin-4-yl)- 2-[1-(4-ethoxy-2,6- difluorobenzyl)-1H- indazol-3-yl]-6- (morpholin-4- ylmethyl)pyrimidin-4- amine | ¹H-NMR (300 MHz, DMSO-d₆): δ [ppm] = 1.27 (t, 3 H) 2.37 (s, 3 H) 2.49-2.53 (m, 7 H) 3.59- 3.79 (m, 6 H) 4.02 (q, 2 H) 5.72 (s, 2 H) 6.74 (s, 1 H) 6.77 (s, 1 H) 7.23- 7.42 (m, 2 H) 7.42-7.64 (m, 1 H) 7.64-7.93 (m, 2 H) 8.61 (d, 1 H) 10.54 (s, 1 H). |
| 2-12-45 SM = 1-13-8 | 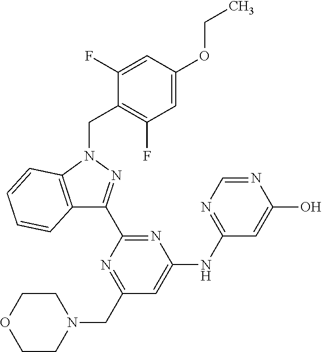 [SMILES: CCOc1cc(F)c(Cn2nc(-c3nc(CN4CCOCC4)cc(Nc4cc(O)ncn4)n3)c3ccccc32)c(F)c1] | 6-({2-[1-(4-ethoxy-2,6- difluorobenzyl)-1H- indazol-3-yl]-6- (morpholin-4- ylmethyl)pyrimidin-4- yl}amino)pyrimidin-4-ol | ¹H-NMR (300 MHz, DMSO-d₆): δ [ppm] = 1.27 (t, 3 H) 2.51-2.61 (m, 4 H) 3.50-3.83 (m, 6 H) 4.01 (q, 2 H) 5.70 (s, 2 H) 6.73 (s, 1 H) 6.77 (s, 1 H) 6.97 (s, 1 H) 7.26 (t, 1 H) 7.36-7.64 (m, 2 H) 7.77 (d, 1 H) 8.11 (s, 1 H) 8.55 (d, 1 H) 10.18 (s, 1 H) 12.13 (br. s., 1 H). |
| 2-12-46 SM = 1-13-8 | 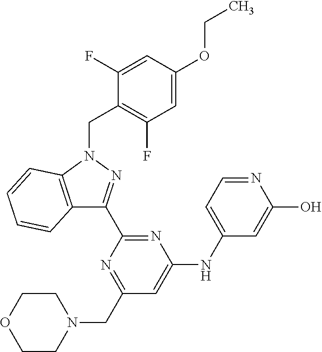 [SMILES: CCOc1cc(F)c(Cn2nc(-c3nc(CN4CCOCC4)cc(Nc4ccnc(O)c4)n3)c3ccccc32)c(F)c1] | 4-({2-[1-(4-ethoxy-2,6- difluorobenzyl)-1H- indazol-3-yl]-6- (morpholin-4- ylmethyl)pyrimidin-4- yl}amino)pyridin-2-ol | ¹H-NMR (300 MHz, DMSO-d₆): δ [ppm] = 1.28 (t, 3 H) 2.51-2.63 (m, 4 H) 3.57-3.65 (m, 4 H) 3.76 (s, 2 H) 4.02 (q, 2 H) 5.31 (d, 1 H) 5.74 (s, 2 H) 6.02 (dd, 1 H) 6.60 (s, 2 H) 6.74 (s, 1 H) 6.78 (s, 1 H) 7.32 (t, 1 H) 7.43- 7.56 (m, 1 H) 7.80 (d, 1 H) 7.91-8.10 (m, 2 H) 8.49 (d, 1 H). |
| 2-12-47 SM = 1-13-8 | 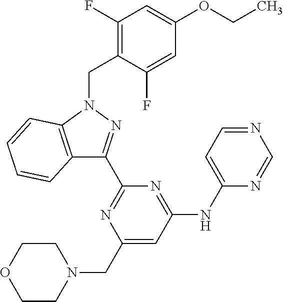 [SMILES: CCOc1cc(F)c(Cn2nc(-c3nc(CN4CCOCC4)cc(Nc4ccncn4)n3)c3ccccc32)c(F)c1] | 2-[1-(4-ethoxy-2,6- difluorobenzyl)-1H- indazol-3-yl]-6- (morpholin-4-ylmethyl)- N-(pyrimidin-4- yl)pyrimidin-4-amine | ¹H-NMR (300 MHz, DMSO-d₆): δ [ppm] = 1.27 (t, 3 H) 2.49-2.54 (m, 4 H) 3.51-3.83 (m, 6 H) 4.02 (q, 2 H) 5.71 (s, 2 H) 6.78 (s, 1 H) 6.81 (s, 1 H) 7.06-7.40 (m, 1 H) 7.50 (t, 1 H) 7.66-7.92 (m, 2 H) 8.12 (d, 1 H) 8.36- 8.68 (m, 2 H) 8.84 (s, 1 H) 10.70 (s, 1 H). |
| 2-12-48 SM = 1-13-4 | 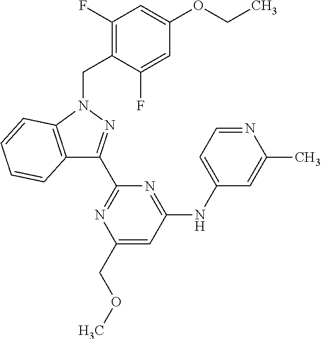 [SMILES: CCOc1cc(F)c(Cn2nc(-c3nc(COC)cc(Nc4ccnc(C)c4)n3)c3ccccc32)c(F)c1] | 2-[1-(4-ethoxy-2,6- difluorobenzyl)-1H- indazol-3-yl]-6- (methoxymethyl)-N-(2- methylpyridin-4- yl)pyrimidin-4-amine | ¹H-NMR (300 MHz, DMSO-d₆): δ [ppm] = 1.28 (t, 3H), 2.44 (s, 3H), 3.46 (s, 3H), 4.02 (q, 2H), 4.54 (s, 2H), 5.72 (s, 2H), 6.69-6.83 (m, 2H), 6.87 (s, 1H), 7.22-7.35 (m, 1H), 7.44-7.55 (m, 1H), 7.60 (dd, 1H), 7.79-7.94 (m, 2H), 8.27 (d, 1H), 8.52 (d, 1H), 10.01 (s, 1H). |
| 2-12-49 SM = 1-25-3 TBS protection group was removed during the reaction | 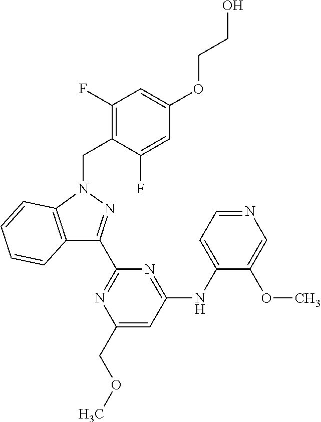 [SMILES: COCc1cc(Nc2ccncc2OC)nc(-c2nn(Cc3c(F)cc(OCCO)cc3F)c3ccccc23)n1] | 2-{3,5-difluoro-4-[(3-{4- (methoxymethyl)-6-[(3- methoxypyridin-4- yl)amino]pyrimidin-2- yl}-1H-indazol-1- yl)methyl]phenoxy}eth- anol | ¹H-NMR (400 MHz, DMSO-d₆): δ [ppm] = 3.47 (s, 3H), 3.61-3.73 (m, 2H), 3.93-4.08 (m, 5H), 4.55 (s, 2H), 4.90 (t, 1H), 5.72 (s, 2H), 6.75-6.90 (m, 2H), 7.23-7.34 (m, 2H), 7.51 (t, 1H), 7.87 (d, 1H), 8.15 (d, 1H), 8.32 (s, 1H), 8.50 (d, 1H), 8.85 (d, 1H), 9.34 (s, 1H). |
| 2-12-50 SM = 1-25-3 TBS protection group was removed during the reaction | 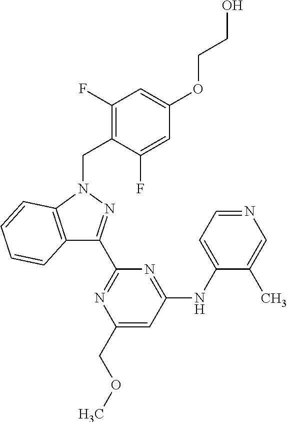 [SMILES: COCc1cc(Nc2ccncc2C)nc(-c2nn(Cc3c(F)cc(OCCO)cc3F)c3ccccc23)n1] | 2-{3,5-difluoro-4-[(3-{4- (methoxymethyl)-6-[(3- methylpyridin-4- yl)amino]pyrimidin-2- yl}-1H-indazol-1- yl)methyl]phenoxy}eth- anol | ¹H-NMR (400 MHz, DMSO-d₆): δ [ppm] = 2.29 (s, 3H), 3.46 (s, 3H), 3.67 (t, 2H), 4.00 (t, 2H), 4.53 (s, 2H), 4.90 (br. s, 1H), 5.69 (s, 2H), 6.75- 6.83 (m, 2H), 7.08 (s, 1H), 7.22 (t, 1H), 7.44-7.50 (m, 1H), 7.81 (d, 1H), 8.28-8.34 (m, 2H), 8.35 (s, 1H), 8.42 (d, 1H), 9.06 (br. s., 1H). |
| 2-12-51 SM = 1-25-3 TBS protection group was removed during the reaction | 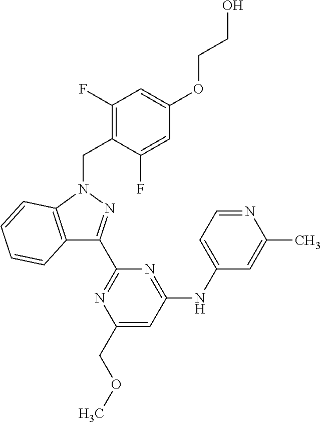 [SMILES: COCc1cc(Nc2ccnc(C)c2)nc(-c2nn(Cc3c(F)cc(OCCO)cc3F)c3ccccc23)n1] | 2-{3,5-difluoro-4-[(3-{4- (methoxymethyl)-6-[(2- methylpyridin-4- yl)amino]pyrimidin-2- yl}-1H-indazol-1- yl)methyl]phenoxy}eth- anol | ¹H-NMR (400 MHz, DMSO-d₆): δ [ppm] = 2.44 (s, 3H), 3.46 (s, 3H), 3.66 (t, 2H), 4.00 (t, 2H), 4.54 (s, 2H), 4.87 (br. s, 1H), 5.72 (s, 2H), 6.74- 6.82 (m, 2H), 6.88 (s, 1H), 7.29 (t, 1H), 7.46-7.53 (m, 1H), 7.61 (dd, 1H), 7.84 (s, 1H), 7.88 (d, 1H), 8.27 (d, 1H)), 8.52 (d, 1H), 10.00 (s, 1H). |
| 2-12-52 SM = 1-13-1 | 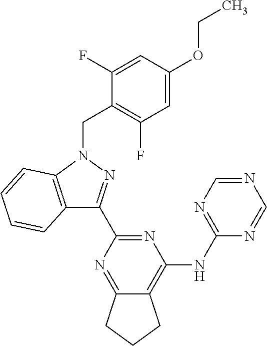 [SMILES: CCOc1cc(F)c(Cn2nc(-c3nc4c(c(Nc5ncncn5)n3)CCC4)c3ccccc32)c(F)c1] | 2-[1-(4-ethoxy-2,6- difluorobenzyl)-1H- indazol-3-yl]-N-(1,3,5- triazin-2-yl)-6,7-dihydro- 5H- cyclopenta[d]pyrimidin- 4-amine | ¹H-NMR (400 MHz, DMSO-d₆): δ [ppm] = 1.30 (t, 3 H) 2.00-2.16 (m, 2 H) 2.87 (t, 2 H) 3.06 (t, 2 H) 4.05 (q, 2 H) 5.70 (s, 2 H) 6.76 (s, 1 H) 6.79 (s, 1 H) 7.24-7.33 (m, 1 H) 7.44-7.55 (m, 1 H) 7.80 (d, 1 H) 8.78 (d, 1 H) 8.89 (s, 2 H) 10.97 (s, 1 H). |
| 2-12-53 SM = 1-13-1 | 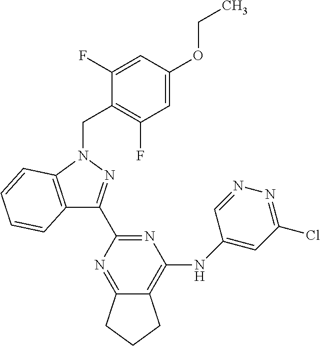 [SMILES: CCOc1cc(F)c(Cn2nc(-c3nc4c(c(Nc5cnnc(Cl)c5)n3)CCC4)c3ccccc32)c(F)c1] | N-(6-chloropyridazin-4- yl)-2-[1-(4-ethoxy-2,6- difluorobenzyl)-1H- indazol-3-yl]-6,7- dihydro-5H- cyclopenta[d]pyrimidin- 4-amine | ¹H NMR (400 MHz, DMSO-d₆) δ ppm 1.27 (t, 3 H) 2.14 (quin, 2 H) 2.93- 3.09 (m, 4 H) 4.02 (q, 2 H) 5.72 (s, 2 H) 6.73 (s, 1 H) 6.75 (s, 1 H) 7.19- 7.39 (m, 1 H) 7.40-7.64 (m, 1 H) 7.82 (d, 1 H) 8.48 (d, 1 H) 8.89 (d, 1 H) 9.46 (d, 1 H) 9.74 (br. s., 1 H) |
| 2-12-54 SM = 1-13-1 | 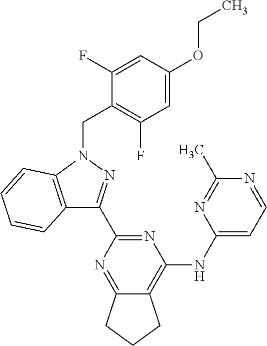 [SMILES: CCOc1cc(F)c(Cn2nc(-c3nc4c(c(Nc5ccnc(C)n5)n3)CCC4)c3ccccc32)c(F)c1] | 2-[1-(4-ethoxy-2,6- difluorobenzyl)-1H- indazol-3-yl]-N-(2- methylpyrimidin-4-yl)- 6,7-dihydro-5H- cyclopenta[d]pyrimidin- 4-amine | ¹H-NMR (400 MHz, DMSO-d₆): δ [ppm] = 1.39 (t, 3 H) 2.26 (quin, 2 H) 2.57-2.76 (m, 3 H) 2.83-3.03 (m, 2 H) 3.17 (t, 2 H) 3.98 (q, 2 H) 5.74 (s, 2 H) 6.46 (s, 1 H) 6.48 (s, 1 H) 7.28-7.30 (m, 1 H) 7.22 (br. s., 1 H) 7.45 (t, 1 H) 7.61 (d, 1 H) 8.49- 8.65 (m, 2 H) 8.72 (d, 1 H). |
| 2-12-55 SM = 1-20-7 DMF instead dioxane | 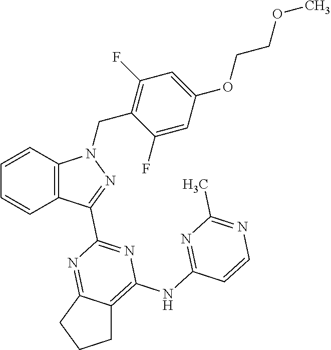 [SMILES: COCCOc1cc(F)c(Cn2nc(-c3nc4c(c(Nc5ccnc(C)n5)n3)CCC4)c3ccccc32)c(F)c1] | 2-{1-[2,6-difluoro-4-(2- methoxyethoxy)benzyl]- 1H-indazol-3-yl}-N-(2- methylpyrimidin-4-yl)- 6,7-dihydro-5H- cyclopenta[d]pyrimidin- 4-amine | ¹H-NMR (400 MHz, DMSO-d₆): δ [ppm] = 2.22-2.32 (m, 2H), 2.67 (s, 3H), 2.96 (t, 2H), 3.18 (t, 2H), 3.44 (s, 3H), 3.68- 3.76 (m, 2H), 4.04-4.10 (m, 2H), 5.75 (s, 2H), 6.47-6.57 (m, 2H), 7.17 (s, 1H), 7.25-7.32 (m, 1H), 7.45 (t, 1H), 7.61 (d, 1H), 8.55-8.62 (m, 2H), 8.71 (d, 1H). |
| 2-12-56 SM = 1-20-7 DMF instead dioxane | 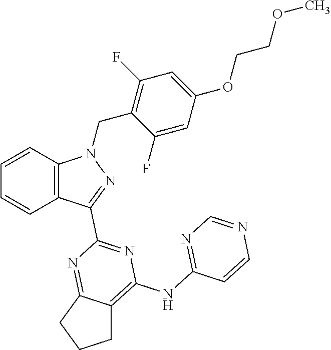 [SMILES: COCCOc1cc(F)c(Cn2nc(-c3nc4c(c(Nc5ccncn5)n3)CCC4)c3ccccc32)c(F)c1] | 2-{1-[2,6-difluoro-4-(2- methoxyethoxy)benzyl]- 1H-indazol-3-yl}-N- (pyrimidin-4-yl)-6,7- dihydro-5H- cyclopenta[d]pyrimidin- 4-amine | ¹H-NMR (400 MHz, DMSO-d₆): δ [ppm] = 2.03-2.15 (m, 2H), 2.97- 3.05 (m, 4H), 3.27 (s, 3H), 3.59-3.65 (m, 2H), 4.09-4.15 (m, 2H), 5.72 (s, 2H), 6.82-6.92 (m, 2H), 7.26-7.33 (m, 1H), 7.47-7.54 (m, 1H), 7.87 (d, 1H), 8.53 (d, 1H), 8.57 (d, 1H), 8.66 (dd, 1H), 8.86 (d, 1H), 10.08 (s, 1H). |
| 2-12-57 SM = 1-20-7 DMF instead dioxane | 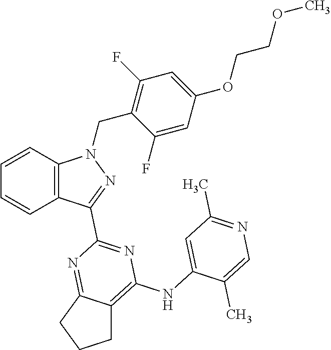 [SMILES: COCCOc1cc(F)c(Cn2nc(-c3nc4c(c(Nc5cc(C)ncc5C)n3)CCC4)c3ccccc32)c(F)c1] | 2-{1-[2,6-difluoro-4-(2- methoxyethoxy)benzyl]- 1H-indazol-3-yl}-N-(2,5- dimethylpyridin-4-yl)- 6,7-dihydro-5H- cyclopenta[d]pyrimidin- 4-amine | ¹H-NMR (400 MHz, DMSO-d₆): δ [ppm] = 2.22-2.30 (m, 2H), 2.31 (s, 3H), 2.59 (s, 3H), 2.90 (t, 2H), 3.19 (t, 2H), 3.44 (s, 3H), 3.70-3.75 (m, 2H), 4.05-4.09 (m, 2H), 5.78 (s, 2H), 6.29 (s, 1H), 6.47- 6.56 (m, 2H), 7.22- 7.27 (m, 1H), 7.38-7.45 (m, 1H), 7.54 (d, 1H), 8.30 (s, 1H), 8.45 (s, 1H), 8.62 (d, 1H). |
| 2-12-58 SM = 1-20-7 DMF instead dioxane | 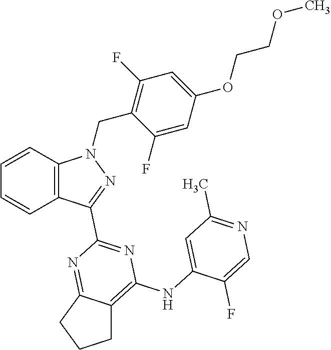 [SMILES: COCCOc1cc(F)c(Cn2nc(-c3nc4c(c(Nc5cc(C)ncc5F)n3)CCC4)c3ccccc32)c(F)c1] | 2-{1-[2,6-difluoro-4-(2- methoxyethoxy)benzyl]- 1H-indazol-3-yl}-N-(5- fluoro-2-methylpyridin- 4-yl)-6,7-dihydro-5H- cyclopenta[d]pyrimidin- 4-amine | ¹H-NMR (400 MHz, DMSO-d₆): δ [ppm] = 2.11 (quin, 2H), 2.46 (s, 3H), 2.97 (q, 4H), 3.27 (s, 3H), 3.58-3.66 (m, 2H), 4.08-4.14 (m, 2H), 5.70 (s, 2H), 6.75-6.85 (m, 2H), 7.18 (t, 1H), 7.47 (t, 1H), 7.80 (d, 1H), 8.14 (d, 1H), 8.33 (d, 1H), 8.38- 8.42 (m, 1H), 8.85 (s, 1H). |
| 2-12-59 SM = 1-20-8 DMF instead dioxane | 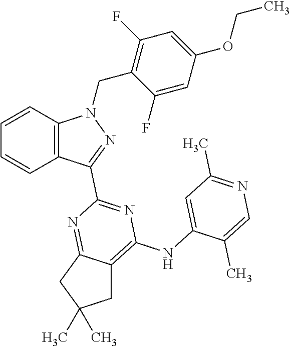 [SMILES: CCOc1cc(F)c(Cn2nc(-c3nc4c(c(Nc5cc(C)ncc5C)n3)CC(C)(C)C4)c3ccccc32)c(F)c1] | N-(2,5-dimethylpyridin- 4-yl)-2-[1-(4-ethoxy-2,6- difluorobenzyl)-1H- indazol-3-yl]-6,6- dimethyl-6,7-dihydro- 5H- cyclopenta[d]pyrimidin- 4-amine | ¹H-NMR (400 MHz, DMSO-d₆): δ [ppm] = 1.22 (s, 6H), 1.30 (t, 3H), 2.22 (s, 3H), 2.44 (s, 3H), 2.73 (s, 2H), 2.80 (s, 2H), 4.05 (q, 2H), 5.67 (s, 2H), 6.71-6.82 (m, 2H), 7.11 (t, 1H), 7.41-7.48 (m, 1H), 7.73 (s, 1H), 7.78 (d, 1H), 8.21-8.26 (m, 2H), 8.28 (s, 1H). |
| 2-12-60 SM = 1-20-8 DMF instead dioxane | 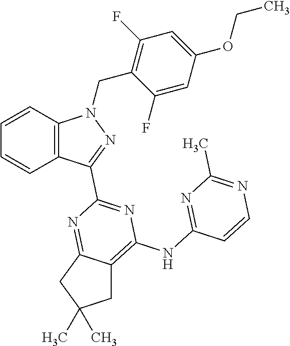 [SMILES: CCOc1cc(F)c(Cn2nc(-c3nc4c(c(Nc5ccnc(C)n5)n3)CC(C)(C)C4)c3ccccc32)c(F)c1] | 2-[1-(4-ethoxy-2,6- difluorobenzyl)-1H- indazol-3-yl]-6,6- dimethyl-N-(2- methylpyrimidin-4-yl)- 6,7-dihydro-5H- cyclopenta[d]pyrimidin- 4-amine | ¹H-NMR (400 MHz, DMSO-d₆): δ [ppm] = 1.18 (s, 6H), 1.29 (t, 3H), 2.55 (s, 3H), 2.81 (s, 2H), 2.84 (s, 2H), 4.04 (q, 2H), 5.71 (s, 2H), 6.76-6.87 (m, 2H), 7.28 (t, 1H), 7.47- 7.54 (m, 1H), 7.86 (d, 1H), 8.37 (d, 1H), 8.45 (d, 1H), 8.51 (d, 1H), 9.96 (s, 1H). |
| 2-12-61 SM = 1-20-9 DMF instead dioxane | 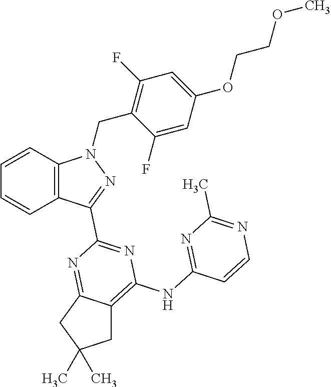 [SMILES: COCCOc1cc(F)c(Cn2nc(-c3nc4c(c(Nc5ccnc(C)n5)n3)CC(C)(C)C4)c3ccccc32)c(F)c1] | 2-{1-[2,6-difluoro-4-(2- methoxyethoxy)benzyl]- 1H-indazol-3-yl}-6,6- dimethyl-N-(2- methylpyrimidin-4-yl)- 6,7-dihydro-5H- cyclopenta[d]pyrimidin- 4-amine | ¹H-NMR (400 MHz, DMSO-d₆): δ [ppm] = 1.18 (s, 6H), 2.55 (s, 3H), 2.81 (s, 2H), 2.84 (s, 2H), 3.27 (s, 3H), 3.58-3.66 (m, 2H), 4.09- 4.16 (m, 2H), 5.71 (s, 2H), 6.80-6.89 (m, 2H), 7.28 (t, 1H), 7.47-7.54 (m, 1H), 7.86 (d, 1H), 8.37 (d, 1H), 8.45 (d, 1H), 8.51 (d, 1H), 9.96 (s, 1H). |
| 2-12-62 SM = 1-35-1 | 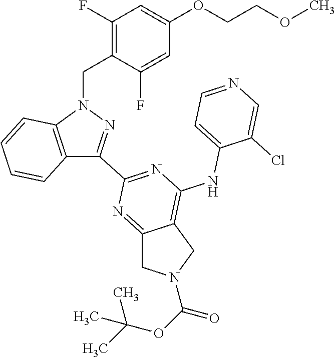 [SMILES: COCCOc1cc(F)c(Cn2nc(-c3nc4c(c(Nc5ccncc5Cl)n3)CN(C(=O)OC(C)(C)C)C4)c3ccccc32)c(F)c1] | tert-butyl 4-[(3- chloropyridin-4- yl)amino]-2-{1-[2,6- difluoro-4-(2- methoxyethoxy)benzyl]- 1H-indazol-3-yl}-5,7- dihydro-6H-pyrrolo[3,4- d]pyrimidine-6- carboxylate | ¹H-NMR (400 MHz, DMSO-d₆): δ [ppm] = 1.48/1.50 (s, 9H), 3.27 (s, 3H), 3.62 (m, 2H), 4.12 (m, 2H), 4.60/4.64 (br. s., 2H), 4.64/4.69 (br. s., 2H), 5.68 (s, 2H), 6.81 (m, 2H), 7.09/7.13 (dd, 1H), 7.44/7.46 (dd, 1H), 7.79/7.81 (d, 1H), 8.00/8.14 (d, 1H), 8.05/8.15 (d, 1H), 8.49/8.49 (d, 1 H), 8.69/8.70 (s, 1H), 9.17/9.29 (br. s., 1H). |
| 2-12-63 SM = 1-35-1 | 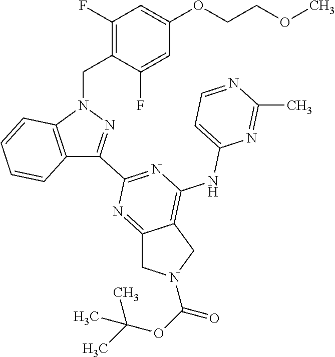 [SMILES: COCCOc1cc(F)c(Cn2nc(-c3nc4c(c(Nc5ccnc(C)n5)n3)CN(C(=O)OC(C)(C)C)C4)c3ccccc32)c(F)c1] | tert-butyl 2-{1-[2,6- difluoro-4-(2- methoxyethoxy)benzyl]- 1H-indazol-3-yl}-4-[(2- methylpyrimidin-4- yl)amino]-5,7-dihydro- 6H-pyrrolo[3,4- d]pyrimidine-6- carboxylate | ¹H-NMR (400 MHz, DMSO-d₆): δ [ppm] = 1.48/1.49 (s, 9H), 2.56 (s, 3H), 3.26 (s, 3H), 3.61 (m, 2H), 4.11 (m, 2H), 4.65/4.61 (br. s., 2H), 4.73/4.69 (br. s., 2H), 5.72 (s, 2H), 6.85 (m, 2H), 7.30/7.30 (dd, 1H), 7.51 (dd, 1H), 7.88 (d, 1H), 8.44 (d, 1H), 8.50 (d, 1H), 8.50 (d, 1H), 10.45/10.50 (br. s., 1H). |
| 2-12-64 SM = 1-35-1 | 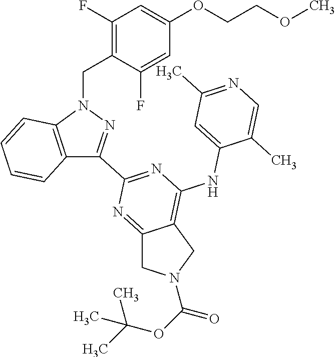 [SMILES: COCCOc1cc(F)c(Cn2nc(-c3nc4c(c(Nc5cc(C)ncc5C)n3)CN(C(=O)OC(C)(C)C)C4)c3ccccc32)c(F)c1] | tert-butyl 2-{1-[2,6- difluoro-4-(2- methoxyethoxy)benzyl]- 1H-indazol-3-yl}-4-[(2,5- dimethylpyridin-4- yl)amino]-5,7-dihydro- 6H-pyrrolo[3,4- d]pyrimidine-6- carboxylate | ¹H-NMR (400 MHz, DMSO-d₆): δ [ppm] = 1.48/1.49 (s, 9H), 2.19/2.20 (s, 3H), 2.45 (s, 3H), 3.27 (s, 3H), 3.62 (m, 2H), 4.11 (m, 2H), 4.57/4.59 (br. s., 2H), 4.59/4.63 (br. s., 2H), 5.67 (s, 2H), 6.80 (m, 2H), 7.06/7.10 (dd, 1H), 7.43/7.44 (dd, 1H), 7.48/7.56 (s, 1H), 7.77/7.78 (d, 1H), 8.07/8.16 (d, 1H), 8.33/8.34 (s, 1H), 8.86/8.96 (br. s., 1 H). |
| 2-12-65 SM = 1-20-8 DMF instead dioxane | 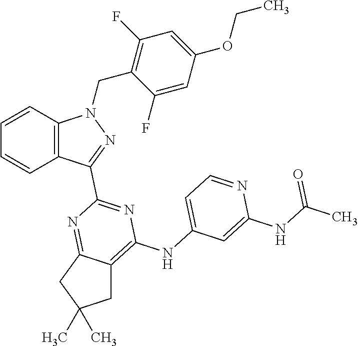 [SMILES: CCOc1cc(F)c(Cn2nc(-c3nc4c(c(Nc5ccnc(NC(C)=O)c5)n3)CC(C)(C)C4)c3ccccc32)c(F)c1] | N-[4-({2-[1-(4-ethoxy- 2,6-difluorobenzyl)-1H- indazol-3-yl]-6,6- dimethyl-6,7-dihydro- 5H- cyclopenta[d]pyrimidin- 4-yl}amino)pyridin-2- yl]acetamide | ¹H-NMR (400 MHz, DMSO-d₆): δ [ppm] = 1.21 (s, 6H), 1.29 (t, 3H), 2.09 (s, 3H), 2.79 (s, 2H), 2.81 (s, 2H), 4.03 (q, 2H), 5.70 (s, 2H), 6.79 (d, 2H), 7.24 (t, 1H), 7.48 (t, 1H), 7.83 (d, 1H), 8.13 (d, 1H), 8.19 (s, 1H), 8.34 (d, 1H), 8.48 (d, 1H), 9.37 (s, 1H), 10.34 (s, 1H). |
| 2-12-66 SM = 1-20-10 DMF instead dioxane | 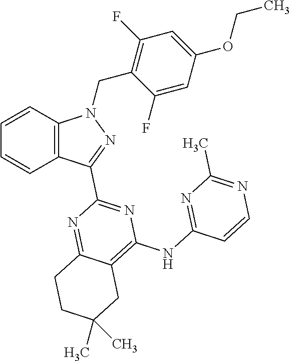 [SMILES: CCOc1cc(F)c(Cn2nc(-c3nc4c(c(Nc5ccnc(C)n5)n3)CC(C)(C)CC4)c3ccccc32)c(F)c1] | 2-[1-(4-ethoxy-2,6- difluorobenzyl)-1H- indazol-3-yl]-6,6- dimethyl-N-(2- methylpyrimidin-4-yl)- 5,6,7,8- tetrahydroquinazolin-4- amine | ¹H-NMR (400 MHz, DMSO-d₆): δ [ppm] = 1.03 (s, 6H), 1.29 (t, 3H), 1.65 (t, 2H), 2.54-2.61 (m, 5H), 2.90 (t, 2H), 4.04 (q, 2H), 5.71 (s, 2H), 6.76- 6.86 (m, 2H), 7.23-7.32 (m, 1H), 7.46-7.55 (m, 1H), 7.85 (d, 1H), 8.40 (d, 1H), 8.45 (d, 1H), 8.51 (d, 1H), 9.16 (s, 1H). |
| 2-12-67 SM = 1-20-10 DMF instead dioxane | 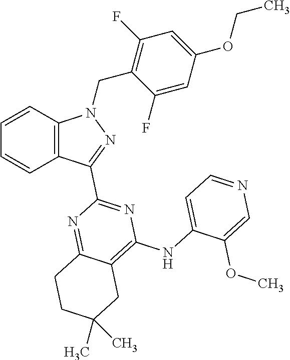 [SMILES: CCOc1cc(F)c(Cn2nc(-c3nc4c(c(Nc5ccncc5OC)n3)CC(C)(C)CC4)c3ccccc32)c(F)c1] | 2-[1-(4-ethoxy-2,6- difluorobenzyl)-1H- indazol-3-yl]-N-(3- methoxypyridin-4-yl)- 6,6-dimethyl-5,6,7,8- tetrahydroquinazolin-4- amine | ¹H-NMR (400 MHz, DMSO-d₆): δ [ppm] = 1.07 (s, 6H), 1.29 (t, 3H), 1.66 (t, 2H), 2.45 (s, 2H), 2.88 (t, 2H), 4.01 (s, 3H), 4.04 (q, 2H), 5.70 (s, 2H), 6.76- 6.85 (m, 2H), 7.25 (t, 1H), 7.46-7.52 (m, 1H), 7.73 (s, 1H), 7.86 (d, 1H), 8.18 (d, 1H), 8.36 (s, 1H), 8.43 (d, 1H), 8.75 (d, 1H). |
| 2-12-68 SM = 1-20-10 DMF instead dioxane | 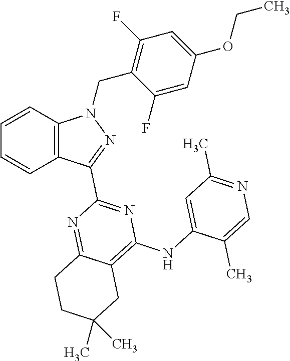 [SMILES: CCOc1cc(F)c(Cn2nc(-c3nc4c(c(Nc5cc(C)ncc5C)n3)CC(C)(C)CC4)c3ccccc32)c(F)c1] | N-(2,5-dimethylpyridin- 4-yl)-2-[1-(4-ethoxy-2,6- difluorobenzyl)-1H- indazol-3-yl]-6,6- dimethyl-5,6,7,8- tetrahydroquinazolin-4- amine | ¹H-NMR (400 MHz, DMSO-d₆): δ [ppm] = 1.07 (s, 6H), 1.29 (t, 3H), 1.66 (t, 2H), 2.18 (s, 3H), 2.44 (s, 3H), 2.84 (t, 2H), 4.04 (q, 2H), 5.67 (s, 2H), 6.71- 6.80 (m, 2H), 7.04- 7.11 (m, 1H), 7.39-7.46 (m, 1H), 7.72-7.78 (m, 2H), 7.96 (s, 1H), 8.13 (d, 1H), 8.29 (s, 1H). - one CH₂ group not detectable |
| 2-12-69 SM = 1-20-10 DMF instead dioxane | 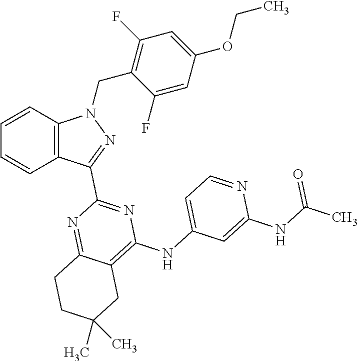 [SMILES: CCOc1cc(F)c(Cn2nc(-c3nc4c(c(Nc5ccnc(NC(C)=O)c5)n3)CC(C)(C)CC4)c3ccccc32)c(F)c1] | N-[4-({2-[1-(4-ethoxy- 2,6-difluorobenzyl)-1H- indazol-3-yl]-6,6- dimethyl-5,6,7,8- tetrahydroquinazolin-4- yl}amino)pyridin-2- yl]acetamide | ¹H-NMR (400 MHz, DMSO-d₆): δ [ppm] = 1.06 (s, 6H), 1.29 (t, 3H), 1.65 (t, 2H), 2.09 (s, 3H), 2.86 (t, 2H), 4.04 (q, 2H), 5.70 (s, 2H), 6.74-6.83 (m, 2H), 7.17-7.24 (m, 1H), 7.44-7.50 (m, 1H), 7.81 (d, 1H), 8.12 (d, 1H), 8.17-8.22 (m, 1H), 8.27 (br. s, 1H), 8.46 (d, 1H), 8.92 (s, 1H), 10.34 (s, 1H). 2H not detectable. |
| 2-12-70 SM = 1-20-11 DMF instead dioxane | 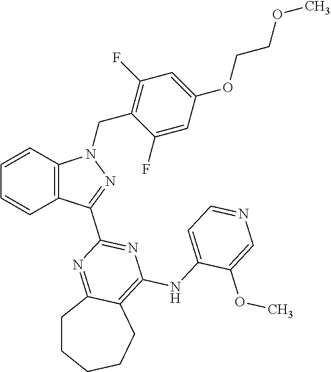 [SMILES: COCCOc1cc(F)c(Cn2nc(-c3nc4c(c(Nc5ccncc5OC)n3)CCCCC4)c3ccccc32)c(F)c1] | 2-{1-[2,6-difluoro-4-(2- methoxyethoxy)benzyl]- 1H-indazol-3-yl}-N-(3- methoxypyridin-4-yl)- 6,7,8,9-tetrahydro-5H- cyclohepta[d]pyrimidin- 4-amine | ¹H-NMR (400 MHz, DMSO-d₆): δ [ppm] = 1.57-1.75 (m, 4H), 1.83- 1.94 (m, 2H), 2.78-2.86 (m, 2H), 3.00-3.07 (m, 2H), 3.26 (s, 3H), 3.59- 3.66 (m, 2H), 3.97 (s, 3H), 4.09-4.16 (m, 2H), 5.70 (s, 2H), 6.78-6.89 (m, 2H), 7.21 (t, 1H), 7.44-7.52 (m, 1H), 7.83 (d, 1H), 8.12 (s, 1H), 8.17 (d, 1H), 8.34 (s, 1H), 8.40 (d, 1H), 8.54 (d, 1H). |
| 2-12-71 SM = 1-20-12 DMF instead dioxane | 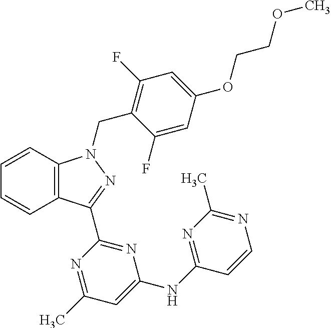 [SMILES: COCCOc1cc(F)c(Cn2nc(-c3nc(C)cc(Nc4ccnc(C)n4)n3)c3ccccc32)c(F)c1] | 2-{1-[2,6-difluoro-4-(2- methoxyethoxy)benzyl]- 1H-indazol-3-yl}-6- methyl-N-(2- methylpyrimidin-4- yl)pyrimidin-4-amine | ¹H-NMR (400 MHz, DMSO-d₆): δ [ppm] = 2.52 (s, 3H), 2.56 (s, 3H), 3.27 (s, 3H), 3.57-3.65 (m, 2H), 4.08-4.17 (m, 2H), 5.73 (s, 2H), 6.80-6.90 (m, 2H), 7.28-7.35 (m, 1H), 7.48-7.56 (m, 2H), 7.85 (d, 1H), 7.96 (d, 1H), 8.44 (d, 1H), 8.62 (d, 1H), 10.61 (s, 1H). |
| 2-12-72 SM = 1-20-13 DMF instead dioxane | 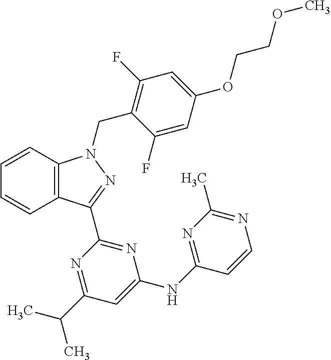 [SMILES: COCCOc1cc(F)c(Cn2nc(-c3nc(Nc4ccnc(C)n4)cc(C(C)C)n3)c3ccccc32)c(F)c1] | 2-{1-[2,6-difluoro-4-(2- methoxyethoxy)benzyl]- 1H-indazol-3-yl}-6- isopropyl-N-(2- methylpyrimidin-4- yl)pyrimidin-4-amine | ¹H-NMR (400 MHz, DMSO-d₆): δ [ppm] = 1.35 (d, 6H), 2.56 (s, 3H), 2.96-3.08 (m, 1H), 3.27 (s, 3H), 3.58-3.64 (m, 2H), 4.05-4.15 (m, 2H), 5.73 (s, 2H), 6.81-6.89 (m, 2H), 7.30-7.36 (m, 1H), 7.47- 7.55 (m, 1H), 7.61 (br. s, 1H), 7.85 (d, 1H), 7.98 (br. d, 1H), 8.44 (d, 1H), 8.61 (d, 1H), 10.64 (s, 1H). |
| 2-12-73 SM = 1-20-13 DMF instead dioxane | 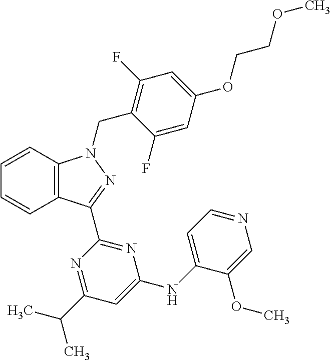 [SMILES: COCCOc1cc(F)c(Cn2nc(-c3nc(Nc4ccncc4OC)cc(C(C)C)n3)c3ccccc32)c(F)c1] | 2-{1-[2,6-difluoro-4-(2- methoxyethoxy)benzyl]- 1H-indazol-3-yl}-6- isopropyl-N-(3- methoxypyridin-4- yl)pyrimidin-4-amine | ¹H-NMR (400 MHz, DMSO-d₆): δ [ppm] = 1.33 (d, 6H), 2.90-3.02 (m, 1H), 3.26 (s, 3H), 3.58-3.65 (m, 2H), 4.00 (s, 3H), 4.08-4.15 (m, 2H), 5.73 (s, 2H), 6.79- 6.91 (m, 2H), 7.15 (s, 1H), 7.27-7.34 (m, 1H), 7.48- 7.55 (m, 1H), 7.88 (d, 1H), 8.14 (d, 1H), 8.30 (s, 1H), 8.57 (d, 1H), 8.96 (d, 1H), 9.16 (s, 1H). |
| 2-12-74 SM = 1-20-13 DMF instead dioxane | 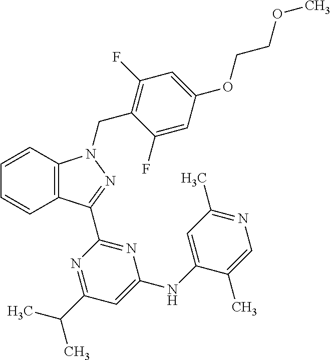 [SMILES: COCCOc1cc(F)c(Cn2nc(-c3nc(Nc4cc(C)ncc4C)cc(C(C)C)n3)c3ccccc32)c(F)c1] | 2-{1-[2,6-difluoro-4-(2- methoxyethoxy)benzyl]- 1H-indazol-3-yl}-N- (2,5-dimethylpyridin-4- yl)-6- isopropylpyrimidin-4- amine | ¹H-NMR (400 MHz, DMSO-d₆): δ [ppm] = 1.33 (d, 6H), 2.26 (s, 3H), 2.42 (s, 3H), 2.91-3.05 (m, 1H), 3.27 (s, 3H), 3.58- 3.65 (m, 2H), 4.06-4.16 (m, 2H), 5.73 (s, 2H), 6.76-6.85 (m, 2H), 6.95 (s, 1H), 7.23-7.32 (m, 1H), 7.44-7.53 (m, 1H), 7.83 (d, 1H), 8.20 (s, 1H), 8.30 (s, 1H), 8.54 (d, 1H), 8.84 (s, 1H). |
| 2-12-75 SM = 1-20-14 DMF instead dioxane | 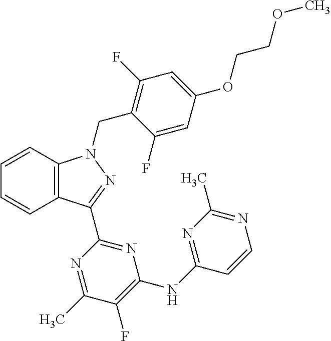 [SMILES: COCCOc1cc(F)c(Cn2nc(-c3nc(C)c(F)c(Nc4ccnc(C)n4)n3)c3ccccc32)c(F)c1] | 2-{1-[2,6-difluoro-4-(2- methoxyethoxy)benzyl]- 1H-indazol-3-yl}-5- fluoro-6-methyl-N-(2- methylpyrimidin-4- yl)pyrimidin-4-amine | ¹H-NMR (400 MHz, DMSO-d₆): δ [ppm] = 2.52-2.55 (m, 3H), 2.57 (s, 3H), 3.27 (s, 3H), 3.58- 3.64 (m, 2H), 4.09-4.16 (m, 2H), 5.72 (s, 2H), 6.81-6.90 (m, 2H), 7.27- 7.34 (m, 1H), 7.47-7.54 (m, 1H), 7.87 (d, 1H), 8.38 (d, 1H), 8.49-8.55 (m, 2H), 10.47 (s, 1H). |
| 2-12-76 SM = 1-20-15 DMF instead dioxane | 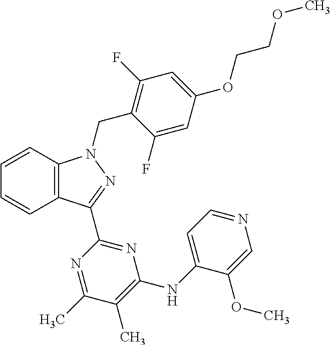 [SMILES: COCCOc1cc(F)c(Cn2nc(-c3nc(C)c(C)c(Nc4ccncc4OC)n3)c3ccccc32)c(F)c1] | 2-{1-[2,6-difluoro-4-(2- methoxyethoxy)benzyl]- 1H-indazol-3-yl}-N-(3- methoxypyridin-4-yl)- 5,6-dimethylpyrimidin- 4-amine | ¹H-NMR (400 MHz, DMSO-d₆): δ [ppm] = 2.24 (s, 3H), 2.50 (br. s., 3H), 3.26 (s, 3H), 3.58- 3.65 (m, 2H), 3.99 (s, 3H), 4.08-4.15 (m, 2H), 5.71 (s, 2H), 6.80-6.89 (m, 2H), 7.21-7.29 (m, 1H), 7.45-7.53 (m, 1H), 7.79- 7.89 (m, 2H), 8.18 (d, 1H), 8.34 (s, 1H), 8.46 (d, 1H), 8.75 (d, 1H). |
| 2-12-77 SM = 1-20-12 DMF instead dioxane | 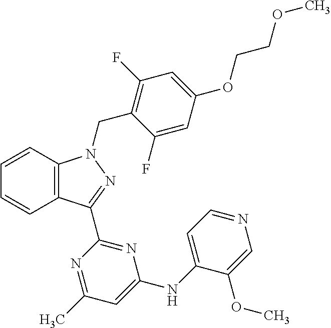 [SMILES: COCCOc1cc(F)c(Cn2nc(-c3nc(C)cc(Nc4ccncc4OC)n3)c3ccccc32)c(F)c1] | 2-{1-[2,6-difluoro-4-(2- methoxyethoxy)benzyl]- 1H-indazol-3-yl}-N-(3- methoxypyridin-4-yl)-6- methylpyrimidin-4- amine | ¹H-NMR (400 MHz, DMSO-d₆): δ [ppm] = 2.46 (s, 3H), 3.26 (s, 3H), 3.58-3.67 (m, 2H), 3.99 (s, 3H), 4.08-4.17 (m, 2H), 5.72 (s, 2H), 6.77-6.91 (m, 2H), 7.07 (s, 1H), 7.29 (t, 1H), 7.46-7.55 (m, 1H), 7.87 (d, 1H), 8.14 (d, 1H), 8.31 (s, 1H), 8.54 (d, 1H), 8.83 (d, 1H), 9.15 (s, 1H). |
| 2-12-78 SM = 1-20-12 N,N- Diisopro- pylethyl- amin as base | 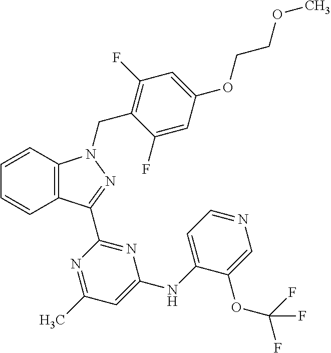 [SMILES: COCCOc1cc(F)c(Cn2nc(-c3nc(C)cc(Nc4ccncc4OC(F)(F)F)n3)c3ccccc32)c(F)c1] | 2-{1-[2,6-difluoro-4-(2- methoxyethoxy)benzyl]- 1H-indazol-3-yl}-6- methyl-N-[3- (trifluoromethoxy)pyr- idin-4-yl]pyrimidin-4- amine | ¹H-NMR (400 MHz, DMSO-d₆): δ [ppm] = 2.51 (s, 3H), 3.25 (s, 3H), 3.61 (m, 2H), 4.11 (m, 2H), 5.72 (s, 2H), 6.84 (m, 2H), 7.11 (s, 1H), 7.29 (dd, 1H), 7.50 (dd, 1H), 7.86 (d, 1H), 8.43 (d, 1H), 8.51 (d, 1H), 8.57 (br. s., 1H), 9.04 (d, 1H), 9.73 (s, 1H). |
| 2-12-79 SM = 1-13-2 N,N- Diisopro- pylethyl- amin as base | 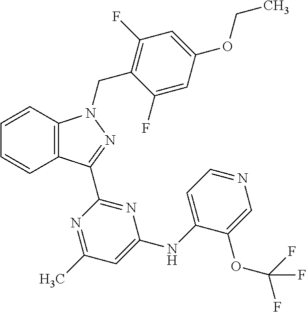 [SMILES: CCOc1cc(F)c(Cn2nc(-c3nc(C)cc(Nc4ccncc4OC(F)(F)F)n3)c3ccccc32)c(F)c1] | 2-[1-(4-ethoxy-2,6- difluorobenzyl)-1H- indazol-3-yl]-6-methyl- N-[3- (trifluoromethoxy)pyr- idin-4-yl]pyrimidin-4- amine | ¹H-NMR (400 MHz, DMSO-d₆): δ [ppm] = 1.29 (t, 3H), 2.50 (s, 3H), 4.04 (q, 2H), 5.72 (s, 2H), 6.81 (m, 2H), 7.11 (s, 1H), 7.29 (dd, 1H), 7.51 (dd, 1H), 7.87 (d, 1H), 8.42 (d, 1H), 8.51 (d, 1H), 8.57 (br. s., 1H), 9.04 (d, 1H), 9.74 (s, 1H). |
| 2-12-80 SM = 1-13-2 N,N- Diisopro- pylethyl- amin as base | 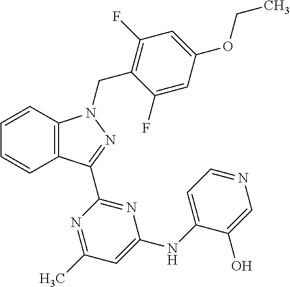 [SMILES: CCOc1cc(F)c(Cn2nc(-c3nc(C)cc(Nc4ccncc4O)n3)c3ccccc32)c(F)c1] | 4-({2-[1-(4-ethoxy-2,6- difluorobenzyl)-1H- indazol-3-yl]-6- methylpyrimidin-4- yl}amino)pyridin-3-ol | ¹H-NMR (400 MHz, DMSO-d₆): δ [ppm] = 1.26 (t, 3H), 2.42 (s, 3H), 4.02 (q, 2H), 5.68 (s, 2H), 6.77 (m, 2H), 7.00 (s, 1H), 7.25 (dd, 1H), 7.47 (dd, 1H), 7.83 (d, 1H), 7.94 (d, 1H), 8.09 (br. s., 1H), 8.51 (d, 1H), 8.57 (d, 1H), 9.12 (s, 1H), 10.42 (br. s., 1H). |
| 2-12-81 SM = 1-20-16 | 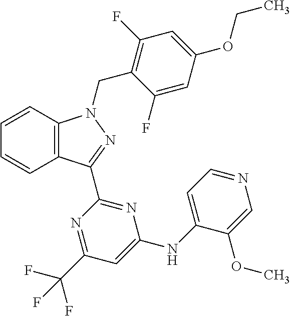 [SMILES: CCOc1cc(F)c(Cn2nc(-c3nc(Nc4ccncc4OC)cc(C(F)(F)F)n3)c3ccccc32)c(F)c1] | 2-[1-(4-ethoxy-2,6- difluorobenzyl)-1H- indazol-3-yl]-N-(3- methoxypyridin-4-yl)-6- (trifluoromethyl)pyr- imidin-4-amine | ¹H-NMR (400 MHz, DMSO-d₆): δ [ppm] = 1.29 (t, 3H), 3.98-4.08 (m, 5H), 5.75 (s, 2H), 6.77- 6.86 (m, 2H), 7.30-7.37 (m, 1H), 7.51-7.58 (m, 1H), 7.69 (s, 1H), 7.91 (d, 1H), 8.20 (d, 1H), 8.39 (s, 1H), 8.43 (d, 1H), 8.85 (d, 1H), 9.84 (br. s, 1H). |
| 2-12-82 SM = 1-20-16 | 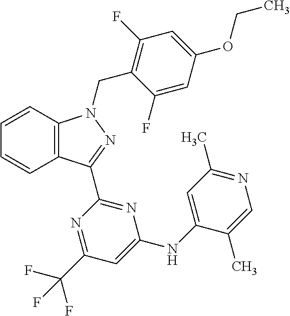 [SMILES: CCOc1cc(F)c(Cn2nc(-c3nc(Nc4cc(C)ncc4C)cc(C(F)(F)F)n3)c3ccccc32)c(F)c1] | N-(2,5-dimethylpyridin- 4-yl)-2-[1-(4-ethoxy-2,6- difluorobenzyl)-1H- indazol-3-yl]-6- (trifluoromethyl)pyr- imidin-4-amine | ¹H-NMR (400 MHz, DMSO-d₆): δ [ppm] = 1.29 (t, 3H), 2.25 (s, 3H), 2.44 (s, 3H), 4.04 (q, 2H), 5.72-5.77 (m, 2H), 6.72- 6.82 (m, 2H), 7.21-7.29 (m, 1H), 7.28-7.35 (m, 1H), 7.46-7.53 (m, 1H), 7.83 (d, 1H), 8.07 (br. s, 1H), 8.26 (s, 1H), 8.38 (d, 1H), 9.53 (br. s, 1H). |
| 2-12-83 SM = 1-20-16 | 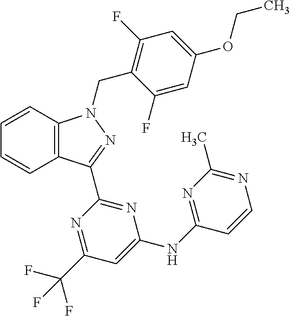 [SMILES: CCOc1cc(F)c(Cn2nc(-c3nc(Nc4ccnc(C)n4)cc(C(F)(F)F)n3)c3ccccc32)c(F)c1] | 2-[1-(4-ethoxy-2,6- difluorobenzyl)-1H- indazol-3-yl]-N-(2- methylpyrimidin-4-yl)- 6-(trifluoromethyl)pyr- imidin-4-amine | ¹H-NMR (400 MHz, DMSO-d₆): δ [ppm] = 1.29 (t, 3H), 2.59 (s, 3H), 4.04 (q, 2H), 5.77 (s, 2H), 6.77-6.86 (m, 2H), 7.35- 7.41 (m, 1H), 7.51-7.58 (m, 1H), 7.90 (d, 1H), 7.97 (br. s, 1H), 8.10 (br. s, 1H), 8.49-8.57 (m, 2H), 11.22 (s, 1H). |
| 2-12-84 SM = 1-35-2 N,N- Diisopro- pylethyl- amine as base | 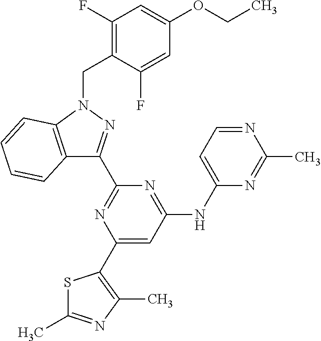 [SMILES: CCOc1cc(F)c(Cn2nc(-c3nc(Nc4ccnc(C)n4)cc(-c4sc(C)nc4C)n3)c3ccccc32)c(F)c1] | 6-(2,4-dimethyl-1,3- thiazol-5-yl)-2-[1-(4- ethoxy-2,6- difluorobenzyl)-1H- indazol-3-yl]-N-(2- methylpyrimidin-4- yl)pyrimidin-4-amine | ¹H-NMR (400 MHz, DMSO-d₆): δ [ppm] = 1.29 (t, 3H), 2.57 (s, 3H), 2.70 (s, 3H), 2.75 (s, 3H), 4.04 (q, 2H), 5.73 (s, 2H), 6.81 (m, 2H), 7.37 (dd, 1H), 7.53 (dd, 1H), 7.77 (br. s., 1H), 7.86 (d, 1H), 8.31 (m, 1H), 8.45 (d, 1H), 8.62 (d, 1H), 10.84 (br. s., 1H). |
| 2-12-85 SM = 1-35-2 N,N- Diisopro- pylethyl- amine as base | 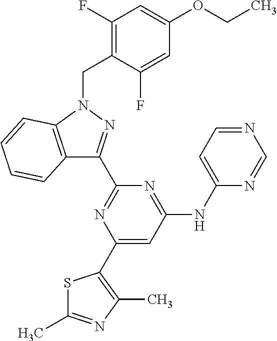 [SMILES: CCOc1cc(F)c(Cn2nc(-c3nc(Nc4ccncn4)cc(-c4sc(C)nc4C)n3)c3ccccc32)c(F)c1] | 6-(2,4-dimethyl-1,3- thiazol-5-yl)-2-[1-(4- ethoxy-2,6- difluorobenzyl)-1H- indazol-3-yl]-N- (pyrimidin-4- yl)pyrimidin-4-amine | ¹H-NMR (400 MHz, DMSO-d₆): δ [ppm] = 1.29 (t, 3H), 2.70 (s, 3H), 2.72 (s, 3H), 4.04 (q, 2H), 5.74 (s, 2H), 6.82 (m, 2H), 7.37 (dd, 1H), 7.53 (dd, 1H), 7.88 (d, 1H), 8.06 (br. s., 1H), 8.19 (m, 1H), 8.57 (d, 1H), 8.60 (d, 1H), 8.87 (s, 1H), 10.89 (br. s., 1H). |
| 2-12-86 SM = 1-35-3 N,N- Diisopro- pylethyl- amine as base | 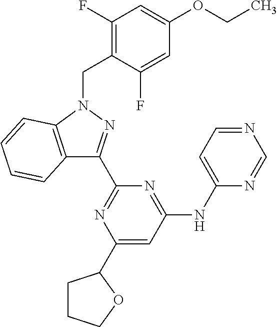 [SMILES: CCOc1cc(F)c(Cn2nc(-c3nc(Nc4ccncn4)cc(C4CCCO4)n3)c3ccccc32)c(F)c1] | 2-[1-(4-ethoxy-2,6- difluorobenzyl)-1H- indazol-3-yl]-N- (pyrimidin-4-yl)-6- [(2rac)-tetrahydrofuran- 2-yl]pyrimidin-4-amine | ¹H-NMR (400 MHz, DMSO-d₆): δ [ppm] = 1.29 (t, 3H), 1.99 (m, 3H), 2.46 (m, 1H), 3.93 (m, 1H), 4.03 (q, 2H), 4.03 (m, 1H), 4.96 (m, 1H), 5.73 (s, 2H), 6.81 (m, 2H), 7.32 (dd, 1H), 7.51 (dd, 1H), 7.72 (s, 1H), 7.85 (d, 1H), 8.16 (d, 1H), 8.54 (d, 1H), 8.55 (d, 1H), 8.85 (s, 1H), 10.74 (br. s., 1H). |
| 2-12-87 SM = 1-35-3 N,N- Diisopro- pylethyl- amine as base | 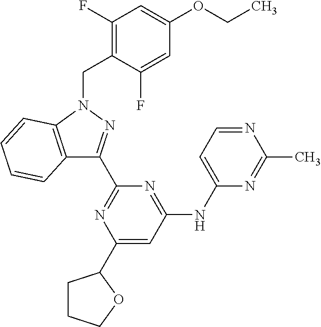 [SMILES: CCOc1cc(F)c(Cn2nc(-c3nc(Nc4ccnc(C)n4)cc(C4CCCO4)n3)c3ccccc32)c(F)c1] | 2-[1-(4-ethoxy-2,6- difluorobenzyl)-1H- indazol-3-yl]-N-(2- methylpyrimidin-4-yl)-6- [(2rac)-tetrahydrofuran- 2-yl]pyrimidin-4-amine | ¹H-NMR (400 MHz, DMSO-d₆): δ [ppm] = 1.29 (t, 3H), 1.98 (m, 3H), 2.45 (m, 1H), 2.55 (s, 3H), 3.95 (m, 1H), 4.03 (q, 2H), 4.03 (m, 1H), 4.96 (m, 1H), 5.72 (s, 2H), 6.80 (m, 2H), 7.31 (dd, 1H), 7.51 (dd, 1H), 7.73 (s, 1H), 7.84 (d, 1H), 7.99 (d, 1H), 8.44 (d, 1H), 8.55 (d, 1H), 10.69 (br. s., 1H). |
| 2-12-88 SM = 1-35-3 N,N- Diisopro- pylethyl- amine as base | 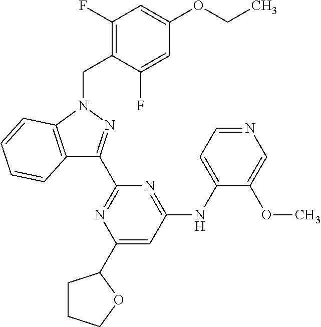 [SMILES: CCOc1cc(F)c(Cn2nc(-c3nc(Nc4ccncc4OC)cc(C4CCCO4)n3)c3ccccc32)c(F)c1] | 2-[1-(4-ethoxy-2,6- difluorobenzyl)-1H- indazol-3-yl]-N-(3- methoxypyridin-4-yl)-6- [(2rac)-tetrahydrofuran- 2-yl]pyrimidin-4-amine | ¹H-NMR (400 MHz, DMSO-d₆): δ [ppm] = 1.29 (t, 3H), 1.97 (m, 3H), 2.46 (m, 1H), 3.92 (m, 1H), 3.98 (s, 3H), 4.02 (m, 1H), 4.04 (q, 2H), 4.93 (m, 1H), 5.72 (s, 2H), 6.80 (m, 2H), 7.29 (dd, 1H), 7.31 (s, 1H), 7.50 (dd, 1H), 7.87 (d, 1H), 8.12 (d, 1H), 8.30 (s, 1H), 8.49 (d, 1H), 8.89 (d, 1H), 9.29 (br. s., 1H). |
| 2-12-89 SM = 1-35-4 N,N- Diisopro- pylethyl- amine as base | 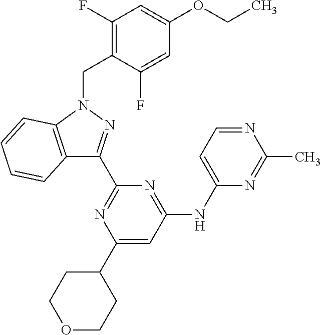 [SMILES: CCOc1cc(F)c(Cn2nc(-c3nc(Nc4ccnc(C)n4)cc(C4CCOCC4)n3)c3ccccc32)c(F)c1] | 2-[1-(4-ethoxy-2,6- difluorobenzyl)-1H- indazol-3-yl]-N-(2- methylpyrimidin-4-yl)-6- (tetrahydro-2H-pyran-4- yl)pyrimidin-4-amine | ¹H-NMR (400 MHz, DMSO-d₆): δ [ppm] = 1.29 (t, 3H), 1.84 (dddd, 2H), 1.90 (br. d., 2H), 2.56 (s, 3H), 2.97 (m, 1H), 3.52 (ddd, 2H), 4.02 (m, 2H), 4.03 (q, 2H), 5.72 (s, 2H), 6.80 (m, 2H), 7.34 (dd, 1H), 7.51 (dd, 1H), 7.55 (s, 1H), 7.85 (d, 1H), 8.00 (d, 1H), 8.44 (d, 1H), 8.59 (d, 1H), 10.67 (br. s., 1H). |
| 2-12-90 SM = 1-35-4 N,N- Diisopro- pylethyl- amine as base | 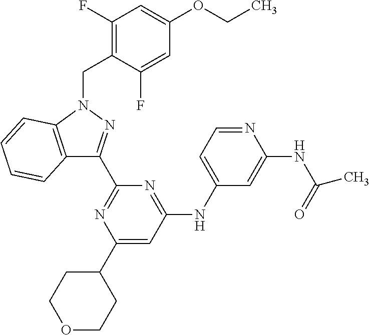 [SMILES: CCOc1cc(F)c(Cn2nc(-c3nc(Nc4ccnc(NC(C)=O)c4)cc(C4CCOCC4)n3)c3ccccc32)c(F)c1] | N-[4-({2-[1-(4-ethoxy- 2,6-difluorobenzyl)-1H- indazol-3-yl]-6- (tetrahydro-2H-pyran-4- yl)pyrimidin-4- yl}amino)pyridin-2- yl]acetamide | ¹H-NMR (400 MHz, DMSO-d₆): δ [ppm] = 1.28 (t, 3H), 1.81 (dddd, 2H), 1.89 (br. d., 2H), 2.10 (s, 3H), 2.92 (m, 1H), 3.51 (ddd, 2H), 4.00 (m, 2H), 4.03 (q, 2H), 5.72 (s, 2H), 6.73 (s, 1H), 6.80 (m, 2H), 7.32 (dd, 1H), 7.51 (dd, 1H), 7.86 (d, 1H), 7.98 (d, 1H), 8.13 (d, 1H), 8.46 (dd, 1H), 8.55 (d, 1H), 10.16 (br. s., 1H), 10.37 (br. s., 1H). |
| 2-12-91 SM = 1-35-4 N,N- Diisopro- pylethyl- amine as base | 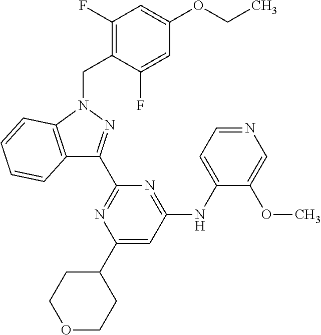 [SMILES: CCOc1cc(F)c(Cn2nc(-c3nc(Nc4ccncc4OC)cc(C4CCOCC4)n3)c3ccccc32)c(F)c1] | 2-[1-(4-ethoxy-2,6- difluorobenzyl)-1H- indazol-3-yl]-N-(3- methoxypyridin-4-yl)-6- (tetrahydro-2H-pyran-4- yl)pyrimidin-4-amine | ¹H-NMR (400 MHz, DMSO-d₆): δ [ppm] = 1.29 (t, 3H), 1.80 (dddd, 2H), 1.91 (br. d., 2H), 2.91 (m, 1H), 3.51 (ddd, 2H), 3.99 (s, 3H), 4.00 (m, 2H), 4.03 (q, 2H), 5.72 (s, 2H), 6.81 (m, 2H), 7.14 (s, 1H), 7.32 (dd, 1H), 7.51 (dd, 1H), 7.87 (d, 1H), 8.13 (d, 1H), 8.30 (s, 1H), 8.54 (d, 1H), 8.93 (d, 1H), 9.18 (br. s., 1H). |
| 2-12-92 SM = 1-35-4 N,N- Diisopro- pylethyl- amine as base | 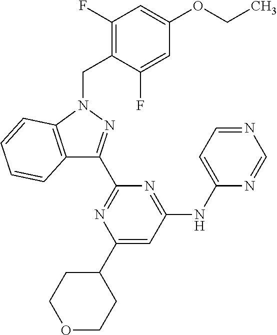 [SMILES: CCOc1cc(F)c(Cn2nc(-c3nc(Nc4ccncn4)cc(C4CCOCC4)n3)c3ccccc32)c(F)c1] | 2-[1-(4-ethoxy-2,6- difluorobenzyl)-1H- indazol-3-yl]-N- (pyrimidin-4-yl)-6- (tetrahydro-2H-pyran-4- yl)pyrimidin-4-amine | ¹H-NMR (400 MHz, DMSO-d₆): δ [ppm] = 1.29 (t, 3H), 1.87 (m, 4H), 2.99 (m, 1H), 3.52 (ddd, 2H), 4.01 (m, 2H), 4.04 (q, 2H), 5.73 (s, 2H), 6.81 (m, 2H), 7.35 (dd, 1H), 7.52 (dd, 1H), 7.62 (s, 1H), 7.85 (d, 1H), 8.09 (d, 1H), 8.53 (d, 1H), 8.59 (d, 1H), 8.86 (s, 1H), 10.75 (br. s., 1H). |
| 2-12-93 SM = 1-35-5 N,N- Diisopro- pylethyl- amine as base | 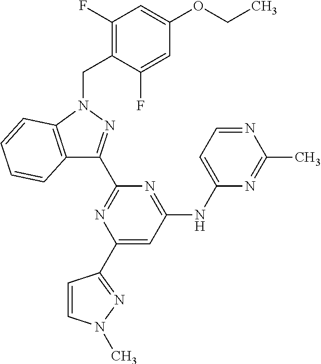 [SMILES: CCOc1cc(F)c(Cn2nc(-c3nc(Nc4ccnc(C)n4)cc(-c4ccn(C)n4)n3)c3ccccc32)c(F)c1] | 2-[1-(4-ethoxy-2,6- difluorobenzyl)-1H- indazol-3-yl]-6-(1- methyl-1H-pyrazol-3- yl)-N-(2- methylpyrimidin-4- yl)pyrimidin-4-amine | ¹H-NMR (400 MHz, DMSO-d₆): δ [ppm] = 1.29 (t, 3H), 2.57 (s, 3H), 3.99 (s, 3H), 4.04 (q, 2H), 5.74 (s, 2H), 6.82 (m, 2H), 7.04 (d, 1H), 7.37 (dd, 1H), 7.53 (dd, 1H), 7.88 (d, 1H), 7.91 (d, 1H), 8.04 (s, 1H), 8.18 (d, 1H), 8.46 (d, 1H), 8.66 (d, 1H), 10.73 (br. s., 1H). |
| 2-12-94 SM = 1-35-5 N,N- Diisopro- pylethyl- amine as base | 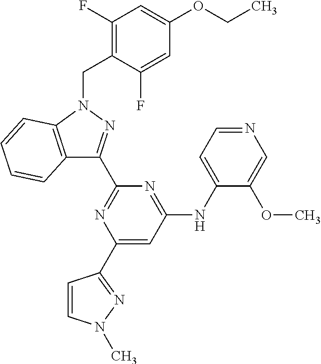 [SMILES: CCOc1cc(F)c(Cn2nc(-c3nc(Nc4ccncc4OC)cc(-c4ccn(C)n4)n3)c3ccccc32)c(F)c1] | 2-[1-(4-ethoxy-2,6- difluorobenzyl)-1H- indazol-3-yl]-N-(3- methoxypyridin-4-yl)-6- (1-methyl-1H-pyrazol-3- yl)pyrimidin-4-amine | ¹H-NMR (400 MHz, DMSO-d₆): δ [ppm] = 1.28 (t, 3H), 3.98 (s, 3H), 3.98 (s, 3H), 4.04 (q, 2H), 5.73 (s, 2H), 6.81 (m, 2H), 7.02 (d, 1H), 7.35 (dd, 1H), 7.53 (dd, 1H), 7.78 (s, 1H), 7.89 (d, 1H), 7.90 (d, 1H), 8.14 (d, 1H), 8.30 (s, 1H), 8.65 (d, 1H), 8.99 (d, 1H), 9.33 (br. s., 1H). |
| 2-12-95 SM = 1-35-6 N,N- Diisopro- pylethyl- amine as base | 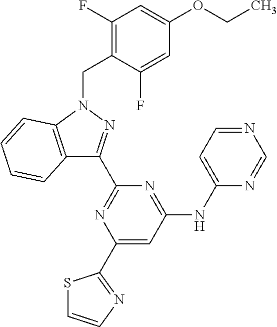 [SMILES: CCOc1cc(F)c(Cn2nc(-c3nc(Nc4ccncn4)cc(-c4nccs4)n3)c3ccccc32)c(F)c1] | 2-[1-(4-ethoxy-2,6- difluorobenzyl)-1H- indazol-3-yl]-N- (pyrimidin-4-yl)-6-(1,3- thiazol-2-yl)pyrimidin-4- amine | ¹H-NMR (400 MHz, DMSO-d₆): δ [ppm] = 1.29 (t, 3H), 4.04 (q, 2H), 5.77 (s, 2H), 6.82 (m, 2H), 7.41 (dd, 1H), 7.56 (dd, 1H), 7.90 (d, 1H), 8.08 (d, 1H), 8.15 (m, 1H), 8.16 (d, 1H), 8.43 (s, 1H), 8.59 (d, 1H), 8.70 (d, 1H), 8.92 (s, 1H), 11.06 (br. s., 1H). |
| 2-12-96 SM = 1-35-6 N,N- Diisopro- pylethyl- amine as base | 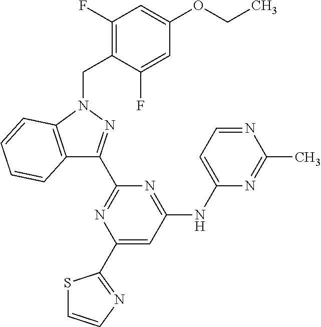 [SMILES: CCOc1cc(F)c(Cn2nc(-c3nc(Nc4ccnc(C)n4)cc(-c4nccs4)n3)c3ccccc32)c(F)c1] | 2-[1-(4-ethoxy-2,6- difluorobenzyl)-1H- indazol-3-yl]-N-(2- methylpyrimidin-4-yl)- 6-(1,3-thiazol-2- yl)pyrimidin-4-amine | ¹H-NMR (400 MHz, DMSO-d₆): δ [ppm] = 1.29 (t, 3H), 2.59 (s, 3H), 4.04 (q, 2H), 5.76 (s, 2H), 6.82 (m, 2H), 7.40 (dd, 1H), 7.56 (dd, 1H), 7.91 (d, 1H), 8.07 (d, 1H), 8.17 (d, 1H), 8.16 (m, 1H), 8.27 (s, 1H), 8.49 (d, 1H), 8.69 (d, 1H), 11.00 (br. s., 1H). |
| 2-12-97 SM = 1-35-1 N,N- Diisopro- pylethyl- amine as base | 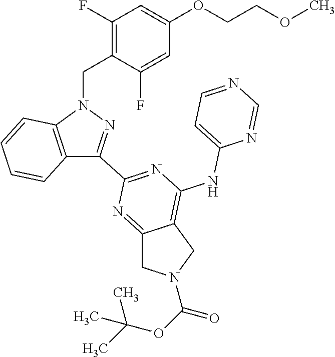 [SMILES: COCCOc1cc(F)c(Cn2nc(-c3nc4c(c(Nc5ccncn5)n3)CN(C(=O)OC(C)(C)C)C4)c3ccccc32)c(F)c1] | tert-butyl 2-{1-[2,6- difluoro-4-(2- methoxyethoxy)benzyl]- 1H-indazol-3-yl}-4- (pyrimidin-4-ylamino)- 5,7-dihydro-6H- pyrrolo[3,4- d]pyrimidine-6- carboxylate | ¹H-NMR (400 MHz, DMSO-d₆): δ [ppm] = 1.48/1.49 (s, 9H), 3.26 (s, 3H), 3.61 (m, 2H), 4.11 (m, 2H), 4.61/4.65 (br. s., 2H), 4.68/4.72 (br. s., 2H), 5.72 (s, 2H), 6.86 (m, 2H), 7.31/7.30 (m, 1H), 7.51 (dd, 1H), 7.88 (d, 1H), 8.49/8.51 (d, 1H), 8.60 (d, 1H), 8.65/8.69 (d, 1H), 8.89 (s, 1H), 10.54/10.60 (br. s., 1H). |
| 2-12-98 SM = 1-35-1 | 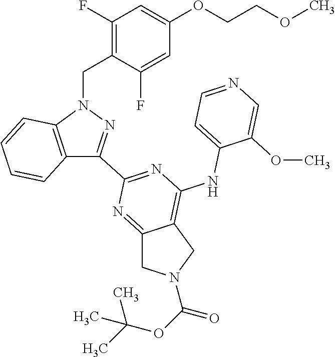 [SMILES: COCCOc1cc(F)c(Cn2nc(-c3nc4c(c(Nc5ccncc5OC)n3)CN(C(=O)OC(C)(C)C)C4)c3ccccc32)c(F)c1] | tert-butyl 2-{1-[2,6- difluoro-4-(2- methoxyethoxy)benzyl]- 1H-indazol-3-yl}-4-[(3- methoxypyridin-4- yl)amino]-5,7-dihydro- 6H-pyrrolo[3,4- d]pyrimidine-6- carboxylate | ¹H-NMR (400 MHz, DMSO-d₆): δ [ppm] = 1.48/1.49 (s, 9H), 3.26 (s, 3H), 3.61 (m, 2H), 3.93/3.95 (s, 3H), 4.11 (m, 2H), 4.59/4.62 (br. s., 2H), 4.65/4.69 (br. s., 2H), 5.69 (s, 2H), 6.82 (m, 2H), 7.17/7.20 (m, 1H), 7.46/7.48 (m, 1H), 7.82/7.84 (d, 1H), 8.19 (s, 1H), 8.26/8.36 (d, 1H), 8.40 (m, 2H), 8.62/8.77 (br. s., 1H). |
| 2-12-99 SM = 1-20-17 | 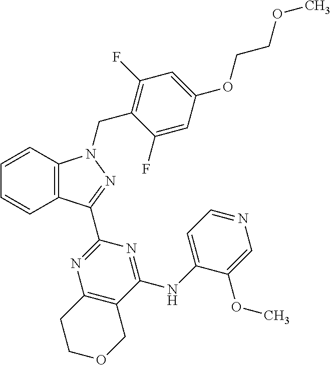 [SMILES: COCCOc1cc(F)c(Cn2nc(-c3nc4c(c(Nc5ccncc5OC)n3)COCC4)c3ccccc32)c(F)c1] | 2-{1-[2,6-difluoro-4-(2- methoxyethoxy)benzyl]- 1H-indazol-3-yl}-N-(3- methoxypyridin-4-yl)- 7,8-dihydro-5H- pyrano[4,3-d]pyrimidin- 4-amin | ¹H-NMR (400 MHz, DMSO-d₆): δ [ppm] = 3.11 (t, 2H), 3.44 (s, 3H), 3.66-3.76 (m, 2H), 4.00- 4.10 (m, 5H), 4.13 (t, 2H), 4.80 (s, 2H), 5.75 (s, 2H), 6.47-6.59 (m, 2H), 6.97 (s, 1H), 7.28 (t, 1H), 7.41- 7.49 (m, 1H), 7.61 (d, 1H), 8.26 (s, 1H), 8.34 (d, 1H), 8.60 (d, 1H), 8.94 (d, 1H) |
| 2-12-100 SM = 1-20-17 | 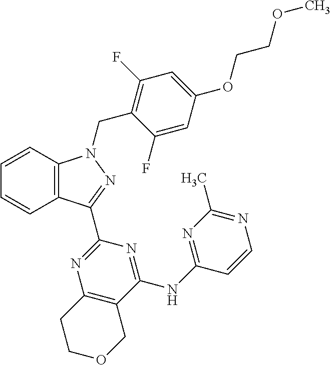 [SMILES: COCCOc1cc(F)c(Cn2nc(-c3nc4c(c(Nc5ccnc(C)n5)n3)COCC4)c3ccccc32)c(F)c1] | 2-{1-[2,6-difluoro-4-(2- methoxyethoxy)benzyl]- 1H-indazol-3-yl}-N-(2- methylpyrimidin-4-yl)- 7,8-dihydro-5H- pyrano[4,3-d]pyrimidin- 4-amine | ¹H-NMR (400 MHz, DMSO-d₆): δ [ppm] = 2.56 (s, 3H), 2.95 (t, 2H), 3.27 (s, 3H), 3.58-3.65 (m, 2H), 4.00 (t, 2H), 4.09- 4.15 (m, 2H), 4.79 (s, 2H), 5.72 (s, 2H), 6.80-6.90 (m, 2H), 7.29 (t, 1H), 7.45- 7.54 (m, 1H), 7.86 (d, 1H), 8.39 (d, 1H), 8.46-8.53 (m, 2H), 9.59 (s, 1H). |

### Example 2-13-1

### Preparation of 3-{2-[1-(4-ethoxy-2,6-difluorobenzyl)-1H-indazol-3-yl]-4-(pyridin-4-ylamino)pyrimidin-5-yl}propan-1-ol

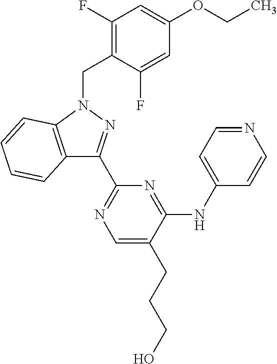 [SMILES: CCOc1cc(F)c(Cn2nc(-c3ncc(CCCO)c(Nc4ccncc4)n3)c3ccccc32)c(F)c1]

125 mg 5-(3-{[tert-butyl(dimethyl)silyl]oxy}propyl)-2-[1-(4-ethoxy-2,6-difluorobenzyl)-1H-indazol-3-yl]-N-(pyridin-4-yl)pyrimidin-4-amine 2-2-22 (0.20 mmol, 1.0 eq.) were dissolved in 3 mL THF. At 0° C. bath temperature, 0.8 mL Tetrabutylammonium fluoride (1N in THF, 0.8 mmol, 4.0 eq.) were added. It was stirred at rt for 4 hours. Under ice bath cooling, half concentrated sodium hydrogen carbonate solution was added. The mixture was extracted four times with dichloromethane/2-propanole 4:1, and filtered through a water resistant filter. The filtrate was concentrated under reduced pressure and crystallized from methanol to provide 70 mg of the target compound in 96% purity: 69%.

¹H-NMR (400 MHz, DMSO-d₆): δ [ppm]=1.28 (t, 3H), 1.70-1.84 (m, 2H), 2.77 (t, 2H), 3.49 (t, 2H), 4.03 (q, 2H), 4.84 (br. s., 1H), 5.69 (s, 2H), 6.71-6.87 (m, 2H), 7.25 (t, 1H), 7.49 (ddd, 1H), 7.85 (d, 1H), 7.93-8.11 (m, 2H), 8.34-8.49 (m, 4H), 8.99 (br. s., 1H).

### Example 2-14-1

### Preparation 2-[1-(4-ethoxy-2,6-difluorobenzyl)-1H-indazol-3-yl]-N-(3-fluoropyridin-4-yl)-6,7-dihydro-5H-cyclopenta[d]pyrimidin-4-amine

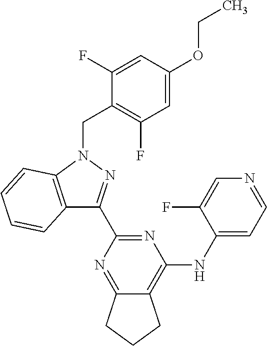 [SMILES: CCOc1cc(F)c(Cn2nc(-c3nc4c(c(Nc5ccncc5F)n3)CCC4)c3ccccc32)c(F)c1]

150 mg (0.34 mmol, 1.0 eq.) 3-(4-chloro-6,7-dihydro-5H-cyclopenta[d]pyrimidin-2-yl)-1-(4-ethoxy-2,6-difluorobenzyl)-1H-indazole 1-13-1, 38 mg (0.34 mmol, 1.0 eq.) 4-amino-3-fluoropyridine, 166 mg (0.51 mmol, 1.5 eq.) caesium carbonate, 4 mg (0.02 mmol, 0.05 eq.) palladium acetate and 12 mg (0.02 mmol, 0.056 eq.) (rac)-2,2′-bis(diphenylphosphino)-1,1′-binaphthyl were dissolved under inert atmosphere in 2.0 mL N,N-dimethylformamide. The suspension was heated to 100° C. overnight, cooled to room temperature and extracted with dichloromethane and water. The organic layer was filtered through a silicon filter and concentrated under reduced pressure. The crude product was purified by HPLC to yield 42 mg (22% yield) of 2-[1-(4-ethoxy-2,6-difluorobenzyl)-1H-indazol-3-yl]-N-(3-fluoropyridin-4-yl)-6,7-dihydro-5H-cyclopenta[d]pyrimidin-4-amine.

¹H NMR (400 MHz, DMSO-d₆) δ ppm 1.28 (t, 3H) 2.10 (quin, 2H) 2.82-3.07 (m, 4H) 4.03 (q, 2H) 5.67 (s, 2H) 6.76 (s, 1H) 6.78 (s, 1H) 7.16 (dd, 1H) 7.45 (ddd, 1H) 7.79 (d, 1H) 8.22-8.40 (m, 3H) 8.54 (d, 1H) 8.92 (s, 1H).

The following compounds were prepared according to the same procedure from the indicated starting materials (SM=starting material):

| | | | |
|---|---|---|---|
| 2-14-2 SM = 1-13-1 | 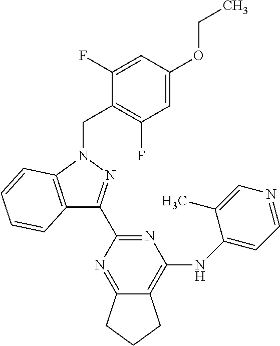 [SMILES: CCOc1cc(F)c(Cn2nc(-c3nc4c(c(Nc5ccncc5C)n3)CCC4)c3ccccc32)c(F)c1] | 2-[1-(4-ethoxy-2,6- difluorobenzyl)-1H- indazol-3-yl]-N-(3- methylpyridin-4-yl)-6,7- dihydro-5H- cyclopenta[d]pyrimidin- 4-amine | ¹H NMR (400 MHz, DMSO-d₆) δ ppm 1.28 (t, 3 H) 2.10 (quin, 2 H) 2.24 (s, 3 H) 2.82-3.01 (m, 4 H) 4.03 (q, 2 H) 5.64 (s, 2 H) 6.75 (s, 1 H) 6.77 (s, 1 H) 7.09 (t, 1 H) 7.42 (ddd, 1 H) 7.69-7.85 (m, 2H) 8.17 (d, 1 H) 8.28-8.50 (m, 3 H). |
| 2-14-3 SM = 1-13-1 | 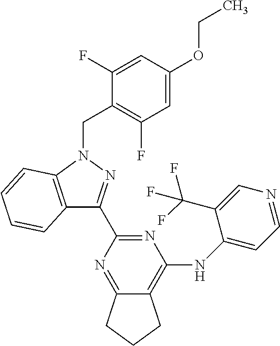 [SMILES: CCOc1cc(F)c(Cn2nc(-c3nc4c(c(Nc5ccncc5C(F)(F)F)n3)CCC4)c3ccccc32)c(F)c1] | 2-[1-(4-ethoxy-2,6- difluorobenzyl)-1H- indazol-3-yl]-N-[3- (trifluoromethyl)pyridin- 4-yl]-6,7-dihydro-5H- cyclopenta[d]pyrimidin- 4-amine | ¹H NMR (400 MHz, DMSO-d₆) δ ppm 1.30 (t, 3 H) 2.00-2.22 (m, 2 H) 2.84-2.88 (m, 2H) 2.96-3.00 (m, 2 H) 4.05 (q, 2 H) 5.67 (s, 2 H) 6.77 (s, 1 H) 6.79 (s, 1 H) 7.11 (t, 1 H) 7.44 (t, 1 H) 7.78 (d, 1 H) 8.05-8.25 (m, 2 H) 8.47 (br. s., 1 H) 8.72 (br. s., 1 H) 8.87 (br. s., 1 H). |
| 2-14-4 SM = 1-13-1 | 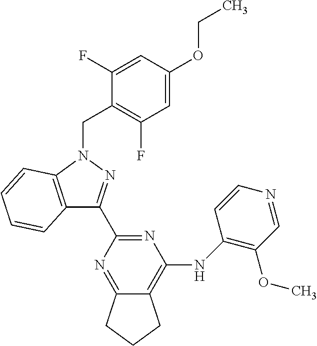 [SMILES: CCOc1cc(F)c(Cn2nc(-c3nc4c(c(Nc5ccncc5OC)n3)CCC4)c3ccccc32)c(F)c1] | 2-[1-(4-ethoxy-2,6- difluorobenzyl)-1H- indazol-3-yl]-N-(3- methoxypyridin-4-yl)- 6,7-dihydro-5H- cyclopenta[d]pyrimidin- 4-amine | ¹H NMR (400 MHz, DMSO-d₆) δ ppm 1.28 (t, 3 H) 2.05-2.19 (m, 2 H) 2.85-3.04 (m, 4 H) 3.97 (s, 3 H) 4.04 (q, 2 H) 5.68 (s, 2 H) 6.77 (s, 1 H) 6.80 (s, 1 H) 7.22 (t, 1 H) 7.47 (t, 1 H) 7.83 (d, 1 H) 7.90 (s, 1 H) 8.15 (d, 1 H) 8.34 (s, 1 H) 8.41 (d, 1 H) 8.60 (d, 1 H). |
| 2-14-5 SM = 1-13-1 | 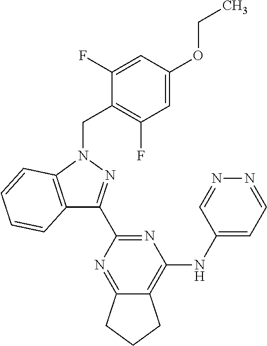 [SMILES: CCOc1cc(F)c(Cn2nc(-c3nc4c(c(Nc5ccnnc5)n3)CCC4)c3ccccc32)c(F)c1] | 2-[1-(4-ethoxy-2,6- difluorobenzyl)-1H- indazol-3-yl]-N- (pyridazin-4-yl)-6,7- dihydro-5H- cyclopenta[d]pyrimidin- 4-amine | ¹H NMR (400 MHz, DMSO-d₆) δ ppm 1.28 (t, 3 H) 2.10 (quin, 2 H) 2.24 (s, 3 H) 2.82-3.01 (m, 4 H) 4.03 (q, 2 H) 5.64 (s, 2 H) 6.75 (s, 1 H) 6.77 (s, 1 H) 7.09 (t, 1 H) 7.42 (ddd, 1 H) 7.69-7.85 (m, 2 H) 8.17 (d, 1 H) 8.28-8.50 (m, 3 H). |
| 2-14-6 SM = 1-13-1 | 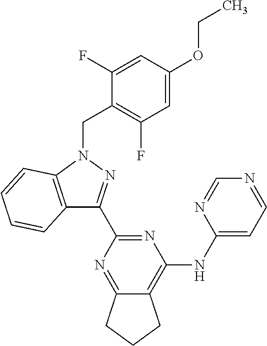 [SMILES: CCOc1cc(F)c(Cn2nc(-c3nc4c(c(Nc5ccncn5)n3)CCC4)c3ccccc32)c(F)c1] | 2-[1-(4-ethoxy-2,6- difluorobenzyl)-1H- indazol-3-yl]-N- (pyrimidin-4-yl)-6,7- dihydro-5H- cyclopenta[d]pyrimidin- 4-amine | ¹H NMR (300 MHz, DMSO-d₆) δ ppm 1.24-1.38 (m, 3 H) 1.90-2.15 (m, 2 H) 2.84-3.10 (m, 4 H) 4.03 (q, 2 H) 5.69 (s, 2 H) 6.79 (s, 1 H) 6.82 (s, 1 H) 7.26 (t, 1 H) 7.48 (t, 1 H) 7.84 (d, 1 H) 8.31-8.63 (m, 3 H) 8.80 (s, 1 H) 10.07 (br. s., 1 H). |
| 2-14-7 SM = 1-13-1 | 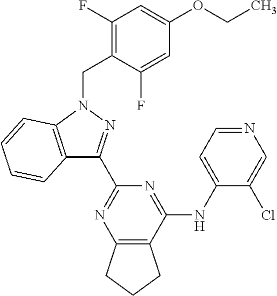 [SMILES: CCOc1cc(F)c(Cn2nc(-c3nc4c(c(Nc5ccncc5Cl)n3)CCC4)c3ccccc32)c(F)c1] | N-(3-chloropyridin-4-yl)- 2-[1-(4-ethoxy-2,6- difluorobenzyl)-1H- indazol-3-yl]-6,7- dihydro-5H- cyclopenta[d]pyrimidin- 4-amine | ¹H-NMR (400 MHz, DMSO-d₆): δ [ppm] = 1.22-1.40 (m, 3 H) 2.14 (quin, 2 H) 2.87-3.09 (m, 4 H) 4.05 (q, 2 H) 5.69 (s, 2 H) 6.78 (s, 1 H) 6.81 (s, 1 H) 7.10 (t, 1 H) 7.33-7.42 (m, 2 H) 7.42-7.54 (m, 1 H) 7.82 (d, 1 H) 8.27 (d, 1 H) 8.36-8.41 (m, 1 H) 8.41-8.49 (m, 1 H). |
| 2-14-8 SM = 1-13-1 | 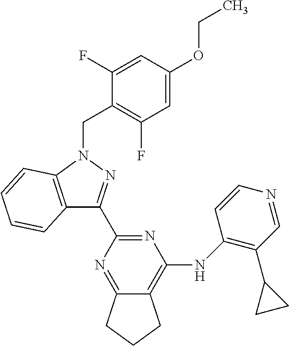 [SMILES: CCOc1cc(F)c(Cn2nc(-c3nc4c(c(Nc5ccncc5C5CC5)n3)CCC4)c3ccccc32)c(F)c1] | N-(3-cyclopropylpyridin- 4-yl)-2-[1-(4-ethoxy-2,6- difluorobenzyl)-1H- indazol-3-yl]-6,7- dihydro-5H- cyclopenta[d]pyrimidin- 4-amine | ¹H-NMR (400 MHz, DMSO-d₆): δ [ppm] = 0.59-0.75 (m, 2 H) 0.84-0.99 (m, 2 H) 1.28 (t, 3 H) 1.93-2.07 (m, 1 H) 2.11-2.16 (m, 2 H) 2.92-3.00 (m, 4 H) 4.03 (q, 2 H) 5.67 (s, 2 H) 6.76 (s, 1 H) 6.78 (s, 1 H) 7.17 (t, 1 H) 7.45 (td, 1 H) 7.79 (d, 1 H) 8.16 (s, 1 H) 8.22-8.43 (m, 4 H). |
| 2-14-9 SM = 1-13-1, methyl 4- aminopyridine- 3-carboxylate | 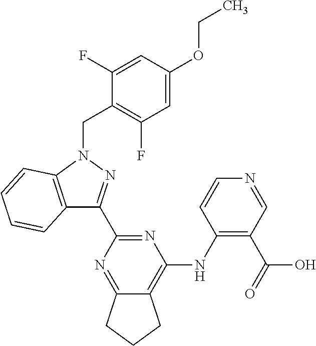 [SMILES: CCOc1cc(F)c(Cn2nc(-c3nc4c(c(Nc5ccncc5C(=O)O)n3)CCC4)c3ccccc32)c(F)c1] | 4-({2-[1-(4-ethoxy-2,6- difluorobenzyl)-1H- indazol-3-yl]-6,7- dihydro-5H- cyclopenta[d]pyrimidin- 4-yl}amino)pyridine-3- carboxylic acid | ¹H-NMR (400 MHz, CDCl₃): δ [ppm] = 1.43 (t, 3 H) 2.38-2.42 (m, 2 H) 3.26-3.30 (m, 4 H) 4.13 (q, 2 H) 5.59 (s, 2 H) 6.63 (s, 1 H) 6.65 (s, 1 H) 7.37-7.47 (m, 1 H) 7.55 (t, 1 H) 7.73 (s, 1 H) 7.85 (d, 1 H) 8.21 (s, 1 H) 8.64 (d, 1 H) 9.31 (s, 1 H) |
| 2-14-10 SM = 1-20-5 | 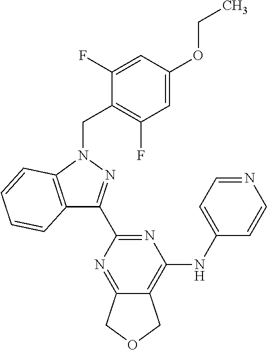 [SMILES: CCOc1cc(F)c(Cn2nc(-c3nc4c(c(Nc5ccncc5)n3)COC4)c3ccccc32)c(F)c1] | 2-[1-(4-ethoxy-2,6- difluorobenzyl)-1H- indazol-3-yl]-N-(pyridin- 4-yl)-5,7- dihydrofuro[3,4- d]pyrimidin-4-amine | ¹H-NMR (400 MHz, DMSO-d₆): δ [ppm] = 1.28 (t, 3 H) 4.03 (q, 2 H) 5.02 (s, 2 H) 5.14 (s, 2 H) 5.71 (s, 2 H) 6.78 (s, 1 H) 6.82 (s, 1 H) 7.28 (t, 1 H) 7.50 (t, 1 H) 7.88 (d, 1 H) 8.07-8.10 (m, 2 H) 8.44-8.47 (m, 3 H) 9.71 (s, 1 H). |

### Example 2-15-1

### Preparation of 2-[1-(4-ethoxy-2,6-difluorobenzyl)-1H-indazol-3-yl]-6-ethyl-N-(pyridin-4-yl)-5,6,7,8-tetrahydropyrido[4,3-d]pyrimidin-4-amine

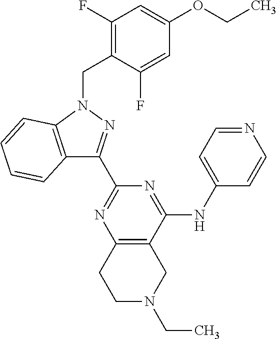 [SMILES: CCOc1cc(F)c(Cn2nc(-c3nc4c(c(Nc5ccncc5)n3)CN(CC)CC4)c3ccccc32)c(F)c1]

115 mg (0.192 mmol) 2-[1-(4-ethoxy-2,6-difluorobenzyl)-1H-indazol-3-yl]-6-ethyl-5,6,7,8-tetrahydropyrido[4,3-d]pyrimidin-4-yl trifluoromethanesulfonate 1-18-1, 20 mg (0.212 mmol) 4-aminopyridine, 4.3 mg (0.02 mmol) palladium(II) acetate, 16.7 mg (0.03 mmol) 4,5-bis(diphenylphosphino)-9,9-dimethylxanthene and 188 mg (0.02 mmol) caesium carbonate were placed in a microwave vial. Under inert atmosphere 2.5 mL dioxane were added and the reaction mixture was stirred at an environmental temperature of 115° C. overnight. The reaction mixture was diluted with dichloromethane/isopropanol (4/1) and passed through a Whatman filter. The resulting organic layer was concentrated under reduced pressure and purified by HPLC to yield 20 mg (17% yield of theory) of the title compound.

¹H-NMR (400 MHz, DMSO-d₆): δ [ppm]=1.19 (t, 3H), 1.30 (t, 3H), 2.64-2.71 (m, 2H), 2.75-2.85 (m, 2H), 2.88-2.99 (m, 2H), 3.59 (s, 2H), 4.05 (q, 2H), 5.72 (s, 2H), 6.74-6.86 (m, 2H), 7.25 (t, 1H), 7.49 (ddd, 1H), 7.85 (d, 1H), 7.97-8.08 (m, 2H), 8.36-8.44 (m, 2H), 8.48 (d, 1H), 8.75 (s, 1H).

The following compounds were prepared according to the same procedure from the indicated starting materials (SM=starting material):

| | | | |
|---|---|---|---|
| 2-15-2 SM = 1-18-1 | 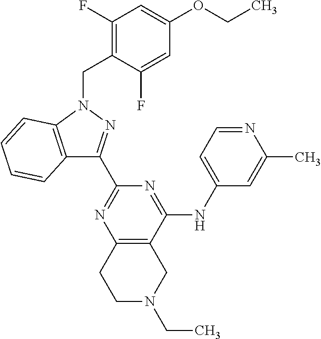 [SMILES: CCOc1cc(F)c(Cn2nc(-c3nc4c(c(Nc5ccnc(C)c5)n3)CN(CC)CC4)c3ccccc32)c(F)c1] | 2-[1-(4-ethoxy-2,6- difluorobenzyl)-1H- indazol-3-yl]-6-ethyl-N- (2-methylpyridin-4-yl)- 5,6,7,8- tetrahydropyrido[4,3- d]pyrimidin-4-amine | ¹H-NMR (400MHz, DMSO-d₆): δ [ppm] = 1.17 (t, 3H), 1.27 (t, 3H), 2.43 (s, 3H), 2.57-2.70 (m, 2H), 2.77 (t, 2H), 2.90 (t, 2H), 3.56 (s, 2H), 4.02 (q, 2H), 5.70 (s, 2H), 6.69- 6.82 (m, 2H), 7.23 (t, 1H), 7.47 (ddd, 1H), 7.76- 7.85 (m, 2H), 7.89 (d, 1H), 8.28 (d, 1H), 8.48 (d, 1H), 8.63 (s, 1H). |
| 2-15-3 SM = 1-18-2 | 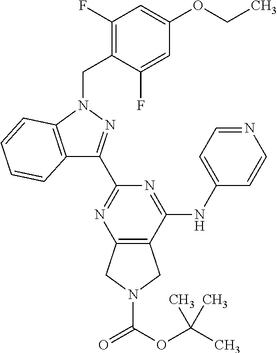 [SMILES: CCOc1cc(F)c(Cn2nc(-c3nc4c(c(Nc5ccncc5)n3)CN(C(=O)OC(C)(C)C)C4)c3ccccc32)c(F)c1] | tert-butyl 2-[1-(4- ethoxy-2,6- difluorobenzyl)-1H- indazol-3-yl]-4-(pyridin- 4-ylamino)-5,7-dihydro- 6H-pyrrolo[3,4- d]pyrimidine-6- carboxylate | ¹H-NMR (400MHz, DMSO-d₆): δ [ppm] = 1.28 (t, 3H), 1.45-1.56 (m, 9H), 4.05 (q, 2H), 4.56- 4.75 (m, 4H), 5.66-5.78 (m, 2H), 6.82 (d, 2H), 7.30 (t, 1H), 7.52 (t, 1H), 7.89 (d, 1H), 8.19 (d, 1H), 8.15 (d, 1H), 8.42- 8.56 (m, 3H), 9.77 (br. s., 1H). |
| 2-15-4 SM = 1-18-2 | 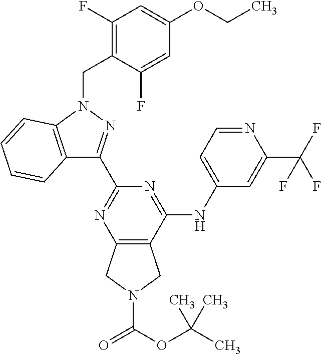 [SMILES: CCOc1cc(F)c(Cn2nc(-c3nc4c(c(Nc5ccnc(C(F)(F)F)c5)n3)CN(C(=O)OC(C)(C)C)C4)c3ccccc32)c(F)c1] | tert-butyl 2-[1-(4- ethoxy-2,6- difluorobenzyl)-1H- indazol-3-yl]-4-{[2- (trifluoromethyl)pyridin- 4-yl]amino}-5,7-dihydro- 6H-pyrrolo[3,4- d]pyrimidine-6- carboxylate | ¹H-NMR (400MHz, DMSO-d₆): δ [ppm] = 1.28 (t, 3H), 1.51 (s, 9H), 4.04 (q, 2H), 4.57-4.76 (m, 4H), 5.74 (s, 2H), 6.80 (d, 2H), 7.28 (t, 1H), 7.52 (td, 1H), 7.85 (d, 1H), 8.37-8.55 (m, 3H), 8.61 (d, 1H), 9.92 (d, 1H). |
| 2-15-5 SM = 1-18-3 | 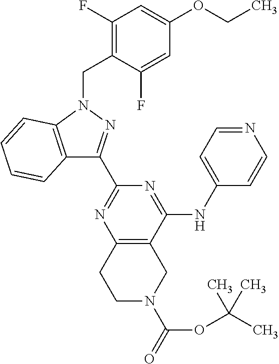 [SMILES: CCOc1cc(F)c(Cn2nc(-c3nc4c(c(Nc5ccncc5)n3)CN(C(=O)OC(C)(C)C)CC4)c3ccccc32)c(F)c1] | tert-butyl 2-[1-(4- ethoxy-2,6- difluorobenzyl)-1H- indazol-3-yl]-4-(pyridin- 4-ylamino)-7,8- dihydropyrido[4,3- d]pyrimidine-6(5H)- carboxylate | ¹H-NMR (400MHz, DMSO-d₆): δ [ppm] = 1.30 (t, 3H), 1.47 (s, 9H), 2.92 (t, 2H), 3.73 (t, 2H), 4.05 (q, 2H), 4.59 (s, 2H), 5.71 (s, 2H), 6.80 (d, 2H), 7.26 (t, 1H), 7.41-7.56 (m, 1H), 7.85 (d, 1H), 8.06 (d, 2H), 8.43 (d, 2H), 8.47 (d, 1H), 8.96 (br. s., 1H). |
| 2-15-6 SM = 1-18-3 | 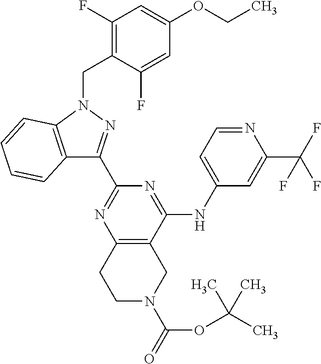 [SMILES: CCOc1cc(F)c(Cn2nc(-c3nc4c(c(Nc5ccnc(C(F)(F)F)c5)n3)CN(C(=O)OC(C)(C)C)CC4)c3ccccc32)c(F)c1] | tert-butyl 2-[1-(4- ethoxy-2,6- difluorobenzyl)-1H- indazol-3-yl]-4-{[2- (trifluoromethyl)pyridin- 4-yl]amino}-7,8- dihydropyrido[4,3- d]pyrimidine-6(5H)- carboxylate | ¹H-NMR (400MHz, DMSO-d₆): δ [ppm] = 1.29 (t, 3H), 1.44-1.52 (m, 9H), 2.94 (t, 2H), 3.74 (t, 2H), 4.04 (q, 2H), 4.62 (s, 2H), 5.73 (s, 2H), 6.79 (d, 2H), 7.25 (t, 1H), 7.42- 7.56 (m, 1H), 7.83 (d, 1H), 8.42-8.55 (m, 3H), 8.60 (d, 1H), 9.33 (br. s., 1H). |
| 2-15-7 SM = 1-18-4 | 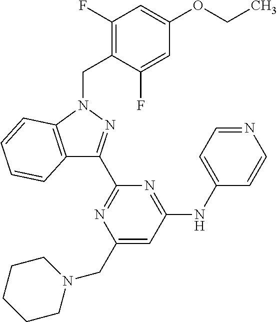 [SMILES: CCOc1cc(F)c(Cn2nc(-c3nc(CN4CCCCC4)cc(Nc4ccncc4)n3)c3ccccc32)c(F)c1] | 2-[1-(4-ethoxy-2,6- difluorobenzyl)-1H- indazol-3-yl]-6- (piperidin-1-ylmethyl)- N-(pyridin-4- yl)pyrimidin-4-amine | ¹H-NMR (400MHz, DMSO-d₆): δ [ppm] = 1.28 (t, 3H), 1.44 (d, 2H), 1.57 (d, 4H), 3.59 (s, 2H), 4.04 (q, 2H), 5.70 (s, 2H), 6.80 (d, 2H), 6.96 (s, 1H), 7.29 (t, 1H), 7.50 (t, 1H), 7.86 (d, 1H), 7.92 (d, 2H), 8.39 (d, 2H), 8.53 (d, 1H), 10.04 (s, 1H). 4H's not assigned |
| 2-15-8 SM = 1-18-5 | 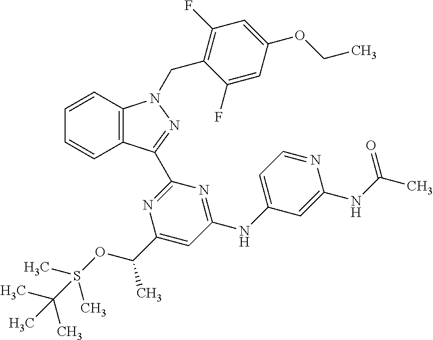 [SMILES: CCOc1cc(F)c(Cn2nc(-c3nc(Nc4ccnc(NC(C)=O)c4)cc([C@H](C)OS(C)(C)C(C)(C)C)n3)c3ccccc32)c(F)c1] | N-[4-({6-[(1S)-1-{[tert- butyl(dimethyl)silyl]oxy} ethyl]-2-[1-(4-ethoxy- 2,6-difluorobenzyl)-1H- indazol-3-yl]pyrimidin-4- yl}amino)pyridin-2- yl]acetamide | ¹H-NMR (400MHz, DMSO-d₆): δ [ppm] = 0.12 (s, 3H), 0.15 (s, 3H), 0.96 (s, 9H), 1.29 (t, 3H), 1.50 (d, 3H), 2.11 (s, 3H), 4.04 (q, 2H), 4.90 (q, 1H), 5.72 (s, 2H), 6.76-6.84 (m, 2H), 7.02 (s, 1H), 7.31 (t, 1H), 7.47-7.54 (m, 1H), 7.86 (d, 1H), 8.00 (s, 1H), 8.15 (d, 1H), 8.46-8.55 (m, 2H), 10.24 (br. s., 1H), 10.41 (br. s, 1H). |
| 2-15-9 SM = 1-18-6 | 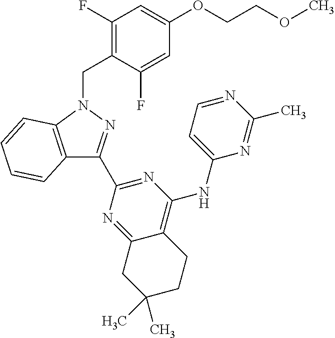 [SMILES: COCCOc1cc(F)c(Cn2nc(-c3nc4c(c(Nc5ccnc(C)n5)n3)CCC(C)(C)C4)c3ccccc32)c(F)c1] | 2-{1-[2,6-difluoro-4-(2- methoxyethoxy)benzyl]- 1H-indazol-3-yl}-7,7- dimethyl-N-(2- methylpyrimidin-4-yl)- 5,6,7,8- tetrahydroquinazolin-4- amine | ¹H-NMR (400 MHz, DMSO- d₆): δ [ppm] = 1.01 (s, 6H), 1.62 (m, 2H), 2.55 (s, 3H), 2.67 (br. s., 2H), 2.77 (m, 2H), 3.26 (s, 3H), 3.61 (m, 2H), 4.11 (m, 2H), 5.70 (s, 2H), 6.84 (m, 2H), 7.26 (dd, 1H), 7.49 (dd, 1H), 7.84 (d, 1H), 8.44 (d, 1H), 8.47 (d, 1H), 8.50 (d, 1H), 9.19 (br. s., 1H). |
| 2-15-10 SM = 1-18-6 | 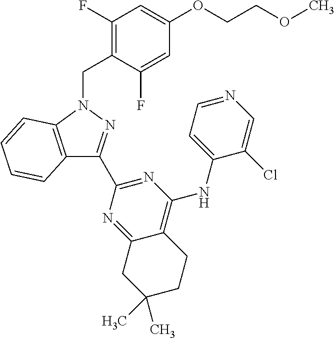 [SMILES: COCCOc1cc(F)c(Cn2nc(-c3nc4c(c(Nc5ccncc5Cl)n3)CCC(C)(C)C4)c3ccccc32)c(F)c1] | N-(3-chloropyridin-4-yl)- 2-{1-[2,6-difluoro-4-(2- methoxyethoxy)benzyl]- 1H-indazol-3-yl}-7,7- dimethyl-5,6,7,8- tetrahydroquinazolin-4- amine | ¹H-NMR (400 MHz, CDCl₃): δ [ppm] = 1.09 (s, 6H), 1.78 (m, 2H), 2.70 (m, 2H), 2.82 (br. s., 2H), 3.42 (s, 3H), 3.71 (m, 2H), 4.06 (m, 2H), 5.72 (s, 2H), 6.52 (m, 2H), 7.26 (dd, 1H), 7.38 (br. s., 1H), 7.43 (dd, 1H), 7.59 (d, 1H), 8.50 (d, 1H), 8.55 (s, 1H), 8.58 (d, 1H), 9.18 (d, 1H). |
| 2-15-11 SM = 1-18-6 | 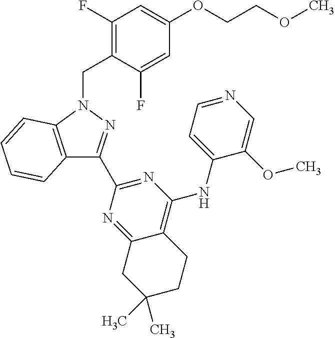 [SMILES: COCCOc1cc(F)c(Cn2nc(-c3nc4c(c(Nc5ccncc5OC)n3)CCC(C)(C)C4)c3ccccc32)c(F)c1] | 2-{1-[2,6-difluoro-4-(2- methoxyethoxy)benzyl]- 1H-indazol-3-yl}-N-(3- methoxypyridin-4-yl)- 7,7-dimethyl-5,6,7,8- tetrahydroquinazolin-4- amine | ¹H-NMR (400 MHz, CDCl₃): δ [ppm] = 1.08 (s, 6H), 1.76 (m, 2H), 2.66 (m, 2H), 2.80 (br. s., 2H), 3.42 (s, 3H), 3.71 (m, 2H), 4.06 (m, 2H), 4.07 (s, 3H), 5.72 (s, 2H), 6.52 (m, 2H), 7.25 (dd, 1H), 7.42 (dd, 1H), 7.45 (br. s., 1H), 7.58 (d, 1H), 8.23 (s, 1H), 8.32 (d, 1H), 8.61 (d, 1H), 9.03 (d, 1H). |
| 2-15-12 SM = 1-18-6 | 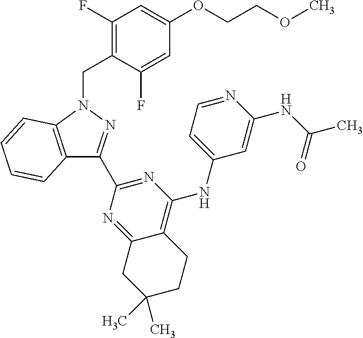 [SMILES: COCCOc1cc(F)c(Cn2nc(-c3nc4c(c(Nc5ccnc(NC(C)=O)c5)n3)CCC(C)(C)C4)c3ccccc32)c(F)c1] | N-{4-[(2-{1-[2,6-difluoro- 4-(2- methoxyethoxy)benzyl]- 1H-indazol-3-yl}-7,7- dimethyl-5,6,7,8- tetrahydroquinazolin-4- yl)amino]pyridin-2- yl}acetamide | ¹H-NMR (400 MHz, DMSO- d₆): δ [ppm] = 1.02 (s, 6H), 1.65 (m, 2H), 2.08 (s, 3H), 2.64 (br. s., 2H), 2.72 (m, 2H), 3.26 (s, 3H), 3.61 (m, 2H), 4.11 (m, 2H), 5.69 (s, 2H), 6.82 (m, 2H), 7.20 (dd, 1H), 7.46 (dd, 1H), 7.80 (d, 1H), 8.13 (d, 1H), 8.22 (dd, 1H), 8.26 (br. s., 1H), 8.46 (d, 1H), 8.90 (br. s., 1H), 10.33 (br. s., 1H). |
| 2-15-13 SM = 1-18-6 | 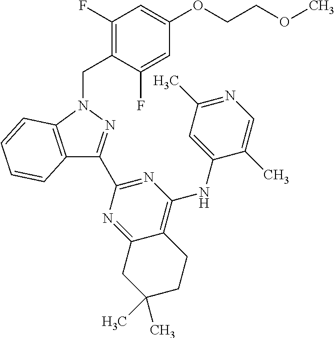 [SMILES: COCCOc1cc(F)c(Cn2nc(-c3nc4c(c(Nc5cc(C)ncc5C)n3)CCC(C)(C)C4)c3ccccc32)c(F)c1] | 2-{1-[2,6-difluoro-4-(2- methoxyethoxy)benzyl]- 1H-indazol-3-yl}-N-(2,5- dimethylpyridin-4-yl)- 7,7-dimethyl-5,6,7,8- tetrahydroquinazolin-4- amine | ¹H-NMR (400 MHz, DMSO- d₆): δ [ppm] = 1.03 (s, 6H), 1.67 (m, 2H), 2.20 (s, 3H), 2.44 (s, 3H), 2.61 (br. s., 2H), 2.70 (m, 2H), 3.27 (s, 3H), 3.61 (m, 2H), 4.11 (m, 2H), 5.67 (s, 2H), 6.79 (m, 2H), 7.10 (dd, 1H), 7.43 (dd, 1H), 7.76 (d, 1H), 7.87 (s, 1H), 8.28 (s, 1H), 8.19 (d, 1H), 8.28 (s, 1H). |

### Example 2-16-1

### Preparation of 2-[1-(4-ethoxy-2,6-difluorobenzyl)-1H-indazol-3-yl]-N-(pyridin-4-yl)-5-{2-[(2,2,2-trifluoroethyl)amino]ethyl}pyrimidin-4-amine

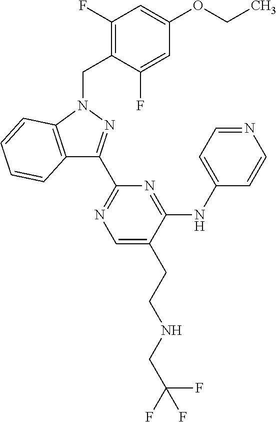 [SMILES: CCOc1cc(F)c(Cn2nc(-c3ncc(CCNCC(F)(F)F)c(Nc4ccncc4)n3)c3ccccc32)c(F)c1]

135 mg of 5-(2-aminoethyl)-2-[1-(4-ethoxy-2,6-difluorobenzyl)-1H-indazol-3-yl]-N-(pyridin-4-yl)pyrimidin-4-amine 2-9-2 (0.27 mmol, 0.86 eq.) were dissolved in 1.2 mL DMF. 121 μL N,N-diisopropylethylamine (0.70 mmol, 2.2 eq.) and 94 mg 2,2,2-trifluoroethyl trifluoromethanesulfonate (0.40 mmol, 1.3 eq.) were added. The reaction mixture was stirred for 20 h at room temperature. The reaction mixture was partitioned between dichloromethane and diluted sodiumhydrogencarbonate solution. The aqueous layer was extracted twice with dichloromethane. The combined organic layers were washed with water and brine and dried over a water-resistant filter followed by evaporation under reduced pressure. The crude product was purified by chromatography to provide the desired compound in 99% purity: 14 mg, 0.02 mmol, 9%.

¹H-NMR (400 MHz, DMSO-d₆): δ [ppm]=1.27 (t, 3H), 2.82-2.96 (m, 4H), 3.26-3.40 (m, 3H), 4.02 (q, 2H), 5.69 (s, 2H), 6.73-6.82 (m, 2H), 7.25 (t, 1H), 7.43-7.52 (m, 1H), 7.84 (d, 1H), 7.97-8.08 (m, 2H), 8.36-8.49 (m, 4H), 9.49 (br. s, 1H).

### Example 2-17-1

### Preparation of 2-[1-(4-ethoxy-2,6-difluorobenzyl)-1H-indazol-3-yl]-5-(piperidin-4-yl)-N-(pyridin-4-yl)pyrimidin-4-amine

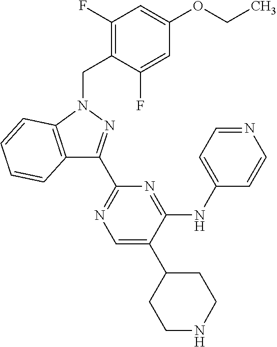 [SMILES: CCOc1cc(F)c(Cn2nc(-c3ncc(C4CCNCC4)c(Nc4ccncc4)n3)c3ccccc32)c(F)c1]

182 mg of 68% pure tert-butyl 4-{2-[1-(4-ethoxy-2,6-difluorobenzyl)-1H-indazol-3-yl]-4-(pyridin-4-ylamino)pyrimidin-5-yl}piperidine-1-carboxylate 2-2-30 (0.14 mmol, 1.0 eq.) were dissolved in 0.88 mL dioxane. 0.21 mL of hydrochloric acid in dioxane (4 M, 0.82 mmol, 4.8 eq.) were added. The reaction mixture was stirred for 72 h room temperature. The reaction mixture was diluted with ethyl acetate and saturated sodiumhydrogencarbonate-solution and stirred for 5 min. The layers were separated and the aqueous layer was extracted twice with ethyl acetate. The combined organic layers were dried in use of a waterresistant filter and the filtrate was evaporated under reduced pressure. The crude product was purified by HPLC (basic) to provide the target compound with 98% purity: 38 mg, 0.07 mmol, 50%.

¹H-NMR (300 MHz, DMSO-d₆): δ [ppm]=1.28 (t, 3H), 1.48-1.65 (m, 2H), 1.75 (br. s., 2H), 2.73 (br. t, 2H), 3.06 (d, 4H), 4.03 (q, 2H), 5.69 (s, 2H), 6.70-6.84 (m, 2H), 7.23 (t, 1H), 7.48 (t, 1H), 7.85 (d, 1H), 7.99-8.09 (m, 2H), 8.36-8.46 (m, 3H), 8.49 (s, 1H), 8.92 (s, 1H)

The following compounds were prepared according to the same procedure from the indicated starting materials (SM=starting material):

| | | | |
|---|---|---|---|
| 2-17-2 SM = 2-2-31 | 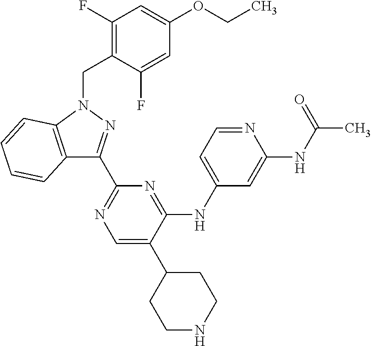 [SMILES: CCOc1cc(F)c(Cn2nc(-c3ncc(C4CCNCC4)c(Nc4ccnc(NC(C)=O)c4)n3)c3ccccc32)c(F)c1] | N-[4-({2-[1-(4-ethoxy- 2,6-difluorobenzyl)-1H- indazol-3-yl]-5- (piperidin-4- yl)pyrimidin-4- yl}amino)pyridin-2- yl]acetamide | ¹H-NMR (400MHz, DMSO-d₆): δ [ppm] = 1.29 (t, 3H), 1.46-1.62 (m, 2H), 1.67-1.84 (m, 2H), 2.09 (s, 3H), 2.64-2.78 (m, 2H), 2.97-3.09 (m, 2H), 3.11-3.26 (m, 1H), 4.04 (q, 2H), 5.69 (s, 2H), 6.69-6.86 (m, 2H), 7.20 (t, 1H), 7.48 (t, 1H), 7.83 (d, 1H), 8.06-8.18 (m, 2H), 8.24 (s, 1H), 8.41 (d, 1H), 8.49 (s, 1H), 9.11 (br. s, 1H), 10.35 (s, 1H). |
| 2-17-3 SM = 2-2-33 | 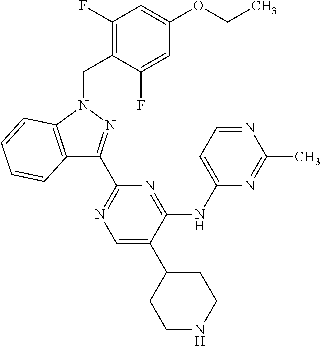 [SMILES: CCOc1cc(F)c(Cn2nc(-c3ncc(C4CCNCC4)c(Nc4ccnc(C)n4)n3)c3ccccc32)c(F)c1] | 2-[1-(4-ethoxy-2,6- difluorobenzyl)-1H- indazol-3-yl]-N-(2- methylpyrimidin-4-yl)-5- (piperidin-4- yl)pyrimidin-4-amine | ¹H-NMR (400MHz, DMSO-d₆): δ [ppm] = 1.15-1.25 (m, 1H), 1.29 (t, 3H), 1.49-1.63 (m, 2H), 1.76 (d, 2H), 2.57 (s, 3H), 2.66-2.73 (m, 2H), 3.03 (d, 2H), 4.04 (q, 2H), 5.71 (s, 2H), 6.76-6.85 (m, 2H), 7.27 (t, 1H), 7.47-7.54 (m, 1H), 7.87 (d, 1H), 8.40-8.50 (m, 3H), 8.59 (s, 1H), 9.79 (br. s, 1H). -NH Signal missing |
| 2-17-4 SM = 2-2-34 | 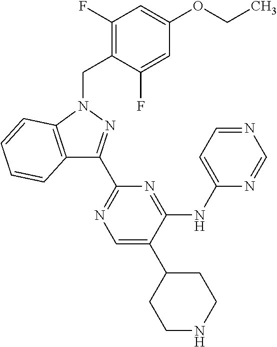 [SMILES: CCOc1cc(F)c(Cn2nc(-c3ncc(C4CCNCC4)c(Nc4ccncn4)n3)c3ccccc32)c(F)c1] | 2-[1-(4-ethoxy-2,6- difluorobenzyl)-1H- indazol-3-yl]-5- (piperidin-4-yl)-N- (pyrimidin-4- yl)pyrimidin-4-amine | ¹H-NMR (400MHz, DMSO-d₆): δ [ppm] = 1.15-1.26 (m, 1H), 1.29 (t, 3H), 1.50-1.62 (m, 2H), 1.72-1.80 (m, 2H), 2.64- 2.76 (m, 2H), 2.99- 3.08 (m, 2H), 4.04 (d, 2H), 5.71 (s, 2H), 6.77- 6.86 (m, 2H), 7.28 (t, 1H), 7.47-7.54 (m, 1H), 7.88 (d, 1H), 8.47 (d, 1H), 8.54-8.59 (m, 1H), 8.59-8.66 (m, 2H), 8.88 (d, 1H), 9.90 (br. s, 1H). -NH Signal is missing. |
| 2-17-5 SM = 2-2-37 | 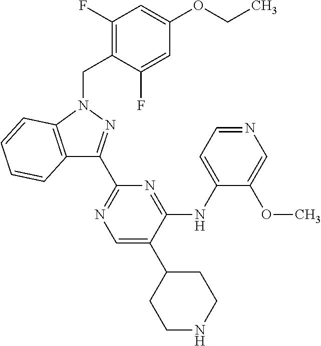 [SMILES: CCOc1cc(F)c(Cn2nc(-c3ncc(C4CCNCC4)c(Nc4ccncc4OC)n3)c3ccccc32)c(F)c1] | 2-[1-(4-ethoxy-2,6- difluorobenzyl)-1H- indazol-3-yl]-N-(3- methoxypyridin-4-yl)-5- (piperidin-4- yl)pyrimidin-4-amine | ¹H-NMR (400MHz, DMSO-d₆): δ [ppm] = 1.29 (t, 3H), 1.57-1.71 (m, 2H), 1.82-1.94 (m, 2H), 2.68-2.78 (m, 2H), 2.80- 2.89 (m, 1H), 3.08- 3.16 (m, 2H), 3.99 (s, 3H), 4.04 (q, 2H), 5.70 (s, 2H), 6.76-6.86 (m, 2H), 7.20-7.27 (m, 1H), 7.45- 7.53 (m, 1H), 7.86 (d, 1H), 8.08 (s, 1H), 8.18- 8.23 (m, 1H), 8.33-8.40 (m, 2H), 8.47 (s, 1H), 8.63 (d, 1H). -NH not detectable. |
| 2-17-6 SM = 2-2-38 | 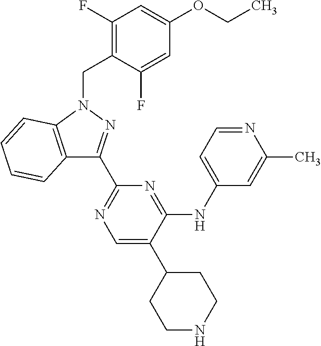 [SMILES: CCOc1cc(F)c(Cn2nc(-c3ncc(C4CCNCC4)c(Nc4ccnc(C)c4)n3)c3ccccc32)c(F)c1] | 2-[1-(4-ethoxy-2,6- difluorobenzyl)-1H- indazol-3-yl]-N-(2- methylpyridin-4-yl)-5- (piperidin-4- yl)pyrimidin-4-amine | ¹H-NMR (400MHz, DMSO-d₆): δ [ppm] = 1.28 (t, 3H), 1.48-1.62 (m, 2H), 1.72-1.80 (m, 2H), 2.43 (s, 3H), 2.67-2.77 (m, 2H), 3.00-3.08 (m, 2H), 3.09-3.19 (m, 1H), 4.03 (q, 2H), 5.69 (s, 2H), 6.71-6.81 (m, 2H), 7.22 (t, 1H), 7.44-7.50 (m, 1H), 7.80-7.88 (m, 3H), 8.29 (d, 1H), 8.41-8.49 (m, 2H), 8.81 (s, 1H). -NH not detectable. |
| 2-17-7 SM = 2-2-38 | 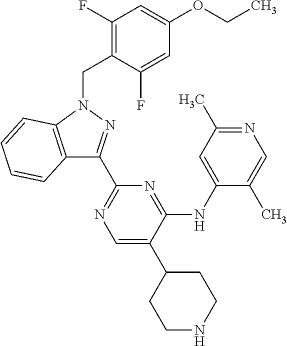 [SMILES: CCOc1cc(F)c(Cn2nc(-c3ncc(C4CCNCC4)c(Nc4cc(C)ncc4C)n3)c3ccccc32)c(F)c1] | N-(2,5-dimethylpyridin- 4-yl)-2-[1-(4-ethoxy-2,6- difluorobenzyl)-1H- indazol-3-yl]-5- (piperidin-4- yl)pyrimidin-4-amine | LC-MS (Method 5): retention time: 1.40 min MS ES+: 571 [M + H]⁺ |
| 2-17-8 SM = 2-2-40 | 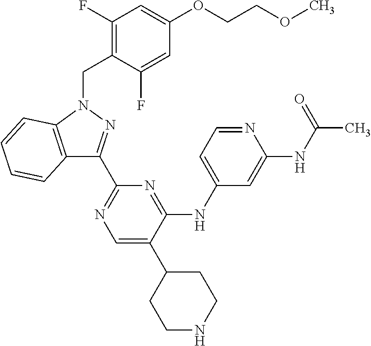 [SMILES: COCCOc1cc(F)c(Cn2nc(-c3ncc(C4CCNCC4)c(Nc4ccnc(NC(C)=O)c4)n3)c3ccccc32)c(F)c1] | N-(4-{[2-{1-[2,6- difluoro-4-(2- methoxyethoxy) benzyl]- 1H-indazol-3-yl}-5- (piperidin-4- yl)pyrimidin-4- yl]amino}pyridin-2- yl)acetamide | ¹H-NMR (400 MHz, DMSO-d₆): δ [ppm] = 1.55 (dddd, 2H), 1.76 (br. d., 2H), 2.09 (s, 3H), 2.71 (m, 2H), 3.03 (m, 2H), 3.20 (m, 1H), 3.26 (s, 3H), 3.61 (m, 2H), 4.11 (m, 2H), 5.69 (s, 2H), 6.81 (m, 2H), 7.19 (dd, 1H), 7.47 (dd, 1H), 7.82 (d, 1H), 8.12 (dd, 1H), 8.14 (d, 1H), 8.24 (d, 1H), 8.40 (d, 1H), 8.48 (s, 1H), 9.13 (br. s., 1H), 10.36 (br. s., 1H). |
| 2-17-9 SM = 2-2-41 | 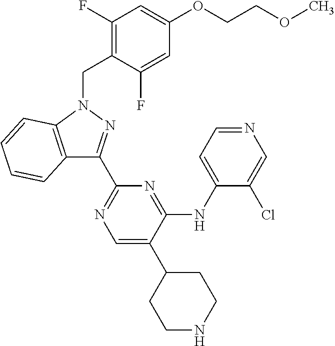 [SMILES: COCCOc1cc(F)c(Cn2nc(-c3ncc(C4CCNCC4)c(Nc4ccncc4Cl)n3)c3ccccc32)c(F)c1] | N-(3-chloropyridin-4-yl)- 2-{1-[2,6-difluoro-4-(2- methoxyethoxy)benzyl]- 1H-indazol-3-yl}-5- (piperidin-4- yl)pyrimidin-4-amine | ¹H-NMR (400 MHz, DMSO-d₆): δ [ppm] = 1.60 (dddd, 2H), 1.87 (br. d., 2H), 2.96 (tt, 1H), 2.68 (m, 2H), 3.08 (m, 2H), 3.26 (s, 3H), 3.61 (m, 2H), 4.11 (m, 2H), 5.67 (s, 2H), 6.81 (m, 2H), 7.11 (dd, 1H), 7.44 (dd, 1H), 7.81 (d, 1H), 8.07 (d, 1H), 8.31 (d, 1H), 8.48 (d, 1H), 8.48 (d, 1H), 8.66 (s, 1H). |
| 2-17-10 SM = 2-2-42 | 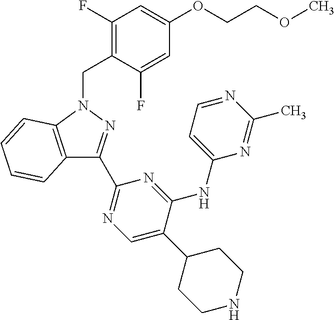 [SMILES: COCCOc1cc(F)c(Cn2nc(-c3ncc(C4CCNCC4)c(Nc4ccnc(C)n4)n3)c3ccccc32)c(F)c1] | 2-{1-[2,6-difluoro-4-(2- methoxyethoxy)benzyl]- 1H-indazol-3-yl}-N-(2- methylpyrimidin-4-yl)-5- (piperidin-4- yl)pyrimidin-4-amine | ¹H-NMR (400 MHz, DMSO-d₆): δ [ppm] = 1.54 (dddd, 2H), 1.74 (br. d., 2H), 2.56 (s, 3H), 2.69 (m, 2H), 3.00 (m, 2H), 3.26 (s, 3H), 3.37 (m, 1H), 3.61 (m, 2H), 4.11 (m, 2H), 5.71 (s, 2H), 6.84 (m, 2H), 7.27 (dd, 1H), 7.50 (dd, 1H), 7.87 (d, 1H), 8.43 (d, 1H), 8.46 (d, 1H), 8.48 (d, 1H), 8.58 (s, 1H). |

### Example 2-18-1

### Preparation of 2-[1-(4-ethoxy-2,6-difluorobenzyl)-1H-indazol-3-yl]-N-(pyridin-4-yl)-5,6,7,8-tetrahydropyrido[3,4-d]pyrimidin-4-amine

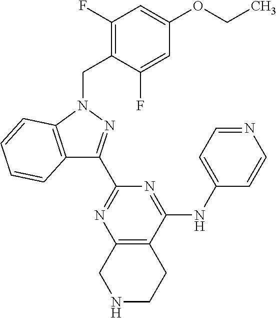 [SMILES: CCOc1cc(F)c(Cn2nc(-c3nc4c(c(Nc5ccncc5)n3)CCNC4)c3ccccc32)c(F)c1]

72 mg 7-Benzyl-2-[1-(4-ethoxy-2,6-difluorobenzyl)-1H-indazol-3-yl]-N-(pyridin-4-yl)-5,6,7,8-tetrahydropyrido[3,4-d]pyrimidin-4-amine 2-12-15 (0.119 mmol, 1.0 eq.) were dissolved in 1.00 mL dichloromethane. Then, 17 μL of 1-chloroethyl carbonochloridate (0.155 mmol, 1.3 eq.) were added under ice-cooling. HPLC-analysis indicated low conversion. The volatile components were removed by the use of a rotoevaporator. Then, 1.00 mL dichloromethane, 83 μL diisopropylethylamine (0.477 mmol, 4.0 eq.) and 39 μL of 1-chloroethyl carbonochloridate (0.358 mmol, 3.0 eq.) were added and the reaction mixture was stirred for 45 min at ambient temperature. Again, HPLC-analysis indicated low conversion and the volatile components were removed by the use of a rotoevaporator. The remaining mixture was dissolved in 1 mL of methanol and 179 μL hydrogen chloride as a solution in dioxane (4M, 0.716 mmol, 6 eq.) were added. The reaction mixture was stirred for 45 min at reflux temperature. The reaction mixture was concentrated and the crude product was purified by HPLC (basic) to provide 5 mg of the target compound (8% of theory) in 90% purity (HPLC area-%).

¹H-NMR (400 MHz, DMSO-d₆): δ [ppm]=1.30 (t, 3H), 3.08 (t, 2H), 3.90 (s, 2H), 4.05 (q, 2H), 5.71 (s, 2H), 6.81 (d, 2H), 7.21-7.32 (m, 1H), 7.49 (t, 1H), 7.85 (d, 1H), 8.09 (d, 2H), 8.19 (s, 1H), 8.41 (d, 2H), 8.48 (d, 1H), 8.79 (s, 1H), 2H not assigned.

### Example 2-19-1

### Preparation of 2-[1-(4-ethoxy-2,6-difluorobenzyl)-1H-indazol-3-yl]-N-(pyridin-4-yl)-5,7-dihydrothieno[3,4-d]pyrimidin-4-amine

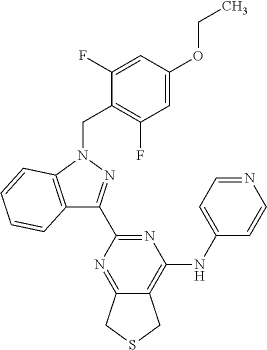 [SMILES: CCOc1cc(F)c(Cn2nc(-c3nc4c(c(Nc5ccncc5)n3)CSC4)c3ccccc32)c(F)c1]

100 mg (0.22 mmol) 4-chloro-2-[1-(4-ethoxy-2,6-difluorobenzyl)-1H-indazol-3-yl]-5,7-dihydrothieno[3,4-d]pyrimidine 1-20-1, 23 mg (0.24 mmol) 4-aminopyridine, 139 mg (0.65 mmol) potassium phosphate, 7 mg (0.03 mmol) palladium acetate and 20 mg (0.03 mmol) (rac)-2,2′-bis(diphenylphosphino)-1,1′-binaphthyl were dissolved under inert atmosphere in 1.68 mL N,N-dimethylformamide. The suspension was heated to 100° C. overnight, cooled to room temperature and extracted with dichloromethane and water. The organic layer was filtered through a silicon filter and concentrated under reduced pressure. The crude product was purified by HPLC to yield 33 mg (28% yield, purity 94%) of 2-[1-(4-ethoxy-2,6-difluorobenzyl)-1H-indazol-3-yl]-N-(pyridin-4-yl)-5,7-dihydrothieno[3,4-d]pyrimidin-4-amine.

¹H-NMR (400 MHz, DMSO-d₆): δ [ppm]=1.30 (t, 3H) 4.05 (q, 2H) 4.26 (s, 2H) 4.35 (s, 2H) 5.72 (s, 2H) 6.80 (s, 1H) 6.82 (s, 1H) 7.14-7.41 (m, 1H) 7.42-7.72 (m, 2H) 7.83-7.98 (m, 1H) 8.01-8.20 (m, 2H) 8.40-8.61 (m, 2H) 9.27 (s, 1H).

The following compounds were prepared according to the same procedure from the indicated starting materials (SM=starting material):

| | | | |
|---|---|---|---|
| 2-19-2 SM = 1-20-1 | 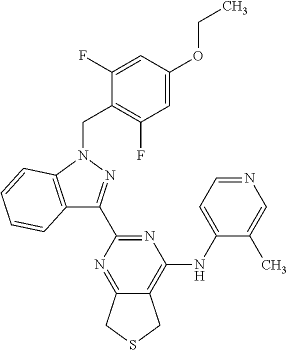 [SMILES: CCOc1cc(F)c(Cn2nc(-c3nc4c(c(Nc5ccncc5C)n3)CSC4)c3ccccc32)c(F)c1] | 2-[1-(4-ethoxy-2,6- difluorobenzyl)-1H- indazol-3-yl]-N-(3- methylpyridin-4-yl)-5,7- dihydrothieno[3,4- d]pyrimidin-4-amine | ¹H-NMR (400MHz, DMSO-d₆): δ [ppm] = 1.28 (t, 3 H) 2.22 (s, 3 H) 4.03 (q, 2 H) 4.25 (s, 2 H) 4.30 (s, 2 H) 5.65 (s, 2 H) 6.74 (s, 1 H) 6.77 (s, 1 H) 6.98- 7.18 (m, 1 H) 7.42 (t, 1 H) 7.71 (d, 1 H) 7.74- 7.83 (m, 1 H) 8.02 (d, 1 H) 8.40 (d, 1 H) 8.43- 8.50 (m, 1 H) 8.64 (s, 1 H). |
| 2-19-3 SM = 1-20-1 | 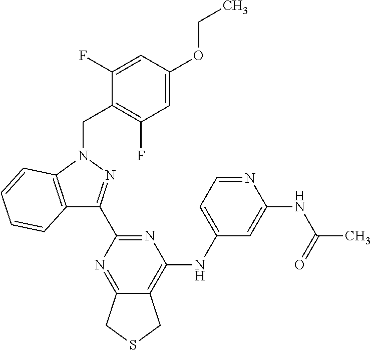 [SMILES: CCOc1cc(F)c(Cn2nc(-c3nc4c(c(Nc5ccnc(NC(C)=O)c5)n3)CSC4)c3ccccc32)c(F)c1] | N-[4-({2-[1-(4-ethoxy- 2,6-difluorobenzyl)-1H- indazol-3-yl]-5,7- dihydrothieno[3,4- d]pyrimidin-4- yl}amino)pyridin-2- yl]acetamide | ¹H-NMR (400MHz, DMSO-d₆): δ [ppm] = 1.28 (t, 3 H) 2.08 (s, 3 H) 4.03 (q, 2 H) 4.28 (s, 2 H) 4.31 (s, 2 H) 5.70 (s, 2 H) 6.64- 6.91 (m, 2 H) 7.23 (t, 1 H) 7.39-7.57 (m, 1 H) 7.83 (d, 1 H) 8.15 (d, 1 H) 8.22 (s, 1 H) 8.30 (dd, 1 H) 8.45 (d, 1 H) 9.40 (s, 1 H) 10.35 (s, 1 H). |
| 2-19-4 SM = 1-20-1 | 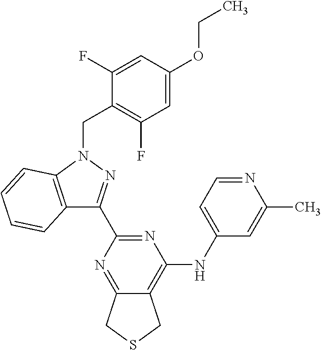 [SMILES: CCOc1cc(F)c(Cn2nc(-c3nc4c(c(Nc5ccnc(C)c5)n3)CSC4)c3ccccc32)c(F)c1] | 2-[1-(4-ethoxy-2,6- difluorobenzyl)-1H- indazol-3-yl]-N-(2- methylpyridin-4-yl)-5,7- dihydrothieno[3,4- d]pyrimidin-4-amine | ¹H-NMR (400MHz, DMSO-d₆): δ [ppm] = 1.29 (t, 3 H) 2.66 (s, 3 H) 4.04 (q, 2 H) 4.35 (br. s., 2 H) 4.42 (br. s., 2 H) 5.77 (s, 2 H) 6.78 (s, 1 H) 6.81 (s, 1 H) 7.26-7.43 (m, 1 H) 7.47-7.66 (m, 1 H) 7.88 (d, 1 H) 8.15 (d, 1 H) 8.41-8.59 (m, 2 H) 8.60- 8.78 (m, 1 H) 10.28 (s, 1 H). |
| 2-19-5 SM = 1-20-2 Addi- tion of dioxane | 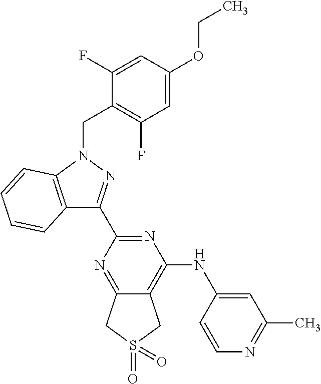 [SMILES: CCOc1cc(F)c(Cn2nc(-c3nc4c(c(Nc5ccnc(C)c5)n3)CS(=O)(=O)C4)c3ccccc32)c(F)c1] | 2-[1-(4-ethoxy-2,6- difluorobenzyl)-1H- indazol-3-yl]-N-(2- methylpyridin-4-yl)-5,7- dihydrothieno[3,4- d]pyrimidin-4-amine 6,6-dioxide | ¹H-NMR (400MHz, DMSO-d₆): δ [ppm] = 1.28 (t, 3 H) 2.46 (s, 3 H) 4.03 (q, 2 H) 4.62 (s, 2 H) 4.72 (s, 2 H) 5.73 (s, 2 H) 6.76 (s, 1 H) 6.79 (s, 1 H) 7.28 (t, 1 H) 7.51 (t, 1 H) 7.72 (d, 1 H) 7.77-8.00 (m, 2 H) 8.34 (d, 1 H) 8.46 (d, 1 H) 9.31 (br. s., 1 H). |
| 2-19-6 SM = 1-13-1 Addi- tion of dioxane | 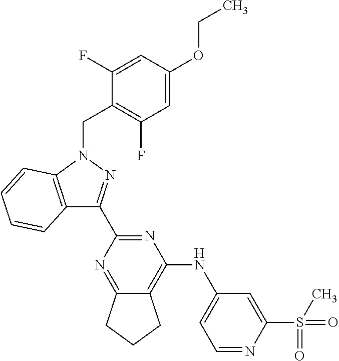 [SMILES: CCOc1cc(F)c(Cn2nc(-c3nc4c(c(Nc5ccnc(S(C)(=O)=O)c5)n3)CCC4)c3ccccc32)c(F)c1] | 2-[1-(4-ethoxy-2,6- difluorobenzyl)-1H- indazol-3-yl]-N-[2- (methylsulfonyl)pyridin- 4-yl]-6,7-dihydro-5H- cyclopenta[d]pyrimidin- 4-amine | ¹H-NMR (400MHz, DMSO-d₆): δ [ppm] = 1.28 (t, 3H) 2.09-2.17 (m, 2 H) 2.92-3.06 (m, 4 H) 3.27 (s, 3 H) 4.03 (q, 2 H) 5.71 (s, 2 H) 6.78 (s, 1 H) 6.81 (s, 1 H) 7.27 (t, 1 H) 7.48 (t, 1 H) 7.82 (d, 1 H) 8.30 (d, 1 H) 8.47 (d, 1 H) 8.56 (d, 1 H) 8.91 (dd, 1 H) 9.70 (s, 1 H). |
| 2-19-7 SM = 1-13-1 | 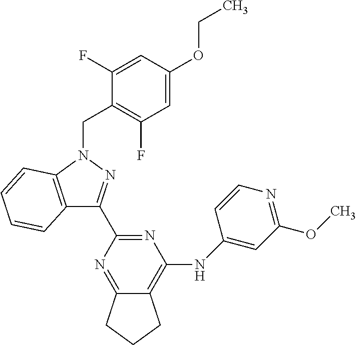 [SMILES: CCOc1cc(F)c(Cn2nc(-c3nc4c(c(Nc5ccnc(OC)c5)n3)CCC4)c3ccccc32)c(F)c1] | 2-[1-(4-ethoxy-2,6- difluorobenzyl)-1H- indazol-3-yl]-N-(2- methoxypyridin-4-yl)- 6,7-dihydro-5H- cyclopenta[d]pyrimidin- 4-amine | ¹H-NMR (300MHz, DMSO-d₆): δ [ppm] = 1.30 (t, 3 H) 2.02-2.21 (m, 2 H) 2.87-3.05 (m, 4 H) 3.87 (s, 3 H) 4.05 (q, 2 H) 5.70 (s, 2 H) 6.77 (s, 1 H) 6.80 (s, 1 H) 7.25 (t, 1 H) 7.42-7.58 (m, 2 H) 7.71 (dd, 1 H) 7.83 (d, 1 H) 8.02 (d, 1 H) 8.51 (d, 1 H) 9.11 (s, 1 H). |
| 2-19-8 SM = 1-13-1 | 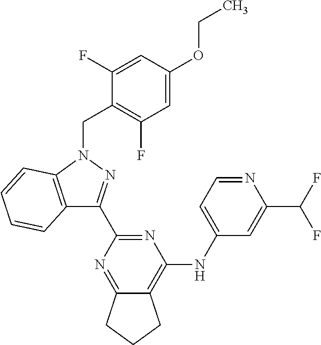 [SMILES: CCOc1cc(F)c(Cn2nc(-c3nc4c(c(Nc5ccnc(C(F)F)c5)n3)CCC4)c3ccccc32)c(F)c1] | N-[2- (difluoromethyl)pyridin- 4-yl]-2-[1-(4-ethoxy-2,6- difluorobenzyl)-1H- indazol-3-yl]-6,7- dihydro-5H- cyclopenta[d]pyrimidin- 4-amine | ¹H-NMR (300MHz, DMSO-d₆): δ [ppm] = 1.27 (m, 3 H) 2.02-2.21 (m, 2 H) 2.94-3.02 (m, 4 H) 4.03 (q, 2 H) 5.70 (s, 2 H) 6.76 (s, 1 H) 6.80 (s, 1 H) 6.87 (t, 1 H) 7.18-7.33 (m, 1 H) 7.48 (t, 1 H) 7.82 (d, 1 H) 8.16 (s, 1 H) 8.46-8.49 (m, 3 H) 9.44 (s, 1 H). |
| 2-19-9 SM = 1-13-8 | 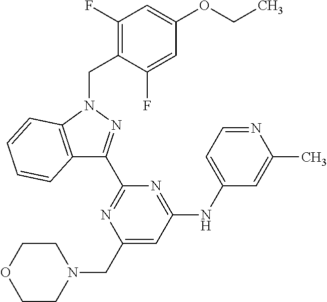 [SMILES: CCOc1cc(F)c(Cn2nc(-c3nc(CN4CCOCC4)cc(Nc4ccnc(C)c4)n3)c3ccccc32)c(F)c1] | 2-[1-(4-ethoxy-2,6- difluorobenzyl)-1H- indazol-3-yl]-N-(2- methylpyridin-4-yl)-6- (morpholin-4- ylmethyl)pyrimidin-4- amine | ¹H-NMR (400MHz, DMSO-d₆): δ [ppm] = 1.28 (t, 3 H) 2.44 (s, 3 H) 2.51- 2.58 (m, 4 H) 3.52- 3.74 (m, 6 H) 4.03 (q, 2 H) 5.71 (s, 2 H) 6.75 (s, 1 H) 6.77 (s, 1 H) 6.96 (s, 1 H) 7.29 (t, 1 H) 7.49 (td, 1 H) 7.60 (dd, 1 H) 7.84 (d, 1 H) 7.88 (d, 1 H) 8.26 (d, 1 H) 8.55 (d, 1 H) 9.96 (s, 1 H). |
| 2-19-10 SM = 1-13-8 | 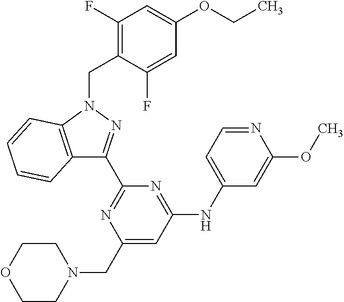 [SMILES: CCOc1cc(F)c(Cn2nc(-c3nc(CN4CCOCC4)cc(Nc4ccnc(OC)c4)n3)c3ccccc32)c(F)c1] | 2-[1-(4-ethoxy-2,6- difluorobenzyl)-1H- indazol-3-yl]-N-(2- methoxypyridin-4-yl)-6- (morpholin-4- ylmethyl)pyrimidin-4- amine | ¹H-NMR (400MHz, DMSO-d₆): δ [ppm] = 1.28 (m, 3 H) 2.50-2.53 (m, 4 H) 3.51-3.73 (m, 6 H) 3.86 (m, 3 H) 4.03 (q, 2 H) 5.70 (s, 2 H) 6.75 (s, 1 H) 6.77 (s, 1 H) 6.96 (s, 1 H) 7.18-7.31 (m, 1 H) 7.37 (d, 1 H) 7.44-7.56 (m, 2 H) 7.78-7.89 (m, 1 H) 7.95-8.04 (m, 1 H) 8.53 (d, 1 H) 10.01 (s, 1 H). |
| 2-19-11 SM = 1-13-8 + 1-22-1 | 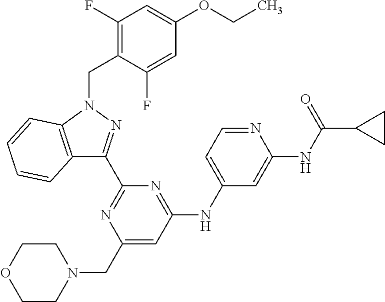 [SMILES: CCOc1cc(F)c(Cn2nc(-c3nc(CN4CCOCC4)cc(Nc4ccnc(NC(=O)C5CC5)c4)n3)c3ccccc32)c(F)c1] | N-[4-({2-[1-(4-ethoxy- 2,6-difluorobenzyl)-1H- indazol-3-yl]-6- (morpholin-4- ylmethyl)pyrimidin-4- yl}amino)pyridin-2- yl]cyclopropane- carboxamide | ¹H-NMR (400MHz, DMSO-d₆): δ [ppm] = 0.78-0.79 (m, 4 H) 1.28 (t, 3 H) 2.02 (quin, 1 H) 2.51- 2.53 (m, 4 H) 3.45-3.68 (m, 6 H) 4.03 (q, 2 H) 5.70 (s, 2 H) 6.76 (s, 1 H) 6.79 (s, 1 H) 6.99 (s, 1 H) 7.27 (t, 1 H) 7.48 (td, 1 H) 7.76-7.87 (m, 1 H) 8.01 (d, 1 H) 8.14 (d, 1 H) 8.30 (dd, 1 H) 8.53 (d, 1 H) 10.11 (s, 1 H) 10.62 (s, 1 H). |
| 2-19-12 SM = 1-13-8 | 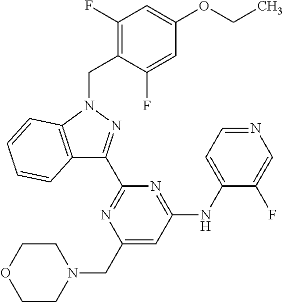 [SMILES: CCOc1cc(F)c(Cn2nc(-c3nc(CN4CCOCC4)cc(Nc4ccncc4F)n3)c3ccccc32)c(F)c1] | 2-[1-(4-ethoxy-2,6- difluorobenzyl)-1H- indazol-3-yl]-N-(3- fluoropyridin-4-yl)-6- (morpholin-4- ylmethyl)pyrimidin-4- amine | ¹H-NMR (400MHz, DMSO-d₆): δ [ppm] = 1.27 (t, 3 H) 2.51-2.57 (m, 4 H) 3.53-3.76 (m, 6 H) 4.02 (q, 2 H) 5.70 (s, 2 H) 6.77 (s, 1 H) 6.80 (s, 1 H) 7.18-7.36 (m, 2 H) 7.49 (ddd, 1 H) 7.85 (d, 1 H) 8.28 (d, 1 H) 8.41- 8.57 (m, 2 H) 8.99 (dd, 1 H) 9.87 (br. s., 1 H). |
| 2-19-13 SM = 1-13-8 | 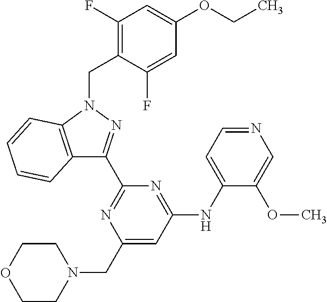 [SMILES: CCOc1cc(F)c(Cn2nc(-c3nc(CN4CCOCC4)cc(Nc4ccncc4OC)n3)c3ccccc32)c(F)c1] | 2-[1-(4-ethoxy-2,6- difluorobenzyl)-1H- indazol-3-yl]-N-(3- methoxypyridin-4-yl)-6- (morpholin-4- ylmethyl)pyrimidin-4- amine | ¹H-NMR (300MHz, DMSO-d₆): δ [ppm] = 1.26 (t, 3 H) 2.49-2.53 (m, 4 H) 3.54-3.77 (m, 6 H) 3.81-4.14 (m, 5 H) 5.69 (s, 2 H) 6.75 (s, 1 H) 6.78 (s, 1 H) 7.18-7.38 (m, 2 H) 7.48 (t, 1 H) 7.83 (d, 1 H) 8.12 (d, 1 H) 8.30 (s, 1 H) 8.52 (d, 1 H) 8.87 (d, 1 H) 9.28 (s, 1 H). |
| 2-19-14 SM = 1-20-2 | 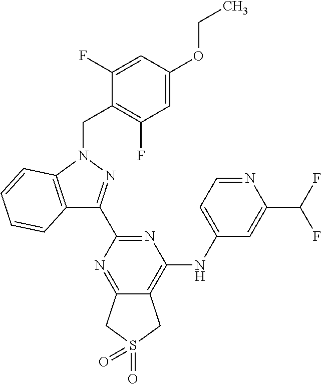 [SMILES: CCOc1cc(F)c(Cn2nc(-c3nc4c(c(Nc5ccnc(C(F)F)c5)n3)CS(=O)(=O)C4)c3ccccc32)c(F)c1] | N-[2- (difluoromethyl)pyridin- 4-yl]-2-[1-(4-ethoxy-2,6- difluorobenzyl)-1H- indazol-3-yl]-5,7- dihydrothieno[3,4- d]pyrimidin-4-amine 6,6-dioxide | ¹H-NMR (300MHz, DMSO-d₆): δ [ppm] = 1.28 (t, 3 H) 4.03 (q, 2 H) 4.65 (s, 2 H) 4.76 (s, 2 H) 5.73 (s, 2 H) 6.77 (s, 1 H) 6.80 (s, 1 H) 6.92 (t, 1 H) 7.27 (t, 1 H) 7.51 (t, 1 H) 7.86 (d, 1 H) 8.09 (d, 1 H) 8.37 (d, 1 H) 8.43 (d, 1 H) 8.57 (d, 1 H) 9.63 (s, 1 H). |
| 2-19-15 SM = 1-30-1 | 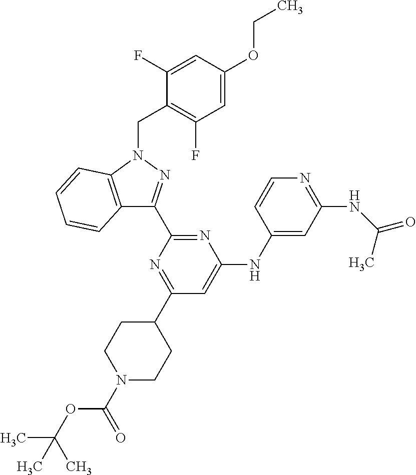 [SMILES: CCOc1cc(F)c(Cn2nc(-c3nc(Nc4ccnc(NC(C)=O)c4)cc(C4CCN(C(=O)OC(C)(C)C)CC4)n3)c3ccccc32)c(F)c1] | tert-butyl 4-(6-{[2- (acetylamino)pyridin-4- yl]amino}-2-[1-(4- ethoxy-2,6- difluorobenzyl)-1H- indazol-3-yl]pyrimidin-4- yl)piperidine-1- carboxylate | ¹H-NMR (300MHz, DMSO-d₆): δ [ppm] = 1.26 (t, 3 H) 1.42 (s, 9 H) 1.54- 1.75 (m, 2 H) 1.92 (d, 2 H) 2.10 (s, 3 H) 2.29- 2.53 (m, 2 H) 2.64 (s, 1 H) 2.80 (m, 1 H) 3.80- 4.20 (m, 2 H) 5.69 (s, 2 H) 6.59-6.85 (m, 3 H) 7.17-7.41 (m, 1 H) 7.41- 7.55 (m, 1 H) 7.67 (dd, 1 H) 7.82 (d, 1 H) 7.99 (m, 1 H) 8.13 (d, 1 H) 8.39-8.63 (m, 2 H) 10.14 (s, 1 H) 10.35 (s, 1 H). |
| 2-19-16 SM = 1-20-5 | 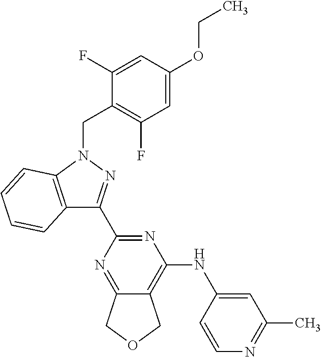 [SMILES: CCOc1cc(F)c(Cn2nc(-c3nc4c(c(Nc5ccnc(C)c5)n3)COC4)c3ccccc32)c(F)c1] | 2-[1-(4-ethoxy-2,6- difluorobenzyl)-1H- indazol-3-yl]-N-(2- methylpyridin-4-yl)-5,7- dihydrofuro[3,4- d]pyrimidin-4-amine | ¹H-NMR (400MHz, DMSO-d₆): δ [ppm] = 1.30 (t, 3 H) 2.24 (s, 3 H) 2.58- 2.77 (m, 4 H) 4.05 (q, 2 H) 5.78 (s, 2 H) 6.77 (s, 1 H) 6.80 (s, 1 H) 7.18 (m, 1 H) 7.33-7.48 (m, 1 H) 7.48-7.70 (m, 1 H) 7.82- 7.93 (m, 1 H) 8.34- 8.53 (m, 2 H) 8.57 (br. s., 1 H). |
| 2-19-17 SM = 1-20-5 | 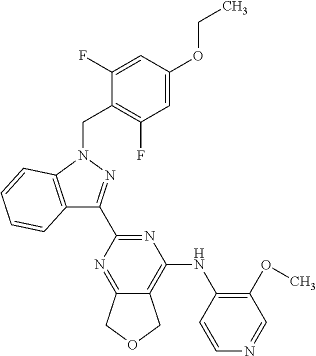 [SMILES: CCOc1cc(F)c(Cn2nc(-c3nc4c(c(Nc5ccncc5OC)n3)COC4)c3ccccc32)c(F)c1] | 2-[1-(4-ethoxy-2,6- difluorobenzyl)-1H- indazol-3-yl]-N-(3- methoxypyridin-4-yl)- 5,7-dihydrofuro[3,4- d]pyrimidin-4-amine | ¹H-NMR (300MHz, DMSO-d₆): δ [ppm] = 1.28 (t, 3H) 3.88-3.97 (m, 3 H) 4.03 (q, 2 H) 4.98 (s, 2 H) 5.12 (s, 2 H) 5.68 (s, 2 H) 6.76 (s, 1 H) 6.79 (s, 1 H) 7.05-7.27 (m, 1 H) 7.47 (t, 1 H) 7.83 (d, 1 H) 8.17 (d, 1 H) 8.23-8.34 (m, 2 H) 8.38 (s, 1 H) 8.71 (s, 1 H). |
| 2-19-18 SM = 1-20-5 | 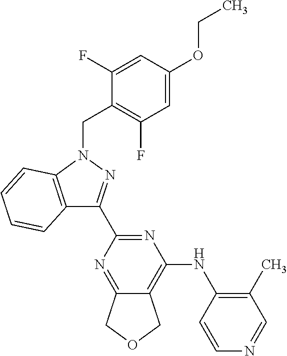 [SMILES: CCOc1cc(F)c(Cn2nc(-c3nc4c(c(Nc5ccncc5C)n3)COC4)c3ccccc32)c(F)c1] | 2-[1-(4-ethoxy-2,6- difluorobenzyl)-1H- indazol-3-yl]-N-(3- methylpyridin-4-yl)-5,7- dihydrofuro[3,4- d]pyrimidin-4-amine | ¹H-NMR (400MHz, DMSO-d₆): δ [ppm] = 1.27 (t, 3 H) 2.44 (s, 3 H) 4.02 (q, 2H) 5.00 (s, 2 H) 5.12 (s, 2 H) 5.71 (s, 2 H) 6.75 (s, 1 H) 6.78 (s, 1 H) 7.27 (t, 1 H) 7.39-7.58 (m, 1 H) 7.73 (dd, 1 H) 7.85 (d, 1 H) 7.92 (d, 1 H) 8.30 (d, 1 H) 8.48 (d, 1 H) 9.47 (s, 1 H). |
| 2-19-19 SM = 1-20-6 | 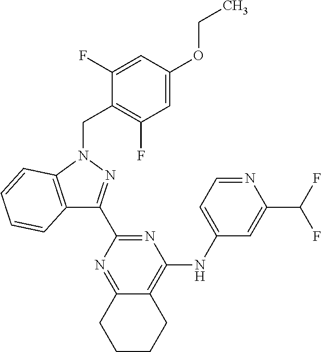 [SMILES: CCOc1cc(F)c(Cn2nc(-c3nc4c(c(Nc5ccnc(C(F)F)c5)n3)CCCC4)c3ccccc32)c(F)c1] | N-[2- (difluoromethyl)pyridin- 4-yl]-2-[1-(4-ethoxy-2,6- difluorobenzyl)-1H- indazol-3-yl]-5,6,7,8- tetrahydroquinazolin-4- amine | ¹H-NMR (300MHz, DMSO-d₆): δ [ppm] = 1.27 (t, 3 H) 1.76-1.95 (m, 4 H) 2.61-2.76 (m, 2 H) 2.78-2.98 (m, 2 H) 4.02 (q, 2 H) 5.70 (s, 2 H) 6.76 (s, 1 H) 6.79 (s, 1 H) 6.87 (t, 1 H) 7.23 (t, 1 H) 7.47 (t, 1 H) 7.80 (d, 1 H) 8.19 (s, 1 H) 8.40-8.56 (m, 3 H) 8.97 (s, 1 H). |
| 2-19-20 SM = 1-20-6 | 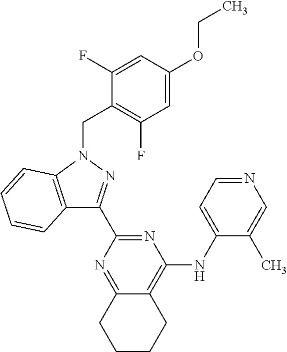 [SMILES: CCOc1cc(F)c(Cn2nc(-c3nc4c(c(Nc5ccncc5C)n3)CCCC4)c3ccccc32)c(F)c1] | 2-[1-(4-ethoxy-2,6- difluorobenzyl)-1H- indazol-3-yl]-N-(3- methylpyridin-4-yl)- 5,6,7,8- tetrahydroquinazolin-4- amine | ¹H-NMR (400MHz, DMSO-d₆): δ [ppm] = 1.28 (t, 3 H) 1.76-1.88 (m, 4 H) 2.21 (s, 3 H) 2.62- 2.72 (m, 2 H) 2.73-2.87 (m, 2 H) 4.03 (q, 2 H) 5.64 (s, 2 H) 6.68-6.83 (m, 2 H) 6.95-7.17 (m, 1 H) 7.40 (ddd, 1 H) 7.74 (d, 1 H) 7.88 (d, 1 H) 7.98 (s, 1 H) 8.09 (d, 1 H) 8.36 (d, 1 H) 8.41 (s, 1 H). |
| 2-19-21 SM = 1-20-6 | 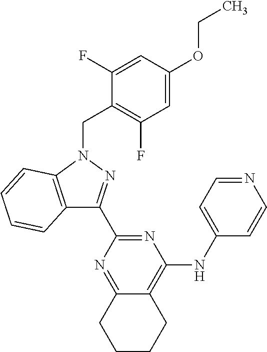 [SMILES: CCOc1cc(F)c(Cn2nc(-c3nc4c(c(Nc5ccncc5)n3)CCCC4)c3ccccc32)c(F)c1] | 2-[1-(4-ethoxy-2,6- difluorobenzyl)-1H- indazol-3-yl]-N-(pyridin- 4-yl)-5,6,7,8- tetrahydroquinazolin-4- amine | ¹H-NMR (400MHz, DMSO-d₆): δ [ppm] = 1.28 (t, 3 H) 1.75-1.92 (m,4 H) 2.61-2.75 (m, 2 H) 2.77-2.91 (m, 2 H) 4.03 (q, 2 H) 5.68 (s, 2 H) 6.77 (s, 1 H) 6.79 (s, 1 H) 7.24 (t, 1 H) 7.43-7.52 (m, 1 H) 7.78-7.88 (m, 1 H) 8.07 (d, 2 H) 8.39 (d, 2 H) 8.48 (d, 1 H) 8.68 (s, 1 H). |

### Example 2-20-1

### Preparation of N-[4-({2-[1-(4-ethoxy-2,6-difluorobenzyl)-1H-indazol-3-yl]-5-[1-(2,2,2-trifluoroethyl)piperidin-4-yl]pyrimidin-4-yl}amino)pyridin-2-yl]acetamide

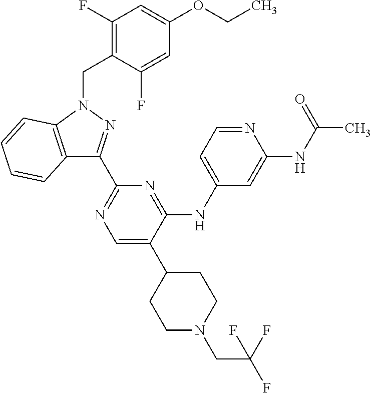 [SMILES: CCOc1cc(F)c(Cn2nc(-c3ncc(C4CCN(CC(F)(F)F)CC4)c(Nc4ccnc(NC(C)=O)c4)n3)c3ccccc32)c(F)c1]

56 mg of N-[4-({2-[1-(4-ethoxy-2,6-difluorobenzyl)-1H-indazol-3-yl]-5-(piperidin-4-yl)pyrimidin-4-yl}amino)pyridin-2-yl]acetamide 2-17-2 (64% pure, 0.06 mmol, 0.86 eq.) was dissolved in 0.3 mL DMF. 27 μL N,N-Diisopropylethylamin (0.16 mmol, 2.2 eq.) and 13 μL 2,2,2-trifluoroethyl trifluoromethanesulfonate (0.09 mmol, 1.3 eq.) were added and the mixture was stirred over night at room temperature. The reaction mixture was diluted with dichloromethane and half-saturated sodium hydrogencarbonate-solution. The layers were separated and the aqueous layer was extracted with dichloromethane twice. The combined organic layers were dried using a waterresistant filter and the filtrate was dried under reduced pressure. The crude product was purified by HPLC to provide the 99 pure target compound: 21 mg, 0.03 mmol, 51%.

¹H-NMR (300 MHz, DMSO-d₆): δ [ppm]=1.28 (t, 3H), 1.63-1.86 (m, 4H), 2.08 (s, 3H), 2.55-2.67 (m, 2H), 2.97-3.28 (m, 5H), 4.02 (q, 2H), 5.67 (s, 2H), 6.70-6.82 (m, 2H), 7.19 (t, 1H), 7.46 (t, 1H), 7.81 (d, 1H), 8.05-8.16 (m, 2H), 8.22 (s, 1H), 8.39 (d, 1H), 8.52 (s, 1H), 9.11 (s, 1H), 10.35 (s, 1H).

The following compounds were prepared according to the same procedure from the indicated starting materials (SM=starting material):

| | | | |
|---|---|---|---|
| 2-20-2 SM = 2-17-3 1.0 eq. 2,2,2- trifluoro- ethyl trifluoro- methane- sulfonate | 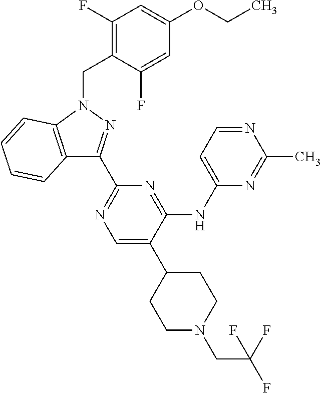 [SMILES: CCOc1cc(F)c(Cn2nc(-c3ncc(C4CCN(CC(F)(F)F)CC4)c(Nc4ccnc(C)n4)n3)c3ccccc32)c(F)c1] | 2-[1-(4-ethoxy-2,6- difluorobenzyl)-1H- indazol-3-yl]-N-(2- methylpyrimidin-4- yl)-5-[1-(2,2,2- trifluoroethyl) piperidin- 4-yl]pyrimidin-4- amine | ¹H-NMR (400MHz, DMSO-d₆): δ [ppm] = 1.29 (t, 3H), 1.67-1.84 (m, 4H), 2.56-2.65 (m, 5H), 3.02 (d, 2H), 3.16-3.30 (m, 3H), 4.04 (q, 2H), 5.71 (s, 2H), 6.74-6.85 (m, 2H), 7.29 (t, 1H), 7.47-7.55 (m, 1H), 7.87 (d, 1H), 8.41-8.50 (m, 3H), 8.65 (s, 1H), 9.77 (br. s, 1H) |
| 2-20-3 SM = 2-17-4 1.0 eq. 2,2,2- trifluoro- ethyl trifluoro- methane- sulfonate | 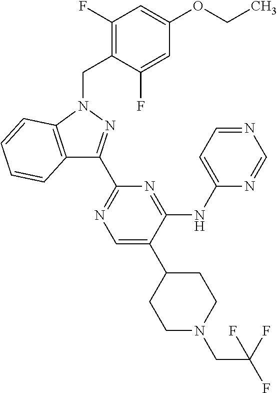 [SMILES: CCOc1cc(F)c(Cn2nc(-c3ncc(C4CCN(CC(F)(F)F)CC4)c(Nc4ccncn4)n3)c3ccccc32)c(F)c1] | 2-[1-(4-ethoxy-2,6- difluorobenzyl)-1H- indazol-3-yl]-N- (pyrimidin-4-yl)-5-[1- (2,2,2- trifluoroethyl) piperidin- 4-yl]pyrimidin-4- amine | ¹H-NMR (400MHz, DMSO-d₆): δ [ppm] = 1.29 (t, 3H), 1.68-1.85 (m, 4H), 2.55-2.65 (m, 2H), 3.03 (d, 2H), 3.15-3.29 (m, 3H), 4.04 (q, 2H), 5.71 (s, 2H), 6.75-6.86 (m, 2H), 7.25-7.32 (m, 1H), 7.48-7.55 (m, 1H), 7.88 (d, 1H), 8.47 (d, 1H), 8.54-8.59 (m, 1H), 8.63 (dd, 1H), 8.67 (s, 1H), 8.88 (d, 1H), 9.88 (s, 1H). |
| 2-20-4 SM = 2-17-5 1.0 eq. 2,2,2- trifluoro- ethyl trifluoro- methane- sulfonate | 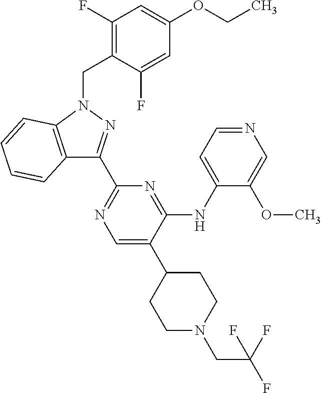 [SMILES: CCOc1cc(F)c(Cn2nc(-c3ncc(C4CCN(CC(F)(F)F)CC4)c(Nc4ccncc4OC)n3)c3ccccc32)c(F)c1] | 2-[1-(4-ethoxy-2,6- difluorobenzyl)-1H- indazol-3-yl]-N-(3- methoxypyridin-4- yl)-5-[1-(2,2,2- trifluoroethyl) piperidin- 4-yl]pyrimidin-4- amine | ¹H-NMR (400MHz, DMSO-d₆): δ [ppm] = 1.28 (t, 3H), 1.72-1.92 (m, 4H), 2.53-2.62 (m, 2H), 2.71-2.82 (m, 1H), 3.05- 3.13 (m, 2H), 3.24 (q, 2H), 3.96 (s, 3H), 4.03 (q, 2H), 5.68 (s, 2H), 6.74- 6.82 (m, 2H), 7.20 (t, 1H), 7.43-7.51 (m, 1H), 7.84 (d, 1H), 8.07 (s, 1H), 8.19 (d, 1H), 8.32 (d, 1H), 8.37 (s, 1H), 8.49 (s, 1H), 8.57 (d, 1H). |
| 2-20-5 SM = 2-17-6 1.5 eq. 2,2,2- trifluoro- ethyl trifluoro- methane- sulfonate | 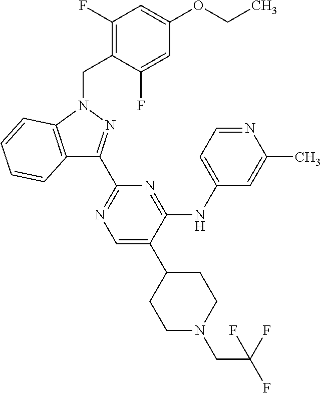 [SMILES: CCOc1cc(F)c(Cn2nc(-c3ncc(C4CCN(CC(F)(F)F)CC4)c(Nc4ccnc(C)c4)n3)c3ccccc32)c(F)c1] | 2-[1-(4-ethoxy-2,6- difluorobenzyl)-1H- indazol-3-yl]-N-(2- methylpyridin-4-yl)- 5-[1-(2,2,2- trifluoroethyl) piperidin- 4-yl]pyrimidin-4- amine | ¹H-NMR (400MHz, DMSO-d₆): δ [ppm] = 1.29 (t, 3H), 1.67-1.87 (m, 4H), 2.45 (s, 3H), 2.58- 2.67 (m, 2H), 3.00-3.12 (m, 3H), 3.21-3.31 (m, 2H), 4.04 (q, 2H), 5.71 (s, 2H), 6.73-6.82 (m, 2H), 7.19-7.28 (m, 1H), 7.45- 7.54 (m, 1H), 7.80- 7.89 (m, 3H), 8.28-8.33 (m, 1H), 8.45 (d, 1H), 8.54 (s, 1H), 8.85 (s, 1H). |
| 2-20-6 SM = 2-17-7 | 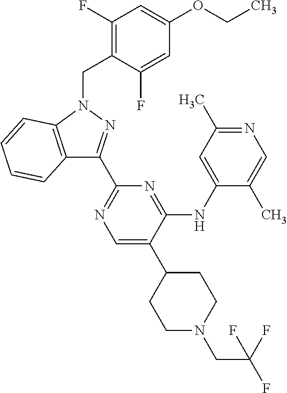 [SMILES: CCOc1cc(F)c(Cn2nc(-c3ncc(C4CCN(CC(F)(F)F)CC4)c(Nc4cc(C)ncc4C)n3)c3ccccc32)c(F)c1] | N-(2,5- dimethylpyridin-4-yl)- 2-[1-(4-ethoxy-2,6- difluorobenzyl)-1H- indazol-3-yl]-5-[1- (2,2,2- trifluoroethyl) piperidin- 4-yl]pyrimidin-4- amine | ¹H-NMR (400MHz, DMSO-d₆): δ [ppm] = 1.29 (t, 3H), 1.67-1.82 (m, 2H), 1.84-1.94 (m, 2H), 2.12 (s, 3H), 2.45 (s, 3H), 2.53-2.63 (m, 2H), 2.86- 2.99 (m, 1H), 3.00- 3.12 (m, 2H), 3.25 (q, 2H), 4.03 (q, 2H), 5.63 (s, 2H), 6.75 (d, 2H), 6.95- 7.08 (m, 1H), 7.35-7.46 (m, 1H), 7.49 (s, 1H), 7.76 (d, 1H), 7.92 (d, 1H), 8.33 (s, 1H), 8.40 (s, 1H), 8.47 (s, 1H). |
| 2-20-7 SM = 2-17-8 | 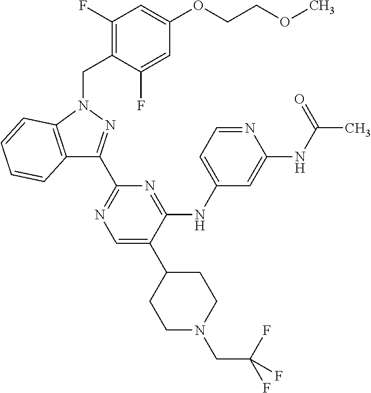 [SMILES: COCCOc1cc(F)c(Cn2nc(-c3ncc(C4CCN(CC(F)(F)F)CC4)c(Nc4ccnc(NC(C)=O)c4)n3)c3ccccc32)c(F)c1] | N-{4-[(2-{1-[2,6- difluoro-4-(2- methoxyethoxy) benzyl]- 1H-indazol-3-yl}- 5-[1-(2,2,2- trifluoroethyl) piperidin- 4-yl]pyrimidin-4- yl)amino]pyridin-2- yl}acetamide | ¹H-NMR (400 MHz, DMSO- d₆): δ [ppm] = 1.72 (m, 2H), 1.81 (br. d., 2H), 2.09 (s, 3H), 2.61 (m, 2H), 3.04 (m, 2H), 3.12 (m, 1H), 3.25 (q, 2H), 3.26 (s, 3H), 3.61 (m, 2H), 4.11 (m, 2H), 5.68 (s, 2H), 6.81 (m, 2H), 7.19 (dd, 1H), 7.47 (dd, 1H), 7.82 (d, 1H), 8.12 (dd, 1H), 8.15 (d, 1H), 8.23 (br. s., 1H), 8.40 (d, 1H), 8.53 (s, 1H), 9.13 (br. s., 1H), 10.37 (br. s., 1H). |
| 2-20-8 SM = 2-17-9 | 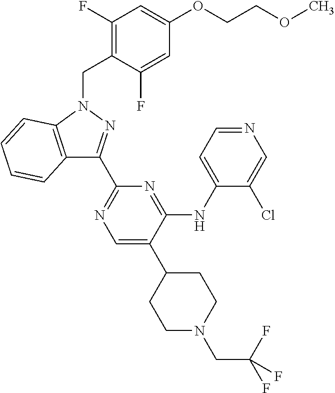 [SMILES: COCCOc1cc(F)c(Cn2nc(-c3ncc(C4CCN(CC(F)(F)F)CC4)c(Nc4ccncc4Cl)n3)c3ccccc32)c(F)c1] | N-(3-chloropyridin-4- yl)-2-{1-[2,6-difluoro- 4-(2- methoxyethoxy) benzyl]- 1H-indazol-3-yl}- 5-[1-(2,2,2- trifluoroethyl) piperidin- 4-yl]pyrimidin-4- amine | ¹H-NMR (400 MHz, DMSO- d₆): δ [ppm] = 1.78 (m, 2H), 1.92 (br. d., 2H), 2.57 (m, 2H), 2.89 (m, 1H), 3.08 (m, 2H), 3.25 (q, 2H), 3.26 (s, 3H), 3.61 (m, 2H), 4.11 (m, 2H), 5.67 (s, 2H), 6.81 (m, 2H), 7.10 (dd, 1H), 7.44 (dd, 1H), 7.80 (d, 1H), 8.03 (d, 1H), 8.26 (d, 1H), 8.50 (d, 1H), 8.53 (s, 1H), 8.58 (s, 1H), 8.67 (br. s., 1H). |
| 2-20-9 SM = 2-17-9 1.2 eq. 3-bromo- 1,1,1- trifluoro- propane, 3 d at 70° C. | 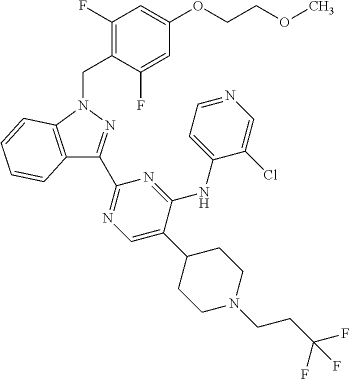 [SMILES: COCCOc1cc(F)c(Cn2nc(-c3ncc(C4CCN(CCC(F)(F)F)CC4)c(Nc4ccncc4Cl)n3)c3ccccc32)c(F)c1] | N-(3-chloropyridin-4- yl)-2-{1-[2,6-difluoro- 4-(2- methoxyethoxy) benzyl]- 1H-indazol-3-yl}- 5-[1-(3,3,3- trifluoropropyl) piperidin- 4-yl]pyrimidin-4- amine | ¹H-NMR (400 MHz, DMSO- d₆): δ [ppm] = 1.74 (m, 2H), 1.94 (br. d., 2H), 2.15 (m, 2H), 2.57 (m, 2H), 2.59 (m, 2H), 2.88 (m, 1H), 3.06 (m, 2H), 3.26 (s, 3H), 3.61 (m, 2H), 4.11 (m, 2H), 5.67 (s, 2H), 6.81 (m, 2H), 7.10 (dd, 1H), 7.44 (dd, 1H), 7.81 (d, 1H), 8.05 (d, 1H), 8.30 (d, 1H), 8.49 (d, 1H), 8.52 (s, 1H), 8.55 (br. s., 1H), 8.67 (s, 1H). |
| 2-20-10 SM = 2-17-10 | 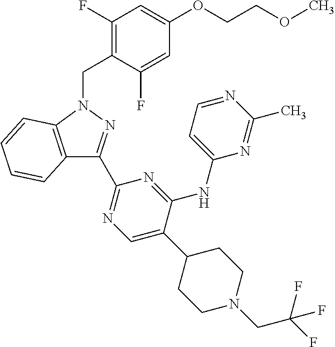 [SMILES: COCCOc1cc(F)c(Cn2nc(-c3ncc(C4CCN(CC(F)(F)F)CC4)c(Nc4ccnc(C)n4)n3)c3ccccc32)c(F)c1] | 2-{1-[2,6-difluoro-4- (2- methoxyethoxy) benzyl]- 1H-indazol-3-yl}- N-(2- methylpyrimidin-4- yl)-5-[1-(2,2,2- trifluoroethyl) piperidin- 4-yl]pyrimidin-4- amine | ¹H-NMR (400 MHz, DMSO- d₆): δ [ppm] = 1.72 (dddd, 2H), 1.78 (br. d., 2H), 2.56 (s, 3H), 2.60 (m, 2H), 3.01 (m, 2H), 3.24 (q, 2H), 3.25 (m, 1H), 3.26 (s, 3H), 3.61 (m, 2H), 4.11 (m, 2H), 5.71 (s, 2H), 6.84 (m, 2H), 7.27 (dd, 1H), 7.50 (dd, 1H), 7.87 (d, 1H), 8.44 (d, 1H), 8.45 (d, 1H), 8.47 (d, 1H), 8.64 (s, 1H), 9.78 (br. s., 1H). |
| 2-20-11 SM = 2-17-10 1.3 eq. 2,2- difluoro- ethyl trifluoro- methane- sulfon | 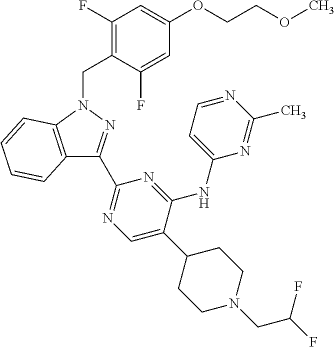 [SMILES: COCCOc1cc(F)c(Cn2nc(-c3ncc(C4CCN(CC(F)F)CC4)c(Nc4ccnc(C)n4)n3)c3ccccc32)c(F)c1] | 5-[1-(2,2- difluoroethyl) piperidin- 4-yl]-2-{1-[2,6- difluoro-4-(2- methoxyethoxy) benzyl]- 1H-indazol-3-yl}- N-(2- methylpyrimidin-4- yl)pyrimidin-4-amine | ¹H-NMR (400 MHz, DMSO- d₆): δ [ppm] = 1.71 (m, 2H), 1.79 (m, 2H), 2.56 (s, 3H), 2.39 (m, 2H), 2.77 (td, 2H), 3.00 (br. d., 2H), 3.19 (m, 1H), 3.26 (s, 3H), 3.61 (m, 2H), 4.11 (m, 2H), 5.71 (s, 2H), 6.18 (tt, 1H), 6.84 (m, 2H), 7.27 (dd, 1H), 7.50 (dd, 1H), 7.87 (d, 1H), 8.43 (d, 1H), 8.45 (d, 1H), 8.47 (d, 1H), 8.63 (s, 1H), 9.77 (br. s., 1H). |
| 2-20-12 SM = 2-17-10 1.2 eq. (bromo- methyl) cyclo propane | 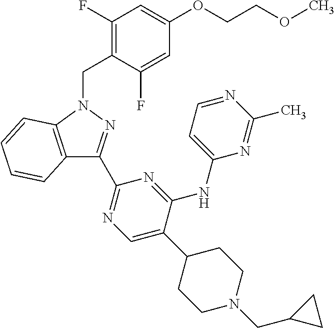 [SMILES: COCCOc1cc(F)c(Cn2nc(-c3ncc(C4CCN(CC5CC5)CC4)c(Nc4ccnc(C)n4)n3)c3ccccc32)c(F)c1] | 5-[1- (cyclopropylmethyl) piperidin-4-yl]-2-{1- [2,6-difluoro-4-(2- methoxyethoxy) benzyl]- 1H-indazol-3-yl}- N-(2- methylpyrimidin-4- yl)pyrimidin-4-amine | ¹H-NMR (400 MHz, DMSO- d₆): δ [ppm] = 0.09 (m, 2H), 0.47 (m, 2H), 0.87 (m, 1 H), 1.71 (dddd, 2H), 1.80 (br. d., 2H), 2.14 (m, 2H), 2.22 (d, 2H), 2.56 (s, 3H), 3.06 (br. d., 2H), 3.17 (m, 1H), 3.26 (s, 3H), 3.61 (m, 2H), 4.11 (m, 2H), 5.70 (s, 2H), 6.83 (m, 2H), 7.27 (dd, 1H), 7.50 (dd, 1H), 7.87 (d, 1H), 8.42 (d, 1H), 8.46 (d, 1H), 8.46 (d, 1H), 8.64 (s, 1H), 9.73 (br. s., 1H). |
| 2-20-13 SM = 2-9-22 1.5 eq. 2,2,2- trifluoro- ethyl trifluoro- methane- sulfonate 1 d rt, 2 h 70° C., 3 d rt, 5 h 100° C., 1.0 eq. 2,2,2- trifluoro- ethyl trifluoro- methane- sulfonate, | 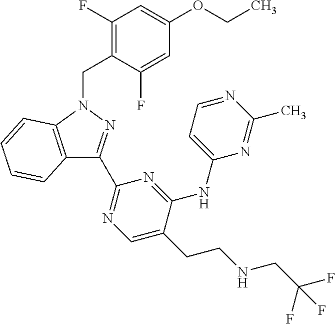 [SMILES: CCOc1cc(F)c(Cn2nc(-c3ncc(CCNCC(F)(F)F)c(Nc4ccnc(C)n4)n3)c3ccccc32)c(F)c1] | 2-[1-(4-ethoxy-2,6- difluorobenzyl)-1H- indazol-3-yl]-N-(2- methylpyrimidin-4- yl)-5-{2-[(2,2,2- trifluoroethyl)amino] ethyl}pyrimidin-4- amine | ¹H-NMR (400 MHz, DMSO- d₆): δ [ppm] = 1.29 (t, 3H), 2.55 (s, 3H), 2.88 (m, 4H), 3.33 (m, 2H), 4.04 (q, 2H), 5.71 (s, 2H), 6.81 (m, 2H), 7.28 (dd, 1H), 7.51 (dd, 1H), 7.88 (d, 1H), 8.47 (d, 1H), 8.47 (d, 1H), 8.51 (s, 1H), 8.56 (d, 1H), 10.31 (br. s., 1H). |
| 2 h 100° C. | | | |
| 2-20-14 SM = 2-9-21 1.5 eq. 2,2,2- trifluoro- ethyl trifluoro- methane- sulfonate | 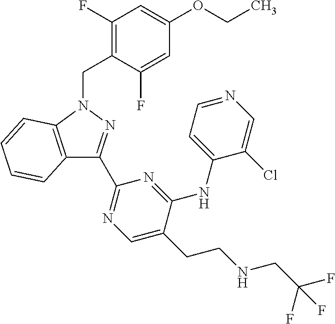 [SMILES: CCOc1cc(F)c(Cn2nc(-c3ncc(CCNCC(F)(F)F)c(Nc4ccncc4Cl)n3)c3ccccc32)c(F)c1] | N-(3-chloropyridin-4- yl)-2-[1-(4-ethoxy- 2,6-difluorobenzyl)- 1H-indazol-3-yl]-5- {2-[(2,2,2- trifluoroethyl)amino] ethyl}pyrimidin-4- amine | ¹H-NMR (400 MHz, DMSO- d₆): δ [ppm] = 1.29 (t, 3H), 2.84 (m, 2H), 2.99 (m, 2H), 3.32 (br. q, 2H), 4.04 (q, 2H), 5.69 (s, 2H), 6.79 (m, 2H), 7.18 (dd, 1H), 7.47 (dd, 1H), 7.84 (d, 1H), 8.24 (d, 1H), 8.42 (d, 1H), 8.50 (s, 1H), 8.53 (d, 1H), 8.62 (s, 1H), 9.49 (br. s., 1H). |
| 2-20-15 SM = 2-9-13 1.5 eq. 2,2,2- trifluoro- ethyl trifluoro- methane- sulfonate | 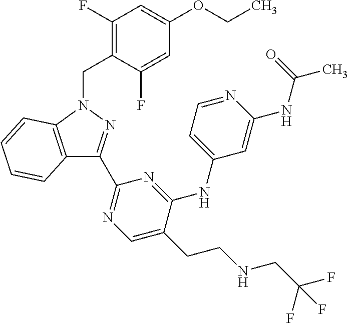 [SMILES: CCOc1cc(F)c(Cn2nc(-c3ncc(CCNCC(F)(F)F)c(Nc4ccnc(NC(C)=O)c4)n3)c3ccccc32)c(F)c1] | N-{4-[(2-[1-(4- ethoxy-2,6- difluorobenzyl)-1H- indazol-3-yl]-5-{2- [(2,2,2- trifluoroethyl)amino] ethyl}pyrimidin-4- yl)amino]pyridin-2- yl}acetamide | ¹H-NMR (400 MHz, DMSO- d₆): δ [ppm] = 1.29 (t, 3H), 2.08 (s, 3H), 2.89 (m, 4H), 3.35 (br. q, 2H), 4.03 (q, 2H), 5.69 (s, 2H), 6.78 (m, 2H), 7.21 (dd, 1H), 7.48 (dd, 1H), 7.84 (d, 1H), 8.15 (d, 1H), 8.20 (d, 1H), 8.24 (dd, 1H), 8.42 (s, 1H), 8.44 (d, 1H), 9.59 (br. s., 1H), 10.36 (br. s., 1H). |
| 2-20-16 SM = 2-9-25 1.5 eq. 2,2,2- trifluoro- ethyl trifluoro- methane- sulfonate, 3 d rt | 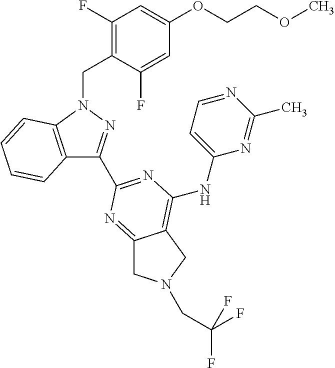 [SMILES: COCCOc1cc(F)c(Cn2nc(-c3nc4c(c(Nc5ccnc(C)n5)n3)CN(CC(F)(F)F)C4)c3ccccc32)c(F)c1] | 2-{1-[2,6-difluoro-4- (2- methoxyethoxy) benzyl]- 1H-indazol-3-yl}- N-(2- methylpyrimidin-4- yl)-6-(2,2,2- trifluoroethyl)-6,7- dihydro-5H- pyrrolo[3,4- d]pyrimidin-4-amine | ¹H-NMR (400 MHz, DMSO- d₆): δ [ppm] = 2.57 (s, 3H), 3.28 (s, 3H), 3.63 (q, 2H), 3.63 (m, 2H), 4.14 (m, 2H), 4.23 (br. s., 2H), 4.29 (br. s., 2H), 5.74 (s, 2H), 6.86 (m, 2H), 7.31 (dd, 1H), 7.53 (dd, 1H), 7.88 (d, 1H), 8.34 (d, 1H), 8.49 (d, 1H), 8.52 (d, 1H), 10.35 (br. s., 1 H). |
| 2-20-17 SM = 2-9-26 1.5 eq. 2,2,2- trifluoro- ethyl trifluoro- methane- sulfonate, 3 d rt | 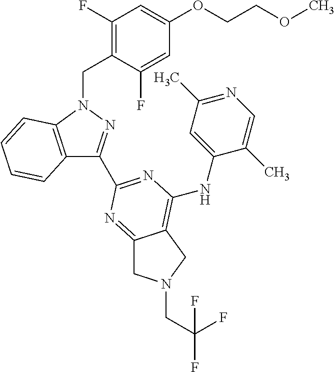 [SMILES: COCCOc1cc(F)c(Cn2nc(-c3nc4c(c(Nc5cc(C)ncc5C)n3)CN(CC(F)(F)F)C4)c3ccccc32)c(F)c1] | 2-{1-[2,6-difluoro-4- (2- methoxyethoxy) benzyl]- 1H-indazol-3-yl}- N-(2,5- dimethylpyridin-4-yl)- 6-(2,2,2- trifluoroethyl)-6,7- dihydro-5H- pyrrolo[3,4- d]pyrimidin-4-amine | ¹H-NMR (400 MHz, DMSO- d₆): δ [ppm] = 2.19 (s, 3H), 2.44 (s, 3H), 3.27 (s, 3H), 3.62 (m, 2H), 3.65 (q, 2H), 4.11 (m, 2H), 4.17 (br. s., 4H), 5.67 (s, 2H), 6.80 (m, 2H), 7.09 (dd, 1H), 7.43 (dd, 1H), 7.53 (s, 1H), 7.77 (d, 1H), 8.15 (d, 1H), 8.31 (s, 1H), 8.73 (br. s., 1H). |
| 2-20-18 SM = 2-9-27 1.5 eq. 2,2,2- trifluoro- ethyl trifluoro- methane- sulfonate, 16 h rt | 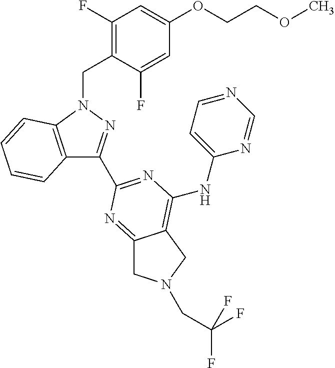 [SMILES: COCCOc1cc(F)c(Cn2nc(-c3nc4c(c(Nc5ccncn5)n3)CN(CC(F)(F)F)C4)c3ccccc32)c(F)c1] | 2-{1-[2,6-difluoro-4- (2- methoxyethoxy) benzyl]- 1H-indazol-3-yl}- N-(pyrimidin-4-yl)-6- (2,2,2-trifluoroethyl)- 6,7-dihydro-5H- pyrrolo[3,4- d]pyrimidin-4-amine | ¹H-NMR (400 MHz, DMSO- d₆): δ [ppm] = 3.26 (s, 3H), 3.61 (m, 2H), 3.63 (q, 2H), 4.12 (m, 2H), 4.22 (br. s., 2H), 4.28 (br. s., 2H), 5.72 (s, 2H), 6.86 (m, 2H), 7.30 (dd, 1H), 7.52 (dd, 1H), 7.88 (d, 1H), 8.50 (d, 1H), 8.56 (dd, 1H), 8.57 (d, 1H), 8.86 (br. s., 1H), 10.46 (br. s., 1H). |
| 2-20-19 SM = 2-9-28 1.5 eq. 2,2,2- trifluoro- ethyl trifluoro- methane- sulfonate, 16 h rt | 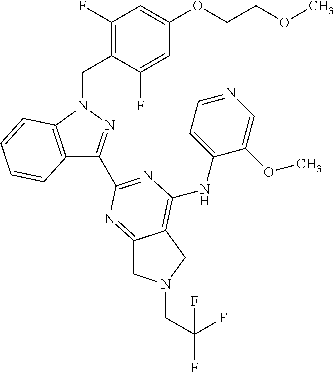 [SMILES: COCCOc1cc(F)c(Cn2nc(-c3nc4c(c(Nc5ccncc5OC)n3)CN(CC(F)(F)F)C4)c3ccccc32)c(F)c1] | 2-{1-[2,6-difluoro-4- (2- methoxyethoxy) benzyl]- 1H-indazol-3-yl}- N-(3- methoxypyridin-4- yl)-6-(2,2,2- trifluoroethyl)-6,7- dihydro-5H- pyrrolo[3,4- d]pyrimidin-4-amine | ¹H-NMR (400 MHz, DMSO- d₆): δ [ppm] = 3.26 (s, 3H), 3.62 (m, 2H), 3.64 (q, 2H), 3.94 (s, 3H), 4.12 (m, 2H), 4.19 (br. s., 2H), 4.24 (br. s., 2H), 5.70 (s, 2H), 6.83 (m, 2H), 7.19 (dd, 1H), 7.47 (dd, 1H), 7.83 (d, 1H), 8.18 (d, 1H), 8.32 (d, 1H), 8.33 (d, 1H), 8.38 (s, 1H), 8.49 (br. s., 1H). |

### Example 2-21-1

### Preparation of 2-[1-(4-ethoxy-2,6-difluorobenzyl)-1H-indazol-3-yl]-N-(6-methylpyrimidin-4-yl)-6,7-dihydro-5H-cyclopenta[d]pyrimidin-4-amine

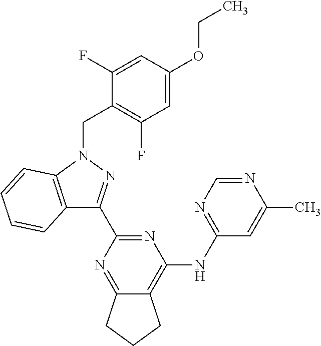 [SMILES: CCOc1cc(F)c(Cn2nc(-c3nc4c(c(Nc5cc(C)ncn5)n3)CCC4)c3ccccc32)c(F)c1]

150 mg (0.34 mmol) 3-(4-chloro-6,7-dihydro-5H-cyclopenta[d]pyrimidin-2-yl)-1-(4-ethoxy-2,6-difluorobenzyl)-1H-indazole, 41 mg (0.37 mmol) 6-methylpyrimidin-4-amine, 166 mg (0.51 mmol) caesium carbonate, 8 mg (0.03 mmol) palladium acetate and 39 mg (0.07 mmol) (rac)-2,2′-bis(diphenylphosphino)-1,1′-binaphthyl were dissolved under inert atmosphere in 1.58 mL N,N-dimethylformamide and 1.58 mL dioxane. The suspension was heated to 100° C. overnight, cooled to room temperature and extracted with dichloromethane and water. The organic layer was filtered through a silicon filter and concentrated under reduced pressure. The crude product was purified by HPLC to yield 46 mg (25% yield, purity 94%) 2-[1-(4-ethoxy-2,6-difluorobenzyl)-1H-indazol-3-yl]-N-(6-methylpyrimidin-4-yl)-6,7-dihydro-5H-cyclopenta[d]pyrimidin-4-amine.

¹H-NMR (400 MHz, DMSO-d₆): δ [ppm]=1.40 (t, 3H) 2.20-2.33 (m, 2H) 2.60 (s, 3H) 2.96 (t, 2H) 3.19 (t, 2H) 3.98 (q, 2H) 5.77 (s, 2H) 6.46 (s, 1H) 6.48 (s, 1H) 7.20 (s, 1H) 7.28 (t, 1H) 7.45 (ddd, 1H) 7.61 (d, 1H) 8.63 (d, 1H) 8.75 (d, 2H).

### Example 2-22-1

### Preparation of 2-[1-(4-ethoxy-2,6-difluorobenzyl)-1H-indazol-3-yl]-N-(3-methoxypyridin-4-yl)-5-[1-(3,3,3-trifluoropropyl)piperidin-4-yl]pyrimidin-4-amine

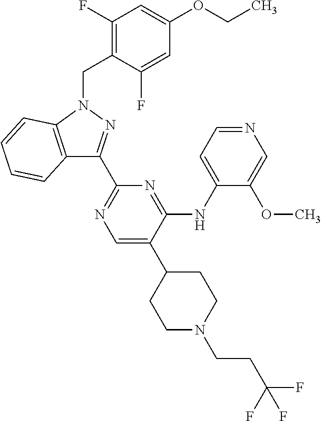 [SMILES: CCOc1cc(F)c(Cn2nc(-c3ncc(C4CCN(CCC(F)(F)F)CC4)c(Nc4ccncc4OC)n3)c3ccccc32)c(F)c1]

66 mg 2-17-5 2-[1-(4-ethoxy-2,6-difluorobenzyl)-1H-indazol-3-yl]-N-(3-methoxypyridin-4-yl)-5-(piperidin-4-yl)pyrimidin-4-amine (82% purity, 95.0 μmol, 1.0 eq.) was dissolved in 0.75 mL acetronitrile and 20 mg potassium carbonate (142 μmol, 1.5 eq.) was added. 12 1,1,1-trifluoro-3-iodopropane (100 μmol, 1.1 eq.) was added dropwise. The reaction mixture was stirred at 70° C. over night. Again 12 μL 1,1,1-trifluoro-3-iodopropane (100 μmol, 1.1 eq.) was added dropwise and stirring was continued over night at 70° C. Again 4 μL 1,1,1-trifluoro-3-iodopropane (23 μmol, 0.25 eq.) was added dropwise and stirring was continued over night at 70° C. The reaction mixture was diluted with water and ethyl acetate. The layers were separated and the aqueous layer was extracted with ethyl acetate twice. The combined organic layers were dried using a waterresistant filter an the filtrate was concentrated under reduced pressure. The crude product was purified by flash chromatography to provide the 97% pure target compound: 34.0 mg, 0.05 mmol, 52%.

¹H-NMR (400 MHz, DMSO-d₆): δ [ppm]=1.29 (t, 3H), 1.69-1.82 (m, 2H), 1.87-1.97 (m, 2H), 2.12-2.26 (m, 2H), 2.53-2.57 (m, 2H), 2.57-2.66 (m, 2H), 2.71-2.82 (m, 1H), 3.03-3.15 (m, 2H), 3.99 (s, 3H), 4.04 (q, 2H), 5.70 (s, 2H), 6.74-6.85 (m, 2H), 7.18-7.28 (m, 1H), 7.44-7.54 (m, 1H), 7.86 (d, 1H), 8.07 (s, 1H), 8.21 (d, 1H), 8.34 (d, 1H), 8.38 (s, 1H), 8.50 (s, 1H), 8.62 (d, 1H)

### REFERENCE COMPOUNDS

### Reference 3-1-1

### Preparation of 2-({2-[1-(4-ethoxy-2,6-difluorobenzyl)-1H-indazol-3-yl]-4-(pyridin-4-ylamino)pyrimidin-5-yl}oxy)ethanol

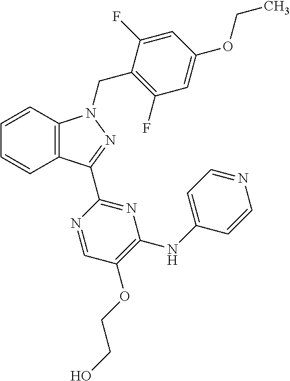 [SMILES: CCOc1cc(F)c(Cn2nc(-c3ncc(OCCO)c(Nc4ccncc4)n3)c3ccccc32)c(F)c1]

2-({2-[1-(4-ethoxy-2,6-difluorobenzyl)-1H-indazol-3-yl]-4-(pyridin-4-ylamino)pyrimidin-5-yl}oxy)ethanol was prepared according WO 2013050438 compound 13-1.

### Reference 3-2-1

### Preparation 2-[1-(4-ethoxy-2,6-difluorobenzyl)-1H-indazol-3-yl]-5-[2-(methylsulfinyl)ethoxy]-N-(pyridin-4-yl)pyrimidin-4-amine

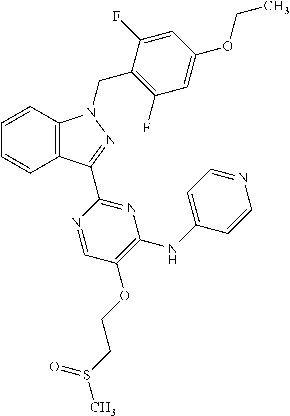 [SMILES: CCOc1cc(F)c(Cn2nc(-c3ncc(OCCS(C)=O)c(Nc4ccncc4)n3)c3ccccc32)c(F)c1]

2-[1-(4-ethoxy-2,6-difluorobenzyl)-1H-indazol-3-yl]-5-[2-(methylsulfinyl)ethoxy]-N-(pyridin-4-yl)pyrimidin-4-amine was prepared according WO 2013050438 compound 36-1.

### Reference 3-3-1

### Preparation 2-[1-(4-ethoxy-2,6-difluorobenzyl)-1H-indazol-3-yl]-5-[2-(methylsulfonyl)ethoxy]-N-(pyridin-4-yl)pyrimidin-4-amine

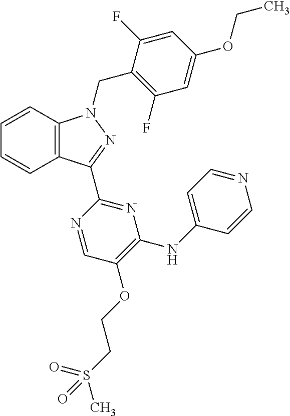 [SMILES: CCOc1cc(F)c(Cn2nc(-c3ncc(OCCS(C)(=O)=O)c(Nc4ccncc4)n3)c3ccccc32)c(F)c1]

2-[1-(4-ethoxy-2,6-difluorobenzyl)-1H-indazol-3-yl]-5-[2-(methylsulfonyl)ethoxy]-N-(pyridin-4-yl)pyrimidin-4-amine was prepared according WO 2013050438 compound 37-1.

### Reference 3-4-1

### Preparation 5-(2-aminoethoxy)-2-[1-(4-ethoxy-2,6-difluorobenzyl)-1H-indazol-3-yl]-N-(pyridin-4-yl)pyrimidin-4-amine

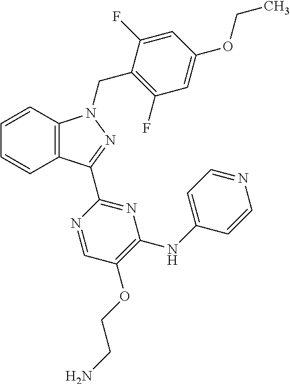 [SMILES: CCOc1cc(F)c(Cn2nc(-c3ncc(OCCN)c(Nc4ccncc4)n3)c3ccccc32)c(F)c1]

5-(2-aminoethoxy)-2-[1-(4-ethoxy-2,6-difluorobenzyl)-1H-indazol-3-yl]-N-(pyridin-4-yl)pyrimidin-4-amine was prepared according WO 2013050438 compound 38-1.

### Reference 3-5-1

### Preparation 1-(3,3-difluoroazetidin-1-yl)-2-({2-[1-(4-ethoxy-2,6-difluorobenzyl)-1H-indazol-3-yl]-4-(pyridin-4-ylamino)pyrimidin-5-yl}oxy)ethanone

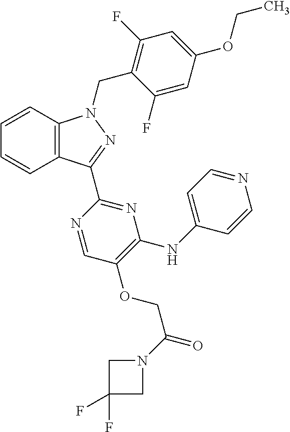 [SMILES: CCOc1cc(F)c(Cn2nc(-c3ncc(OCC(=O)N4CC(F)(F)C4)c(Nc4ccncc4)n3)c3ccccc32)c(F)c1]

1-(3,3-difluoroazetidin-1-yl)-2-({2-[1-(4-ethoxy-2,6-difluorobenzyl)-1H-indazol-3-yl]-4-(pyridin-4-ylamino)pyrimidin-5-yl}oxy)ethanone was prepared according WO 2013050438 compound 4-15.

Biological Investigations

The following assays can be used to illustrate the commercial utility of the compounds according to the present invention.

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values calculated utilizing data sets obtained from testing of one or more synthetic batch.

Biological Assay 1.0:

Bub1 Kinase Assay

Bub1-inhibitory activities of compounds described in the present invention were quantified using a time-resolved fluorescence energy transfer (TR-FRET) kinase assay which measures phosphorylation of the synthetic peptide Biotin-Ahx-VLLPKKSFAEPG (SEQ ID No. 1) (C-terminus in amide form), purchased from e.g. Biosyntan (Berlin, Germany) by the (recombinant) catalytic domain of human Bub1 (amino acids 704-1085), expressed in Hi5 insect cells with an N-terminal His6-tag and purified by affinity-(Ni-NTA) and size exclusion chromatography.

In a typical assay 11 different concentrations of each compound (0.1 nM, 0.33 nM, 1.1 nM, 3.8 nM, 13 nM, 44 nM, 0.15 μM, 0.51 μM, 1.7 μM, 5.9 μM and 20 μM) were tested in duplicate within the same microtiter plate. To this end, 100-fold concentrated compound solutions (in DMSO) were previously prepared by serial dilution (1:3.4) of 2 mM stocks in a clear low volume 384-well source microtiter plate (Greiner Bio-One, Frickenhausen, Germany), from which 50 nL of compounds were transferred into a black low volume test microtiter plate from the same supplier. Subsequently, 2 μL of Bub1 (the final concentration of Bub1 was adjusted depending on the activity of the enzyme lot in order to be within the linear dynamic range of the assay: typically ˜200 ng/mL were used) in aqueous assay buffer [50 mM Tris/HCl pH 7.5, 10 mM magnesium chloride (MgCl₂), 200 mM potassium chloride (KCl), 1.0 mM dithiothreitol (DTT), 0.1 mM sodium ortho-vanadate, 1% (v/v) glycerol, 0.01% (w/v) bovine serum albumine (BSA), 0.005% (v/v) Trition X-100 (Sigma), 1× Complete EDTA-free protease inhibitor mixture (Roche)] were added to the compounds in the test plate and the mixture was incubated for 15 min at 22° C. to allow pre-equilibration of the putative enzyme-inhibitor complexes before the start of the kinase reaction, which was initiated by the addition of 3 μL 1.67-fold concentrated solution (in assay buffer) of adenosine-tri-phosphate (ATP, 10 μM final concentration) and peptide substrate (1 μM final concentration). The resulting mixture (5 μL final volume) was incubated at 22° C. during 60 min., and the reaction was stopped by the addition of 5 μL of an aqueous EDTA-solution (50 mM EDTA, in 100 mM HEPES pH 7.5 and 0.2% (w/v) bovine serum albumin) which also contained the TR-FRET detection reagents (0.2 μM streptavidin-XL665 [Cisbio Bioassays, Codolet, France] and 1 nM anti-phosho-Serine antibody [Merck Millipore, cat. #35-001] and 0.4 nM LANCE EU-W1024 labeled anti-mouse IgG antibody [Perkin-Elmer, product no. AD0077, alternatively a Terbium-cryptate-labeled anti-mouse IgG antibody from Cisbio Bioassays can be used]). The stopped reaction mixture was further incubated 1 h at 22° C. in order to allow the formation of complexes between peptides and detection reagents. Subsequently, the amount of product was evaluated by measurement of the resonance energy transfer from the Eu-chelate-antibody complex recognizing the Phosphoserine residue to the streptavidin-XL665 bound to the biotin moiety of the peptide. To this end, the fluorescence emissions at 620 nm and 665 nm after excitation at 330-350 nm were measured in a TR-FRET plate reader, e.g. a Rubystar or Pherastar (both from BMG Labtechnologies, Offenburg, Germany) or a Viewlux (Perkin-Elmer) and the ratio of the emissions (665 nm/622 nm) was taken as indicator for the amount of phosphorylated substrate. The data were normalised using two sets of (typically 32-) control wells for high- (=enzyme reaction without inhibitor=0%=Minimum inhibition) and low- (=all assay components without enzyme=100%=Maximum inhibition) Bub1 activity. IC₅₀ values were calculated by fitting the normalized inhibition data to a 4-parameter logistic equation (Minimum, Maximum, IC50, Hill; Y=Max+(Min−Max)/(1+(X/IC50) Hill)).

Biological Assay 2.0:

Proliferation Assay:

Cultivated tumor cells (cells were ordered from ATCC, except HeLa-MaTu and HeLa-MaTu-ADR, which were ordered from EPO-GmbH, Berlin) were plated at a density of 1000 to 5000 cells/well, depending on the growth rate of the respective cell line, in a 96-well multititer plate in 200 μL of their respective growth medium supplemented 10% fetal calf serum. After 24 hours, the cells of one plate (zero-point plate) were stained with crystal violet (see below), while the medium of the other plates was replaced by fresh culture medium (200 μL), to which the test substances were added in various concentrations (0 μM, as well as in the range of 0.001-10 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μL measuring point of an 11% glutaric aldehyde solution for 15 minutes at room temperature. After three washing cycles of the fixed cells with water, the plates were dried at room temperature. The cells were stained by adding 100 μL/measuring point of a 0.1% crystal violet solution (pH 3.0). After three washing cycles of the stained cells with water, the plates were dried at room temperature. The dye was dissolved by adding 100 μl/measuring point of a 10% acetic acid solution. Absorbtion was determined by photometry at a wavelength of 595 nm. The change of cell number, in percent, was calculated by normalization of the measured values to the absorbtion values of the zero-point plate (=0%) and the absorbtion of the untreated (0 μm) cells (=100%). The IC₅₀ values were determined by means of a 4 parameter fit.

TABLE 1
Compounds had been evaluated in the following cell lines,
which examplify the sub-indications listed

| Tumor indication | Cell line |
|---|---|
| Cervical cancer | HeLa |
| | HeLa-MaTu-ADR |
| Non-small cell lung | NCI-H460 |
| cancer (NSCLC) | |
| Prostate cancer | DU145 |
| Colon cancer | Caco2 |
| Melanoma | B16F10 |

Biological Assay 3.0:

Proliferation Assay (HeLa+Paclitaxel):

Cultivated HeLa human cervical tumor cells (DSMZ ACC-57) were plated at a density of 3000 cells/well in a 96-well multititer plate in 200 μL of growth medium supplemented 10% fetal calf serum. After 24 hours, the cells of one plate (zero-point plate) were stained with crystal violet (see below). The medium of the other plates was supplemented with 3 nM of paclitaxel (Sigma-Aldrich) and the cells were incubated at 37° C. After 4 hours the test substances were added in various concentrations (0 μM, as well as in the range of 0.001-10 μM; the final concentration of the solvent dimethyl sulfoxide was adjusted to 0.1%) using a Hewlett-Packard HP D300 Digital Dispenser. The cells were incubated for another 92 hours at 37° C. in the presence of test substances. Cell proliferation was determined by staining the cells with crystal violet: the cells were fixed by adding 20 μL/measuring point of an 11% glutaric aldehyde solution for 15 minutes at room temperature. After three washing cycles of the fixed cells with water, the plates were dried at room temperature. The cells were stained by adding 100 μL/measuring point of a 0.1% crystal violet solution (pH 3.0). After three washing cycles of the stained cells with water, the plates were dried at room temperature. The dye was dissolved by adding 100 μL/measuring point of a 10% acetic acid solution. Absorbtion was determined by photometry at a wavelength of 595 nm. The change of cell number, in percent, was calculated by normalization of the measured values to the absorbtion values of the zero-point plate (=0%) and the absorbtion of the untreated (0 μm) cells (=100%). The IC₅₀ values were determined by means of a 4 parameter fit.

Biological Assay 4.1: Formation-Assay

Cell-Based Mechanistic Assay: Changes of Phosphorylation Status of Histone 2A by Inhibition of Kinase Activity of Bub1

This assay determines the suppression of histone 2A phosphorylation by a Bub1 kinase inhibitor during co-treatment with Nocodazole. 25000 cells (cells were ordered from ATCC) were seeded in 96 well plate for 5 h at 37° C. Cells were treated with Nocodazole (1 μg/ml) and varying concentrations (between 3 nM and 10 μM) of test compounds for 16 h. Cells were fixed (20 min, Fixing solution R&D), washed three times with PBS and blocked with Odyssey blocking buffer before incubating with the primary antibody against phosphorylated H2A (5 μg/ml ABIN482721) overnight at 2-8° C. After washing, secondary IRDye-labeled antibody mix with cell stains was added for 1 h and washed again with PBS. Plates were scanned with LiCor Odyssey Infrared Imager CLx at 800 nm for P-H2A and at 700 nm for cell stains Draq5/Sapphire. The quotient of 800 nm and 700 nm for Nocodazole only treated cells was set as 100% and the quotient of 800 nm and 700 nm of untreated cells was set as 0%. The results given as % reflecting the inhibition of Bub1 kinase activity compared to control and normalized according to cell number. The IC₅₀ values were determined by means of a 4 parameter fit.

Biological Assay 4.2: Abrogation-Assay

Cell-Based Mechanistic Assay: Changes of Phosphorylation Status of Pre-Induced Phospho-Histone 2A by Inhibition of Kinase Activity of Bub1

This assay measures the inhibition of histone 2A phosphorylation, which was induced by pre-treatment of the cells with Nocodazole, by a Bub1 kinase inhibitor. 25000 cells (cells were ordered from ATCC) were seeded in 96 well plate for 5 h at 37° C. Cells were treated with Nocodazole (1 μg/ml). After 16 h varying concentrations (between 3 nM and 10 μM) of test compounds were added and the cells were incubated for another 1 h. Cells were fixed (20 min, Fixing solution R&D), washed three times with PBS and blocked with Odyssey blocking buffer before incubating with the primary antibody against phosphorylated H2A (5 μg/ml ABIN482721) overnight at 2-8° C. After washing, secondary IRDye-labeled antibody mix with cell stains was added for 1 h and washed again with PBS. Plates were scanned with LiCor Odyssey Infrared Imager CLx at 800 nm for P-H2A and at 700 nm for cell stains Draq5/Sapphire. The quotient of 800 nm and 700 nm for Nocodazole only treated cells was set as 100% and the quotient of 800 nm and 700 nm of untreated cells was set as 0%. The results given as % reflecting the inhibition of Bub1 kinase activity compared to control and normalized according to cell number. The IC₅₀ values were determined by means of a 4 parameter fit.

Histone H2A is an immediate intracellular substrate of Bub1 kinase. Determination of the phosphorylation status of Histone H2A provides a direct measure of the intracellular activity of Bub1 kinase. The compounds according to the invention inhibit Bub1 kinase activity in with IC₅₀ values in the nanomolar range in biochemical assays similar as it was described for compounds from WO 2013050438. Surprisingly, it was now found that the compounds according to the invention inhibit intracellular Bub1 kinase activity, in terms of inhibition of Histone H2A phosphorylation, much more potently (IC₅₀ [M], range: 10⁻⁶ to 10⁻⁹) when compared to compounds from WO 2013050438. (IC₅₀ [nM], range: 10⁻⁶ to 10⁻⁷).

Biological Assay 5.0:

Evaluation of Drug-Drug Interaction Potential with Paclitaxel

To evaluate the drug-drug interaction potential of test compounds with paclitaxel in vivo 8 mg/kg of paclitaxel were injected once intravenously into the tail vein of NMRI nude mice. Immediately thereafter 50 mg/kg of the test compound was administered by gavage to mice. Blood was taken from mice following decapitation 1, 3, 7 and 24 hours after injection of Paclitaxel. Plasma concentrations of test compound and of paclitaxel, respectively, were determined by LC/MSMS. The data from the paclitaxel mono treatment group, the test compound mono treatment group, and the combination treatment group were compared for evaluation of the drug-drug interaction potential.

The following table gives the data for the examples of the present invention for the biological assays 1 and 2:

| | Biological Assay 1: | Biological Assay 2: |
|---|---|---|
| | Bub1 kinase assay | Proliferation assay (HeLa cell line) |
| | median IC₅₀ [mol/l] | median IC₅₀ [mol] |
| Example No. | | |
| 2-1-1 | 5.0E−09 | 3.6E−06 |
| 2-1-2 | 5.6E−09 | 7.3E−06 |
| 2-2-1 | 1.1E−08 | nd |
| 2-2-2 | 1.2E−08 | nd |
| 2-2-3 | nd | nd |
| 2-2-4 | 1.3E−08 | nd |
| 2-2-8 | 1.0E−08 | nd |
| 2-2-9 | 1.2E−08 | nd |
| 2-2-10 | 2.0E−08 | nd |
| 2-2-11 | 1.7E−08 | nd |
| 2-2-12 | 1.2E−08 | 2.3E−06 |
| 2-2-13 | 1.5E−08 | nd |
| 2-2-14 | 1.3E−07 | 7.8E−06 |
| 2-2-15 | 1.4E−08 | nd |
| 2-2-16 | 1.5E−08 | 6.9E−06 |
| 2-2-17 | 9.5E−08 | 9.3E−06 |
| 2-2-18 | 1.3E−07 | >1.0E−05 |
| 2-2-19 | 2.8E−08 | 5.4E−06 |
| 2-2-20 | 5.8E−08 | nd |
| 2-2-47 | 8.7E−9 | nd |
| 2-2-48 | 3.7E−9 | nd |
| 2-3-1 | 6.1E−09 | 3.5E−06 |
| 2-3-2 | 9.0E−09 | 2.8E−06 |
| 2-3-3 | 8.9E−09 | nd |
| 2-3-4 | 5.9E−09 | 2.1E−6 |
| 2-4-1 | 1.2E−08 | >1.0E−5 |
| 2-4-2 | 1.5E−08 | >1.0E−05 |
| 2-4-3 | 9.4E−07 | >1.0E−05 |
| 2-4-4 | 4.8E−07 | nd |
| 2-4-5 | 2.8E−08 | 2.6E−6 |
| 2-4-6 | 9.9E−09 | 2.1E−6 |
| 2-4-7 | 9.7E−09 | nd |
| 2-4-8 | 5.0E−09 | 1.8E−06 |
| 2-4-9 | 1.1E−08 | nd |
| 2-4-10 | 1.8E−08 | >1.0E−5 |
| 2-4-11 | 8.8E−08 | >1.0E−05 |
| 2-4-12 | 4.8E−08 | 1.1E−06 |
| 2-6-4 | 9.1E−09 | 1.4E−06 |
| 2-6-5 | 7.7E−09 | nd |
| 2-6-7 | 9.3E−09 | nd |
| 2-6-8 | 4.6E−08 | nd |
| 2-6-11 | 1.4E−08 | nd |
| 2-6-12 | 4.4E−8 | >1.0E−05 |
| 2-6-13 | 5.3E−8 | 9.7E−6 |
| 2-6-14 | 3.6E−8 | 2.4E−6 |
| 2-6-15 | 3.1E−8 | 2.2E−6 |
| 2-6-16 | 5.5E−7 | >1.0E−05 |
| 2-6-17 | 5.8E−8 | 6.8E−6 |
| 2-6-18 | 3.0E−8 | >1.0E−05 |
| 2-6-19 | 8.9E−9 | >1.0E−05 |
| 2-6-20 | 6.6E−8 | 3.4E−6 |
| 2-6-21 | 1.1E−8 | 3.4E−6 |
| 2-6-22 | 8.1E−8 | 9.2E−6 |
| 2-6-23 | 8.2E−8 | 3.0E−6 |
| 2-6-24 | 1.2E−7 | >1.0E−05 |
| 2-6-25 | 4.8E−8 | 7.6E−6 |
| 2-6-26 | 4.2E−8 | nd |
| 2-6-27 | 4.3E−8 | 5.1E−6 |
| 2-6-28 | 5.1E−7 | nd |
| 2-6-29 | 3.1E−8 | 1.3E−6 |
| 2-6-30 | 3.2E−6 | nd |
| 2-6-31 | 6.7E−9 | 4.9E−6 |
| 2-6-32 | 2.1E−7 | nd |
| 2-6-33 | 5.7E−9 | 3.5E−6 |
| 2-6-34 | 5.1E−9 | 3.4E−6 |
| 2-6-35 | 5.4E−9 | 2.3E−6 |
| 2-6-36 | 9.6E−9 | 3.4E−6 |
| 2-6-37 | 4.0E−8 | 9.5E−6 |
| 2-6-38 | 1.0E−8 | >1.0E−05 |
| 2-6-39 | 5.5E−8 | 7.0E−6 |
| 2-6-40 | 9.3E−9 | nd |
| 2-6-41 | 1.4E−8 | nd |
| 2-6-42 | 1.4E−8 | nd |
| 2-6-43 | 8.4E−8 | nd |
| 2-6-44 | 1.2E−8 | nd |
| 2-7-1 | 6.3E−09 | 3.5E−06 |
| 2-9-1 | 9.4E−09 | 1.3E−06 |
| 2-9-2 | 8.8E−09 | 1.1E−06 |
| 2-9-3 | 5.8E−09 | 1.1E−06 |
| 2-9-4 | 7.8E−09 | 1.9E−06 |
| 2-9-5 | 1.4E−08 | 1.8E−06 |
| 2-9-6 | 9.0E−08 | 3.7E−06 |
| 2-9-7 | 7.4E−09 | 1.0E−05 |
| 2-9-8 | 4.4E−07 | 1.8E−06 |
| 2-9-9 | 5.2E−08 | 2.3E−06 |
| 2-9-10 | 7.1E−09 | 1.0E−06 |
| 2-9-11 | 3.6E−07 | 1.1E−06 |
| 2-9-12 | 4.3E−09 | nd |
| 2-9-13 | 7.1E−09 | 1.0E−06 |
| 2-9-14 | 1.1E−08 | 4.3E−06 |
| 2-9-15 | 3.3E−06 | 4.0E−06 |
| 2-9-16 | 1.6E−08 | nd |
| 2-9-17 | nd | 2.1E−06 |
| 2-9-18 | 1.5E−08 | 5.5E−06 |
| 2-9-19 | 4.4E−8 | nd |
| 2-9-20 | 4.9E−8 | nd |
| 2-9-21 | 1.3E−8 | nd |
| 2-9-22 | 9.3E−9 | nd |
| 2-9-24 | 3.8E−8 | nd |
| 2-9-25 | 7.7E−8 | nd |
| 2-9-26 | 1.8E−7 | nd |
| 2-9-27 | 2.0E−8 | nd |
| 2-9-28 | | nd |
| 2-10-1 | 1.1E−08 | >1.0E−05 |
| 2-10-2 | 5.5E−09 | nd |
| 2-11-1 | 6.2E−08 | >1.0E−05 |
| 2-11-2 | 2.0E−08 | 3.4E−06 |
| 2-11-3 | 1.2E−08 | 3.0E−06 |
| 2-11-4 | 2.1E−7 | nd |
| 2-11-5 | 2.3E−8 | 1.7E−6 |
| 2-12-1 | 8.4E−09 | nd |
| 2-12-2 | 1.2E−08 | 2.1E−06 |
| 2-12-3 | 1.7E−08 | 4.9E−06 |
| 2-12-4 | 5.2E−09 | 1.8E−06 |
| 2-12-5 | 6.5E−09 | 1.3E−06 |
| 2-12-6 | 1.1E−08 | 1.2E−06 |
| 2-12-8 | 1.6E−08 | nd |
| 2-12-9 | 2.1E−08 | 3.7E−06 |
| 2-12-12 | 6.8E−09 | 3.6E−06 |
| 2-12-13 | 6.0E−09 | 3.4E−06 |
| 2-12-14 | 8.8E−09 | 1.8E−06 |
| 2-12-15 | 6.8E−08 | nd |
| 2-12-16 | 2.9E−07 | 1.0E−06 |
| 2-12-17 | nd | nd |
| 2-12-18 | 8.4E−09 | 2.0E−06 |
| 2-12-19 | nd | nd |
| 2-12-20 | 5.3E−09 | 2.8E−06 |
| 2-12-21 | nd | >1.0E−05 |
| 2-12-22 | 9.3E−09 | 1.2E−06 |
| 2-12-23 | 2.3E−08 | >1.0E−05 |
| 2-12-24 | 5.5E−08 | nd |
| 2-12-26 | 1.3E−07 | 8.0E−6 |
| 2-12-27 | 6.1E−07 | 3.1E−6 |
| 2-12-28 | 9.4E−09 | 5.3E−6 |
| 2-12-30 | 6.0E−08 | 8.6E−06 |
| 2-12-31 | 1.3E−08 | 3.9E−06 |
| 2-12-32 | 3.3E−07 | >1.0E−05 |
| 2-12-33 | 5.2E−07 | 8.3E−06 |
| 2-12-34 | 3.6E−08 | 7.8E−06 |
| 2-12-35 | 1.7E−08 | 1.0E−05 |
| 2-12-36 | 5.7E−8 | nd |
| 2-12-37 | 1.7E−8 | nd |
| 2-12-38 | 3.0E−6 | 8.8E−6 |
| 2-12-39 | 2.6E−08 | >1.0E−05 |
| 2-12-40 | 1.0E−8 | nd |
| 2-12-41 | 8.8E−09 | 3.5E−6 |
| 2-12-42 | 1.6E−08 | 3.4E−6 |
| 2-12-43 | 9.6E−09 | 3.6E−6 |
| 2-12-44 | 3.2E−07 | 9.6E−06 |
| 2-12-45 | 7.6E−07 | >1.0E−05 |
| 2-12-46 | 1.1E−06 | nd |
| 2-12-47 | 5.2E−09 | 6.1E−6 |
| 2-12-48 | 2.1E−08 | 1.2E−06 |
| 2-12-49 | 8.8E−09 | nd |
| 2-12-50 | 5.4E−09 | 9.7E−7 |
| 2-12-51 | 4.5E−08 | 4.1E−7 |
| 2-12-52 | 1.0E−07 | 1.1E−06 |
| 2-12-53 | 6.6E−07 | >1.0E−05 |
| 2-12-54 | 3.1E−08 | 3.3E−06 |
| 2-12-55 | 4.4E−8 | nd |
| 2-12-56 | 7.0E−9 | nd |
| 2-12-57 | 1.3E−7 | nd |
| 2-12-58 | 1.7E−8 | nd |
| 2-12-59 | 2.2E−8 | >1.0E−05 |
| 2-12-60 | 3.8E−8 | >1.0E−05 |
| 2-12-61 | 7.4E−8 | >1.0E−05 |
| 2-12-65 | 2.2E−8 | >1.0E−05 |
| 2-12-66 | 9.8E−8 | >1.0E−05 |
| 2-12-67 | 6.6E−8 | 4.4E−6 |
| 2-12-68 | 3.5E−8 | 2.7E−6 |
| 2-12-69 | 8.8E−9 | 8.5E−6 |
| 2-12-70 | 9.9E−9 | nd |
| 2-12-71 | 7.5E−8 | nd |
| 2-12-72 | 6.7E−7 | nd |
| 2-12-73 | 1.5E−8 | nd |
| 2-12-74 | 1.8E−7 | nd |
| 2-12-75 | 1.8E−7 | nd |
| 2-12-76 | 6.2E−9 | nd |
| 2-12-77 | 6.7E−9 | nd |
| 2-12-78 | 1.2E−8 | nd |
| 2-12-79 | 5.6E−9 | >1.0E−05 |
| 2-12-80 | 5.1E−8 | nd |
| 2-12-81 | 1.9E−8 | 1.6E−6 |
| 2-12-82 | >2.0E−5 | nd |
| 2-12-83 | >2.0E−5 | >1.0E−05 |
| 2-12-84 | 2.3E−6 | nd |
| 2-12-85 | 6.0E−6 | nd |
| 2-12-86 | 7.5E−9 | 3.9E−6 |
| 2-12-87 | 3.7E−8 | 9.3E−6 |
| 2-12-88 | 5.8E−9 | 1.2E−6 |
| 2-12-89 | 1.1E−6 | nd |
| 2-12-90 | 1.1E−8 | nd |
| 2-12-91 | nd | nd |
| 2-12-92 | 2.0E−8 | 2.7E−6 |
| 2-12-93 | 3.0E−8 | >1.0E−05 |
| 2-12-94 | 9.7E−9 | 3.9E−6 |
| 2-12-95 | 2.6E−8 | >1.0E−05 |
| 2-12-96 | >2.0E−5 | nd |
| | 9.2E−6″ | |
| 2-12-99 | 2.6E−8 | nd |
| 2-12-100 | 9.9E−8 | nd |
| 2-13-1 | 5.3E−09 | 3.7E−06 |
| 2-14-1 | 9.6E−09 | 1.0E−05 |
| 2-14-2 | 8.0E−09 | 1.1E−06 |
| 2-14-3 | 5.2E−09 | 3.0E−06 |
| 2-14-4 | 2.9E−09 | 9.4E−07 |
| 2-14-5 | 1.6E−08 | 3.6E−06 |
| 2-14-6 | 3.7E−08 | 3.6E−06 |
| 2-14-7 | 9.7E−09 | nd |
| 2-14-8 | 8.2E−9 | nd |
| 2-14-9 | 6.6E−08 | >1.0E−05 |
| 2-14-10 | 7.0E−09 | nd |
| 2-15-1 | 8.6E−09 | >1.0E−05 |
| 2-15-2 | 4.7E−08 | >1.0E−05 |
| 2-15-7 | 7.7E−09 | 3.5E−06 |
| 2-15-9 | 8.1E−8 | nd |
| 2-15-10 | 1.4E−6 | nd |
| 2-15-11 | 1.7E−6 | nd |
| 2-15-12 | 2.1E−8 | nd |
| 2-15-13 | 5.9E−8 | nd |
| 2-16-1 | 1.8E−08 | 3.0E−6 |
| 2-17-1 | 6.1E−09 | 1.1E−06 |
| 2-17-2 | 8.6E−09 | 3.1E−06 |
| 2-17-3 | nd | nd |
| 2-17-4 | nd | nd |
| 2-17-5 | | nd |
| 2-17-6 | | nd |
| 2-17-7 | | nd |
| 2-17-8 | 6.3E−9 | nd |
| 2-17-9 | 7.5E−9 | nd |
| 2-17-10 | 8.5E−8 | nd |
| 2-18-1 | 6.1E−09 | 1.7E−06 |
| 2-19-1 | 1.4E−08 | 1.2E−06 |
| 2-19-2 | 2.5E−08 | nd |
| 2-19-3 | 8.9E−09 | 2.1E−06 |
| 2-19-4 | 6.8E−08 | 1.2E−06 |
| 2-19-5 | 1.4E−07 | nd |
| 2-19-6 | nd | >1.0E−05 |
| 2-19-7 | 5.7E−08 | >1.0E−05 |
| 2-19-8 | 8.1E−09 | >1.0E−05 |
| 2-19-9 | 2.3E−08 | 1.3E−06 |
| 2-19-10 | 9.4E−08 | nd |
| 2-19-11 | 9.1E−09 | 1.8E−06 |
| 2-19-12 | 1.1E−08 | 4.0E−06 |
| 2-19-13 | 7.7E−09 | 3.4E−06 |
| 2-19-14 | 4.6E−07 | nd |
| 2-19-16 | 1.0E−07 | nd |
| 2-19-17 | 1.8E−08 | 1.5E−06 |
| 2-19-18 | 8.4E−08 | nd |
| 2-19-19 | 1.9E−08 | >1.0E−05 |
| 2-19-20 | 7.0E−09 | 5.2E−06 |
| 2-19-21 | 1.2E−08 | nd |
| 2-20-1 | 1.7E−08 | >1.0E−05 |
| 2-20-2 | 5.1E−8 | >1.0E−05 |
| 2-20-3 | 2.6E−8 | >1.0E−05 |
| 2-20-4 | 7.0E−8 | 1.1E−6 |
| 2-20-5 | 6.8E−8 | 3.6E−6 |
| 2-20-6 | 2.6E−7 | 3.2E−6 |
| 2-20-7 | 6.6E−8 | nd |
| 2-20-8 | >2.0E−5 | nd |
| 2-20-9 | 2.2E−7 | nd |
| 2-20-10 | 2.9E−7 | nd |
| 2-20-11 | 6.3E−8 | nd |
| 2-20-12 | 3.1E−8 | 1.2E−6 |
| 2-20-13 | 5.6E−7 | nd |
| 2-20-14 | 4.8E−8 | >1.0E−05 |
| 2-20-15 | 2.2E−8 | nd |
| 2-20-16 | 1.2E−7 | nd |
| 2-20-17 | 3.4E−8 | nd |
| 2-20-18 | 2.8E−8 | nd |
| 2-20-19 | nd | nd |
| 2-21-1 | 2.5E−08 | 6.0E−06 |
| 2-22-1 | 3.5E−8 | 3.6E−6 |
| Reference | | |
| Compounds: | | |
| 3-1-1 | 1.1E−08 | 1.5E−06 |
| 3-2-1 | 5.4E−09 | 4.5E−06 |
| 3-3-1 | 5.9E−09 | 4.1E−06 |
| 3-4-1 | 3.4E−09 | 1.4E−06 |
| 3-5-1 | 8.0E−09 | 3.9E−06 |

nd—not determined

Inhibition of proliferation of HeLa-MaTu-ADR, NCI-H460, DU145, Caco-2 and B16F10 cells by compounds according to the present invention is determined as described under Biological Assays 2.0. Inhibition of the proliferation of the forementioned cell lines by compounds according to the present invention is evidenced.

The following table gives the data for the examples of the present invention for the biological assays 3 and 4.1:

| | | Biological Assay 4.1: |
|---|---|---|
| | Biological Assay 3: | H2A Formation IC₅₀ |
| | HeLa + Paclitaxel IC₅₀ [mol/l] | [mol/l] |
| Example No. | | |
| 2-1-1 | 1.5E−7 | 1.4E−6 |
| 2-1-2 | 1.5E−7 | 2.9E−7 |
| 2-2-1 | nd | nd |
| 2-2-2 | nd | nd |
| 2-2-3 | nd | nd |
| 2-2-4 | nd | nd |
| 2-2-8 | nd | nd |
| 2-2-9 | nd | nd |
| 2-2-10 | nd | nd |
| 2-2-11 | nd | |
| 2-2-12 | 1.7E−7 | 8.0E−7 |
| 2-2-13 | nd | nd |
| 2-2-14 | 1.4E−7 | 3.9E−6 |
| 2-2-15 | nd | nd |
| 2-2-16 | 2.3E−7 | 1.9E−6 |
| 2-2-17 | 3.4E−7 | 1.2E−6 |
| 2-2-18 | 4.0E−7 | 8.7E−6 |
| 2-2-19 | 6.3E−7 | 1.8E−6 |
| 2-2-20 | nd | nd |
| 2-2-47 | nd | nd |
| 2-2-48 | nd | |
| 2-3-1 | 1.8E−7 | 2.3E−6 |
| | <1.0E−7 | |
| | 3.0E−7 | |
| 2-3-2 | 2.1E−7 | 9.9E−7 |
| | <1.0E−7 | |
| | 1.4E−7 | |
| 2-3-3 | nd | nd |
| 2-3-4 | 1.0E−6 | 5.2E−10 |
| 2-4-1 | 3.6E−6 | 9.6E−8 |
| 2-4-2 | 3.9E−6 | 7.0E−8 |
| 2-4-3 | >1.0E−5 | 1.0E−6 |
| 2-4-4 | nd | nd |
| 2-4-5 | 1.8E−7 | 3.9E−6 |
| 2-4-6 | nd | 1.3E−7 |
| 2-4-7 | nd | nd |
| 2-4-8 | 3.7E−7 | nd |
| 2-4-9 | nd | nd |
| 2-4-10 | 1.2E−6 | 1.0E−8 |
| 2-4-11 | 1.2E−7 | 1.9E−6 |
| 2-4-12 | 3.3E−6 | 2.3E−6 |
| 2-6-4 | 1.7E−7 | 5.2E−8 |
| 2-6-5 | nd | nd |
| 2-6-7 | nd | nd |
| 2-6-8 | nd | nd |
| 2-6-11 | nd | nd |
| 2-6-12 | 2.1E−6 | 2.3E−6 |
| 2-6-13 | 3.9E−7 | 8.4E−9 |
| 2-6-14 | 3.4E−7 | 7.1E−9 |
| 2-6-15 | 1.8E−7 | 1.9E−8 |
| 2-6-16 | 6.0E−8 | 5.6E−10 |
| 2-6-17 | 5.9E−8 | 6.0E−9 |
| 2-6-18 | 9.0E−8 | 7.9E−10 |
| 2-6-19 | 1.4E−7 | 2.6E−9 |
| 2-6-20 | 1.4E−7 | 1.8E−9 |
| 2-6-21 | 1.7E−7 | 8.4E−8 |
| 2-6-22 | 9.9E−8 | 1.8E−7 |
| 2-6-23 | 6.4E−8 | 1.2E−8 |
| 2-6-24 | 1.5E−7 | 1.4E−8 |
| 2-6-25 | 3.7E−7 | nd |
| 2-6-26 | nd | nd |
| 2-6-27 | 2.9E−7 | nd |
| 2-6-28 | nd | nd |
| 2-6-29 | 2.1E−7 | nd |
| 2-6-30 | nd | nd |
| 2-6-31 | 7.6E−7 | nd |
| 2-6-32 | nd | nd |
| 2-6-33 | 1.8E−6 | nd |
| 2-6-34 | 1.1E−6 | nd |
| 2-6-35 | 1.3E−6 | nd |
| 2-6-36 | 6.3E−7 | nd |
| 2-6-37 | 2.3E−6 | 2.6E−7 |
| 2-6-38 | 9.3E−7 | 2.3E−6 |
| 2-6-39 | 1.5E−6 | nd |
| 2-6-40 | nd | nd |
| 2-6-41 | nd | nd |
| 2-6-42 | nd | nd |
| 2-6-43 | nd | nd |
| 2-6-44 | nd | nd |
| 2-7-1 | 4.5E−7 | 5.7E−7 |
| 2-9-1 | <1.0E−7 | 1.7E−8 |
| | 8.6E−7 | |
| 2-9-2 | 3.6E−7 | 1.6E−6 |
| 2-9-3 | 2.4E−7 | >3.0E−6 |
| | | 4.9E−6 |
| 2-9-4 | 4.2E−7 | 4.4E−7 |
| 2-9-5 | 3.4E−7 | 1.8E−9 |
| 2-9-6 | 2.6E−7 | 2.9E−9 |
| 2-9-7 | 1.7E−7 | 6.7E−9 |
| 2-9-8 | 2.2E−7 | 1.4E−8 |
| 2-9-9 | 1.9E−7 | 2.7E−9 |
| 2-9-10 | 5.4E−8 | 1.5E−6 |
| 2-9-11 | 1.1E−7 | 2.8E−9 |
| 2-9-12 | nd | nd |
| 2-9-13 | 1.5E−7 | 1.6E−7 |
| 2-9-14 | 1.7E−7 | 1.1E−8 |
| 2-9-15 | 3.4E−7 | 2.8E−9 |
| 2-9-16 | nd | nd |
| 2-9-17 | 1.3E−7 | 2.0E−9 |
| 2-9-18 | 1.7E−7 | 6.7E−9 |
| 2-9-19 | nd | nd |
| 2-9-20 | nd | nd |
| 2-9-21 | nd | nd |
| 2-9-22 | nd | nd |
| 2-9-24 | nd | nd |
| 2-9-25 | nd | nd |
| 2-9-26 | nd | nd |
| 2-9-27 | nd | nd |
| 2-9-28 | nd | nd |
| 2-10-1 | 1.3E−6 | 3.4E−6 |
| 2-10-2 | nd | 1.4E−7 |
| 2-11-1 | 2.3E−7 | 4.1E−8 |
| 2-11-2 | 1.2E−7 | nd |
| 2-11-3 | 1.0E−7 | 9.3E−8 |
| 2-11-4 | nd | |
| 2-11-5 | 1.4E−7 | 8.3E−9 |
| 2-12-1 | nd | |
| 2-12-2 | 3.9E−7 | 1.8E−7 |
| 2-12-3 | 1.4E−6 | 3.4E−8 |
| 2-12-4 | 4.2E−7 | 8.9E−9 |
| 2-12-5 | 9.2E−7 | 3.9E−7 |
| 2-12-6 | 3.3E−7 | 6.0E−9 |
| 2-12-8 | nd | nd |
| 2-12-9 | 5.4E−7 | 1.1E−8 |
| 2-12-12 | <1.0E−7 | 3.6E−9 |
| 2-12-13 | 8.9E−8 | 6.6E−9 |
| 2-12-14 | 1.0E−7 | 1.1E−8 |
| 2-12-15 | nd | nd |
| 2-12-16 | 1.3E−7 | 5.7E−8 |
| 2-12-17 | nd | nd |
| 2-12-18 | 2.5E−7 | 4.1E−9 |
| 2-12-19 | nd | nd |
| 2-12-20 | 3.3E−7 | 2.9E−9 |
| 2-12-21 | 6.7E−7 | 3.8E−8 |
| 2-12-22 | 7.5E−7 | 3.5E−8 |
| 2-12-23 | 3.8E−7 | 1.0E−8 |
| 2-12-24 | nd | nd |
| 2-12-26 | 1.0E−6 | nd |
| 2-12-27 | 2.1E−7 | nd |
| 2-12-28 | 2.9E−7 | nd |
| 2-12-30 | 1.4E−7 | nd |
| 2-12-31 | 8.0E−8 | nd |
| 2-12-32 | 5.3E−7 | nd nd |
| 2-12-33 | 4.0E−7 | 3.7E−7 |
| 2-12-34 | 5.0E−7 | 7.0E−7 |
| 2-12-35 | 5.9E−7 | 1.4E−8 |
| 2-12-36 | nd | nd |
| 2-12-37 | nd | nd |
| 2-12-38 | 1.1E−7 | nd |
| 2-12-39 | 7.7E−7 | nd |
| 2-12-40 | nd | nd |
| 2-12-41 | 1.2E−7 | nd |
| 2-12-42 | 2.4E−7 | nd |
| 2-12-43 | 2.5E−7 | nd |
| 2-12-44 | 8.9E−8 | nd |
| 2-12-45 | 3.8E−7 | nd |
| 2-12-46 | nd | nd |
| 2-12-47 | 6.8E−8 | 2.1E−6 |
| 2-12-48 | 1.8E−7 | 1.1E−6 |
| 2-12-49 | nd | |
| 2-12-50 | 1.3E−6 | 2.3E−8 |
| 2-12-51 | 3.4E−7 | 5.7E−7 |
| 2-12-52 | 9.9E−7 | 2.5E−8 |
| 2-12-53 | 5.1E−7 | 1.4E−7 |
| 2-12-54 | 1.8E−7 | 1.4E−8 |
| 2-12-55 | nd | nd |
| 2-12-56 | nd | nd |
| 2-12-57 | nd | nd |
| 2-12-58 | nd | nd |
| 2-12-59 | 6.2E−8 | 1.2E−7 |
| 2-12-60 | 8.6E−8 | 3.9E−9 |
| 2-12-61 | 1.3E−7 | 2.4E−6 |
| 2-12-65 | 1.1E−7 | 3.3E−9 |
| 2-12-66 | 1.0E−7 | 2.2E−8 |
| 2-12-67 | 1.5E−7 | 2.2E−8 |
| 2-12-68 | 7.8E−8 | 3.8E−9 |
| 2-12-69 | 1.7E−7 | nd |
| 2-12-70 | nd | nd |
| 2-12-71 | nd | nd |
| 2-12-72 | nd | nd |
| 2-12-73 | nd | nd |
| 2-12-74 | nd | nd |
| 2-12-75 | nd | nd |
| 2-12-76 | nd | nd |
| 2-12-77 | nd | nd |
| 2-12-78 | nd | nd |
| 2-12-79 | 1.9E−6 | nd |
| 2-12-80 | nd | nd |
| 2-12-81 | 2.2E−7 | 1.3E−8 |
| 2-12-82 | nd | 3.0E−8 |
| 2-12-83 | 3.5E−7 | 3.1E−8 |
| 2-12-84 | nd | nd |
| 2-12-85 | nd | nd |
| 2-12-86 | 1.4E−6 | nd |
| 2-12-87 | 1.5E−6 | nd |
| 2-12-88 | 2.0E−7 | nd |
| 2-12-89 | nd | nd |
| 2-12-90 | nd | nd |
| 2-12-91 | nd | nd |
| 2-12-92 | 2.8E−7 | nd |
| 2-12-93 | >1.0E−5 | nd |
| 2-12-94 | 2.3E−6 | nd |
| 2-12-95 | 9.8E−6 | nd |
| 2-12-96 | nd | nd |
| 2-12-99 | nd | nd |
| 2-12-100 | nd | nd |
| 2-13-1 | 3.7E−7 | 8.2E−9 |
| 2-14-1 | 6.8E−7 | 2.8E−9 |
| 2-14-2 | 1.2E−7 | 1.6E−9 |
| 2-14-3 | 2.5E−7 | >3.0E−6 |
| | | 9.7E−8 |
| 2-14-4 | 1.2E−7 | 1.3E−8 |
| 2-14-5 | 5.4E−7 | 2.7E−9 |
| 2-14-6 | 2.7E−7 | 2.7E−9 |
| 2-14-7 | nd | nd |
| 2-14-8 | nd | nd |
| 2-14-9 | >1.0E−5 | nd |
| 2-14-10 | | nd |
| 2-15-1 | 6.9E−8 | nd |
| 2-15-2 | 4.5E−8 | 3.9E−8 |
| 2-15-7 | 1.5E−7 | >3.0E−6 |
| | | 9.7E−8 |
| 2-15-9 | nd | nd |
| 2-15-10 | nd | nd |
| 2-15-11 | nd | nd |
| 2-15-12 | nd | nd |
| 2-15-13 | nd | nd |
| 2-16-1 | 2.0E−7 | 1.8E−8 |
| 2-17-1 | 1.7E−7 | 1.2E−8 |
| 2-17-2 | 1.9E−7 | 4.8E−9 |
| 2-17-3 | | nd |
| 2-17-4 | nd | nd |
| 2-17-5 | | nd |
| 2-17-6 | | nd |
| 2-17-7 | | nd |
| 2-17-8 | nd | nd |
| 2-17-9 | nd | nd |
| 2-17-10 | nd | nd |
| 2-18-1 | 1.6E−7 | 2.0E−8 |
| 2-19-1 | 3.5E−7 | 3.5E−7 |
| 2-19-2 | nd | nd |
| 2-19-3 | 6.1E−7 | 3.4E−7 |
| 2-19-4 | 1.3E−7 | 4.5E−8 |
| 2-19-5 | nd | nd |
| 2-19-6 | 4.8E−7 | !6.0E−6 |
| 2-19-7 | 5.4E−7 | 2.1E−8 |
| 2-19-8 | 4.4E−7 | 2.5E−6 |
| 2-19-9 | 7.5E−8 | 1.8E−8 |
| 2-19-10 | nd | nd |
| 2-19-11 | 1.3E−7 | 1.8E−7 |
| 2-19-12 | 2.5E−7 | 6.3E−8 |
| 2-19-13 | 1.3E−7 | 3.8E−6 |
| 2-19-14 | nd | nd |
| 2-19-16 | nd | nd |
| 2-19-17 | 1.4E−7 | 7.1E−6 |
| 2-19-18 | nd | nd |
| 2-19-19 | 6.9E−7 | 7.3E−9 |
| 2-19-20 | 1.2E−7 | 4.9E−8 |
| 2-19-21 | nd | nd |
| 2-20-1 | 1.3E−7 | 1.1E−7 |
| 2-20-2 | 2.6E−7 | 9.1E−9 |
| 2-20-3 | 9.8E−7 | 2.2E−9 |
| 2-20-4 | 6.6E−8 | 3.1E−8 |
| 2-20-5 | 2.1E−7 | 5.0E−10 |
| 2-20-6 | 1.3E−7 | 8.1E−8 |
| 2-20-7 | nd | nd |
| 2-20-8 | nd | nd |
| 2-20-9 | nd | nd |
| 2-20-10 | nd | nd |
| 2-20-11 | | |
| 2-20-12 | 6.8E−8 | nd |
| 2-20-13 | nd | nd |
| 2-20-14 | 1.2E−7 | 2.6E−9 |
| 2-20-15 | nd | nd |
| 2-20-16 | nd | nd |
| 2-20-17 | nd | nd |
| 2-20-18 | nd | nd |
| 2-20-19 | | nd |
| 2-21-1 | 9.8E−8 | 1.9E−6 |
| 2-22-1 | 1.4E−7 | 7.2E−8 |
| Reference | | |
| Compounds: | | |
| 3-1-1 | 1.4E−6 | 1.1E−6 |
| 3-2-1 | 4.1E−7 | 1.9E−7 |
| 3-3-1 | 1.1E−6 | 5.0E−7 |
| 3-4-1 | nd | nd |
| 3-5-1 | nd | 3.6E−7 |

1. A compound of formula (I) 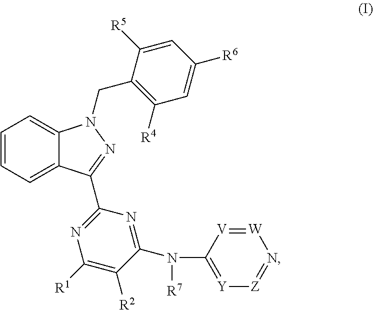
in which
V, W, Y and Z independently of each other represent CH or CR³,
or,
V represents N, and W, Y and Z independently of each other represent CH or CR³,
or,
W represents N, and V, Y and Z independently of each other represent CH or CR³,
or,
V and Y represent N, and W and Z independently of each other represent CH or CR³,
R¹ and R² represent, independently of each other, hydrogen, halogen, or a group selected from:
C₁-C₆-alkyl, C₃-C₆-cycloalkyl, C₁-C₆-haloalkyl, C₁-C₆-hydroxyalkyl,
(C₁-C₃-alkoxy)-(C₁-C₃-alkyl)-, —(C₁-C₆-alkyl)-S—R¹⁴, —(C₁-C₆-alkyl)-S(═O)—R¹⁴,
—(C₁-C₆-alkyl)-S(═O)₂—R¹⁴, —(C₁-C₆-alkyl)-S(═O)(═NR¹⁵)R¹⁴,
—(C₁-C₆-alkyl)-N(R¹¹)R¹², —(C₁-C₆-alkyl)-N(R¹⁷)R¹⁸, 5- or 6-membered heteroaryl,
a 5- to 7-membered oxygen containing heterocycloalkyl group,
(1,3-dioxolan-2-yl)-(C₁-C₆-alkyl)-, (1,3-dioxan-2-yl)-(C₁-C₆-alkyl)-, an azetidinyl group,
azetidinyl-(C₁-C₆-alkyl)-, a 5- to 7-membered heterocycloalkyl group, and
(5- to 7-membered heterocycloalkyl)-(C₁-C₆-alkyl)-,
in which 5- or 6-membered heteroaryl is connected to rest of the molecule via a carbon atom of the heteroaryl group,
in which 5- to 7-membered oxygen containing heterocycloalkyl is connected to rest of the molecule via a carbon atom of the 5- to 7-membered oxygen containing heterocycloalkyl group,
in which azetidinyl and 5- to 7-membered heterocycloalkyl are connected to the rest of the molecule via a carbon atom of the azetidinyl ring or via a carbon atom of the 5- to 7-membered heterocycloalkyl ring,
wherein 5- or 6-membered heteroaryl is optionally substituted, one or two times, with a substituent selected from: hydroxy, a halogen atom, cyano, C₁-C₄-alkyl, C₁-C₄-haloalkyl, C₁-C₄-alkoxy, and C₁-C₄-haloalkoxy group,
wherein azetidinyl is optionally substituted with a substituent selected from:
hydroxy, a halogen atom, cyano, C₁-C₄-alkyl, C₁-C₄-haloalkyl,
C₁-C₄-alkoxy, C₁-C₄-haloalkoxy, (C₁-C₃-alkoxy)-(C₁-C₄-alkyl)-,
C₃-C₆-cycloalkyl, C₃-C₆-cycloalkyloxy, —N(R¹¹)R¹², and
—N(H)C(═O)—(C₁-C₃-alkyl),
or being substituted with two halogen atoms,
wherein 5- to 7-membered heterocycloalkyl is optionally substituted, one, two, three, four or five times, identically or differently, with a substituent selected from:
hydroxy, halogen, cyano, C₁-C₄-alkyl, C₁-C₄-haloalkyl, C₁-C₄-alkoxy,
C₁-C₄-haloalkoxy, (C₁-C₃-alkoxy)-(C₁-C₄-alkyl)-, C₃-C₆-cycloalkyl,
(C₃-C₄-cycloalkyl)methyl-C₃-C₆-cycloalkyloxy, —N(R¹¹)R¹², C(═O)OH and
—N(H)C(═O)—(C₁-C₃-alkyl),
wherein at least one of R¹ and R² represents a group which connects via a carbon atom of said group to the rest of the molecule,
or,
R¹ and R² together represent a group, which is selected from: 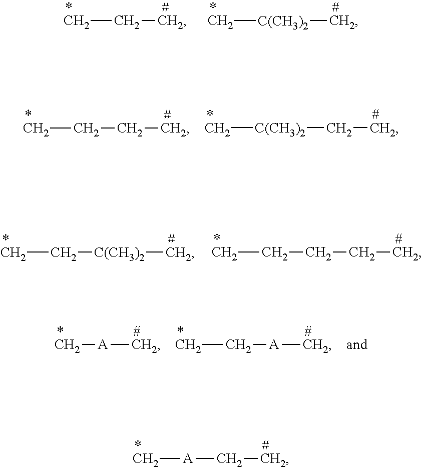
wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and # indicates the point of attachment of R² to the rest of the molecule,
R³ represents, independently of each other, halogen or a group selected from:
hydroxy, C₁-C₃-alkyl, C₃-C₄-cycloalkyl, C₁-C₃-haloalkyl, C₁-C₃-alkoxy,
C₁-C₃-haloalkoxy, —N(H)C(═O)H, —N(H)C(═O)—(C₁-C₃-alkyl),
—N(H)C(═O)—(C₁-C₃-alkyl)-(C₁-C₃-alkoxy), —N(H)C(═O)-phenyl,
—N(H)C(═O)—(C₃-C₄-cycloalkyl), —N(H)C(═O)—(C₁-C₃-hydroxyalkyl), —C(═O)OH,
—C(═O)NH₂, —S(═O)₂—(C₁-C₃-alkyl), —N(H)C(═O)—(C₁-C₃-alkyl)-(C₃-C₄-cycloalkyl), and —N(H)C(═O)N(H)R¹⁹,
said —N(H)C(═O)-phenyl being optionally substituted at the phenyl ring, one, two or three times, identically or differently, with a substituent selected from:
halogen, hydroxy, cyano, C₁-C₄-alkyl, C₁-C₄-haloalkyl, C₁-C₄-alkoxy,
C₁-C₄-haloalkoxy, C₃-C₆-cycloalkyl, and C₃-C₆-cycloalkyloxy,
said —N(H)C(═O)—(C₃-C₄-cycloalkyl) being optionally substituted at the C₃-C₄-cycloalkyl ring with a substituent selected from:
fluorine, chlorine, trifluoromethyl, and methoxy,
R⁴ and R⁵ represent, independently of each other, hydrogen or fluorine,
R⁶ represents a group selected from:
hydroxy, C₁-C₆-alkyl, C₁-C₆-haloalkyl, C₁-C₆-hydroxyalkyl,
(C₁-C₃-alkoxy)-(C₁-C₆-alkyl)-, C₃-C₆-cycloalkyl, (C₃-C₆-cycloalkyl)-(C₁-C₃-alkyl)-, C₁-C₆-alkoxy, C₁-C₆-haloalkoxy, (C₂-C₆-hydroxyalkyl)-O—,
(C₁-C₃-alkoxy)-(C₂-C₆-alkoxy)-, C₃-C₆-cycloalkyloxy,
(C₃-C₆-cycloalkyl)-(C₁-C₃-alkoxy)-, and R⁸,
wherein said hydroxyalkyl groups are optionally substituted with one, two or three halogen atoms selected from:
fluorine, and chlorine,
R⁷ represents hydrogen, or a 2-hydroxyethyl group,
or,
R² and R⁷ together represent a 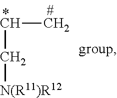
wherein * indicates the point of attachment of R² to the rest of the molecule,
and # indicates the point of attachment of R⁷ to the rest of the molecule,
R⁸ represents a group selected from:
—(C₂-C₆-alkyl)-OC(═O)—C(H)(R⁹)—N(H)C(═O)—C(H)(R¹⁰)—NH₂, and
—O—(C₂-C₆-alkyl)-OC(═O)—C(H)(R⁹)—N(H)C(═O)—C(H)(R¹⁰)—NH₂,
wherein said C₂-C₆-alkyl groups are optionally substituted with one, two or three halogen atoms selected from:
fluorine, and chlorine,
R⁹ and R¹⁰ independently of each other represent hydrogen (glycine) or a group selected from:
CH₃ (alanine), C(H)(CH₃)₂ (valine), (CH₂)₂CH₃ (norvaline), CH₂C(H)(CH₃)₂ (leucine),
C(H)(CH₃)CH₂CH₃ (isoleucine), (CH₂)₃CH₃ (norleucine), C(CH₃)₃ (2-tert-butylglycine),
benzyl (phenylalanine), 4-hydroxybenzyl (tyrosine), (CH₂)₃NH₂ (ornithine), (CH₂)₄NH₂ (lysine), (CH₂)₂C(H)(OH)CH₂NH₂ (hydroxylysine), CH₂OH (serine), (CH₂)₂OH (homoserine), C(H)(OH)CH₃ (threonine), (CH₂)₃N(H)C(═NH)NH₂ (arginine),
(CH₂)₃N(H)C(═O)NH₂ (citrulline), CH₂C(═O)NH₂ (asparagine), CH₂C(═O)OH (aspartic acid), (CH₂)₂C(═O)OH (glutamic acid), (CH₂)₂C(═O)NH₂ (glutamine), CH₂SH (cysteine),
(CH₂)₂SH (homocysteine), (CH₂)₂SCH₃ (methionine), CH₂SCH₃ (S-methylcysteine), (1H-imidazol-4-yl)methyl-(histidine), (1H-indol-3-yl)methyl-(thryptophan), CH₂NH₂ (2,3-diaminopropanoic acid), and (CH₂)₂NH₂ (2,4-diaminobutanoic acid),
R¹¹ and R¹² independently of each other represents hydrogen or a group selected from:
C₁-C₃-alkyl, C₂-C₃-haloalkyl, —C(═O)H, and —C(═O)—(C₁-C₃-alkyl),
R¹⁴ represents, independently of each other, a group selected from:
C₁-C₆-alkyl, C₃-C₆-cycloalkyl, phenyl, and benzyl,
wherein said group is optionally substituted with one, two or three substituents, identical or different, selected from the group of
hydroxy, halogen, and —NR¹¹R¹²,
R¹⁵ represents hydrogen or a group selected from:
cyano, and —C(═O)R¹⁶
R¹⁶ represents a group selected from:
C₁-C₆-alkyl, and C₁-C₆-haloalkyl,
R¹⁷ and R¹⁸ together with the nitrogen to which they are attached form
an azetidinyl group or a 5- to 7-membered heterocycloalkyl group,
said 5- to 7-membered heterocycloalkyl group optionally containing one additional heteroatom or heteroatom containing group selected from O, NH, S, S(═O), and S(═O)₂,
said azetidinyl group being optionally substituted with a substituent selected from:
C₁-C₄-alkyl, C₁-C₄-haloalkyl, C₁-C₄-alkoxy, C₁-C₄-haloalkoxy,
(C₁-C₃-alkoxy)-(C₁-C₄-alkyl)-, C₃-C₆-cycloalkyl, C₃-C₆-cycloalkyloxy, hydroxy,
halogen, cyano, —C(═O)H, (C₁-C₄-alkyl)-C(═O)—, —N(R¹¹)R¹²,
—(C₁-C₄-alkyl)-N(R¹¹)R¹², —C(═O)N(R¹¹)R¹², —(C₁-C₄-alkyl)-C(═O)N(R¹¹)R¹², and —C(═O)OH,
or being substituted with two halogen atoms,
said 5- to 7-membered heterocycloalkyl group being optionally substituted, one, two, three, four or five times, identically or differently, with a substituent selected from:
C₁-C₄-alkyl, C₁-C₄-haloalkyl, C₁-C₄-alkoxy, C₁-C₄-haloalkoxy,
(C₁-C₃-alkoxy)-(C₁-C₄-alkyl)-, C₃-C₆-cycloalkyl, C₃-C₆-cycloalkyloxy, hydroxy, a halogen atom, cyano, —C(═O)H, (C₁-C₄-alkyl)-C(═O)—,
—N(R¹¹)R¹², —(C₁-C₄-alkyl)-N(R¹¹)R¹², C(═O)N(R¹¹)R¹²,
—(C₁-C₄-alkyl)-C(═O)N(R¹¹)R¹², and —C(═O)OH, and
R¹⁹ represents hydrogen or a group selected from:
C₁-C₃-alkyl, C₁-C₃-haloalkyl, C₂-C₃-hydroxyalkyl, C₃-C₄-cycloalkyl,
(C₃-C₄-cycloalkyl)-(C₁-C₃-alkyl)-, and (C₁-C₃-alkoxy)-(C₂-C₃-alkyl)-,
or an N-oxide, a salt, a tautomer or a stereoisomer of said compound, or a salt of said N-oxide, tautomer or stereoisomer.

2. The compound according to claim 1,
wherein:
V, W, Y and Z independently of each other represent CH or CR³,
or,
V represents N, and W, Y and Z independently of each other represent CH or CR³,
or,
W represents N, and V, Y and Z independently of each other represent CH or CR³,
or,
V and Y represent N, and W and Z independently of each other represent CH or CR³,
R¹ and R² represent, independently of each other hydrogen, halogen, or a group selected from:
C₁-C₅-alkyl, C₃-C₆-cycloalkyl, C₁-C₅-haloalkyl, C₁-C₅-hydroxyalkyl,
(C₁-C₃-alkoxy)-(C₁-C₃-alkyl)-, —(C₁-C₅-alkyl)-S—R¹⁴, —(C₁-C₅-alkyl)-S(═O)—R¹⁴,
—(C₁-C₅-alkyl)-S(═O)₂—R¹⁴, —(C₁-C₅-alkyl)-S(═O)(═NR¹⁵)R¹⁴,
—(C₁-C₅-alkyl)-N(R¹¹)R¹², —(C₁-C₅-alkyl)-N(R¹⁷)R¹⁸, 5- or 6-membered heteroaryl,
a 5- to 7-membered oxygen containing heterocycloalkyl group,
(1,3-dioxolan-2-yl)-(C₁-C₅-alkyl)-, (1,3-dioxan-2-yl)-(C₁-C₅-alkyl)-, an azetidinyl group,
azetidinyl-(C₁-C₅-alkyl)-, a 5- to 7-membered heterocycloalkyl group, and
(5- to 7-membered heterocycloalkyl)-(C₁-C₅-alkyl)-,
in which 5- or 6-membered heteroaryl is connected to rest of the molecule via a carbon atom of the heteroaryl group,
in which 5- to 7-membered oxygen containing heterocycloalkyl is connected to rest of the molecule via a carbon atom of the 5- to 7-membered oxygen containing heterocycloalkyl group,
in which azetidinyl and 5- to 7-membered heterocycloalkyl are connected to rest of the molecule via a carbon atom of the azetidinyl ring or via a carbon atom of the 5- to 7-membered heterocycloalkyl ring,
wherein 5- or 6-membered heteroaryl is optionally substituted, one or two times, with a methyl group,
wherein azetidinyl is optionally substituted with a substituent selected from:
hydroxy, a halogen atom, cyano, C₁-C₄-alkyl, C₁-C₄-haloalkyl,
C₁-C₄-alkoxy, C₁-C₄-haloalkoxy, (C₁-C₃-alkoxy)-(C₁-C₄-alkyl)-,
C₃-C₄-cycloalkyl, C₃-C₄-cycloalkyloxy, —N(R¹¹)R¹², and
—N(H)C(═O)—(C₁-C₃-alkyl),
or being substituted with two halogen atoms,
wherein 5- to 7-membered heterocycloalkyl is optionally substituted, one, two, three, four or five times, identically or differently, with a substituent selected from:
hydroxy, halogen, cyano, C₁-C₄-alkyl, C₁-C₄-haloalkyl, C₁-C₄-alkoxy,
C₁-C₄-haloalkoxy, (C₁-C₃-alkoxy)-(C₁-C₄-alkyl)-, C₃-C₄-cycloalkyl,
(C₃-C₄-cycloalkyl)methyl-C₃-C₄-cycloalkyloxy, —N(R¹¹)R¹², —C(═O)OH and —N(H)C(═O)—(C₁-C₃-alkyl),
wherein at least one of R¹ and R² represents a group which connects via a carbon atom of said group to the rest of the molecule,
or,
R¹ and R² together represent a group, which is selected from: 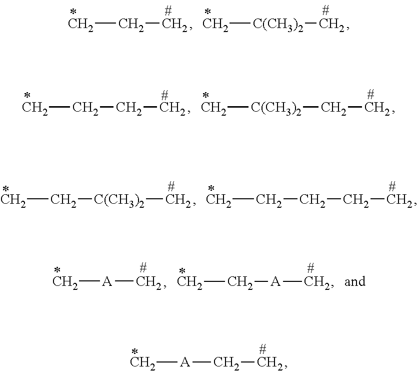
wherein A represents a group, which is selected from:
NH, N—(C₁-C₃-alkyl), N—(C₂-C₃-haloalkyl), N-benzyl, O, S, S(═O), S(═O)₂, and
S(═O)(═NR¹⁵)R¹⁴,
and,
wherein * indicates the point of attachment of R¹ to the rest of the molecule,
and # indicates the point of attachment of R² to the rest of the molecule,
R³ represents, independently of each other, halogen or a group selected from:
hydroxy, C₁-C₃-alkyl, C₃-C₄-cycloalkyl, C₁-C₃-haloalkyl, C₁-C₃-alkoxy,
C₁-C₃-haloalkoxy, —N(H)C(═O)H, —N(H)C(═O)—(C₁-C₃-alkyl),
—N(H)C(═O)—(C₁-C₃-alkyl)-(C₁-C₃-alkoxy), —N(H)C(═O)-phenyl,
—N(H)C(═O)—(C₃-C₄-cycloalkyl), —N(H)C(═O)—(C₁-C₃-hydroxyalkyl), —C(═O)OH,
—C(═O)NH₂, —S(═O)₂—(C₁-C₃-alkyl), —N(H)C(═O)—(C₁-C₃-alkyl)-(C₃-C₄-cycloalkyl), and —N(H)C(═O)N(H)R¹⁹,
said —N(H)C(═O)-phenyl being optionally substituted at the phenyl ring, one, two or three times, identically or differently, with a substituent selected from:
a halogen atom, hydroxy, cyano, C₁-C₄-alkyl, C₁-C₄-haloalkyl,
C₁-C₄-alkoxy, C₁-C₄-haloalkoxy, C₃-C₄-cycloalkyl, and
C₃-C₄-cycloalkyloxy,
said —N(H)C(═O)—(C₃-C₄-cycloalkyl) being optionally substituted at the C₃-C₄-cycloalkyl ring with a substituent selected from:
fluorine, chlorine, trifluoromethyl, and methoxy,
R⁴ and R⁵ represent, independently of each other, hydrogen or fluorine,
R⁶ represents a group selected from:
hydroxy, C₁-C₄-alkyl, C₁-C₄-haloalkyl, C₁-C₄-hydroxyalkyl,
(C₁-C₃-alkoxy)-(C₁-C₃-alkyl)-, C₃-C₄-cycloalkyl, (C₃-C₄-cycloalkyl)-(C₁-C₃-alkyl)-, C₁-C₄-alkoxy, C₁-C₄-haloalkoxy, (C₂-C₄-hydroxyalkyl)-O—,
(C₁-C₃-alkoxy)-(C₂-C₃-alkoxy)-, C₃-C₄-cycloalkyloxy,
(C₃-C₄-cycloalkyl)-(C₁-C₃-alkoxy)-, and R⁸,
wherein said hydroxyalkyl groups are optionally substituted with one, two or three halogen atoms selected from:
fluorine, and chlorine,
R⁷ represents hydrogen, or a 2-hydroxyethyl group,
or,
R² and R⁷ together represent a 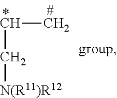
wherein * indicates the point of attachment of R² to the rest of the molecule,
and # indicates the point of attachment of R⁷ to the rest of the molecule,
R⁸ represents a group selected from:
—(C₂-C₄-alkyl)-OC(═O)—C(H)(R⁹)—N(H)C(═O)—C(H)(R¹⁰)—NH₂, and
—O—(C₂-C₄-alkyl)-OC(═O)—C(H)(R⁹)—N(H)C(═O)—C(H)(R¹⁰)—NH₂,
wherein said C₂-C₄-alkyl groups are optionally substituted with one, two or three halogen atoms selected from:
fluorine, and chlorine,
R⁹ and R¹⁰ independently of each other represent hydrogen (glycine) or a group selected from:
CH₃ (alanine), C(H)(CH₃)₂ (valine), (CH₂)₂CH₃ (norvaline), CH₂C(H)(CH₃)₂ (leucine),
C(H)(CH₃)CH₂CH₃ (isoleucine), (CH₂)₃CH₃ (norleucine), C(CH₃)₃ (2-tert-butylglycine),
benzyl (phenylalanine), 4-hydroxybenzyl (tyrosine),
(CH₂)₃NH₂ (ornithine), (CH₂)₄NH₂ (lysine), (CH₂)₂C(H)(OH)CH₂NH₂ (hydroxylysine),
CH₂OH (serine), (CH₂)₂OH (homoserine), C(H)(OH)CH₃ (threonine),
(CH₂)₃N(H)C(═NH)NH₂ (arginine), (CH₂)₃N(H)C(═O)NH₂ (citrulline), CH₂C(═O)NH₂ (asparagine), CH₂C(═O)OH (aspartic acid), (CH₂)₂C(═O)OH (glutamic acid),
(CH₂)₂C(═O)NH₂ (glutamine), CH₂SH (cysteine), (CH₂)₂SH (homocysteine), (CH₂)₂SCH₃ (methionine), CH₂SCH₃ (S-methylcysteine), (1H-imidazol-4-yl)methyl-(histidine),
(1H-indol-3-yl)methyl-(thryptophan), CH₂NH₂ (2,3-diaminopropanoic acid), and
(CH₂)₂NH₂ (2,4-diaminobutanoic acid),
R¹¹ and R¹² independently of each other represents hydrogen or a group selected from:
C₁-C₃-alkyl, C₂-C₃-haloalkyl, —C(═O)H, and —C(═O)—(C₁-C₃-alkyl),
R¹⁴ represents, independently of each other, a group selected from:
C₁-C₄-alkyl, C₃-C₄-cycloalkyl, phenyl, and benzyl,
wherein said group is optionally substituted with one, two or three substituents, identical or different, selected from the group of
hydroxy, halogen, and —NR¹¹R¹²,
R¹⁵ represents hydrogen or a group selected from:
cyano, and —C(═O)R¹⁶
R¹⁶ represents a group selected from:
C₁-C₃-alkyl, and C₁-C₃-haloalkyl,
R¹⁷ and R¹⁸ together with the nitrogen to which they are attached form:
an azetidinyl group or a 5- to 7-membered heterocycloalkyl group,
said 5- to 7-membered heterocycloalkyl group optionally containing one additional heteroatom or heteroatom containing group selected from O, NH, S, S(═O), and S(═O)₂,
said azetidinyl group being optionally substituted with a substituent selected from:
C₁-C₄-alkyl, C₁-C₄-haloalkyl, C₁-C₄-alkoxy, C₁-C₄-haloalkoxy,
(C₁-C₃-alkoxy)-(C₁-C₄-alkyl)-, C₃-C₄-cycloalkyl, C₃-C₄-cycloalkyloxy, hydroxy,
halogen, cyano, —C(═O)H, (C₁-C₄-alkyl)-C(═O)—, —N(R¹¹)R¹²,
—(C₁-C₄-alkyl)-N(R¹¹)R¹², —C(═O)N(R¹¹)R¹²,
—(C₁-C₄-alkyl)-C(═O)N(R¹¹)R¹², and —C(═O)OH,
or being substituted with two halogen atoms,
said 5- to 7-membered heterocycloalkyl group being optionally substituted, one, two, three, four or five times, identically or differently, with a substituent selected from:
C₁-C₄-alkyl, C₁-C₄-haloalkyl, C₁-C₄-alkoxy, C₁-C₄-haloalkoxy,
(C₁-C₃-alkoxy)-(C₁-C₄-alkyl)-, C₃-C₄-cycloalkyl, C₃-C₄-cycloalkyloxy, hydroxy, a
halogen atom, cyano, —C(═O)H, (C₁-C₄-alkyl)-C(═O)—,
—N(R¹¹)R¹², —(C₁-C₄-alkyl)-N(R¹¹)R¹², —C(═O)N(R¹¹)R¹²,
—(C₁-C₄-alkyl)-C(═O)N(R¹¹)R¹², and —C(═O)OH, and
R¹⁹ represents hydrogen or a group selected from:
C₁-C₃-alkyl, C₁-C₃-haloalkyl, C₂-C₃-hydroxyalkyl, C₃-C₄-cycloalkyl,
(C₃-C₄-cycloalkyl)-(C₁-C₃-alkyl)-, and (C₁-C₃-alkoxy)-(C₂-C₃-alkyl)-,
or an N-oxide, a salt, a tautomer or a stereoisomer of said compound, or a salt of said N-oxide, tautomer or stereoisomer.

3. The compound according to claim 1,
wherein,
V, W, Y and Z independently of each other represent CH or CR³,
or,
V represents N, and W, Y and Z independently of each other represent CH or CR³,
or,
W represents N, and V, Y and Z independently of each other represent CH or CR³,
or,
V and Y represent N, and W and Z independently of each other represent CH or CR³,
R¹ represents hydrogen, halogen, or a group selected from:
C₁-C₅-alkyl, C₁-C₅-haloalkyl, C₁-C₅-hydroxyalkyl, (C₁-C₃-alkoxy)-(C₁-C₃-alkyl)-,
—(C₁-C₅-alkyl)-S—R¹⁴, —(C₁-C₆-alkyl)-S(═O)—R¹⁴, —(C₁-C₅-alkyl)-S(═O)₂—R¹⁴,
—(C₁-C₅-alkyl)-S(═O)(═NR¹⁵)R¹⁴, —(C₁-C₅-alkyl)-N(R¹¹)R¹²,
—(C₁-C₅-alkyl)-N(R¹⁷)R¹⁸, 5-membered heteroaryl, and a 5- or 6-membered oxygen containing heterocycloalkyl group,
in which 5-membered heteroaryl is connected to rest of the molecule via a carbon atom of the heteroaryl group,
wherein 5-membered heteroaryl is optionally substituted, one or two times, with a methyl group,
R² represents hydrogen, halogen, or a group selected from:
C₁-C₅-alkyl, C₃-C₆-cycloalkyl, C₁-C₅-haloalkyl, C₁-C₅-hydroxyalkyl,
(C₁-C₃-alkoxy)-(C₁-C₃-alkyl)-, —(C₁-C₅-alkyl)-S—R¹⁴, —(C₁-C₅-alkyl)-S(═O)—R¹⁴,
—(C₁-C₅-alkyl)-S(═O)₂—R¹⁴, —(C₁-C₅-alkyl)-S(═O)(═NR¹⁵)R¹⁴,
—(C₁-C₅-alkyl)-N(R¹¹)R¹², —(C₁-C₆-alkyl)-N(R¹⁷)R¹⁸,
(1,3-dioxolan-2-yl)-(C₁-C₅-alkyl)-, (1,3-dioxan-2-yl)-(C₁-C₅-alkyl)-, an azetidinyl group,
azetidinyl-(C₁-C₅-alkyl)-, a 5- to 7-membered heterocycloalkyl group, and
(5- to 7-membered heterocycloalkyl)-(C₁-C₅-alkyl)-,
in which azetidinyl and 5- to 7-membered heterocycloalkyl are connected to rest of the molecule via a carbon atom of the azetidinyl ring or via a carbon atom of the 5- to 7-membered heterocycloalkyl ring,
wherein azetidinyl is optionally substituted with a substituent selected from:
a halogen atom, C₁-C₄-alkyl, and C₁-C₄-haloalkyl,
or being substituted with two halogen atoms,
wherein 5- to 7-membered heterocycloalkyl is optionally substituted, one, two, three, four or five times, identically or differently, with a substituent selected from:
halogen, C₁-C₄-alkyl, C₁-C₄-haloalkyl and cyclopropylmethyl-,
wherein at least one of R¹ and R² represents a group which connects via a carbon atom of said group to the rest of the molecule,
or,
R¹ and R² together represent a group, which is selected from: 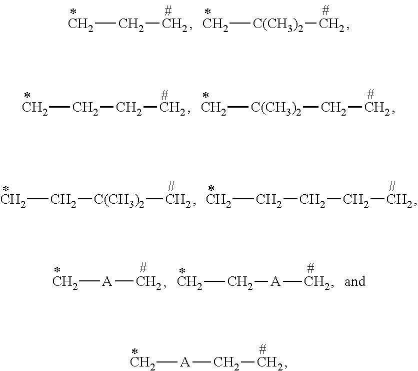
wherein A represents a group, which is selected from:
NH, N—(C₁-C₃-alkyl), N—(C₂-C₃-haloalkyl), N-benzyl, O, S, S(═O), and S(═O)₂,
and,
wherein * indicates the point of attachment of R¹ to the rest of the molecule,
and # indicates the point of attachment of R² to the rest of the molecule,
R³ represents, independently of each other, halogen or a group selected from:
hydroxy, C₁-C₃-alkyl, C₃-C₄-cycloalkyl, C₁-C₃-haloalkyl, C₁-C₃-alkoxy,
C₁-C₃-haloalkoxy, —N(H)C(═O)H, —N(H)C(═O)—(C₁-C₃-alkyl),
—N(H)C(═O)—(C₃-C₄-cycloalkyl), —N(H)C(═O)—(C₁-C₃-hydroxyalkyl), —C(═O)OH,
—C(═O)NH₂, and —S(═O)₂—(C₁-C₃-alkyl),
said —N(H)C(═O)—(C₃-C₄-cycloalkyl) being optionally substituted at the C₃-C₄-cycloalkyl ring with a substituent selected from:
fluorine, chlorine, trifluoromethyl, and methoxy,
R⁴ and R⁵ represent, independently of each other, hydrogen or fluorine,
R⁶ represents a group selected from:
hydroxy, C₁-C₄-alkoxy, C₁-C₄-haloalkoxy, (C₂-C₄-hydroxyalkyl)-O—,
(C₁-C₃-alkoxy)-(C₂-C₃-alkoxy)-, and R⁸,
R⁷ represents hydrogen, or a 2-hydroxyethyl group,
or,
R² and R⁷ together represent a 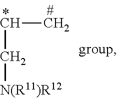
wherein * indicates the point of attachment of R² to the rest of the molecule,
and # indicates the point of attachment of R⁷ to the rest of the molecule,
R⁸ represents a group selected from:
—(C₂-C₄-alkyl)-OC(═O)—C(H)(R⁹)—N(H)C(═O)—C(H)(R¹⁰)—NH₂, and
—O—(C₂-C₄-alkyl)-OC(═O)—C(H)(R⁹)—N(H)C(═O)—C(H)(R¹⁰)—NH₂,
R⁹ and R¹⁰ independently of each other represent a group selected from:
CH₃ (alanine), C(H)(CH₃)₂ (valine), (CH₂)₂CH₃ (norvaline), (CH₂)₃NH₂ (ornithine),
(CH₂)₄NH₂ (lysine), and (CH₂)₃N(H)C(═NH)NH₂ (arginine),
R¹¹ and R¹² independently of each other represents hydrogen or a group selected from:
C₁-C₃-alkyl, C₂-C₃-haloalkyl, —C(═O)H, and —C(═O)—(C₁-C₃-alkyl),
R¹⁴ represents, independently of each other, a group selected from:
C₁-C₄-alkyl, C₃-C₄-cycloalkyl,
R¹⁵ represents hydrogen or a group selected from:
cyano, and —C(═O)R¹⁶
R¹⁶ represents a group selected from:
C₁-C₃-alkyl, and C₁-C₃-haloalkyl, and
R¹⁷ and R¹⁸ together with the nitrogen to which they are attached form:
an azetidinyl group or a 5- to 7-membered heterocycloalkyl group,
said 5- to 7-membered heterocycloalkyl group optionally containing one additional heteroatom or heteroatom containing group selected from O, NH, S, S(═O), and S(═O)₂,
said azetidinyl group being optionally substituted with a substituent selected from:
C₁-C₄-alkyl, C₁-C₄-haloalkyl, halogen,
or being substituted with two halogen atoms,
said 5- to 7-membered heterocycloalkyl group being optionally substituted, one, two, three, four or five times, identically or differently, with a substituent selected from:
C₁-C₄-alkyl, C₁-C₄-haloalkyl, and a halogen atom,
or an N-oxide, a salt, a tautomer or a stereoisomer of said compound, or a salt of said N-oxide, tautomer or stereoisomer.

4. The compound according to claim 1, wherein,
V, W, Y and Z independently of each other represent CH or CR³,
or,
V represents N, and W, Y and Z independently of each other represent CH or CR³,
or,
W represents N, and V, Y and Z independently of each other represent CH or CR³,
or,
V and Y represent N, and W and Z independently of each other represent CH or CR³,
R¹ represents hydrogen, or a group selected from:
C₁-C₅-alkyl, C₁-C₅-haloalkyl, C₁-C₅-hydroxyalkyl, (C₁-C₃-alkoxy)-(C₁-C₃-alkyl)-,
—(C₁-C₅-alkyl)-S—R¹⁴, —(C₁-C₅-alkyl)-S(═O)₂—R¹⁴, —(C₁-C₅-alkyl)-N(R¹⁷)R¹⁸,
5-membered heteroaryl, and a 5- or 6-membered oxygen containing heterocycloalkyl group,
in which 5-membered heteroaryl is connected to rest of the molecule via a carbon atom of the heteroaryl group,
wherein 5-membered heteroaryl is optionally substituted, one or two times, with a methyl group,
R² represents hydrogen, halogen, or a group selected from:
C₁-C₅-alkyl, cyclopropyl, C₁-C₅-hydroxyalkyl, (C₁-C₃-alkoxy)-(C₁-C₃-alkyl)-,
—(C₁-C₅-alkyl)-S—R¹⁴, —(C₁-C₅-alkyl)-S(═O)—R¹⁴, —(C₁-C₅-alkyl)-S(═O)₂—R¹⁴,
—(C₁-C₅-alkyl)-S(═O)(═NR¹⁵)R¹⁴, —(C₁-C₅-alkyl)-N(R¹¹)R¹²,
—(C₁-C₅-alkyl)-N(R¹⁷)R¹⁸, (1,3-dioxolan-2-yl)-(C₁-C₅-alkyl)-, an azetidinyl group, azetidinyl-(C₁-C₅-alkyl)-, and a 5- to 7-membered heterocycloalkyl group,
in which azetidinyl and 5- to 7-membered heterocycloalkyl are connected to rest of the molecule via a carbon atom of the azetidinyl ring or via a carbon atom of the 5- to 7-membered heterocycloalkyl ring,
wherein azetidinyl is optionally substituted with a substituent selected from:
C₁-C₄-haloalkyl,
wherein 5- to 7-membered heterocycloalkyl is optionally substituted with a substituent selected from:
C₁-C₄-haloalkyl and cyclopropylmethyl-,
wherein at least one of R¹ and R² represents a group which connects via a carbon atom of said group to the rest of the molecule,
or,
R¹ and R² together represent a group, which is selected from: 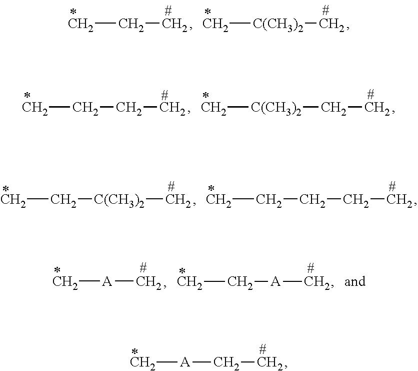
wherein A represents a group, which is selected from:
NH, N—(C₁-C₃-alkyl), N-(2,2,2-trifluoroethyl), N-benzyl, O, S, and S(═O)₂,
and,
wherein * indicates the point of attachment of R¹ to the rest of the molecule,
and # indicates the point of attachment of R² to the rest of the molecule,
R³ represents, independently of each other, halogen or a group selected from:
hydroxy, C₁-C₃-alkyl, C₃-C₄-cycloalkyl, C₁-C₃-haloalkyl, C₁-C₃-alkoxy,
C₁-C₃-haloalkoxy, —N(H)C(═O)—(C₁-C₃-alkyl), —N(H)C(═O)—(C₃-C₄-cycloalkyl),
—C(═O)OH, —C(═O)NH₂, and —S(═O)₂—(C₁-C₃-alkyl),
R⁴ and R⁵ represent, independently of each other, hydrogen or fluorine,
R⁶ represents a group selected from:
hydroxy, C₁-C₄-alkoxy, (C₂-C₄-hydroxyalkyl)-O—, and
(C₁-C₃-alkoxy)-(C₂-C₃-alkoxy)-,
R⁷ represents hydrogen, or a 2-hydroxyethyl group,
or,
R² and R⁷ together represent a 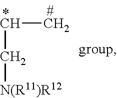
wherein * indicates the point of attachment of R² to the rest of the molecule,
and # indicates the point of attachment of R⁷ to the rest of the molecule,
R¹¹ and R¹² independently of each other represents hydrogen or a group selected from:
C₂-C₃-haloalkyl, and —C(═O)H,
R¹⁴ represents, independently of each other, a group selected from:
C₁-C₄-alkyl,
R¹⁵ represents hydrogen or a group selected from:
—C(═O)R¹⁶
R¹⁶ represents a group selected from:
C₁-C₃-haloalkyl, and
R¹⁷ and R¹⁸ together with the nitrogen to which they are attached form:
an azetidinyl group or a 5- to 7-membered heterocycloalkyl group,
said 5- to 7-membered heterocycloalkyl group optionally containing one additional heteroatom selected from O,
said azetidinyl group being substituted with two halogen atoms,
said 5- to 7-membered heterocycloalkyl group being optionally substituted, two times, identically or differently, with a substituent selected from:
halogen atom,
or an N-oxide, a salt, a tautomer or a stereoisomer of said compound, or a salt of said N-oxide, tautomer or stereoisomer.

5. The compound according to claim 1, wherein
V, W, Y and Z independently of each other represent CH or CR³,
or,
V represents N, and W, Y and Z independently of each other represent CH or CR³,
or,
W represents N, and V, Y and Z independently of each other represent CH or CR³,
or,
V and Y represent N, and W and Z represent CH,
R¹ represents hydrogen, or a group selected from:
C₁-C₃-alkyl, trifluoromethyl, C₁-C₂-hydroxyalkyl, methoxymethyl-, —(CH₂)—S—R¹⁴,
—(CH₂)—S(═O)₂—R¹⁴, —(CH₂)—N(R¹⁷)R¹⁸, 5-membered heteroaryl, and a 5- or 6-membered oxygen containing heterocycloalkyl group,
in which 5-membered heteroaryl is connected to rest of the molecule via a carbon atom of the heteroaryl group,
which 5-membered heteroaryl group is selected from:
1,3-thiazolyl and pyrazolyl, which groups are optionally substituted, one or two times with a methyl group,
which 5- or 6-membered oxygen containing heterocycloalkyl group is selected from:
tetrahydrofuranyl and tetrahydro-2H-pyranyl,
R² represents hydrogen, fluorine, chlorine, or a group selected from:
C₁-C₂-alkyl, cyclopropyl, —(CH₂)₃OH, methoxy-(C₂-C₃-alkyl)-, —(C₁-C₃-alkyl)-S—R¹⁴,
—(C₁-C₃-alkyl)-S(═O)—R¹⁴, —(C₁-C₃-alkyl)-S(═O)₂—R¹⁴, —(CH₂)₂—S(═O)(═NR¹⁵)R¹⁴,
—(CH₂)₃—S(═O)(═NR¹⁵)R¹⁴, —(C₁-C₄-alkyl)-N(R¹¹)R¹², —(C₁-C₄-alkyl)-N(R¹⁷)R¹⁸,
(1,3-dioxolan-2-yl)-(CH₂)—, an azetidinyl group, azetidinyl-(CH₂)—, and a piperidinyl group,
in which azetidinyl and piperidinyl are connected to rest of the molecule via a carbon atom of the azetidinyl ring or via a carbon atom of the piperidinyl ring,
wherein azetidinyl is optionally substituted with a 2,2,2-trifluoroethyl group,
wherein piperidinyl is optionally substituted with a substituent selected from:
2,2,2-trifluoroethyl, 2,2-difluoroethyl, 3,3,3-trifluoropropyl and cyclopropylmethyl-,
wherein at least one of R¹ and R² represents a group which connects via a carbon atom of said group to the rest of the molecule,
or,
R¹ and R² together represent a group, which is selected from: 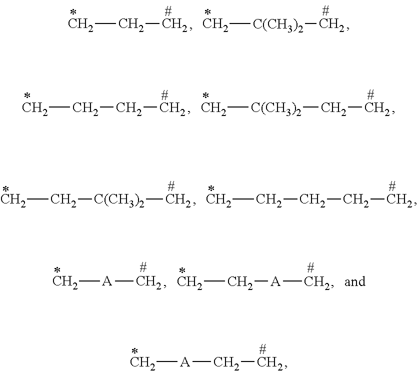
wherein A represents a group, which is selected from:
NH, N-ethyl, N-(2,2,2-trifluoroethyl), N-benzyl, O, S, and S(═O)₂,
and,
wherein * indicates the point of attachment of R¹ to the rest of the molecule,
and # indicates the point of attachment of R² to the rest of the molecule,
R³ represents fluorine, chlorine or a group selected from:
hydroxy, methyl, cyclopropyl, difluoromethyl, trifluoromethyl, methoxy,
trifluoromethoxy, —N(H)C(═O)—CH₃, —N(H)C(═O)-cyclopropyl, —C(═O)OH,
—C(═O)NH₂, and —S(═O)₂—CH₃,
R⁴ and R⁵ represent, independently of each other, hydrogen or fluorine,
R⁶ represents a group selected from:
hydroxy, C₁-C₂-alkoxy, (2-hydroxyethyl)-O—, and 2-methoxyethoxy-,
R⁷ represents hydrogen, or a 2-hydroxyethyl group,
or,
R² and R⁷ together represent a 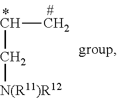
wherein * indicates the point of attachment of R² to the rest of the molecule,
and # indicates the point of attachment of R⁷ to the rest of the molecule,
R¹¹ and R¹² independently of each other represents hydrogen or a group selected from:
2,2,2-trifluoroethyl, and C(═O)H,
R¹⁴ represents a methyl group,
R¹⁵ represents hydrogen or a group selected from:
—C(═O)R¹⁶
R¹⁶ represents a trifluoromethyl group, and
R¹⁷ and R¹⁸ together with the nitrogen to which they are attached form:
an azetidinyl group or a 6-membered heterocycloalkyl group,
said 6-membered heterocycloalkyl group optionally containing one additional heteroatom selected from O,
said azetidinyl group being substituted with two fluorine atoms,
said 6-membered heterocycloalkyl group being optionally substituted with two fluorine atoms,
or an N-oxide, a salt, a tautomer or a stereoisomer of said compound, or a salt of said N-oxide, tautomer or stereoisomer.

6. The compound according to claim 1,
wherein
V, W, Y and Z independently of each other represent CH or CR³,
or,
V represents N, and W, Y and Z independently of each other represent CH or CR³,
or,
W represents N, and V, Y and Z independently of each other represent CH or CR³,
or,
V and Y represent N, and W and Z represent CH,
R¹ represents hydrogen, or a group selected from:
C₁-C₃-alkyl, C₁-C₂-hydroxyalkyl, methoxymethyl-, —(CH₂)—S—R¹⁴,
—(CH₂)—S(═O)₂—R¹⁴, and —(CH₂)—N(R¹⁷)R¹⁸,
R² represents hydrogen, fluorine, chlorine, or a group selected from:
C₁-C₂-alkyl, —(CH₂)₃OH, methoxy-(C₂-C₃-alkyl)-, —(C₁-C₃-alkyl)-S—R¹⁴,
—(C₁-C₃-alkyl)-S(═O)—R¹⁴, —(C₁-C₃-alkyl)-S(═O)₂—R¹⁴,
—(CH₂)₃—S(═O)(═NR¹⁵)R¹⁴, —(C₁-C₄-alkyl)-N(R¹¹)R¹²,
—(C₁-C₄-alkyl)-N(R¹⁷)R¹⁸, (1,3-dioxolan-2-yl)-(CH₂)—, an azetidinyl group, azetidinyl-(CH₂)—, and a piperidinyl group,
in which azetidinyl and piperidinyl are connected to rest of the molecule via a carbon atom of the azetidinyl ring or via a carbon atom of the piperidinyl ring,
wherein azetidinyl is optionally substituted with a 2,2,2-trifluoroethyl group,
wherein piperidinyl is optionally substituted with a 2,2,2-trifluoroethyl group,
wherein at least one of R¹ and R² represents a group which connects via a carbon atom of said group to the rest of the molecule,
or,
R¹ and R² together represent a group, which is selected from: 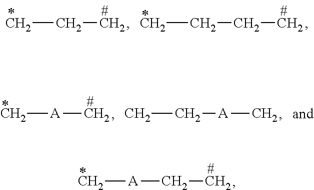
wherein A represents a group, which is selected from:
NH, N-ethyl, N-benzyl, O, S, and S(═O)₂,
and,
wherein * indicates the point of attachment of R¹ to the rest of the molecule,
and # indicates the point of attachment of R² to the rest of the molecule,
R³ represents fluorine, chlorine or a group selected from:
hydroxy, methyl, cyclopropyl, difluoromethyl, trifluoromethyl, methoxy,
—N(H)C(═O)—CH₃, —N(H)C(═O)-cyclopropyl, —C(═O)OH, —C(═O)NH₂, and
—S(═O)₂—CH₃,
R⁴ and R⁵ represent, independently of each other, hydrogen or fluorine,
R⁶ represents a group selected from:
hydroxy, C₁-C₂-alkoxy, (2-hydroxyethyl)-O—, and methoxy-ethoxy-,
R⁷ represents hydrogen, or a 2-hydroxyethyl group,
or,
R² and R⁷ together represent a 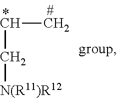
wherein * indicates the point of attachment of R² to the rest of the molecule,
and # indicates the point of attachment of R⁷ to the rest of the molecule,
R¹¹ and R¹² independently of each other represents hydrogen or a group selected from:
2,2,2-trifluoroethyl, and C(═O)H,
R¹⁴ represents a methyl group,
R¹⁵ represents hydrogen or a group selected from:
—C(═O)R¹⁶
R¹⁶ represents a trifluoromethyl group, and
R¹⁷ and R¹⁸ together with the nitrogen to which they are attached form:
an azetidinyl group or a 6-membered heterocycloalkyl group,
said 6-membered heterocycloalkyl group optionally containing one additional heteroatom selected from O,
said azetidinyl group being substituted with two fluorine atoms,
said 6-membered heterocycloalkyl group being optionally substituted with two fluorine atoms,
or an N-oxide, a salt, a tautomer or a stereoisomer of said compound, or a salt of said N-oxide, tautomer or stereoisomer.

7. The compound of formula (I) according to claim 1, which is selected from the group consisting of:
2-[1-(4-ethoxy-2,6-difluorobenzyl)-1H-indazol-3-yl]-5-{2-[(R)-methylsulfinyl]ethyl}-N-(pyridin-4-yl)pyrimidin-4-amine;
2-[1-(4-ethoxy-2,6-difluorobenzyl)-1H-indazol-3-yl]-5-{3-[(R)-methylsulfinyl]-propyl}-N-(pyridin-4-yl)pyrimidin-4-amine;
2-[1-(4-ethoxy-2,6-difluorobenzyl)-1H-indazol-3-yl]-5-[2-(methylsulfanyl)ethyl]-N-(pyridin-4-yl)pyrimidin-4-amine;
2-[1-(4-ethoxy-2,6-difluorobenzyl)-1H-indazol-3-yl]-5-[3-(methylsulfanyl)propyl]-N-(pyridin-4-yl)pyrimidin-4-amine;
2-[1-(4-ethoxy-2,6-difluorobenzyl)-1H-indazol-3-yl]-5-[(methylsulfanyl)methyl]-N-(pyridin-4-yl)pyrimidin-4-amine;
2-[1-(4-ethoxy-2,6-difluorobenzyl)-1H-indazol-3-yl]-5-methyl-N-(pyridin-4-yl)-pyrimidin-4-amine;
N-({2-[1-(4-ethoxy-2,6-difluorobenzyl)-1H-indazol-3-yl]-4-(pyridin-4-ylamino)-pyrimidin-5-yl}methyl)formamide;
2-[1-(4-ethoxy-2,6-difluorobenzyl)-1H-indazol-3-yl]-5-(2-methoxyethyl)-N-(pyridin-4-yl)pyrimidin-4-amine;
4-({2-[1-(4-ethoxy-2,6-difluorobenzyl)-1H-indazol-3-yl]-5-(3-methoxypropyl)-pyrimidin-4-yl}amino)nicotinic acid;
2-[1-(4-ethoxy-2,6-difluorobenzyl)-1H-indazol-3-yl]-5-(3-methoxypropyl)-N-(pyridin-4-yl)pyrimidin-4-amine;
2-[1-(4-ethoxy-2,6-difluorobenzyl)-1H-indazol-3-yl]-5-[4-(morpholin-4-yl)butyl]-N-(pyridin-4-yl)pyrimidin-4-amine;
2-[1-(4-ethoxy-2,6-difluorobenzyl)-1H-indazol-3-yl]-5-[3-(morpholin-4-yl)propyl]-N-(pyridin-4-yl)pyrimidin-4-amine;
2-[1-(4-ethoxy-2,6-difluorobenzyl)-1H-indazol-3-yl]-5-[3-(morpholin-4-yl)propyl]-N-[3-(trifluoromethyl)pyridin-4-yl]pyrimidin-4-amine;
5-(1,3-dioxolan-2-ylmethyl)-2-[1-(4-ethoxy-2,6-difluorobenzyl)-1H-indazol-3-yl]-N-(pyridin-4-yl)pyrimidin-4-amine;
5-[2-(3,3-difluoropiperidin-1-yl)ethyl]-2-[1-(4-ethoxy-2,6-difluorobenzyl)-1H-indazol-3-yl]-N-(pyridin-4-yl)pyrimidin-4-amine;
5-[4-(3,3-difluoropiperidin-1-yl)butyl]-2-[1-(4-ethoxy-2,6-difluorobenzyl)-1H-indazol-3-yl]-N-(pyridin-4-yl)pyrimidin-4-amine;
5-[3-(4,4-difluoropiperidin-1-yl)propyl]-2-[1-(4-ethoxy-2,6-difluorobenzyl)-1H-indazol-3-yl]-N-(pyridin-4-yl)pyrimidin-4-amine;
5-[(3,3-difluoroazetidin-1-yl)methyl]-2-[1-(4-ethoxy-2,6-difluorobenzyl)-1H-indazol-3-yl]-N-(pyridin-4-yl)pyrimidin-4-amine;
5-[4-(3,3-difluoroazetidin-1-yl)butyl]-2-[1-(4-ethoxy-2,6-difluorobenzyl)-1H-indazol-3-yl]-N-(pyridin-4-yl)pyrimidin-4-amine;
2-[1-(4-ethoxy-2,6-difluorobenzyl)-1H-indazol-3-yl]-5-[2-(methylsulfonyl)ethyl]-N-(31eparate-4-yl)pyrimidin-4-amine;
2-[1-(4-ethoxy-2,6-difluorobenzyl)-1H-indazol-3-yl]-5-[3-(methylsulfonyl)propyl]-N-(pyridin-4-yl)pyrimidin-4-amine;
2-[1-(4-ethoxy-2,6-difluorobenzyl)-1H-indazol-3-yl]-5-[(methylsulfonyl)methyl]-N-(pyridin-4-yl)pyrimidin-4-amine;
2-[1-(4-ethoxy-2,6-difluorobenzyl)-1H-indazol-3-yl]-6-[(methylsulfonyl)methyl]-N-(pyridin-4-yl)pyrimidin-4-amine;
2-{3,5-difluoro-4-[(3-{5-[2-(methylsulfonyl)ethyl]-4-(31eparate-4-ylamino)pyrimidin-2-yl}-1H-indazol-1-yl)methyl]phenoxy}ethanol;
2-{3,5-difluoro-4-[(3-{5-[3-(methylsulfonyl)propyl]-4-(pyridin-4-ylamino)pyrimidin-2-yl}-1H-indazol-1-yl)methyl]phenoxy}ethanol;
2-[3,5-difluoro-4-({3-[5-{3-[(R)-methylsulfinyl]propyl}-4-(pyridin-4-ylamino)-pyrimidin-2-yl]-1H-indazol-1-yl}methyl)phenoxy]ethanol;
2-[(2-{1-[2,6-difluoro-4-(2-hydroxyethoxy)benzyl]-1H-indazol-3-yl}-5-[(methyl-sulfonyl)methyl]pyrimidin-4-yl)(pyridin-4-yl)amino]ethanol;
2-{3,5-difluoro-4-[(3-{4-[(3-methoxypyridin-4-yl)amino]-6,7-dihydro-5H-cyclopenta[d]pyrimidin-2-yl}-1H-indazol-1-yl)methyl]phenoxy}ethanol;
N-{4-[(2-{1-[2,6-difluoro-4-(2-hydroxyethoxy)benzyl]-1H-indazol-3-yl}-6,7-dihydro-5H-cyclopenta[d]pyrimidin-4-yl)amino]pyridin-2-yl}acetamide;
2-{4-[(3-{4-[(2-aminopyridin-4-yl)amino]-6,7-dihydro-5H-cyclopenta[d]pyrimidin-2-yl}-1H-indazol-1-yl)methyl]-3,5-difluorophenoxy}ethanol;
2-{3,5-difluoro-4-[(3-{4-[(3-methylpyridin-4-yl)amino]-6,7-dihydro-5H-cyclopenta[d]pyrimidin-2-yl}-1H-indazol-1-yl)methyl]phenoxy}ethanol;
N-[4-({2-[1-(4-ethoxy-2,6-difluorobenzyl)-1H-indazol-3-yl]-6-[(1S)-1-hydroxyethyl]pyrimidin-4-yl}amino)pyridin-2-yl]acetamide;
2-[3,5-difluoro-4-({3-[4-(pyridazin-4-ylamino)-6,7-dihydro-5H-cyclopenta[d]-pyrimidin-2-yl]-1H-indazol-1-yl}methyl)phenoxy]ethanol, salt with hydrochloric acid;
2-[3,5-difluoro-4-({3-[4-(pyridazin-4-ylamino)-6,7-dihydro-5H-cyclopenta[d]-pyrimidin-2-yl]-1H-indazol-1-yl}methyl)phenoxy]ethanol;
2-{3,5-difluoro-4-[(3-{4-[(6-methylpyrimidin-4-yl)amino]-6,7-dihydro-5H-cyclopenta[d]pyrimidin-2-yl}-1H-indazol-1-yl)methyl]phenoxy}ethanol;
2-{3,5-difluoro-4-[(3-{4-[(2-methylpyridin-4-yl)amino]-6,7-dihydro-5H-cyclopenta[d]pyrimidin-2-yl}-1H-indazol-1-yl)methyl]phenoxy}ethanol;
3,5-difluoro-4-[(3-{5-[2-(methylsulfonyl)ethyl]-4-(pyridin-4-ylamino)pyrimidin-2-yl}-1H-indazol-1-yl)methyl]phenol;
3,5-difluoro-4-[(3-{5-[3-(methylsulfonyl)propyl]-4-(pyridin-4-ylamino)pyrimidin-2-yl}-1H-indazol-1-yl)methyl]phenol;
3,5-difluoro-4-({3-[5-{3-[(R)-methylsulfinyl]propyl}-4-(pyridin-4-ylamino)pyrimidin-2-yl]-1H-indazol-1-yl}methyl)phenol;
{2-[1-(4-ethoxy-2,6-difluorobenzyl)-1H-indazol-3-yl]-6-[(3-methylpyridin-4-yl)-amino]pyrimidin-4-yl}methanol;
3,5-difluoro-4-[(3-{4-[(3-methylpyridin-4-yl)amino]-6,7-dihydro-5H-cyclopenta[d]-pyrimidin-2-yl}-1H-indazol-1-yl)methyl]phenol;
3,5-difluoro-4-[(3-{4-[(3-methoxypyridin-4-yl)amino]-6,7-dihydro-5H-cyclopenta[d]pyrimidin-2-yl}-1H-indazol-1-yl)methyl]phenol;
N-[4-({2-[1-(2,6-difluoro-4-hydroxybenzyl)-1H-indazol-3-yl]-6,7-dihydro-5H-cyclopenta[d]pyrimidin-4-yl}amino)pyridin-2-yl]acetamide;
3,5-difluoro-4-[(3-{4-[(2-methylpyridin-4-yl)amino]-6,7-dihydro-5H-cyclopenta[d]-pyrimidin-2-yl}-1H-indazol-1-yl)methyl]phenol;
3,5-difluoro-4-({3-[4-(pyridazin-4-ylamino)-6,7-dihydro-5H-cyclopenta[d]pyrimidin-2-yl]-1H-indazol-1-yl}methyl)phenol;
3,5-difluoro-4-[(3-{4-[(6-methylpyrimidin-4-yl)amino]-6,7-dihydro-5H-cyclopenta[d]pyrimidin-2-yl}-1H-indazol-1-yl)methyl]phenol;
3,5-difluoro-4-({3-[4-(pyrimidin-4-ylamino)-6,7-dihydro-5H-cyclopenta[d]pyrimidin-2-yl]-1H-indazol-1-yl}methyl)phenol;
3,5-difluoro-4-[(3-{4-[(2-methylpyrimidin-4-yl)amino]-6,7-dihydro-5H-cyclopenta[d]pyrimidin-2-yl}-1H-indazol-1-yl)methyl]phenol;
2-[1-(4-ethoxy-2,6-difluorobenzyl)-1H-indazol-3-yl]-5-[2-(S-methylsulfonimidoyl)ethyl]-N-(pyridin-4-yl)pyrimidin-4-amine;
N-[(2-{2-[1-(4-ethoxy-2,6-difluorobenzyl)-1H-indazol-3-yl]-4-(pyridin-4-ylamino)-pyrimidin-5-yl}ethyl)(methyl)oxido-λ6-sulfanylidene]-2,2,2-trifluoroacetamide;
5-(azetidin-3-yl)-2-[1-(4-ethoxy-2,6-difluorobenzyl)-1H-indazol-3-yl]-N-(pyridin-4-yl)pyrimidin-4-amine, salt with hydrochloric acid;
5-(azetidin-3-yl)-2-[1-(4-ethoxy-2,6-difluorobenzyl)-1H-indazol-3-yl]-N-(pyridin-4-yl)pyrimidin-4-amine;
5-(2-aminoethyl)-2-[1-(4-ethoxy-2,6-difluorobenzyl)-1H-indazol-3-yl]-N-(pyridin-4-yl)pyrimidin-4-amine, salt with hydrochloric acid;
5-(2-aminoethyl)-2-[1-(4-ethoxy-2,6-difluorobenzyl)-1H-indazol-3-yl]-N-(pyridin-4-yl)pyrimidin-4-amine;
2-[1-(4-ethoxy-2,6-difluorobenzyl)-1H-indazol-3-yl]-5-(piperidin-4-yl)-N-(pyridin-4-yl)pyrimidin-4-amine, salt with hydrochloric acid;
2-[1-(4-ethoxy-2,6-difluorobenzyl)-1H-indazol-3-yl]-5-(piperidin-4-yl)-N-(pyridin-4-yl)pyrimidin-4-amine;
5-(azetidin-3-ylmethyl)-2-[1-(4-ethoxy-2,6-difluorobenzyl)-1H-indazol-3-yl]-N-(pyridin-4-yl)pyrimidin-4-amine, salt with hydrochloric acid;
5-(azetidin-3-ylmethyl)-2-[1-(4-ethoxy-2,6-difluorobenzyl)-1H-indazol-3-yl]-N-(pyridin-4-yl)pyrimidin-4-amine;
5-(aminomethyl)-2-[1-(4-ethoxy-2,6-difluorobenzyl)-1H-indazol-3-yl]-N-(pyridin-4-yl)pyrimidin-4-amine, salt with hydrochloric acid;
5-(aminomethyl)-2-[1-(4-ethoxy-2,6-difluorobenzyl)-1H-indazol-3-yl]-N-(pyridin-4-yl)pyrimidin-4-amine;
5-(aminomethyl)-N-[2-(difluoromethyl)pyridin-4-yl]-2-[1-(4-ethoxy-2,6-difluorobenzyl)-1H-indazol-3-yl]pyrimidin-4-amine, salt with hydrochloric acid;
5-(aminomethyl)-N-[2-(difluoromethyl)pyridin-4-yl]-2-[1-(4-ethoxy-2,6-difluorobenzyl)-1H-indazol-3-yl]pyrimidin-4-amine;
N-[4-({5-(azetidin-3-ylmethyl)-2-[1-(4-ethoxy-2,6-difluorobenzyl)-1H-indazol-3-yl]pyrimidin-4-yl}amino)pyridin-2-yl]acetamide, salt with hydrochloric acid;
N-[4-({5-(azetidin-3-ylmethyl)-2-[1-(4-ethoxy-2,6-difluorobenzyl)-1H-indazol-3-yl]pyrimidin-4-yl}amino)pyridin-2-yl]acetamide;
5-(azetidin-3-ylmethyl)-2-[1-(4-ethoxy-2,6-difluorobenzyl)-1H-indazol-3-yl]-N-[2-trifluoromethyl)pyridin-4-yl]pyrimidin-4-amine, salt with hydrochloric acid;
5-(azetidin-3-ylmethyl)-2-[1-(4-ethoxy-2,6-difluorobenzyl)-1H-indazol-3-yl]-N-[2-trifluoromethyl)pyridin-4-yl]pyrimidin-4-amine;
5-(azetidin-3-ylmethyl)-N-[2-(difluoromethyl)pyridin-4-yl]-2-[1-(4-ethoxy-2,6-difluorobenzyl)-1H-indazol-3-yl]pyrimidin-4-amine, salt with hydrochloric acid;
5-(azetidin-3-ylmethyl)-N-[2-(difluoromethyl)pyridin-4-yl]-2-[1-(4-ethoxy-2,6-difluorobenzyl)-1H-indazol-3-yl]pyrimidin-4-amine;
5-(2-aminoethyl)-N-[2-(difluoromethyl)pyridin-4-yl]-2-[1-(4-ethoxy-2,6-difluorobenzyl)-1H-indazol-3-yl]pyrimidin-4-amine, salt with hydrochloric acid;
5-(2-aminoethyl)-N-[2-(difluoromethyl)pyridin-4-yl]-2-[1-(4-ethoxy-2,6-difluorobenzyl)-1H-indazol-3-yl]pyrimidin-4-amine;
5-(2-aminoethyl)-2-[1-(4-ethoxy-2,6-difluorobenzyl)-1H-indazol-3-yl]-N-[2-(tri-fluoromethyl)pyridin-4-yl]pyrimidin-4-amine, salt with hydrochloric acid;
5-(2-aminoethyl)-2-[1-(4-ethoxy-2,6-difluorobenzyl)-1H-indazol-3-yl]-N-[2-(tri-fluoromethyl)pyridin-4-yl]pyrimidin-4-amine;
5-(2-aminoethyl)-2-[1-(4-ethoxy-2,6-difluorobenzyl)-1H-indazol-3-yl]-N-(2-methylpyridin-4-yl)pyrimidin-4-amine, salt with hydrochloric acid;
5-(2-aminoethyl)-2-[1-(4-ethoxy-2,6-difluorobenzyl)-1H-indazol-3-yl]-N-(2-methylpyridin-4-yl)pyrimidin-4-amine;
N-[4-({5-(2-aminoethyl)-2-[1-(4-ethoxy-2,6-difluorobenzyl)-1H-indazol-3-yl]pyrimidin-4-yl}amino)pyridin-2-yl]acetamide, salt with hydrochloric acid;
N-[4-({5-(2-aminoethyl)-2-[1-(4-ethoxy-2,6-difluorobenzyl)-1H-indazol-3-yl]pyrimidin-4-yl}amino)pyridin-2-yl]acetamide;
2-[1-(4-ethoxy-2,6-difluorobenzyl)-1H-indazol-3-yl]-N-(pyridin-4-yl)-6,7-dihydro-5H-pyrrolo[3,4-d]pyrimidin-4-amine, salt with hydrochloric acid;
2-[1-(4-ethoxy-2,6-difluorobenzyl)-1H-indazol-3-yl]-N-(pyridin-4-yl)-6,7-dihydro-5H-pyrrolo[3,4-d]pyrimidin-4-amine;
2-[1-(4-ethoxy-2,6-difluorobenzyl)-1H-indazol-3-yl]-N-[2-(trifluoromethyl)pyridin-4-yl]-6,7-dihydro-5H-pyrrolo[3,4-d]pyrimidin-4-amine, salt with hydrochloric acid;
2-[1-(4-ethoxy-2,6-difluorobenzyl)-1H-indazol-3-yl]-N-[2-(trifluoromethyl)pyridin-4-yl]-6,7-dihydro-5H-pyrrolo[3,4-d]pyrimidin-4-amine;
2-[1-(4-ethoxy-2,6-difluorobenzyl)-1H-indazol-3-yl]-N-(pyridin-4-yl)-5,6,7,8-tetrahydropyrido[4,3-d]pyrimidin-4-amine;
2-[1-(4-ethoxy-2,6-difluorobenzyl)-1H-indazol-3-yl]-N-[2-(trifluoromethyl)pyridin-4-yl]-5,6,7,8-tetrahydropyrido[4,3-d]pyrimidin-4-amine;
5-(2-amino-2-methylpropyl)-2-[1-(4-ethoxy-2,6-difluorobenzyl)-1H-indazol-3-yl]-N-(pyridin-4-yl)pyrimidin-4-amine, salt with hydrochloric acid;
5-(2-amino-2-methylpropyl)-2-[1-(4-ethoxy-2,6-difluorobenzyl)-1H-indazol-3-yl]-N-(pyridin-4-yl)pyrimidin-4-amine;
4-({2-[1-(4-ethoxy-2,6-difluorobenzyl)-1H-indazol-3-yl]-5-(3-methoxypropyl)-pyrimidin-4-yl}amino)nicotinamide;
4-({2-[1-(4-ethoxy-2,6-difluorobenzyl)-1H-indazol-3-yl]-6-methylpyrimidin-4-yl}-amino)pyridine-3-carboxamide;
2-[1-(4-ethoxy-2,6-difluorobenzyl)-1H-indazol-3-yl]-N-(pyridin-4-yl)-5-{[1-(2,2,2-trifluoroethyl)azetidin-3-yl]methyl}pyrimidin-4-amine;
2-[1-(4-ethoxy-2,6-difluorobenzyl)-1H-indazol-3-yl]-N-(pyridin-4-yl)-5-[1-(2,2,2-trifluoroethyl)azetidin-3-yl]pyrimidin-4-amine;
N-({2-[1-(4-ethoxy-2,6-difluorobenzyl)-1H-indazol-3-yl]-7-(pyridin-4-yl)-6,7-dihydro-5H-pyrrolo[2,3d]pyrimidin-5-yl}methyl)-2,2,2-trifluoroethanamine;
N-{4-[(2-[1-(4-ethoxy-2,6-difluorobenzyl)-1H-indazol-3-yl]-5-{[1-(2,2,2-trifluoro-ethyl)azetidin-3-yl]methyl}pyrimidin-4-yl)amino]pyridin-2-yl}acetamide;
2-[1-(4-ethoxy-2,6-difluorobenzyl)-1H-indazol-3-yl]-6-methyl-N-(pyridin-4-yl)-pyrimidin-4-amine;
2-[1-(4-ethoxy-2,6-difluorobenzyl)-1H-indazol-3-yl]-N-(pyridin-4-yl)-6,7-dihydro-5H-cyclopenta[d]pyrimidin-4-amine;
2-[1-(4-ethoxy-2,6-difluorobenzyl)-1H-indazol-3-yl]-6-(propan-2-yl)-N-(pyridin-4-yl)pyrimidin-4-amine;
2-[1-(4-ethoxy-2,6-difluorobenzyl)-1H-indazol-3-yl]-6-(methoxymethyl)-N-(pyridin-4-yl)pyrimidin-4-amine;
2-[1-(4-ethoxy-2,6-difluorobenzyl)-1H-indazol-3-yl]-5-fluoro-6-methyl-N-(pyridin-4-yl)pyrimidin-4-amine;
2-[1-(4-ethoxy-2,6-difluorobenzyl)-1H-indazol-3-yl]-N-(2-methylpyridin-4-yl)-6,7-dihydro-5H-cyclopenta[d]pyrimidin-4-amine;
2-[1-(4-ethoxy-2,6-difluorobenzyl)-1H-indazol-3-yl]-5-ethyl-6-methyl-N-(pyridin-4-yl)pyrimidin-4-amine;
2-[1-(4-ethoxy-2,6-difluorobenzyl)-1H-indazol-3-yl]-6-ethyl-N-(pyridin-4-yl)-pyrimidin-4-amine;
2-[1-(4-ethoxy-2,6-difluorobenzyl)-1H-indazol-3-yl]-6-(morpholin-4-ylmethyl)-N-(pyridin-4-yl)pyrimidin-4-amine;
2-[1-(4-ethoxy-2,6-difluorobenzyl)-1H-indazol-3-yl]-N-(3-methylpyridin-4-yl)-6-(morpholin-4-ylmethyl)pyrimidin-4-amine;
N-[4-({2-[1-(4-ethoxy-2,6-difluorobenzyl)-1H-indazol-3-yl]-6-(morpholin-4-yl-methyl)pyrimidin-4-yl}amino)pyridin-2-yl]acetamide;
7-benzyl-2-[1-(4-ethoxy-2,6-difluorobenzyl)-1H-indazol-3-yl]-N-(pyridin-4-yl)-5,6,7,8-tetrahydropyrido[3,4-d]pyrimidin-4-amine;
7-benzyl-2-[1-(4-ethoxy-2,6-difluorobenzyl)-1H-indazol-3-yl]-N-(3-methylpyridin-4-yl)-5,6,7,8-tetrahydropyrido[3,4-d]pyrimidin-4-amine;
2-[1-(4-ethoxy-2,6-difluorobenzyl)-1H-indazol-3-yl]-6-[(methylsulfanyl)methyl]-N-(pyridin-4-yl)pyrimidin-4-amine;
N-[4-({2-[1-(4-ethoxy-2,6-difluorobenzyl)-1H-indazol-3-yl]-6,7-dihydro-5H-cyclopenta[d]pyrimidin-4-yl}amino)pyridin-2-yl]acetamide;
2-[1-(4-ethoxy-2,6-difluorobenzyl)-1H-indazol-3-yl]-6-(methoxymethyl)-N-(3-methylpyridin-4-yl)pyrimidin-4-amine;
N-[4-({2-[1-(4-ethoxy-2,6-difluorobenzyl)-1H-indazol-3-yl]-6-(methoxy-methyl)pyrimidin-4-yl}amino)pyridin-2-yl]acetamide;
2-[1-(4-ethoxy-2,6-difluorobenzyl)-1H-indazol-3-yl]-5-fluoro-6-methyl-N-[2-(tri-fluoromethyl)pyridin-4-yl]pyrimidin-4-amine;
N-[4-({2-[1-(4-ethoxy-2,6-difluorobenzyl)-1H-indazol-3-yl]-6-ethylpyrimidin-4-yl}-amino)pyridin-2-yl]acetamide;
5-chloro-2-[1-(4-ethoxy-2,6-difluorobenzyl)-1H-indazol-3-yl]-6-methyl-N-(pyridin-4-yl)pyrimidin-4-amine;
5-fluoro-2-[1-(4-methoxybenzyl)-1H-indazol-3-yl]-6-methyl-N-(pyridin-4-yl)-pyrimidin-4-amine;
2-[1-(4-ethoxy-2,6-difluorobenzyl)-1H-indazol-3-yl]-N-(6-methoxypyrimidin-4-yl)-6,7-dihydro-5H-cyclopenta[d]pyrimidin-4-amine;
N-(2,6-dimethylpyrimidin-4-yl)-2-[1-(4-ethoxy-2,6-difluorobenzyl)-1H-indazol-3-yl]-6,7-dihydro-5H-cyclopenta[d]pyrimidin-4-amine;
N-[4-({2-[1-(4-ethoxy-2,6-difluorobenzyl)-1H-indazol-3-yl]-6,7-dihydro-5H-cyclopenta[d]pyrimidin-4-yl}amino)pyridin-2-yl]cyclopropanecarboxamide;
N-(3-chloropyridin-4-yl)-2-{1-[2,6-difluoro-4-(2-methoxyethoxy)benzyl]-1H-indazol-3-yl}-6,7-dihydro-5H-cyclopenta[d]pyrimidin-4-amine;
2-{1-[2,6-difluoro-4-(2-methoxyethoxy)benzyl]-1H-indazol-3-yl}-N-(3-methoxypyridin-4-yl)-6,7-dihydro-5H-cyclopenta[d]pyrimidin-4-amine;
N-(6-chloropyrimidin-4-yl)-2-[1-(4-ethoxy-2,6-difluorobenzyl)-1H-indazol-3-yl]-6,7-dihydro-5H-cyclopenta[d]pyrimidin-4-amine;
2-[1-(4-ethoxy-2,6-difluorobenzyl)-1H-indazol-3-yl]-N-[2-(methylsulfonyl)pyridin-4-yl]-6-(morpholin-4-ylmethyl)pyrimidin-4-amine;
N-(3-chloropyridin-4-yl)-2-[1-(4-ethoxy-2,6-difluorobenzyl)-1H-indazol-3-yl]-6-(morpholin-4-ylmethyl)pyrimidin-4-amine;
N-{2-[1-(4-ethoxy-2,6-difluorobenzyl)-1H-indazol-3-yl]-6-(morpholin-4-ylmethyl)-pyrimidin-4-yl}pyridazin-4-amine;
2-{1-[2,6-difluoro-4-(2-methoxyethoxy)benzyl]-1H-indazol-3-yl}-N-[2-(difluoro-methyl)pyridin-4-yl]-6-(morpholin-4-ylmethyl)pyrimidin-4-amine;
2-{1-[2,6-difluoro-4-(2-methoxyethoxy)benzyl]-1H-indazol-3-yl}-6-(morpholin-4-yl-methyl)-N-(pyrimidin-4-yl)pyrimidin-4-amine;
2-[1-(4-ethoxy-2,6-difluorobenzyl)-1H-indazol-3-yl]-N-(5-methylpyrimidin-4-yl)-6-(morpholin-4-ylmethyl)pyrimidin-4-amine;
N-[2-{1-[2,6-difluoro-4-(2-methoxyethoxy)benzyl]-1H-indazol-3-yl}-6-(morpholin-4-ylmethyl)pyrimidin-4-yl]pyridazin-4-amine;
N-(3-chloropyridin-4-yl)-2-{1-[2,6-difluoro-4-(2-methoxyethoxy)benzyl]-1H-indazol-3-yl}-6-(morpholin-4-ylmethyl)pyrimidin-4-amine;
2-{1-[2,6-difluoro-4-(2-methoxyethoxy)benzyl]-1H-indazol-3-yl}-N-(3-methylpyridin-4-yl)-6-(morpholin-4-ylmethyl)pyrimidin-4-amine;
2-{1-[2,6-difluoro-4-(2-methoxyethoxy)benzyl]-1H-indazol-3-yl}-N-(3-methoxypyridin-4-yl)-6-(morpholin-4-ylmethyl)pyrimidin-4-amine;
2-{1-[2,6-difluoro-4-(2-methoxyethoxy)benzyl]-1H-indazol-3-yl}-6-(morpholin-4-ylmethyl)-N-(pyridin-4-yl)pyrimidin-4-amine;
N-(2,6-dimethylpyrimidin-4-yl)-2-[1-(4-ethoxy-2,6-difluorobenzyl)-1H-indazol-3-yl]-6-(morpholin-4-ylmethyl)pyrimidin-4-amine;
6-({2-[1-(4-ethoxy-2,6-difluorobenzyl)-1H-indazol-3-yl]-6-(morpholin-4-ylmethyl)-pyrimidin-4-yl}amino)pyrimidin-4-ol;
4-({2-[1-(4-ethoxy-2,6-difluorobenzyl)-1H-indazol-3-yl]-6-(morpholin-4-ylmethyl)-pyrimidin-4-yl}amino)pyridin-2-ol;
2-[1-(4-ethoxy-2,6-difluorobenzyl)-1H-indazol-3-yl]-6-(morpholin-4-ylmethyl)-N-(pyrimidin-4-yl)pyrimidin-4-amine;
2-[1-(4-ethoxy-2,6-difluorobenzyl)-1H-indazol-3-yl]-6-(methoxymethyl)-N-(2-methylpyridin-4-yl)pyrimidin-4-amine;
2-{3,5-difluoro-4-[(3-{4-(methoxymethyl)-6-[(3-methoxypyridin-4-yl)amino]-pyrimidin-2-yl}-1H-indazol-1-yl)methyl]phenoxy}ethanol;
2-{3,5-difluoro-4-[(3-{4-(methoxymethyl)-6-[(3-methylpyridin-4-yl)amino]pyrimidin-2-yl}-1H-indazol-1-yl)methyl]phenoxy}ethanol;
2-{3,5-difluoro-4-[(3-{4-(methoxymethyl)-6-[(2-methylpyridin-4-yl)amino]pyrimidin-2-yl}-1H-indazol-1-yl)methyl]phenoxy}ethanol;
2-[1-(4-ethoxy-2,6-difluorobenzyl)-1H-indazol-3-yl]-N-(1,3,5-triazin-2-yl)-6,7-dihydro-5H-cyclopenta[d]pyrimidin-4-amine;
N-(6-chloropyridazin-4-yl)-2-[1-(4-ethoxy-2,6-difluorobenzyl)-1H-indazol-3-yl]-6,7-dihydro-5H-cyclopenta[d]pyrimidin-4-amine;
2-[1-(4-ethoxy-2,6-difluorobenzyl)-1H-indazol-3-yl]-N-(2-methylpyrimidin-4-yl)-6,7-dihydro-5H-cyclopenta[d]pyrimidin-4-amine;
3-{2-[1-(4-ethoxy-2,6-difluorobenzyl)-1H-indazol-3-yl]-4-(pyridin-4-ylamino)-pyrimidin-5-yl}propan-1-ol;
2-[1-(4-ethoxy-2,6-difluorobenzyl)-1H-indazol-3-yl]-N-(3-fluoropyridin-4-yl)-6,7-dihydro-5H-cyclopenta[d]pyrimidin-4-amine;
2-[1-(4-ethoxy-2,6-difluorobenzyl)-1H-indazol-3-yl]-N-(3-methylpyridin-4-yl)-6,7-dihydro-5H-cyclopenta[d]pyrimidin-4-amine;
2-[1-(4-ethoxy-2,6-difluorobenzyl)-1H-indazol-3-yl]-N-[3-(trifluoromethyl)pyridin-4-yl]-6,7-dihydro-5H-cyclopenta[d]pyrimidin-4-amine;
2-[1-(4-ethoxy-2,6-difluorobenzyl)-1H-indazol-3-yl]-N-(3-methoxypyridin-4-yl)-6,7-dihydro-5H-cyclopenta[d]pyrimidin-4-amine;
2-[1-(4-ethoxy-2,6-difluorobenzyl)-1H-indazol-3-yl]-N-(pyridazin-4-yl)-6,7-dihydro-5H-cyclopenta[d]pyrimidin-4-amine;
2-[1-(4-ethoxy-2,6-difluorobenzyl)-1H-indazol-3-yl]-N-(pyrimidin-4-yl)-6,7-dihydro-5H-cyclopenta[d]pyrimidin-4-amine;
N-(3-chloropyridin-4-yl)-2-[1-(4-ethoxy-2,6-difluorobenzyl)-1H-indazol-3-yl]-6,7-dihydro-5H-cyclopenta[d]pyrimidin-4-amine;
N-(3-cyclopropylpyridin-4-yl)-2-[1-(4-ethoxy-2,6-difluorobenzyl)-1H-indazol-3-yl]-6,7-dihydro-5H-cyclopenta[d]pyrimidin-4-amine;
4-({2-[1-(4-ethoxy-2,6-difluorobenzyl)-1H-indazol-3-yl]-6,7-dihydro-5H-cyclopenta[d]pyrimidin-4-yl}amino)pyridine-3-carboxylic acid;
2-[1-(4-ethoxy-2,6-difluorobenzyl)-1H-indazol-3-yl]-N-(pyridin-4-yl)-5,7-dihydrofuro[3,4-d]pyrimidin-4-amine;
2-[1-(4-ethoxy-2,6-difluorobenzyl)-1H-indazol-3-yl]-6-ethyl-N-(pyridin-4-yl)-5,6,7,8-tetrahydropyrido[4,3-d]pyrimidin-4-amine;
2-[1-(4-ethoxy-2,6-difluorobenzyl)-1H-indazol-3-yl]-6-ethyl-N-(2-methylpyridin-4-yl)-5,6,7,8-tetrahydropyrido[4,3-d]pyrimidin-4-amine;
2-[1-(4-ethoxy-2,6-difluorobenzyl)-1H-indazol-3-yl]-6-(piperidin-1-ylmethyl)-N-(pyridin-4-yl)pyrimidin-4-amine;
2-[1-(4-ethoxy-2,6-difluorobenzyl)-1H-indazol-3-yl]-N-(pyridin-4-yl)-5-{2-[(2,2,2-trifluoroethyl)amino]ethyl}pyrimidin-4-amine;
2-[1-(4-ethoxy-2,6-difluorobenzyl)-1H-indazol-3-yl]-5-(piperidin-4-yl)-N-(pyridin-4-yl)pyrimidin-4-amine;
N-[4-({2-[1-(4-ethoxy-2,6-difluorobenzyl)-1H-indazol-3-yl]-5-(piperidin-4-yl)-pyrimidin-4-yl}amino)pyridin-2-yl]acetamide;
2-[1-(4-ethoxy-2,6-difluorobenzyl)-1H-indazol-3-yl]-N-(2-methylpyrimidin-4-yl)-5-(piperidin-4-yl)pyrimidin-4-amine;
2-[1-(4-ethoxy-2,6-difluorobenzyl)-1H-indazol-3-yl]-5-(piperidin-4-yl)-N-(pyrimidin-4-yl)pyrimidin-4-amine;
2-[1-(4-ethoxy-2,6-difluorobenzyl)-1H-indazol-3-yl]-N-(pyridin-4-yl)-5,6,7,8-tetrahydropyrido[3,4-d]pyrimidin-4-amine;
2-[1-(4-ethoxy-2,6-difluorobenzyl)-1H-indazol-3-yl]-N-(pyridin-4-yl)-5,7-dihydrothieno[3,4-d]pyrimidin-4-amine;
2-[1-(4-ethoxy-2,6-difluorobenzyl)-1H-indazol-3-yl]-N-(3-methylpyridin-4-yl)-5,7-dihydrothieno[3,4-d]pyrimidin-4-amine;
N-[4-({2-[1-(4-ethoxy-2,6-difluorobenzyl)-1H-indazol-3-yl]-5,7-dihydrothieno[3,4-d]pyrimidin-4-yl}amino)pyridin-2-yl]acetamide;
2-[1-(4-ethoxy-2,6-difluorobenzyl)-1H-indazol-3-yl]-N-(2-methylpyridin-4-yl)-5,7-dihydrothieno[3,4-d]pyrimidin-4-amine;
2-[1-(4-ethoxy-2,6-difluorobenzyl)-1H-indazol-3-yl]-N-(2-methylpyridin-4-yl)-5,7-dihydrothieno[3,4-d]pyrimidin-4-amine 6,6-dioxide;
2-[1-(4-ethoxy-2,6-difluorobenzyl)-1H-indazol-3-yl]-N-[2-(methylsulfonyl)pyridin-4-yl]-6,7-dihydro-5H-cyclopenta[d]pyrimidin-4-amine;
2-[1-(4-ethoxy-2,6-difluorobenzyl)-1H-indazol-3-yl]-N-(2-methoxypyridin-4-yl)-6,7-dihydro-5H-cyclopenta[d]pyrimidin-4-amine;
N-[2-(difluoromethyl)pyridin-4-yl]-2-[1-(4-ethoxy-2,6-difluorobenzyl)-1H-indazol-3-yl]-6,7-dihydro-5H-cyclopenta[d]pyrimidin-4-amine;
2-[1-(4-ethoxy-2,6-difluorobenzyl)-1H-indazol-3-yl]-N-(2-methylpyridin-4-yl)-6-(morpholin-4-ylmethyl)pyrimidin-4-amine;
2-[1-(4-ethoxy-2,6-difluorobenzyl)-1H-indazol-3-yl]-N-(2-methoxypyridin-4-yl)-6-(morpholin-4-ylmethyl)pyrimidin-4-amine;
N-[4-({2-[1-(4-ethoxy-2,6-difluorobenzyl)-1H-indazol-3-yl]-6-(morpholin-4-yl-methyl)pyrimidin-4-yl}amino)pyridin-2-yl]cyclopropanecarboxamide;
2-[1-(4-ethoxy-2,6-difluorobenzyl)-1H-indazol-3-yl]-N-(3-fluoropyridin-4-yl)-6-(morpholin-4-ylmethyl)pyrimidin-4-amine;
2-[1-(4-ethoxy-2,6-difluorobenzyl)-1H-indazol-3-yl]-N-(3-methoxypyridin-4-yl)-6-(morpholin-4-ylmethyl)pyrimidin-4-amine;
N-[2-(difluoromethyl)pyridin-4-yl]-2-[1-(4-ethoxy-2,6-difluorobenzyl)-1H-indazol-3-yl]-5,7-dihydrothieno[3,4-d]pyrimidin-4-amine 6,6-dioxide;
2-[1-(4-ethoxy-2,6-difluorobenzyl)-1H-indazol-3-yl]-N-(2-methylpyridin-4-yl)-5,7-dihydrofuro[3,4-d]pyrimidin-4-amine;
2-[1-(4-ethoxy-2,6-difluorobenzyl)-1H-indazol-3-yl]-N-(3-methoxypyridin-4-yl)-5,7-dihydrofuro[3,4-d]pyrimidin-4-amine;
2-[1-(4-ethoxy-2,6-difluorobenzyl)-1H-indazol-3-yl]-N-(3-methylpyridin-4-yl)-5,7-dihydrofuro[3,4-d]pyrimidin-4-amine;
N-[2-(difluoromethyl)pyridin-4-yl]-2-[1-(4-ethoxy-2,6-difluorobenzyl)-1H-indazol-3-yl]-5,6,7,8-tetrahydroquinazolin-4-amine;
2-[1-(4-ethoxy-2,6-difluorobenzyl)-1H-indazol-3-yl]-N-(3-methylpyridin-4-yl)-5,6,7,8-tetrahydroquinazolin-4-amine;
2-[1-(4-ethoxy-2,6-difluorobenzyl)-1H-indazol-3-yl]-N-(pyridin-4-yl)-5,6,7,8-tetrahydroquinazolin-4-amine;
N-[4-({2-[1-(4-ethoxy-2,6-difluorobenzyl)-1H-indazol-3-yl]-5-[1-(2,2,2-trifluoro-ethyl)piperidin-4-yl]pyrimidin-4-yl}amino)pyridin-2-yl]acetamide;
2-[1-(4-ethoxy-2,6-difluorobenzyl)-1H-indazol-3-yl]-N-(2-methylpyrimidin-4-yl)-5-[1-(2,2,2-trifluoroethyl)piperidin-4-yl]pyrimidin-4-amine;
2-[1-(4-ethoxy-2,6-difluorobenzyl)-1H-indazol-3-yl]-N-(pyrimidin-4-yl)-5-[1-(2,2,2-trifluoroethyl)piperidin-4-yl]pyrimidin-4-amine;
2-[1-(4-ethoxy-2,6-difluorobenzyl)-1H-indazol-3-yl]-N-(6-methylpyrimidin-4-yl)-6,7-dihydro-5H-cyclopenta[d]pyrimidin-4-amine;
2-{1-[2,6-difluoro-4-(2-methoxyethoxy)benzyl]-1H-indazol-3-yl}-N-(3-methoxypyridin-4-yl)-5-methylpyrimidin-4-amine;
5-cyclopropyl-2-{1-[2,6-difluoro-4-(2-methoxyethoxy)benzyl]-1H-indazol-3-yl}-N-(3-methoxypyridin-4-yl)pyrimidin-4-amine;
N-{4-[(2-{1-[2,6-difluoro-4-(2-hydroxyethoxy)benzyl]-1H-indazol-3-yl}-5-[1-(2,2,2-trifluoroethyl)piperidin-4-yl]pyrimidin-4-yl)amino]pyridin-2-yl}acetamide;
2-{4-[(3-{4-[(3-chloropyridin-4-yl)amino]-5-[1-(2,2,2-trifluoroethyl)piperidin-4-yl]-pyrimidin-2-yl}-1H-indazol-1-yl)methyl]-3,5-difluorophenoxy}ethanol;
2-{4-[(3-{4-[(3-chloropyridin-4-yl)amino]-5-[1-(3,3,3-trifluoropropyl)piperidin-4-yl]-pyrimidin-2-yl}-1H-indazol-1-yl)methyl]-3,5-difluorophenoxy}ethanol;
2-{3,5-difluoro-4-[(3-{4-[(2-methylpyrimidin-4-yl)amino]-5-[1-(2,2,2-trifluoroethyl)-piperidin-4-yl]pyrimidin-2-yl}-1H-indazol-1-yl)methyl]phenoxy}ethanol;
2-{4-[(3-{5-[1-(2,2-difluoroethyl)piperidin-4-yl]-4-[(2-methylpyrimidin-4-yl)amino]-pyrimidin-2-yl}-1H-indazol-1-yl)methyl]-3,5-difluorophenoxy}ethanol;
2-{3,5-difluoro-4-[(3-{4-[(2-methylpyrimidin-4-yl)amino]-6,7-dihydro-5H-cyclopenta[d]pyrimidin-2-yl}-1H-indazol-1-yl)methyl]phenoxy}ethanol;
2-[3,5-difluoro-4-({3-[4-(pyrimidin-4-ylamino)-6,7-dihydro-5H-cyclopenta[d]pyrimidin-2-yl]-1H-indazol-1-yl}methyl)phenoxy]ethanol;
2-{4-[(3-{4-[(2,5-dimethylpyridin-4-yl)amino]-6,7-dihydro-5H-cyclopenta[d]pyrimidin-2-yl}-1H-indazol-1-yl)methyl]-3,5-difluorophenoxy}ethanol;
2-{3,5-difluoro-4-[(3-{4-[(5-fluoro-2-methylpyridin-4-yl)amino]-6,7-dihydro-5H-cyclopenta[d]pyrimidin-2-yl}-1H-indazol-1-yl)methyl]phenoxy}ethanol;
2-{4-[(3-{6,6-dimethyl-4-[(2-methylpyrimidin-4-yl)amino]-6,7-dihydro-5H-cyclopenta[d]pyrimidin-2-yl}-1H-indazol-1-yl)methyl]-3,5-difluorophenoxy}ethanol;
2-{4-[(3-{5-[1-(cyclopropylmethyl)piperidin-4-yl]-4-[(2-methylpyrimidin-4-yl)amino]-pyrimidin-2-yl}-1H-indazol-1-yl)methyl]-3,5-difluorophenoxy}ethanol;
2-{4-[(3-{7,7-dimethyl-4-[(2-methylpyrimidin-4-yl)amino]-5,6,7,8-tetrahydroquinazolin-2-yl}-1H-indazol-1-yl)methyl]-3,5-difluorophenoxy}ethanol;
2-{4-[(3-{4-[(3-chloropyridin-4-yl)amino]-7,7-dimethyl-5,6,7,8-tetrahydroquinazolin-2-yl}-1H-indazol-1-yl)methyl]-3,5-difluorophenoxy}ethanol;
2-{3,5-difluoro-4-[(3-{4-[(3-methoxypyridin-4-yl)amino]-7,7-dimethyl-5,6,7,8-tetrahydroquinazolin-2-yl}-1H-indazol-1-yl)methyl]phenoxy}ethanol;
2-{3,5-difluoro-4-[(3-{4-methyl-6-[(2-methylpyrimidin-4-yl)amino]pyrimidin-2-yl}-1H-indazol-1-yl)methyl]phenoxy}ethanol;
2-{3,5-difluoro-4-[(3-{4-isopropyl-6-[(2-methylpyrimidin-4-yl)amino]pyrimidin-2-yl}-1H-indazol-1-yl)methyl]phenoxy}ethanol;
2-{3,5-difluoro-4-[(3-{4-isopropyl-6-[(3-methoxypyridin-4-yl)amino]pyrimidin-2-yl}-1H-indazol-1-yl)methyl]phenoxy}ethanol;
2-{4-[(3-{4-[(2,5-dimethylpyridin-4-yl)amino]-6-isopropylpyrimidin-2-yl}-1H-indazol-1-yl)methyl]-3,5-difluorophenoxy}ethanol;
2-{3,5-difluoro-4-[(3-{4-[(3-methoxypyridin-4-yl)amino]-5,6-dimethylpyrimidin-2-yl}-1H-indazol-1-yl)methyl]phenoxy}ethanol;
2-{3,5-difluoro-4-[(3-{5-fluoro-4-methyl-6-[(2-methylpyrimidin-4-yl)amino]pyrimidin-2-yl}-1H-indazol-1-yl)methyl]phenoxy}ethanol;
2-{3,5-difluoro-4-[(3-{4-[(3-methoxypyridin-4-yl)amino]-6-methylpyrimidin-2-yl}-1H-indazol-1-yl)methyl]phenoxy}ethanol;
2-{3,5-difluoro-4-[(3-{4-[(3-methoxypyridin-4-yl)amino]-5-methylpyrimidin-2-yl}-1H-indazol-1-yl)methyl]phenoxy}ethanol;
2-(3,5-difluoro-4-{[3-(4-methyl-6-{[3-(trifluoromethoxy)pyridin-4-yl]amino}pyrimidin-2-yl)-1H-indazol-1-yl]methyl}phenoxy)ethanol;
2-{4-[(3-{5-cyclopropyl-4-[(3-methoxypyridin-4-yl)amino]pyrimidin-2-yl}-1H-indazol-1-yl)methyl]-3,5-difluorophenoxy}ethanol;
2-[3,5-difluoro-4-({3-[4-(morpholin-4-ylmethyl)-6-(pyridazin-4-ylamino)pyrimidin-2-yl]-1H-indazol-1-yl}methyl)phenoxy]ethanol;
2-[3,5-difluoro-4-({3-[4-(morpholin-4-ylmethyl)-6-(pyridin-4-ylamino)pyrimidin-2-yl]-1H-indazol-1-yl}methyl)phenoxy]ethanol;
2-{3,5-difluoro-4-[(3-{4-[(2-methylpyrimidin-4-yl)amino]-6-(2,2,2-trifluoroethyl)-6,7-dihydro-5H-pyrrolo[3,4-d]pyrimidin-2-yl}-1H-indazol-1-yl)methyl]phenoxy}ethanol;
2-[3,5-difluoro-4-({3-[4-(pyrimidin-4-ylamino)-6-(2,2,2-trifluoroethyl)-6,7-dihydro-5H-pyrrolo[3,4-d]pyrimidin-2-yl]-1H-indazol-1-yl}methyl)phenoxy]ethanol;
2-{3,5-difluoro-4-[(3-{4-[(3-methoxypyridin-4-yl)amino]-6-(2,2,2-trifluoroethyl)-6,7-dihydro-5H-pyrrolo[3,4-d]pyrimidin-2-yl}-1H-indazol-1-yl)methyl]phenoxy}ethanol;
N-{4-[(2-{1-[2,6-difluoro-4-(2-hydroxyethoxy)benzyl]-1H-indazol-3-yl}-7,7-dimethyl-5,6,7,8-tetrahydroquinazolin-4-yl)amino]pyridin-2-yl}acetamide;
2-{4-[(3-{4-[(2,5-dimethylpyridin-4-yl)amino]-7,7-dimethyl-5,6,7,8-tetrahydroquinazolin-2-yl}-1H-indazol-1-yl)methyl]-3,5-difluorophenoxy}ethanol;
2-{3,5-difluoro-4-[(3-{4-[(3-methoxypyridin-4-yl)amino]-6,7,8,9-tetrahydro-5H-cyclohepta[d]pyrimidin-2-yl}-1H-indazol-1-yl)methyl]phenoxy}ethanol;
5-(azetidin-3-ylmethyl)-N-(2,5-dimethylpyridin-4-yl)-2-[1-(4-ethoxy-2,6-difluorobenzyl)-1H-indazol-3-yl]pyrimidin-4-amine, salt with hydrochloric acid;
5-(azetidin-3-ylmethyl)-2-[1-(4-ethoxy-2,6-difluorobenzyl)-1H-indazol-3-yl]-N-(2-methylpyrimidin-4-yl)pyrimidin-4-amine, salt with hydrochloric acid;
5-(2-aminoethyl)-N-(3-chloropyridin-4-yl)-2-[1-(4-ethoxy-2,6-difluorobenzyl)-1H-indazol-3-yl]pyrimidin-4-amine;
5-(2-aminoethyl)-2-[1-(4-ethoxy-2,6-difluorobenzyl)-1H-indazol-3-yl]-N-(2-methylpyrimidin-4-yl)pyrimidin-4-amine, salt with hydrochloric acid;
5-(2-aminoethyl)-2-[1-(4-ethoxy-2,6-difluorobenzyl)-1H-indazol-3-yl]-N-(2-methylpyrimidin-4-yl)pyrimidin-4-amine;
N-(3-chloropyridin-4-yl)-2-{1-[2,6-difluoro-4-(2-methoxyethoxy)benzyl]-1H-indazol-3-yl}-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(2-methylpyrimidin-4-yl)-6,7-dihydro-5H-pyrrolo[3,4-d]pyrimidin-4-amine, salt with hydrochloric acid;
2-{1-[2,6-difluoro-4-(2-methoxyethoxy)benzyl]-1H-indazol-3-yl}-N-(2-methylpyrimidin-4-yl)-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(2,5-dimethylpyridin-4-yl)-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(pyrimidin-4-yl)-6,7-dihydro-5H-pyrrolo[3,4-d]pyrimidin-4-amine, salt with hydrochloric acid;
2-{1-[2,6-difluoro-4-(2-methoxyethoxy)benzyl]-1H-indazol-3-yl}-N-(pyrimidin-4-yl)-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(3-methoxypyridin-4-yl)-6,7-dihydro-5H-pyrrolo[3,4-d]pyrimidin-4-amine;
5-[1-(2,2-difluoroethyl)piperidin-4-yl]-2-[1-(4-ethoxy-2,6-difluorobenzyl)-1H-indazol-3-yl]-N-(3-methoxypyridin-4-yl)pyrimidin-4-amine;
2-{1-[2,6-difluoro-4-(2-methoxyethoxy)benzyl]-1H-indazol-3-yl}-N-(2-methylpyrimidin-4-yl)-6,7-dihydro-5H-cyclopenta[d]pyrimidin-4-amine;
2-{1-[2,6-difluoro-4-(2-methoxyethoxy)benzyl]-1H-indazol-3-yl}-N-(pyrimidin-4-yl)-6,7-dihydro-5H-cyclopenta[d]pyrimidin-4-amine;
2-{1-[2,6-difluoro-4-(2-methoxyethoxy)benzyl]-1H-indazol-3-yl}-N-(2,5-dimethylpyridin-4-yl)-6,7-dihydro-5H-cyclopenta[d]pyrimidin-4-amine;
2-{1-[2,6-difluoro-4-(2-methoxyethoxy)benzyl]-1H-indazol-3-yl}-N-(5-fluoro-2-methylpyridin-4-yl)-6,7-dihydro-5H-cyclopenta[d]pyrimidin-4-amine;
N-(2,5-dimethylpyridin-4-yl)-2-[1-(4-ethoxy-2,6-difluorobenzyl)-1H-indazol-3-yl]-6,6-dimethyl-6,7-dihydro-5H-cyclopenta[d]pyrimidin-4-amine;
2-[1-(4-ethoxy-2,6-difluorobenzyl)-1H-indazol-3-yl]-6,6-dimethyl-N-(2-methylpyrimidin-4-yl)-6,7-dihydro-5H-cyclopenta[d]pyrimidin-4-amine;
2-{1-[2,6-difluoro-4-(2-methoxyethoxy)benzyl]-1H-indazol-3-yl}-6,6-dimethyl-N-(2-methylpyrimidin-4-yl)-6,7-dihydro-5H-cyclopenta[d]pyrimidin-4-amine;
N-[4-({2-[1-(4-ethoxy-2,6-difluorobenzyl)-1H-indazol-3-yl]-6,6-dimethyl-6,7-dihydro-5H-cyclopenta[d]pyrimidin-4-yl}amino)pyridin-2-yl]acetamide;
2-[1-(4-ethoxy-2,6-difluorobenzyl)-1H-indazol-3-yl]-6,6-dimethyl-N-(2-methylpyrimidin-4-yl)-5,6,7,8-tetrahydroquinazolin-4-amine;
2-[1-(4-ethoxy-2,6-difluorobenzyl)-1H-indazol-3-yl]-N-(3-methoxypyridin-4-yl)-6,6-dimethyl-5,6,7,8-tetrahydroquinazolin-4-amine;
N-(2,5-dimethylpyridin-4-yl)-2-[1-(4-ethoxy-2,6-difluorobenzyl)-1H-indazol-3-yl]-6,6-dimethyl-5,6,7,8-tetrahydroquinazolin-4-amine;
N-[4-({2-[1-(4-ethoxy-2,6-difluorobenzyl)-1H-indazol-3-yl]-6,6-dimethyl-5,6,7,8-tetrahydroquinazolin-4-yl}amino)pyridin-2-yl]acetamide;
2-{1-[2,6-difluoro-4-(2-methoxyethoxy)benzyl]-1H-indazol-3-yl}-N-(3-methoxypyridin-4-yl)-6,7,8,9-tetrahydro-5H-cyclohepta[d]pyrimidin-4-amine;
2-{1-[2,6-difluoro-4-(2-methoxyethoxy)benzyl]-1H-indazol-3-yl}-6-methyl-N-(2-methylpyrimidin-4-yl)pyrimidin-4-amine;
2-{1-[2,6-difluoro-4-(2-methoxyethoxy)benzyl]-1H-indazol-3-yl}-6-isopropyl-N-(2-methylpyrimidin-4-yl)pyrimidin-4-amine;
2-{1-[2,6-difluoro-4-(2-methoxyethoxy)benzyl]-1H-indazol-3-yl}-6-isopropyl-N-(3-methoxypyridin-4-yl)pyrimidin-4-amine;
2-{1-[2,6-difluoro-4-(2-methoxyethoxy)benzyl]-1H-indazol-3-yl}-N-(2,5-dimethylpyridin-4-yl)-6-isopropylpyrimidin-4-amine;
2-{1-[2,6-difluoro-4-(2-methoxyethoxy)benzyl]-1H-indazol-3-yl}-5-fluoro-6-methyl-N-(2-methylpyrimidin-4-yl)pyrimidin-4-amine;
2-{1-[2,6-difluoro-4-(2-methoxyethoxy)benzyl]-1H-indazol-3-yl}-N-(3-methoxypyridin-4-yl)-5,6-dimethylpyrimidin-4-amine;
2-{1-[2,6-difluoro-4-(2-methoxyethoxy)benzyl]-1H-indazol-3-yl}-N-(3-methoxypyridin-4-yl)-6-methylpyrimidin-4-amine;
2-{1-[2,6-difluoro-4-(2-methoxyethoxy)benzyl]-1H-indazol-3-yl}-6-methyl-N-[3-(trifluoromethoxy)pyridin-4-yl]pyrimidin-4-amine;
2-[1-(4-ethoxy-2,6-difluorobenzyl)-1H-indazol-3-yl]-6-methyl-N-[3-(trifluoromethoxy)pyridin-4-yl]pyrimidin-4-amine;
4-({2-[1-(4-ethoxy-2,6-difluorobenzyl)-1H-indazol-3-yl]-6-methylpyrimidin-4-yl}amino)pyridin-3-ol;
2-[1-(4-ethoxy-2,6-difluorobenzyl)-1H-indazol-3-yl]-N-(3-methoxypyridin-4-yl)-6-(trifluoromethyl)pyrimidin-4-amine;
N-(2,5-dimethylpyridin-4-yl)-2-[1-(4-ethoxy-2,6-difluorobenzyl)-1H-indazol-3-yl]-6-(trifluoromethyl)pyrimidin-4-amine;
2-[1-(4-ethoxy-2,6-difluorobenzyl)-1H-indazol-3-yl]-N-(2-methylpyrimidin-4-yl)-6-(trifluoromethyl)pyrimidin-4-amine;
6-(2,4-dimethyl-1,3-thiazol-5-yl)-2-[1-(4-ethoxy-2,6-difluorobenzyl)-1H-indazol-3-yl]-N-(2-methylpyrimidin-4-yl)pyrimidin-4-amine;
6-(2,4-dimethyl-1,3-thiazol-5-yl)-2-[1-(4-ethoxy-2,6-difluorobenzyl)-1H-indazol-3-yl]-N-(pyrimidin-4-yl)pyrimidin-4-amine;
2-[1-(4-ethoxy-2,6-difluorobenzyl)-1H-indazol-3-yl]-N-(pyrimidin-4-yl)-6-[(2rac)-tetrahydrofuran-2-yl]pyrimidin-4-amine;
2-[1-(4-ethoxy-2,6-difluorobenzyl)-1H-indazol-3-yl]-N-(2-methylpyrimidin-4-yl)-6-[(2rac)-tetrahydrofuran-2-yl]pyrimidin-4-amine;
2-[1-(4-ethoxy-2,6-difluorobenzyl)-1H-indazol-3-yl]-N-(3-methoxypyridin-4-yl)-6-[(2rac)-tetrahydrofuran-2-yl]pyrimidin-4-amine;
2-[1-(4-ethoxy-2,6-difluorobenzyl)-1H-indazol-3-yl]-N-(2-methylpyrimidin-4-yl)-6-(tetrahydro-2H-pyran-4-yl)pyrimidin-4-amine;
N-[4-({2-[1-(4-ethoxy-2,6-difluorobenzyl)-1H-indazol-3-yl]-6-(tetrahydro-2H-pyran-4-yl)pyrimidin-4-yl}amino)pyridin-2-yl]acetamide;
2-[1-(4-ethoxy-2,6-difluorobenzyl)-1H-indazol-3-yl]-N-(3-methoxypyridin-4-yl)-6-(tetrahydro-2H-pyran-4-yl)pyrimidin-4-amine;
2-[1-(4-ethoxy-2,6-difluorobenzyl)-1H-indazol-3-yl]-N-(pyrimidin-4-yl)-6-(tetrahydro-2H-pyran-4-yl)pyrimidin-4-amine;
2-[1-(4-ethoxy-2,6-difluorobenzyl)-1H-indazol-3-yl]-6-(1-methyl-1H-pyrazol-3-yl)-N-(2-methylpyrimidin-4-yl)pyrimidin-4-amine;
2-[1-(4-ethoxy-2,6-difluorobenzyl)-1H-indazol-3-yl]-N-(3-methoxypyridin-4-yl)-6-(1-methyl-1H-pyrazol-3-yl)pyrimidin-4-amine;
2-[1-(4-ethoxy-2,6-difluorobenzyl)-1H-indazol-3-yl]-N-(pyrimidin-4-yl)-6-(1,3-thiazol-2-yl)pyrimidin-4-amine;
2-[1-(4-ethoxy-2,6-difluorobenzyl)-1H-indazol-3-yl]-N-(2-methylpyrimidin-4-yl)-6-(1,3-thiazol-2-yl)pyrimidin-4-amine;
2-{1-[2,6-difluoro-4-(2-methoxyethoxy)benzyl]-1H-indazol-3-yl}-N-(2-methylpyrimidin-4-yl)-7,8-dihydro-5H-pyrano[4,3-d]pyrimidin-4-amine;
2-{1-[2,6-difluoro-4-(2-methoxyethoxy)benzyl]-1H-indazol-3-yl}-7,7-dimethyl-N-(2-methylpyrimidin-4-yl)-5,6,7,8-tetrahydroquinazolin-4-amine;
N-(3-chloropyridin-4-yl)-2-{1-[2,6-difluoro-4-(2-methoxyethoxy)benzyl]-1H-indazol-3-yl}-7,7-dimethyl-5,6,7,8-tetrahydroquinazolin-4-amine;
2-{1-[2,6-difluoro-4-(2-methoxyethoxy)benzyl]-1H-indazol-3-yl}-N-(3-methoxypyridin-4-yl)-7,7-dimethyl-5,6,7,8-tetrahydroquinazolin-4-amine;
N-{4-[(2-{1-[2,6-difluoro-4-(2-methoxyethoxy)benzyl]-1H-indazol-3-yl}-7,7-dimethyl-5,6,7,8-tetrahydroquinazolin-4-yl)amino]pyridin-2-yl}acetamide;
2-{1-[2,6-difluoro-4-(2-methoxyethoxy)benzyl]-1H-indazol-3-yl}-N-(2,5-dimethylpyridin-4-yl)-7,7-dimethyl-5,6,7,8-tetrahydroquinazolin-4-amine;
2-[1-(4-ethoxy-2,6-difluorobenzyl)-1H-indazol-3-yl]-N-(3-methoxypyridin-4-yl)-5-(piperidin-4-yl)pyrimidin-4-amine;
2-[1-(4-ethoxy-2,6-difluorobenzyl)-1H-indazol-3-yl]-N-(2-methylpyridin-4-yl)-5-(piperidin-4-yl)pyrimidin-4-amine;
N-(2,5-dimethylpyridin-4-yl)-2-[1-(4-ethoxy-2,6-difluorobenzyl)-1H-indazol-3-yl]-5-(piperidin-4-yl)pyrimidin-4-amine;
N-(4-{[2-{1-[2,6-difluoro-4-(2-methoxyethoxy)benzyl]-1H-indazol-3-yl}-5-(piperidin-4-yl)pyrimidin-4-yl]amino}pyridin-2-yl)acetamide;
N-(3-chloropyridin-4-yl)-2-{1-[2,6-difluoro-4-(2-methoxyethoxy)benzyl]-1H-indazol-3-yl}-5-(piperidin-4-yl)pyrimidin-4-amine;
2-{1-[2,6-difluoro-4-(2-methoxyethoxy)benzyl]-1H-indazol-3-yl}-N-(2-methylpyrimidin-4-yl)-5-(piperidin-4-yl)pyrimidin-4-amine;
2-[1-(4-ethoxy-2,6-difluorobenzyl)-1H-indazol-3-yl]-N-(3-methoxypyridin-4-yl)-5-[1-(2,2,2-trifluoroethyl)piperidin-4-yl]pyrimidin-4-amine;
2-[1-(4-ethoxy-2,6-difluorobenzyl)-1H-indazol-3-yl]-N-(2-methylpyridin-4-yl)-5-[1-(2,2,2-trifluoroethyl)piperidin-4-yl]pyrimidin-4-amine;
N-(2,5-dimethylpyridin-4-yl)-2-[1-(4-ethoxy-2,6-difluorobenzyl)-1H-indazol-3-yl]-5-[1-(2,2,2-trifluoroethyl)piperidin-4-yl]pyrimidin-4-amine;
N-{4-[(2-{1-[2,6-difluoro-4-(2-methoxyethoxy)benzyl]-1H-indazol-3-yl}-5-[1-(2,2,2-trifluoroethyl)piperidin-4-yl]pyrimidin-4-yl)amino]pyridin-2-yl}acetamide;
N-(3-chloropyridin-4-yl)-2-{1-[2,6-difluoro-4-(2-methoxyethoxy)benzyl]-1H-indazol-3-yl}-5-[1-(2,2,2-trifluoroethyl)piperidin-4-yl]pyrimidin-4-amine;
N-(3-chloropyridin-4-yl)-2-{1-[2,6-difluoro-4-(2-methoxyethoxy)benzyl]-1H-indazol-3-yl}-5-[1-(3,3,3-trifluoropropyl)piperidin-4-yl]pyrimidin-4-amine;
2-{1-[2,6-difluoro-4-(2-methoxyethoxy)benzyl]-1H-indazol-3-yl}-N-(2-methylpyrimidin-4-yl)-5-[1-(2,2,2-trifluoroethyl)piperidin-4-yl]pyrimidin-4-amine;
5-[1-(2,2-difluoroethyl)piperidin-4-yl]-2-{1-[2,6-difluoro-4-(2-methoxyethoxy)benzyl]-1H-indazol-3-yl}-N-(2-methylpyrimidin-4-yl)pyrimidin-4-amine;
5-[1-(cyclopropylmethyl)piperidin-4-yl]-2-{1-[2,6-difluoro-4-(2-methoxyethoxy)benzyl]-1H-indazol-3-yl}-N-(2-methylpyrimidin-4-yl)pyrimidin-4-amine;
2-[1-(4-ethoxy-2,6-difluorobenzyl)-1H-indazol-3-yl]-N-(2-methylpyrimidin-4-yl)-5-{2-[(2,2,2-trifluoroethyl)amino]ethyl}pyrimidin-4-amine;
N-(3-chloropyridin-4-yl)-2-[1-(4-ethoxy-2,6-difluorobenzyl)-1H-indazol-3-yl]-5-{2-[(2,2,2-trifluoroethyl)amino]ethyl}pyrimidin-4-amine;
N-{4-[(2-[1-(4-ethoxy-2,6-difluorobenzyl)-1H-indazol-3-yl]-5-{2-[(2,2,2-trifluoroethyl)amino]ethyl}pyrimidin-4-yl)amino]pyridin-2-yl}acetamide;
2-{1-[2,6-difluoro-4-(2-methoxyethoxy)benzyl]-1H-indazol-3-yl}-N-(2-methylpyrimidin-4-yl)-6-(2,2,2-trifluoroethyl)-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(2,5-dimethylpyridin-4-yl)-6-(2,2,2-trifluoroethyl)-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(pyrimidin-4-yl)-6-(2,2,2-trifluoroethyl)-6,7-dihydro-5H-pyrrolo[3,4-d]pyrimidin-4-amine;
2-{1-[2,6-difluoro-4-(2-methoxyethoxy)benzyl]-1H-indazol-3-yl}-N-(3-methoxypyridin-4-yl)-6-(2,2,2-trifluoroethyl)-6,7-dihydro-5H-pyrrolo[3,4-d]pyrimidin-4-amine; and
2-[1-(4-ethoxy-2,6-difluorobenzyl)-1H-indazol-3-yl]-N-(3-methoxypyridin-4-yl)-5-[1-(3,3,3-trifluoropropyl)piperidin-4-yl]pyrimidin-4-amine;
or an N-oxide, a salt, a tautomer or a stereoisomer of said compound, or a salt of said N-oxide, tautomer or stereoisomer.

8. (canceled)

9. A method for the treatment or prophylaxis of a disease, comprising administering to a patient in need thereof an effective amount of a compound of general formula (I) according to claim 1, or an N-oxide, a salt, a tautomer or a stereoisomer of said compound, or a salt of said N-oxide, tautomer or stereoisomer, wherein the disease is hyperproliferative disease and/or a disorder responsive to induction of cell death.

10. The method according to claim 9, wherein the hyperproliferative disease and/or disorder responsive to induction of cell death is a haematological tumour, solid tumour and/or metastases thereof.

11. The method according to according to claim 9, wherein the disease is a hyperproliferative disease, and wherein the hyperproliferative disease is cervical cancer.

12. A pharmaceutical composition comprising at least one compound of general formula (I) according to claim 1, or an N-oxide, a salt, a tautomer or a stereoisomer of said compound, or a salt of said N-oxide, tautomer or stereoisomer, together with at least one pharmaceutically acceptable carrier or auxiliary.

13. A method for the treatment of a haematological tumour, a solid tumour and/or metastases thereof, comprising administering to a patient in need thereof the composition according to claim 12.

14. A combination comprising one or more first active ingredients selected from a compound of general formula (I) according to claim 1, or an N-oxide, a salt, a tautomer or a stereoisomer of said compound, or a salt of said N-oxide, tautomer or stereoisomer, and one or more second active ingredients selected from chemotherapeutic anti-cancer agents and target-specific anti-cancer agents.

15. A method of preparing a compound of general formula (I), said method comprising the step of allowing an intermediate compound of general formula (1-16): 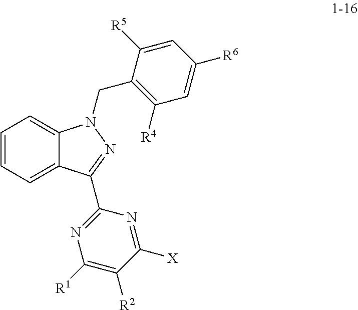
in which R¹, R², R⁴, R⁵, R⁶ are is as defined in claim 1, and X represents Cl or Br,
to react with a compound of the general formula (1-12), 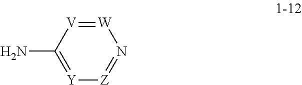
in which V, W, Y, and Z are is as defined in claim 1,
thereby giving a compound of general formula (I): 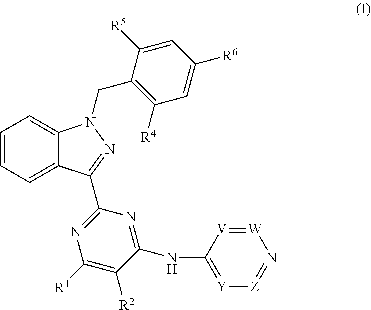
in which R¹, R², R⁴, R⁵, R⁶, V, W, Y, and Z are is as defined in claim 1.

16. A compound of formula (1-16): 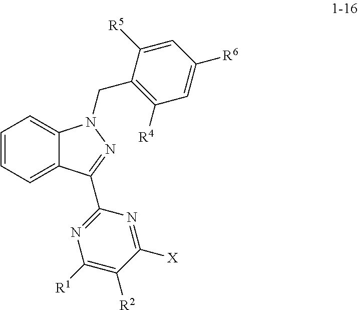
in which R¹, R², R⁴, R⁵, and R⁶ are is as defined in claim 1, and X represents Cl or Br.

17. (canceled)
